(19) United States
(12) Patent Application Publication
(10) Pub. No.: US 2025/0018049 A1
(43) Pub. Date: Jan. 16, 2025

(54) COMPOUNDS FOR DEGRADING CYCLIN-DEPENDENT KINASE 2 VIA UBIQUITIN PROTEOSOME PATHWAY

(71) Applicant: NIKANG THERAPEUTICS, INC., Wilmington, DE (US)

(72) Inventors: Jiping FU, Wilmington, DE (US); Yan LOU, Dallas, TX (US)

(21) Appl. No.: 18/711,875

(22) PCT Filed: Dec. 21, 2021
(86) PCT No.: PCT/US2021/064734

§ 371 Date: May 20, 2024

Related U.S. Application Data
(60) Provisional application No. 63/129,488, filed on Dec. 22, 2020.
(60) Provisional application No. 63/189,030, filed on May 14, 2021.

(51) Int. Cl. A61K 47/55; A61K 47/54

(52) U.S. Cl. CPC A61K 47/55; A61K 47/545

(57) ABSTRACT

The present disclosure provides certain bifunctional compounds that cause degradation of Cyclin-dependent kinase 2 (CDK2) via ubiquitin proteosome pathway and are therefore useful for the treatment of diseases mediated by CDK2. Also provided are pharmaceutical compositions containing such compounds and processes for preparing such compounds.

### FIELD OF THE DISCLOSURE

The present disclosure provides certain bifunctional compounds that cause degradation of Cyclin-dependent kinase 2 (CDK2) via ubiquitin proteosome pathway and are therefore useful for the treatment of diseases mediated by CDK2. Also provided are pharmaceutical compositions containing such compounds and processes for preparing such compounds.

### BACKGROUND

Cyclin-dependent kinases (CDKs) are cellular kinases that are critical for orchestrating signaling events such as DNA replication and protein synthesis to ensure faithful eukaryotic cell division and proliferation. To date, at least twenty-one mammalian CDKs have been identified (Malumbres M. Genome Biol. (2014) 15:122). Among these CDKs, at least CDK1/Cyclin B, CDK2/Cyclin E, CDK2/Cyclin A, CDK4/Cyclin D, CDK6/Cyclin D complexes are known to be important regulators of cell cycle progression; while other CDKs are important in regulating gene transcription, DNA repair, differentiation and apoptosis (see Morgan, D. O. Annu. Rev. Cell. Dev. Biol. (1997) 13: 261-291).

Due to their roles in regulating cell cycle and other essential cellular processes, increased activity or temporally abnormal activation of CDKs has been shown to result in the development of various types of cancer. Human tumor development is commonly associated with alterations in either the CDK proteins themselves or their regulators (Cordon-Cardo C. Am. J. Pathol. (1995) 147:545-560; Karp J E, Broder S. Nat. Med. (1995) 1:309-320; Hall M, Peters G. Adv. Cancer Res. (1996) 68:67-108). For example, amplifications of the regulatory subunits of CDKs and cyclins, and mutation, gene deletion, or transcriptional silencing of endogenous CDK inhibitory regulators have been reported (Smalley et al. Cancer Res. (2008) 68: 5743-52). A large body of research has established the role of these alterations in promoting tumorigenesis and progression. Thus, there has been great interest in the development of inhibitors of the Cyclin-dependent kinases (CDKs) for therapeutic purposes over the last two decades.

Selective CDK 4/6 inhibitors have changed the therapeutic management of hormone receptor-positive (HR+) metastatic breast cancer (MBC). Palbociclib, ribociclib, and abemaciclib, selective reversible inhibitors of CDK4 and CDK6, are approved for hormone receptor-positive (HR+) metastatic breast cancer in combination with endocrine therapies. Additional clinical trials with these CDK4/6 inhibitors are ongoing in both breast and other cancers, either as single agents or in combination with other therapeutics. (O'Leary et al. Nature Reviews (2016) 13:417-430). While CDK4/6 inhibitors have shown significant clinical efficacy in ER-positive metastatic breast cancer, the clinical benefit may be limited over time due to the development of primary or acquired resistance.

An important mechanism of resistance to CDK4/6 inhibitors is the abnormal activation of CDK2. It has been reported that high Cyclin E expression leads to overactivated CDK2/Cyclin E complex, which bypasses the requirement for CDK4/6 for cell cycle reentry (Asghar, U. et al. Clin. Cancer Res. (2017) 23:5561). In addition, it has been found that when CDK4/6 is inhibited, there is a noncanonical CDK2/cyclin D1 complex formation that promotes pRb phosphorylation recovery and drives cell cycle progression (Herrera-Abreu M T et al, Cancer Res. (2006) 15: 2301).

The CDK2/Cyclin E complex plays an important role in regulation of the G1/S transition, histone biosynthesis and centrosome duplication. Following the initial phosphorylation of Rb by Cdk4/6/cyclin D, Cdk2/Cyclin E further hyper-phosphorylates p-RB, releases E2F to transcribe genes required for S-phase entry. During S-phase, Cyclin E is degraded and CDK2 forms a complex with Cyclin A to promote phosphorylation of substrates that permit DNA replication and inactivation of E2F, for S-phase completion. (Asghar et al. Nat. Rev. Drug. Discov. (2015) 14: 130-146). In addition to cyclin bindings, the activity of CDK2 is also tightly regulated through its interaction with negative regulators, such as p21 and p27. In response to mitogenic stimulation, which signals optimal environment for cell cycle, p21 and p27 are phosphorylated and degraded, releasing the break on CDK2/Cyclin activation.

Cyclin E, the regulatory cyclin for CDK2, is frequently overexpressed in cancer, and its overexpression correlates with poor prognosis. For example, Cyclin E amplification or overexpression has been shown to associate with poor outcomes in breast cancer (Keyomarsi et al., N Engl J Med. (2002) 347:1566-75). Cyclin E2 (CCNE2) overexpression is associated with endocrine resistance in breast cancer cells and CDK2 inhibition has been reported to restore sensitivity to tamoxifen or CDK4/6 inhibitors in tamoxifen-resistant and CCNE2 overexpressing cells. (Caldon et al., Mol Cancer Ther. (2012)11:1488-99; Herrera-Abreu et al., Cancer Res. (2016)76:2301-2313). Cyclin E amplification also reportedly contributes to trastuzumab resistance in HER2+ breast cancer. (Scaltriti et al. Proc Natl Acad Sci. (2011) 108:3761-6). Cyclin E overexpression has also been reported to play a role in basal-like and triple negative breast cancer (TNBC), as well as inflammatory breast cancer (Elsawaf Z. et al. Breast Care (2011) 6:273-278; Alexander A. et al. Oncotarget (2017) 8:14897-14911.)

Amplification or overexpression of cyclin E1 (CCNE1) is also frequently found in ovarian, gastric, endometrial, uterus, bladder, esophagus, prostate, lung and other types of cancers (Nakayama et al. Cancer (2010) 116:2621-34; Etemadmoghadam et al. Clin Cancer Res (2013) 19: 5960-71; Au-Yeung et al. Clin. Cancer Res. (2017) 23:1862-1874; Ayhan et al. Modern Pathology (2017) 30: 297-303; Ooi et al. Hum Pathol. (2017) 61:58-67: Noske et al. Oncotarget (2017) 8: 14794-14805) and often correlates with poor clinical outcomes.

In some cancer types loss-of-function mutations in FBXW7, a component of SCF^{Fbw7} ubiquitin E3 ligase responsible for cyclin E degradation, also leads to cyclin E overexpression and CDK2 activation. Alternatively, certain cancer cells express a hyperactive, truncated form of cyclin E. In addition, cyclin A amplification and overexpression have also been reported in various cancers such as hepatocellular carcinomas, colorectal and breast cancers.

In contrast to the frequent upregulation of Cyclin E, the inhibitory regulators of CDK2, p21 and p27 are often abnormally downregulated in cancers. It is postulated that the loss or decrease of these key endogenous inhibitors leads to high and/or abnormal temporal activation of CDK2, thereby promoting oncogenic growth.

In addition, CDC25A and CDC25B, protein phosphatases responsible for the dephosphorylations that activate the CDK2, are overexpressed in various tumors. These various mechanisms of CDK2 activation have been validated using mouse cancer models. Furthermore, CDK2/cyclin E phosphorylates oncogenic Myc to oppose ras-induced senescence, highlighting the importance of CDK2 in myc/ras-induced tumorigenesis. Inactivation of CDK2 has been shown to be synthetically lethal to myc over-expressing cancer cells.

Recently, pharmacologic inhibition or genetic deletion of CDK2 was shown to preserve hearing function in animal models treated with cisplatin or noise (Teitz T et al. J Exp Med. 2018 Apr. 2; 215(4):1187-1203). Mechanistically, inhibition of CDK2 kinase activity reduces cisplatin-induced mitochondrial production of reactive oxygen species, thereby enhancing survival of inner ear cells. Therefore, in addition to anti-tumor therapies, CDK2 inhibition can also be used as a promising preventive treatment for noise-, cisplatin-, or antibiotic-induced or age-related hearing loss, for which no Food and Drug Administration-approved drugs are currently available.

Currently, there are a few CDK2 inhibitors in early phase of clinical trials. For example, Dinaciclib (MK-7965) which inhibits CDK1, CDK2, CDK5 and CDK9 is in clinical development for solid tumors and hematological cancers in combination with other agents; CYC065, which potently inhibits CDK2, CDK3, CDK4, CDK9 and moderately inhibits CDK1. CDK5 and CDK7, is being investigated for the treatment of refractory CLL and other cancers; and PF-06873600, a CDK2 inhibitor with activities against other CDKs, is in clinical trial for the treatment of breast cancer either as single agent or in combination with endocrine therapies.

As an alternative to inhibition, removal of CDK2 protein would eliminate CDK2 activity as well as any protein interaction or scaffolding function of CDK2. Accordingly, there is a need for bifunctional molecules that could recruit CDK2 to a ubiquitin ligase and thereby causing ubiquitylation and proteasomal degradation of CDK2. The present disclosure fulfills this and related needs.

### SUMMARY

In a first aspect, provided is a compound of Formula (IA′):

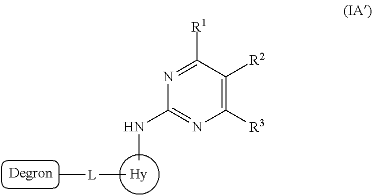

wherein:
Degron is an E3 ligase ligand of formula (i) or (ii);

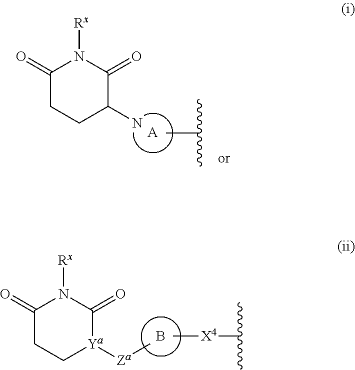

where:
   R^{x} is hydrogen, alkyl, cycloalkyl, or alkylcarbonyloxy;
   Y^{a} is CH or N;
   Z^{a} is a bond, —CH₂—, —NH—, O, or —NHC(O)— where NH of —NHC(O)— is attached to Y^{a};
   ring A is a group of formula (a), (b), or (c):

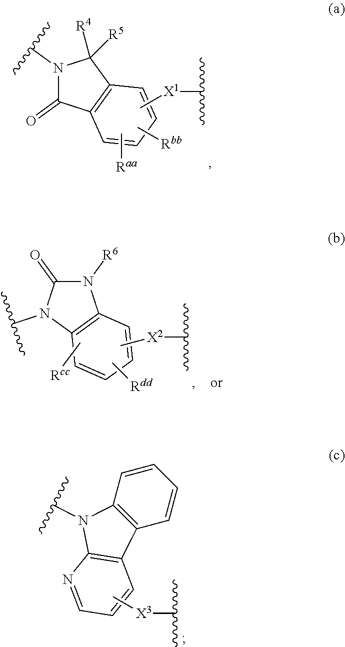

where:
   R^{aa}, R^{bb}, R^{cc}, and R^{dd} are independently selected from hydrogen, alkyl, alkoxy, halo, haloalkyl, haloalkoxy, and cyano;
   R⁴ and R³ are independently hydrogen or alkyl; or R⁴ and R⁵ together with the carbon to which they are attached form >C═O; and
   R⁶ is hydrogen or alkyl;
ring B is phenylene, cyclylaminylene, a 5- or 6-membered monocyclic heteroarylene, or a 9- or 10-membered fused bicyclic heteroarylene, wherein each heteroarylene ring contains one to three nitrogen ring atoms and further wherein the phenylene, cyclylaminylene, and heteroarylene rings are independently substituted with R^{ee} and R^{ff} independently selected from hydrogen, alkyl, alkoxy, halo, haloalkyl, haloalkoxy, and cyano; and
   X¹, X², X³ and X⁴ are independently a bond, -alkylene-, —O—, —(O-alkylene)-, -(alkylene-O)—, —(NR^{s}-alkylene)-, -(alkylene-NR^{t})—, —C≡C—, —NH—, —N(alkyl)-, —C(═O)—, —NR^{u}C(═O)—, or —C(═O)NR^{v}— where R^{s}, R^{t}, R^{u}, and R^{v} are independently hydrogen, alkyl, or cycloalkyl and each alkylene is optionally substituted with one or two fluoro; and
Hy is cycloalkylene, arylene, heterocyclylene, bicyclic heterocyclylene, spiro heterocyclylene, bridged heterocyclylene, or fused heterocyclylene, where each of the aforementioned rings is optionally substituted with one, two, or three substituents independently selected from deuterium, alkyl, halo, haloalkyl, alkoxy, and hydroxy;
R¹ or R³ is hydrogen, provided that:
(i) when R¹ is hydrogen; then
   R² and R³ together with the carbon atoms to which they are attached form a ring of formula (d1):

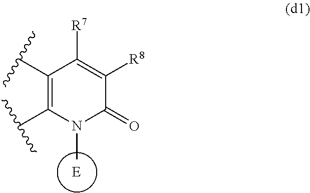

where:
R⁷ is hydrogen, alkyl, or haloalkyl;
R⁸ is hydrogen, cyano, halo, NH₂, alkyl, or haloalkyl where alkyl and haloalkyl are optionally substituted with R^{a} and R^{b} independently selected from hydroxy, cyano, alkoxy, haloalkoxy, C(O)NH₂, and —C(O)OH; and
ring E is bicyclic cycloalkyl, bridged cycloalkyl, or a ring of formula:

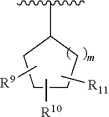

where m is 1, 2, or 3 and R⁹, R¹⁰, and R¹¹ are independently selected from hydrogen, deuterium, alkyl, haloalkyl, alkoxy, haloalkoxy, cyano, halo, and hydroxy; or when R⁹ and R¹⁰ are attached to the same carbon, R⁹ and R¹⁰ together with the carbon atom to which they are attached can form cycloalkylene or heterocyclylene; and
(ii) when R³ is hydrogen, then:
(A): R¹ is a ring of formula (e):

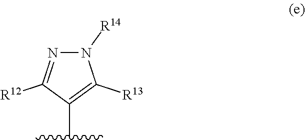

where:
R¹² is hydrogen, deuterium, alkyl, haloalkyl, cycloalkyl, halo, haloalkoxy, or cyano and R² is alkyl, halo, haloalkyl, cycloalkyl, or cyano; or
R¹² is alkyl, halo, haloalkyl, cycloalkyl, or cyano and R² is hydrogen, deuterium, alkyl, haloalkyl, cycloalkyl, halo, haloalkoxy, or cyano;
R¹³ is hydrogen, deuterium, alkyl, alkenyl, alkynyl, cycloalkyl, cycloalkylalkyl, halo, haloalkyl, cyano, aralkyl, heteroaralkyl, heterocyclyl, fused heterocyclyl, or heterocyclylalkyl wherein the alkyl, haloalkyl, cycloalkyl, heterocyclyl, and the ring portion of cycloalkylalkyl, aralkyl, heteroaralkyl, and heterocyclylalkyl are substituted with R^{d}, R^{e}, and R^{f} independently selected from hydrogen, deuterium, alkyl, halo, haloalkyl, cycloalkyl, cyano, hydroxy, alkoxy, acyl, aminocarbonyl, aminosulfonyl, carboxy, amino, optionally substituted aryl, optionally substituted aralkyl, optionally substituted heteroaryl, optionally substituted heteroaralkyl, optionally substituted heterocyclyl, and optionally substituted heterocyclylalkyl; and
R¹⁴ is cycloalkyl, cycloalkylalkyl, haloalkyl, hydroxyalkyl, aminocarbonylalkyl, aryl, aralkyl, heteroaryl, heteroaralkyl, heterocyclyl, or heterocyclylalkyl wherein cycloalkyl, aryl, heteroaryl, heterocyclyl, and the ring portion of cycloalkylalkyl, aralkyl, heteroaralkyl, and heterocyclylalkyl are substituted with R^{g}, R^{h}, and Rⁱ independently selected from hydrogen, alkyl, halo, haloalkyl, cycloalkyl, cycloalkylalkyl, aminoalkyl, (amino)deuteroalkyl, cyano, hydroxy, alkoxy, acyl, aminocarbonyl, aminosulfonyl, carboxy, amino, optionally substituted aryl, optionally substituted aralkyl, optionally substituted heteroaryl, optionally substituted heteroaralkyl, optionally substituted heterocyclyl, and optionally substituted heterocyclylalkyl; or
(B): R¹ and R² together with the carbons to which they are attached form a ring of formula (f):

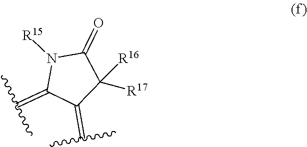

wherein:
R¹⁵ is alkyl, alkenyl, alkynyl, cycloalkyl, cycloalkylalkyl, haloalkyl, aryl, aralkyl, heteroaryl, heteroaralkyl, heterocyclyl, fused heterocyclyl, or heterocyclylalkyl, where each of the aforementioned groups is substituted with R^{j}, R^{k}, and R^{l} independently selected from hydrogen, deuterium, alkyl, halo, haloalkyl, cycloalkyl, cyano, hydroxy, alkoxy, acyl, aminocarbonyl, aminosulfonyl, carboxy, amino, optionally substituted aryl, optionally substituted aralkyl, optionally substituted heteroaryl, optionally substituted heteroaralkyl, optionally substituted heterocyclyl, and optionally substituted heterocyclylalkyl;
R¹⁶ and R¹⁷ are independently alkyl, cycloalkyl, haloalkyl, phenyl, heteroaryl, heteroaralkyl, heterocyclyl, or heterocyclylalkyl, where each of the aforementioned groups is substituted with R^{m}, Rⁿ, and R^{o} independently selected from hydrogen, deuterium, alkyl, halo, haloalkyl, cycloalkyl, cyano, hydroxy, alkoxy, acyl, aminocarbonyl, aminosulfonyl, carboxy, amino, optionally substituted aryl, optionally substituted aralkyl, optionally substituted heteroaryl, optionally substituted heteroaralkyl, optionally substituted heterocyclyl, and optionally substituted heterocyclylalkyl; or
R¹⁶ and R¹⁷ together with the carbon atom to which they are attached form cycloalkylene or heterocyclylene, where each of the aforementioned rings is substituted with R^{p}, R^{q}, and R^{r} independently selected from hydrogen, deuterium, alkyl, cycloalkyl, halo, haloalkyl, hydroxy, alkoxy, haloalkoxy, amino, alkylamino, dialkylamino, alkylsulfonyl, carboxy, alkylcarbonyl, alkoxycarbonyl, cyano, cyanoalkyl, hydroxyalkyl, and alkoxyalkyl; and
L is —Z¹-Z²—Z³—Z⁴-Z⁵—Z⁶— where:
Z¹ is a bond, alkylene, —C(O)NR—, —NR′(CO)—, —S(O)₂NR—, —NR′S(O)₂—, —(O-alkylene)ₐ-, -(alkylene-O)ₐ—, phenylene, monocyclic heteroarylene, or heterocyclylene, where each ring is optionally substituted with one or two substituents independently selected from alkyl, alkoxy, halo, haloalkyl, and haloalkoxy;
Z² is a bond, alkylene, alkynylene, —C(O)—, —C(O)N(R)—, —NR′(CO)—, —(O-alkylene)_{b}-, -(alkylene-O)_{b}—, —O(CH₂)₇—, —O(CH₂)₃—, cycloalkylene, -heterocyclylene, where each ring is optionally substituted with one or two substituents independently selected from alkyl, alkoxy, halo, haloalkyl, and haloalkoxy;
Z³ is a bond, alkylene, alkynylene, —C(O)NR—, —NR′(CO)—, —O—, —NR″—, —(O-alkylene)_{c}-, -(alkylene-O)_{c}—, cycloalkylene, spiro cyclolalkylene, phenylene, monocyclic heteroarylene, heterocyclylene, bicyclic heterocyclylene, bridged heterocyclylene, fused heterocyclylene, spiro heterocyclylene, or 11 to 13 membered spiro heterocyclylene, where each ring is optionally substituted with one or two substituents independently selected from alkyl, alkoxy, halo, haloalkyl, and haloalkoxy;
Z⁴ is a bond, alkylene, alkynylene, -(alkylene-NR″)—, —O—, —C(O)—, —NR″—, —(O-alkylene)ₐ-, -(alkylene-O)_{d}—, cycloalkylene, spiro cyclolalkylene, phenylene, heteroarylene, heterocyclylene, fused heterocyclylene, bridged heterocyclylene, or spiro heterocyclylene, where each ring is optionally substituted with one or two substituents independently selected from alkyl, alkoxy, halo, haloalkyl, and haloalkoxy;
Z⁵ is a bond, -alkylene, —NR″—, —O—, —C(O)—, —S(O)₂—, —NR′(CO)—, —C(O)NR—, phenylene, monocyclic heteroarylene, or heterocycylene, where each ring is optionally substituted with one or two substituents independently selected from alkyl, alkoxy, halo, haloalkyl, and haloalkoxy, and
Z⁶ is a bond, alkylene, —NR″—, —O—, -(alkylene-O)—, —C(O)—, —S(O)₂—, —NR′(CO)—, or —C(O)NR—;
where each R, R′ and R″ is independently hydrogen or alkyl, each a, b, c, and d is independently an integer selected from 1 to 6 inclusive, and each alkylene is optionally substituted with one to four substituents where one, two, or three substituents are independently selected from fluoro and deuterium, and the fourth substituent is carboxy; provided that at least one of —Z¹-Z²—Z³—Z⁴-Z⁵—Z⁶— is not a bond; or
a pharmaceutically acceptable salt thereof.

In a second aspect, provided is a compound of Formula (IA):

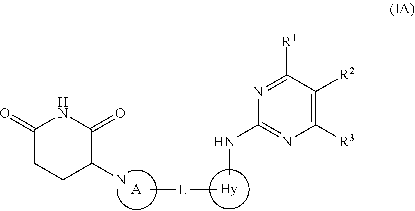

wherein:
ring A is a group of formula (a), (b), or (c):

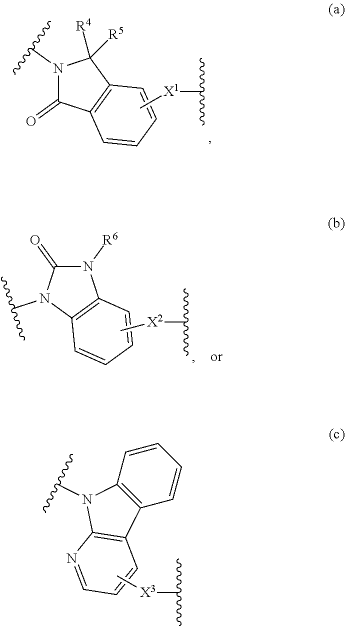

where:
R⁴ and R⁵ are independently hydrogen or alkyl; or R⁴ and R⁵ together with the carbon to which they are attached form >C═O;
R⁶ is hydrogen or alkyl; and
X¹, X², and X³ are independently a bond, -alkylene-, —O—, —(O-alkylene)-, -(alkylene-O)—, —(NR^{s}-alkylene)-, -(alkylene-NR^{t})—, —C≡C—, —NH—, —N(alkyl)-, —C(═O)—, —NR^{u}C(═O)—, or —C(═O)NR^{v}— where R^{s}, R^{t}, R^{u}, and R^{v} are independently hydrogen, alkyl, or cycloalkyl and each alkylene is optionally substituted with one or two fluoro;
Hy is cycloalkylene, arylene, heterocyclylene, bicyclic heterocyclylene, spiro heterocyclylene, bridged heterocyclylene, or fused heterocyclylene, where each of the aforementioned ring is optionally substituted with one or two substituents independently selected from alkyl, halo, haloalkyl, alkoxy, and hydroxy;
R¹ or R³ is hydrogen, provided that:
(i) when R¹ is hydrogen; then
   R² and R³ together with the carbon atoms to which they are attached form a ring of formula (d):

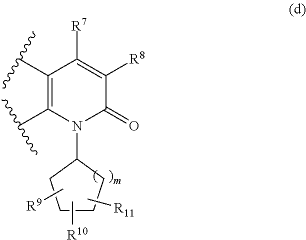

where:
m is 1, 2, or 3;
R⁷ is hydrogen, alkyl, or haloalkyl;
R⁸ is hydrogen, cyano, halo, NH₂, difluoromethyl, alkyl, or haloalkyl where alkyl and haloalkyl are substituted with Ra and R^{b} independently selected from hydroxy, cyano, alkoxy, haloalkoxy, C(O)NH₂, and —C(O)OH; and
R⁹, R¹⁰, and R¹¹ are independently selected from hydrogen, alkyl, haloalkyl, alkoxy, haloalkoxy, cyano, halo, and hydroxy; or
when R⁹ and R¹⁰ are attached to the same carbon, R⁹ and R¹⁰ together with the carbon atom to which they are attached can form cycloalkylene or heterocyclylene; and
(ii) when R³ is hydrogen, then:
(A): R¹ is a ring of formula (e):

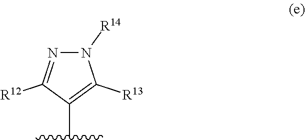

where:
R¹² is hydrogen, deuterium, alkyl, haloalkyl, cycloalkyl, halo, haloalkoxy, or cyano and R² is alkyl, halo, haloalkyl, cycloalkyl, or cyano; or
R¹² is alkyl, halo, haloalkyl, cycloalkyl, or cyano and R² is hydrogen, deuterium, alkyl, haloalkyl, cycloalkyl, halo, haloalkoxy, or cyano;
R¹³ is hydrogen, deuterium, alkyl, alkenyl, alkynyl, cycloalkyl, cycloalkylalkyl, halo, haloalkyl, cyano, aralkyl, heteroaralkyl, heterocyclyl, fused heterocyclyl, or heterocyclylalkyl wherein the alkyl, haloalkyl, cycloalkyl, heterocyclyl, and the ring portion of cycloalkylalkyl, aralkyl, heteroaralkyl, and heterocyclylalkyl are substituted with R^{d}, R^{e}, and R^{f} independently selected from hydrogen, deuterium, alkyl, halo, haloalkyl, cycloalkyl, cyano, hydroxy, alkoxy, acyl, aminocarbonyl, aminosulfonyl, carboxy, amino, optionally substituted aryl, optionally substituted aralkyl, optionally substituted heteroaryl, optionally substituted heteroaralkyl, optionally substituted heterocyclyl, and optionally substituted heterocyclylalkyl; and
R¹⁴ is cycloalkyl, cycloalkylalkyl, haloalkyl, hydroxyalkyl, aminocarbonylalkyl, aryl, aralkyl, heteroaryl, heteroaralkyl, heterocyclyl, or heterocyclylalkyl wherein cycloalkyl, aryl, heteroaryl, heterocyclyl, and the ring portion of cycloalkylalkyl, aralkyl, heteroaralkyl, and heterocyclylalkyl are substituted with R^{g}, R^{h}, and Rⁱ independently selected from hydrogen, alkyl, halo, haloalkyl, cycloalkyl, cycloalkylalkyl, aminoalkyl, (amino)deuteroalkyl, cyano, hydroxy, alkoxy, acyl, aminocarbonyl, aminosulfonyl, carboxy, amino, optionally substituted aryl, optionally substituted aralkyl, optionally substituted heteroaryl, optionally substituted heteroaralkyl, optionally substituted heterocyclyl, and optionally substituted heterocyclylalkyl; or
(B): R¹ and R² together with the carbons to which they are attached form a ring of formula (f):

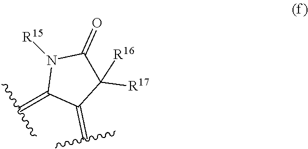

wherein:
R¹⁵ is alkyl, alkenyl, alkynyl, cycloalkyl, cycloalkylalkyl, haloalkyl, aryl, aralkyl, heteroaryl, heteroaralkyl, heterocyclyl, fused heterocyclyl, or heterocyclylalkyl, where each of the aforementioned groups is substituted with R^{j}, R^{k}, and R^{l} independently selected from hydrogen, deuterium, alkyl, halo, haloalkyl, cycloalkyl, cyano, hydroxy, alkoxy, acyl, aminocarbonyl, aminosulfonyl, carboxy, amino, optionally substituted aryl, optionally substituted aralkyl, optionally substituted heteroaryl, optionally substituted heteroaralkyl, optionally substituted heterocyclyl, and optionally substituted heterocyclylalkyl;
R¹⁶ and R¹⁷ are independently alkyl, cycloalkyl, haloalkyl, phenyl, heteroaryl, heteroaralkyl, heterocyclyl, or heterocyclylalkyl, where each of the aforementioned groups is substituted with R^{m}, Rⁿ, and R^{o} independently selected from hydrogen, deuterium, alkyl, halo, haloalkyl, cycloalkyl, cyano, hydroxy, alkoxy, acyl, aminocarbonyl, aminosulfonyl, carboxy, amino, optionally substituted aryl, optionally substituted aralkyl, optionally substituted heteroaryl, optionally substituted heteroaralkyl, optionally substituted heterocyclyl, and optionally substituted heterocyclylalkyl; or
R¹⁶ and R¹⁷ together with the carbon atom to which they are attached form cycloalkylene or heterocylylene, where each of the aforementioned ring is substituted with R^{p}, R^{q}, and R^{r} independently selected from hydrogen, deuterium, alkyl, cycloalkyl, halo, haloalkyl, hydroxy, alkoxy, haloalkoxy, amino, alkylamino, dialkylamino, alkylsulfonyl, carboxy, alkylcarbonyl, alkoxycarbonyl, cyano, cyanoalkyl, hydroxyalkyl, and alkoxyalkyl; and
L is —Z¹-Z²—Z³—Z⁴-Z⁵—Z⁶— where:
Z¹ is a bond, alkylene, —C(O)NR—, —NR′(CO)—, —S(O)₂NR—, —NR′S(O)₂—, —(O-alkylene)ₐ-, -(alkylene-O)ₐ—, phenylene, monocyclic heteroarylene, or heterocyclylene, where each ring is optionally substituted with one or two alkyl;
Z² is a bond, alkylene, alkynylene, —C(O)—, —C(O)N(R)—, —NR′(CO)—, —(O-alkylene)_{b}-, -(alkylene-O)_{b}—, —O(CH₂)₇—, —O(CH₂)₃—, cycloalkylene, or heterocyclylene, where each ring is optionally substituted with one or two alkyl;
Z³ is a bond, alkylene, alkynylene, —C(O)NR—, —NR′(CO)—, —O—, —NR″—, —(O-alkylene)_{c}-, -(alkylene-O)ₑ—, cycloalkylene, spiro cyclolalkylene, phenylene, monocyclic heteroarylene, heterocyclylene, bicyclic heterocyclylene, bridged heterocyclylene, fused heterocyclylene, spiro heterocyclylene, or 11 to 13 membered spiro heterocyclylene, where each ring is optionally substituted with one or two alkyl;
Z⁴ is a bond, alkylene, alkynylene, -(alkylene-NR″)—, —O—, —C(O)—, —NR″—, —(O-alkylene)ₐ-, -(alkylene-O)_{d}—, cycloalkylene, spiro cyclolalkylene, phenylene, heteroarylene, heterocyclylene, fused heterocyclylene, or spiro heterocyclylene, where each ring is optionally substituted with one or two alkyl;
Z⁵ is a bond, -alkylene, —NR″—, —O—, —C(O)—, —S(O)₂—, —NR′(CO)—, —C(O)NR—, phenylene, monocyclic heteroarylene, or heterocycylene, where each ring is optionally substituted with alkyl, and
Z⁶ is a bond, alkylene, —NR″—, —O—, -(alkylene-O)—, —C(O)—, —S(O)₂—, —NR′(CO)—, or —C(O)NR—;
where each R, R′ and R″ is independently hydrogen or alkyl, each a, b, c, and d is independently an integer selected from 1 to 6 inclusive, and each alkylene is optionally substituted with one, two, or three fluoro or a carboxy; provided that at least one of —Z¹-Z²—Z³—Z⁴-Z⁵—Z⁶— is not a bond; or
a pharmaceutically acceptable salt thereof.

In a third aspect, provided is a compound of Formula (I):

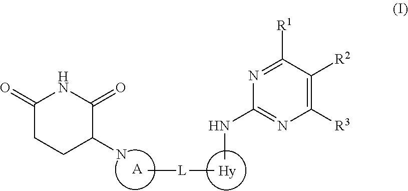

wherein:
ring A is a group of formula (a), (b), or (c):

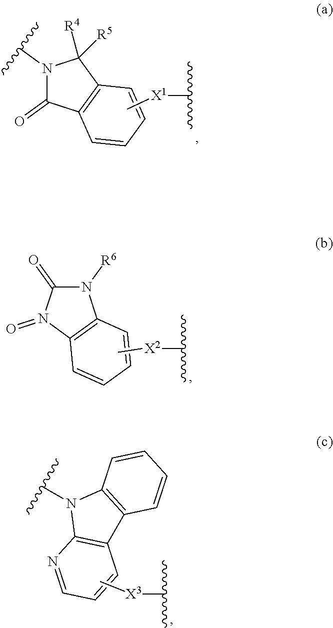

where:
R⁴ and R³ are independently hydrogen or alkyl; or R⁴ and R³ together with the carbon to which they are attached form >C═O;
R⁶ is hydrogen or alkyl; and
X¹, X², and X³ are independently a bond, -alkylene-, —O—, —(O-alkylene)-, -(alkylene-O)—, —(NR′-alkylene)-, -(alkylene-NR^{t})—, —C≡C—, —NH—, —N(alkyl)-, —C(═O)—, —NR^{u}C(═O)—, or —C(═O)NR^{v}— where R^{s}, R^{t}, R^{u}, and R^{v} are independently hydrogen or alkyl and each alkylene is optionally substituted with one or two fluoro;
Hy is cycloalkylene, arylene, heterocyclylene, bicyclic heterocyclylene, spiro heterocyclylene, bridged heterocyclylene, or fused heterocyclylene, where each of the aforementioned ring is optionally substituted with one or two substituents independently selected from alkyl, halo, haloalkyl, alkoxy, and hydroxy;
R¹ or R³ is hydrogen, provided that:
(i) when R¹ is hydrogen; then
   R² and R³ together with the carbon atoms to which they are attached form a ring of formula (d):

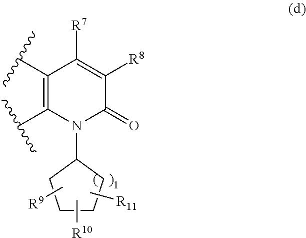

where:
m is 1, 2, or 3;
R⁷ is hydrogen, alkyl, or haloalkyl;
R⁸ is hydrogen, halo, NH₂, difluoromethyl, alkyl, or haloalkyl where alkyl and haloalkyl are substituted with Ra and R^{b} independently selected from hydroxy, cyano, alkoxy, haloalkoxy, C(O)NH₂, and

### —C(O)OH; and

R⁹, R¹⁰, and R¹¹ are independently selected from hydrogen, alkyl, haloalkyl, alkoxy, haloalkoxy, cyano, halo, and hydroxy; or
when R⁹ and R¹⁰ are attached to the same carbon, R⁹ and R¹⁰ together with the carbon atom to which they are attached can form cycloalkylene or heterocyclylene; and
(ii) when R³ is hydrogen, then:
(A): R¹ is a ring of formula (e):

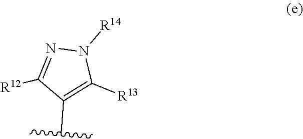

where:
R¹² is hydrogen, deuterium, alkyl, haloalkyl, cycloalkyl, halo, haloalkoxy, or cyano and R² is alkyl, halo, haloalkyl, cycloalkyl, or cyano; or
R¹² is alkyl, halo, haloalkyl, cycloalkyl, or cyano and R² is hydrogen, deuterium, alkyl, haloalkyl, cycloalkyl, halo, haloalkoxy, or cyano;
R¹³ is hydrogen, deuterium, alkyl, alkenyl, alkynyl, cycloalkyl, cycloalkylalkyl, halo, haloalkyl, cyano, aralkyl, heteroaralkyl, heterocyclyl, fused heterocyclyl, or heterocyclylalkyl wherein the alkyl, haloalkyl, cycloalkyl, heterocyclyl, and the ring portion of cycloalkylalkyl, aralkyl, heteroaralkyl, and heterocyclylalkyl are substituted with R^{d}, R^{e}, and R^{f} independently selected from hydrogen, deuterium, alkyl, halo, haloalkyl, cycloalkyl, cyano, hydroxy, alkoxy, acyl, aminocarbonyl, aminosulfonyl, carboxy, amino, optionally substituted aryl, optionally substituted aralkyl, optionally substituted heteroaryl, optionally substituted heteroaralkyl, optionally substituted heterocyclyl, and optionally substituted heterocyclylalkyl; and
R¹⁴ is cycloalkyl, cycloalkylalkyl, haloalkyl, hydroxyalkyl, aminocarbonylalkyl, aryl, aralkyl, heteroaryl, heteroaralkyl, heterocyclyl, or heterocyclylalkyl wherein cycloalkyl, aryl, heteroaryl, heterocyclyl, and the ring portion of cycloalkylalkyl, aralkyl, heteroaralkyl, and heterocyclylalkyl are substituted with R^{g}, R^{h}, and Rⁱ independently selected from hydrogen, alkyl, halo, haloalkyl, cycloalkyl, cycloalkylalkyl, aminoalkyl, (amino)deuteroalkyl, cyano, hydroxy, alkoxy, acyl, aminocarbonyl, aminosulfonyl, carboxy, amino, optionally substituted aryl, optionally substituted aralkyl, optionally substituted heteroaryl, optionally substituted heteroaralkyl, optionally substituted heterocyclyl, and optionally substituted heterocyclylalkyl; or
(B): R¹ and R² together with the carbons to which they are attached form a ring of formula (f):

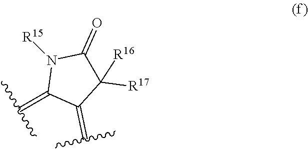

wherein:
R¹⁵ is alkyl, alkenyl, alkynyl, cycloalkyl, cycloalkylalkyl, haloalkyl, aryl, aralkyl, heteroaryl, heteroaralkyl, heterocyclyl, fused heterocyclyl, or heterocyclylalkyl, where each of the aforementioned groups is substituted with R^{j}, R^{k}, and R^{l} independently selected from hydrogen, deuterium, alkyl, halo, haloalkyl, cycloalkyl, cyano, hydroxy, alkoxy, acyl, aminocarbonyl, aminosulfonyl, carboxy, amino, optionally substituted aryl, optionally substituted aralkyl, optionally substituted heteroaryl, optionally substituted heteroaralkyl, optionally substituted heterocyclyl, and optionally substituted heterocyclylalkyl;
R¹⁶ and R¹⁷ are independently alkyl, cycloalkyl, haloalkyl, phenyl, heteroaryl, heteroaralkyl, heterocyclyl, or heterocyclylalkyl, where each of the aforementioned groups is substituted with R^{m}, Rⁿ, and R^{o} independently selected from hydrogen, deuterium, alkyl, halo, haloalkyl, cycloalkyl, cyano, hydroxy, alkoxy, acyl, aminocarbonyl, aminosulfonyl, carboxy, amino, optionally substituted aryl, optionally substituted aralkyl, optionally substituted heteroaryl, optionally substituted heteroaralkyl, optionally substituted heterocyclyl, and optionally substituted heterocyclylalkyl; or
R¹⁶ and R¹⁷ together with the carbon atom to which they are attached form cycloalkylene or heterocylylene, where each of the aforementioned ring is substituted with R^{p}, R^{q}, and R^{r} independently selected from hydrogen, deuterium, alkyl, cycloalkyl, halo, haloalkyl, hydroxy, alkoxy, haloalkoxy, amino, alkylamino, dialkylamino, alkylsulfonyl, carboxy, alkylcarbonyl, alkoxycarbonyl, cyano, cyanoalkyl, hydroxyalkyl, and alkoxyalkyl; and
L is —Z¹-Z²—Z³—Z⁴-Z⁵—Z⁶— where:
Z¹ is a bond, alkylene, —C(O)NR—, —NR′(CO)—, —(O-alkylene)ₐ-, -(alkylene-O)ₐ—, phenylene, or heterocyclylene, where each ring is optionally substituted with one or two alkyl;
Z² is a bond, alkylene, —C(O)—, —C(O)N(R)—, —NR′(CO)—, —(O-alkylene)_{b}-, -(alkylene-O)_{b}—, cycloalkylene, or heterocyclylene, where each ring is optionally substituted with one or two alkyl;
Z³ is a bond, alkylene, alkynylene, —C(O)NR—, —NR′(CO)—, —O—, —NR″—, —(O-alkylene)_{c}-, -(alkylene-O)ₑ—, cycloalkylene, phenylene, monocyclic heteroarylene, heterocyclylene, fused heterocyclylene, or spiro heterocyclylene, where each ring is optionally substituted with one or two alkyl;
Z⁴ is a bond, alkylene, alkynylene, -(alkylene-NR″)—, —O—, —C(O)—, —NR″—, —(O-alkylene)ₐ-, -(alkylene-O)_{d}—, cycloalkylene, phenylene, monocyclic heteroarylene, heterocyclylene, fused heterocyclylene, or spiro heterocyclylene, where each ring is optionally substituted with one or two alkyl;
Z⁵ is a bond, -alkylene, —NR″—, —O—, —C(O)—, —S(O)₂—, —NR′(CO)—, —C(O)NR—, phenylene, monocyclic heteroarylene, or heterocycylene, where each ring is optionally substituted with alkyl, and
Z⁶ is a bond, alkylene, —NR″—, —O—, -(alkylene-O)—, —C(O)—, —S(O)₂—, —NR′(CO)—, or —C(O)NR—;
where each R, R′ and R″ is independently hydrogen or alkyl, each a, b, c, and d is independently an integer selected from 1 to 6 inclusive, and each alkylene is optionally substituted with one or two fluoro; provided that at least one of —Z¹-Z²—Z³—Z⁴-Z⁵—Z⁶— is not a bond; or
   a pharmaceutically acceptable salt thereof.

Compounds of Formula (I) are a subset of compounds of Formula (IA′) and (IA) and compounds of Formula (IA) are a subset of compounds of Formula (IA′).

In a fourth aspect, provided is a method of treating a disease mediated by CDK2 in a patient, preferably the patient is in need of such treatment, which method comprises administering to the patient, preferably a patient in need of such treatment, a therapeutically effective amount of a compound of Formula (IA′), (IA), or (I) (or any of the embodiments thereof described herein) or a pharmaceutically acceptable salt thereof. In a first embodiment of the fourth aspect, the disease is cancer. In a second subembodiment of the fourth aspect the disease is cancer selected from lung cancer (e.g., adenocarcinoma, small cell lung cancer and non-small cell lung carcinomas, parvicellular and non-parvicellular carcinoma, bronchial carcinoma, bronchial adenoma, pleuropulmonary blastoma), skin cancer (e.g., melanoma, squamous cell carcinoma, Kaposi sarcoma, Merkel cell skin cancer), bladder cancer, breast cancer, cervical cancer, colorectal cancer, cancer of the small intestine, colon cancer, rectal cancer, cancer of the anus, endometrial cancer, gastric cancer, head and neck cancer (e.g., cancers of the larynx, hypopharynx, nasopharynx, oropharynx, lips, and mouth), liver cancer (e.g., hepatocellular carcinoma, cholangiocellular carcinoma), ovarian cancer, prostate cancer, testicular cancer, uterine cancer, esophageal cancer, gall bladder cancer, pancreatic cancer (e.g., exocrine pancreatic carcinoma), stomach cancer, thyroid cancer, and parathyroid cancer. In a third embodiment of the fourth aspect, the cancers are those that are resistant to CDK4/6 inhibitors through CDK2-mediated mechanisms. In a third embodiment of the fourth aspect, the therapeutically effective amount of a compound of Formula (IA′), (IA), or (I), or a pharmaceutically acceptable salt thereof, is administered in a pharmaceutical composition.

In a fifth aspect, provided is a method of treating noise-, cisplatin-, antibiotic-induced- or age-related hearing loss, which method comprises administering to the patient, preferably a patient in need of such treatment, a therapeutically effective amount of a compound of Formula (IA′), (IA), or (I) (or any of the embodiments thereof described herein) or a pharmaceutically acceptable salt thereof. In some embodiments, the amount of hearing loss is reduced when compared to an age-matched control. In some embodiments, the hearing loss is prevented when compared to an age-matched control.

In a sixth aspect, provided is a pharmaceutical composition comprising a compound of Formula (IA′), (IA), or (I) (or any of the embodiments thereof described herein) or a pharmaceutically acceptable salt thereof; and a pharmaceutically acceptable excipient.

In a seventh aspect, provided is a compound of Formula (IA′), (IA), (I), (or any embodiments thereof described herein) or a pharmaceutically acceptable salt thereof for use as a medicament. In one embodiment, the compound Formula (IA′), (IA), or (I) (and any embodiments thereof described herein) or a pharmaceutically acceptable salt thereof is useful for the treatment of one or more of diseases disclosed in the fourth aspect above.

In a eighth aspect, provided is the use of a compound of Formula (IA′), (IA), or (I) or a pharmaceutically acceptable salt thereof (and any embodiments thereof disclosed herein) in the manufacture of a medicament for treating a disease in a patient in which the activity of CDK2 contributes to the pathology and/or symptoms of the disease. In one embodiment the disease is one or more of diseases disclosed in the fourth aspect above.

In an ninth aspect, provided is a method of degrading CDK2 via ubiquitin proteosome pathway which method comprises contacting CDK2 with a compound of Formula (IA′), (IA), or (I) (or any of the embodiments thereof described herein) or a pharmaceutically acceptable salt thereof; or contacting CDK2 with a pharmaceutical composition comprising a compound of Formula (IA′), (IA), or (I) (or any of the embodiments thereof described herein) or a pharmaceutically acceptable salt thereof, and a pharmaceutically acceptable excipient.

In the aforementioned aspect involving the treatment of cancer, further embodiments are provided comprising administering the compound of Formula (IA′), (I), or (IA) or a pharmaceutically acceptable salt thereof (or any embodiments thereof disclosed herein) in combination with at least one additional anticancer agent. When combination therapy is used, the agents can be administered simultaneously or sequentially.

In a tenth aspect, provided is a compound of Formula (II):

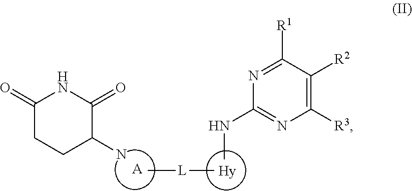

wherein the

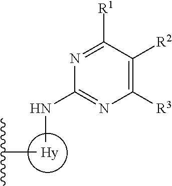

portion of the compound of Formula (II) is (i)

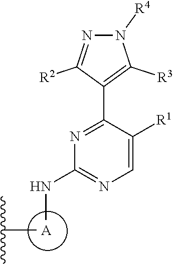

where ring A, R¹, R², R³ and R⁴ are as defined in PCT application publication No. 2020/180959 (and any embodiment thereof disclosed therein) or (ii)

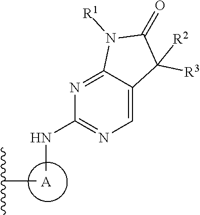

where ring A, R¹, R², and R³ are as defined in PCT application publication No. 2020/168197 (and any embodiment thereof disclosed therein), the disclosures of which are incorporated herein by reference in their entireties and the groups A and L are as defined in the first, second and third aspects above (including embodiments thereof herein).

In an eleventh aspect, provided is a compound of Formula (III):

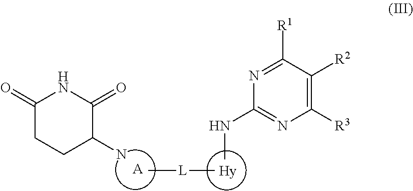

wherein ring A is as defined in the first, second or third aspect above (and any embodiments thereof disclosed herein) or

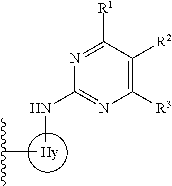

is as defined in the first, second or third or tenth aspect above and L is a linker illustrated in Table 1A below.

In a twelfth aspect, provided is a method of degrading CDK2 via ubiquitin proteosome pathway which method comprises contacting CDK2 protein with a compound of Formula (IV):

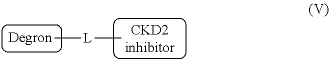

wherein:
Degron is an E3 ligase ligand; and
L is —Z¹-Z²—Z³—Z⁴-Z⁵—Z⁶— where —Z¹—, —Z²—, —Z³—, —Z⁴—, —Z⁵—, and —Z⁶— are as described in the first aspect.

In a thirteenth aspect, provided is a method of degrading CDK2 protein via ubiquitin proteosome pathway which method comprises attaching an E3 ligase ligand and a CDK2 inhibitor with a linker of formula:

—Z¹-Z²—Z³—Z⁴-Z⁵—Z⁶—

where —Z¹—, —Z²—, —Z³—, —Z⁴—, —Z⁵—, and —Z⁶— are as described in the first aspect.

### BRIEF DESCRIPTION OF THE DRAWINGS

FIG. 1. shows a dose-response curve of Compound 1 in Compound Table I in cellular CDK2 HTRF assay described in Biological Example 3.

FIG. 2. shows selective degradation of of CDK2 relative of CDK1, CDK4, CDK5 or cyclin E1 by Compound 1 in Compound Table I in both CDK2-dependent OVCAR3 and non CDK2-dependent HEK293 cells; and lack of RB phosphorylation at S780 and S807/7811 in OVCAR3 cells but not in HEK293 cells.

### DETAILED DESCRIPTION

### Definitions

Unless otherwise stated, the following terms used in the specification and claims are defined for the purposes of this Application and have the following meaning:

“Alkyl” means a linear saturated monovalent hydrocarbon radical of one to six carbon atoms or a branched saturated monovalent hydrocarbon radical of three to six carbon atoms, e.g., methyl, ethyl, propyl, 2-propyl, butyl, pentyl, and the like. It will be recognized by a person skilled in the art that the term “alkyl” may include “alkylene” groups.

“Alkylene” means a linear saturated divalent hydrocarbon radical of one to six carbon atoms or a branched saturated divalent hydrocarbon radical of three to six carbon atoms unless otherwise stated e.g., methylene, ethylene, propylene, 1-methylpropylene, 2-methylpropylene, butylene, pentylene, and the like.

“Alkenyl” means a linear unsaturated monovalent hydrocarbon radical of two to six carbon atoms or a branched saturated monovalent hydrocarbon radical of three to six carbon atoms containing a double bond, e.g., ethenyl, propenyl, 2-propenyl, butenyl, pentenyl, and the like.

“Alkynyl” means a linear unsaturated monovalent hydrocarbon radical of two to six carbon atoms or a branched saturated monovalent hydrocarbon radical of three to six carbon atom containing a triple bond, e.g., ethynyl, propynyl, and the like.

“Alkynylene” means a linear unsaturated divalent hydrocarbon radical of two to six carbon atoms or a branched saturated monovalent hydrocarbon radical of three to six carbon atom containing a triple bond, e.g.,

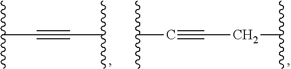

and the like.

“Alkylsulfonyl” means a —SO₂R^{z} radical where R^{z} is alkyl as defined above, e.g., methylsulfonyl, ethylsulfonyl, and the like.

“Alkylthio” means a —SR^{z} radical where R^{z} is alkyl as defined above, e.g., methylthio, ethylthio, and the like.

“Alkoxy” means a —OR^{z} radical where R^{z} is alkyl as defined above, e.g., methoxy, ethoxy, propoxy, or 2-propoxy, n-, iso-, or tert-butoxy, and the like.

“Alkoxyalkyl” means a linear monovalent hydrocarbon radical of one to six carbon atoms or a branched monovalent hydrocarbon radical of three to six carbons substituted with at least one alkoxy group, such as one or two alkoxy groups, as defined above, e.g., 2-methoxyethyl, 1-, 2-, or 3-methoxypropyl, 2-ethoxyethyl, and the like.

“Alkoxycarbonyl” means a —C(O)OR^{z} radical where R^{z} is alkyl as defined above, e.g., methoxycarbonyl, ethoxycarbonyl, and the like.

“Alkoxycarbonyloxy” means a —OC(O)OR^{z} radical where R^{z} is alkyl as defined above, e.g., methoxycarbonyloxy, ethoxycarbonyloxy, tert-butylcarbonyloxy, and the like.

“Alkoxycarbonylamino” means a —NR^{z}′C(O)OR^{z} radical where R^{z} is alkyl and R^{z}′ is H or alkyl, as defined above, e.g., methoxycarbonylamino, ethoxycarbonylamino, and the like.

“Acyl” means a —C(O)R^{z} radical where R^{z} is alkyl, haloalkyl, cycloalkyl, optionally substituted phenyl, optionally substituted heteroaryl, or optionally substituted heterocyclyl, as defined herein, e.g., methylcarbonyl, ethylcarbonyl, benzoyl, trifluoromethylcarbonyl, cyclopropylcarbonyl, and the like. When R^{z} is alkyl, acyl is also referred to herein as alkylcarbonyl.

“Amino” means a —NR^{z}′R^{z}″ radical where R^{z}′ and R^{z}″ are independently hydrogen, alkyl, haloalkyl, hydroxyalkyl, alkoxyalkyl, or alkylcarbonyl, each as defined herein, e.g., methylamino, hydroxyethylamino, and the like. When R^{z}′ is H and R″ is other than hydrogen, amino may also be referred to herein as monosubstituted amino. When R^{z}′ and R^{z}″ are other than hydrogen, amino may also be referred to herein as disubstituted amino. When R^{z}′ is H and R^{z}″ is alkyl, amino may also be referred to herein as alkylamino. When R^{z}′ and R^{z}″ are both alkyl, amino may also be referred to herein as dialkylamino.

“Aminocarbonyl” means a —CONR^{z}′R^{z}″ radical where R^{z}′ and R^{z}″ are independently hydrogen, alkyl, cycloalkyl which is optionally substituted with one, two, or three substituents independently selected from alkyl, halo, hydroxy, alkoxy, or cyano, haloalkyl, hydroxyalkyl, alkoxyalkyl, and alkylcarbonyl, each as defined herein, e.g., aminocarbonyl, methylaminocarbonyl, dimethylaminocarbonyl, and the like.

“Aminocarbonylalkyl” means a -(alkylene)-CONR^{z}′R^{z}″ radical where R^{z}′ and R^{z}″ are independently hydrogen, alkyl, cycloalkyl which is optionally substituted with one, two, or three substituents independently selected from alkyl, halo, hydroxy, alkoxy, cyano, haloalkyl, hydroxyalkyl, alkoxyalkyl, and alkylcarbonyl, each as defined herein.

“Aminosulfonyl” means a —SO₂NR^{z}′R^{z}″ radical where R^{z}′ and R^{z}″ are independently hydrogen, alkyl, cycloalkyl which is optionally substituted with one, two, or three substituents independently selected from alkyl, halo, hydroxy, alkoxy, cyano, haloalkyl, hydroxyalkyl, alkoxyalkyl, and alkylcarbonyl, each as defined herein, e.g., aminosulfonyl, methylaminosulfonyl, dimethylaminosulfonyl, and the like.

“Aminoalkyl” means a linear monovalent hydrocarbon radical of one to six carbon atoms or a branched monovalent hydrocarbon radical of three to six carbons substituted with —NR^{z}′R^{z}″ where R^{z}′ and R^{z}″ are independently hydrogen, alkyl, deuteroalkyl, cycloalkyl, cycloalkylalkyl (wherein cycloalkyl and cycloalkyl ring in cycloalkylalkyl is optionally substituted with one, two, or three substituents independently selected from alkyl, hydroxyalkyl, haloalkyl, halo, hydroxy, alkoxy, —NH₂, alkylamino, dialkylamino, alkylsulfonyl, alkoxycarbonylamino, and cyano), haloalkyl, hydroxyalkyl, alkoxyalkyl, aminocarbonylalkyl, haloalkoxyalkyl, alkylsulfonylalkyl, alkylcarbonyl, optionally substituted aryl, optionally substituted heteroaryl, optionally substituted heteroaralkyl, optionally substituted heterocyclyl, or optionally substituted heterocyclylalkyl, each as defined herein, e.g., aminomethyl, aminoethyl, methylaminomethyl, and the like.

“(Amino)deuteroalkyl” means a linear monovalent hydrocarbon radical of one to six carbon atoms or a branched monovalent hydrocarbon radical of three to six carbons substituted with one or two deuterium and —NR^{z}′R^{z}″ where R^{z}′ and R^{z}″ are independently hydrogen, alkyl, haloalkyl, hydroxyalkyl, alkoxyalkyl, or alkylcarbonyl, each as defined herein, e.g., aminomethyl (where one or two of the hydrogen in “methyl” is replaced with one or two deuterium, respectively), aminoethyl (where one or two of the hydrogen in “ethyl” is replaced with one or two deuterium, respectively), methylamino-C(H)(D)-, methylamino-CD₂-, and the like.

“Aryl” means a monovalent monocyclic or bicyclic aromatic hydrocarbon radical of 6 to 10 ring atoms e.g., phenyl or naphthyl.

“Aralkyl” means a -(alkylene)-R^{z} radical where R^{z} is aryl as defined above. Examples include, but are not limited to, benzyl, phenethyl, and the like.

“Bicyclic cycloalkyl” means a saturated monovalent fused bicyclic hydrocarbon radical of six to ten carbon atoms where the two rings are fused at two adjacent carbon ring atoms. Unless otherwise stated, bicyclic cycloalkyl is optionally substituted with one or two substituents independently selected from deuterium, alkyl, halo, haloalkyl, alkoxy, hydroxy, and cyano. Examples include, but are not limited to, bicyclo[3.1.0]hexan-6-yl, and the like.

“Bridged cycloalkyl” means a saturated monocyclic ring having 5 to 8 ring carbon ring atoms in which two non-adjacent ring atoms are linked by a (CR^{z}R^{z}′)n group where n is an integer selected from 1 to 3 inclusive and R^{z} and R^{z}′ are independently H or methyl (also may be referred to herein as “bridging” group). Unless otherwise stated, bridged cycloalkyl is optionally substituted with one or two substituents independently selected from deuterium, alkyl, halo, haloalkyl, alkoxy, hydroxy, and cyano. Examples include, but are not limited to, bicyclo[1.1.1]pentyl, bicyclo[2.1.1]hexyl, bicyclo[2.2.2]-octyl, and the like.

“Bicyclic heterocyclyl” means a saturated or unsaturated monovalent bicyclic group of 9 to 12 ring atoms in which one or two ring atoms are heteroatom independently selected from N, O, and S(O)ₙ, where n is an integer selected from 0 to 2, the remaining ring atoms being C, unless stated otherwise. Additionally, one or two ring carbon atoms of the bicyclic heterocyclyl can optionally be replaced by a —CO— group. More specifically the term bicyclic heterocyclyl includes, but is not limited to, hexahydrofuro[3,2-b]furanyl, and the like. When the heterocyclyl ring is unsaturated it can contain one or two ring double bonds provided that the ring is not aromatic.

“Bicyclic heterocyclylene” means a saturated or unsaturated divalent bicyclic group of 9 to 12 ring atoms in which one or two ring atoms are heteroatom independently selected from N, O, and S(O)ₙ, where n is an integer selected from 0 to 2 inclusive, the remaining ring atoms being C, unless stated otherwise. Additionally, one or two ring carbon atoms of the bicyclic heterocyclylene ring can optionally be replaced by a —CO— group. More specifically the term bicyclic heterocyclylene includes, but is not limited to, hexahydrofuro[3,2-b]furan-3,6-diyl, and the like. When the heterocyclylene ring is unsaturated it can contain one or two ring double bonds provided that the ring is not aromatic.

“Bridged heterocyclyl” means a saturated monovalent monocyclic ring having 5 to 7 ring carbon ring atoms in which two non-adjacent ring atoms are linked by a (CR^{z}R^{z}′)n group where n is 1 to 3 and R^{z} and R^{z}′ are independently H or methyl (also may be referred to herein as “bridging” group) and further wherein one or two ring carbon atoms, including an atom in the bridging group, is replaced by a heteroatom selected from N, O, and S(O)ₙ, where n is an integer selected from 0 to 2 inclusive. Bridged heterocyclyl is optionally substituted with one or two substituents independently selected from alkyl, halo, alkoxy, hydroxy, and cyano. Examples include, but are not limited to, 2-azabicyclo[2.2.2]octyl, quinuclidinyl, 7-oxabicyclo[2.2.1]heptyl, and the like.

“Bridged heterocyclylene” means a saturated divalent monocyclic ring having 5 to 7 ring carbon ring atoms in which two non-adjacent ring atoms are linked by a (CR^{z}R^{z}′). group where n is an integer selected from 1 to 3 inclusive and R^{z} and R^{z}′ are independently H or methyl (also may be referred to herein as “bridging” group) and further wherein one or two ring carbon atoms, including an atom in the bridging group, is replaced by a heteroatom selected from N, O, and S(O)ₙ, where n is an integer selected from 0 to 2 inclusive. Bridged heterocyclylene is optionally substituted with one or two substituents independently selected from alkyl, halo, alkoxy, hydroxy, and cyano unless stated otherwise. Examples include, but are not limited to, 3,8-diazabicyclo[3.2.1]octa-3,8-diyl, and the like.

“Cycloalkyl” means a monocyclic saturated monovalent hydrocarbon radical of three to ten carbon atoms. Examples include, but are not limited to, cyclopropyl, cyclobutyl, cyclopentyl, cyclohexyl, and the like.

“Cycloalkylalkyl” means an -(alkylene)-R^{z} radical where R^{z} is cycloalkyl as defined above. Examples include, but are not limited to, cyclopropylmethyl cyclobutylethyl, cyclopentylmethyl, cyclohexylmethyl, and the like.

“Cycloalkylene” means a divalent saturated hydrocarbon radical of three to six carbon atoms, otherwise e.g., 1,1-cyclopropylene, 1,1-cyclobutylene, 1,4-cyclohexylene, and the like.

“Cyanoalkyl” means a linear monovalent hydrocarbon radical of one to six carbon atoms or a branched monovalent hydrocarbon radical of three to six carbons substituted with cyano e.g., cyanomethyl, cyanoethyl, and the like.

“Carboxy” means —COOH.

“Cyclylaminylene” means a saturated divalent monocyclic ring of 4 to 8 ring atoms in which one ring atom is nitrogen, the remaining ring atoms being C. More specifically, the term cyclylaminyl includes, but is not limited to, pyrrolidinylene, piperidinylene, homopiperidinylene, and the like.

“Deuterium” mean refers to ²H or D.

“Deuteroalkyl” mean alkyl as defined above, which is substituted with one, two, or three deuterium.

“Fused heterocyclyl” as used herein, means a saturated monovalent monocyclic ring of 4 to 7 ring atoms having from one to three heteroatoms independently selected from N, O, and S and the remaining ring atoms being carbon, and further wherein two adjacent ring atoms of the heterocycloalkyl ring is fused to two adjacent ring members of a phenyl or a five or six membered heteroaryl, each as defined herein, unless stated otherwise. The nitrogen and sulfur atoms are optionally oxidized, and the nitrogen atom(s) are optionally quaternized and one or two carbon atoms of the fused ring atoms in the saturated monocyclic ring includes the two common ring vertices shared with the fused phenyl or five or six membered heteroaryl. The fused heterocyclyl can be attached at any atom of the ring. Non limiting examples of the fused heterocycloalkyl include 2,3-dihydrobenzo[b][1,4]-dioxinyl, 2-oxabicyclo[3.1.0]hexanyl, indolin-2-one-1-yl, indolinyl, and the like.

“Fused heterocyclylene” as used herein, means a saturated divalent monocyclic ring of 4 to 7 ring atoms having from one to three heteroatoms independently selected from N, O, and S and the remaining ring atoms being carbon, and further wherein two adjacent ring atoms of the heterocycloalkyl ring is fused to two adjacent ring members of a phenyl or a five or six membered heteroaryl, each as defined herein, unless stated otherwise. The nitrogen and sulfur atoms are optionally oxidized, and the nitrogen atom(s) are optionally quaternized and one or two carbon atoms of the fused ring atoms in the saturated monocyclic ring includes the two common ring vertices shared with the fused phenyl or five or six membered heteroaryl. The fused heterocyclylene can be attached at any two atoms of the ring.

“Halo” means fluoro, chloro, bromo, or iodo, preferably fluoro or chloro.

“Haloalkyl” means alkyl radical as defined above, which is substituted with one or more halogen atoms, e.g., one to five halogen atoms, such as fluorine or chlorine, including those substituted with different halogens, e.g., —CH₂Cl, —CF₃, —CHF₂, —CH₂CF₃, —CF₂CF₃, —CF(CH₃)₂, and the like. When the alkyl is substituted with only fluoro, it can be referred to in this Application as fluoroalkyl.

“Haloalkoxy” means a —OR^{z} radical where R^{z} is haloalkyl as defined above e.g., —OCF₃, —OCHF₂, and the like. When R^{z} is haloalkyl where the alkyl is substituted with only fluoro, it is referred to in this Application as fluoroalkoxy.

“Hydroxyalkyl” means a linear monovalent hydrocarbon radical of one to six carbon atoms or a branched monovalent hydrocarbon radical of three to six carbons substituted with one or two hydroxy groups, provided that if two hydroxy groups are present, they are not both present on the same carbon atom. Representative examples include, but are not limited to, hydroxymethyl, 2-hydroxy-ethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl and 2-(hydroxymethyl)-3-hydroxypropyl, preferably 2-hydroxyethyl, 2,3-dihydroxypropyl, and 1-(hydroxymethyl)-2-hydroxyethyl.

“Heteroaryl” means a monovalent monocyclic or bicyclic aromatic radical of 5 to 10 ring atoms, unless otherwise stated, where one or more, (in one embodiment, one, two, or three), ring atoms are heteroatom selected from N. O, and S, the remaining ring atoms being carbon. Representative examples include, but are not limited to, pyrrolyl, thienyl, thiazolyl, imidazolyl, furanyl, indolyl, isoindolyl, oxazolyl, isoxazolyl, benzothiazolyl, benzoxazolyl, quinolinyl, isoquinolinyl, pyridinyl, pyrimidinyl, pyrazinyl, pyridazinyl, triazolyl, tetrazolyl, and the like. As defined herein, the terms “heteroaryl” and “aryl” are mutually exclusive. When the heteroaryl ring contains 5- or 6 ring atoms and is a monocyclic ring, it is also referred to herein as 5- or 6-membered monocyclic heteroaryl. When the heteroaryl ring contains 9- or 10 ring atoms and is a bicyclic ring, it is also referred to herein as 9- or 10-membered fused bicyclic heteroaryl. “Heteroarylene” means a divalent heteroaryl radical as defined above. Representative examples include, but are not limited to, benzimidazoldiyl e.g., benzimidazole-1,5-diyl, and the like. When the heteroarylene is a monocyclic ring, it is also referred to herein as monocyclic heteroarylene. When the heteroarylene ring contains 5- or 6 ring atoms and is a monocyclic ring and is also referred to herein as 5- or 6-membered monocyclic heteroarylene e.g., pyrazolyl-1,4-diyl.

“Heteroaralkyl” means a -(alkylene)-R^{z} radical where R^{z} is heteroaryl as defined above, e.g., pyridinylmethyl, and the like. When the heteroaryl ring in heteroaralkyl contains 5- or 6 ring atoms it is also referred to herein as 5- or 6-membered heteroaralkyl.

“Heterocyclyl” means a saturated or unsaturated monovalent monocyclic group of 4 to 8 ring atoms in which one or two ring atoms are heteroatom independently selected from N, O, and S(O)ₙ, where n is an integer selected from 0 to 2 inclusive, the remaining ring atoms being C, unless stated otherwise. Additionally, one or two ring carbon atoms in the heterocyclyl ring can optionally be replaced by a —CO— group. More specifically the term heterocyclyl includes, but is not limited to, pyrrolidino, piperidino, homopiperidino, 2-oxopyrrolidinyl, 2-oxopiperidinyl, morpholino, piperazino, tetrahydro-pyranyl, thiomorpholino, and the like. When the heterocyclyl ring is unsaturated it can contain one or two ring double bonds provided that the ring is not aromatic. When the heterocyclyl group contains at least one nitrogen atom, it is also referred to herein as heterocycloamino and is a subset of the heterocyclyl group.

“Heterocyclylalkyl” or “heterocycloalkyl” means a -(alkylene)-R^{z} radical where R^{z} is heterocyclyl ring as defined above e.g., tetraydrofuranylmethyl, piperazinylmethyl, morpholinylethyl, and the like.

“Heterocyclylene” means a saturated divalent monocyclic group of 4 to 6 ring atoms in which one or two ring atoms are heteroatom independently selected from N, O, and S(O)ₙ, where n is an integer selected from 0 to 2 inclusive, the remaining ring atoms being C, unless stated otherwise. Additionally, one or two ring carbon atoms in the heterocyclylene ring can optionally be replaced by a —CO— group. More specifically, the term heterocyclylene includes, but is not limited to,

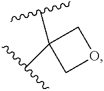 [SMILES: *C1(*)COC1]

piperidin-1,4-diyl, azetidin-1,3-diyl, and the like.

“Phenylene” refers to divalent phenyl.

The term “oxo,” as used herein, alone or in combination, refers to ═(O).

“Optionally substituted aryl” means aryl as defined above, that is optionally substituted with one, two, or three substituents independently selected from alkyl, hydroxyl, cycloalkyl, carboxy, alkoxycarbonyl, hydroxy, alkoxy, alkylthio, alkylsulfonyl, amino, alkylamino, dialkylamino, halo, haloalkyl, haloalkoxy, and cyano.

“Optionally substituted aralkyl” means -(alkylene)-R^{z} where R^{z} is optionally substituted aryl as defined above.

“Optionally substituted heteroaryl” means heteroaryl as defined above that is optionally substituted with one, two, or three substituents independently selected from alkyl, alkylthio, alkylsulfonyl, hydroxyl, cycloalkyl, carboxy, alkoxycarbonyl, hydroxy, alkoxy, halo, haloalkyl, haloalkoxy, amino, alkylamino, dialkylamino, and cyano.

“Optionally substituted heteroaralkyl” means -(alkylene)-R^{z} where R^{z} is optionally substituted heteroaryl as defined above.

“Optionally substituted heterocyclyl” means heterocyclyl as defined above that is optionally substituted with one, two, or three substituents independently selected from alkyl, alkylthio, alkylsulfonyl, alkylcarbonyl, hydroxyl, cycloalkyl, cycloalkylalkyl, carboxy, alkoxycarbonyl, hydroxy, hydroxyalkyl, alkoxy, alkoxyalkyl, aminoalkyl, cyanoalkyl, halo, haloalkyl, haloalkoxy, and cyano, unless stated otherwise.

“Optionally substituted heterocyclylalkyl” means -(alkylene)-R^{z} where R^{z} is optionally substituted heterocyclyl as defined above.

The present disclosure also includes protected derivatives of compounds of Formula (IA′), (IA), or (I). For example, when compounds of Formula (IA′), (IA), or (I) contain groups such as hydroxy, carboxy, or any group containing a nitrogen atom(s), these groups can be protected with suitable protecting groups. A comprehensive list of suitable protective groups can be found in T. W. Greene, Protective Groups in Organic Synthesis, 5^{th} Ed., John Wiley & Sons, Inc. (2014), the disclosure of which is incorporated herein by reference in its entirety. The protected derivatives of compounds of the present disclosure can be prepared by methods well known in the art.

The present disclosure also includes polymorphic forms and deuterated forms of the compound of Formula (IA′), (IA) or (I) or a pharmaceutically acceptable salt thereof.

The term “prodrug” refers to a compound that is made more active in vivo. Certain compounds Formula (IA′), (IA) or (I) may also exist as prodrugs, as described in Hydrolysis in Drug and Prodrug Metabolism: Chemistry, Biochemistry, and Enzymology (Testa, Bernard and Mayer, Joachim M. Wiley-VHCA, Zurich, Switzerland 2003). Prodrugs of the compounds described herein are structurally modified forms of the compound that readily undergo chemical changes under physiological conditions to provide the active compound. Prodrugs are often useful because, in some situations, they may be easier to administer than the compound, or parent drug. They may, for instance, be bioavailable by oral administration whereas the parent drug is not. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound.

A “pharmaceutically acceptable salt” of a compound means a salt that is pharmaceutically acceptable and that possesses the desired pharmacological activity of the parent compound. Such salts include:
acid addition salts, formed with inorganic acids such as hydrochloric acid, hydrobromic acid, sulfuric acid, nitric acid, phosphoric acid, and the like; or formed with organic acids such as formic acid, acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or
salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. It is understood that the pharmaceutically acceptable salts are non-toxic. Additional information on suitable pharmaceutically acceptable salts can be found in Remington's Pharmaceutical Sciences, 17th ed., Mack Publishing Company, Easton, PA, 1985, which is incorporated herein by reference in its entirety.

The compounds of Formula (IA′), (IA), or (I) may have asymmetric centers. Compounds of Formula (IA′), (IA,) or (I) containing an asymmetrically substituted atom may be isolated in optically active or racemic forms. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. All chiral, diastereomeric, all mixtures of chiral or diastereomeric forms, and racemic forms are within the scope of this disclosure, unless the specific stereochemistry or isomeric form is specifically indicated. It will also be understood by a person of ordinary skill in the art that when a compound is denoted as (R) stereoisomer, it may contain the corresponding (S) stereoisomer as an impurity and vice versa.

Certain compounds of Formula (IA′), (IA), or (I) can exist as tautomers and/or geometric isomers. All possible tautomers and cis and trans isomers, as individual forms and mixtures thereof are within the scope of this disclosure. Additionally, as used herein the term alkyl includes all the possible isomeric forms of said alkyl group albeit only a few examples are set forth. Furthermore, when the cyclic groups such as aryl is substituted, it includes all the positional isomers albeit only a few examples are set forth. Furthermore, all hydrates of a compound of Formula (IA′), (IA), or (I) are within the scope of this disclosure.

The compounds of Formula (IA′), (IA), or (I) may also contain unnatural amounts of isotopes at one or more of the atoms that constitute such compounds. Unnatural amounts of an isotope may be defined as ranging from the amount found in nature to an amount 100% of the atom in question. that differ only in the presence of one or more isotopically enriched atoms. Exemplary isotopes that can be incorporated into compounds of the present disclosure, such as a compound of Formula (IA′), (IA), or (I) (and any embodiment thereof disclosed herein including specific compounds) include isotopes of hydrogen, carbon, nitrogen, oxygen, phosphorus, sulfur, fluorine, chlorine, and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I, and ¹²⁵I, respectively. Isotopically labeled compounds (e.g., those labeled with ³H and ¹⁴C) can be useful in compound or substrate tissue distribution assays. Tritiated (i.e., ³H) and carbon-14 (i.e., ¹⁴C) isotopes can be useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements). In some embodiments, in compounds of Formula (IA′), (IA), or (I), including in Table 1 below one or more hydrogen atoms are replaced by ²H or ³H, or one or more carbon atoms are replaced by ¹³C- or ¹⁴C-enriched carbon. Positron emitting isotopes such as ¹⁵O, ¹³N, ¹¹C, and ¹⁵F are useful for positron emission tomography (PET) studies to examine substrate receptor occupancy. Isotopically labeled compounds can generally be prepared by following procedures analogous to those disclosed in the Schemes or in the Examples herein, by substituting an isotopically labeled reagent for a non-isotopically labeled reagent.

A “pharmaceutically acceptable carrier or excipient” means a carrier or an excipient that is useful in preparing a pharmaceutical composition that is generally safe, non-toxic and neither biologically nor otherwise undesirable, and includes a carrier or an excipient that is acceptable for veterinary use as well as human pharmaceutical use.

“A pharmaceutically acceptable carrier/excipient” as used in the specification and claims includes both one and more than one such excipient.

“Spiroheterocyclyl” means a saturated bicyclic monovalent ring having 6 to 10 ring atoms in which one, two, or three ring atoms are heteroatom selected from N, O, and S(O)n, where n is an integer selected from 0 to 2 inclusive, the remaining ring atoms being C and the rings are connected through only one atom, the connecting atom is also called the spiroatom, most often a quaternary carbon (“spiro carbon”). Spiroheterocyclyl is optionally substituted with one or two substituents independently selected from alkyl, halo, alkoxy, hydroxy, and cyano, unless otherwise stated. Representative examples include, but are not limited to, 2,6-diazaspiro-[3.3]heptanyl, 2,6-diazaspiro[3.4]octanyl, 2-azaspiro[3.4]octanyl, 2-azaspiro[3.5]-nonanyl, 2,7-diazaspiro[4.4]nonanyl, and the like.

“Spiro cycloalkylene” means a saturated bicyclic divalent hydrocarbon ring having 9 to 12 ring atoms wherein the rings are connected through only one atom, the connecting atom is also called the spiroatom, most often a quaternary carbon (“spiro carbon”). Spiro cycloalkylene is optionally substituted with one or two substituents independently selected from alkyl, halo, alkoxy, hydroxy, and cyano, unless stated otherwise. Representative examples include, but are not limited to, spiro[3,5]nonandiyl e.g., spiro[3.5]nonane-2,7-diyl, and the like.

“Spiro heterocyclylene” means a saturated bicyclic divalent ring having 6 to 10 ring atoms in which one, two, or three ring atoms are heteroatom selected from N, O, and S(O)ₙ, where n is an integer selected from 0 to 2 inclusive, the remaining ring atoms being C and the rings are connected through only one atom, the connecting atom is also called the spiroatom, most often a quaternary carbon (“spiro carbon”). Spiro heterocyclylene is optionally substituted with one or two substituents independently selected from alkyl, halo, alkoxy, hydroxy, and cyano, unless stated otherwise.

“11 to 13 membered spiro heterocyclylene” means a saturated bicyclic divalent ring having 11 to 13 ring atoms in which one, two, or three ring atoms are heteroatom(s) selected from N, O, and S(O)ₙ, where n is an integer selected from 0 to 2 inclusive, the remaining ring atoms being C and the rings are connected through only one atom, the connecting atom is also called the spiroatom, most often a quaternary carbon (“spiro carbon”). The 11 to 13 membered spiro heterocyclylene is optionally substituted with one or two substituents independently selected from alkyl, halo, alkoxy, hydroxy, and cyano, unless stated otherwise. Representative examples include, but are not limited to, diazaspiro[5.5]undecan-diyl, 1-oxa-diazaspiro[5.5]undecan-diyl, and the like.

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass ±10%, preferably ±5%, the recited value and the range is included.

The phrase optionally substituted aryl in the definition of R³ in Formula (IA′), (IA), or (I) (and similar phrases used to define other groups in Formula (IA′), (IA), or (I)) is intended to cover aryl that is unsubstituted and aryl that is substituted with substituents denoted in the definition thereof.

Certain structures provided herein are drawn with one or more floating substituents. Unless provided otherwise or otherwise clear from the context, the substituent(s) may be present on any atom of the ring to which it is attached, where chemically feasible and valency rules permitting.

For example, in the structure:

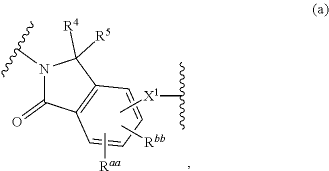

the R^{aa} substituent of R^{aa}, R^{bb} and X¹, and similarly the R^{bb} and X¹ substituents, can replace hydrogen of any CH that is part of the benzo portion of the bicyclic ring that is not already substituted with R^{bb} and X¹, and similarly R^{aa} and X¹ and R^{aa} and X^{bb} substituents respectively.

Additionally, as used throughout the application, including in the embodiments, when a group is drawn out as divalent, the left bond of the divalent group is attached to the group which is to its left in the remainder of the molecule, and the right bond of the divalent group is attached to the group which is to its right in the remainder of the molecule, For example, in the following divalent groups

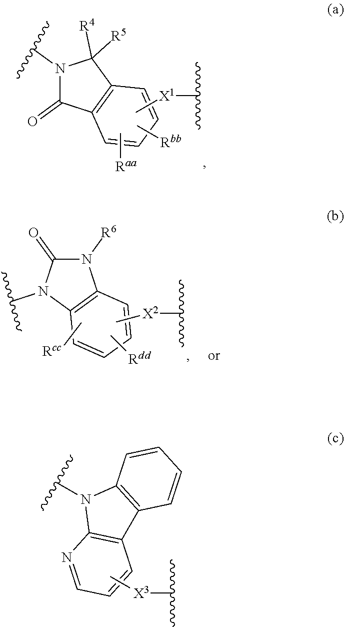

the

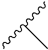

bond on the left of (a), (b) and (c) is attached to the following ring:

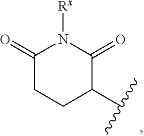

and the

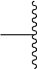

on the right side of (a), (b), and (c) (i.e., X¹, X², and X³) is attached to Z¹ of L of the following structure:

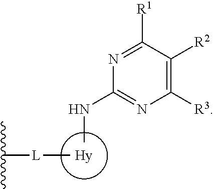

Similarly, for L i.e. —Z¹-Z²—Z³—Z⁴-Z⁵—Z⁶—, the left side in L (i.e., Z¹) is attached to X¹, X², X³, or X⁴ and Z⁶ is attached to an atom of Hy. For example, when L is a group of formula:

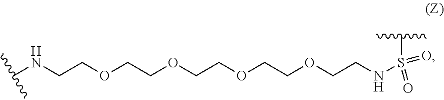 [SMILES: *NCCOCCOCCOCCOCCNS(*)(=O)=O]

and Degron is a group of formula (a), i.e.,

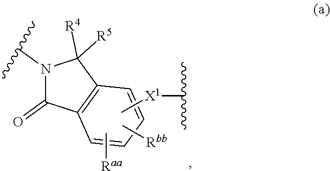

the left bond in (Z) (i.e., the —NH— group) is attached to X¹ and the right hand bond in (Z) (i.e., —SO₂—) is attached to an atom of the Hy

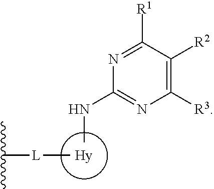

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “combination therapy” means the administration of two or more therapeutic agents to treat a disease or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

The term “patient” is generally synonymous with the term “subject” and includes all mammals including humans. Examples of patients include humans, livestock such as cows, goats, sheep, pigs, and rabbits, and companion animals such as dogs, cats, rabbits, and horses. Preferably, the patient is a human.

“Treating” or “treatment” of a disease includes:
(1) preventing the disease, i.e., causing the clinical symptoms of the disease not to develop in a mammal that may be exposed to or predisposed to the disease but does not yet experience or display symptoms of the disease;
(2) inhibiting the disease, i.e., delaying, arresting or reducing the development or severity of the disease or its clinical symptoms; or
(3) relieving the disease, i.e., causing regression of the disease or its clinical symptoms.

In one embodiment, treating or treatment of a disease includes inhibiting the disease, i.e., delaying, arresting or reducing the development or severity of the disease or its clinical symptoms; or relieving the disease, i.e., causing regression of the disease or its clinical symptoms.

A “therapeutically effective amount” means the amount of a compound of the present disclosure and/or a pharmaceutically acceptable salt thereof that, when administered to a patient for treating a disease, is sufficient to affect such treatment for the disease. The “therapeutically effective amount” will vary depending on the compound, the disease and its severity and the age, weight, etc., of the mammal to be treated.

The terms “inhibiting” and “reducing,” or any variation of these terms in relation of CDK2 and/or CDK1, includes any measurable decrease or complete inhibition to achieve a desired result.

For example, there may be a decrease of about, at most about, or at least about 5%, 10%, 15%, 20%, 25%, 30%, 35%, 40%, 45%, 50%, 55%, 60%, 65%, 70%, 75%, 80%, 85%, 90%, 95%, 99%, or more, or any range derivable therein, reduction of CDK2 and/or CDK1 activity respectively, compared to normal.

Representative compounds of the disclosure made are disclosed in Compound Table I below:

Compound Table I

| Cpd | | |
|---|---|---|
| # | Structure | Name |
| 1 | 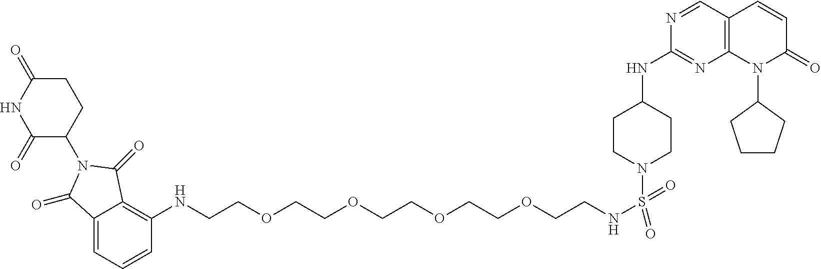 [SMILES: O=C1CCC(N2C(=O)c3cccc(NCCOCCOCCOCCOCCNS(=O)(=O)N4CCC(Nc5ncc6ccc(=O)n(C7CCCC7)c6n5)CC4)c3C2=O)C(=O)N1] | 4-((8-cyclopentyl-7-oxo-7,8-dihydro- pyrido[2,3-d]pyrimidin-2-yl)amino)-N- (14-((2-(2,6-dioxopiperidin-3-yl)-1,3- dioxoisoindolin-4-yl)amino)-3,6,9,12- tetraoxatetradecyl)piperidine-1- sulfonamide |
| 2 | 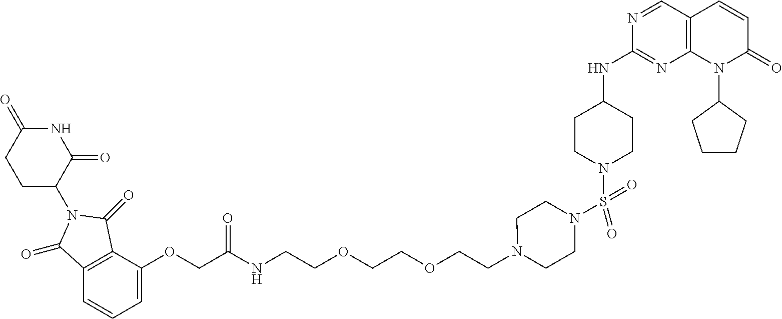 [SMILES: O=C(COc1cccc2c1C(=O)N(C1CCC(=O)NC1=O)C2=O)NCCOCCOCCN1CCN(S(=O)(=O)N2CCC(Nc3ncc4ccc(=O)n(C5CCCC5)c4n3)CC2)CC1] | N-(2-(2-(2-(4-((4-((8-cyclopentyl-7-oxo- 7,8-dihydropyrido[2,3-d]pyrimidin-2- yl)amino)-piperidin-1- yl)sulfonyl)piperazin-1-yl)- ethoxy)ethoxy)ethyl)-2-((2-(2,6-dioxo- piperidin-3-yl)-1,3-dioxoisoindolin-4-yl)- oxy)acetamide |
| 3 | 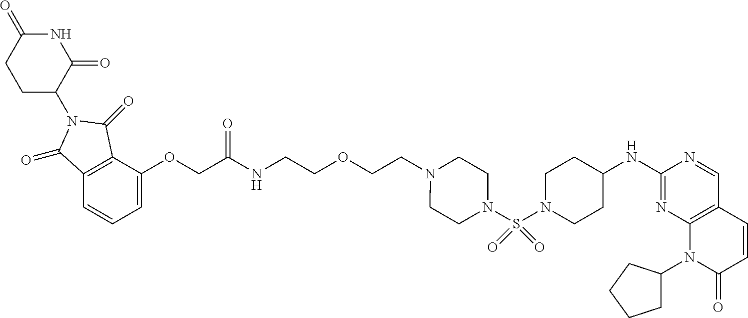 [SMILES: O=C(COc1cccc2c1C(=O)N(C1CCC(=O)NC1=O)C2=O)NCCOCCN1CCN(S(=O)(=O)N2CCC(Nc3ncc4ccc(=O)n(C5CCCC5)c4n3)CC2)CC1] | N-(2-(2-(4-((4-((8-cyclopentyl-7-oxo-7,8- dihydropyrido[2,3-d]pyrimidin-2- yl)amino)-piperidin-1- yl)sulfonyl)piperazin-1-yl)-ethoxy)ethyl)- 2-((2-(2,6-dioxopiperidin-3-yl)-1,3- dioxoisoindolin-4-yl)oxy)acetamide |
| 4 | 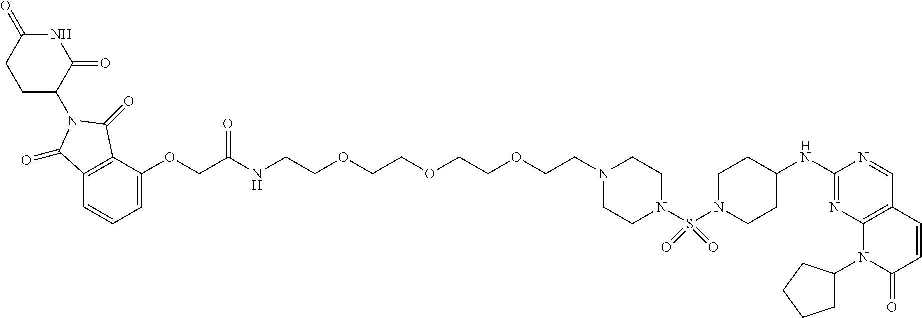 [SMILES: O=C(COc1cccc2c1C(=O)N(C1CCC(=O)NC1=O)C2=O)NCCOCCOCCOCCN1CCN(S(=O)(=O)N2CCC(Nc3ncc4ccc(=O)n(C5CCCC5)c4n3)CC2)CC1] | N-(2-(2-(2-(2-(4-((4-((8-cyclopentyl-7- ox0-7,8-dihydropyrido[2,3-d]pyrimidin-2- yl)-amino)piperidin-1- yl)sulfonyl)piperazin-1- yl)ethoxy)ethoxy)ethoxy)ethyl)-2-((2-(2,6- dioxopiperidin-3-yl)-1,3-dioxoisoindolin- 4-yl)oxy)acetamide |
| 5 | 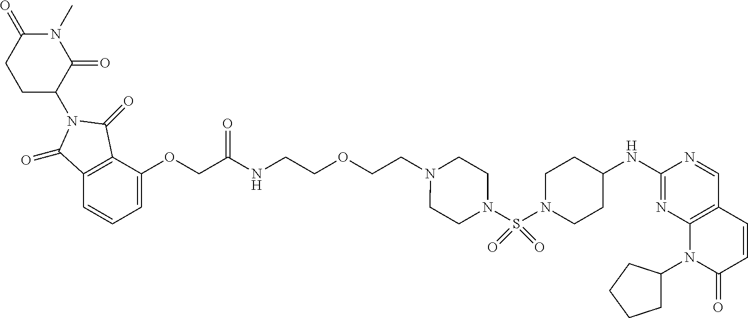 [SMILES: CN1C(=O)CCC(N2C(=O)c3cccc(OCC(=O)NCCOCCN4CCN(S(=O)(=O)N5CCC(Nc6ncc7ccc(=O)n(C8CCCC8)c7n6)CC5)CC4)c3C2=O)C1=O] | N-(2-(2-(4-((4-((8-cyclopentyl-7-oxo-7,8- dihydropyrido[2,3-d]pyrimidin-2- yl)amino)-piperidin-1- yl)sulfonyl)piperazin-1-yl)-ethoxy)ethyl)- 2-((2-(1-methyl-2,6-dioxo-piperidin-3-yl)- 1,3-dioxoisoindolin-4-yl)oxy)acetamide |
| 6 | 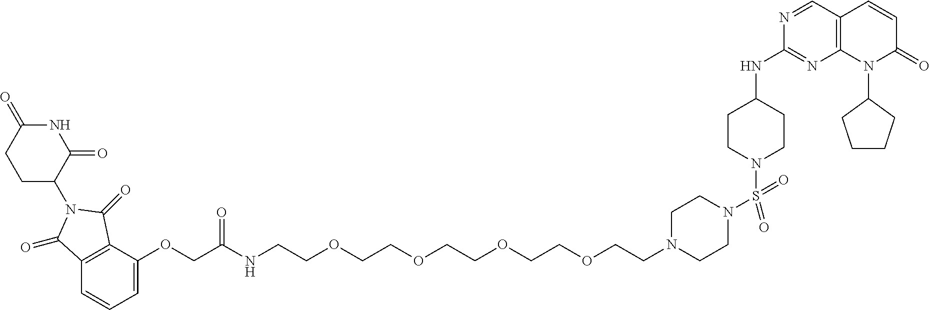 [SMILES: O=C(COc1cccc2c1C(=O)N(C1CCC(=O)NC1=O)C2=O)NCCOCCOCCOCCOCCN1CCN(S(=O)(=O)N2CCC(Nc3ncc4ccc(=O)n(C5CCCC5)c4n3)CC2)CC1] | N-(14-(4-((4-((8-cyclopentyl-7-oxo-7,8- dihydro-pyrido[2,3-d]pyrimidin-2- yl)amino)-piperidin-1- yl)sulfonyl)piperazin-1-yl)-3,6,9,12- tetraoxatetradecyl)-2-((2-(2,6- dioxopiperidin-3-yl)-1,3-dioxoisoindolin- 4-yl)oxy)acetamide |
| 7 | 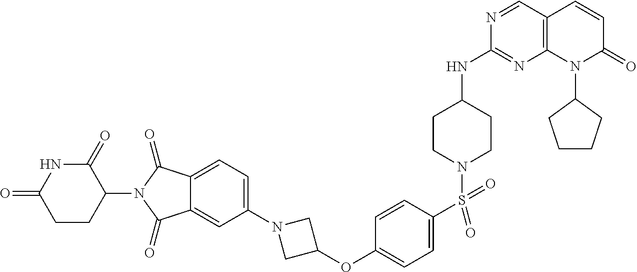 [SMILES: O=C1CCC(N2C(=O)c3ccc(N4CC(Oc5ccc(S(=O)(=O)N6CCC(Nc7ncc8ccc(=O)n(C9CCCC9)c8n7)CC6)cc5)C4)cc3C2=O)C(=O)N1] | 5-(3-(4-((4-((8-cyclopentyl-7-oxo-7,8- dihydro-pyrido[2,3-d]pyrimidin-2- yl)amino)piperidin-1- yl)sulfonyl)phenoxy)azetidin-1-yl)-2-(2,6- dioxopiperidin-3-yl)isoindoline-1,3-dione |
| 8 | 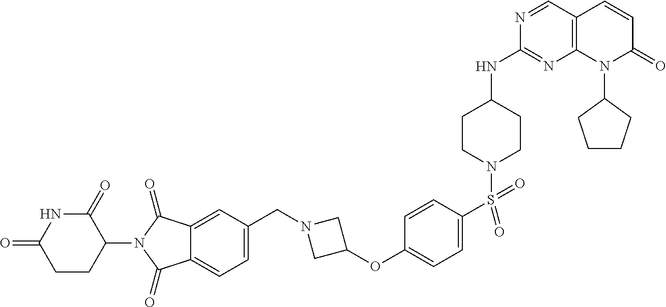 [SMILES: O=C1CCC(N2C(=O)c3ccc(CN4CC(Oc5ccc(S(=O)(=O)N6CCC(Nc7ncc8ccc(=O)n(C9CCCC9)c8n7)CC6)cc5)C4)cc3C2=O)C(=O)N1] | 5-((3-(4-((4-((8-cyclopentyl-7-oxo-7,8- dihydro-pyrido[2,3-d]pyrimidin-2- yl)amino)piperidin-1- yl)sulfonyl)phenoxy)azetidin-1- yl)methyl)-2-(2,6-dioxopiperidin-3- yl)isoindoline-1,3-dione |
| 9 | 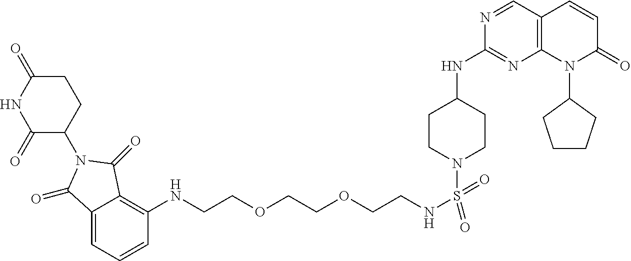 [SMILES: O=C1CCC(N2C(=O)c3cccc(NCCOCCOCCNS(=O)(=O)N4CCC(Nc5ncc6ccc(=O)n(C7CCCC7)c6n5)CC4)c3C2=O)C(=O)N1] | 4-((8-cyclopentyl-7-oxo-7,8- dihydropyrido[2,3-d]-pyrimidin-2- yl)amino)-N-(2-(2-(2-((2-(2,6- dioxopiperidin-3-yl)-1,3-dioxoisoindolin- 4-yl)- amino)ethoxy)ethoxy)ethyl)piperidine-1- sulfonamide |
| 10 | 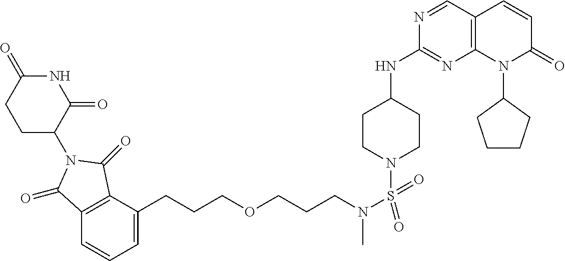 [SMILES: CN(CCCOCCCc1cccc2c1C(=O)N(C1CCC(=O)NC1=O)C2=O)S(=O)(=O)N1CCC(Nc2ncc3ccc(=O)n(C4CCCC4)c3n2)CC1] | 4-((8-cyclopentyl-7-oxo-7,8- dihydropyrido[2,3-d]-pyrimidin-2- yl)amino)-N-(3-(3-(2-(2,6-dioxo-piperidin- 3-yl)-1,3-dioxoisoindolin-4-yl)- propoxy)propyl)-N-methylpiperidine-1- sulfonamide |
| 11 | 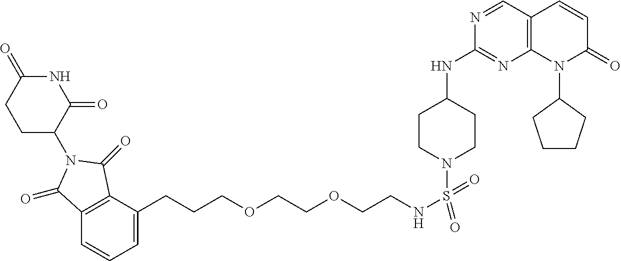 [SMILES: O=C1CCC(N2C(=O)c3cccc(CCCOCCOCCNS(=O)(=O)N4CCC(Nc5ncc6ccc(=O)n(C7CCCC7)c6n5)CC4)c3C2=O)C(=O)N1] | 4-((8-cyclopentyl-7-oxo-7,8- dihydropyrido[2,3-d]pyrimidin-2- yl)amino)-N-(2-(2-(3-(2-(2,6- dioxopiperidin-3-yl)-1,3-dioxoisoindolin- 4-yl)-propoxy)ethoxy)ethyl)piperidine-1- sulfonamide |
| 12 | 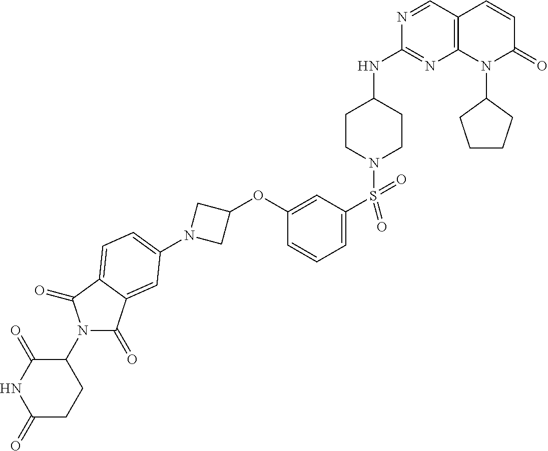 [SMILES: O=C1CCC(N2C(=O)c3ccc(N4CC(Oc5cccc(S(=O)(=O)N6CCC(Nc7ncc8ccc(=O)n(C9CCCC9)c8n7)CC6)c5)C4)cc3C2=O)C(=O)N1] | 5-(3-(3-((4-((8-cyclopentyl-7-oxo-7,8- dihydro-pyrido[2,3-d]pyrimidin-2- yl)amino)piperidin-1- yl)sulfonyl)phenoxy)azetidin-1-yl)-2-(2,6- dioxopiperidin-3-yl)isoindoline-1,3-dione |
| 13 | 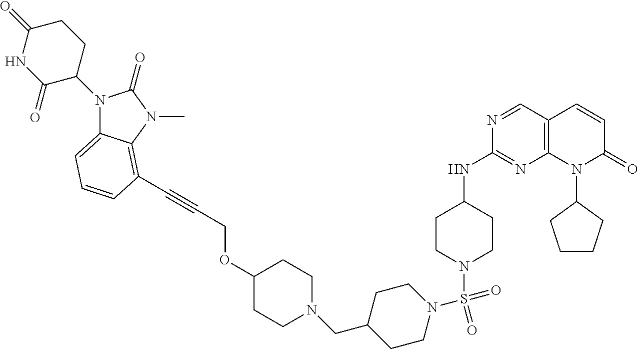 [SMILES: Cn1c(=O)n(C2CCC(=O)NC2=O)c2cccc(C#CCOC3CCN(CC4CCN(S(=O)(=O)N5CCC(Nc6ncc7ccc(=O)n(C8CCCC8)c7n6)CC5)CC4)CC3)c21] | 3-(4-(3-((1-((1-((4-((8-cyclopentyl-7-oxo- 7,8-dihydropyrido[2,3-d]pyrimidin-2- yl)amino)-piperidin-1- yl)sulfonyl)piperidin-4-yl)methyl)- piperidin-4-yl)oxy)prop-1-yn-1-yl)-3- methyl-2-oxo-2,3-dihydro-1H- benzo[d]imidazol-1-yl)-piperidine-2,6- dione |
| 14 | 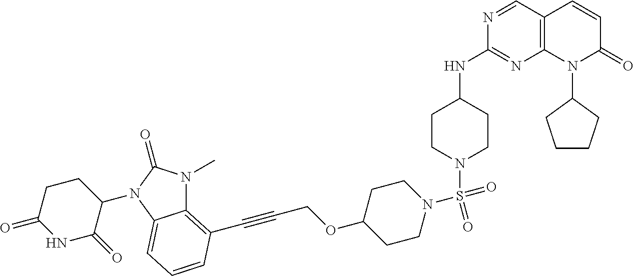 [SMILES: Cn1c(=O)n(C2CCC(=O)NC2=O)c2cccc(C#CCOC3CCN(S(=O)(=O)N4CCC(Nc5ncc6ccc(=O)n(C7CCCC7)c6n5)CC4)CC3)c21] | 3-(4-(3-((1-((4-((8-cyclopentyl-7-oxo-7,8- dihydropyrido[2,3-d]pyrimidin-2- yl)amino)-piperidin-1- yl)sulfonyl)piperidin-4-yl)oxy)prop-1-yn- 1-yl)-3-methyl-2-oxo-2,3-dihydro-1H- benzo[d]imidazol-1-yl)piperidine-2,6- dione |
| 15 | 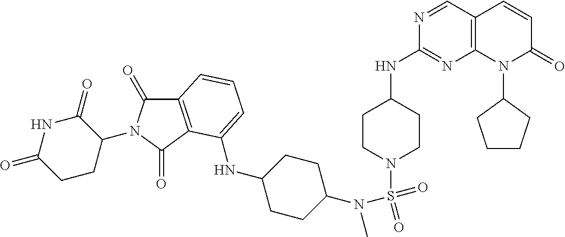 [SMILES: CN(C1CCC(Nc2cccc3c2C(=O)N(C2CCC(=O)NC2=O)C3=O)CC1)S(=O)(=O)N1CCC(Nc2ncc3ccc(=O)n(C4CCCC4)c3n2)CC1] | 4-((8-cyclopentyl-7-oxo-7,8- dihydropyrido[2,3-d]pyrimidin-2- yl)amino)-N-(4-((2-(2,6-dioxo-piperidin-3- yl)-1,3-dioxoisoindolin-4-yl)amino)- cyclohexyl)-N-methylpiperidine-1- sulfonamide |
| 16 | 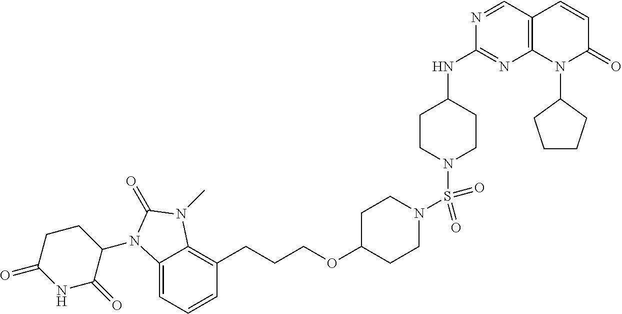 [SMILES: Cn1c(=O)n(C2CCC(=O)NC2=O)c2cccc(CCCOC3CCN(S(=O)(=O)N4CCC(Nc5ncc6ccc(=O)n(C7CCCC7)c6n5)CC4)CC3)c21] | 3-(4-(3-((1-((4-((8-cyclopentyl-7-oxo-7,8- dihydropyrido[2,3-d]pyrimidin-2- yl)amino)-piperidin-1- yl)sulfonyl)piperidin-4-yl)oxy)propyl)-3- methyl-2-oxo-2,3-dihydro-1H-benzo[d]- imidazol-1-yl)piperidine-2,6-dione |
| 17 | 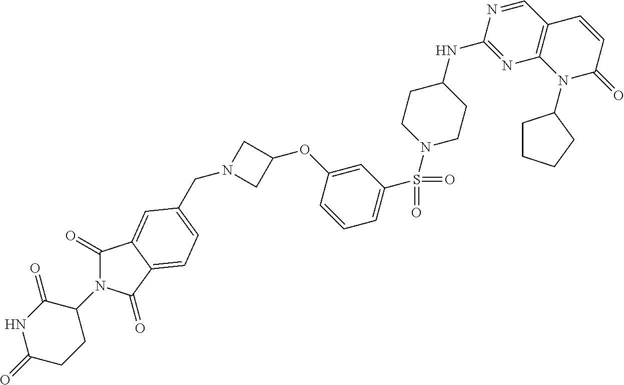 [SMILES: O=C1CCC(N2C(=O)c3ccc(CN4CC(Oc5cccc(S(=O)(=O)N6CCC(Nc7ncc8ccc(=O)n(C9CCCC9)c8n7)CC6)c5)C4)cc3C2=O)C(=O)N1] | 5-((3-(3-((4-((8-cyclopentyl-7-oxo-7,8- dihydro-pyrido[2,3-d]pyrimidin-2- yl)amino)piperidin-1- yl)sulfonyl)phenoxy)azetidin-1- yl)methyl)-2-(2,6-dioxopiperidin-3- yl)isoindoline-1,3-dione |
| 18 | 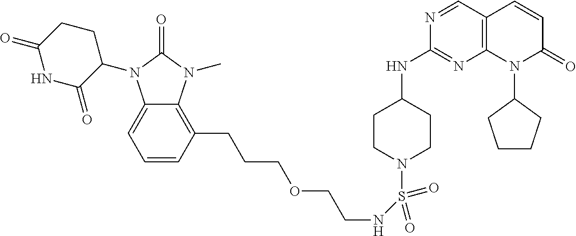 [SMILES: Cn1c(=O)n(C2CCC(=O)NC2=O)c2cccc(CCCOCCNS(=O)(=O)N3CCC(Nc4ncc5ccc(=O)n(C6CCCC6)c5n4)CC3)c21] | 4-((8-cyclopentyl-7-oxo-7,8- dihydropyrido[2,3-d]pyrimidin-2- yl)amino)-N-(2-(3-(1-(2,6-dioxopiperidin- 3-yl)-3-methyl-2-oxo-2,3-dihydro-1H- benzo[d]imidazol-4-yl)propoxy)- ethyl)piperidine-1-sulfonamide |
| 19 | 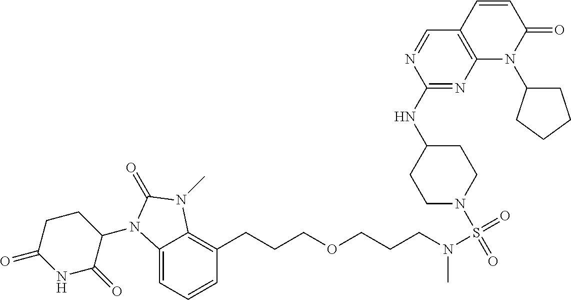 [SMILES: CN(CCCOCCCc1cccc2c1n(C)c(=O)n2C1CCC(=O)NC1=O)S(=O)(=O)N1CCC(Nc2ncc3ccc(=O)n(C4CCCC4)c3n2)CC1] | 4-((8-cyclopentyl-7-oxo-7,8- dihydropyrido[2,3-d]pyrimidin-2- yl)amino)-N-(3-(3-(1-(2,6-dioxo-piperidin- 3-yl)-3-methyl-2-oxo-2,3-dihydro-1H- benzo[d]imidazol-4-yl)propoxy)propyl)-N- methylpiperidine-1-sulfonamide |
| 20 | 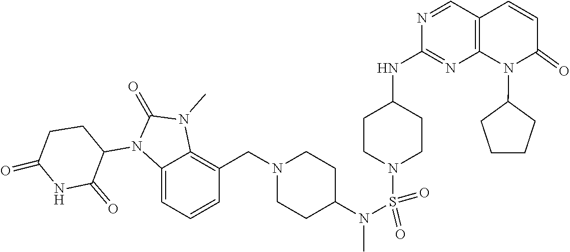 [SMILES: CN(C1CCN(Cc2cccc3c2n(C)c(=O)n3C2CCC(=O)NC2=O)CC1)S(=O)(=O)N1CCC(Nc2ncc3ccc(=O)n(C4CCCC4)c3n2)CC1] | 4-((8-cyclopentyl-7-oxo-7,8- dihydropyrido[2,3-d]pyrimidin-2- yl)amino)-N-(1-((1-(2,6-dioxo-piperidin-3- yl)-3-methyl-2-oxo-2,3-dihydro-1H- benzo[d]imidazol-4-yl)methyl)piperidin-4- yl)-N-methylpiperidine-1-sulfonamide |
| 21 | 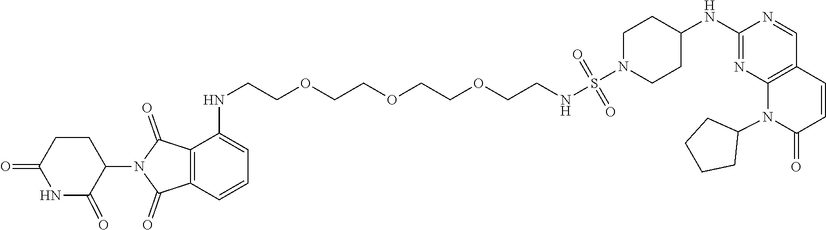 [SMILES: O=C1CCC(N2C(=O)c3cccc(NCCOCCOCCOCCNS(=O)(=O)N4CCC(Nc5ncc6ccc(=O)n(C7CCCC7)c6n5)CC4)c3C2=O)C(=O)N1] | 4-((8-cyclopentyl-7-oxo-7,8- dihydropyrido[2,3-d]pyrimidin-2- yl)amino)-N-(2-(2-(2-(2-((2-(2,6- dioxopiperidin-3-yl)-1,3-dioxoisoindolin- 4- yl)amino)ethoxy)ethoxy)ethoxy)ethyl) piperidine-1-sulfonamide |
| 22 | 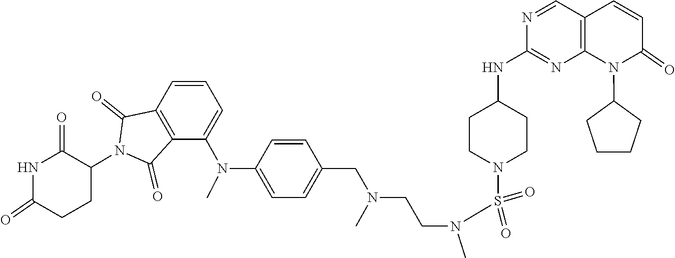 [SMILES: CN(CCN(C)S(=O)(=O)N1CCC(Nc2ncc3ccc(=O)n(C4CCCC4)c3n2)CC1)Cc1ccc(N(C)c2cccc3c2C(=O)N(C2CCC(=O)NC2=O)C3=O)cc1] | 4-((8-cyclopentyl-7-oxo-7,8- dihydropyrido[2,3-d]-pyrimidin-2- yl)amino)-N-(2-((4-((2-(2,6- dioxopiperidin-3-yl)-1,3-dioxoisoindolin- 4- yl)(methyl)amino)benzyl)(methyl)amino) ethyl)-N-methylpiperidine-1-sulfonamide |
| 23 | 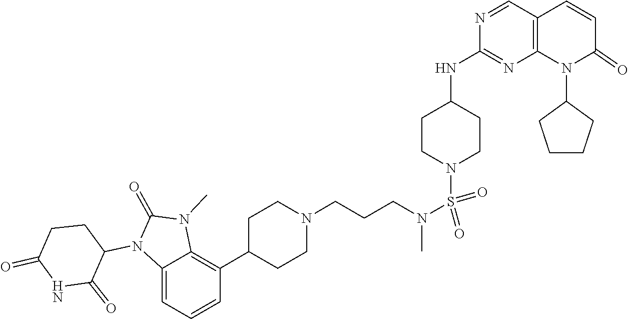 [SMILES: CN(CCCN1CCC(c2cccc3c2n(C)c(=O)n3C2CCC(=O)NC2=O)CC1)S(=O)(=O)N1CCC(Nc2ncc3ccc(=O)n(C4CCCC4)c3n2)CC1] | 4-((8-cyclopentyl-7-oxo-7,8- dihydropyrido[2,3-d]pyrimidin-2- yl)amino)-N-(3-(4-(1-(2,6-dioxo-piperidin- 3-yl)-3-methyl-2-oxo-2,3-dihydro-1H- benzo[d]imidazol-4-yl)piperidin-1- yl)propyl)-N-methylpiperidine-1- sulfonamide |
| 24 | 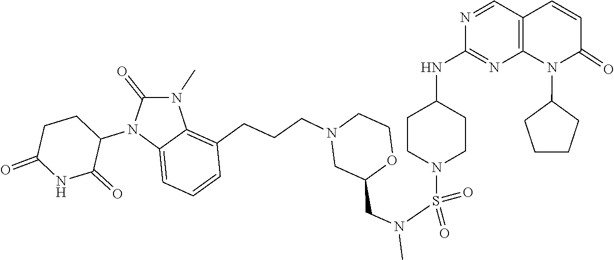 [SMILES: CN(C[C@H]1CN(CCCc2cccc3c2n(C)c(=O)n3C2CCC(=O)NC2=O)CCO1)S(=O)(=O)N1CCC(Nc2ncc3ccc(=O)n(C4CCCC4)c3n2)CC1] | 4-((8-cyclopentyl-7-oxo-7,8- dihydropyrido[2,3-d]pyrimidin-2- yl)amino)-N-(((2R)-4-(3-(1-(2,6- dioxopiperidin-3-yl)-3-methyl-2-oxo-2,3- dihydro-1H-benzo[d]imidazol-4- yl)propyl)morpholin-2-yl)methyl)-N- methylpiperidine-1-sulfonamide |
| 25 | 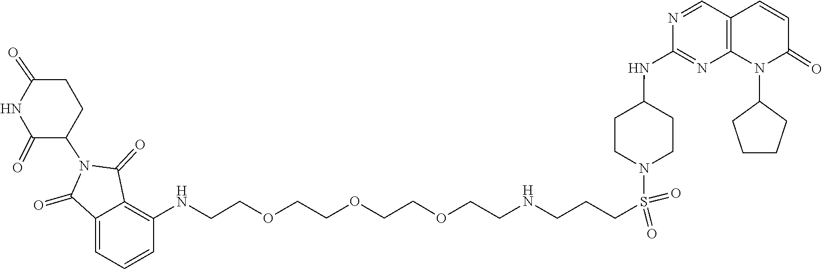 [SMILES: O=C1CCC(N2C(=O)c3cccc(NCCOCCOCCOCCNCCCS(=O)(=O)N4CCC(Nc5ncc6ccc(=O)n(C7CCCC7)c6n5)CC4)c3C2=O)C(=O)N1] | 4-((15-((4-((8-cyclopentyl-7-oxo-7,8- dihydro-pyrido[2,3-d]pyrimidin-2- yl)amino)piperidin-1-yl)sulfonyl)-3,6,9- trioxa-12-azapentadecyl)-amino)-2-(2,6- dioxopiperidin-3-yl)isoindoline-1,3-dione |
| 26 | 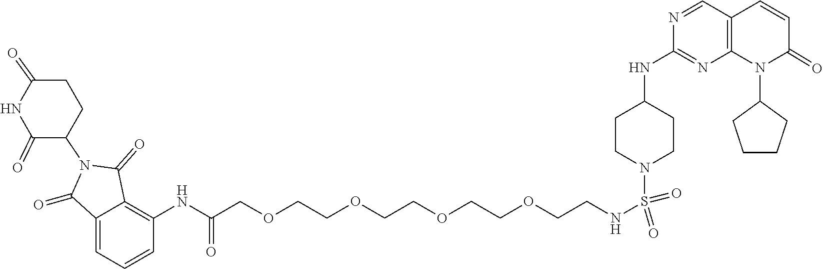 [SMILES: O=C1CCC(N2C(=O)c3cccc(NC(=O)COCCOCCOCCOCCNS(=O)(=O)N4CCC(Nc5ncc6ccc(=O)n(C7CCCC7)c6n5)CC4)c3C2=O)C(=O)N1] | 14-((4-((8-cyclopentyl-7-oxo-7,8-dihydro- pyrido[2,3-d]pyrimidin-2- yl)amino)piperidine)-1-sulfonamido)-N- (2-(2,6-dioxopiperidin-3-yl)-1,3- dioxoisoindolin-4-yl)-3,6,9,12- tetraoxatetradecan-amide |
| 27 | 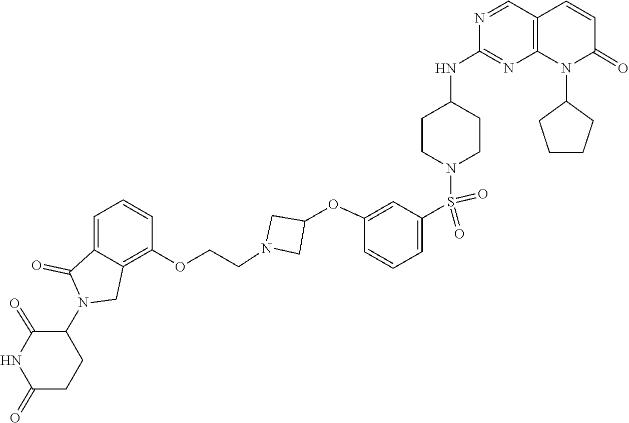 [SMILES: O=C1CCC(N2Cc3c(OCCN4CC(Oc5cccc(S(=O)(=O)N6CCC(Nc7ncc8ccc(=O)n(C9CCCC9)c8n7)CC6)c5)C4)cccc3C2=O)C(=O)N1] | 3-(4-(2-(3-(3-((4-((8-cyclopentyl-7-oxo- 7,8-dihydropyrido[2,3-d]pyrimidin-2- yl)amino)-piperidin-1- yl)sulfonyl)phenoxy)azetidin-1-yl)- ethoxy)-1-oxoisoindolin-2-yl)piperidine- 2,6-dione |
| 28 | 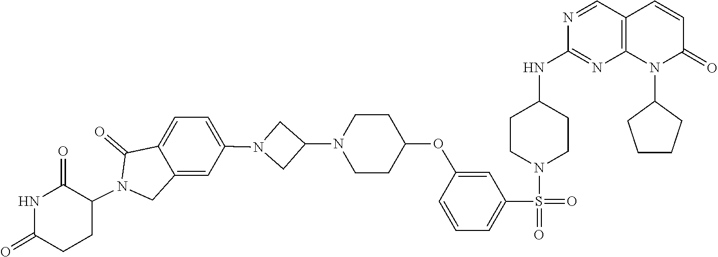 [SMILES: O=C1CCC(N2Cc3cc(N4CC(N5CCC(Oc6cccc(S(=O)(=O)N7CCC(Nc8ncc9ccc(=O)n(C%10CCCC%10)c9n8)CC7)c6)CC5)C4)ccc3C2=O)C(=O)N1] | 3-(5-(3-(4-(3-((4-((8-cyclopentyl-7-oxo- 7,8-dihydropyrido[2,3-d]pyrimidin-2- yl)amino)-piperidin-1- yl)sulfonyl)phenoxy)piperidin-1-yl)- azetidin-1-yl)-1-oxoisoindolin-2- yl)piperidine-2,6-dione |
| 29 | 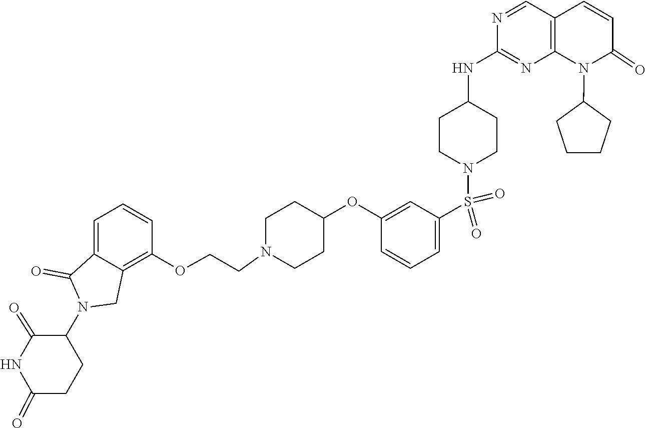 [SMILES: O=C1CCC(N2Cc3c(OCCN4CCC(Oc5cccc(S(=O)(=O)N6CCC(Nc7ncc8ccc(=O)n(C9CCCC9)c8n7)CC6)c5)CC4)cccc3C2=O)C(=O)N1] | 3-(4-(2-(4-(3-((4-((8-cyclopentyl-7-oxo- 7,8-dihydropyrido[2,3-d]pyrimidin-2- yl)amino)-piperidin-1- yl)sulfonyl)phenoxy)piperidin-1- yl)ethoxy)-1-oxoisoindolin-2- yl)piperidine-2,6-dione |
| 30 | 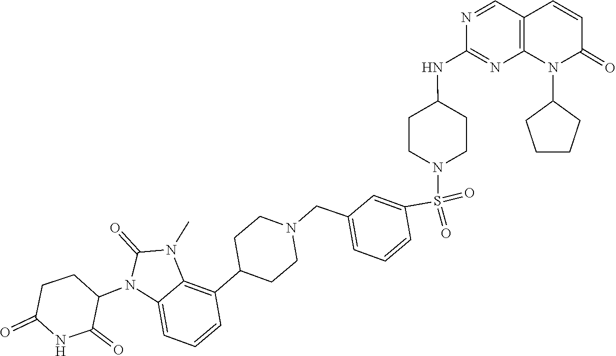 [SMILES: Cn1c(=O)n(C2CCC(=O)NC2=O)c2cccc(C3CCN(Cc4cccc(S(=O)(=O)N5CCC(Nc6ncc7ccc(=O)n(C8CCCC8)c7n6)CC5)c4)CC3)c21] | 3-(4-(1-(3-((4-((8-cyclopentyl-7-oxo-7,8- dihydro-pyrido[2,3-d]pyrimidin-2- yl)amino)piperidin-1- yl)sulfonyl)benzyl)piperidin-4-yl)-3- methyl-2-oxo-2,3-dihydro-1H- benzo[d]imidazol-1-yl)-piperidine-2,6- dione |
| 31 | 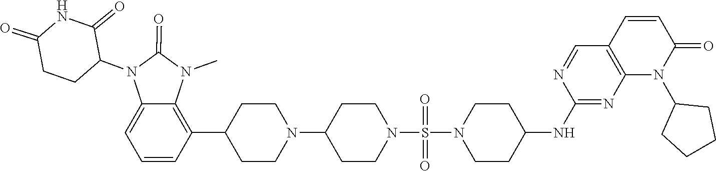 [SMILES: Cn1c(=O)n(C2CCC(=O)NC2=O)c2cccc(C3CCN(C4CCN(S(=O)(=O)N5CCC(Nc6ncc7ccc(=O)n(C8CCCC8)c7n6)CC5)CC4)CC3)c21] | 3-(4-(1′-((4-((8-cyclopentyl-7-oxo-7,8- dihydro-pyrido[2,3-d]pyrimidin-2- yl)amino)piperidin-1-yl)sulfonyl)-[1,4′- bipiperidin]-4-yl)-3-methyl-2-oxo-2,3- dihydro-1H-benzo[d]imidazol-1- yl)piperidine-2,6-dione |
| 32 | 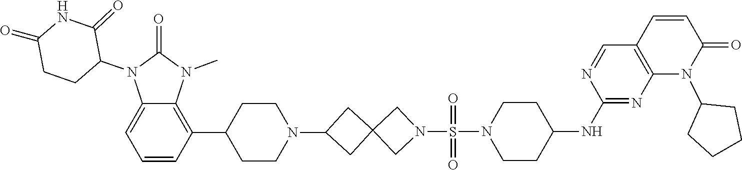 [SMILES: Cn1c(=O)n(C2CCC(=O)NC2=O)c2cccc(C3CCN(C4CC5(C4)CN(S(=O)(=O)N4CCC(Nc6ncc7ccc(=O)n(C8CCCC8)c7n6)CC4)C5)CC3)c21] | 3-(4-(1-(2-((4-((8-cyclopentyl-7-oxo-7,8- dihydro-pyrido[2,3-d]pyrimidin-2- yl)amino)piperidin-1-yl)sulfonyl)-2- azaspiro[3.3]heptan-6-yl)piperidin-4-yl)-3- methyl-2-oxo-2,3-dihydro-1H- benzo[d]imidazol-1-yl)piperidine-2,6- dione |
| 33 | 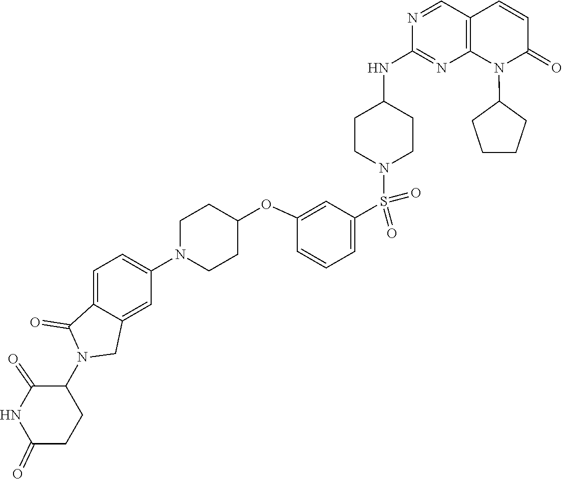 [SMILES: O=C1CCC(N2Cc3cc(N4CCC(Oc5cccc(S(=O)(=O)N6CCC(Nc7ncc8ccc(=O)n(C9CCCC9)c8n7)CC6)c5)CC4)ccc3C2=O)C(=O)N1] | 3-(5-(4-(3-((4-((8-cyclopentyl-7-oxo-7,8- dihydro-pyrido[2,3-d]pyrimidin-2- yl)amino)piperidin-1- yl)sulfonyl)phenoxy)piperidin-1-yl)-1- oxoiso-indolin-2-yl)piperidine-2,6-dione |
| 34 | 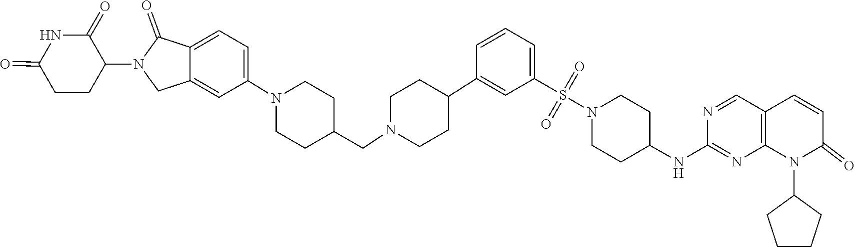 [SMILES: O=C1CCC(N2Cc3cc(N4CCC(CN5CCC(c6cccc(S(=O)(=O)N7CCC(Nc8ncc9ccc(=O)n(C%10CCCC%10)c9n8)CC7)c6)CC5)CC4)ccc3C2=O)C(=O)N1] | 3-(5-(4-((4-(3-((4-((8-cyclopentyl-7-oxo- 7,8-dihydropyrido[2,3-d]pyrimidin-2- yl)amino)-piperidin-1- yl)sulfonyl)phenyl)piperidin-1-yl)- methyl)piperidin-1-yl)-1-oxoisoindolin-2- yl)-piperidine-2,6-dione |
| 35 | 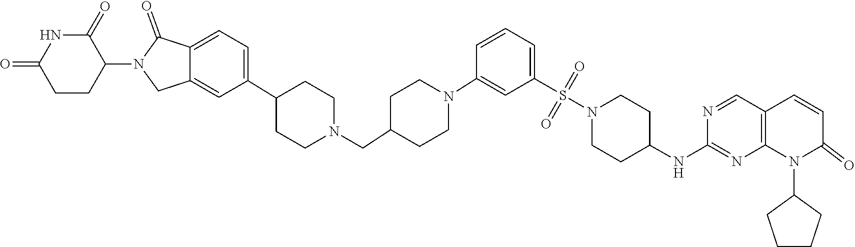 [SMILES: O=C1CCC(N2Cc3cc(C4CCN(CC5CCN(c6cccc(S(=O)(=O)N7CCC(Nc8ncc9ccc(=O)n(C%10CCCC%10)c9n8)CC7)c6)CC5)CC4)ccc3C2=O)C(=O)N1] | 3-(5-(1-((1-(3-((4-((8-cyclopentyl-7-oxo- 7,8-dihydropyrido[2,3-d]pyrimidin-2- yl)amino)-piperidin-1- yl)sulfonyl)phenyl)piperidin-4-yl)- methyl)piperidin-4-yl)-1-oxoisoindolin-2- yl)piperidine-2,6-dione |
| 36 | 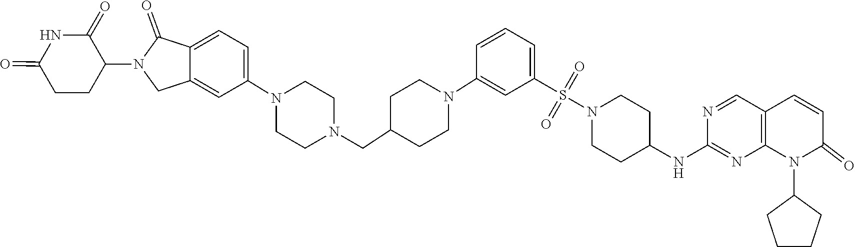 [SMILES: O=C1CCC(N2Cc3cc(N4CCN(CC5CCN(c6cccc(S(=O)(=O)N7CCC(Nc8ncc9ccc(=O)n(C%10CCCC%10)c9n8)CC7)c6)CC5)CC4)ccc3C2=O)C(=O)N1] | 3-(5-(4-((1-(3-((4-((8-cyclopentyl-7-oxo- 7,8-dihydropyrido[2,3-d]pyrimidin-2- yl)amino)-piperidin-1- yl)sulfonyl)phenyl)piperidin-4-yl)- methyl)piperazin-1-yl)-1-oxoisoindolin-2- yl)-piperidine-2,6-dione |
| 37 | 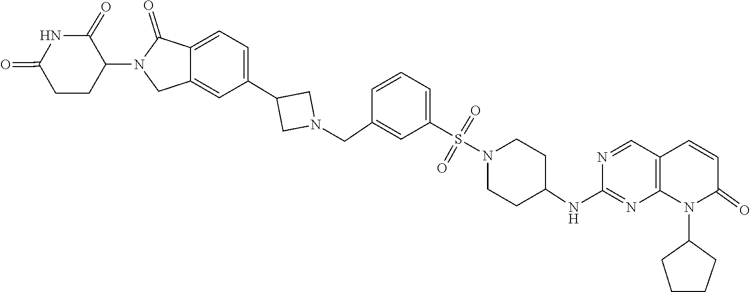 [SMILES: O=C1CCC(N2Cc3cc(C4CN(Cc5cccc(S(=O)(=O)N6CCC(Nc7ncc8ccc(=O)n(C9CCCC9)c8n7)CC6)c5)C4)ccc3C2=O)C(=O)N1] | 3-(5-(1-(3-((4-((8-cyclopentyl-7-oxo-7,8- dihydropyrido[2,3-d]pyrimidin-2- yl)amino)-piperidin-1- yl)sulfonyl)benzyl)azetidin-3-yl)-1- oxoisoindolin-2-yl)piperidine-2,6-dione |
| 38 | 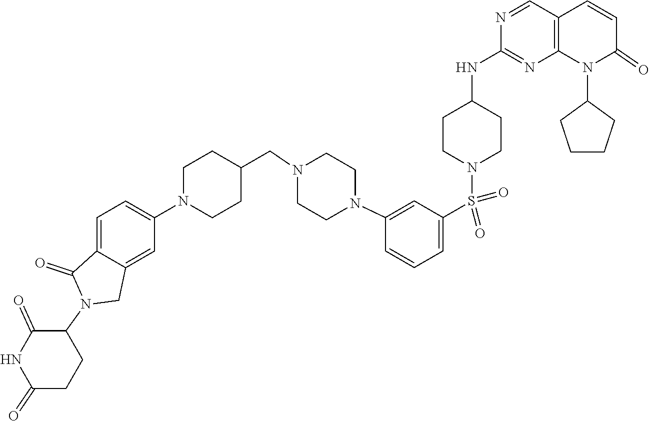 [SMILES: O=C1CCC(N2Cc3cc(N4CCC(CN5CCN(c6cccc(S(=O)(=O)N7CCC(Nc8ncc9ccc(=O)n(C%10CCCC%10)c9n8)CC7)c6)CC5)CC4)ccc3C2=O)C(=O)N1] | 3-(5-(4-((4-(3-((4-((8-cyclopentyl-7-oxo- 7,8-dihydropyrido[2,3-d]pyrimidin-2- yl)amino)-piperidin-1- yl)sulfonyl)phenyl)piperazin-1-yl)- methyl)piperidin-1-yl)-1-oxoisoindolin-2- yl)-piperidine-2,6-dione |
| 39 | 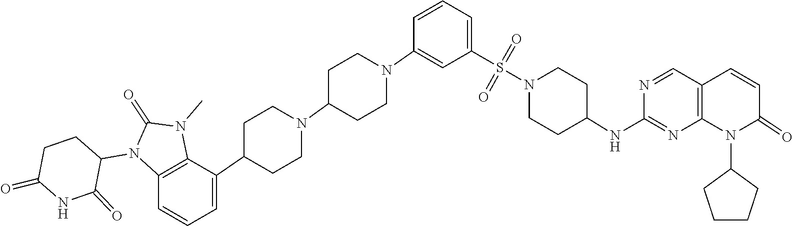 [SMILES: Cn1c(=O)n(C2CCC(=O)NC2=O)c2cccc(C3CCN(C4CCN(c5cccc(S(=O)(=O)N6CCC(Nc7ncc8ccc(=O)n(C9CCCC9)c8n7)CC6)c5)CC4)CC3)c21] | 3-(4-(1′-(3-((4-((8-cyclopentyl-7-oxo-7,8- dihydro-pyrido[2,3-d]pyrimidin-2- yl)amino)piperidin-1-yl)sulfonyl)phenyl)- [1,4′-bipiperidin]-4-yl)-3-methyl-2-oxo- 2,3-dihydro-1H-benzo[d]imidazol-1- yl)piperidine-2,6-dione |
| 40 | 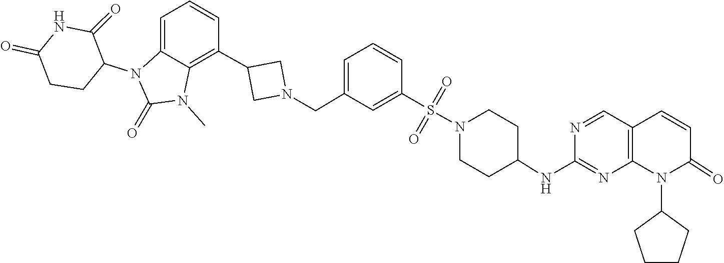 [SMILES: Cn1c(=O)n(C2CCC(=O)NC2=O)c2cccc(C3CN(Cc4cccc(S(=O)(=O)N5CCC(Nc6ncc7ccc(=O)n(C8CCCC8)c7n6)CC5)c4)C3)c21] | 3-(4-(1-(3-((4-((8-cyclopentyl-7-oxo-7,8- dihydropyrido[2,3-d]pyrimidin-2- yl)amino)piperidin-1- yl)sulfonyl)benzyl)azetidin-3-yl)-3- methyl-2-oxo-2,3-dihydro-1H- benzo[d]imidazol-1-yl)piperidine-2,6- dione |
| 41 | 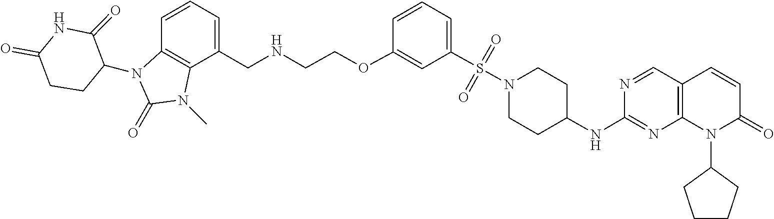 [SMILES: Cn1c(=O)n(C2CCC(=O)NC2=O)c2cccc(CNCCOc3cccc(S(=O)(=O)N4CCC(Nc5ncc6ccc(=O)n(C7CCCC7)c6n5)CC4)c3)c21] | 3-(4-(((2-(3-((4-((8-cyclopentyl-7-oxo-7,8- dihydropyrido[2,3-d]pyrimidin-2- yl)amino)-piperidin-1- yl)sulfonyl)phenoxy)ethyl)amino)- methyl)-3-methyl-2-oxo-2,3-dihydro-1H- benzo[d]imidazol-1-yl)piperidine-2,6- dione |
| 42 | 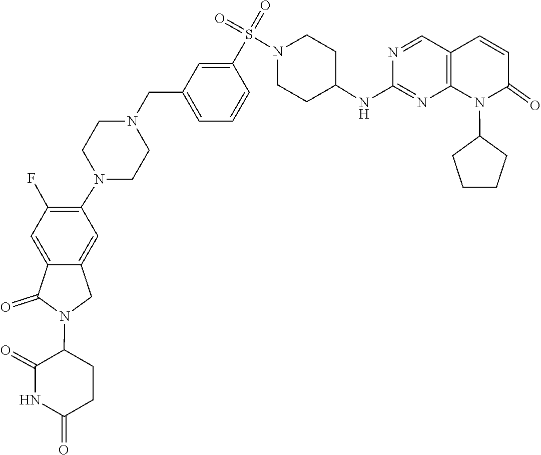 [SMILES: O=C1CCC(N2Cc3cc(N4CCN(Cc5cccc(S(=O)(=O)N6CCC(Nc7ncc8ccc(=O)n(C9CCCC9)c8n7)CC6)c5)CC4)c(F)cc3C2=O)C(=O)N1] | 3-(5-(4-(3-((4-((8-cyclopentyl-7-oxo-7,8- dihydropyrido[2,3-d]pyrimidin-2- yl)amino)-piperidin-1- yl)sulfonyl)benzyl)piperazin-1-yl)-6- fluoro-1-oxoisoindolin-2-yl)piperidine-2,6- dione |
| 43 | 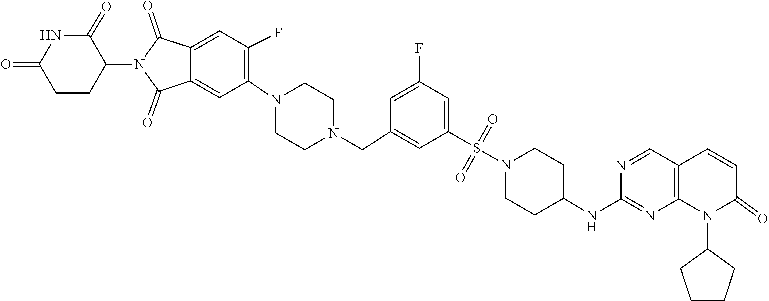 [SMILES: O=C1CCC(N2C(=O)c3cc(F)c(N4CCN(Cc5cc(F)cc(S(=O)(=O)N6CCC(Nc7ncc8ccc(=O)n(C9CCCC9)c8n7)CC6)c5)CC4)cc3C2=O)C(=O)N1] | 5-(4-(3-((4-((8-cyclopentyl-7-oxo-7,8- dihydro-pyrido[2,3-d]pyrimidin-2- yl)amino)piperidin-1-yl)sulfonyl)-5- fluorobenzyl)piperazin-1-yl)-2-(2,6- dioxopiperidin-3-yl)-6-fluoroisoindoline- 1,3-dione |
| 44 | 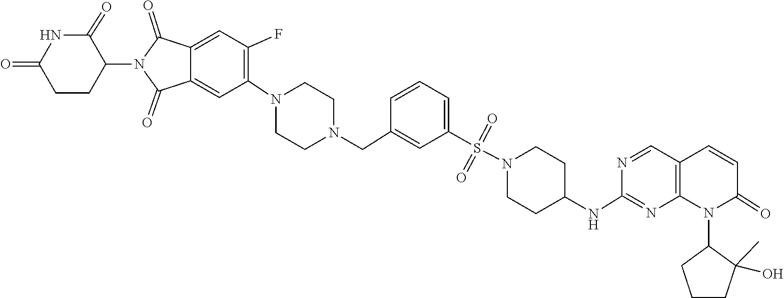 [SMILES: CC1(O)CCCC1n1c(=O)ccc2cnc(NC3CCN(S(=O)(=O)c4cccc(CN5CCN(c6cc7c(cc6F)C(=O)N(C6CCC(=O)NC6=O)C7=O)CC5)c4)CC3)nc21] | 2-(2,6-dioxopiperidin-3-yl)-5-fluoro-6-(4- (3-((4-((8-(2-hydroxy-2- methylcyclopentyl)-7-oxo-7,8- dihydropyrido[2,3-d]pyrimidin-2- yl)amino)-piperidin-1- yl)sulfonyl)benzyl)piperazin-1-yl)- isoindoline-1,3-dione |
| 45 | 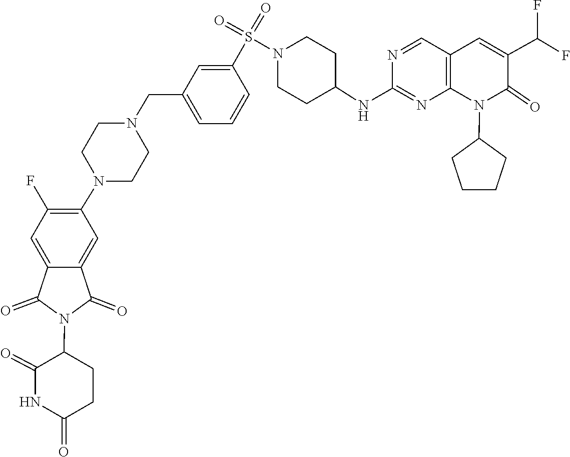 [SMILES: O=C1CCC(N2C(=O)c3cc(F)c(N4CCN(Cc5cccc(S(=O)(=O)N6CCC(Nc7ncc8cc(C(F)F)c(=O)n(C9CCCC9)c8n7)CC6)c5)CC4)cc3C2=O)C(=O)N1] | 5-(4-(3-((4-((8-cyclopentyl-6- (difluoromethyl)-7-oxo-7,8- dihydropyrido[2,3-d]pyrimidin-2-yl)- amino)piperidin-1-yl)sulfonyl)- benzyl)piperazin-1-yl)-2-(2,6- dioxopiperidin-3-yl)-6-fluoro-isoindoline- 1,3-dione |
| 46 | 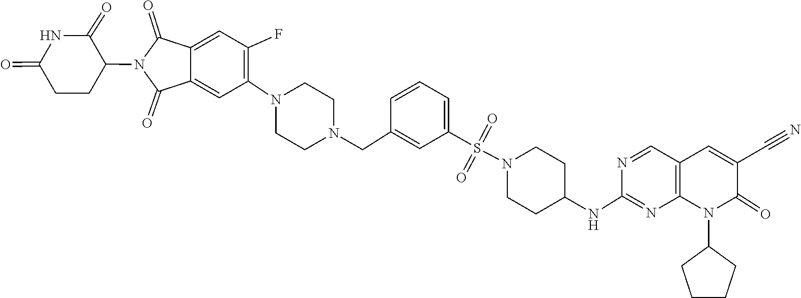 [SMILES: N#Cc1cc2cnc(NC3CCN(S(=O)(=O)c4cccc(CN5CCN(c6cc7c(cc6F)C(=O)N(C6CCC(=O)NC6=O)C7=O)CC5)c4)CC3)nc2n(C2CCCC2)c1=O] | 8-cyclopentyl-2-((1-((3-((4-(2-(2,6- dioxopiperidin-3-yl)-6-fluoro-1,3- dioxoisoindolin-5-yl)piperazin-1- yl)methyl)phenyl)sulfonyl)piperidin-4-yl)- amino)-7-oxo-7,8-dihydropyrido[2,3-d]- pyrimidine-6-carbonitrile |
| 47 | 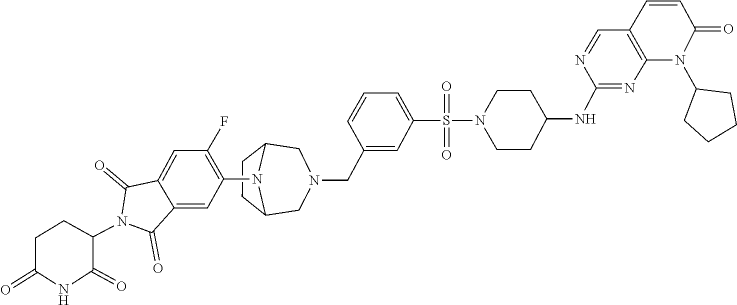 [SMILES: O=C1CCC(N2C(=O)c3cc(F)c(N4C5CCC4CN(Cc4cccc(S(=O)(=O)N6CCC(Nc7ncc8ccc(=O)n(C9CCCC9)c8n7)CC6)c4)C5)cc3C2=O)C(=O)N1] | 5-(3-(3-((4-((8-cyclopentyl-7-oxo-7,8- dihydro-pyrido[2,3-d]pyrimidin-2- yl)amino)piperidin-1-yl)sulfonyl)benzyl)- 3,8-diazabicyclo[3.2.1]octan-8-yl)-2-(2,6- dioxopiperidin-3-yl)-6-fluoro-isoindoline- 1,3-dione |
| 48 | 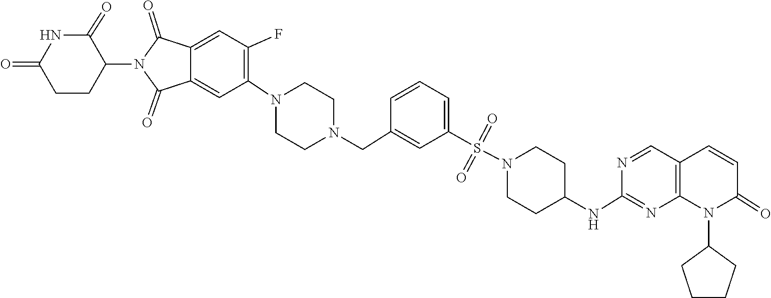 [SMILES: O=C1CCC(N2C(=O)c3cc(F)c(N4CCN(Cc5cccc(S(=O)(=O)N6CCC(Nc7ncc8ccc(=O)n(C9CCCC9)c8n7)CC6)c5)CC4)cc3C2=O)C(=O)N1] | 5-(4-(3-((4-((8-cyclopentyl-7-oxo-7,8- dihydro-pyrido[2,3-d]pyrimidin-2- yl)amino)piperidin-1-yl)sulfonyl)- benzyl)piperazin-1-yl)-2-(2,6-dioxo- piperidin-3-yl)-6-fluoroisoindoline-1,3- dione |
| 49 | 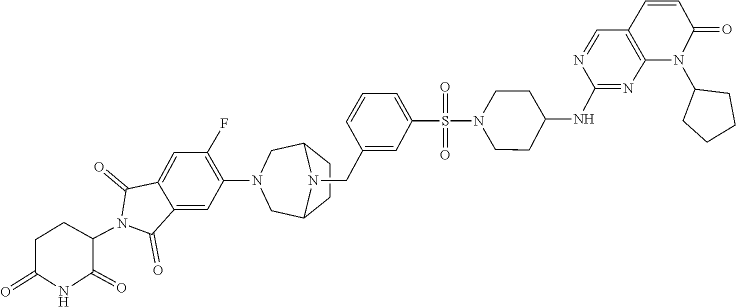 [SMILES: O=C1CCC(N2C(=O)c3cc(F)c(N4CC5CCC(C4)N5Cc4cccc(S(=O)(=O)N5CCC(Nc6ncc7ccc(=O)n(C8CCCC8)c7n6)CC5)c4)cc3C2=O)C(=O)N1] | 5-(8-(3-((4-((8-cyclopentyl-7-oxo-7,8- dihydro-pyrido[2,3-d]pyrimidin-2- yl)amino)piperidin-1-yl)sulfonyl)benzyl)- 3,8-diazabicyclo[3.2.1]octan-3-yl)-2-(2,6- dioxopiperidin-3-yl)-6-fluoroi-soindoline- 1,3-dione |
| 50 | 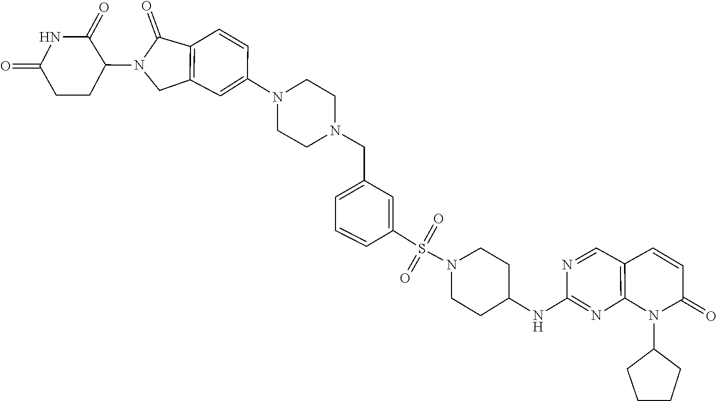 [SMILES: O=C1CCC(N2Cc3cc(N4CCN(Cc5cccc(S(=O)(=O)N6CCC(Nc7ncc8ccc(=O)n(C9CCCC9)c8n7)CC6)c5)CC4)ccc3C2=O)C(=O)N1] | 3-(5-(4-(3-((4-((8-cyclopentyl-7-oxo-7,8- dihydropyrido[2,3-d]pyrimidin-2-yl)- amino)-piperidin-1-yl)sulfonyl)benzyl)- piperazin-1-yl)-1-oxoisoindolin-2- yl)piperidine-2,6-dione |
| 51 | 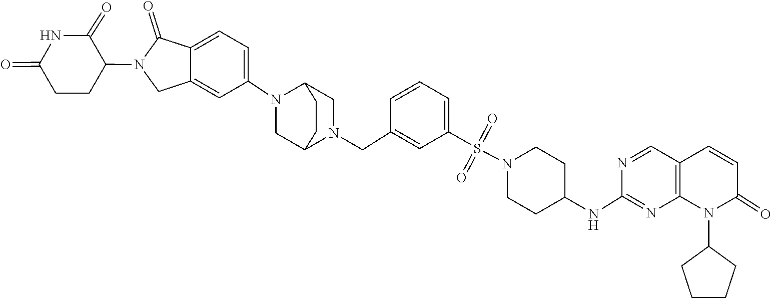 [SMILES: O=C1CCC(N2Cc3cc(N4CC5CCC4CN5Cc4cccc(S(=O)(=O)N5CCC(Nc6ncc7ccc(=O)n(C8CCCC8)c7n6)CC5)c4)ccc3C2=O)C(=O)N1] | 3-(5-(5-(3-((4-((8-cyclopentyl-7-oxo-7,8- dihydropyrido[2,3-d]pyrimidin-2- yl)amino)-piperidin-1-yl)sulfonyl)benzyl)- 2,5-diazabicyclo[2.2.2]octan- 2-yl)-1-oxoisoindolin-2-yl)piperidine-2,6- dione |
| 52 | 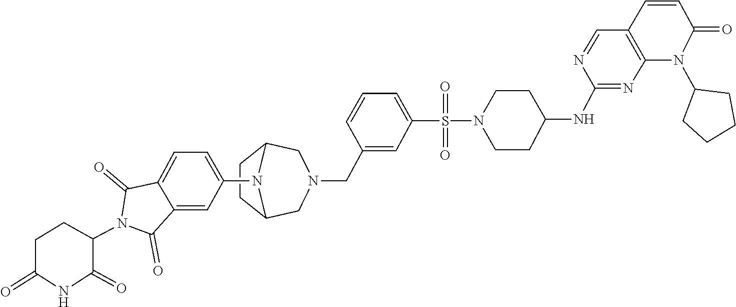 [SMILES: O=C1CCC(N2C(=O)c3ccc(N4C5CCC4CN(Cc4cccc(S(=O)(=O)N6CCC(Nc7ncc8ccc(=O)n(C9CCCC9)c8n7)CC6)c4)C5)cc3C2=O)C(=O)N1] | 3-(5-(3-(3-((4-((8-cyclopentyl-7-oxo-7,8- dihydropyrido[2,3-d]pyrimidin-2- yl)amino)-piperidin-1-yl)sulfonyl)benzyl)- 3,8-diazabicyclo-[3.2.1]octan-8-yl)-1- oxoisoindolin-2-yl)piperidine-2,6-dione |
| 53 | 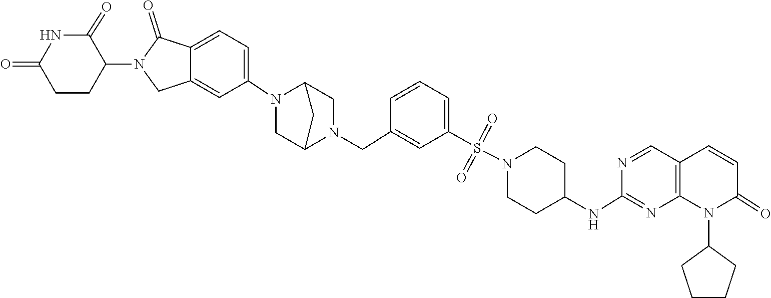 [SMILES: O=C1CCC(N2Cc3cc(N4CC5CC4CN5Cc4cccc(S(=O)(=O)N5CCC(Nc6ncc7ccc(=O)n(C8CCCC8)c7n6)CC5)c4)ccc3C2=O)C(=O)N1] | 3-(5-(5-(3-((4-((8-cyclopentyl-7-oxo-7,8- dihydropyrido[2,3-d]pyrimidin-2- yl)amino)-piperidin-1-yl)sulfonyl)benzyl)- 2,5-diazabicyclo-[2.2.1]heptan-2-yl)-1- oxoisoindolin-2-yl)-piperidine-2,6-dione |
| 54 | 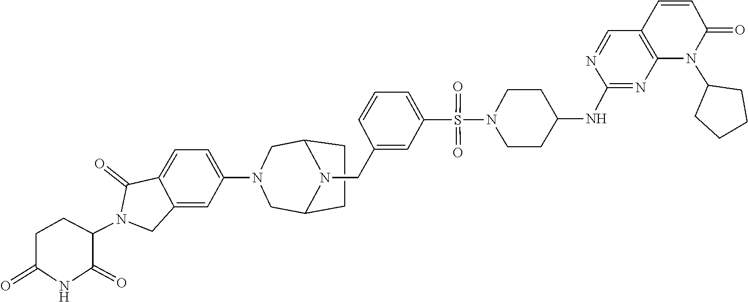 [SMILES: O=C1CCC(N2Cc3cc(N4CC5CCC(C4)N5Cc4cccc(S(=O)(=O)N5CCC(Nc6ncc7ccc(=O)n(C8CCCC8)c7n6)CC5)c4)ccc3C2=O)C(=O)N1] | 3-(5-(8-(3-((4-((8-cyclopentyl-7-oxo-7,8- dihydro-pyrido[2,3-d]pyrimidin-2- yl)amino)piperidin-1-yl)sulfonyl)benzyl)- 3,8-diazabicyclo[3.2.1]octan-3-yl)-1- oxoisoindolin-2-yl)piperidine-2,6-dione |
| 55 | 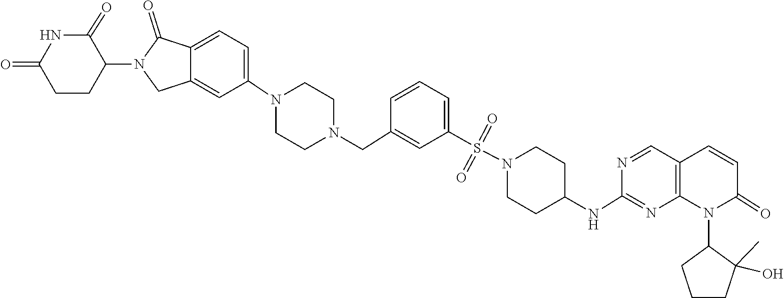 [SMILES: CC1(O)CCCC1n1c(=O)ccc2cnc(NC3CCN(S(=O)(=O)c4cccc(CN5CCN(c6ccc7c(c6)CN(C6CCC(=O)NC6=O)C7=O)CC5)c4)CC3)nc21] | 3-(5-(4-(3-((4-((8-(2-hydroxy-2-methyl- cyclopentyl)-7-oxo-7,8-dihydropyrido[2,3- d]-pyrimidin-2-yl)amino)piperidin-1- yl)sulfonyl)-benzyl)piperazin-1-yl)-1- oxoisoindolin-2-yl)-piperidine-2,6-dione |
| 56 | 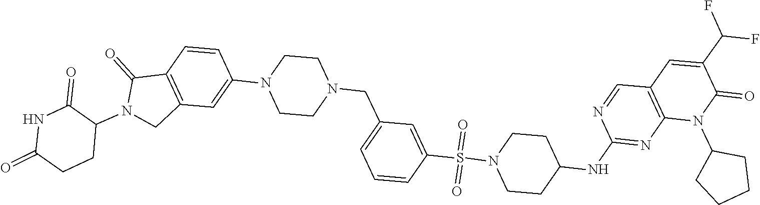 [SMILES: O=C1CCC(N2Cc3cc(N4CCN(Cc5cccc(S(=O)(=O)N6CCC(Nc7ncc8cc(C(F)F)c(=O)n(C9CCCC9)c8n7)CC6)c5)CC4)ccc3C2=O)C(=O)N1] | 3-(5-(4-(3-((4-((8-cyclopentyl-6- (difluoromethyl)-7-oxo-7,8- dihydropyrido[2,3-d]pyrimidin-2- yl)amino)piperidin-1- yl)sulfonyl)benzyl)piperazin-1-yl)-1- oxoisoindolin-2-yl)piperidine-2,6-dione |
| 57 | 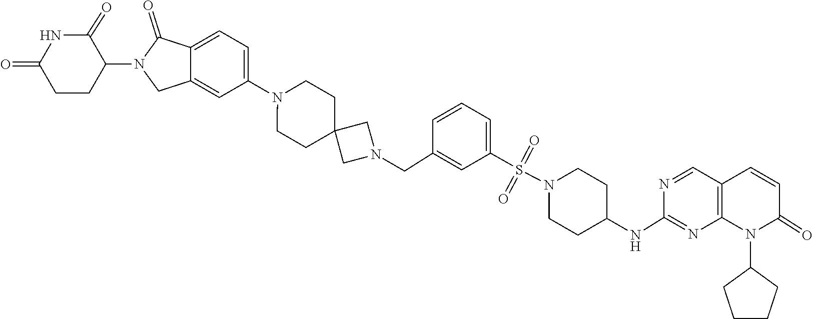 [SMILES: O=C1CCC(N2Cc3cc(N4CCC5(CC4)CN(Cc4cccc(S(=O)(=O)N6CCC(Nc7ncc8ccc(=O)n(C9CCCC9)c8n7)CC6)c4)C5)ccc3C2=O)C(=O)N1] | 3-(5-(2-(3-((4-((8-cyclopentyl-7-oxo-7,8- dihydro-pyrido[2,3-d]pyrimidin-2- yl)amino)piperidin-1-yl)sulfonyl)benzyl)- 2,7-diazaspiro[3.5]nonan-7-yl)-1- oxoisoindolin-2-yl)piperidine-2,6-dione |
| 58 | 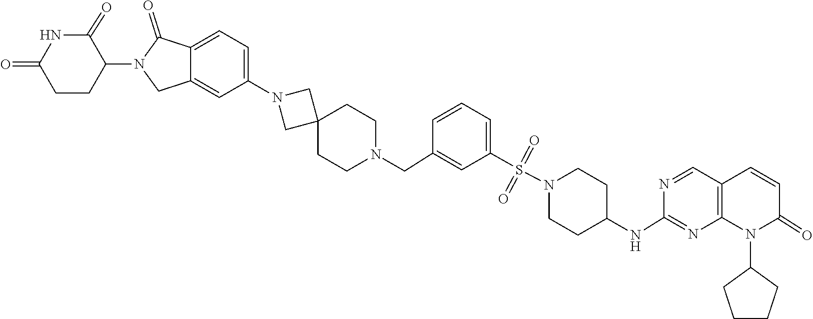 [SMILES: O=C1CCC(N2Cc3cc(N4CC5(CCN(Cc6cccc(S(=O)(=O)N7CCC(Nc8ncc9ccc(=O)n(C%10CCCC%10)c9n8)CC7)c6)CC5)C4)ccc3C2=O)C(=O)N1] | 3-(5-(7-(3-((4-((8-cyclopentyl-7-oxo-7,8- dihydropyrido[2,3-d]pyrimidin-2- yl)amino)-piperidin-1-yl)sulfonyl)benzyl)- 2,7-diazaspiro-[3.5]nonan-2-yl)-1- oxoisoindolin-2-yl)piperidine-2,6-dione |
| 59 | 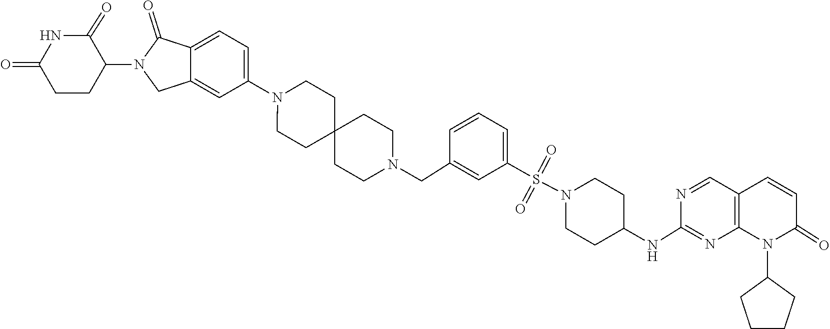 [SMILES: O=C1CCC(N2Cc3cc(N4CCC5(CCN(Cc6cccc(S(=O)(=O)N7CCC(Nc8ncc9ccc(=O)n(C%10CCCC%10)c9n8)CC7)c6)CC5)CC4)ccc3C2=O)C(=O)N1] | 3-(5-(9-(3-((4-((8-cyclopentyl-7-oxo-7,8- dihydro-pyrido[2,3-d]pyrimidin-2- yl)amino)piperidin-1-yl)sulfonyl)benzyl)- 3,9-diazaspiro[5.5]undecan-3-yl)-1- oxoisoindolin-2-yl)piperidine-2,6-dione |
| 60 | 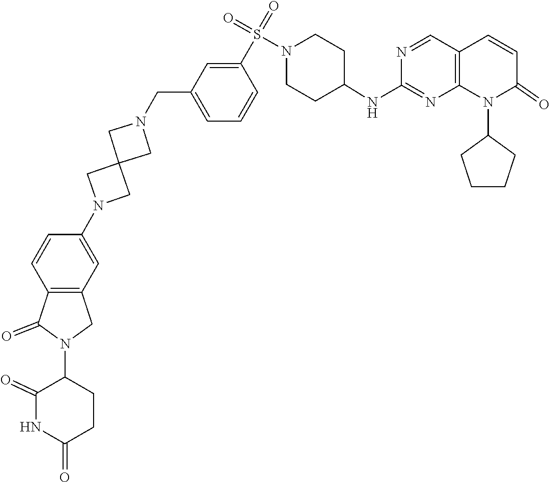 [SMILES: O=C1CCC(N2Cc3cc(N4CC5(CN(Cc6cccc(S(=O)(=O)N7CCC(Nc8ncc9ccc(=O)n(C%10CCCC%10)c9n8)CC7)c6)C5)C4)ccc3C2=O)C(=O)N1] | 3-(5-(6-(3-((4-((8-cyclopentyl-7-oxo-7,8- dihydro-pyrido[2,3-d]pyrimidin-2- yl)amino)piperidin-1-yl)sulfonyl)benzyl)- 2,6-diazaspiro[3.3]heptan-2-yl)-1- oxoisoindolin-2-yl)piperidine-2,6-dione |
| 61 | 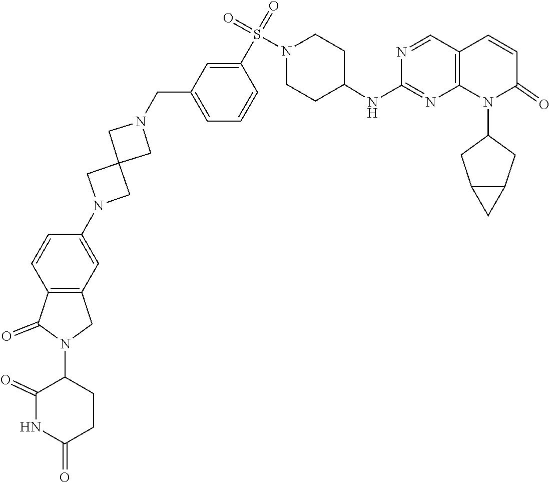 [SMILES: O=C1CCC(N2Cc3cc(N4CC5(CN(Cc6cccc(S(=O)(=O)N7CCC(Nc8ncc9ccc(=O)n(C%10CC%11CC%11C%10)c9n8)CC7)c6)C5)C4)ccc3C2=O)C(=O)N1] | 3-(5-(6-(3-((4-((8-(bicyclo[3.1.0]hexan-3- yl)-7-oxo-7,8-dihydropyrido[2,3- d]pyrimidin-2-yl)amino)piperidin-1- yl)sulfonyl)benzyl)-2,6- diazaspiro[3.3]heptan-2-yl)-1- oxoisoindolin-2-yl)piperidine-2,6-dione |
| 62 | 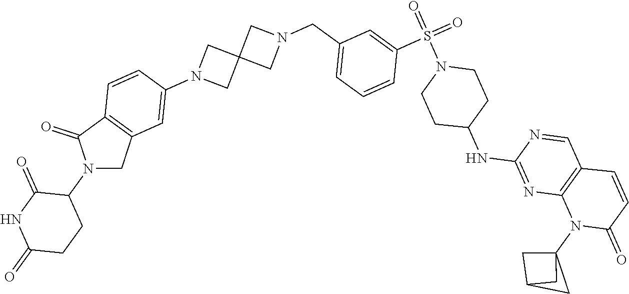 [SMILES: O=C1CCC(N2Cc3cc(N4CC5(CN(Cc6cccc(S(=O)(=O)N7CCC(Nc8ncc9ccc(=O)n(C%10%11CC(C%10)C%11)c9n8)CC7)c6)C5)C4)ccc3C2=O)C(=O)N1] | 3-(5-(6-(3-((4-((8-(bicyclo[1.1.1]pentan-1- yl)-7-oxo-7,8-dihydropyrido[2,3- d]pyrimidin-2-yl)-amino)piperidin-1- yl)sulfonyl)benzyl)-2,6- diazaspiro[3.3]heptan-2-yl)-1- oxoisoindolin-2-yl)-piperidine-2,6-dione |
| 63 | 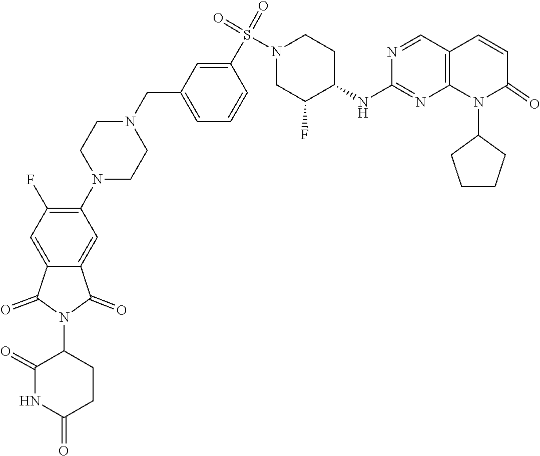 [SMILES: O=C1CCC(N2C(=O)c3cc(F)c(N4CCN(Cc5cccc(S(=O)(=O)N6CC[C@H](Nc7ncc8ccc(=O)n(C9CCCC9)c8n7)[C@H](F)C6)c5)CC4)cc3C2=O)C(=O)N1] | 5-(4-(3-(((3R,4S)-4-((8-cyclopentyl-7-oxo- 7,8-dihydropyrido[2,3-d]pyrimidin-2- yl)amino)-3-fluoropiperidin-1- yl)sulfonyl)benzyl)piperazin-1-yl)-2-(2,6- dioxopiperidin-3-yl)-6-fluoroiso-indoline- 1,3-dione |
| 64 | 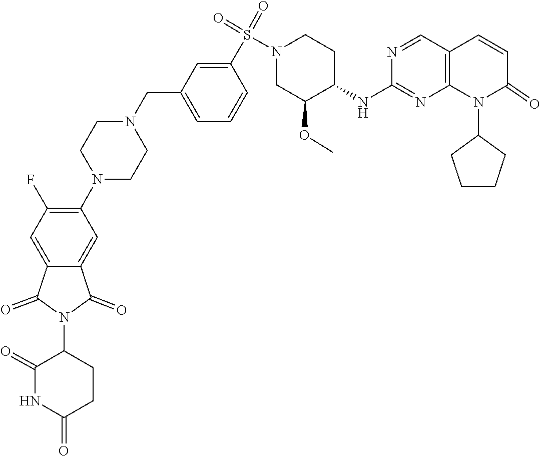 [SMILES: CO[C@H]1CN(S(=O)(=O)c2cccc(CN3CCN(c4cc5c(cc4F)C(=O)N(C4CCC(=O)NC4=O)C5=O)CC3)c2)CC[C@@H]1Nc1ncc2ccc(=O)n(C3CCCC3)c2n1] | 5-(4-(3-(((3S,4S)-4-((8-cyclopentyl-7-oxo- 7,8-dihydropyrido[2,3-d]pyrimidin-2- yl)amino)-3-methoxypiperidin-1- yl)sulfonyl)benzyl)piperazin-1-yl)-2-(2,6- dioxopiperidin-3-yl)-6-fluoro-isoindoline- 1,3-dione |
| 65 | 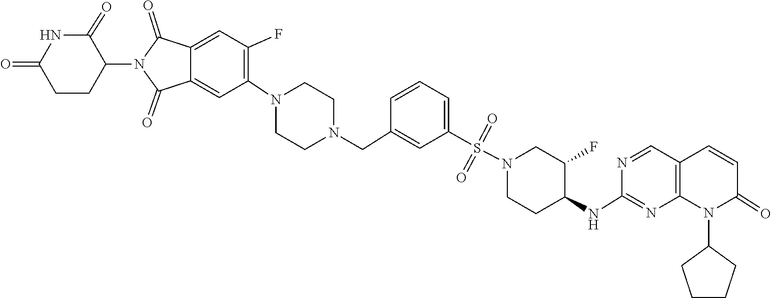 [SMILES: O=C1CCC(N2C(=O)c3cc(F)c(N4CCN(Cc5cccc(S(=O)(=O)N6CC[C@H](Nc7ncc8ccc(=O)n(C9CCCC9)c8n7)[C@@H](F)C6)c5)CC4)cc3C2=O)C(=O)N1] | 5-(4-(3-(((3S,4S)-4-((8-cyclopentyl-7-oxo- 7,8-dihydropyrido[2,3-d]pyrimidin-2- yl)amino)-3-fluoropiperidin-1- yl)sulfonyl)benzyl)piperazin-1-yl)-2-(2,6- dioxopiperidin-3-yl)-6-fluoro-isoindoline- 1,3-dione |
| 66 | 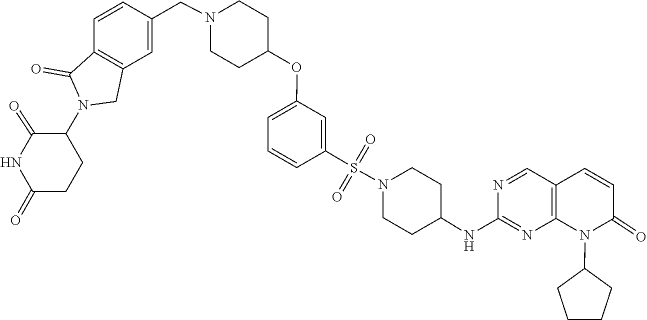 [SMILES: O=C1CCC(N2Cc3cc(CN4CCC(Oc5cccc(S(=O)(=O)N6CCC(Nc7ncc8ccc(=O)n(C9CCCC9)c8n7)CC6)c5)CC4)ccc3C2=O)C(=O)N1] | 3-(5-((4-(3-((4-((8-cyclopentyl-7-oxo-7,8- dihydropyrido[2,3-d]pyrimidin-2- yl)amino)-piperidin-1- yl)sulfonyl)phenoxy)-piperidin-1-yl)- methyl)-1-oxoisoindolin-2-yl)piperidine- 2,6-dione |
| 67 | 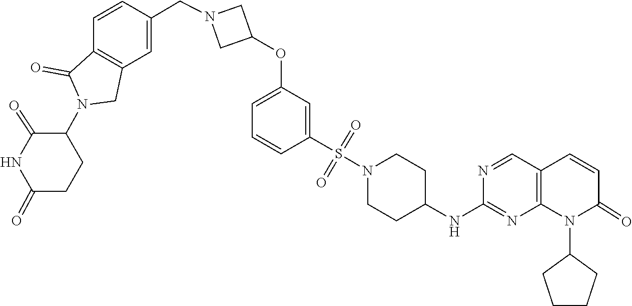 [SMILES: O=C1CCC(N2Cc3cc(CN4CC(Oc5cccc(S(=O)(=O)N6CCC(Nc7ncc8ccc(=O)n(C9CCCC9)c8n7)CC6)c5)C4)ccc3C2=O)C(=O)N1] | 3-(5-((3-(3-((4-((8-cyclopentyl-7-oxo-7,8- dihydro-pyrido[2,3-d]pyrimidin-2- yl)amino)piperidin-1- yl)sulfonyl)phenoxy)azetidin-1- yl)methyl)-1-oxoisoindolin-2- yl)piperidine-2,6-dione |
| 68 | 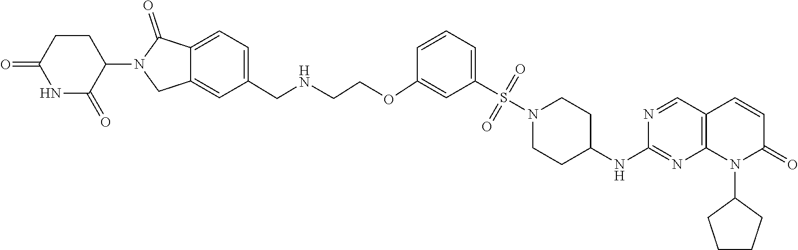 [SMILES: O=C1CCC(N2Cc3cc(CNCCOc4cccc(S(=O)(=O)N5CCC(Nc6ncc7ccc(=O)n(C8CCCC8)c7n6)CC5)c4)ccc3C2=O)C(=O)N1] | 3-(5-(((2-(3-((4-((8-cyclopentyl-7-oxo-7,8- dihydropyrido[2,3-d]pyrimidin-2- yl)amino)-piperidin-1- yl)sulfonyl)phenoxy)ethyl)amino)- methyl)-1-oxoisoindolin-2-yl)piperidine- 2,6-dione |
| 69 | 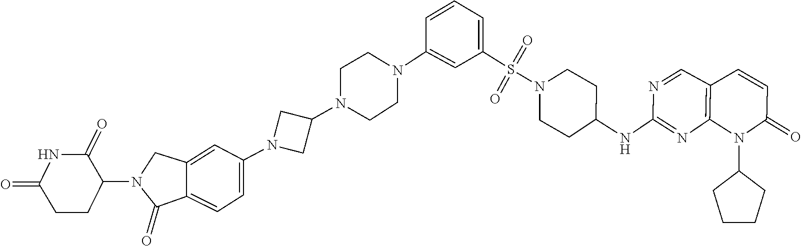 [SMILES: O=C1CCC(N2Cc3cc(N4CC(N5CCN(c6cccc(S(=O)(=O)N7CCC(Nc8ncc9ccc(=O)n(C%10CCCC%10)c9n8)CC7)c6)CC5)C4)ccc3C2=O)C(=O)N1] | 3-(5-(3-(4-(3-((4-((8-cyclopentyl-7-oxo- 7,8-dihydropyrido[2,3-d]pyrimidin-2- yl)amino)-piperidin-1- yl)sulfonyl)phenyl)piperazin-1-yl)azetidin- 1-yl)-1-oxoisoindolin-2-yl)piperidine-2,6- dione |
| 70 | 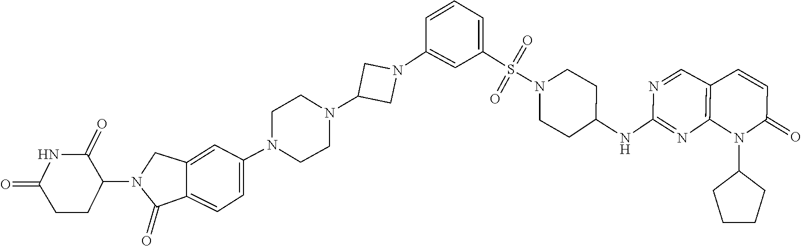 [SMILES: O=C1CCC(N2Cc3cc(N4CCN(C5CN(c6cccc(S(=O)(=O)N7CCC(Nc8ncc9ccc(=O)n(C%10CCCC%10)c9n8)CC7)c6)C5)CC4)ccc3C2=O)C(=O)N1] | 3-(5-(4-(1-(3-((4-((8-cyclopentyl-7-oxo- 7,8-dihydropyrido[2,3-d]pyrimidin-2- yl)amino)-piperidin-1-yl)sulfonyl)phenyl)- azetidin-3-yl)piperazin-1-yl)-1- oxoisoindolin-2-yl)piperidine-2,6-dione |
| 71 | 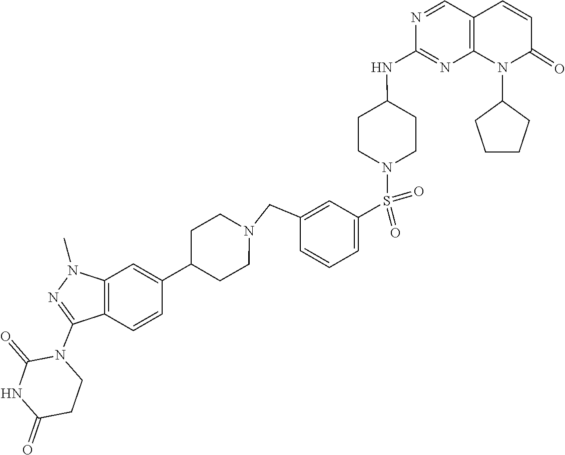 [SMILES: Cn1nc(N2CCC(=O)NC2=O)c2ccc(C3CCN(Cc4cccc(S(=O)(=O)N5CCC(Nc6ncc7ccc(=O)n(C8CCCC8)c7n6)CC5)c4)CC3)cc21] | 1-(6-(1-(3-((4-((8-cyclopentyl-7-oxo-7,8- dihydropyrido[2,3-d]pyrimidin-2- yl)amino)-piperidin-1-yl)sulfonyl)- benzyl)piperidin-4-yl)-1-methyl-1H- indazol-3-yl)dihydropyrimidine- 2,4(1H,3H)-dione |
| 72 | 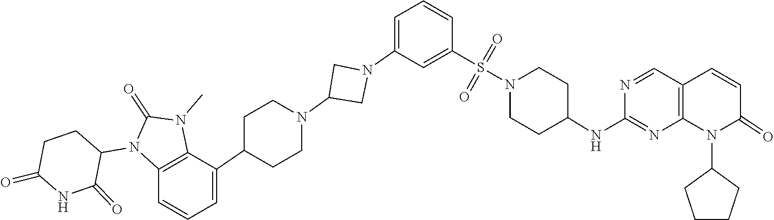 [SMILES: Cn1c(=O)n(C2CCC(=O)NC2=O)c2cccc(C3CCN(C4CN(c5cccc(S(=O)(=O)N6CCC(Nc7ncc8ccc(=O)n(C9CCCC9)c8n7)CC6)c5)C4)CC3)c21] | 3-(4-(1-(1-(3-((4-((8-cyclopentyl-7-oxo- 7,8-dihydropyrido[2,3-d]pyrimidin-2- yl)amino)-piperidin-1- yl)sulfonyl)phenyl)azetidin-3-yl)piperidin- 4-yl)-3-methyl-2-oxo-2,3-dihydro-1H- benzo[d]imidazol-1-yl)piperidine-2,6- dione |
| 73 | 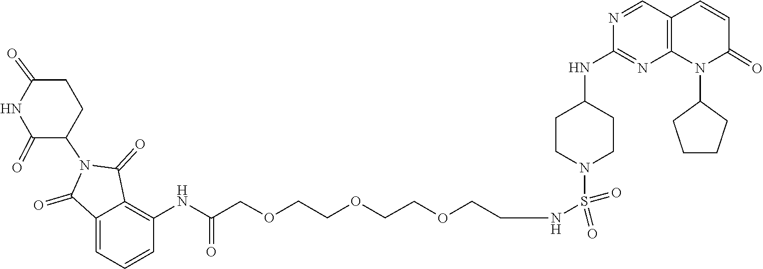 [SMILES: O=C1CCC(N2C(=O)c3cccc(NC(=O)COCCOCCOCCNS(=O)(=O)N4CCC(Nc5ncc6ccc(=O)n(C7CCCC7)c6n5)CC4)c3C2=O)C(=O)N1] | 2-(2-(2-(2-((4-((8-cyclopentyl-7-oxo-7,8- dihydro-pyrido[2,3-d]pyrimidin-2- yl)amino)piperidine)-1- sulfonamido)ethoxy)ethoxy)ethoxy)-N-(2- (2,6-dioxopiperidin-3-yl)-1,3- dioxoisoindolin-4-yl)-acetamide |
| 74 | 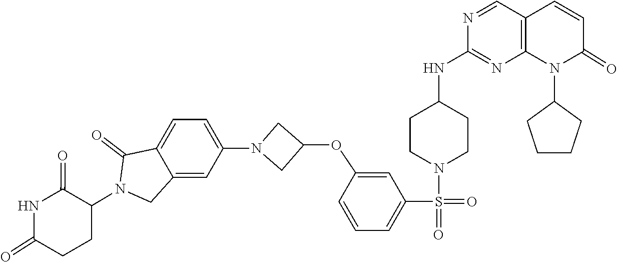 [SMILES: O=C1CCC(N2Cc3cc(N4CC(Oc5cccc(S(=O)(=O)N6CCC(Nc7ncc8ccc(=O)n(C9CCCC9)c8n7)CC6)c5)C4)ccc3C2=O)C(=O)N1] | 3-(5-(3-(3-((4-((8-cyclopentyl-7-oxo-7,8- dihydro-pyrido[2,3-d]pyrimidin-2- yl)amino)piperidin-1- yl)sulfonyl)phenoxy)azetidin-1-yl)-1- oxoisoindolin-2-yl)piperidine-2,6-dione |
| 75 | 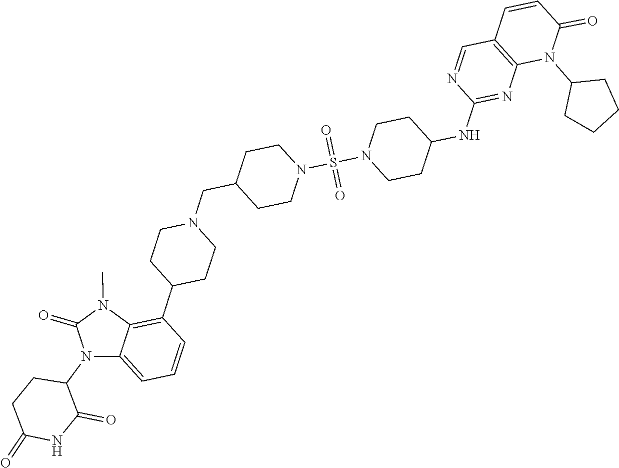 [SMILES: Cn1c(=O)n(C2CCC(=O)NC2=O)c2cccc(C3CCN(CC4CCN(S(=O)(=O)N5CCC(Nc6ncc7ccc(=O)n(C8CCCC8)c7n6)CC5)CC4)CC3)c21] | 3-(4-(1-((1-((4-((8-cyclopentyl-7-oxo-7,8- dihydropyrido[2,3-d]pyrimidin-2- yl)amino)-piperidin-1-yl)sulfonyl)- piperidin-4-yl)methyl)-piperidin-4-yl)-3- methyl-2-oxo-2,3-dihydro-1H- benzo[d]imidazol-1-yl)piperidine-2,6- dione |
| 76 | 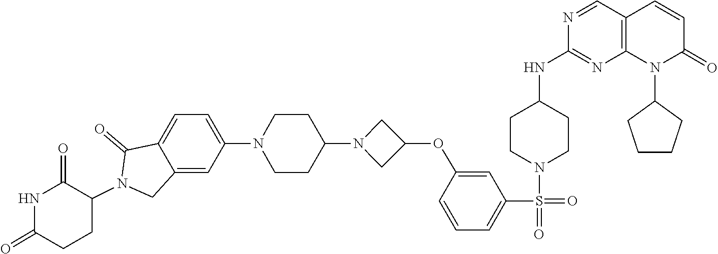 [SMILES: O=C1CCC(N2Cc3cc(N4CCC(N5CC(Oc6cccc(S(=O)(=O)N7CCC(Nc8ncc9ccc(=O)n(C%10CCCC%10)c9n8)CC7)c6)C5)CC4)ccc3C2=O)C(=O)N1] | 3-(5-(4-(3-(3-((4-((8-cyclopentyl-7-oxo- 7,8-dihydropyrido[2,3-d]pyrimidin-2- yl)amino)-piperidin-1- yl)sulfonyl)phenoxy)azetidin-1- yl)piperidin-1-yl)-1-oxoisoindolin-2- yl)piperidine-2,6-dione |

Contemplated compounds of Formula (IA′) are provided in Compound Table II below:

Compound Table II

| Cpd. | |
|---|---|
| No. | Structure |
| II-1 | 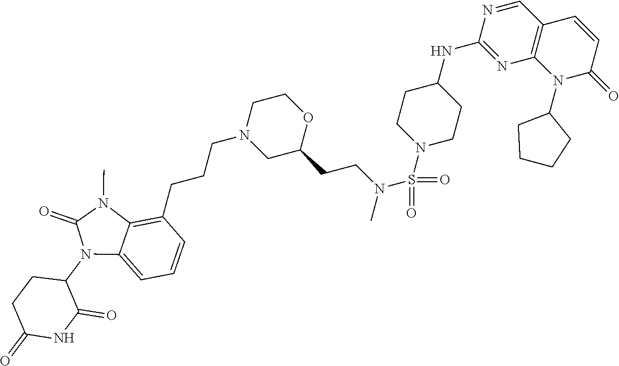 [SMILES: CN(CC[C@H]1CN(CCCc2cccc3c2n(C)c(=O)n3C2CCC(=O)NC2=O)CCO1)S(=O)(=O)N1CCC(Nc2ncc3ccc(=O)n(C4CCCC4)c3n2)CC1] |
| II-2 | 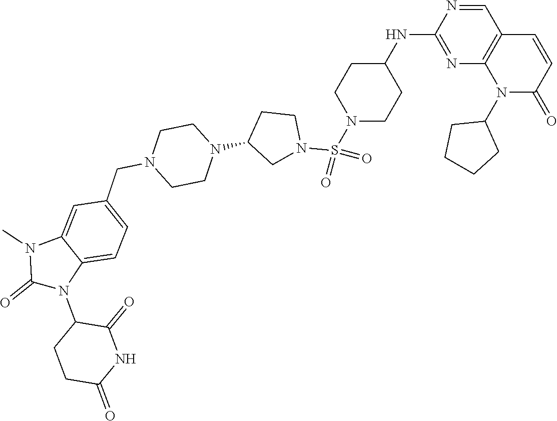 [SMILES: Cn1c(=O)n(C2CCC(=O)NC2=O)c2ccc(CN3CCN([C@@H]4CCN(S(=O)(=O)N5CCC(Nc6ncc7ccc(=O)n(C8CCCC8)c7n6)CC5)C4)CC3)cc21] |
| II-3 | 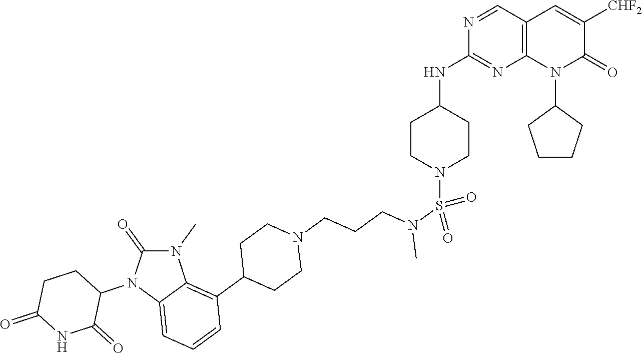 [SMILES: CN(CCCN1CCC(c2cccc3c2n(C)c(=O)n3C2CCC(=O)NC2=O)CC1)S(=O)(=O)N1CCC(Nc2ncc3cc(C(F)F)c(=O)n(C4CCCC4)c3n2)CC1] |
| II-4 | 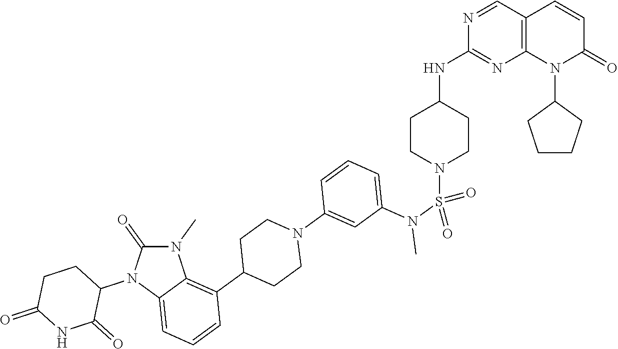 [SMILES: CN(c1cccc(N2CCC(c3cccc4c3n(C)c(=O)n4C3CCC(=O)NC3=O)CC2)c1)S(=O)(=O)N1CCC(Nc2ncc3ccc(=O)n(C4CCCC4)c3n2)CC1] |
| II-5 | 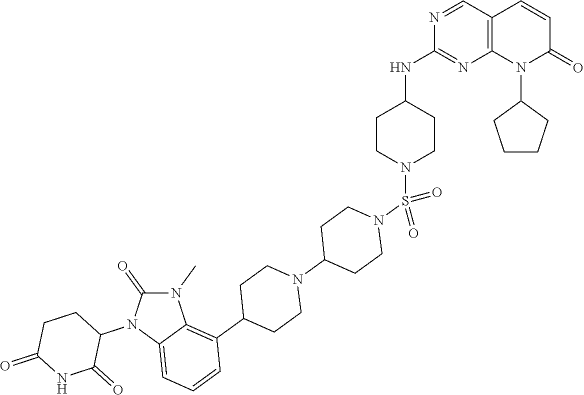 [SMILES: Cn1c(=O)n(C2CCC(=O)NC2=O)c2cccc(C3CCN(C4CCN(S(=O)(=O)N5CCC(Nc6ncc7ccc(=O)n(C8CCCC8)c7n6)CC5)CC4)CC3)c21] |
| II-5 | 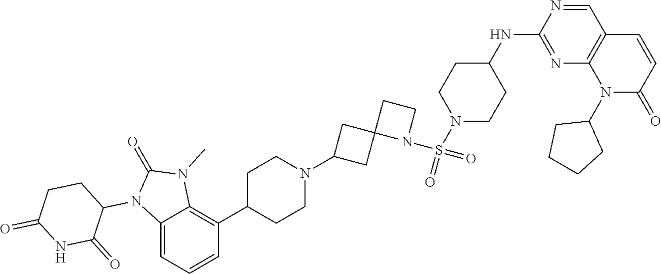 [SMILES: Cn1c(=O)n(C2CCC(=O)NC2=O)c2cccc(C3CCN(C4CC5(CCN5S(=O)(=O)N5CCC(Nc6ncc7ccc(=O)n(C8CCCC8)c7n6)CC5)C4)CC3)c21] |
| II-7 | 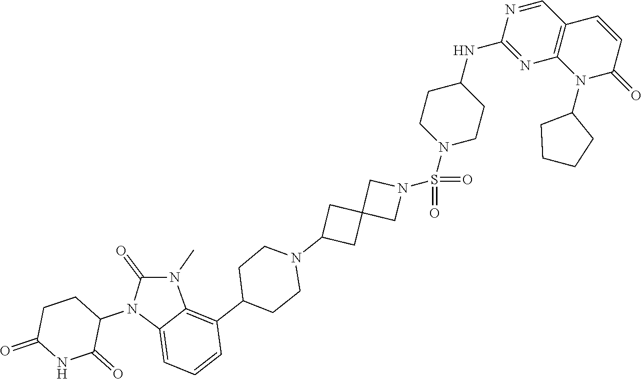 [SMILES: Cn1c(=O)n(C2CCC(=O)NC2=O)c2cccc(C3CCN(C4CC5(C4)CN(S(=O)(=O)N4CCC(Nc6ncc7ccc(=O)n(C8CCCC8)c7n6)CC4)C5)CC3)c21] |
| II-8 | 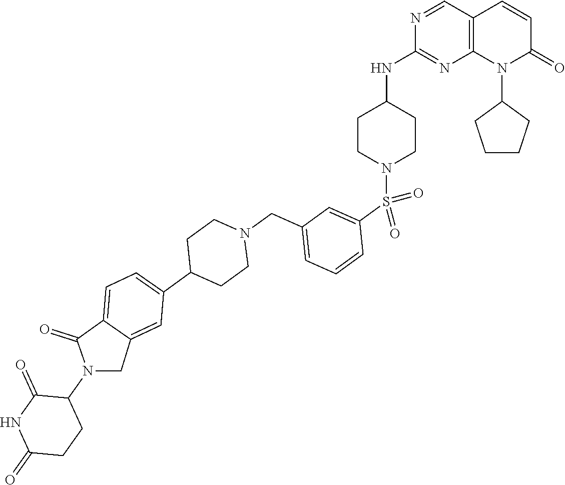 [SMILES: O=C1CCC(N2Cc3cc(C4CCN(Cc5cccc(S(=O)(=O)N6CCC(Nc7ncc8ccc(=O)n(C9CCCC9)c8n7)CC6)c5)CC4)ccc3C2=O)C(=O)N1] |
| II-9 | 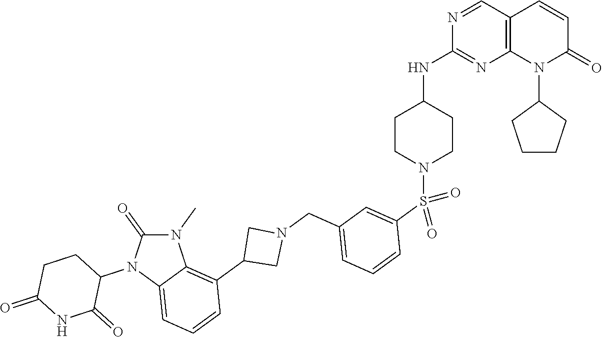 [SMILES: Cn1c(=O)n(C2CCC(=O)NC2=O)c2cccc(C3CN(Cc4cccc(S(=O)(=O)N5CCC(Nc6ncc7ccc(=O)n(C8CCCC8)c7n6)CC5)c4)C3)c21] |
| II-10 | 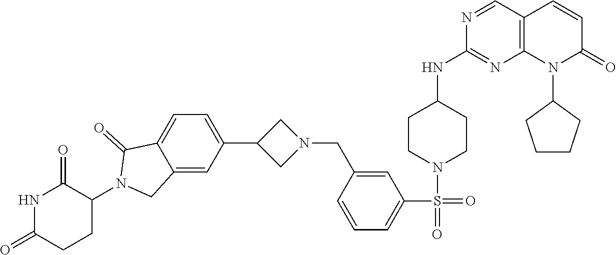 [SMILES: O=C1CCC(N2Cc3cc(C4CN(Cc5cccc(S(=O)(=O)N6CCC(Nc7ncc8ccc(=O)n(C9CCCC9)c8n7)CC6)c5)C4)ccc3C2=O)C(=O)N1] |

### EMBODIMENTS

In embodiments 1A-61 below, the present disclosure includes:
1A. In embodiment IA, provided is a compound of Formula (IA) as defined in the second aspect of the Summary.
1. In embodiment 1, provided is a compound of Formula (I) as defined in the third aspect of the Summary.
2. In embodiment 2, the compound of embodiment IA or 1, or a pharmaceutically acceptable salt thereof, is wherein R¹ is hydrogen; and R² and R³ together with the carbon atoms to which they are attached form a ring of formula (d):

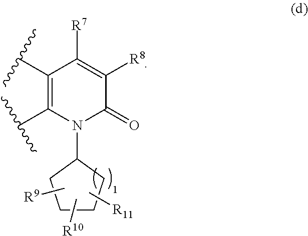

3. In embodiment 3, the compound of embodiment 1A or 1, or a pharmaceutically acceptable salt thereof. Is wherein R³ is hydrogen and R¹ is a ring of formula (c):

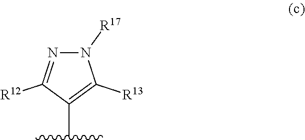

3a. In embodiment 3a, the compound of embodiment 1A, 1 or 3, or a pharmaceutically acceptable salt thereof, is wherein R¹² is hydrogen or alkyl, and R¹³ is hydrogen or haloalkyl, and R¹⁴ is cycloalkyl, cycloalkylalkyl, haloalkyl, hydroxyalkyl, aminocarbonylalkyl, aryl, heteroaryl, heterocyclyl, or heterocyclylalkyl wherein cycloalkyl, aryl, heteroaryl, and heterocyclyl are substituted with R^{g} and R^{h} independently selected from hydrogen, alkyl, halo, haloalkyl, cycloalkyl, cycloalkylalkyl, aminoalkyl, (amino)deuteroalkyl, cyano, hydroxy, alkoxy, acyl, aminocarbonyl, carboxy, amino, and optionally substituted heterocyclylalkyl; and
R² is hydrogen, alkyl, haloalkyl, halo, or cyano, preferably trifluoromethyl, cyano, or chloro.
3b. In embodiment 3b, the compound of embodiment 1A, 1, or 3, or a pharmaceutically acceptable salt thereof, is wherein R¹² and R¹³ are hydrogen or haloalkyl, and R¹⁴ is 2-hydroxy-2-methylpropyl, 1-methyl-1-CONH₂-ethyl, 2-methyl-4-(4-methylpiperazin-1-ylmethyl)phenyl, 2-methyl-4-(dimethylaminomethyl)phenyl, 2-methyl-4-(trideuteromethyl-aminomethyl)phenyl, 2-chloro-4-(methylaminomethyl)phenyl, 2-methyl-6-(isopropylamino-methyl)-50-yridine-3-yl, 2-chloro-4-(4-cyanocyclobut-1-ylaminomethyl)phenyl, 2-chloro-4-(4-hydroxycyclobut-1-ylaminomethyl)-phenyl, 2-chloro-4-(4-hydroxy-4-methylcyclohex-1-ylaminomethyl)phenyl, 2-methyl-4-(4-morpholin-4-ylmethyl)phenyl and R² is trifluoromethyl, cyano, or chloro.
4. In embodiment 4, the compound of embodiment IA or 1, or a pharmaceutically acceptable salt thereof, is wherein R³ is hydrogen and R¹ and R² together with the carbons to which they are attached form a ring of formula (f):

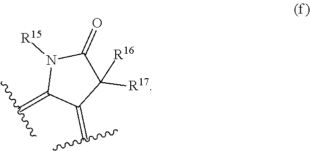

5. In embodiment 5, the compound of embodiment 1A, 1, or 2, or a pharmaceutically acceptable salt thereof, is wherein R⁹, R¹⁰, and R¹¹ are hydrogen.
6. In embodiment 6, the compound of embodiment 1A, 1, or 2, or a pharmaceutically acceptable salt thereof, is wherein R⁹, R¹⁰, and R¹¹ are independently selected from hydrogen, alkyl, haloalkyl, alkoxy, haloalkoxy, cyano, halo, and hydroxy.
7. In embodiment 7, the compound of embodiment 1A, 1, or 2, or a pharmaceutically acceptable salt thereof, is wherein R⁹ is hydrogen, R¹⁰ is hydrogen or alkyl, and R¹¹ hydrogen or hydroxy.
7a. In embodiment 7a, the compound of any one of embodiments 1A, 1, 2, and 5 to 7, or a pharmaceutically acceptable salt thereof, is wherein m is 1.
7b. In embodiment 7b, the compound of any one of embodiments 1A, 1, 2, and 5 to 7, or a pharmaceutically acceptable salt thereof, is wherein m is 2.
8. In embodiment 8, the compound of embodiment 1A, 1, or 2, or a pharmaceutically acceptable salt thereof, is wherein

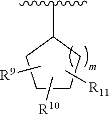

R¹⁰ in the ring of formula (d) is a group of structure:

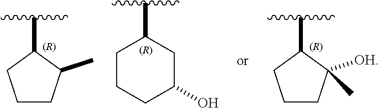

9. In embodiment 9, the compound of embodiment 1A, 1, or 2, or a pharmaceutically acceptable salt thereof, is wherein R⁹ and R¹⁰ are attached to the same carbon atom and together with the carbon atom to which they are attached form cycloalkylene or heterocyclylene.
10. In embodiment 10, the compound of any one of embodiments 1A, 1, 2 and 5 to 9, or a pharmaceutically acceptable salt thereof, is wherein R⁸ is hydrogen, halo, haloalkyl, or alkyl optionally substituted with hydroxy.
11. In embodiment 11, the compound of any one of embodiments 1A, 1, 2 and 5 to 9, or a pharmaceutically acceptable salt thereof, is wherein R⁸ is hydrogen.
12. In embodiment 12, the compound of any one of embodiments 1A, 1, 2, and 5 to 9, or a pharmaceutically acceptable salt thereof, is wherein R⁸ is haloalkyl.
13. In embodiment 13, the compound of embodiment 12, or a pharmaceutically acceptable salt thereof, is wherein R⁸ is difluoromethyl.
14. In embodiment 14, the compound of any one of embodiments 1A, 1, 2, and 5 to 9, or a pharmaceutically acceptable salt thereof, is wherein R⁸ is alkyl substituted with hydroxy.
15. In embodiment 15, the compound of embodiment 14, or a pharmaceutically acceptable salt thereof, is wherein R⁸ is 2-hydroxymethyl.
15a. In embodiment 15a, the compound of any one of embodiments 1A, 1, 2, and 5 to 9, or a pharmaceutically acceptable salt thereof, is wherein R¹ is cyano.
16. In embodiment 16, the compound of any one of embodiments 1A, 1, 2, and 5 to 15a, or a pharmaceutically acceptable salt thereof, is wherein R⁷ is hydrogen.
17. In embodiment 17, the compound of any one of embodiments 1A and 1 to 16, or a pharmaceutically acceptable salt thereof, is wherein Hy is heterocyclylene optionally substituted with one, two, or three substituents independently selected from alkyl, halo, haloalkyl, alkoxy, and hydroxy.
18. In embodiment 18, the compound of any one of embodiments 1A and 1 to 16, or a pharmaceutically acceptable salt thereof, is wherein Hy is piperidin-1,4-diyl and L is attached to the nitrogen atom of the piperidin-1,4-diyl ring of Hy.
19. In embodiment 19, the compound of any one of embodiments 1A and 1 to 16, or a pharmaceutically acceptable salt thereof, is wherein Hy is a ring of formula:

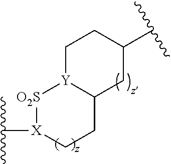

where X is CH or N and forms a bond with L; Y is CH, Cme, or N; provided at least one of X and Y is N; z is 0, 1, or 2; z′ is 0 or 1; provided at least one of z′ and z is 1; and Hy is optionally substituted with one, two, or three substituents independently selected from alkyl, halo, haloalkyl, alkoxy, and hydroxy.
20. In embodiment 20, the compound of embodiment 19, or a pharmaceutically acceptable salt thereof, is wherein Hy is a ring of formula:

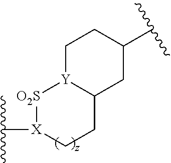

where z is 1 or 2 and Hy is optionally substituted with one, two, or three substituents independently selected from alkyl, halo, haloalkyl, alkoxy, and hydroxy.
21. In embodiment 21, the compound of embodiment 19, or a pharmaceutically acceptable salt thereof, is wherein Hy is a ring of formula:

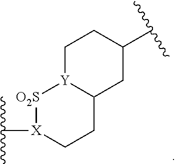

22. In embodiment 22, the compound of any one of embodiments 19 to 21, or a pharmaceutically acceptable salt thereof, is wherein X and Y are N.
23. In embodiment 23, the compound of any one of embodiments 19 to 21, or a pharmaceutically acceptable salt thereof, is wherein X is N and Y is CH.
24. In embodiment 24, the compound of any one of embodiments 19 to 21, or a pharmaceutically acceptable salt thereof, is wherein Y is N and X is CH.
25. In embodiment 25, the compound of any one of embodiments 1A and 1 to 16, or a pharmaceutically acceptable salt thereof, is wherein Hy is phenylene optionally substituted with one, two, or three substituents independently selected from alkyl, halo, haloalkyl, alkoxy, and hydroxy.
26. In embodiment 26, the compound of any one of embodiments 1A and 1 to 16, or a pharmaceutically acceptable salt thereof, is wherein Hy is spiro heterocyclylene optionally substituted with one, two, or three substituents independently selected from alkyl, halo, haloalkyl, alkoxy, and hydroxy.
27. In embodiment 27, the compound of any one of embodiments 1A and 1 to 16, or a pharmaceutically acceptable salt thereof, is wherein Hy is bridged heterocyclylene optionally substituted with one, two, or three substituents independently selected from alkyl, halo, haloalkyl, alkoxy, and hydroxy.
28. In embodiment 28, the compound of any one of embodiments 1A and 1 to 27, or a pharmaceutically acceptable salt thereof, is wherein ring A is a group of formula (a).
29. In embodiment 29, the compound of embodiment 28, or a pharmaceutically acceptable salt thereof, is wherein R⁴ and R³ are independently hydrogen or alkyl.
30. In embodiment 30, the compound of embodiment 28, or a pharmaceutically acceptable salt thereof, is wherein R⁴ and R⁵ together with the carbon to which they are attached form >C═O.
31. In embodiment 31, the compound of any one of embodiments 1A and 1 to 27, or a pharmaceutically acceptable salt thereof, is wherein ring A is a group of formula (b).
32. In embodiment 32, the compound of embodiment 31, or a pharmaceutically acceptable salt thereof, is wherein R⁶ is hydrogen.
33. In embodiment 33, the compound of embodiment 31, or a pharmaceutically acceptable salt thereof, is wherein R⁶ is alkyl, preferably methyl.
34. In embodiment 34, the compound of any one of embodiments 1A and 1 to 27, or a pharmaceutically acceptable salt thereof, is wherein ring A is a group of formula (c).
35. In embodiment 35, the compound of any one of embodiments 1A and 1 to 27, or a pharmaceutically acceptable salt thereof, is wherein ring A is:

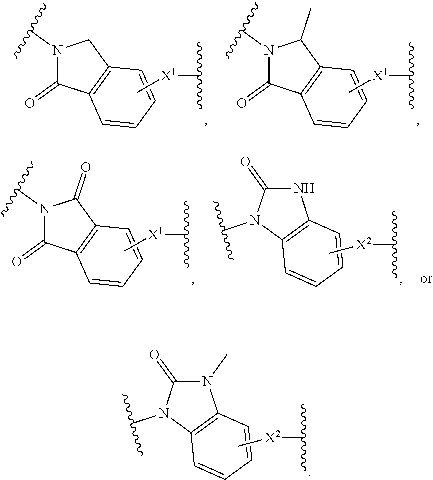

35a. In embodiment 35a, the compound of any one of embodiments 1A and 1 to 27, or a pharmaceutically acceptable salt thereof, is wherein ring A is:

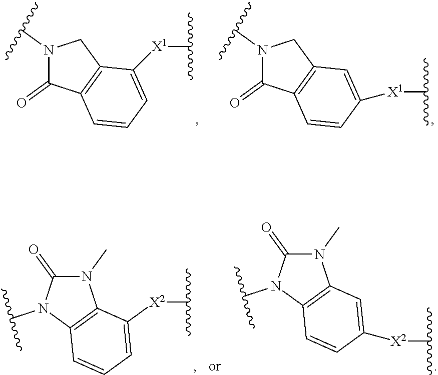

36. In embodiment 36, the compound of any one of embodiments 1A and 1 to 27, or a pharmaceutically acceptable salt thereof, is wherein ring A is:

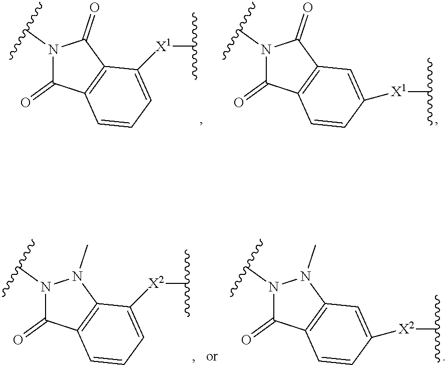

37. In embodiment 37, the compound of any one of embodiments 1A to 36, or a pharmaceutically acceptable salt thereof, is wherein X¹, X², and X³ are independently a bond.
38. In embodiment 38, the compound of any one of embodiments 1A to 36, or a pharmaceutically acceptable salt thereof, is wherein X¹, X², and X³ are independently -alkylene-, preferably methylene.
39. In embodiment 39, the compound of any one of embodiments 1A to 36, or a pharmaceutically acceptable salt thereof, is wherein X¹, X², and X³ are independently —O—.
40. In embodiment 40, the compound of any one of embodiments 1A to 36, or a pharmaceutically acceptable salt thereof, is wherein X¹, X², and X³ are independently —(O-alkylene)-.
41. In embodiment 41, the compound of any one of embodiments 1A to 36, or a pharmaceutically acceptable salt thereof, is wherein X¹, X², and X³ are independently -(alkylene-O)—.
42. In embodiment 42, the compound of any one of embodiments 1A to 36, or a pharmaceutically acceptable salt thereof, is wherein X¹, X², and X³ are independently —(NR′-alkylene)-.
43. In embodiment 43, the compound of any one of embodiments 1A to 36, or a pharmaceutically acceptable salt thereof, is wherein X¹, X², and X³ are independently -(alkylene-NR^{t})—.
44. In embodiment 44, the compound of any one of embodiments 1A to 36, or a pharmaceutically acceptable salt thereof, is wherein X¹, X², and X³ are —C≡C—
45. In embodiment 45, the compound of any one of embodiments 1A to 36, or a pharmaceutically acceptable salt thereof, is wherein X¹, X², and X³ are —NH—.
46. In embodiment 46, the compound of any one of embodiments 1A to 36, or a pharmaceutically acceptable salt thereof, is wherein X¹, X², and X³ are independently —N(alkyl)-.
47. In embodiment 47, the compound of any one of embodiments 1A to 36, or a pharmaceutically acceptable salt thereof, is wherein X¹, X², and X³ are —C(═O)—.
48. In embodiment 48, the compound of any one of embodiments 1A to 36, or a pharmaceutically acceptable salt thereof, is wherein X¹, X², and X³ are independently —NR^{u}C(═O)—.
49. In embodiment 49, the compound of any one of embodiments 1A to 36, or a pharmaceutically acceptable salt thereof, is wherein X¹, X², and X³ are independently —C(═O)NR^{v}—.
50. In embodiment 50, the compound of any one of embodiments 1A to 36, 42, 43, 46, 48, and 49, or a pharmaceutically acceptable salt thereof, is wherein R^{s}, R^{t}, R^{u}, and R^{v} are independently hydrogen or alkyl.
51. In embodiment 51, the compound of any one of embodiments 1A to 50, or a pharmaceutically acceptable salt thereof, is wherein Z⁶ is —S(O)₂—.
52. In embodiment 52, the compound of any one of embodiments 1A to 36, or a pharmaceutically acceptable salt thereof, is wherein —X¹-L-, —X²-L- and —X³-L- are independently selected from:

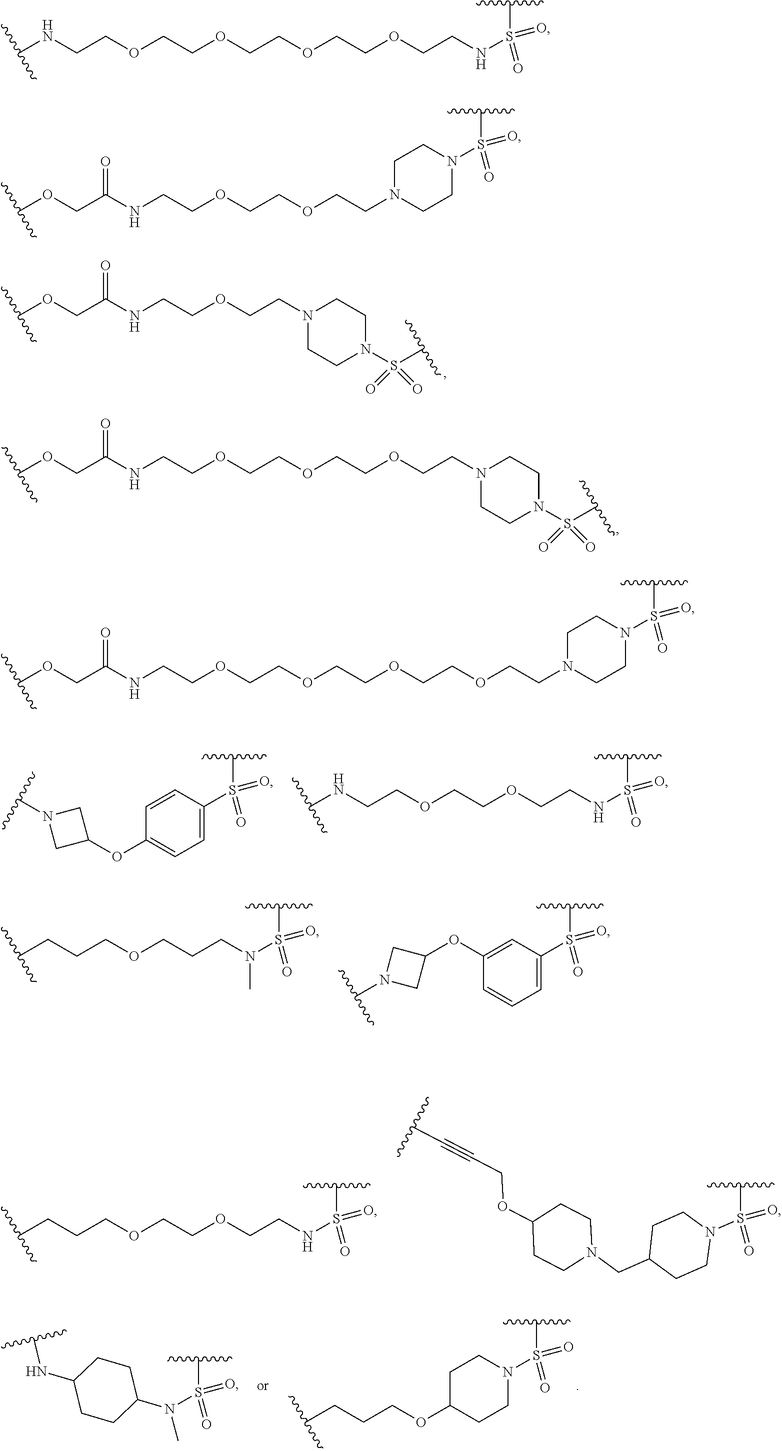

53. In embodiment 53, the compound of any one of embodiments 1A to 36, or a pharmaceutically acceptable salt thereof, is wherein Z⁵ is a bond.
54. In embodiment 54, the compound of any one of embodiments 1A to 36, or a pharmaceutically acceptable salt thereof, is wherein Z⁵ is a bond and one of Z¹ and X¹ is a bond, one of Z¹ and X² is a bond, and one of Z¹ and X³ is a bond.
55. In embodiment 55, the compound of any one of embodiments 1A to 36, or a pharmaceutically acceptable salt thereof, is wherein:
X¹, X², and X³ are independently a bond, —(O-alkylene)-, —(NR^{s}-alkylene)-, —C≡C—, —NH—, or —N(alkyl)-, where R^{s} is hydrogen or alkyl and each alkylene is optionally substituted with one or two fluoro;
Z¹ is bond, alkylene, —(CO)NR—, —(O-alkylene)ₐ-, -(alkylene-O)ₐ—, phenylene, or heterocyclylene, where each ring is optionally substituted with one or two alkyl;
Z² is a bond, alkylene, —(O-alkylene)_{b}-, -(alkylene-O)_{b}—, cycloalkylene, or heterocyclylene, where each ring is optionally substituted with one or two alkyl;
Z³ is a bond, alkylene, —C(O)NR—, —NR′(CO)—, —O—, —NR″—, cycloalkylene, phenylene, monocyclic heteroarylene, heterocyclylene, fused heterocyclylene, or spiro heterocyclylene, where each ring is optionally substituted with one or two alkyl;
Z⁴ is a bond, -(alkylene-NR″)—, —O—, —NR″—, cycloalkylene, phenylene, monocyclic heteroarylene, heterocyclylene, fused heterocyclylene, or spiro heterocyclylene, where each ring is optionally substituted with one or two alkyl;
Z⁵ is a bond; and
Z⁶ is —S(O)₂—.
56. In embodiment 56, the compound of any one of embodiments 1A to 36, or a pharmaceutically acceptable salt thereof, is wherein:
X¹, X², X³, and Z⁵ are each a bond;
Z¹ is phenylene, or heterocyclylene, where each ring is optionally substituted with one or two alkyl;
Z² is a bond, alkylene, cycloalkylene, or heterocyclylene, where each ring is optionally substituted with one or two alkyl;
Z³ is a bond, alkylene, —C(O)NR—, —NR′(CO)—, —O—, —NR″—, cycloalkylene, phenylene, monocyclic heteroarylene, heterocyclylene, fused heterocyclylene, or spiro heterocyclylene, where each ring is optionally substituted with one or two alkyl;
Z⁴ is a bond, cycloalkylene, phenylene, monocyclic heteroarylene, heterocyclylene, fused heterocyclylene, or spiro heterocyclylene, where each ring is optionally substituted with one or two alkyl; and
Z⁶ is —S(O)₂—.
57. In embodiment 57, the compound of any one of embodiments 1A to 36, or a pharmaceutically acceptable salt thereof, is wherein —X¹-L-, —X²-L- and —X³-L- are independently selected from:

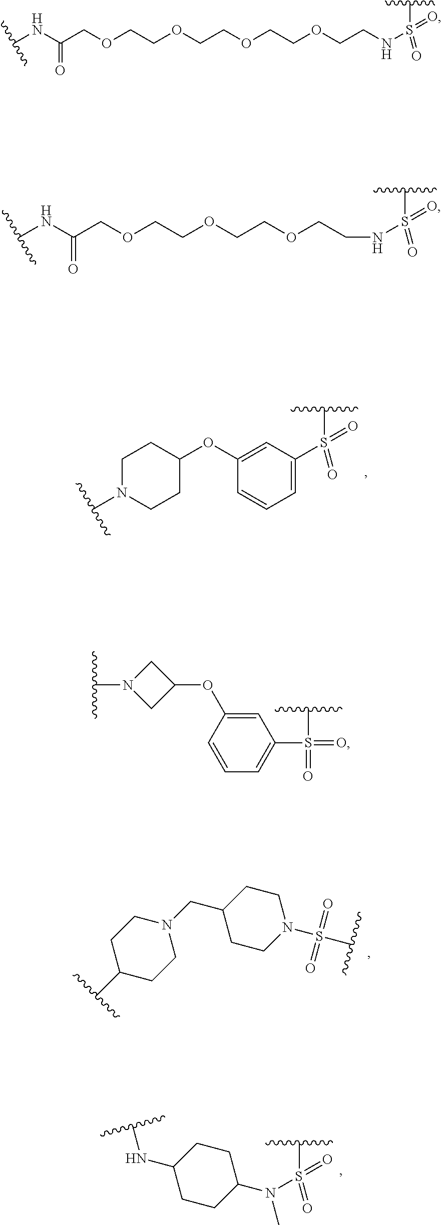 [SMILES: *NC(=O)COCCOCCOCCOCCNS(*)(=O)=O ; *NC(=O)COCCOCCOCCNS(*)(=O)=O ; *N1CCC(Oc2cccc(S(*)(=O)=O)c2)CC1 ; *N1CC(Oc2cccc(S(*)(=O)=O)c2)C1 ; *C1CCN(CC2CCN(S(*)(=O)=O)CC2)CC1 ; *NC1CCC(N(C)S(*)(=O)=O)CC1]

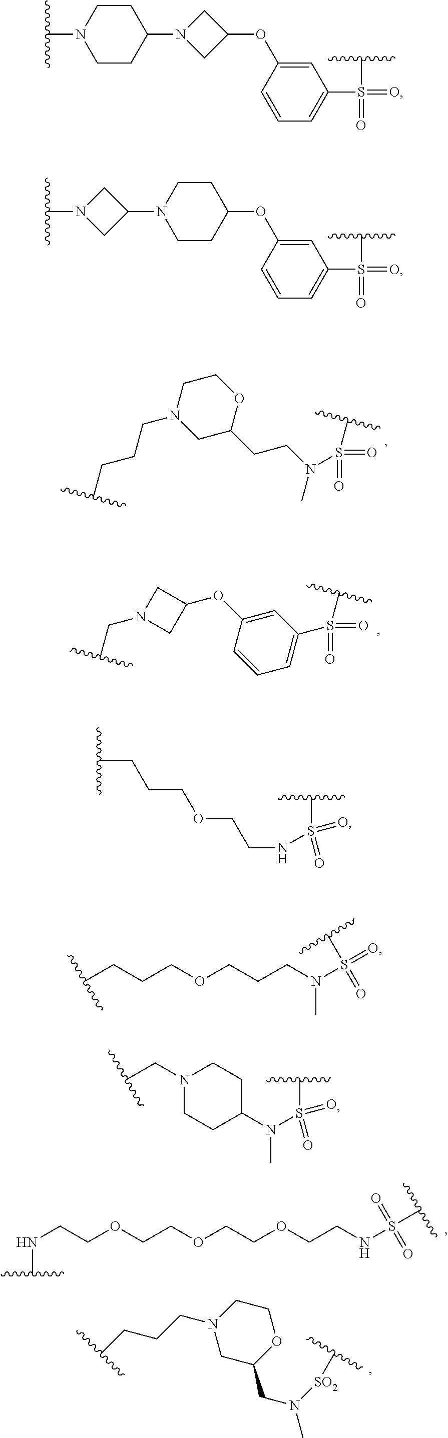

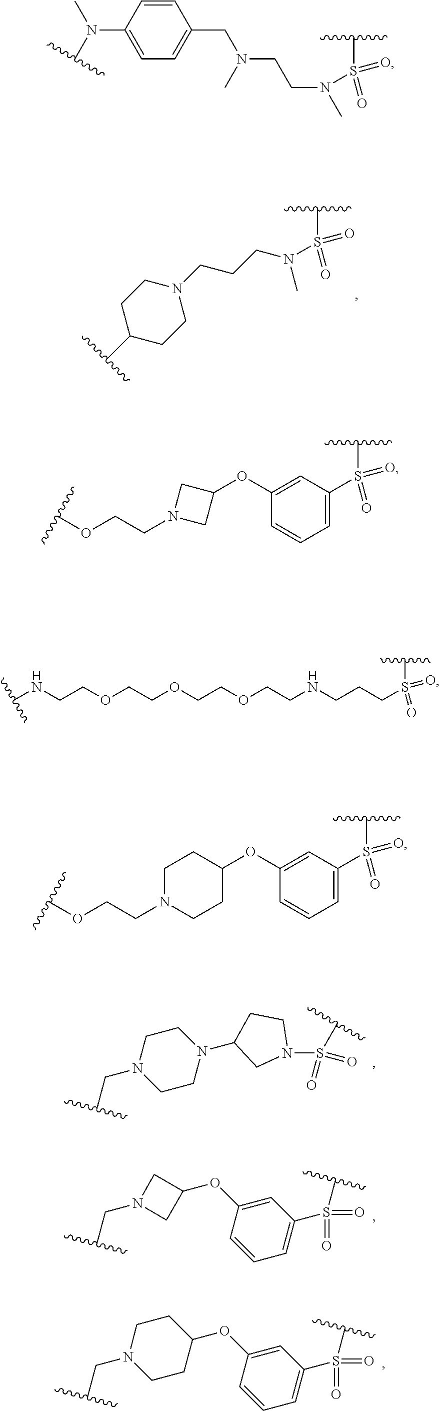

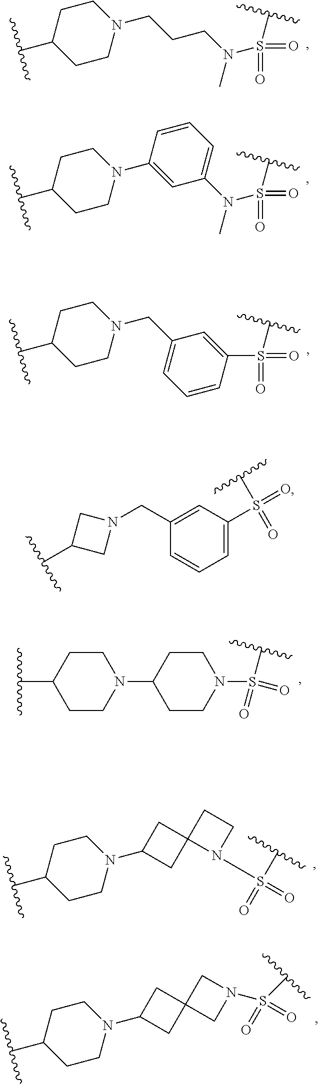

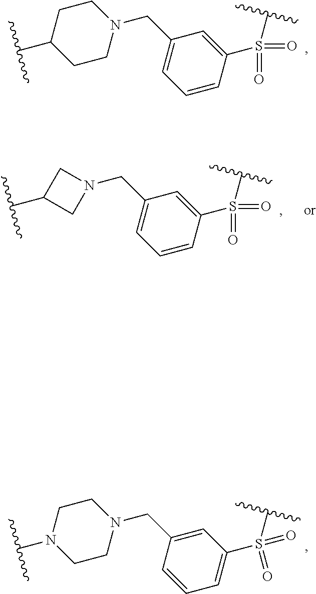 [SMILES: *C1CCN(Cc2cccc(S(*)(=O)=O)c2)CC1 ; *C1CN(Cc2cccc(S(*)(=O)=O)c2)C1 ; *N1CCN(Cc2cccc(S(*)(=O)=O)c2)CC1]

preferably, wherein —X¹-L-, —X²-L- and —X³-L- are independently:

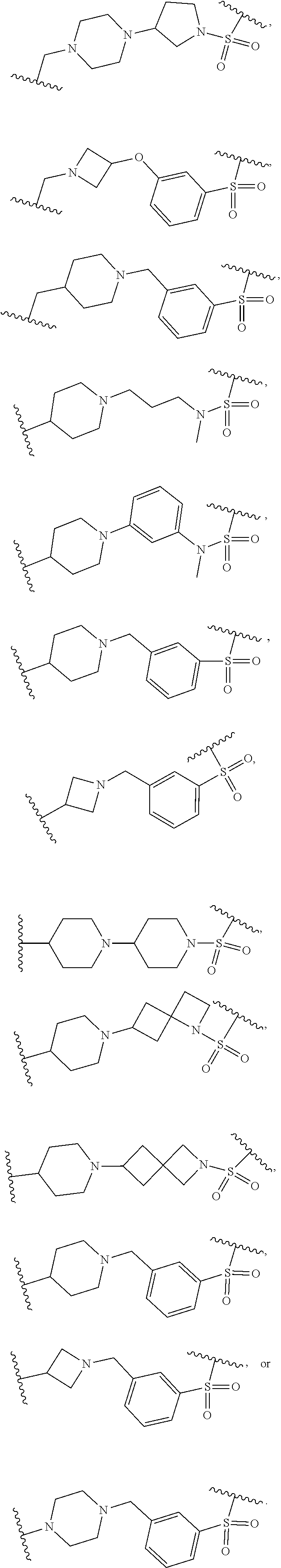

58. In embodiment 58, provided is a pharmaceutical composition comprising a compound of any one of embodiments 1A to 57, or a pharmaceutically acceptable salt thereof, and a pharmaceutically acceptable excipient.
59. In embodiment 59, provided is a method of degrading CDK2 in a cell which method comprises contacting the cell with a compound of any one of embodiments 1A to 57, or a pharmaceutically acceptable salt thereof, or with a pharmaceutical composition of embodiment 58.
60. In embodiment 60, provided is a method of treating a disease mediated by CDK2 in a patient which method comprises administering to the patient in recognized need thereof, a therapeutically effective amount of a pharmaceutical composition comprising a compound of any one of embodiments 1A to 57, or a pharmaceutically acceptable salt thereof, and a pharmaceutically acceptable excipient.
61. In embodiment 61, provided is a method of treating cancer in a patient which method comprises administering to the patient in recognized need thereof, a therapeutically effective amount a compound of any one of embodiments 1A to 57, or a pharmaceutically acceptable salt thereof, in a pharmaceutical composition comprising the compound of any one of embodiments 1A to 57 and a pharmaceutically acceptable excipient.

In further embodiments A1-A151 below, the present disclosure includes:
A1. In embodiment A1, provided is a compound of Formula (IA′) or a pharmaceutically acceptable salt is as defined in the first aspect of the Summary.
A2. In embodiment A2, the compound of embodiment A1, or a pharmaceutically acceptable salt thereof, is wherein R¹ is hydrogen; and R² and R³ together with the carbon atoms to which they are attached form a ring of formula (d1):

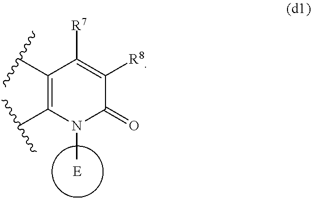

A3. In embodiment A3, the compound of embodiment A1 or A2, or a pharmaceutically acceptable salt thereof, is wherein R¹ is hydrogen; and R² and R³ together with the carbon atoms to which they are attached form a ring of formula (d):

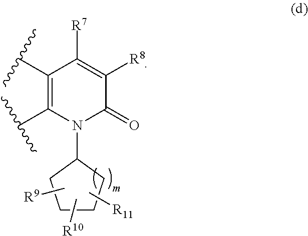

A4. In embodiment A4, the compound of embodiment A2, or a pharmaceutically acceptable thereof, is wherein ring E is bridged cycloalkyl or bicyclic cycloalkyl.
A5. In embodiment A5, the compound of embodiment A2 or A4, or a pharmaceutically acceptable salt thereof, is wherein the bridged cycloalkyl and bicyclic cycloalkyl are

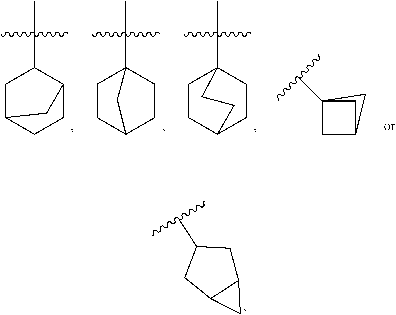

respectively, each ring optionally substituted with one or two substituents independently selected from deuterium, alkyl, halo, and haloalkyl.
A6. In embodiment A6, the compound of embodiment A1, or a pharmaceutically acceptable salt thereof, is wherein R³ is hydrogen and R¹ is a ring of formula (e):

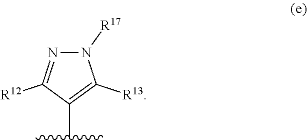

A7. In embodiment A7, the compound of embodiment A1 or A6, or a pharmaceutically acceptable salt thereof, is wherein R¹² is hydrogen or alkyl and R¹³ is hydrogen or haloalkyl, and R¹⁴ is cycloalkyl, cycloalkylalkyl, haloalkyl, hydroxyalkyl, aminocarbonylalkyl, aryl, heteroaryl, heterocyclyl, or heterocyclylalkyl wherein cycloalkyl, aryl, heteroaryl, and heterocyclyl are substituted with R^{g} and R^{h} independently selected from hydrogen, alkyl, halo, haloalkyl, cycloalkyl, cycloalkylalkyl, aminoalkyl, (amino)deuteroalkyl, cyano, hydroxy, alkoxy, acyl, aminocarbonyl, carboxy, amino, and optionally substituted heterocyclylalkyl; and
R² is hydrogen, alkyl, haloalkyl, halo, or cyano, preferably trifluoromethyl, cyano, or chloro.
A8. In embodiment A8, the compound of embodiment A1 or A6, or a pharmaceutically acceptable salt thereof, is wherein R¹² and R¹³ are independently hydrogen or haloalkyl, and R¹⁴ is 2-hydroxy-2-methylpropyl, 1-methyl-1-CONH₂-ethyl, 2-methyl-4-(4-methylpiperazin-1-ylmethyl)phenyl, 2-methyl-4-(dimethylaminomethyl)phenyl, 2-methyl-4-(trideuteromethyl-aminomethyl)phenyl, 2-chloro-4-(methylaminomethyl)phenyl, 2-methyl-6-(isopropylamino-methyl)pyridin-3-yl, 2-chloro-4-(4-cyanocyclobut-1-ylaminomethyl)phenyl, 2-chloro-4-(4-hydroxycyclobut-1-ylaminomethyl)-phenyl, 2-chloro-4-(4-hydroxy-4-methylcyclohex-1-ylaminomethyl)phenyl, or 2-methyl-4-(4-morpholin-4-ylmethyl)phenyl and R² is trifluoromethyl, cyano, or chloro.
A9. In embodiment A9, the compound of embodiment A1, or a pharmaceutically acceptable salt thereof, is wherein R³ is hydrogen and R¹ and R² together with the carbons to which they are attached form a ring of formula (f):

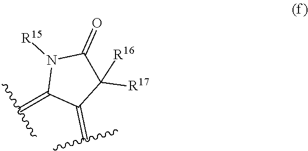

i.e., compound (IA′) is according to formula

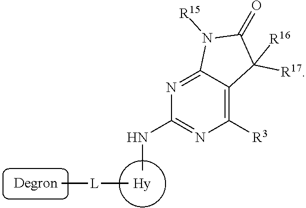

A10. In embodiment A10, the compound of any one of embodiments A1 to A3, or a pharmaceutically acceptable salt thereof, is wherein R⁹, R¹⁰, and R¹¹ are each independently hydrogen or deuterium, preferably hydrogen.
A11. In embodiment A11, the compound of any one of embodiments A1 to A3, or a pharmaceutically acceptable salt thereof, is wherein R⁹, R¹⁰, and R¹¹ are independently selected from hydrogen, deuterium, alkyl, haloalkyl, alkoxy, haloalkoxy, cyano, halo, and hydroxy.
A12. In embodiment A12, the compound of any one of embodiment A1 to A3, or a pharmaceutically acceptable salt thereof, is wherein R⁹ is hydrogen or deuterium, R¹⁰ is hydrogen or alkyl, and R″ is hydrogen or hydroxy.
A13. In embodiment A13, the compound of any one of embodiments A1 to A3 and A10 to A12, or a pharmaceutically acceptable salt thereof, is wherein m is 1.
A14. In embodiment A14, the compound of any one of embodiments A1 to A3 and A10 to A12, or a pharmaceutically acceptable salt thereof, is wherein m is 2.
A15. In embodiment A15, the compound of any one of embodiments A1 to A3, or a pharmaceutically acceptable salt thereof, is wherein

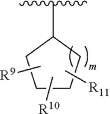

in the ring of formula (d1) and (d) is a group of structure:

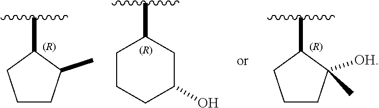

A16. In embodiment A16, the compound of any one of embodiments A1 to A3, or a pharmaceutically acceptable salt thereof, is wherein R⁹ and R¹⁰ are attached to the same carbon atom and together with the carbon atom to which they are attached form cycloalkylene or heterocyclylene.
A17. In embodiment A17, the compound of any one of embodiments A1 to A5 and A10 to A16, or a pharmaceutically acceptable salt thereof, is wherein R⁸ is hydrogen, cyano, halo, haloalkyl, or alkyl optionally substituted with hydroxy.
A18. In embodiment A18, the compound of any one of embodiments A1 to A5 and A10 to A17, or a pharmaceutically acceptable salt thereof, is wherein R⁸ is hydrogen, fluoro, chloro, difluoromethyl, trifluoromethyl, methyl, or hydroxymethyl.
A19. In embodiment A19, the compound of any one of embodiments A1 to A5, and A10 to A17, or a pharmaceutically acceptable salt thereof, is wherein R⁸ is hydrogen.
A20. In embodiment A20, the compound of any one of embodiments A1 to A5 and A10 to A17, or a pharmaceutically acceptable salt thereof, is wherein R⁸ is haloalkyl.
A21. In embodiment A21, the compound of embodiment A20, or a pharmaceutically acceptable salt thereof, is wherein R⁸ is difluoromethyl.
A22. In embodiment A22, the compound of any one of embodiments A1 to A5 and A10 to A17, or a pharmaceutically acceptable salt thereof, is wherein R⁸ is alkyl substituted with hydroxy.
A23. In embodiment A23, the compound of embodiment A22, or a pharmaceutically acceptable salt thereof, is wherein R⁸ is 2-hydroxymethyl.
A24. In embodiment A24, the compound of any one of embodiments A1 to A5 and A10 to A17, or a pharmaceutically acceptable salt thereof, or a pharmaceutically acceptable salt thereof, is wherein R⁸ is cyano.
A25. In embodiment A25, the compound of any one of embodiments A1 to A5 and A10 to A24, or a pharmaceutically acceptable salt thereof, or a pharmaceutically acceptable salt thereof, is wherein R⁷ is hydrogen.
A26. In embodiment A26, the compound of any one of embodiments A1 to A25, or a pharmaceutically acceptable salt thereof, is wherein Hy is heterocyclylene, phenylene, or spiro heterocyclylene, wherein each of aforementioned rings is optionally substituted with one or two substituents independently selected from deuterium, alkyl, halo, haloalkyl, alkoxy, and hydroxy.
A27. In embodiment A27, the compound of any one of embodiments A1 to A26, or a pharmaceutically acceptable salt thereof, is wherein Hy is heterocyclylene optionally substituted with one or two substituents independently selected from deuterium, alkyl, halo, haloalkyl, alkoxy, and hydroxy.
A28. In embodiment A28, the compound of any one of embodiments A1 to A27, or a pharmaceutically acceptable salt thereof, is wherein Hy is piperidin-1,4-diyl optionally substituted with deuterium, methyl, fluoro, methoxy, or hydroxy and L is attached to the nitrogen atom of the piperidin-1,4-diyl ring of Hy.
A29. In embodiment A29, the compound of any one of embodiments A1 to A28, or a pharmaceutically acceptable salt thereof, is wherein Hy is:

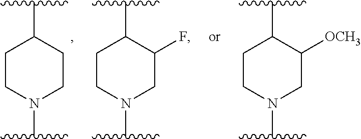

where the N atom of the piperidine ring is attached to L.
A30. In embodiment A30, the compound of any one of embodiments A1 to A29, or a pharmaceutically acceptable salt thereof, is wherein Hy is:

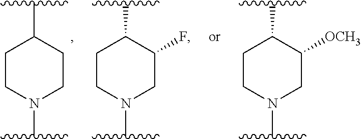

where the N atom of the piperidine ring is attached to L.
A31. In embodiment A31, the compound of any one of embodiments A1 to A25, or a pharmaceutically acceptable salt thereof, is wherein Hy is a ring of formula:

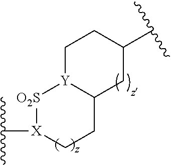

where X is CH or N and forms a bond with L; Y is CH, CMe, or N; provided at least one of X and Y is N; z is 0, 1, or 2; z′ is 0 or 1; provided at least one of z′ and z is 1; and Hy is optionally substituted with one, two, or three substituents independently selected from alkyl, halo, haloalkyl, alkoxy, and hydroxy.
A32. In embodiment A32, the compound of embodiment A31, or a pharmaceutically acceptable salt thereof, is wherein Hy is a ring of formula:

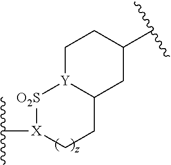

where z is 1 or 2 and Hy is optionally substituted with one, two, or three substituents independently selected from alkyl, halo, haloalkyl, alkoxy, and hydroxy.
A33. In embodiment A33, the compound of embodiment A31, or a pharmaceutically acceptable salt thereof, is wherein Hy is a ring of formula:

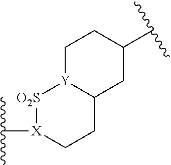

A34. In embodiment A34, the compound of any one of embodiments A31 to A33, or a pharmaceutically acceptable salt thereof, is wherein X and Y are each N.
A35. In embodiment A35, the compound of any one of embodiments A31 to A33, or a pharmaceutically acceptable salt thereof, is wherein X is N and Y is CH.
A36. In embodiment A36, the compound of any one of embodiments A31 to A33, or a pharmaceutically acceptable salt thereof, is wherein Y is N and X is CH.
A37. In embodiment A37, the compound of any one of embodiments A1 to A26, or a pharmaceutically acceptable salt thereof, is wherein Hy is phenylene optionally substituted with one or two substituents independently selected from alkyl, halo, haloalkyl, alkoxy, and hydroxy.
A38. In embodiment A38, the compound of any one of embodiments A1 to A26, or a pharmaceutically acceptable salt thereof, is wherein Hy is spiro heterocyclylene optionally substituted with one or two substituents independently selected from alkyl, halo, haloalkyl, alkoxy, and hydroxy.
A39. In embodiment A39, the compound of any one of embodiments A1 to A25, or a pharmaceutically acceptable salt thereof, is wherein Hy is bridged heterocyclylene optionally substituted with one or two substituents independently selected from alkyl, halo, haloalkyl, alkoxy, and hydroxy.
A40. In embodiment A40, the compound of any one of embodiments A1 to A39, or a pharmaceutically acceptable salt thereof, is wherein the Degron is an E3 ligase ligand of formula (i):

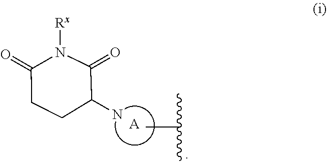

A41. In embodiment A41, the compound of any one of embodiments A1 to A40, or a pharmaceutically acceptable salt thereof, is wherein ring A of the E3 ligase ligand of formula (i) is a group of formula (a):

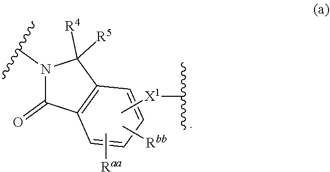

A42. In embodiment A42, the compound of any one of embodiments A1 to A41, or a pharmaceutically acceptable salt thereof, is wherein R⁴ and R⁵ are independently hydrogen or alkyl.
A43. In embodiment A43, the compound of any one of embodiments A1 to A41, or a pharmaceutically acceptable salt thereof, is wherein R⁴ and R⁵ are independently hydrogen.
A44. In embodiment A44, the compound of any one of embodiments A1 to 41, or a pharmaceutically acceptable salt thereof, is wherein R⁴ is hydrogen and R⁵ is methyl.
A45. In embodiment A45, the compound of any one of embodiments A1 to A41, or a pharmaceutically acceptable salt thereof, is wherein R⁴ and R⁵ together with the carbon to which they are attached form >C═O.
A46. In embodiment A46, the compound of any one of embodiments A1 to A40, or a pharmaceutically acceptable salt thereof, is wherein the ring A of the E3 ligase ligand of formula (i) is a group of formula (b):

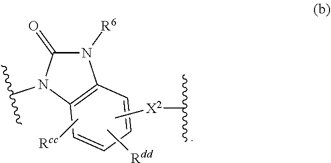

A47. In embodiment A47, the compound of any one of embodiments A1 to A40 and A46, or a pharmaceutically acceptable salt thereof, is wherein R⁶ is hydrogen.
A48. In embodiment A48, the compound of any one of embodiments A1 to A40 and A46, or a pharmaceutically acceptable salt thereof, wherein R⁶ is alkyl, preferably methyl.
A49. In embodiment A49, the compound of any one of embodiments A1 to A40, or a pharmaceutically acceptable salt thereof, is wherein ring A of the E3 ligase ligand of formula (i) is a group of formula (c):

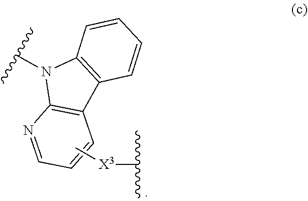

A50. In embodiment A50, the compound of any one of embodiments A1 to A40, or a pharmaceutically acceptable salt thereof, is wherein ring A of the E3 ligase ligand of formula (i) is:

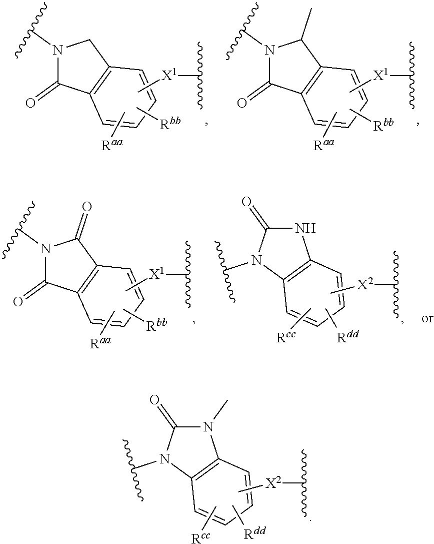

A51. In embodiment A51, the compound of any one of embodiments A1 to A40, or a pharmaceutically acceptable salt thereof, is wherein ring A of the E3 ligase ligand of formula (i) is:

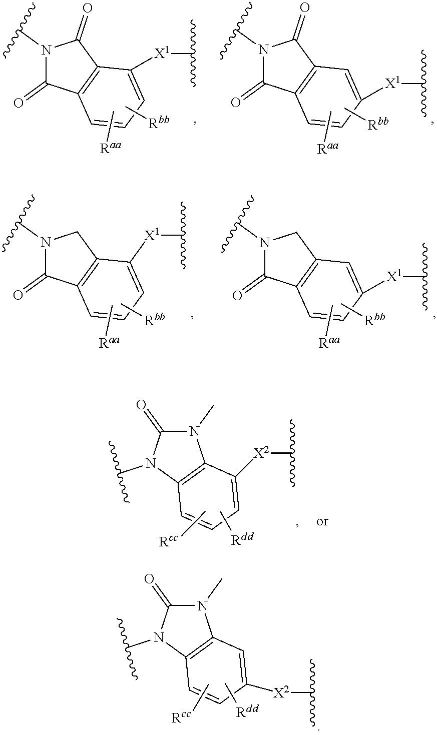

A52. In embodiment A52, the compound of any one of embodiments A1 to A40, or a pharmaceutically acceptable salt thereof, is wherein ring A of the E3 ligase ligand of formula (i) is:

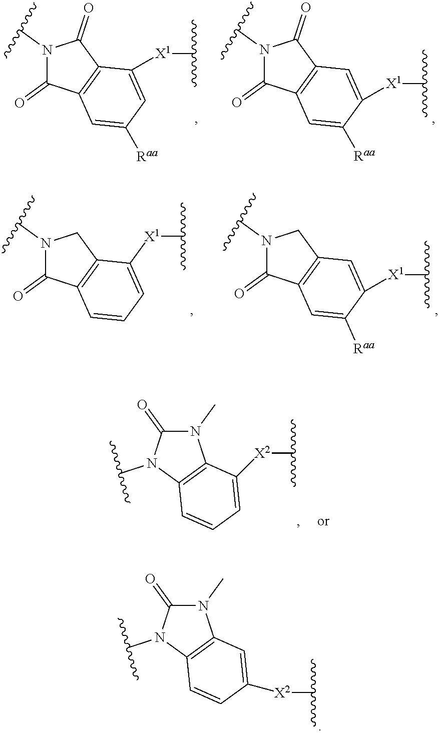

A52a. In embodiment A52a, the compound of any one of embodiments A1 to A40, or a pharmaceutically acceptable salt thereof, is wherein ring A of the E3 ligase ligand of formula (i) is:

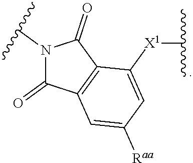

A53. In embodiment A53, the compound of any one of embodiments A1 to A40, or a pharmaceutically acceptable salt thereof, is wherein ring A of the E3 ligase ligand of formula (i) is:

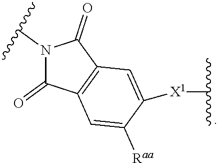

A54. In embodiment A54, the compound of any one of embodiments A1 to A40, or a pharmaceutically acceptable salt thereof, is wherein ring A of the E3 ligase ligand of formula (i) is:

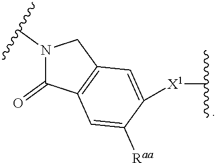

A55. In embodiment A55, the compound of any one of embodiments A1 to A40, or a pharmaceutically acceptable salt thereof, is wherein ring A of the E3 ligase ligand of formula (i) is:

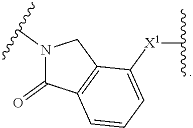

A56. In embodiment A56, the compound of any one of embodiments A1 to A40, or a pharmaceutically acceptable salt thereof, is wherein ring A of the E3 ligase ligand of formula (i) is:

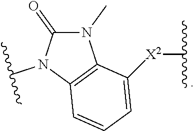

A57. In embodiment A57, the compound of any one of embodiments A1 to A40, or a pharmaceutically acceptable salt thereof, is wherein ring A of the E3 ligase ligand of formula (i) is:

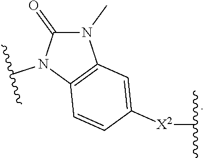

A58. In embodiment A58, the compound of any one of embodiments A1 to A54, or a pharmaceutically acceptable salt thereof, is wherein R^{aa}, R^{bb}, R^{cc}, and R^{dd} are independently selected from hydrogen, alkyl, alkoxy, halo, haloalkyl, and haloalkoxy.
A59. In embodiment A59, the compound of any one of embodiments A1 to A54, or a pharmaceutically acceptable salt thereof, is wherein R^{aa}, R^{bb}, R^{cc}, and R^{dd} are independently selected from hydrogen, alkyl, alkoxy, halo, haloalkyl, and cyano.
A60. In embodiment A60, the compound of any one of embodiments A1 to A54, or a pharmaceutically acceptable salt thereof, is wherein R^{aa}, R^{bb}, R^{cc}, and R^{dd} are independently selected from hydrogen, methyl, methoxy, ethoxy, fluoro, trifluoromethyl, difluoromethyl, and trifluoromethoxy.
A61. In embodiment A61, the compound of any one of embodiments A1 to A54, or a pharmaceutically acceptable salt thereof, is wherein R^{aa}, R^{bb}, R^{cc}, and R^{dd} are independently selected from hydrogen and methyl.
A62. In embodiment A62, the compound of any one of embodiments A1 to A54, or a pharmaceutically acceptable salt thereof, is wherein R^{aa}, R^{bb}, R^{cc}, and R^{d} are independently selected from hydrogen and methoxy.
A63. In embodiment A63, the compound of any one of embodiments A1 to A54, or a pharmaceutically acceptable salt thereof, is wherein R^{aa}, R^{bb}, R^{cc}, and R^{d} are independently selected from hydrogen and fluoro.
A64. In embodiment A64, the compound of any one of embodiments A1 to A54, or a pharmaceutically acceptable salt thereof, is wherein R^{aa}, R^{bb}, R^{cc}, and R^{d} are independently selected from hydrogen, trifluoromethyl, and difluoromethyl.
A65. In embodiment A65, the compound of any one of embodiments A1 to A54, or a pharmaceutically acceptable salt thereof, is wherein R^{aa}, R^{bb}, R^{cc}, and R^{d} are independently selected from hydrogen and trifluoromethoxy.
A66. In embodiment A66, the compound of any one of embodiments A1 to A54, or a pharmaceutically acceptable salt thereof, is wherein R^{aa}, R^{bb}, R^{cc}, and R^{d} are independently selected from hydrogen, fluoro, and trifluoromethyl.
A67. In embodiment A67, the compound of any one of embodiments A1 to A39, or a pharmaceutically acceptable salt thereof, is wherein the Degron is an E3 ligase ligand of formula (ii):

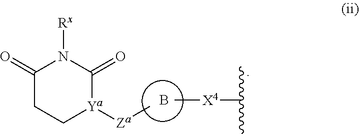

A68. In embodiment A68, the compound of any one of embodiments A1 to A39 and A67, or a pharmaceutically acceptable salt thereof, is wherein Y^{a} is CH.
A69. In embodiment A69, the compound of any one of embodiments A1 to A39 and A67, or a pharmaceutically acceptable salt thereof, is wherein Y^{a} is N.
A70. In embodiment A70, the compound of any one of embodiments A1 to A39, and A67-A69, or a pharmaceutically acceptable salt thereof, is wherein Z^{a} is a bond, —NH—, O, or —NHC(O)—.
A71. In embodiment A72, the compound of any one of embodiments A1 to A39, and A67-A69, or a pharmaceutically acceptable salt thereof, is wherein Z^{a} is a bond, —NH—, or —NHC(O)—.
A72. In embodiment A72, the compound of any one of embodiments A1 to A39, and A67-A69, or a pharmaceutically acceptable salt thereof, is wherein Z^{a} is a bond.
A73. In embodiment A73, the compound of any one of embodiments A1 to A39, and A67-A69, or a pharmaceutically acceptable salt thereof, is wherein Z^{a} is —NH—, or —NHC(O)—.
A74. In embodiment A74, the compound of any one of embodiments A1 to A39, and A67-A69, or a pharmaceutically acceptable salt thereof, is wherein Z^{a} is —NH—.
A74a. In embodiment A74a, the compound of any one of embodiments A1 to A39, and A67-A69, or a pharmaceutically acceptable salt thereof, is wherein Z^{a} is —NHC(O)—.
A75. In embodiment A75, the compound of any one of embodiments A1 to A39, and A67-A74a, or a pharmaceutically acceptable salt thereof, is wherein ring B is phenylene substituted with R^{ee} and R^{ff} independently selected from hydrogen, alkyl, alkoxy, halo, haloalkyl, haloalkoxy, and cyano.
A76. In embodiment A76, the compound of any one of embodiments A1 to A39, and A67-A74a, or a pharmaceutically acceptable salt thereof, is wherein ring B is cyclylaminylene substituted with R^{ee} and R^{ff} independently selected from hydrogen, alkyl, alkoxy, halo, haloalkyl, haloalkoxy, and cyano.
A77. In embodiment A77, the compound of any one of embodiments A1 to A39, and A67-A74a, or a pharmaceutically acceptable salt thereof, is wherein ring B is 5- or 6-membered monocyclic heteroarylene or a 9- or 10-membered fused bicyclic heteroarylene, wherein each heteroarylene ring contains one to three nitrogen ring atoms and each ring is substituted with R^{ee} and R^{ff} independently selected from hydrogen, alkyl, alkoxy, halo, haloalkyl, haloalkoxy, and cyano.
A78. In embodiment A78, the compound of any one of embodiments A1 to A39, and A67-A74a, or a pharmaceutically acceptable salt thereof, is wherein ring B is 5- or 6-membered monocyclic heteroarylene containing one or two nitrogen ring atoms substituted with R^{ee} and R^{ff} independently selected from hydrogen, alkyl, alkoxy, halo, haloalkyl, haloalkoxy, and cyano.
A79. In embodiment A79, the compound of any one of embodiments A1 to A39, and A67-A74a, or a pharmaceutically acceptable salt thereof, is wherein ring B is a 9- or 10-membered fused bicyclic heteroarylene containing one to three nitrogen ring atoms and substituted with R^{ee} and R^{ff} independently selected from hydrogen, alkyl, alkoxy, halo, haloalkyl, haloalkoxy, and cyano.
A80. In embodiment A80, the compound of any one of embodiments A1 to A39, and A67-A74a, or a pharmaceutically acceptable salt thereof, is wherein ring B is a 9- or 10-membered fused bicyclic heteroarylene containing two nitrogen ring atoms and substituted with R^{ee} and R^{ff} independently selected from hydrogen, alkyl, alkoxy, halo, haloalkyl, haloalkoxy, and cyano.
A81. In embodiment A81, the compound of any one of embodiments A1 to A39, A67-A69, and A75 to A80, or a pharmaceutically acceptable salt thereof, is wherein the E3 ligase ligand of formula (ii) is:

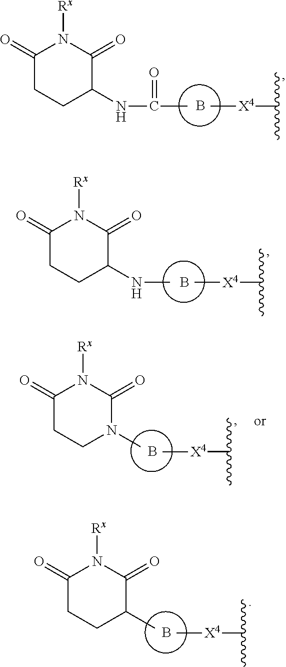

A82. In embodiment A82, the compound of any one of embodiments A1 to A39, and A67-A69, and A81, or a pharmaceutically acceptable salt thereof, is wherein the E3 ligase ligand of formula (ii) is:

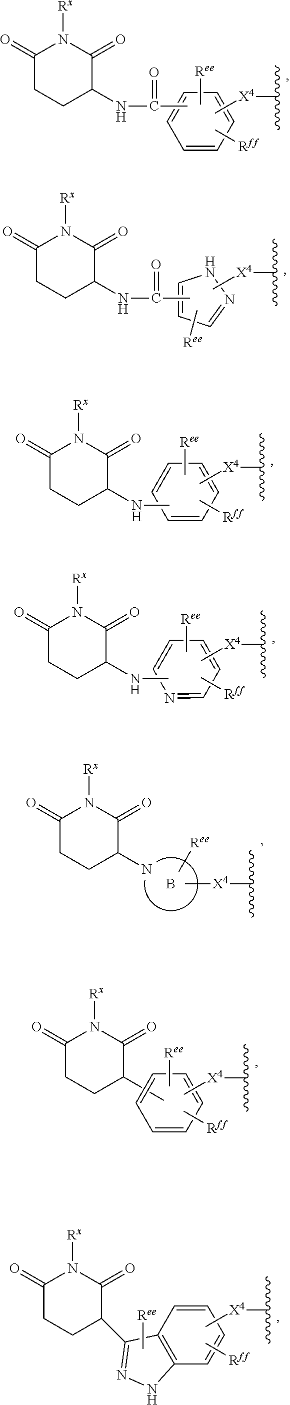

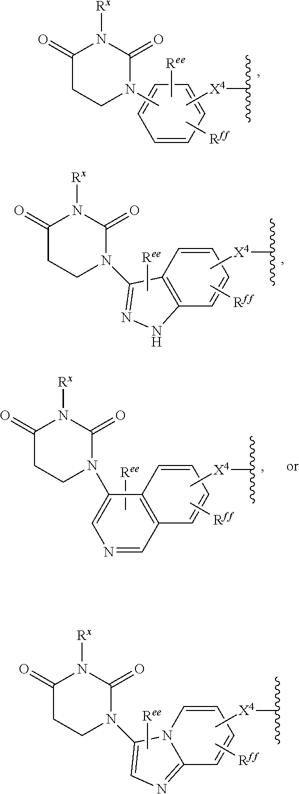

where ring B is cyclylaminylene.
A83. In embodiment A83, the compound of any one of embodiments A1 to A39, A67-A69, and A81, or a pharmaceutically acceptable salt thereof, is wherein the E3 ligase ligand of formula (ii) is:

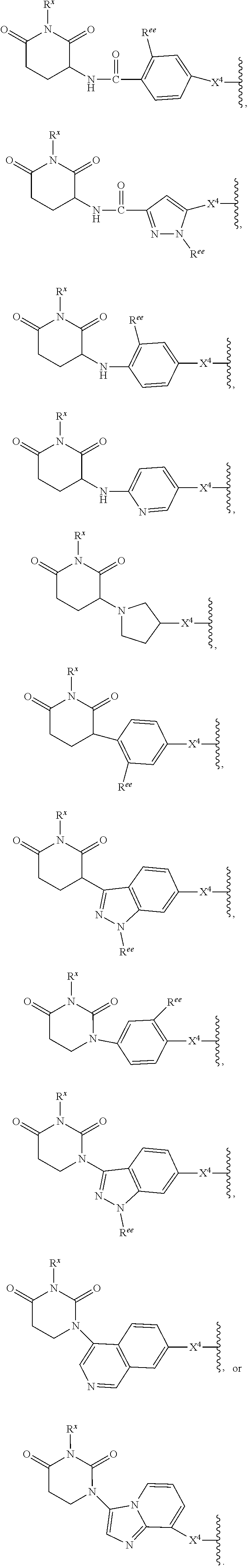

A84. In embodiment A84, the compound of any one of embodiments A1 to A39, A67 to A83, or a pharmaceutically acceptable salt thereof, is wherein each R^{ee} and R^{ff} are independently selected from hydrogen, alkyl, alkoxy, halo, haloalkyl, and haloalkoxy.
A85. In embodiment A85, the compound of any one of embodiments A1 to A39 and A67 to A83, or a pharmaceutically acceptable salt thereof, is wherein R^{ee} and R^{ff} are independently selected from hydrogen, alkyl, alkoxy, halo, haloalkyl, and cyano.
A86. In embodiment A86, the compound of any one of embodiments A1 to A39 and A67 to A83, or a pharmaceutically acceptable salt thereof, is wherein R^{ee} and R^{ff} are independently selected from hydrogen, methyl, methoxy, ethoxy, fluoro, chloro, trifluoromethyl, difluoromethyl, and trifluoromethoxy.
A87. In embodiment A87, the compound of any one of embodiments A1 to A39 and A67 to A83, or a pharmaceutically acceptable salt thereof, is wherein R^{ee} and R^{ff} are independently selected from hydrogen and methyl.
A88. In embodiment A88, the compound of any one of embodiments A1 to A39 and A67 to A83, or a pharmaceutically acceptable salt thereof, is wherein R^{ee} and R^{ff} are independently selected from hydrogen and methoxy.
A89. In embodiment A89, the compound of any one of embodiments A1 to A39 and A67 to A82, or a pharmaceutically acceptable salt thereof, is wherein R^{ee} and R^{ff} are independently selected from hydrogen, methyl, chloro, and fluoro.
A90 In embodiment A90, the compound of any one of embodiments A1 to A39 and A67 to A83, or a pharmaceutically acceptable salt thereof, is wherein R^{ee} and R^{ff} are independently selected from hydrogen, trifluoromethyl, and difluoromethyl.
A91. In embodiment A91, the compound of any one of embodiments A1 to A39 and A67 to A83, or a pharmaceutically acceptable salt thereof, is wherein R^{ee} and R^{ff} are independently selected from hydrogen and trifluoromethoxy.
A92. In embodiment A92, the compound of any one of embodiments A1 to A39 and A67 to A83, or a pharmaceutically acceptable salt thereof, is wherein R^{ee} and R^{ff} are independently selected from hydrogen, chloro, fluoro, and trifluoromethyl.
A93. In embodiment A93, the compound of any one of embodiments A1 to A39 and A67 to A83, or a pharmaceutically acceptable salt thereof, is wherein R^{ee} and R^{ff} are independently hydrogen.
A94. In embodiment A94, the compound of any one of embodiments A1 to A39 and A67 to A83, or a pharmaceutically acceptable salt thereof, is wherein R^{ee} and R^{ff} are independently chloro.
A95. In embodiment A95, the compound of any one of embodiments A1 to A39 and A67 to A83, or a pharmaceutically acceptable salt thereof, is wherein R^{ee} and R^{ff} are independently fluoro.
A96. In embodiment A96, the compound of any one of embodiments A1 to A39 and A67 to A83, or a pharmaceutically acceptable salt thereof, is wherein R^{ee} and R^{ff} are independently trifluoromethyl.
A97. In embodiment A97, the compound of any one of embodiments A1 to A96, or a pharmaceutically acceptable salt thereof, is wherein X¹, X², X³, and X⁴ are independently a bond.
A98. In embodiment A98, the compound of any one of embodiments A1 to A96, or a pharmaceutically acceptable salt thereof, is wherein X¹, X², X³, and X⁴ are independently -alkylene-, preferably methylene.
A99. In embodiment A99, the compound of any one of embodiments A1 to A96, or a pharmaceutically acceptable salt thereof, is wherein X¹, X², X³, and X⁴ are independently —O—.
A100. In embodiment A100, the compound of any one of embodiments A1 to A96, or a pharmaceutically acceptable salt thereof, is wherein X¹, X², X³, and X⁴ are independently —(O-alkylene)-.
A101. In embodiment A101, the compound of any one of embodiments A1 to A96, or a pharmaceutically acceptable salt thereof, is wherein X¹, X², X³, and X⁴ are independently -(alkylene-O)—.
A102. In embodiment A102, the compound of any one of embodiments A1 to A96, or a pharmaceutically acceptable salt thereof, is wherein X¹, X², X³, and X⁴ are independently —(NR′-alkylene)-.
A103. embodiment A103, the compound of any one of embodiments A1 to A96, or a pharmaceutically acceptable salt thereof, is wherein X¹, X², X³, and X⁴ are independently -(alkylene-NR^{t})—.
A104. In embodiment A104, the compound of any one of embodiments A1 to A96, or a pharmaceutically acceptable salt thereof, is wherein X¹, X², X³, and X⁴ are —C≡C—.
A105. In embodiment A105, the compound of any one of embodiments A1 to A96, or a pharmaceutically acceptable salt thereof, is wherein X¹, X², X³, and X⁴ are —NH—.
A106. In embodiment A106, the compound of any one of embodiments A1 to A96, or a pharmaceutically acceptable salt thereof, is wherein X¹, X², X³, and X⁴ are independently —N(alkyl)-.
A107. In embodiment A107, the compound of any one of embodiments A1 to A96, or a pharmaceutically acceptable salt thereof, is wherein X¹, X², X³, and X⁴ are —C(═O)—.
A108. In embodiment A108, the compound of any one of embodiments A1 to A96, or a pharmaceutically acceptable salt thereof, is wherein X¹, X², X³, and X⁴ are independently —NR^{u}C(═O)—.
A109. In embodiment A109, the compound of any one of embodiments A1 to A96, or a pharmaceutically acceptable salt thereof, is wherein X¹, X², X³, and X⁴ are independently —C(═O)NR^{v}—.
A110. In embodiment A110, the compound of any one of embodiments A1 to A96, A102, A103, A108, and A109, or a pharmaceutically acceptable salt thereof, is wherein R^{s}, R^{t}, R^{u}, and R^{v} are independently hydrogen or alkyl.
A111. In embodiment A111, the compound of any one of embodiments A1 to A110, or a pharmaceutically acceptable salt thereof, is wherein Z⁶ is —S(O)₂—.
A112. In embodiment A112, the compound of any one of embodiments A1 to A96, or a pharmaceutically acceptable salt thereof, is wherein —X¹-L-, —X²-L-, —X³-L- and —X⁴-L- are independently selected from:

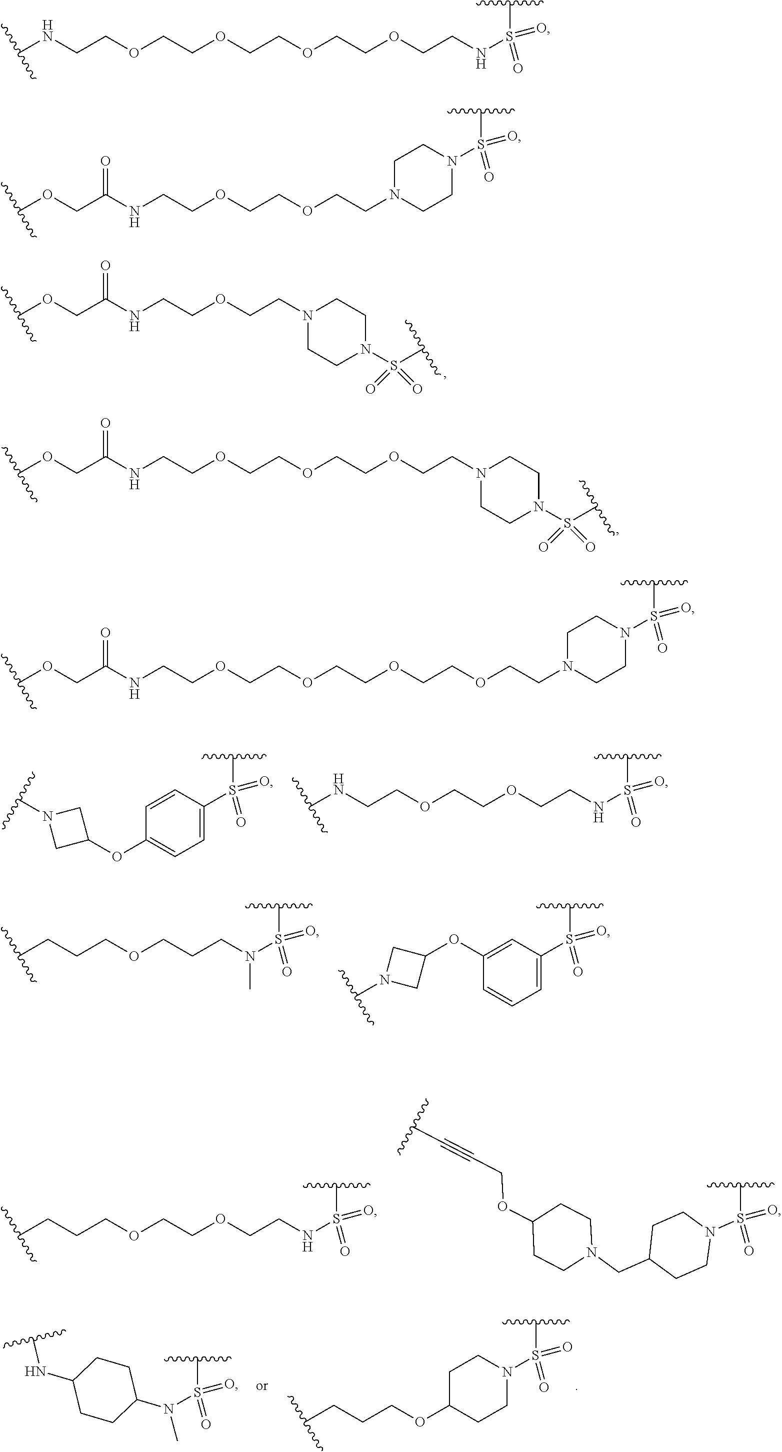

A113. In embodiment A113, the compound of any one of embodiments A1 to A11, or a pharmaceutically acceptable salt thereof, is wherein Z⁵ is a bond.
A114. In embodiment A114, the compound of any one of embodiments A1 to A96, A11, and A13, or a pharmaceutically acceptable salt thereof, is wherein Z⁵ is a bond and one of Z¹ and X¹ is a bond, one of Z¹ and X² is a bond, one of Z¹ and X³, and one of Z¹ and X⁴ is a bond.
A115. In embodiment A115, the compound of any one of embodiments A1 to A96, or a pharmaceutically acceptable salt thereof, is wherein:
X¹, X², X³, and X⁴ are independently a bond, —(O-alkylene)-, —(NR^{s}-alkylene)-, —C≡C—, —NH—, or —N(alkyl)-, where R^{s} is hydrogen or alkyl and each alkylene is optionally substituted with one or two fluoro;
Z¹ is a bond, alkylene, —(CO)NR—, —(O-alkylene)ₐ-, -(alkylene-O)ₐ—, phenylene, or heterocyclylene, where each ring is optionally substituted with one or two alkyl;
Z² is a bond, alkylene, —(O-alkylene)_{b}-, -(alkylene-O)_{b}—, cycloalkylene, or heterocyclylene, where each ring is optionally substituted with one or two alkyl;
Z³ is a bond, alkylene, —C(O)NR—, —NR′(CO)—, —O—, —NR″—, cycloalkylene, phenylene, monocyclic heteroarylene, heterocyclylene, fused heterocyclylene, or spiro heterocyclylene, where each ring is optionally substituted with one or two alkyl;
Z⁴ is a bond, -(alkylene-NR″)—, —O—, —NR″—, cycloalkylene, phenylene, monocyclic heteroarylene, heterocyclylene, fused heterocyclylene, or spiro heterocyclylene, where each ring is optionally substituted with one or two alkyl;
Z is a bond; and
Z⁶ is —S(O)₂—; and
wherein each alkylene is optionally substituted with one, two, or three deuterium.
A116. In embodiment A116, the compound of any one of embodiments A1 to A96, or a pharmaceutically acceptable salt thereof, is wherein:
X¹, X², X³, and X⁴, and Z¹ are each a bond;
Z² is a bond, alkylene, cycloalkylene, or heterocyclylene, where each ring is optionally substituted with one or two substituents independently selected from alkyl, alkoxy, halo, haloalkyl, and haloalkoxy;
Z³ is a bond, alkylene, —C(O)NR—, —NR′(CO)—, —O—, —NR″—, cycloalkylene, phenylene, monocyclic heteroarylene, heterocyclylene, bicyclic heterocyclylene, bridged heterocyclylene, fused heterocyclylene, or spiro heterocyclylene, where each ring is optionally substituted with one or two substituents independently selected from alkyl, alkoxy, halo, haloalkyl, and haloalkoxy;
Z⁴ is a bond, alkylene, —O—, cycloalkylene, phenylene, monocyclic heteroarylene, heterocyclylene, fused heterocyclylene, or spiro heterocyclylene, where each ring is optionally substituted with one or two substituents independently selected from alkyl, alkoxy, halo, haloalkyl, and haloalkoxy;
Z⁵ is phenylene, monocyclic heteroarylene, or heterocycylene, where each ring is optionally substituted with one or two substituents independently selected from alkyl, alkoxy, halo, haloalkyl, and haloalkoxy; and
Z⁶ is —S(O)₂—; and
wherein each alkylene is optionally substituted with one, two, or three deuterium.
A17. In embodiment A117, the compound of any one of embodiments A1 to A96 and A16, or a pharmaceutically acceptable salt thereof, is wherein:
X¹, X², X³, and X⁴, Z¹, and Z² are each a bond;
Z³ is cycloalkylene, phenylene, monocyclic heteroarylene, heterocyclylene, bicyclic heterocyclylene, bridged heterocyclylene, fused heterocyclylene, or spiro heterocyclylene, where each ring is optionally substituted with one or two substituents independently selected from alkyl, alkoxy, halo, haloalkyl, and haloalkoxy;
Z⁴ is a bond, alkylene, —O—, cycloalkylene, phenylene, monocyclic heteroarylene, heterocyclylene, fused heterocyclylene, or spiro heterocyclylene, where each ring is optionally substituted with one or two substituents independently selected from alkyl, alkoxy, halo, haloalkyl, and haloalkoxy;
Z⁵ is phenylene, monocyclic heteroarylene, or heterocycylene, where each ring is optionally substituted with one or two substituents independently selected from alkyl, alkoxy, halo, haloalkyl, and haloalkoxy; and
Z⁶ is —S(O)₂—; and
wherein alkylene is optionally substituted with one or two deuterium.
A118. In embodiment A118, the compound of any one of embodiments A1 to A96, A16, and A17, or a pharmaceutically acceptable salt thereof, is wherein: X¹, X², X³, and X⁴, Z¹, and Z² are each a bond;
Z³ is heterocyclylene, bicyclic heterocyclylene, bridged heterocyclylene, fused heterocyclylene, or spiro heterocyclylene, where each ring is optionally substituted with one or two substituents independently selected from alkyl, alkoxy, halo, haloalkyl, and haloalkoxy;
Z⁴ is alkylene, —O—, monocyclic heteroarylene, heterocyclylene, fused heterocyclylene, or spiro heterocyclylene, where each ring is optionally substituted with one or two substituents independently selected from alkyl, alkoxy, halo, haloalkyl, and haloalkoxy;
Z⁵ is phenylene, monocyclic heteroarylene, or heterocycylene, where each ring is optionally substituted with one or two substituents independently selected from alkyl, alkoxy, halo, haloalkyl, and haloalkoxy; and
Z⁶ is —S(O)₂—; and
wherein alkylene is optionally substituted with one or two deuterium.
A119. In embodiment A119, the compound of any one of embodiments A1 to A96 and A16 to A18, or a pharmaceutically acceptable salt thereof, is wherein:
X¹, X², X³, and X⁴, Z¹, and Z² are each a bond;
Z³ is heterocyclylene, bridged heterocyclylene, or spiro heterocyclylene, where each ring is optionally substituted with one or two substituents independently selected from alkyl, alkoxy, halo, haloalkyl, and haloalkoxy;
Z⁴ is alkylene, —O—, or heterocyclylene, where each ring is optionally substituted with one or two substituents independently selected from alkyl, alkoxy, halo, haloalkyl, and haloalkoxy;
Z⁵ is phenylene, monocyclic heteroarylene (e.g., pyridindiyl), or heterocycylene, where each ring is optionally substituted with one or two substituents independently selected from alkyl, alkoxy, halo, haloalkyl, and haloalkoxy; and
Z⁶ is —S(O)₂—; and
wherein alkylene is optionally substituted with one or two deuterium.
A120. In embodiment A120, the compound of any one of embodiments A1 to A96 and A16 to A19, or a pharmaceutically acceptable salt thereof, is wherein: X¹, X², X³, and X⁴, Z¹, and Z² are each a bond;
Z³ is heterocyclylene, bridged heterocyclylene, or spiro heterocyclylene, where each ring is optionally substituted with one or two substituents independently selected from alkyl, alkoxy, halo, haloalkyl, and haloalkoxy;
Z⁴ is alkylene, —O—, or heterocyclylene, where each ring is optionally substituted with one or two substituents independently selected from alkyl, alkoxy, halo, haloalkyl, and haloalkoxy;
Z⁵ is phenylene optionally substituted with one or two substituents independently selected from alkyl, alkoxy, halo, haloalkyl, and haloalkoxy; and
Z⁶ is —S(O)₂—; and
wherein alkylene is optionally substituted with one or two deuterium.
A121. In embodiment A121, the compound of any one of embodiments A1 to A96, or a pharmaceutically acceptable salt thereof, is wherein:
X¹, X², X³, and X⁴, and Z¹ are each a bond;
Z² is cycloalkylene or heterocyclylene, where each ring is optionally substituted with one or two substituents independently selected from alkyl, alkoxy, halo, haloalkyl, and haloalkoxy;
Z³ is cycloalkylene, phenylene, monocyclic heteroarylene, heterocyclylene, bicyclic heterocyclylene, bridged heterocyclylene, fused heterocyclylene, or spiro heterocyclylene, where each ring is optionally substituted with one or two substituents independently selected from alkyl, alkoxy, halo, haloalkyl, and haloalkoxy;
Z⁴ is a bond, alkylene, or —O—;
Z⁵ is phenylene, monocyclic heteroarylene (e.g., pyridindiyl), or heterocycylene, where each ring is optionally substituted with one or two substituents independently selected from alkyl, alkoxy, halo, haloalkyl, and haloalkoxy; and
Z⁶ is —S(O)₂—.
A122. In embodiment A122, the compound of any one of embodiments A1 to A96 and A121, or a pharmaceutically acceptable salt thereof, is wherein:
X¹, X², X³, and X⁴, and Z¹ are each a bond;
Z² is heterocyclylene optionally substituted with one or two substituents independently selected from alkyl, alkoxy, halo, haloalkyl, and haloalkoxy;
Z³ is heterocyclylene optionally substituted with one or two substituents independently selected from alkyl, alkoxy, halo, haloalkyl, and haloalkoxy;
Z⁴ is a bond, alkylene, or —O—;
Z⁵ is phenylene optionally substituted with one or two substituents independently selected from alkyl, alkoxy, halo, haloalkyl, and haloalkoxy; and
Z⁶ is —S(O)₂—; and
wherein alkylene is optionally substituted with one or two deuterium.
A123. In embodiment A123, the compound of any one of embodiments A1 to A96, A121 and A122, or a pharmaceutically acceptable salt thereof, is wherein:
X¹, X², X³, and X⁴, and Z¹ are each a bond;
Z² is heterocyclylene, where each ring is optionally substituted with one or two substituents independently selected from alkyl, alkoxy, halo, haloalkyl, and haloalkoxy;
Z³ is a bond, alkylene, or —O—;
Z⁴ is heterocyclylene, bridged heterocyclylene, or spiro heterocyclylene, where each ring is optionally substituted with one or two substituents independently selected from alkyl, alkoxy, halo, haloalkyl, and haloalkoxy;
Z⁵ is phenylene optionally substituted with one or two substituents independently selected from alkyl, alkoxy, halo, haloalkyl, and haloalkoxy; and
Z⁶ is —S(O)₂—.
A123a. In embodiment A123, the compound of any one of embodiments A1 to A96, A121 and A122, or a pharmaceutically acceptable salt thereof, is wherein Z⁴ is heterocyclylene, or spiro heterocyclylene, where each ring is optionally substituted with one or two substituents independently selected from alkyl, alkoxy, halo, haloalkyl, and haloalkoxy.
A124. In embodiment A124, the compound of any one of embodiments A1 to A123a, or a pharmaceutically acceptable salt thereof, is wherein —Z⁵— is

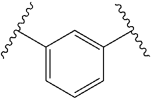 [SMILES: *c1cccc(*)c1]

(i.e., Z⁵ is phenylene where Z⁴ and Z⁶ are attached at meta position of the phenylene ring) optionally substituted with one or two substituents independently selected from alkyl, alkoxy, halo, haloalkyl, and haloalkoxy.
A125. In embodiment A125, the compound of any one of embodiments A1 to A123, or a pharmaceutically acceptable salt thereof, is wherein —Z⁵— is

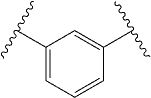 [SMILES: *c1cccc(*)c1]

optionally substituted with one or two substituents independently selected from methyl, methoxy, fluoro, chloro, difluoromethyl, trifluoromethyl, difluoromethoxy, and trifluoromethoxy.
A126. In embodiment A126, the compound of any one of embodiments A1 to A123, or a pharmaceutically acceptable salt thereof, is wherein —Z⁵— is

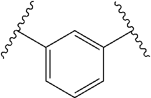 [SMILES: *c1cccc(*)c1]

optionally substituted with one or two substituents independently selected from methyl, fluoro, trifluoromethyl, and trifluoromethoxy.
A127. In embodiment A127, the compound of any one of embodiments A1 to A19 and A121, or a pharmaceutically acceptable salt thereof, is wherein Z is pyridin-2,4-diyl, pyridin-2,6-diyl, or pyridin-3,5-diyl optionally substituted with one substituent selected from alkyl, alkoxy, halo, haloalkyl, and haloalkoxy.
A128. In embodiment A128, the compound of any one of embodiments A1 to A19 and A121, or a pharmaceutically acceptable salt thereof, is wherein Z is pyridin-2,4-diyl, pyridin-2,6-diyl, or pyridin-3,5-diyl optionally substituted with one substituent selected from methyl, methoxy, fluoro, chloro, difluoromethyl, trifluoromethyl, difluoromethoxy, and trifluoromethoxy.
A129. In embodiment A129, the compound of any one of embodiments A1 to A128, or a pharmaceutically acceptable salt thereof, is wherein each alkylene of —Z¹-Z²—Z³—Z⁴-Z⁵—Z⁶—, by itself and when present, is methylene, ethylene, or propylene, each optionally substituted with one or two deuterium.
A130. In embodiment A130, the compound of any one of embodiments A1 to A128, or a pharmaceutically acceptable salt thereof, is wherein each alkylene of —Z¹-Z²—Z³—Z⁴-Z⁵—Z⁶—, by itself and when present, is methylene optionally substituted with one or two deuterium.
A131 In embodiment A131, the compound of any one of embodiments A1 to A130, or a pharmaceutically acceptable salt thereof, is wherein each alkylene of —Z¹-Z²—Z³—Z⁴-Z⁵—Z⁶—, as part of another group (e.g., —(O-alkylene)ₐ, -(alkylene-O)ₐ—, -(alkylene-NR″)—) and when present, is ethylene or propylene.
A132. In embodiment A132, the compound of any one of embodiments A1 to A131, or a pharmaceutically acceptable salt thereof, is wherein each alkylene of —Z¹-Z²—Z³—Z⁴-Z⁵—Z⁶—, as part of another group (e.g., —(O-alkylene)ₐ, -(alkylene-O)ₐ—, -(alkylene-NR″)—) and when present, is ethylene.
A133. In embodiment A133, the compound of any one of embodiments A1 to A132, or a pharmaceutically acceptable salt thereof, is wherein each R, R′ and R″ of —Z¹-Z²—Z³—Z⁴-Z⁵—Z⁶—, when present, is independently hydrogen or methyl.
A134. In embodiment A134, the compound of any one of embodiments A1 to A133, or a pharmaceutically acceptable salt thereof, is wherein each R, R′ and R″ of —Z¹-Z²—Z³—Z⁴-Z⁵—Z⁶—, when present, is hydrogen.
A135. In embodiment A135, the compound of any one of embodiments A1 to A133, or a pharmaceutically acceptable salt thereof, is wherein each R, R′ and R″ of —Z¹-Z²—Z³—Z⁴-Z⁵—Z⁶—, when present, is methyl.
A136. In embodiment A136, the compound of any one of embodiments A1 to A135, or a pharmaceutically acceptable salt thereof, is wherein each cycloalkylene of —Z¹-Z²—Z³—Z⁴-Z⁵—Z⁶—, when present, is independently selected from cyclopropylene, cyclobutylene, cyclopentylene, and cyclohexylene.
A137. In embodiment A137, the compound of any one of embodiments A1 to A136, or a pharmaceutically acceptable salt thereof, is wherein each cycloalkylene of —Z¹-Z²—Z³—Z⁴-Z⁵—Z⁶—, when present, is independently selected from 1,3-cyclopentylene, 1,3-cyclohexylene, and 1,4-cyclohexylene.
A138. In embodiment A138, the compound of any one of embodiments A1 to A137, or a pharmaceutically acceptable salt thereof, is wherein heteroarylene is monocyclic heteroarylene and each monocyclic heteroarylene of —Z¹-Z²—Z³—Z⁴-Z⁵—Z⁶—, when present, is independently selected from pyridindiyl and pyrimidinediyl unless stated otherwise in any of the embodiment above.
A139. In embodiment A139, the compound of any one of embodiments A1 to A138, or a pharmaceutically acceptable salt thereof, is wherein heteroarylene is monocyclic heteroarylene and each monocyclic heteroarylene of —Z¹-Z²—Z³—Z⁴-Z⁵—Z⁶—, when present, is independently selected from pyridin-2,4-diyl, pyridin-2,6-diyl, and pyridin-3,5-diyl, unless stated otherwise in any of the embodiment above.
A140. In embodiment A140, the compound of any one of embodiments A1 to A139, or a pharmaceutically acceptable salt thereof, is wherein phenylene of —Z¹-Z²—Z³—Z⁴-Z⁵—Z⁶—, when present, is independently selected from 1,3-phenylene and 1,4-phenylene unless stated otherwise in any of the embodiment above.
A141. In embodiment A141, the compound of any one of embodiments A1 to A140, or a pharmaceutically acceptable salt thereof, is wherein heterocyclylene, bridged heterocyclylene, and spiro heterocyclylene, of —Z¹-Z²—Z³—Z⁴-Z⁵—Z⁶—, when present, are independently selected from

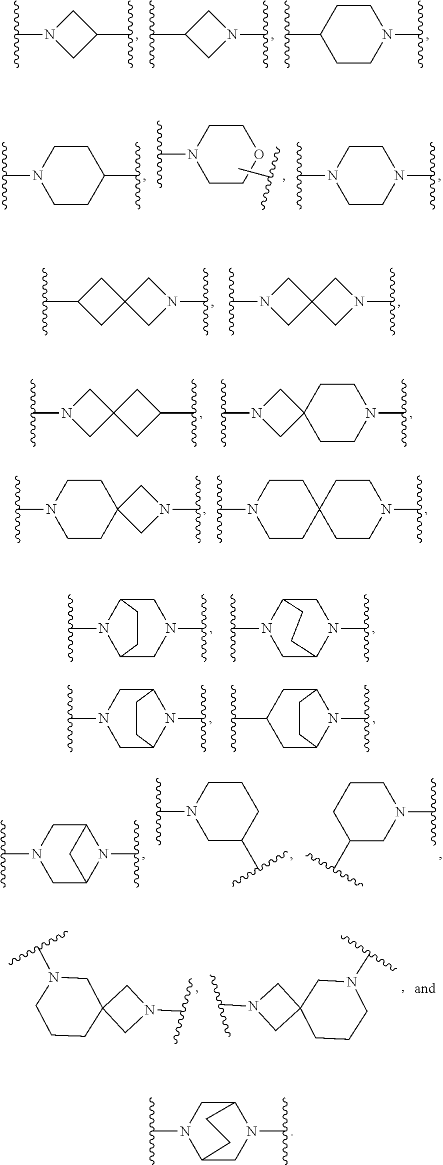

A142. In embodiment A142, the compound of any one of embodiments A1 to A96, or a pharmaceutically acceptable salt thereof, is wherein —X¹-L-, —X²-L-, —X³-L- and —X⁴-L- are independently selected from:

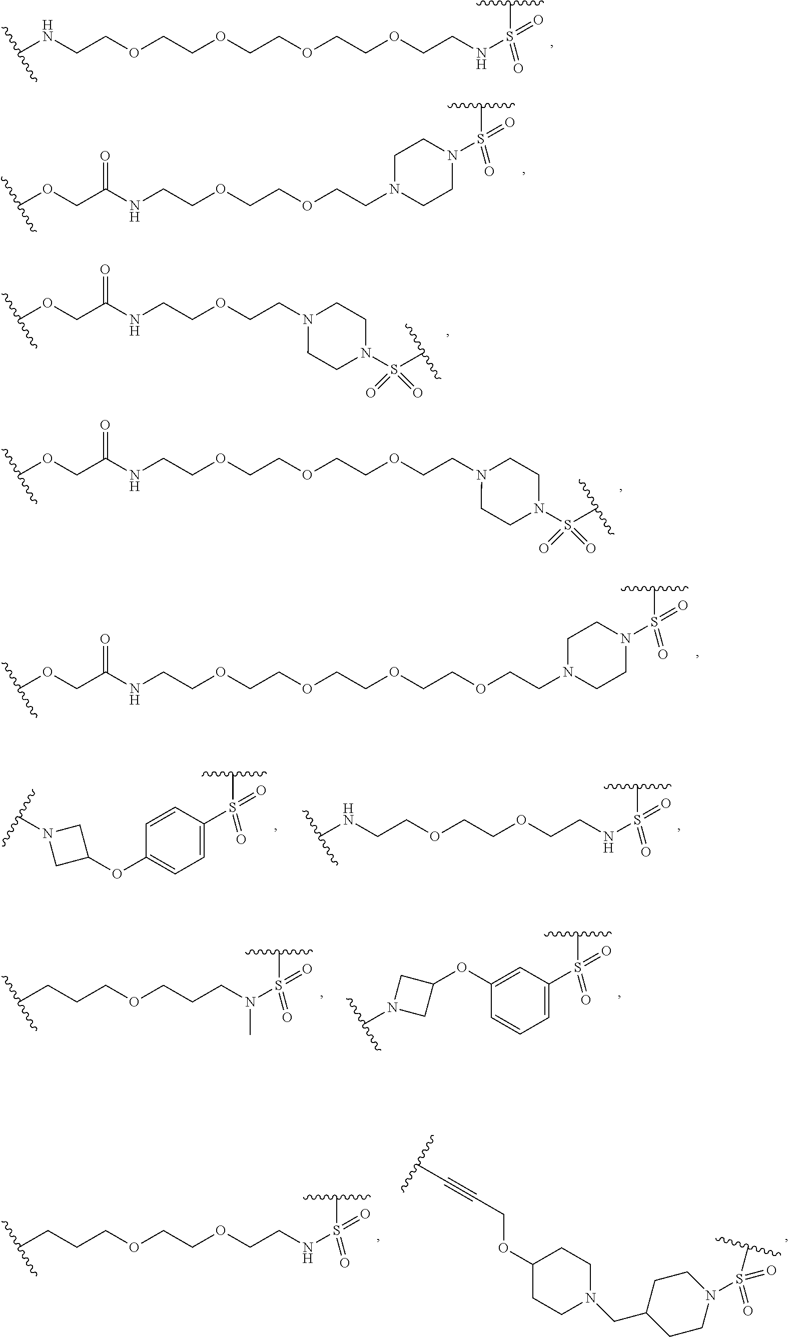

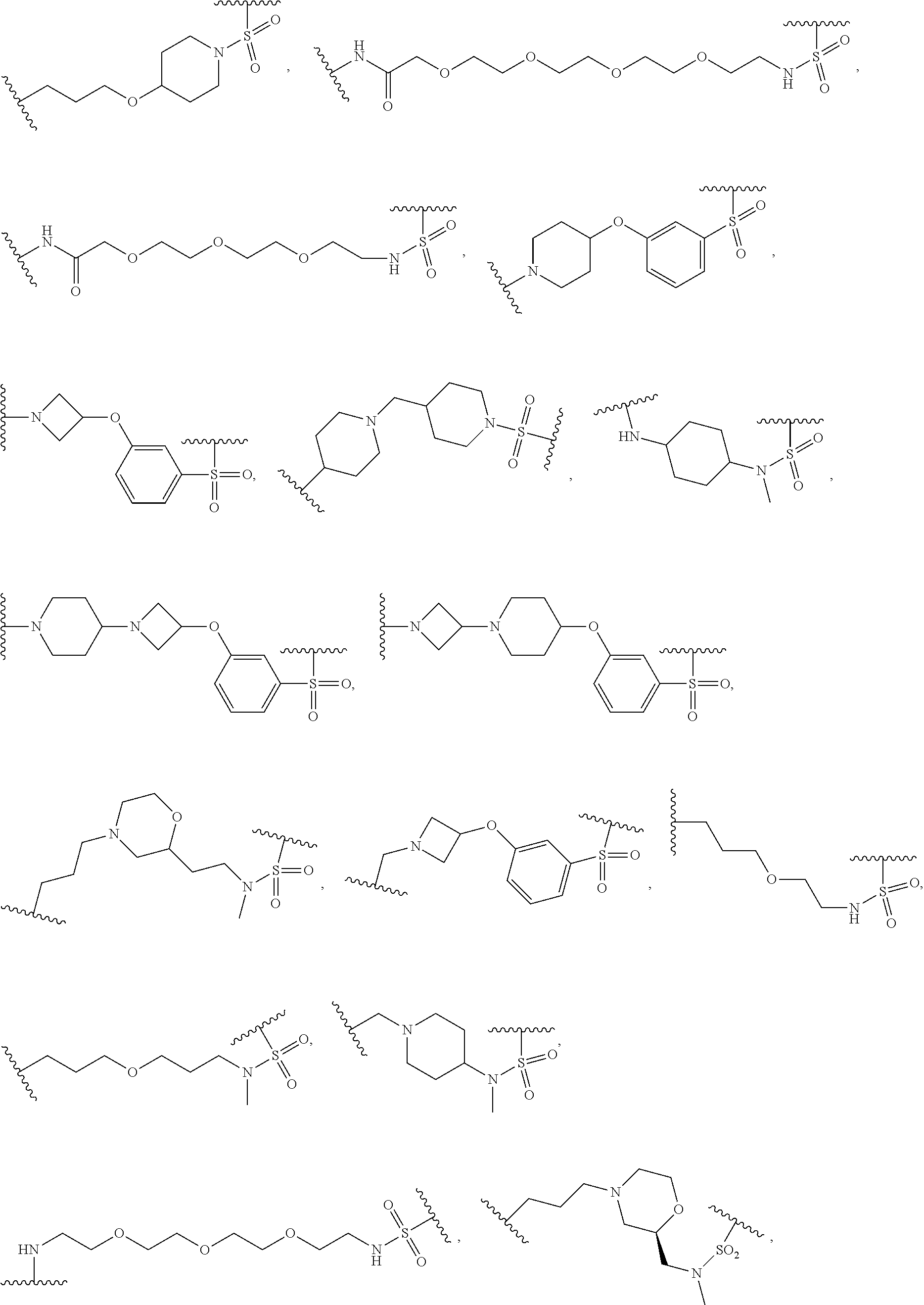

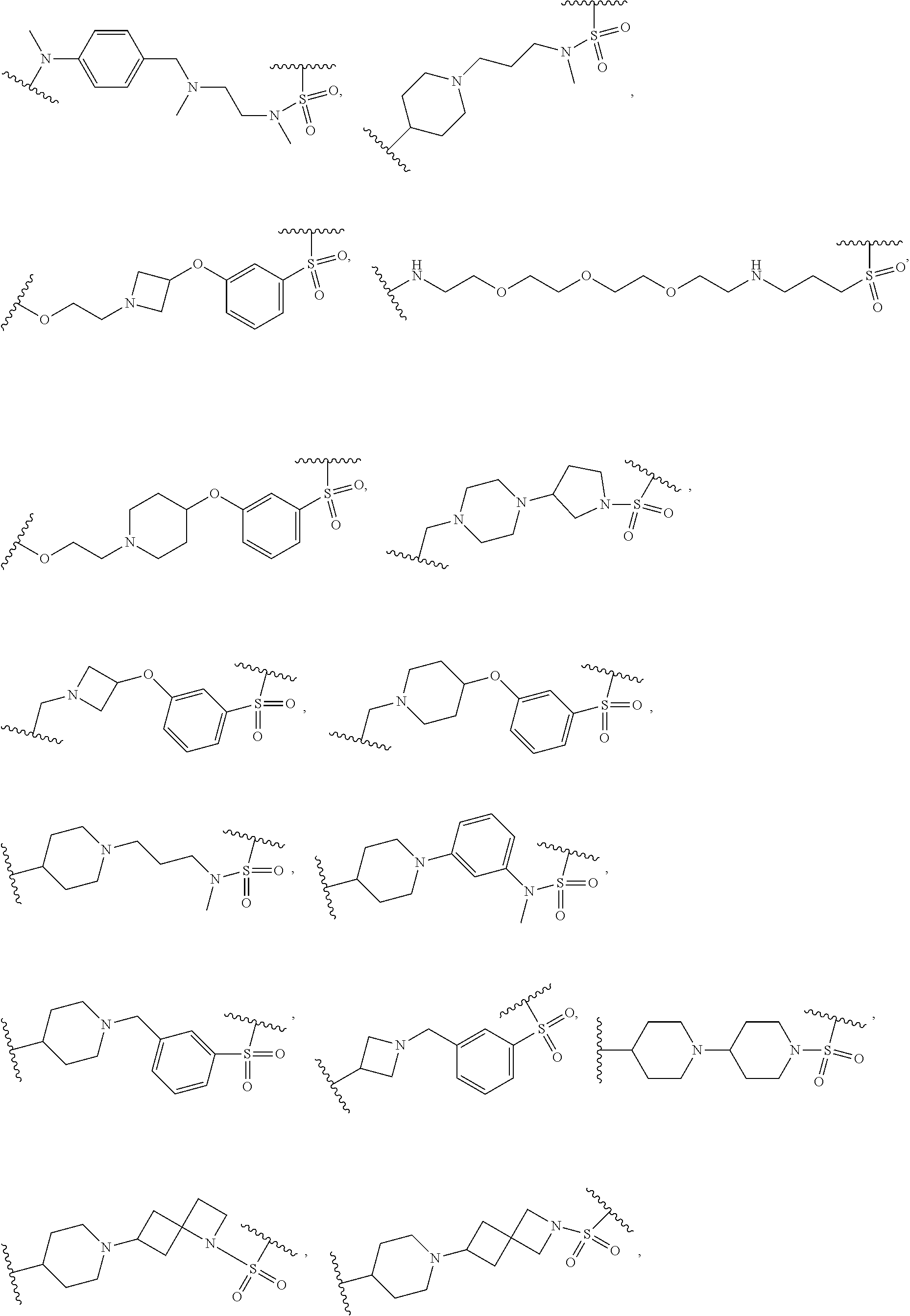

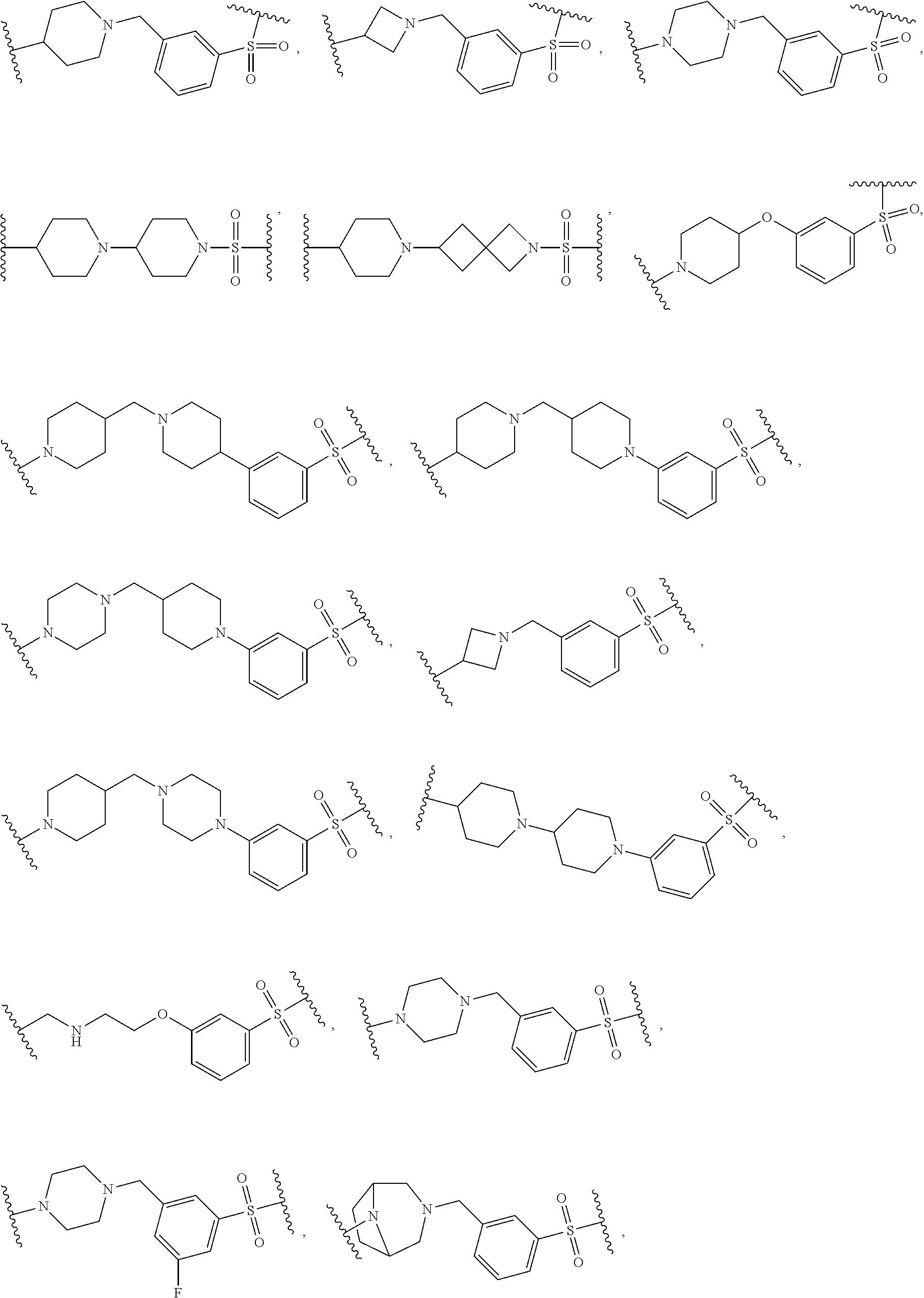

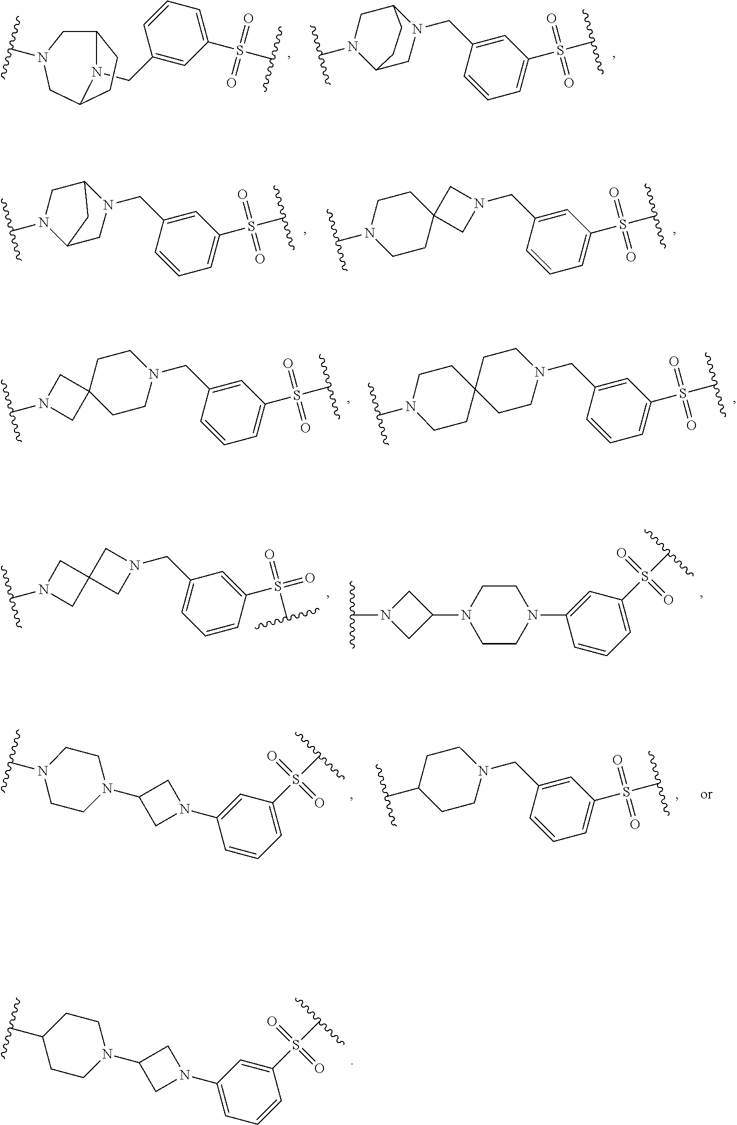

A143. In embodiment A143, the compound of any one of embodiments A1 to A96, or a pharmaceutically acceptable salt thereof, is wherein —X¹-L-, —X²-L-, —X³-L-, and —X⁴-L- are independently selected from:

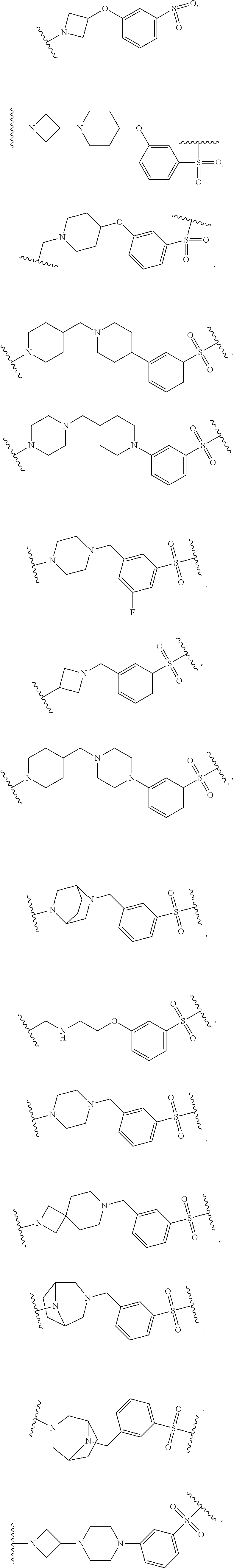

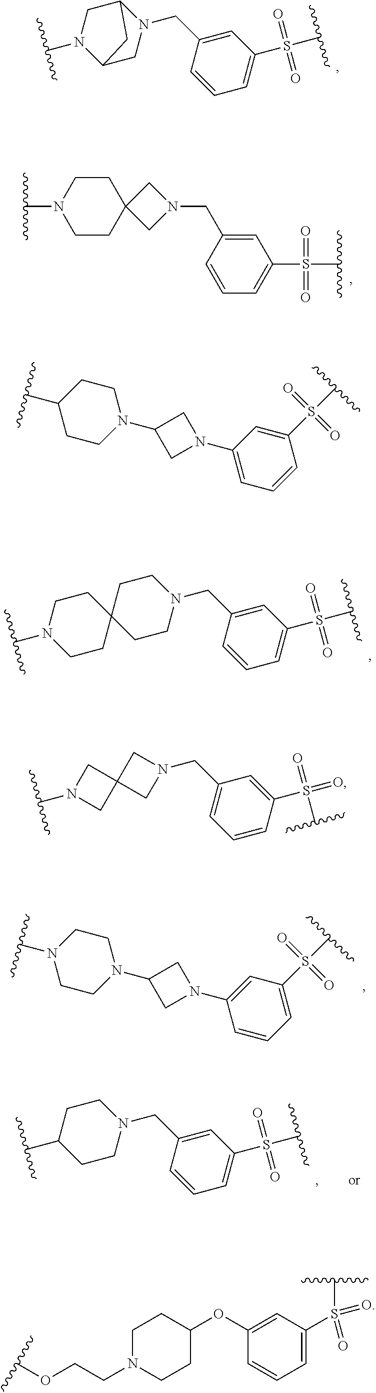

A144. In embodiment A144, the compound of any one of embodiments A1 to A143, or a pharmaceutically acceptable salt thereof, is wherein the E3 ligase ligand is:

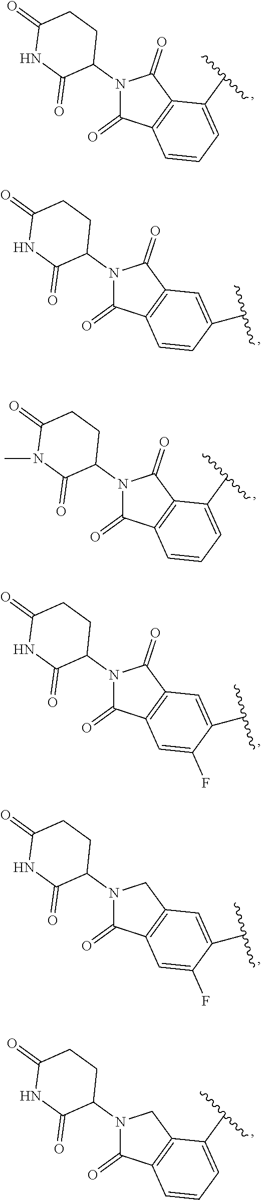 [SMILES: *c1cccc2c1C(=O)N(C1CCC(=O)NC1=O)C2=O ; *c1ccc2c(c1)C(=O)N(C1CCC(=O)NC1=O)C2=O ; *c1cccc2c1C(=O)N(C1CCC(=O)N(C)C1=O)C2=O ; *c1cc2c(cc1F)C(=O)N(C1CCC(=O)NC1=O)C2=O ; *c1cc2c(cc1F)C(=O)N(C1CCC(=O)NC1=O)C2 ; *c1cccc2c1CN(C1CCC(=O)NC1=O)C2=O]

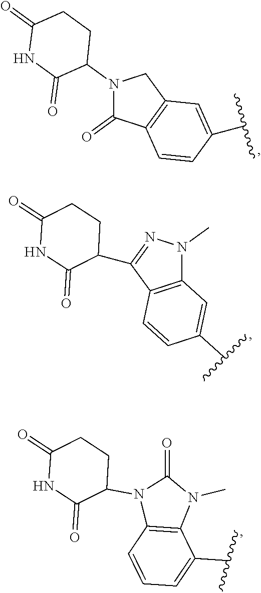 [SMILES: *c1ccc2c(c1)CN(C1CCC(=O)NC1=O)C2=O ; *c1ccc2c(C3CCC(=O)NC3=O)nn(C)c2c1 ; *c1cccc2c1n(C)c(=O)n2C1CCC(=O)NC1=O]

A144a. In embodiment A144a, the compound of any one of embodiments A1 to A144 is wherein R^{x} is hydrogen.
A145. In embodiment A144, the compound is selected from Compound Table I.
A146. In embodiment A146, provided is a pharmaceutical composition comprising a compound of any one of embodiments A1 to A145, or a pharmaceutically acceptable salt thereof, and a pharmaceutically acceptable excipient.
A147. In embodiment A147, provided is a method of degrading CDK2 in a cell which method comprises contacting the cell with a compound of any one of embodiments 1A1 to AA145, or a pharmaceutically acceptable salt thereof, or a pharmaceutical composition of embodiment A146.
A148. In embodiment A148, provided is a method of treating a disease mediated by CDK2 in a patient which method comprises administering to the patient in recognized need thereof, a therapeutically effective amount of a pharmaceutical composition comprising a compound of any one of embodiments A1 to A145, or a pharmaceutically acceptable salt thereof, and a pharmaceutically acceptable excipient.
A149. In embodiment A149, provided is a method of treating cancer in a patient which method comprises administering to the patient in need thereof, a therapeutically effective amount a compound of any one of embodiments A1 to A145, or a pharmaceutically acceptable salt thereof, in a pharmaceutical composition comprising the compound of any one of embodiments A1 to A145 and a pharmaceutically acceptable excipient.
A150. In embodiment A150, the method of embodiment A149 is wherein the compound of any one of embodiments A1 to A145 or a pharmaceutically acceptable salt thereof, is administered in combination with at least one other anticancer agent.
A151. In embodiment A151, the method of embodiments A149 or A150 is wherein the cancer is lung cancer, skin cancer, bladder cancer, breast cancer, cervical cancer, colorectal cancer, cancer of the small intestine, colon cancer, rectal cancer, cancer of the anus, endometrial cancer, gastric cancer, head and neck cancer, liver cancer, ovarian cancer, prostate cancer, testicular cancer, uterine cancer, esophageal cancer, gall bladder cancer, pancreatic cancer, stomach cancer, thyroid cancer, or parathyroid cancer.

It is understood that the embodiments and subembodiments set forth above include all combination of embodiments and subembodiments listed therein.

TABLE IA

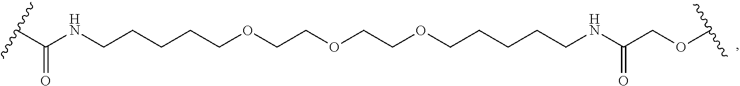 [SMILES: *OCC(=O)NCCCCCOCCOCCOCCCCCNC(*)=O]

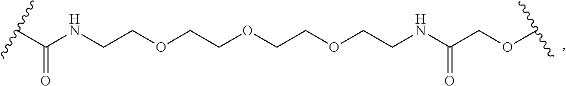 [SMILES: *OCC(=O)NCCOCCOCCOCCNC(*)=O]

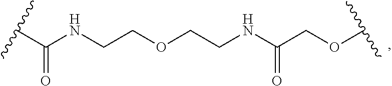 [SMILES: *OCC(=O)NCCOCCNC(*)=O]

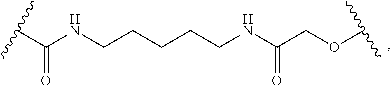 [SMILES: *OCC(=O)NCCCCCNC(*)=O]

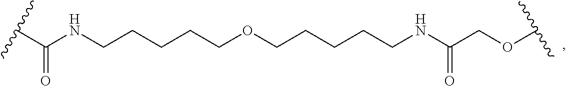 [SMILES: *OCC(=O)NCCCCCOCCCCCNC(*)=O]

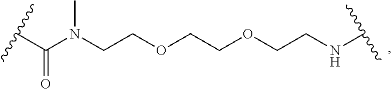 [SMILES: *NCCOCCOCCN(C)C(*)=O]

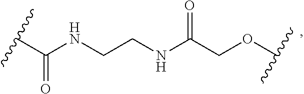 [SMILES: *OCC(=O)NCCNC(*)=O]

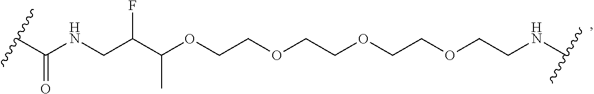 [SMILES: *NCCOCCOCCOCCOC(C)C(F)CNC(*)=O]

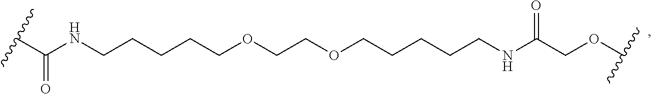 [SMILES: *OCC(=O)NCCCCCOCCOCCCCCNC(*)=O]

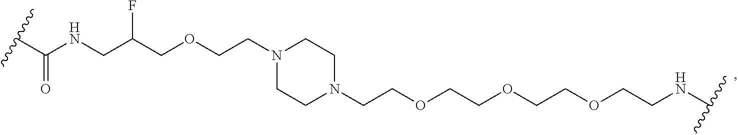 [SMILES: *NCCOCCOCCOCCN1CCN(CCOCC(F)CNC(*)=O)CC1]

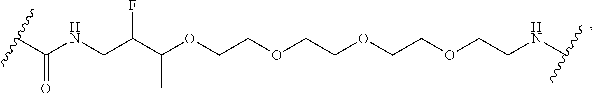 [SMILES: *NCCOCCOCCOCCOC(C)C(F)CNC(*)=O]

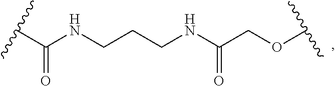 [SMILES: *OCC(=O)NCCCNC(*)=O]

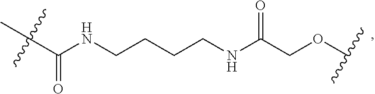 [SMILES: *OCC(=O)NCCCCNC(*)=O]

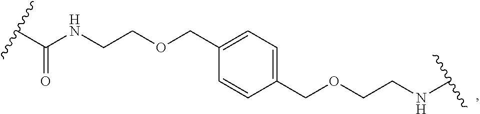 [SMILES: *NCCOCc1ccc(COCCNC(*)=O)cc1]

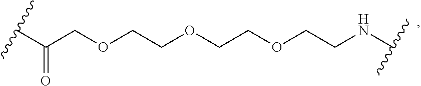 [SMILES: *NCCOCCOCCOCC(*)=O]

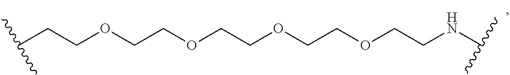 [SMILES: *CCOCCOCCOCCOCCN*]

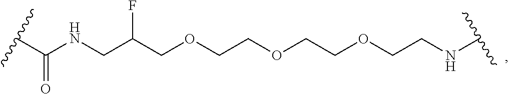 [SMILES: *NCCOCCOCCOCC(F)CNC(*)=O]

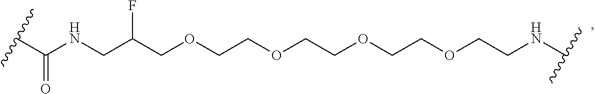 [SMILES: *NCCOCCOCCOCCOCC(F)CNC(*)=O]

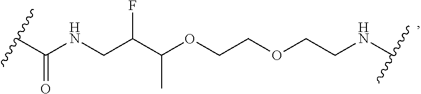 [SMILES: *NCCOCCOC(C)C(F)CNC(*)=O]

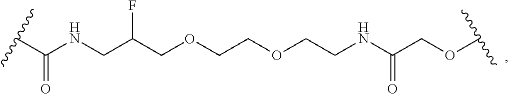 [SMILES: *OCC(=O)NCCOCCOCC(F)CNC(*)=O]

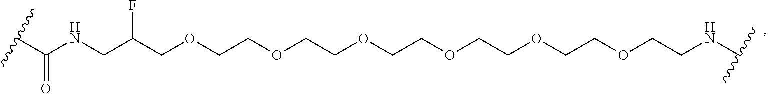 [SMILES: *NCCOCCOCCOCCOCCOCCOCC(F)CNC(*)=O]

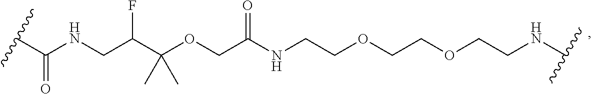 [SMILES: *NCCOCCOCCNC(=O)COC(C)(C)C(F)CNC(*)=O]

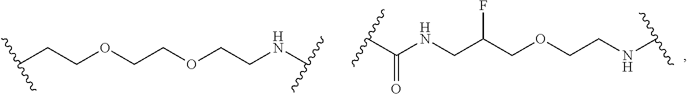 [SMILES: *CCOCCOCCN* ; *NCCOCC(F)CNC(*)=O]

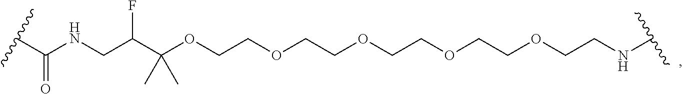 [SMILES: *NCCOCCOCCOCCOCCOC(C)(C)C(F)CNC(*)=O]

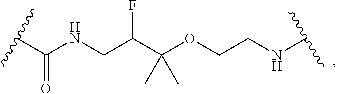 [SMILES: *NCCOC(C)(C)C(F)CNC(*)=O]

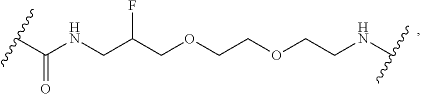 [SMILES: *NCCOCCOCC(F)CNC(*)=O]

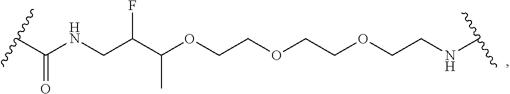 [SMILES: *NCCOCCOCCOC(C)C(F)CNC(*)=O]

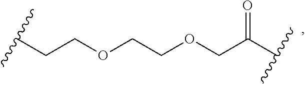 [SMILES: *CCOCCOCC(*)=O]

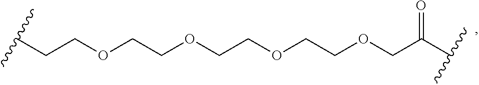 [SMILES: *CCOCCOCCOCCOCC(*)=O]

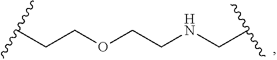 [SMILES: *CCOCCNC*]

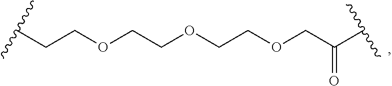 [SMILES: *CCOCCOCCOCC(*)=O]

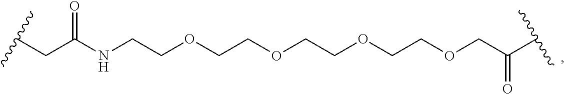 [SMILES: *CC(=O)NCCOCCOCCOCCOCC(*)=O]

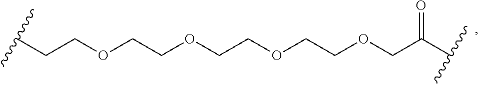 [SMILES: *CCOCCOCCOCCOCC(*)=O]

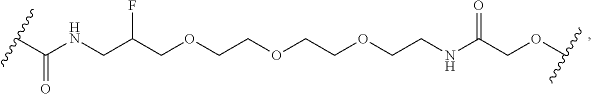 [SMILES: *OCC(=O)NCCOCCOCCOCC(F)CNC(*)=O]

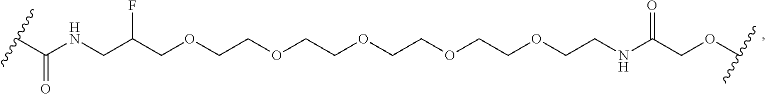 [SMILES: *OCC(=O)NCCOCCOCCOCCOCCOCC(F)CNC(*)=O]

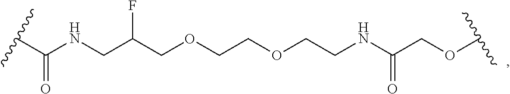 [SMILES: *OCC(=O)NCCOCCOCC(F)CNC(*)=O]

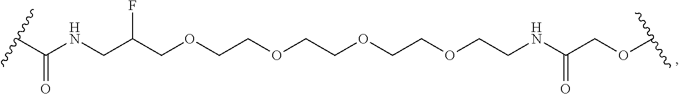 [SMILES: *OCC(=O)NCCOCCOCCOCCOCC(F)CNC(*)=O]

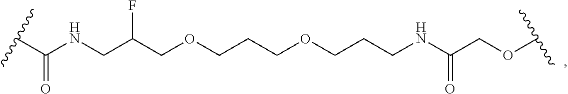 [SMILES: *OCC(=O)NCCCOCCCOCC(F)CNC(*)=O]

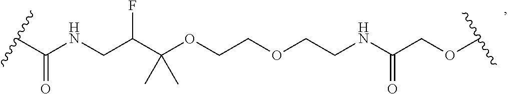 [SMILES: *OCC(=O)NCCOCCOC(C)(C)C(F)CNC(*)=O]

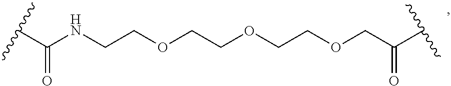 [SMILES: *C(=O)COCCOCCOCCNC(*)=O]

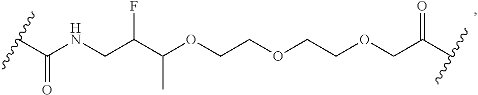 [SMILES: *C(=O)COCCOCCOC(C)C(F)CNC(*)=O]

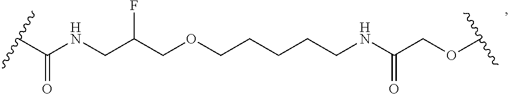 [SMILES: *OCC(=O)NCCCCCOCC(F)CNC(*)=O]

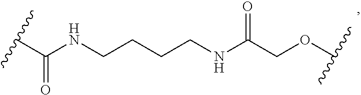 [SMILES: *OCC(=O)NCCCCNC(*)=O]

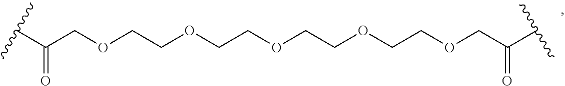 [SMILES: *C(=O)COCCOCCOCCOCCOCC(*)=O]

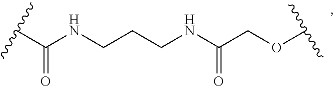 [SMILES: *OCC(=O)NCCCNC(*)=O]

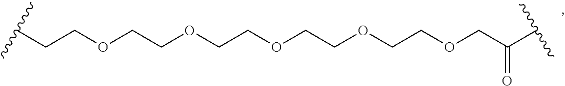 [SMILES: *CCOCCOCCOCCOCCOCC(*)=O]

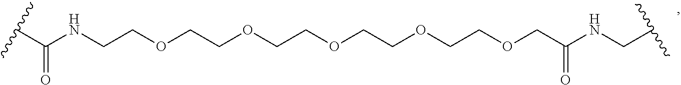 [SMILES: *CNC(=O)COCCOCCOCCOCCOCCNC(*)=O]

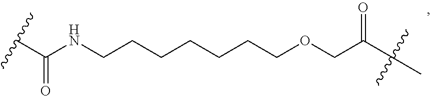 [SMILES: *C(=O)COCCCCCCCNC(*)=O]

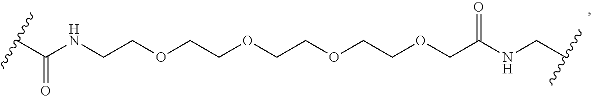 [SMILES: *CNC(=O)COCCOCCOCCOCCNC(*)=O]

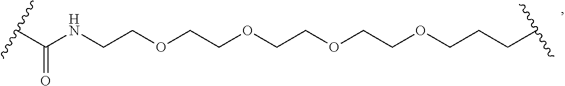 [SMILES: *CCCOCCOCCOCCOCCNC(*)=O]

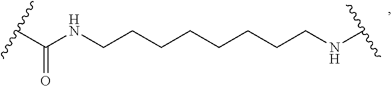 [SMILES: *NCCCCCCCCNC(*)=O]

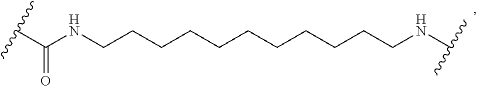 [SMILES: *NCCCCCCCCCCCNC(*)=O]

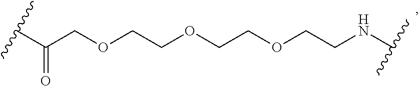 [SMILES: *NCCOCCOCCOCC(*)=O]

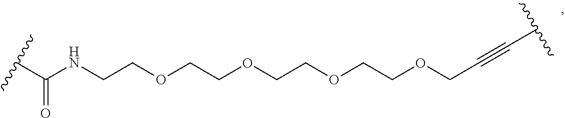 [SMILES: *C#CCOCCOCCOCCOCCNC(*)=O]

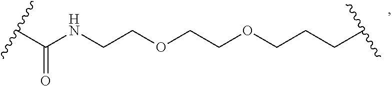 [SMILES: *CCCOCCOCCNC(*)=O]

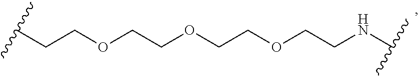 [SMILES: *CCOCCOCCOCCN*]

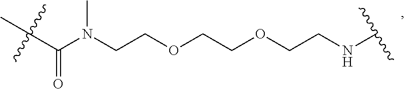 [SMILES: *NCCOCCOCCN(C)C(*)=O]

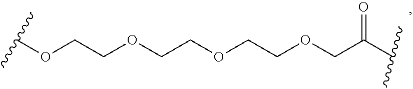 [SMILES: *OCCOCCOCCOCC(*)=O]

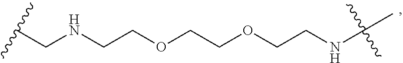 [SMILES: *CNCCOCCOCCN*]

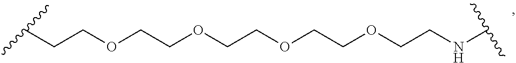 [SMILES: *CCOCCOCCOCCOCCN*]

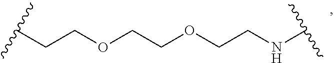 [SMILES: *CCOCCOCCN*]

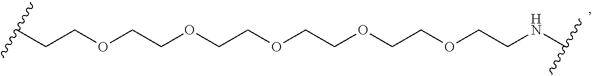 [SMILES: *CCOCCOCCOCCOCCOCCN*]

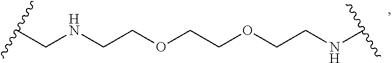 [SMILES: *CNCCOCCOCCN*]

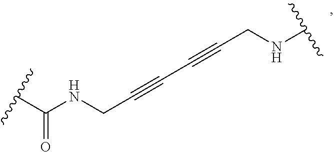 [SMILES: *NCC#CC#CCNC(*)=O]

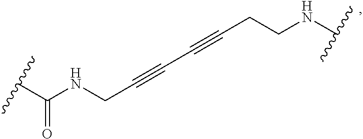 [SMILES: *NCCC#CC#CCNC(*)=O]

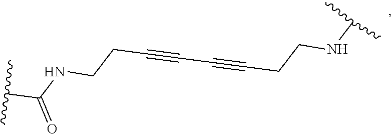 [SMILES: *NCCC#CC#CCCNC(*)=O]

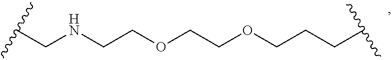 [SMILES: *CCCOCCOCCNC*]

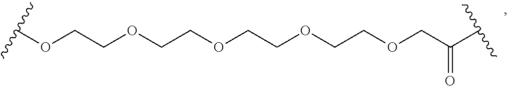 [SMILES: *OCCOCCOCCOCCOCC(*)=O]

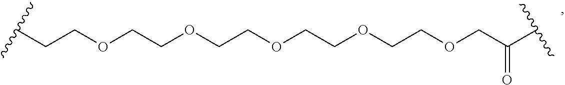 [SMILES: *CCOCCOCCOCCOCCOCC(*)=O]

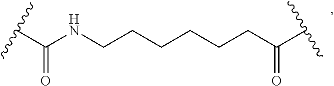 [SMILES: *C(=O)CCCCCCNC(*)=O]

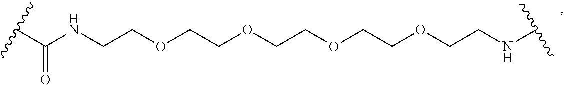 [SMILES: *NCCOCCOCCOCCOCCNC(*)=O]

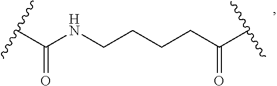 [SMILES: *C(=O)CCCCNC(*)=O]

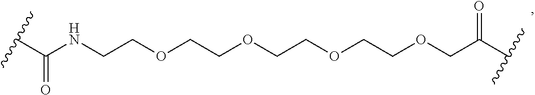 [SMILES: *C(=O)COCCOCCOCCOCCNC(*)=O]

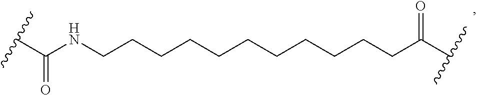 [SMILES: *C(=O)CCCCCCCCCCCNC(*)=O]

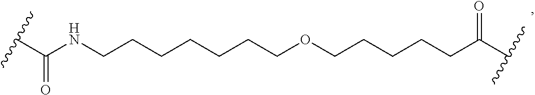 [SMILES: *C(=O)CCCCCOCCCCCCCNC(*)=O]

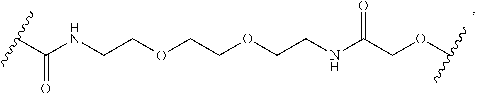 [SMILES: *OCC(=O)NCCOCCOCCNC(*)=O]

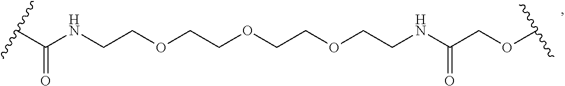 [SMILES: *OCC(=O)NCCOCCOCCOCCNC(*)=O]

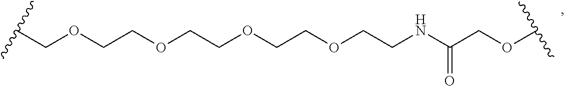 [SMILES: *COCCOCCOCCOCCNC(=O)CO*]

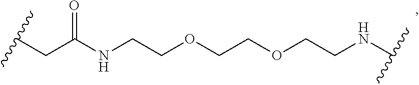 [SMILES: *CC(=O)NCCOCCOCCN*]

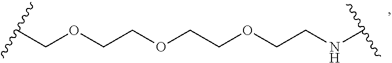 [SMILES: *COCCOCCOCCN*]

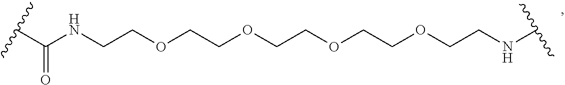 [SMILES: *NCCOCCOCCOCCOCCNC(*)=O]

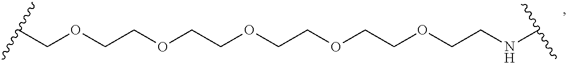 [SMILES: *COCCOCCOCCOCCOCCN*]

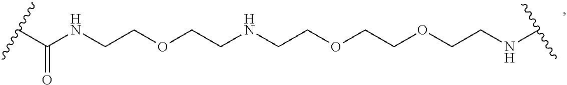 [SMILES: *NCCOCCOCCNCCOCCNC(*)=O]

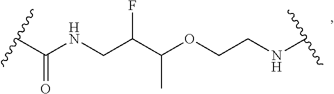 [SMILES: *NCCOC(C)C(F)CNC(*)=O]

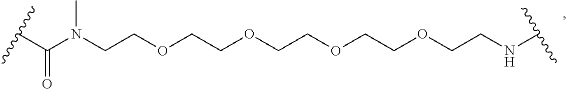 [SMILES: *NCCOCCOCCOCCOCCN(C)C(*)=O]

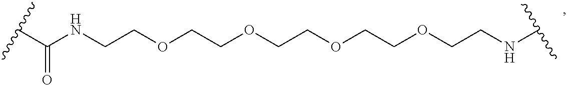 [SMILES: *NCCOCCOCCOCCOCCNC(*)=O]

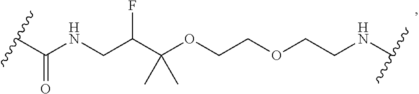 [SMILES: *NCCOCCOC(C)(C)C(F)CNC(*)=O]

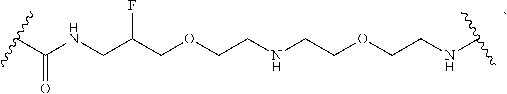 [SMILES: *NCCOCCNCCOCC(F)CNC(*)=O]

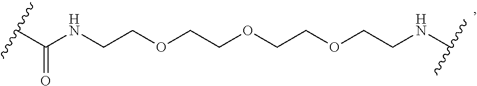 [SMILES: *NCCOCCOCCOCCNC(*)=O]

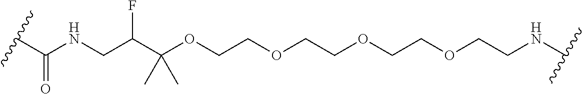 [SMILES: *NCCOCCOCCOCCOC(C)(C)C(F)CNC(*)=O]

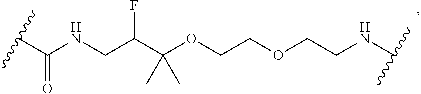 [SMILES: *NCCOCCOC(C)(C)C(F)CNC(*)=O]

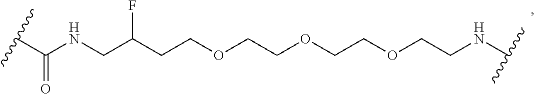 [SMILES: *NCCOCCOCCOCCC(F)CNC(*)=O]

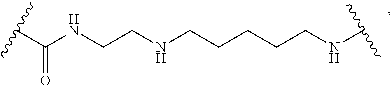 [SMILES: *NCCCCCNCCNC(*)=O]

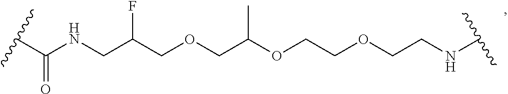 [SMILES: *NCCOCCOC(C)COCC(F)CNC(*)=O]

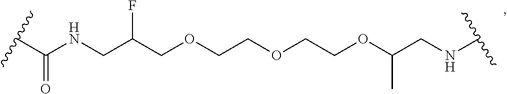 [SMILES: *NCC(C)OCCOCCOCC(F)CNC(*)=O]

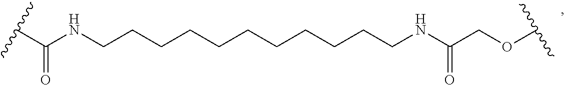 [SMILES: *OCC(=O)NCCCCCCCCCCCNC(*)=O]

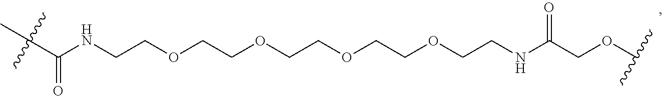 [SMILES: *OCC(=O)NCCOCCOCCOCCOCCNC(*)=O]

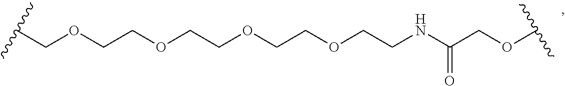 [SMILES: *COCCOCCOCCOCCNC(=O)CO*]

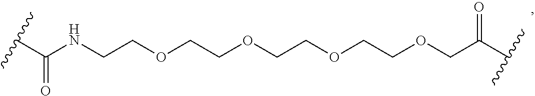 [SMILES: *C(=O)COCCOCCOCCOCCNC(*)=O]

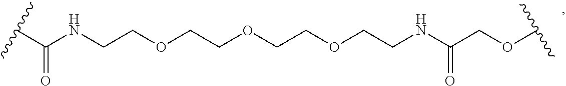 [SMILES: *OCC(=O)NCCOCCOCCOCCNC(*)=O]

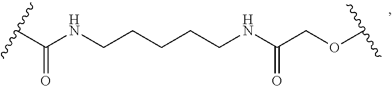 [SMILES: *OCC(=O)NCCCCCNC(*)=O]

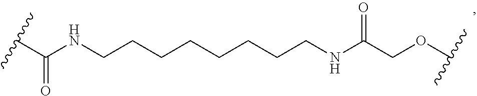 [SMILES: *OCC(=O)NCCCCCCCCNC(*)=O]

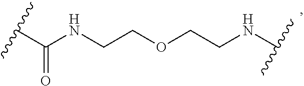 [SMILES: *NCCOCCNC(*)=O]

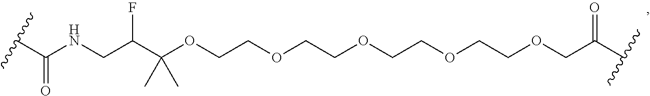 [SMILES: *C(=O)COCCOCCOCCOCCOC(C)(C)C(F)CNC(*)=O]

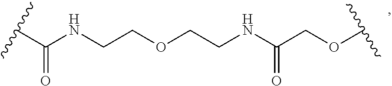 [SMILES: *OCC(=O)NCCOCCNC(*)=O]

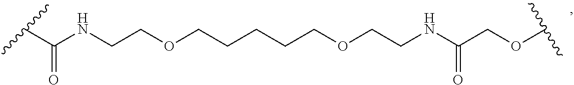 [SMILES: *OCC(=O)NCCOCCCCCOCCNC(*)=O]

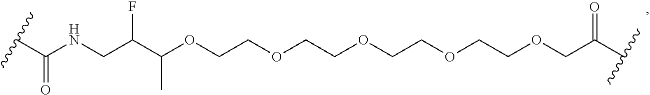 [SMILES: *C(=O)COCCOCCOCCOCCOC(C)C(F)CNC(*)=O]

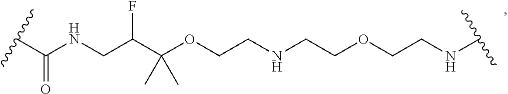 [SMILES: *NCCOCCNCCOC(C)(C)C(F)CNC(*)=O]

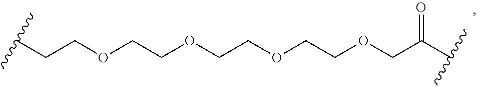 [SMILES: *CCOCCOCCOCCOCC(*)=O]

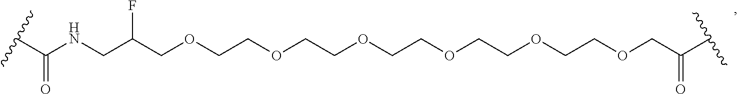 [SMILES: *C(=O)COCCOCCOCCOCCOCCOCC(F)CNC(*)=O]

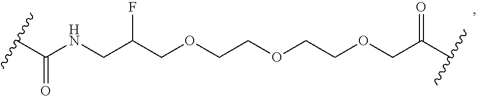 [SMILES: *C(=O)COCCOCCOCC(F)CNC(*)=O]

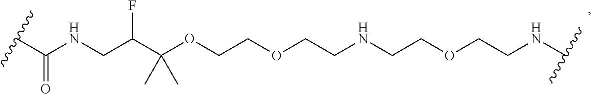 [SMILES: *NCCOCCNCCOCCOC(C)(C)C(F)CNC(*)=O]

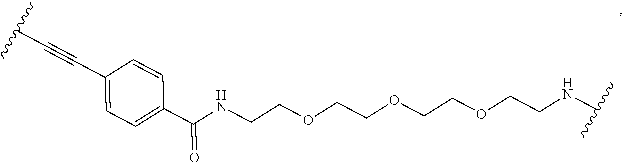 [SMILES: *C#Cc1ccc(C(=O)NCCOCCOCCOCCN*)cc1]

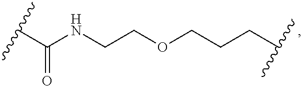 [SMILES: *CCCOCCNC(*)=O]

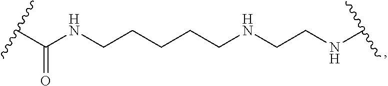 [SMILES: *NCCNCCCCCNC(*)=O]

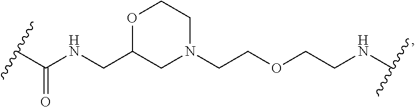 [SMILES: *NCCOCCN1CCOC(CNC(*)=O)C1]

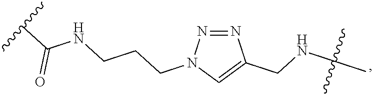 [SMILES: *NCc1cn(CCCNC(*)=O)nn1]

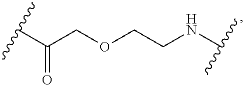 [SMILES: *NCCOCC(*)=O]

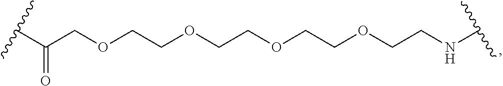 [SMILES: *NCCOCCOCCOCCOCC(*)=O]

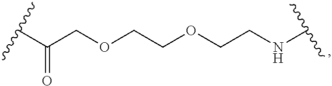 [SMILES: *NCCOCCOCC(*)=O]

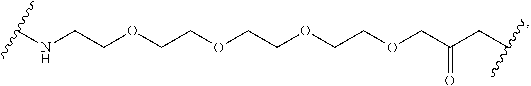 [SMILES: *CC(=O)COCCOCCOCCOCCN*]

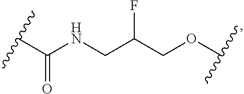 [SMILES: *OCC(F)CNC(*)=O]

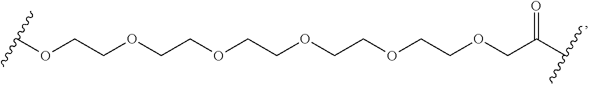 [SMILES: *OCCOCCOCCOCCOCCOCC(*)=O]

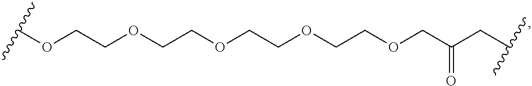 [SMILES: *CC(=O)COCCOCCOCCOCCO*]

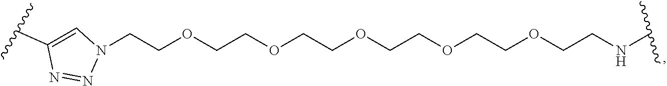 [SMILES: *NCCOCCOCCOCCOCCOCCn1cc(*)nn1]

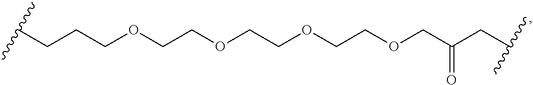 [SMILES: *CCCOCCOCCOCCOCC(=O)C*]

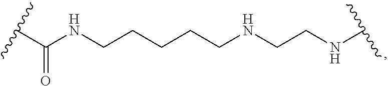 [SMILES: *NCCNCCCCCNC(*)=O]

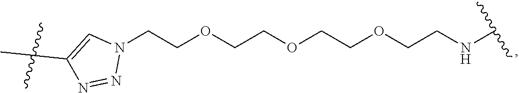 [SMILES: *NCCOCCOCCOCCn1cc(*)nn1]

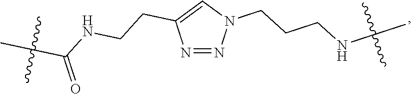 [SMILES: *NCCCn1cc(CCNC(*)=O)nn1]

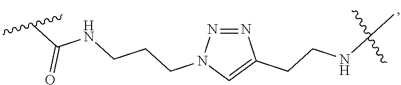 [SMILES: *NCCc1cn(CCCNC(*)=O)nn1]

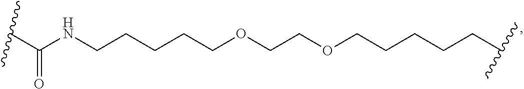 [SMILES: *CCCCCOCCOCCCCCNC(*)=O]

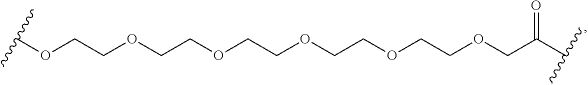 [SMILES: *OCCOCCOCCOCCOCCOCC(*)=O]

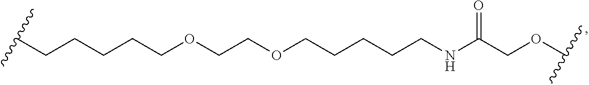 [SMILES: *CCCCCOCCOCCCCCNC(=O)CO*]

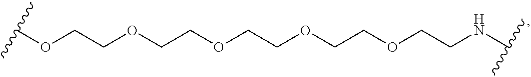 [SMILES: *NCCOCCOCCOCCOCCO*]

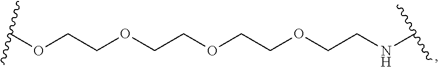 [SMILES: *NCCOCCOCCOCCO*]

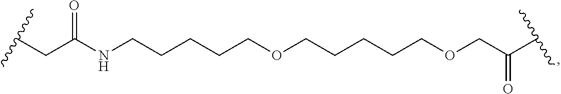 [SMILES: *CC(=O)NCCCCCOCCCCCOCC(*)=O]

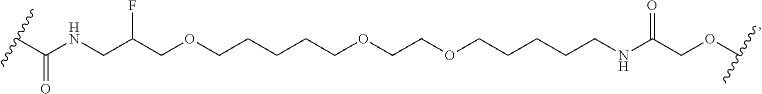 [SMILES: *OCC(=O)NCCCCCOCCOCCCCCOCC(F)CNC(*)=O]

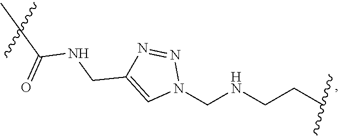 [SMILES: *CCNCn1cc(CNC(*)=O)nn1]

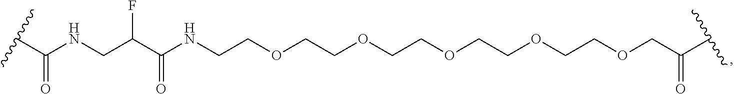 [SMILES: *C(=O)COCCOCCOCCOCCOCCNC(=O)C(F)CNC(*)=O]

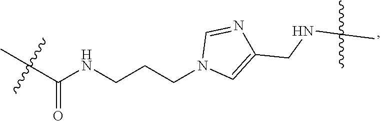 [SMILES: *NCc1cn(CCCNC(*)=O)cn1]

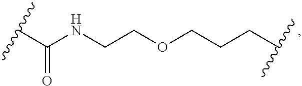 [SMILES: *CCCOCCNC(*)=O]

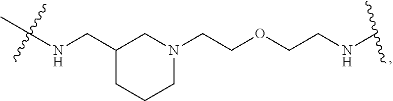 [SMILES: *NCCOCCN1CCCC(CN*)C1]

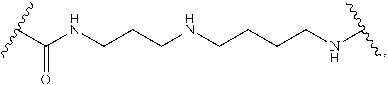 [SMILES: *NCCCCNCCCNC(*)=O]

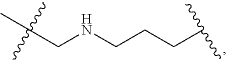 [SMILES: *CCCNC*]

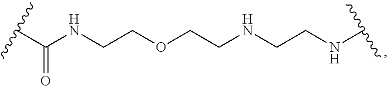 [SMILES: *NCCNCCOCCNC(*)=O]

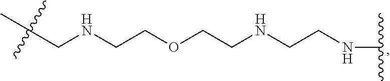 [SMILES: *CNCCOCCNCCN*]

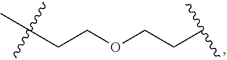 [SMILES: *CCOCC*]

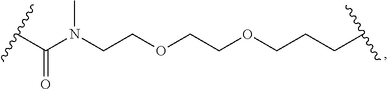 [SMILES: *CCCOCCOCCN(C)C(*)=O]

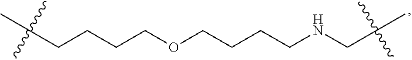 [SMILES: *CCCCOCCCCNC*]

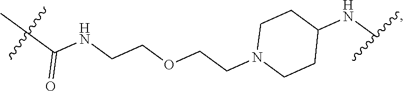 [SMILES: *NC1CCN(CCOCCNC(*)=O)CC1]

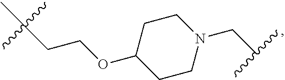 [SMILES: *CCOC1CCN(C*)CC1]

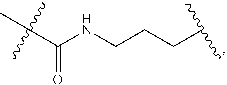 [SMILES: *CCCNC(*)=O]

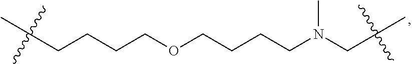 [SMILES: *CCCCOCCCCN(C)C*]

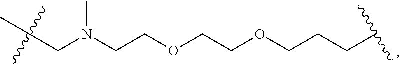 [SMILES: *CCCOCCOCCN(C)C*]

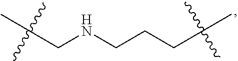 [SMILES: *CCCNC*]

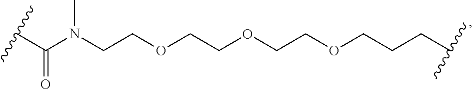 [SMILES: *CCCOCCOCCOCCN(C)C(*)=O]

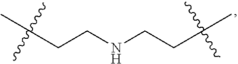 [SMILES: *CCNCC*]

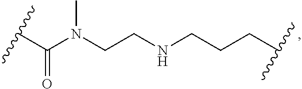 [SMILES: *CCCNCCN(C)C(*)=O]

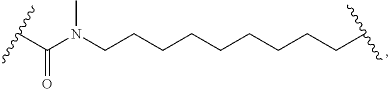 [SMILES: *CCCCCCCCCN(C)C(*)=O]

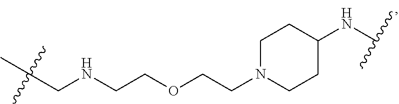 [SMILES: *CNCCOCCN1CCC(N*)CC1]

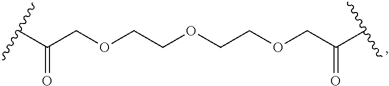 [SMILES: *C(=O)COCCOCCOCC(*)=O]

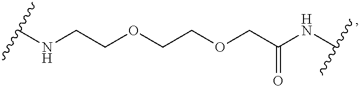 [SMILES: *NCCOCCOCC(=O)N*]

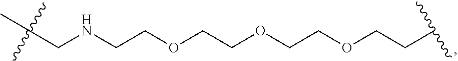 [SMILES: *CCOCCOCCOCCNC*]

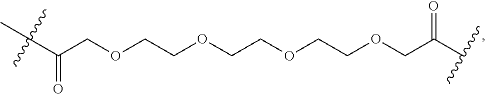 [SMILES: *C(=O)COCCOCCOCCOCC(*)=O]

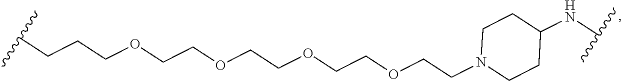 [SMILES: *CCCOCCOCCOCCOCCN1CCC(N*)CC1]

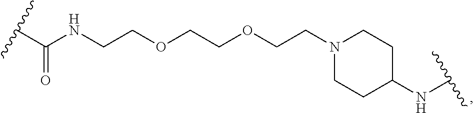 [SMILES: *NC1CCN(CCOCCOCCNC(*)=O)CC1]

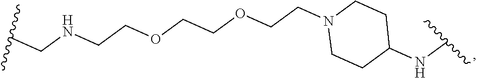 [SMILES: *CNCCOCCOCCN1CCC(N*)CC1]

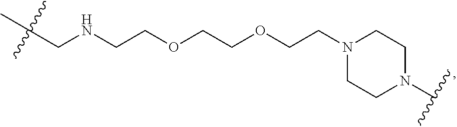 [SMILES: *CNCCOCCOCCN1CCN(*)CC1]

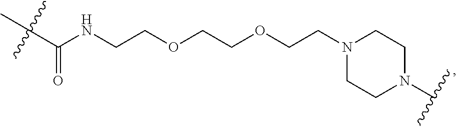 [SMILES: *C(=O)NCCOCCOCCN1CCN(*)CC1]

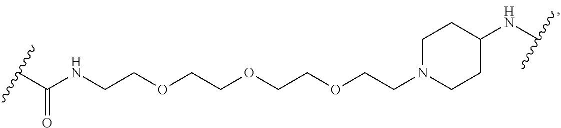 [SMILES: *NC1CCN(CCOCCOCCOCCNC(*)=O)CC1]

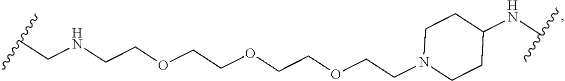 [SMILES: *CNCCOCCOCCOCCN1CCC(N*)CC1]

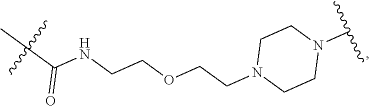 [SMILES: *C(=O)NCCOCCN1CCN(*)CC1]

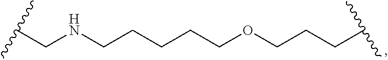 [SMILES: *CCCOCCCCCNC*]

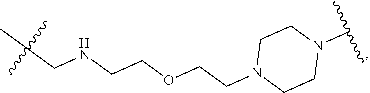 [SMILES: *CNCCOCCN1CCN(*)CC1]

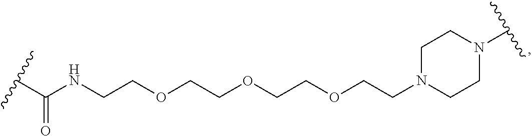 [SMILES: *C(=O)NCCOCCOCCOCCN1CCN(*)CC1]

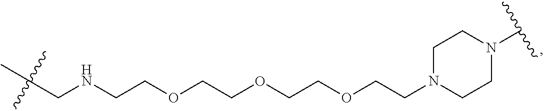 [SMILES: *CNCCOCCOCCOCCN1CCN(*)CC1]

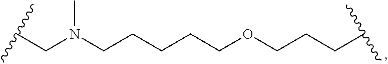 [SMILES: *CCCOCCCCCN(C)C*]

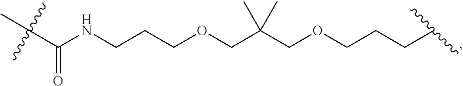 [SMILES: *CCCOCC(C)(C)COCCCNC(*)=O]

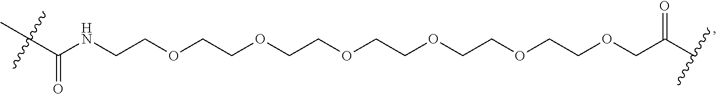 [SMILES: *C(=O)COCCOCCOCCOCCOCCOCCNC(*)=O]

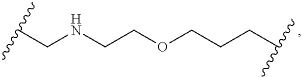 [SMILES: *CCCOCCNC*]

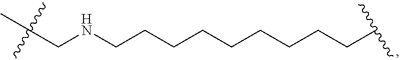 [SMILES: *CCCCCCCCCNC*]

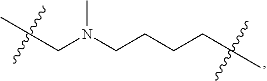 [SMILES: *CCCCN(C)C*]

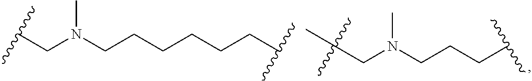

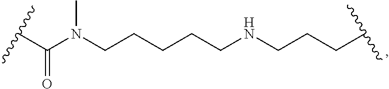 [SMILES: *CCCNCCCCCN(C)C(*)=O]

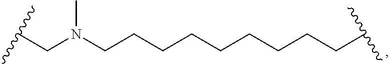 [SMILES: *CCCCCCCCCN(C)C*]

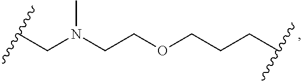 [SMILES: *CCCOCCN(C)C*]

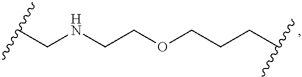 [SMILES: *CCCOCCNC*]

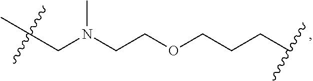 [SMILES: *CCCOCCN(C)C*]

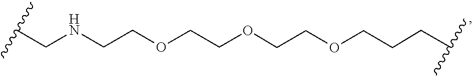 [SMILES: *CCCOCCOCCOCCNC*]

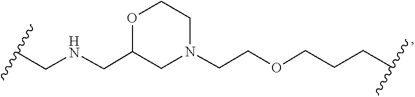 [SMILES: *CCCOCCN1CCOC(CNC*)C1]

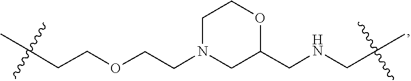 [SMILES: *CCOCCN1CCOC(CNC*)C1]

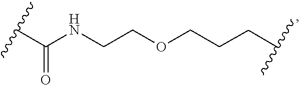 [SMILES: *CCCOCCNC(*)=O]

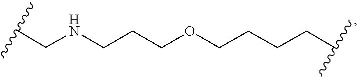 [SMILES: *CCCCOCCCNC*]

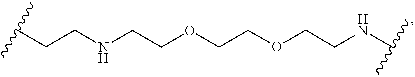 [SMILES: *CCNCCOCCOCCN*]

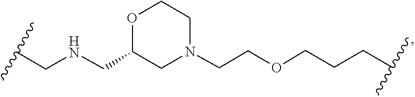 [SMILES: *CCCOCCN1CCO[C@@H](CNC*)C1]

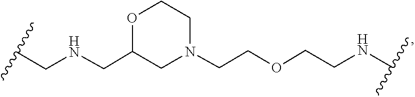 [SMILES: *CNCC1CN(CCOCCN*)CCO1]

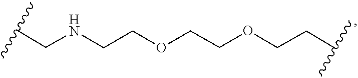 [SMILES: *CCOCCOCCNC*]

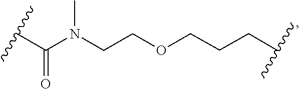 [SMILES: *CCCOCCN(C)C(*)=O]

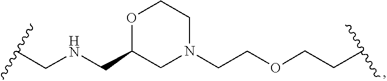 [SMILES: *CCOCCN1CCO[C@H](CNC*)C1]

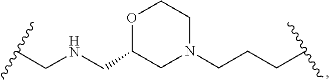 [SMILES: *CCCN1CCO[C@@H](CNC*)C1]

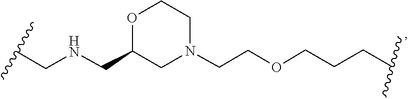 [SMILES: *CCCOCCN1CCO[C@H](CNC*)C1]

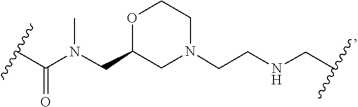 [SMILES: *CNCCN1CCO[C@H](CN(C)C(*)=O)C1]

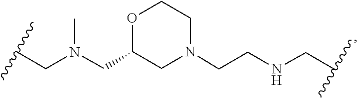 [SMILES: *CNCCN1CCO[C@@H](CN(C)C*)C1]

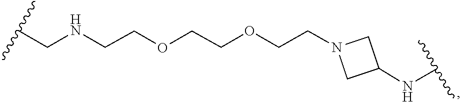 [SMILES: *CNCCOCCOCCN1CC(N*)C1]

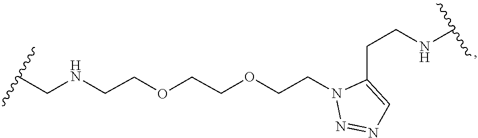 [SMILES: *CNCCOCCOCCn1nncc1CCN*]

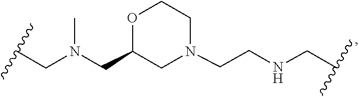 [SMILES: *CNCCN1CCO[C@H](CN(C)C*)C1]

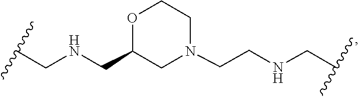 [SMILES: *CNCCN1CCO[C@H](CNC*)C1]

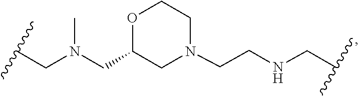 [SMILES: *CNCCN1CCO[C@@H](CN(C)C*)C1]

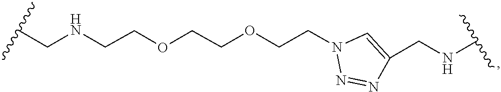 [SMILES: *CNCCOCCOCCn1cc(CN*)nn1]

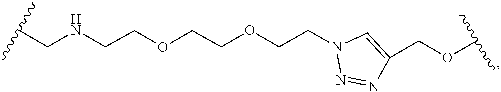 [SMILES: *CNCCOCCOCCn1cc(CO*)nn1]

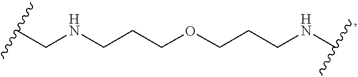 [SMILES: *CNCCCOCCCN*]

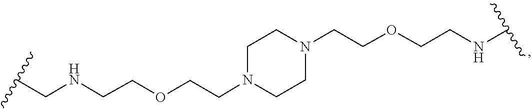 [SMILES: *CNCCOCCN1CCN(CCOCCN*)CC1]

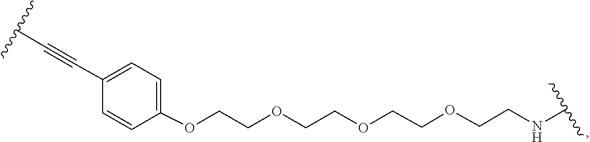 [SMILES: *C#Cc1ccc(OCCOCCOCCOCCN*)cc1]

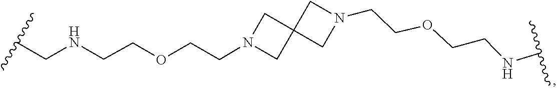 [SMILES: *CNCCOCCN1CC2(CN(CCOCCN*)C2)C1]

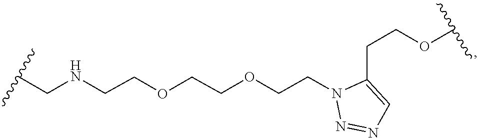 [SMILES: *CNCCOCCOCCn1nncc1CCO*]

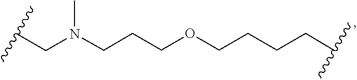 [SMILES: *CCCCOCCCN(C)C*]

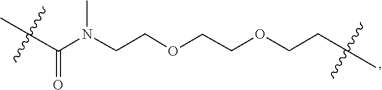 [SMILES: *CCOCCOCCN(C)C(*)=O]

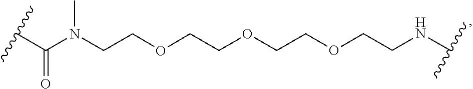 [SMILES: *NCCOCCOCCOCCN(C)C(*)=O]

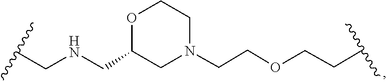 [SMILES: *CCOCCN1CCO[C@@H](CNC*)C1]

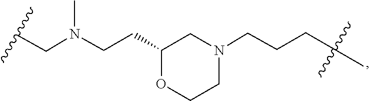 [SMILES: *CCCN1CCO[C@H](CCN(C)C*)C1]

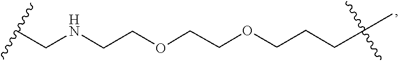 [SMILES: *CCCOCCOCCNC*]

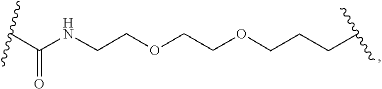 [SMILES: *CCCOCCOCCNC(*)=O]

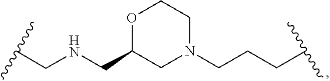 [SMILES: *CCCN1CCO[C@H](CNC*)C1]

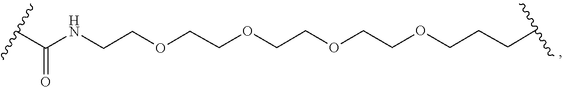 [SMILES: *CCCOCCOCCOCCOCCNC(*)=O]

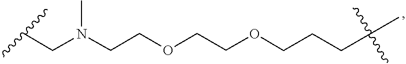 [SMILES: *CCCOCCOCCN(C)C*]

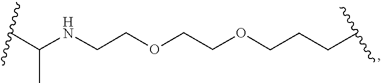 [SMILES: *CCCOCCOCCNC(*)C]

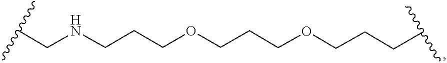 [SMILES: *CCCOCCCOCCCNC*]

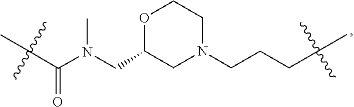 [SMILES: *CCCN1CCO[C@@H](CN(C)C(*)=O)C1]

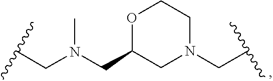 [SMILES: *CN(C)C[C@@H]1CN(C*)CCO1]

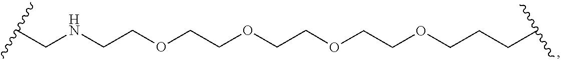 [SMILES: *CCCOCCOCCOCCOCCNC*]

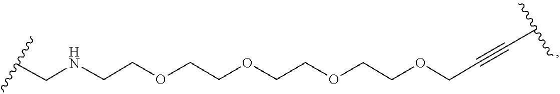 [SMILES: *C#CCOCCOCCOCCOCCNC*]

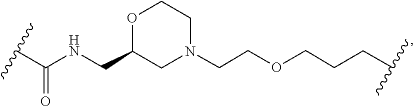 [SMILES: *CCCOCCN1CCO[C@H](CNC(*)=O)C1]

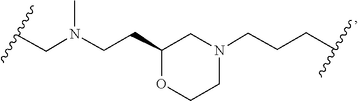 [SMILES: *CCCN1CCO[C@@H](CCN(C)C*)C1]

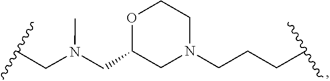 [SMILES: *CCCN1CCO[C@@H](CN(C)C*)C1]

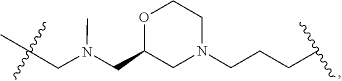 [SMILES: *CCCN1CCO[C@H](CN(C)C*)C1]

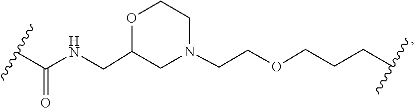 [SMILES: *CCCOCCN1CCOC(CNC(*)=O)C1]

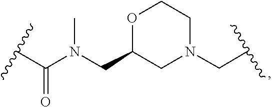 [SMILES: *CN1CCO[C@H](CN(C)C(*)=O)C1]

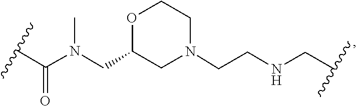 [SMILES: *CNCCN1CCO[C@@H](CN(C)C(*)=O)C1]

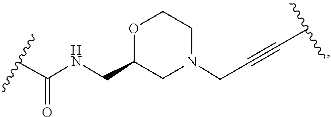 [SMILES: *C#CCN1CCO[C@H](CNC(*)=O)C1]

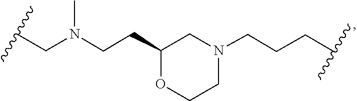 [SMILES: *CCCN1CCO[C@@H](CCN(C)C*)C1]

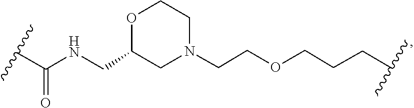 [SMILES: *CCCOCCN1CCO[C@@H](CNC(*)=O)C1]

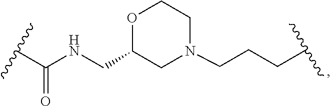 [SMILES: *CCCN1CCO[C@@H](CNC(*)=O)C1]

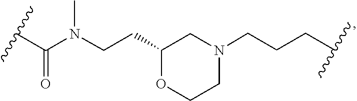 [SMILES: *CCCN1CCO[C@H](CCN(C)C(*)=O)C1]

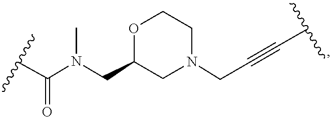 [SMILES: *C#CCN1CCO[C@H](CN(C)C(*)=O)C1]

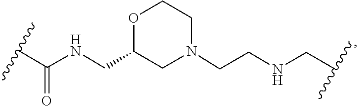 [SMILES: *CNCCN1CCO[C@@H](CNC(*)=O)C1]

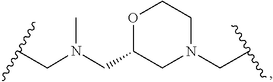 [SMILES: *CN(C)C[C@H]1CN(C*)CCO1]

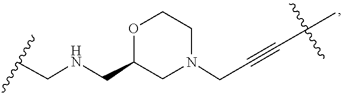 [SMILES: *C#CCN1CCO[C@H](CNC*)C1]

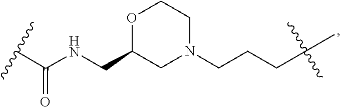 [SMILES: *CCCN1CCO[C@H](CNC(*)=O)C1]

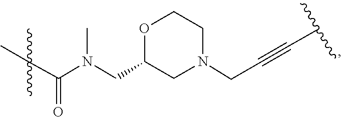 [SMILES: *C#CCN1CCO[C@@H](CN(C)C(*)=O)C1]

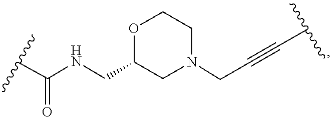 [SMILES: *C#CCN1CCO[C@@H](CNC(*)=O)C1]

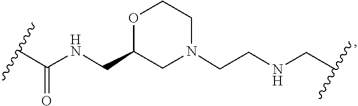 [SMILES: *CNCCN1CCO[C@H](CNC(*)=O)C1]

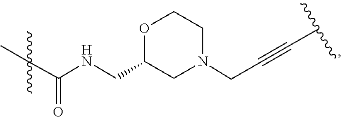 [SMILES: *C#CCN1CCO[C@@H](CNC(*)=O)C1]

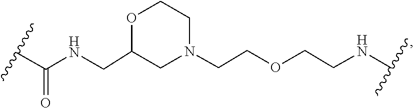 [SMILES: *NCCOCCN1CCOC(CNC(*)=O)C1]

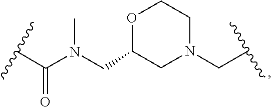 [SMILES: *CN1CCO[C@@H](CN(C)C(*)=O)C1]

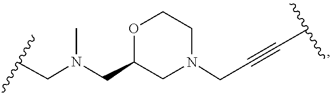 [SMILES: *C#CCN1CCO[C@H](CN(C)C*)C1]

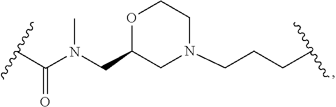 [SMILES: *CCCN1CCO[C@H](CN(C)C(*)=O)C1]

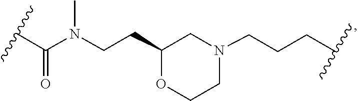 [SMILES: *CCCN1CCO[C@@H](CCN(C)C(*)=O)C1]

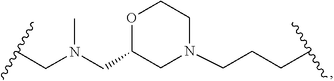 [SMILES: *CCCN1CCO[C@@H](CN(C)C*)C1]

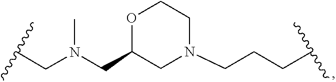 [SMILES: *CCCN1CCO[C@H](CN(C)C*)C1]

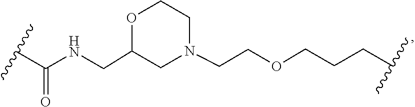 [SMILES: *CCCOCCN1CCOC(CNC(*)=O)C1]

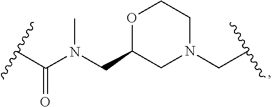 [SMILES: *CN1CCO[C@H](CN(C)C(*)=O)C1]

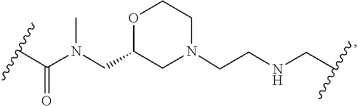 [SMILES: *CNCCN1CCO[C@@H](CN(C)C(*)=O)C1]

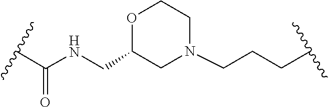 [SMILES: *CCCN1CCO[C@@H](CNC(*)=O)C1]

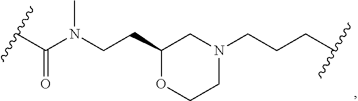 [SMILES: *CCCN1CCO[C@@H](CCN(C)C(*)=O)C1]

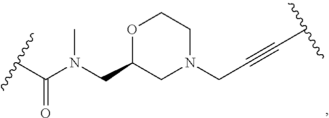 [SMILES: *C#CCN1CCO[C@H](CN(C)C(*)=O)C1]

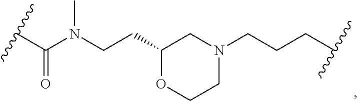 [SMILES: *CCCN1CCO[C@H](CCN(C)C(*)=O)C1]

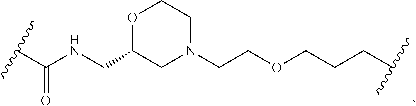 [SMILES: *CCCOCCN1CCO[C@@H](CNC(*)=O)C1]

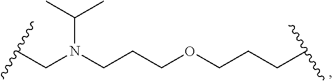 [SMILES: *CCCOCCCN(C*)C(C)C]

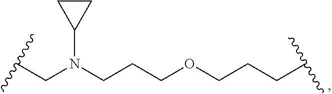 [SMILES: *CCCOCCCN(C*)C1CC1]

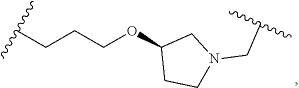 [SMILES: *CCCO[C@@H]1CCN(C*)C1]

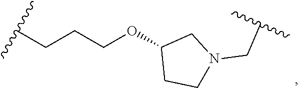 [SMILES: *CCCO[C@H]1CCN(C*)C1]

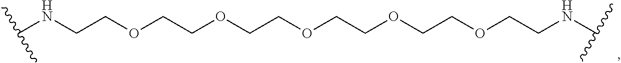 [SMILES: *NCCOCCOCCOCCOCCOCCN*]

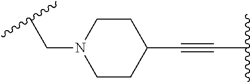 [SMILES: *C#CC1CCN(C*)CC1]

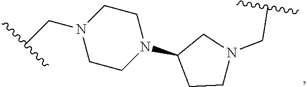 [SMILES: *CN1CCN([C@@H]2CCN(C*)C2)CC1]

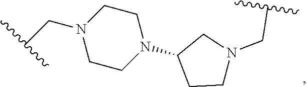 [SMILES: *CN1CCN([C@H]2CCN(C*)C2)CC1]

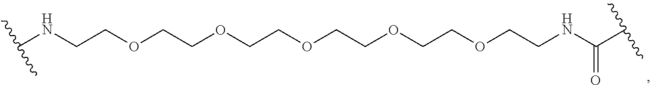 [SMILES: *NCCOCCOCCOCCOCCOCCNC(*)=O]

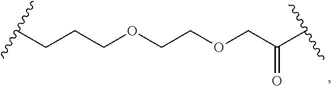 [SMILES: *CCCOCCOCC(*)=O]

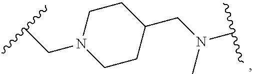 [SMILES: *CN1CCC(CN(*)C)CC1]

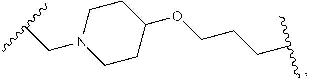 [SMILES: *CCCOC1CCN(C*)CC1]

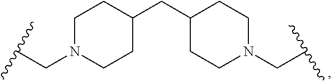 [SMILES: *CN1CCC(CC2CCN(C*)CC2)CC1]

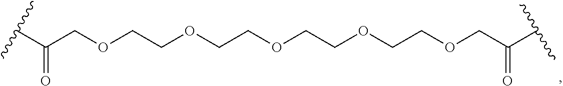 [SMILES: *C(=O)COCCOCCOCCOCCOCC(*)=O]

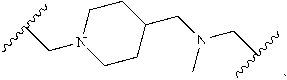 [SMILES: *CN(C)CC1CCN(C*)CC1]

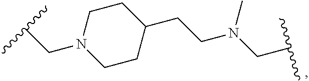 [SMILES: *CN(C)CCC1CCN(C*)CC1]

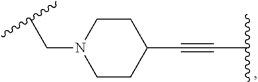 [SMILES: *C#CC1CCN(C*)CC1]

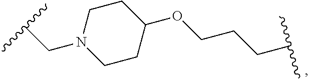 [SMILES: *CCCOC1CCN(C*)CC1]

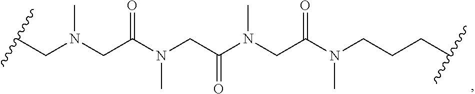 [SMILES: *CCCN(C)C(=O)CN(C)C(=O)CN(C)C(=O)CN(C)C*]

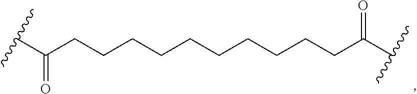 [SMILES: *C(=O)CCCCCCCCCCC(*)=O]

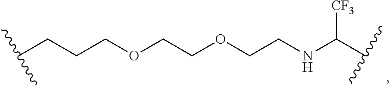 [SMILES: *CCCOCCOCCNC(*)C(F)(F)F]

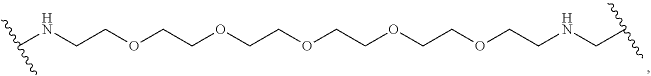 [SMILES: *CNCCOCCOCCOCCOCCOCCN*]

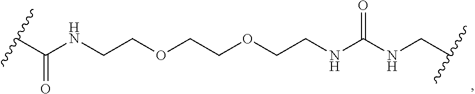 [SMILES: *CNC(=O)NCCOCCOCCNC(*)=O]

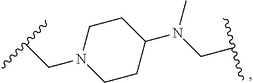 [SMILES: *CN1CCC(N(C)C*)CC1]

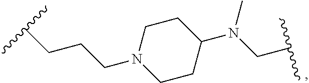 [SMILES: *CCCN1CCC(N(C)C*)CC1]

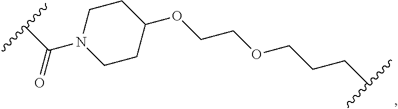 [SMILES: *CCCOCCOC1CCN(C(*)=O)CC1]

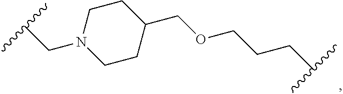 [SMILES: *CCCOCC1CCN(C*)CC1]

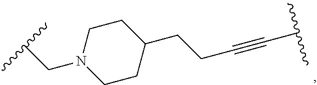 [SMILES: *C#CCCC1CCN(C*)CC1]

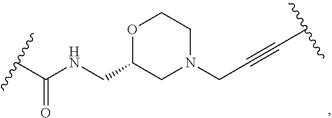 [SMILES: *C#CCN1CCO[C@@H](CNC(*)=O)C1]

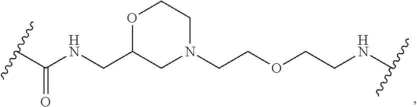 [SMILES: *NCCOCCN1CCOC(CNC(*)=O)C1]

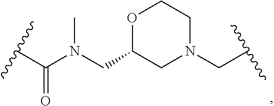 [SMILES: *CN1CCO[C@@H](CN(C)C(*)=O)C1]

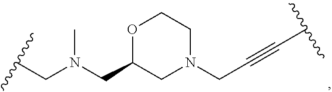 [SMILES: *C#CCN1CCO[C@H](CN(C)C*)C1]

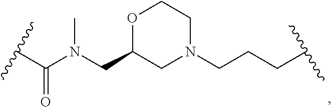 [SMILES: *CCCN1CCO[C@H](CN(C)C(*)=O)C1]

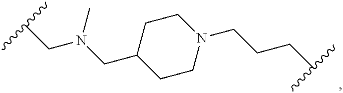 [SMILES: *CCCN1CCC(CN(C)C*)CC1]

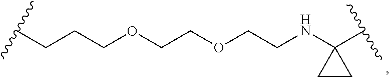 [SMILES: *CCCOCCOCCNC1(*)CC1]

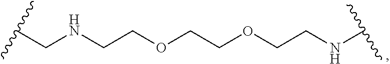 [SMILES: *CNCCOCCOCCN*]

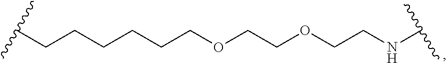 [SMILES: *CCCCCCOCCOCCN*]

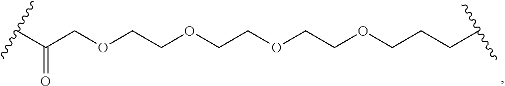 [SMILES: *CCCOCCOCCOCCOCC(*)=O]

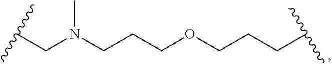 [SMILES: *CCCOCCCN(C)C*]

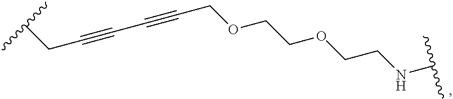 [SMILES: *CC#CC#CCOCCOCCN*]

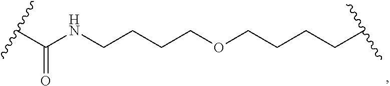 [SMILES: *CCCCOCCCCNC(*)=O]

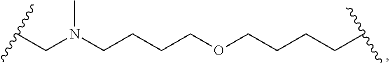 [SMILES: *CCCCOCCCCN(C)C*]

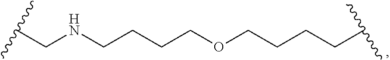 [SMILES: *CCCCOCCCCNC*]

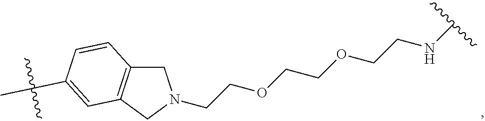 [SMILES: *NCCOCCOCCN1Cc2ccc(*)cc2C1]

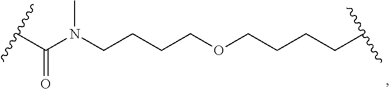 [SMILES: *CCCCOCCCCN(C)C(*)=O]

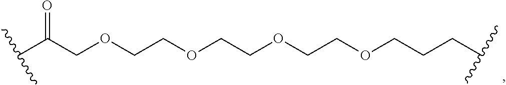 [SMILES: *CCCOCCOCCOCCOCC(*)=O]

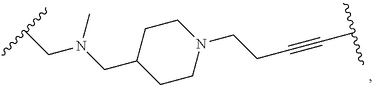 [SMILES: *C#CCCN1CCC(CN(C)C*)CC1]

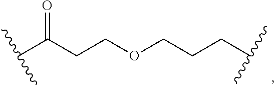 [SMILES: *CCCOCCC(*)=O]

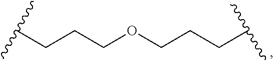 [SMILES: *CCCOCCC*]

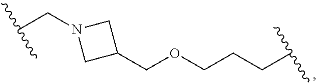 [SMILES: *CCCOCC1CN(C*)C1]

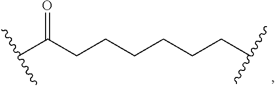 [SMILES: *CCCCCCC(*)=O]

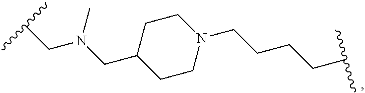 [SMILES: *CCCCN1CCC(CN(C)C*)CC1]

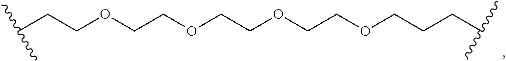 [SMILES: *CCCOCCOCCOCCOCC*]

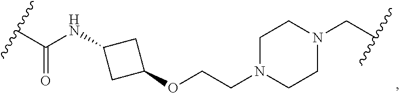 [SMILES: *CN1CCN(CCO[C@H]2C[C@H](NC(*)=O)C2)CC1]

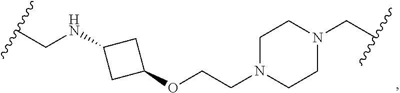 [SMILES: *CN[C@H]1C[C@H](OCCN2CCN(C*)CC2)C1]

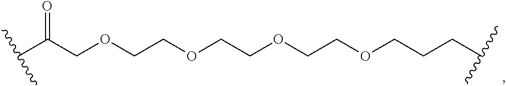 [SMILES: *CCCOCCOCCOCCOCC(*)=O]

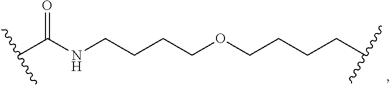 [SMILES: *CCCCOCCCCNC(*)=O]

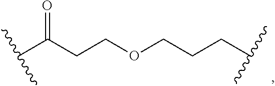 [SMILES: *CCCOCCC(*)=O]

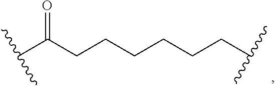 [SMILES: *CCCCCCC(*)=O]

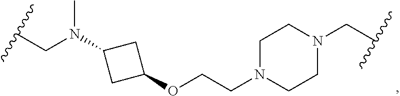 [SMILES: *CN1CCN(CCO[C@H]2C[C@H](N(C)C*)C2)CC1]

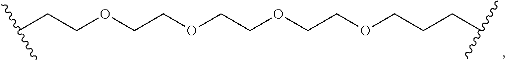 [SMILES: *CCCOCCOCCOCCOCC*]

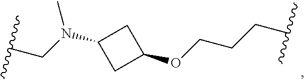 [SMILES: *CCCO[C@H]1C[C@H](N(C)C*)C1]

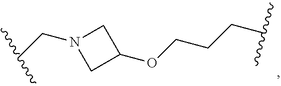 [SMILES: *CCCOC1CN(C*)C1]

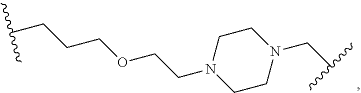 [SMILES: *CCCOCCN1CCN(C*)CC1]

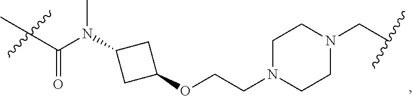 [SMILES: *CN1CCN(CCO[C@H]2C[C@H](N(C)C(*)=O)C2)CC1]

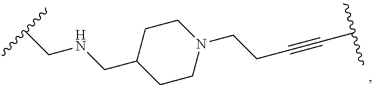 [SMILES: *C#CCCN1CCC(CNC*)CC1]

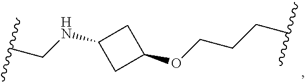 [SMILES: *CCCO[C@H]1C[C@H](NC*)C1]

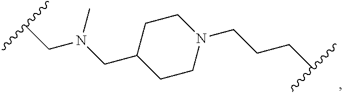 [SMILES: *CCCN1CCC(CN(C)C*)CC1]

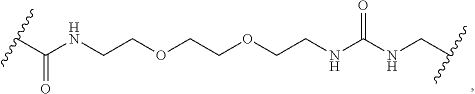 [SMILES: *CNC(=O)NCCOCCOCCNC(*)=O]

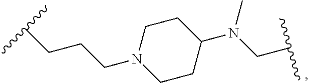 [SMILES: *CCCN1CCC(N(C)C*)CC1]

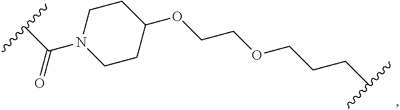 [SMILES: *CCCOCCOC1CCN(C(*)=O)CC1]

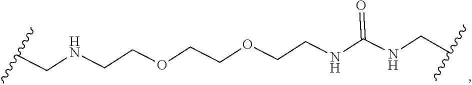 [SMILES: *CNCCOCCOCCNC(=O)NC*]

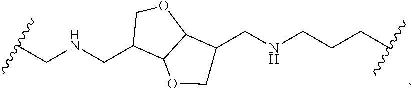 [SMILES: *CCCNCC1COC2C(CNC*)COC12]

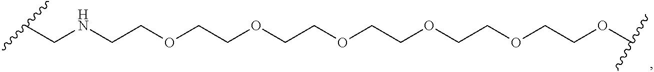 [SMILES: *CNCCOCCOCCOCCOCCOCCO*]

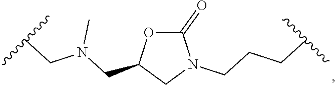 [SMILES: *CCCN1C[C@@H](CN(C)C*)OC1=O]

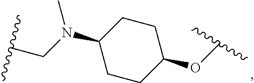 [SMILES: *CN(C)[C@H]1CC[C@@H](O*)CC1]

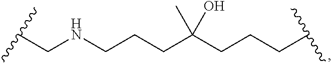 [SMILES: *CCCC(C)(O)CCCNC*]

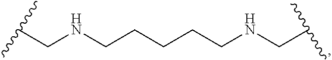 [SMILES: *CNCCCCCNC*]

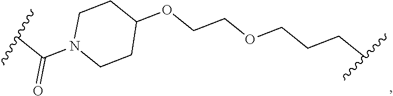 [SMILES: *CCCOCCOC1CCN(C(*)=O)CC1]

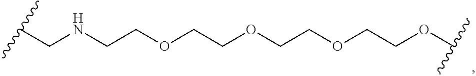 [SMILES: *CNCCOCCOCCOCCO*]

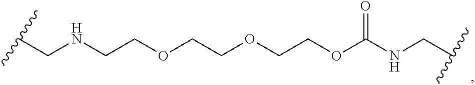 [SMILES: *CNCCOCCOCCOC(=O)NC*]

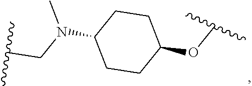 [SMILES: *CN(C)[C@H]1CC[C@H](O*)CC1]

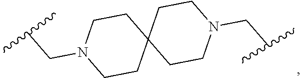 [SMILES: *CN1CCC2(CC1)CCN(C*)CC2]

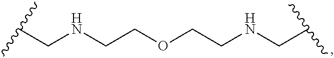 [SMILES: *CNCCOCCNC*]

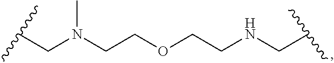 [SMILES: *CNCCOCCN(C)C*]

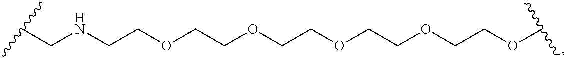 [SMILES: *CNCCOCCOCCOCCOCCO*]

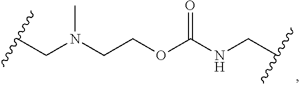 [SMILES: *CNC(=O)OCCN(C)C*]

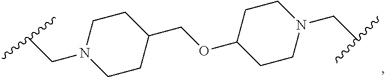 [SMILES: *CN1CCC(COC2CCN(C*)CC2)CC1]

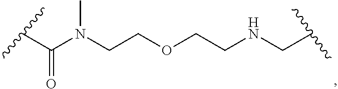 [SMILES: *CNCCOCCN(C)C(*)=O]

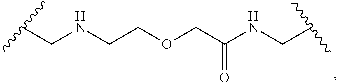 [SMILES: *CNCCOCC(=O)NC*]

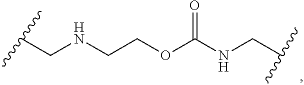 [SMILES: *CNCCOC(=O)NC*]

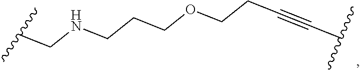 [SMILES: *C#CCCOCCCNC*]

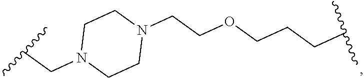 [SMILES: *CCCOCCN1CCN(C*)CC1]

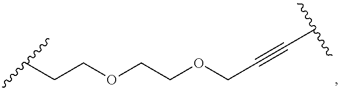 [SMILES: *C#CCOCCOCC*]

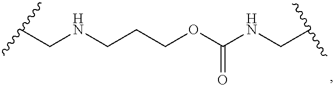 [SMILES: *CNCCCOC(=O)NC*]

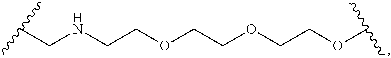 [SMILES: *CNCCOCCOCCO*]

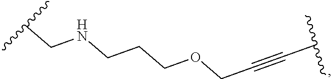 [SMILES: *C#CCOCCCNC*]

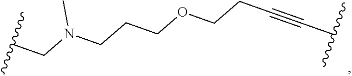 [SMILES: *C#CCCOCCCN(C)C*]

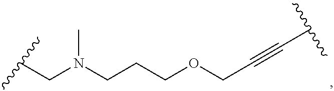 [SMILES: *C#CCOCCCN(C)C*]

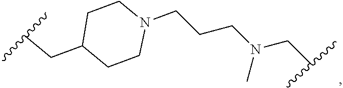 [SMILES: *CC1CCN(CCCN(C)C*)CC1]

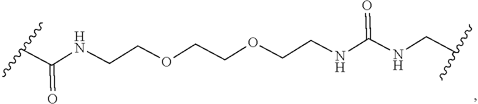 [SMILES: *CNC(=O)NCCOCCOCCNC(*)=O]

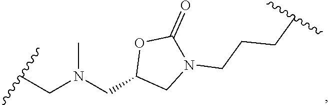 [SMILES: *CCCN1C[C@H](CN(C)C*)OC1=O]

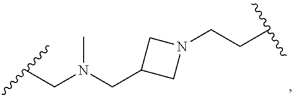 [SMILES: *CCN1CC(CN(C)C*)C1]

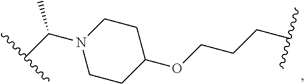 [SMILES: *CCCOC1CCN([C@H](*)C)CC1]

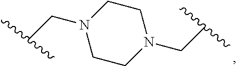 [SMILES: *CN1CCN(C*)CC1]

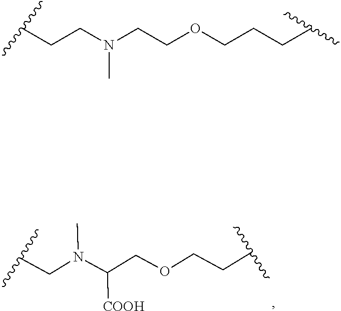 [SMILES: *CCCOCCN(C)CC* ; *CCOCC(C(=O)O)N(C)C*]

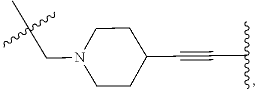 [SMILES: *C#CC1CCN(C*)CC1]

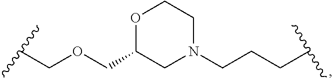 [SMILES: *CCCN1CCO[C@@H](COC*)C1]

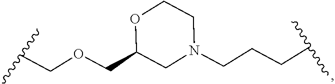 [SMILES: *CCCN1CCO[C@H](COC*)C1]

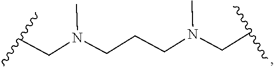 [SMILES: *CN(C)CCCN(C)C*]

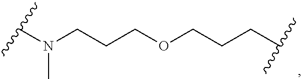 [SMILES: *CCCOCCCN(*)C]

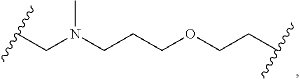 [SMILES: *CCOCCCN(C)C*]

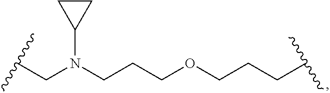 [SMILES: *CCCOCCCN(C*)C1CC1]

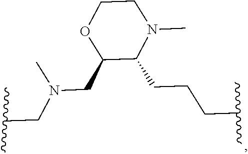 [SMILES: *CCC[C@@H]1[C@@H](CN(C)C*)OCCN1C]

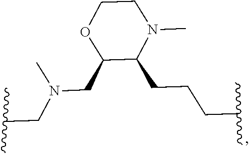 [SMILES: *CCC[C@H]1[C@@H](CN(C)C*)OCCN1C]

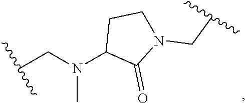 [SMILES: *CN1CCC(N(C)C*)C1=O]

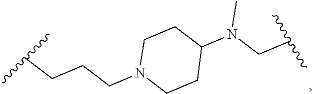 [SMILES: *CCCN1CCC(N(C)C*)CC1]

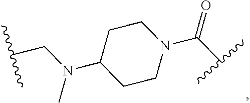 [SMILES: *CN(C)C1CCN(C(*)=O)CC1]

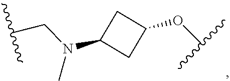 [SMILES: *CN(C)[C@H]1C[C@H](O*)C1]

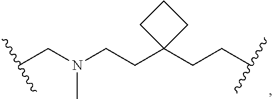 [SMILES: *CCC1(CCN(C)C*)CCC1]

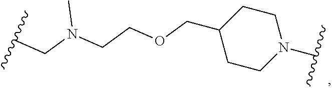 [SMILES: *CN(C)CCOCC1CCN(*)CC1]

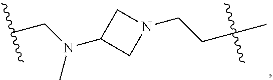 [SMILES: *CCN1CC(N(C)C*)C1]

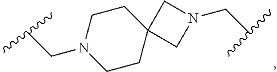 [SMILES: *CN1CCC2(CC1)CN(C*)C2]

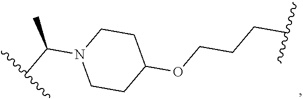 [SMILES: *CCCOC1CCN([C@@H](*)C)CC1]

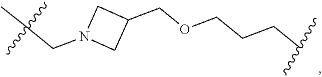 [SMILES: *CCCOCC1CN(C*)C1]

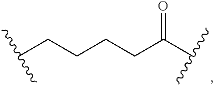 [SMILES: *CCCCC(*)=O]

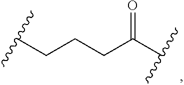 [SMILES: *CCCC(*)=O]

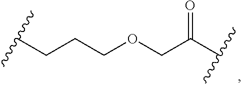 [SMILES: *CCCOCC(*)=O]

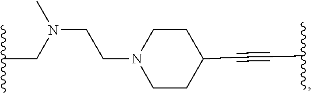 [SMILES: *C#CC1CCN(CCN(C)C*)CC1]

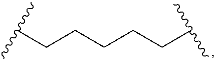 [SMILES: *CCCCC*]

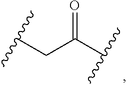 [SMILES: *CC(*)=O]

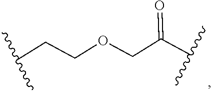 [SMILES: *CCOCC(*)=O]

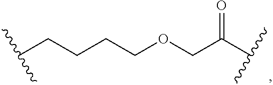 [SMILES: *CCCCOCC(*)=O]

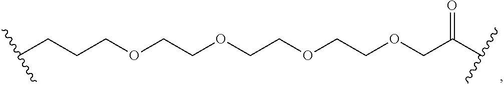 [SMILES: *CCCOCCOCCOCCOCC(*)=O]

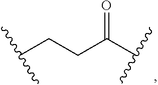 [SMILES: *CCC(*)=O]

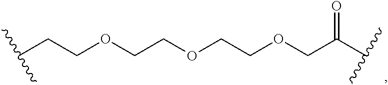 [SMILES: *CCOCCOCCOCC(*)=O]

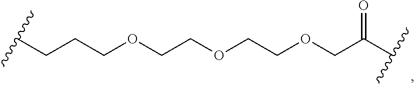 [SMILES: *CCCOCCOCCOCC(*)=O]

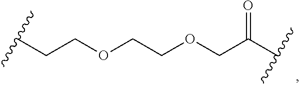 [SMILES: *CCOCCOCC(*)=O]

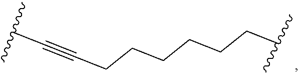 [SMILES: *C#CCCCCCC*]

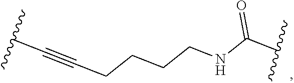 [SMILES: *C#CCCCCNC(*)=O]

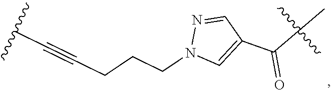 [SMILES: *C#CCCCn1cc(C(*)=O)cn1]

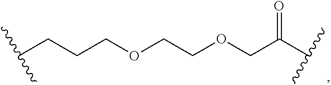 [SMILES: *CCCOCCOCC(*)=O]

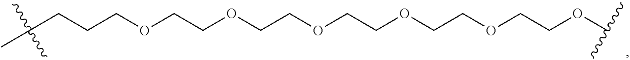 [SMILES: *CCCOCCOCCOCCOCCOCCO*]

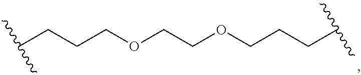 [SMILES: *CCCOCCOCCC*]

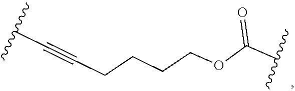 [SMILES: *C#CCCCCOC(*)=O]

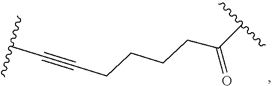 [SMILES: *C#CCCCCC(*)=O]

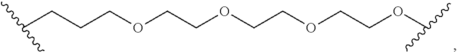 [SMILES: *CCCOCCOCCOCCO*]

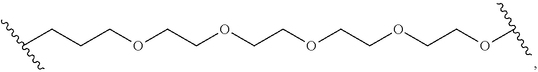 [SMILES: *CCCOCCOCCOCCOCCO*]

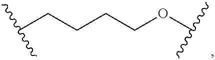 [SMILES: *CCCCO*]

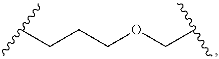 [SMILES: *CCCOC*]

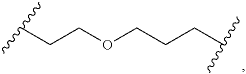 [SMILES: *CCCOCC*]

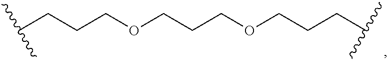 [SMILES: *CCCOCCCOCCC*]

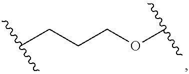 [SMILES: *CCCO*]

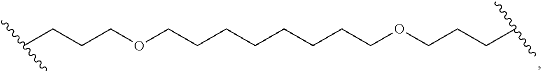 [SMILES: *CCCOCCCCCCCCOCCC*]

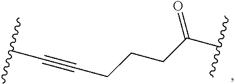 [SMILES: *C#CCCCC(*)=O]

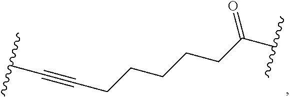 [SMILES: *C#CCCCCCC(*)=O]

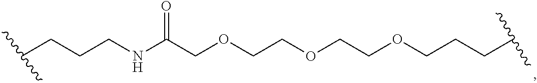 [SMILES: *CCCNC(=O)COCCOCCOCCC*]

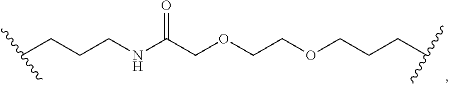 [SMILES: *CCCNC(=O)COCCOCCC*]

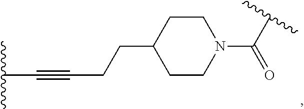 [SMILES: *C#CCCC1CCN(C(*)=O)CC1]

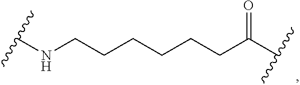 [SMILES: *NCCCCCCC(*)=O]

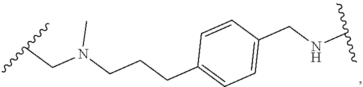 [SMILES: *CN(C)CCCc1ccc(CN*)cc1]

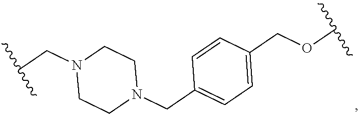 [SMILES: *CN1CCN(Cc2ccc(CO*)cc2)CC1]

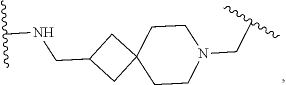 [SMILES: *CN1CCC2(CC1)CC(CN*)C2]

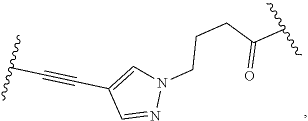 [SMILES: *C#Cc1cnn(CCCC(*)=O)c1]

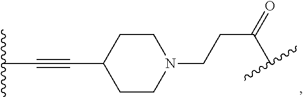 [SMILES: *C#CC1CCN(CCC(*)=O)CC1]

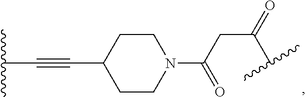 [SMILES: *C#CC1CCN(C(=O)CC(*)=O)CC1]

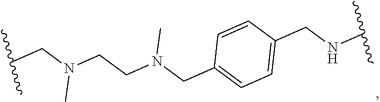 [SMILES: *CN(C)CCN(C)Cc1ccc(CN*)cc1]

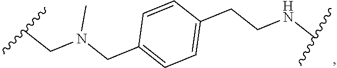 [SMILES: *CN(C)Cc1ccc(CCN*)cc1]

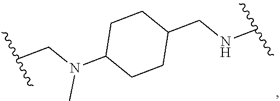 [SMILES: *CN(C)C1CCC(CN*)CC1]

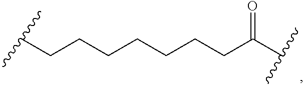 [SMILES: *CCCCCCCC(*)=O]

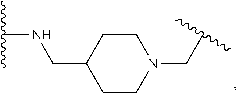 [SMILES: *CN1CCC(CN*)CC1]

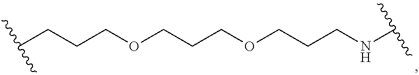 [SMILES: *CCCOCCCOCCCN*]

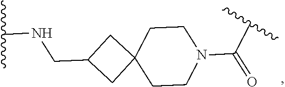 [SMILES: *NCC1CC2(CCN(C(*)=O)CC2)C1]

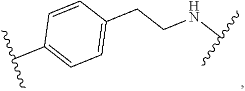 [SMILES: *NCCc1ccc(*)cc1]

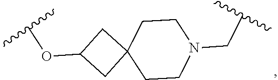 [SMILES: *CN1CCC2(CC1)CC(O*)C2]

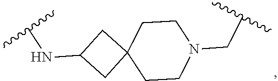 [SMILES: *CN1CCC2(CC1)CC(N*)C2]

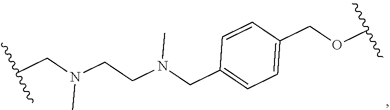 [SMILES: *CN(C)CCN(C)Cc1ccc(CO*)cc1]

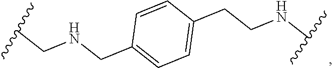 [SMILES: *CNCc1ccc(CCN*)cc1]

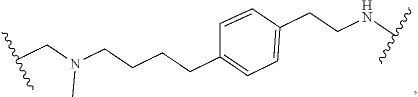 [SMILES: *CN(C)CCCCc1ccc(CCN*)cc1]

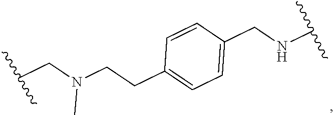 [SMILES: *CN(C)CCc1ccc(CN*)cc1]

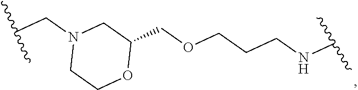 [SMILES: *CN1CCO[C@@H](COCCCN*)C1]

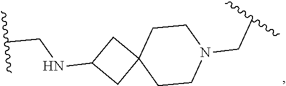 [SMILES: *CNC1CC2(CCN(C*)CC2)C1]

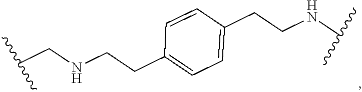 [SMILES: *CNCCc1ccc(CCN*)cc1]

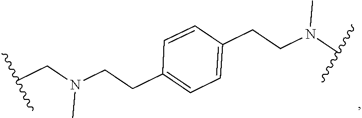 [SMILES: *CN(C)CCc1ccc(CCN(*)C)cc1]

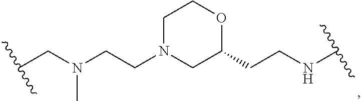 [SMILES: *CN(C)CCN1CCO[C@H](CCN*)C1]

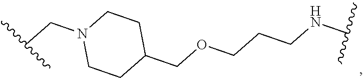 [SMILES: *CN1CCC(COCCCN*)CC1]

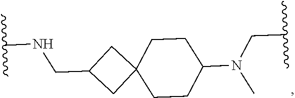 [SMILES: *CN(C)C1CCC2(CC1)CC(CN*)C2]

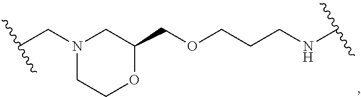 [SMILES: *CN1CCO[C@H](COCCCN*)C1]

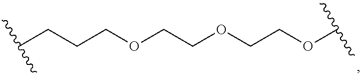 [SMILES: *CCCOCCOCCO*]

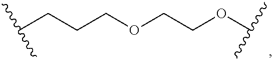 [SMILES: *CCCOCCO*]

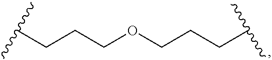 [SMILES: *CCCOCCC*]

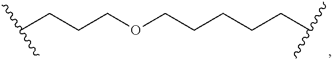 [SMILES: *CCCCCOCCC*]

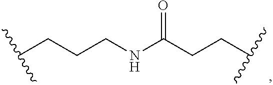 [SMILES: *CCCNC(=O)CC*]

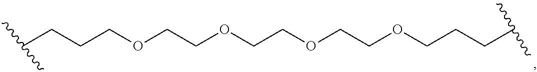 [SMILES: *CCCOCCOCCOCCOCCC*]

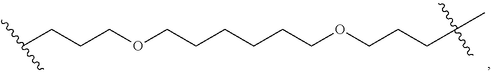 [SMILES: *CCCOCCCCCCOCCC*]

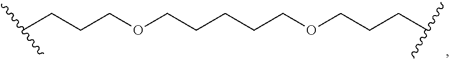 [SMILES: *CCCOCCCCCOCCC*]

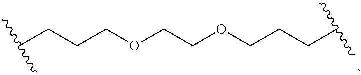 [SMILES: *CCCOCCOCCC*]

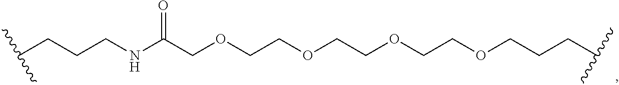 [SMILES: *CCCNC(=O)COCCOCCOCCOCCC*]

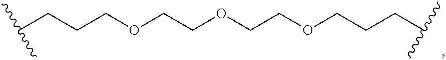 [SMILES: *CCCOCCOCCOCCC*]

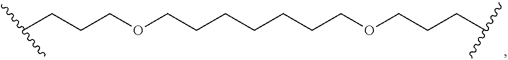 [SMILES: *CCCOCCCCCCCOCCC*]

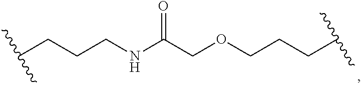 [SMILES: *CCCNC(=O)COCCC*]

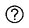

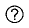

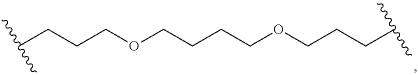 [SMILES: *CCCOCCCCOCCC*]

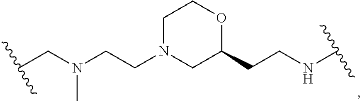 [SMILES: *CN(C)CCN1CCO[C@@H](CCN*)C1]

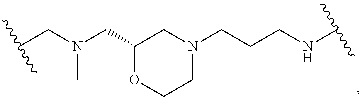 [SMILES: *CN(C)C[C@@H]1CN(CCCN*)CCO1]

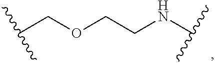 [SMILES: *COCCN*]

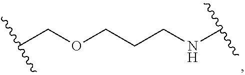 [SMILES: *COCCCN*]

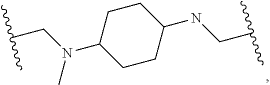 [SMILES: *CNC1CCC(N(C)C*)CC1]

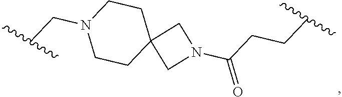 [SMILES: *CCC(=O)N1CC2(CCN(C*)CC2)C1]

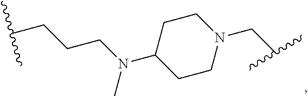 [SMILES: *CCCN(C)C1CCN(C*)CC1]

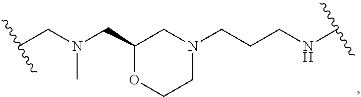 [SMILES: *CN(C)C[C@H]1CN(CCCN*)CCO1]

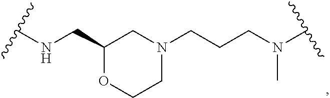 [SMILES: *NC[C@H]1CN(CCCN(*)C)CCO1]

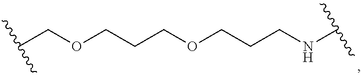 [SMILES: *COCCCOCCCN*]

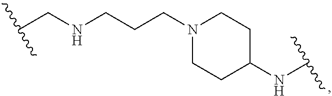 [SMILES: *CNCCCN1CCC(N*)CC1]

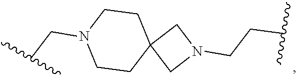 [SMILES: *CCN1CC2(CCN(C*)CC2)C1]

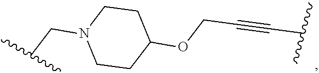 [SMILES: *C#CCOC1CCN(C*)CC1]

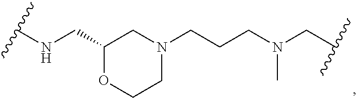 [SMILES: *CN(C)CCCN1CCO[C@H](CN*)C1]

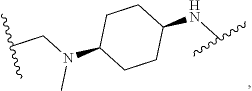 [SMILES: *CN(C)[C@H]1CC[C@@H](N*)CC1]

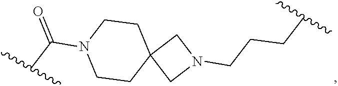 [SMILES: *CCCN1CC2(CCN(C(*)=O)CC2)C1]

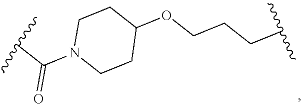 [SMILES: *CCCOC1CCN(C(*)=O)CC1]

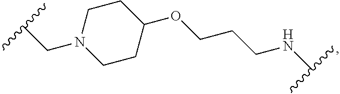 [SMILES: *CN1CCC(OCCCN*)CC1]

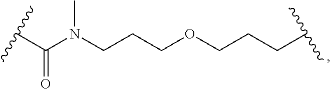 [SMILES: *CCCOCCCN(C)C(*)=O]

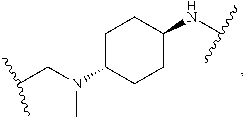 [SMILES: *CN(C)[C@H]1CC[C@H](N*)CC1]

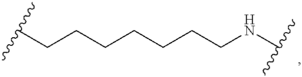 [SMILES: *CCCCCCCN*]

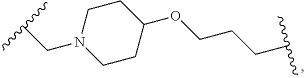 [SMILES: *CCCOC1CCN(C*)CC1]

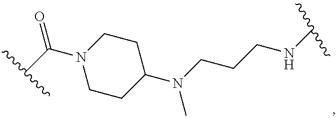 [SMILES: *NCCCN(C)C1CCN(C(*)=O)CC1]

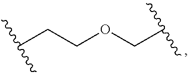 [SMILES: *CCOC*]

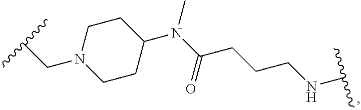 [SMILES: *CN1CCC(N(C)C(=O)CCCN*)CC1]

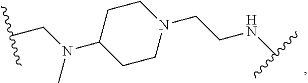 [SMILES: *CN(C)C1CCN(CCN*)CC1]

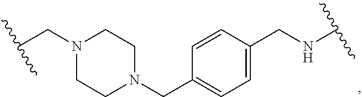 [SMILES: *CN1CCN(Cc2ccc(CN*)cc2)CC1]

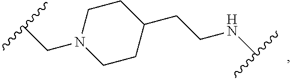 [SMILES: *CN1CCC(CCN*)CC1]

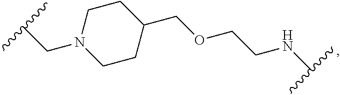 [SMILES: *CN1CCC(COCCN*)CC1]

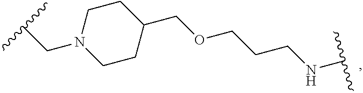 [SMILES: *CN1CCC(COCCCN*)CC1]

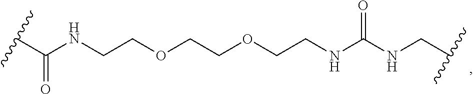 [SMILES: *CNC(=O)NCCOCCOCCNC(*)=O]

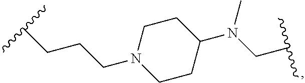 [SMILES: *CCCN1CCC(N(C)C*)CC1]

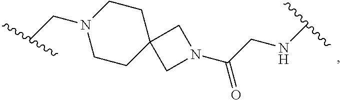 [SMILES: *CN1CCC2(CC1)CN(C(=O)CN*)C2]

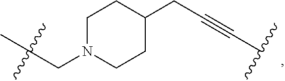 [SMILES: *C#CCC1CCN(C*)CC1]

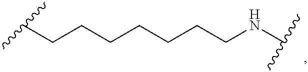 [SMILES: *CCCCCCCN*]

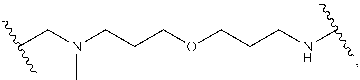 [SMILES: *CN(C)CCCOCCCN*]

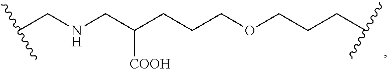 [SMILES: *CCCOCCCC(CNC*)C(=O)O]

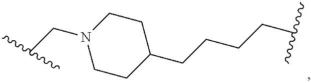 [SMILES: *CCCCC1CCN(C*)CC1]

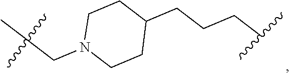 [SMILES: *CCCC1CCN(C*)CC1]

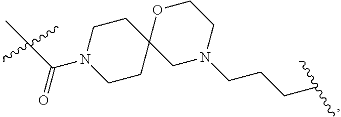 [SMILES: *CCCN1CCOC2(CCN(C(*)=O)CC2)C1]

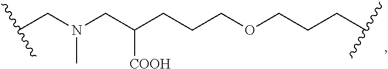 [SMILES: *CCCOCCCC(CN(C)C*)C(=O)O]

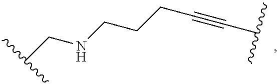 [SMILES: *C#CCCCNC*]

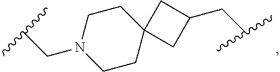 [SMILES: *CC1CC2(CCN(C*)CC2)C1]

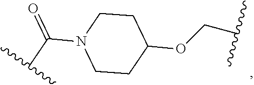 [SMILES: *COC1CCN(C(*)=O)CC1]

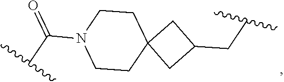 [SMILES: *CC1CC2(CCN(C(*)=O)CC2)C1]

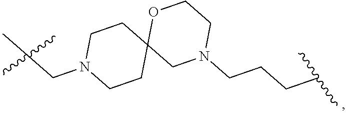 [SMILES: *CCCN1CCOC2(CCN(C*)CC2)C1]

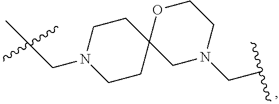 [SMILES: *CN1CCC2(CC1)CN(C*)CCO2]

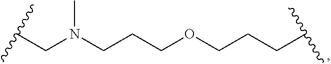 [SMILES: *CCCOCCCN(C)C*]

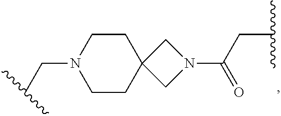 [SMILES: *CC(=O)N1CC2(CCN(C*)CC2)C1]

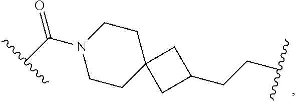 [SMILES: *CCC1CC2(CCN(C(*)=O)CC2)C1]

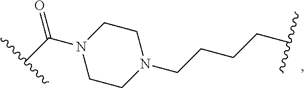 [SMILES: *CCCCN1CCN(C(*)=O)CC1]

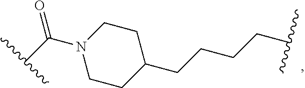 [SMILES: *CCCCC1CCN(C(*)=O)CC1]

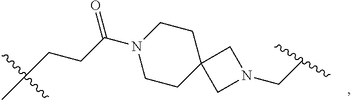 [SMILES: *CCC(=O)N1CCC2(CC1)CN(C*)C2]

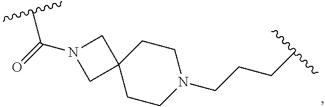 [SMILES: *CCCN1CCC2(CC1)CN(C(*)=O)C2]

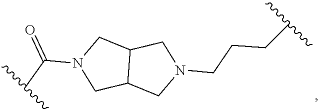 [SMILES: *CCCN1CC2CN(C(*)=O)CC2C1]

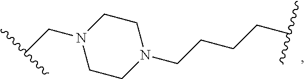 [SMILES: *CCCCN1CCN(C*)CC1]

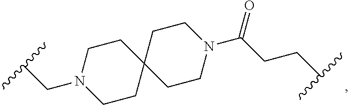 [SMILES: *CCC(=O)N1CCC2(CCN(C*)CC2)CC1]

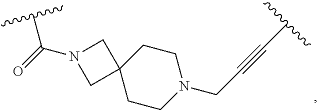 [SMILES: *C#CCN1CCC2(CC1)CN(C(*)=O)C2]

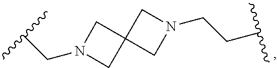 [SMILES: *CCN1CC2(CN(C*)C2)C1]

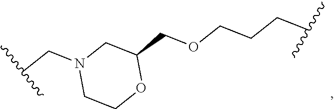 [SMILES: *CCCOC[C@@H]1CN(C*)CCO1]

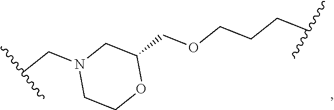 [SMILES: *CCCOC[C@H]1CN(C*)CCO1]

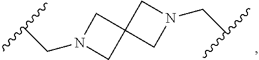 [SMILES: *CN1CC2(C1)CN(C*)C2]

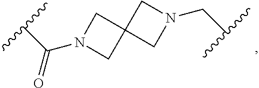 [SMILES: *CN1CC2(C1)CN(C(*)=O)C2]

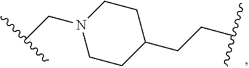 [SMILES: *CCC1CCN(C*)CC1]

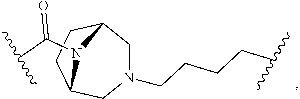 [SMILES: *CCCCN1C[C@H]2CC[C@@H](C1)N2C(*)=O]

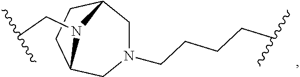 [SMILES: *CCCCN1C[C@H]2CC[C@@H](C1)N2C*]

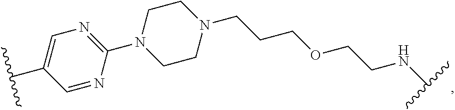 [SMILES: *NCCOCCCN1CCN(c2ncc(*)cn2)CC1]

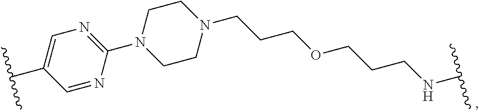 [SMILES: *NCCCOCCCN1CCN(c2ncc(*)cn2)CC1]

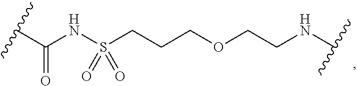 [SMILES: *NCCOCCCS(=O)(=O)NC(*)=O]

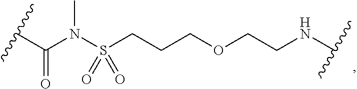 [SMILES: *NCCOCCCS(=O)(=O)N(C)C(*)=O]

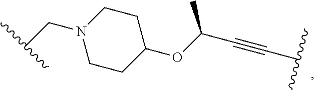 [SMILES: *C#C[C@H](C)OC1CCN(C*)CC1]

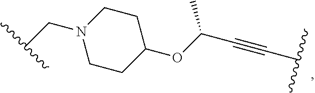 [SMILES: *C#C[C@@H](C)OC1CCN(C*)CC1]

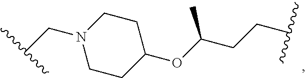 [SMILES: *CC[C@H](C)OC1CCN(C*)CC1]

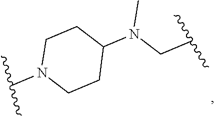 [SMILES: *CN(C)C1CCN(*)CC1]

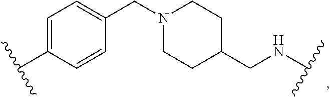 [SMILES: *NCC1CCN(Cc2ccc(*)cc2)CC1]

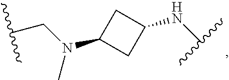 [SMILES: *CN(C)[C@H]1C[C@H](N*)C1]

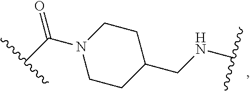 [SMILES: *NCC1CCN(C(*)=O)CC1]

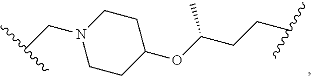 [SMILES: *CC[C@@H](C)OC1CCN(C*)CC1]

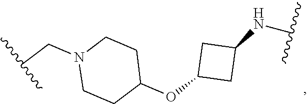 [SMILES: *CN1CCC(O[C@H]2C[C@H](N*)C2)CC1]

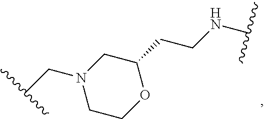 [SMILES: *CN1CCO[C@@H](CCN*)C1]

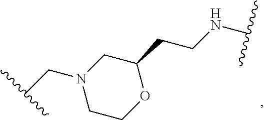 [SMILES: *CN1CCO[C@H](CCN*)C1]

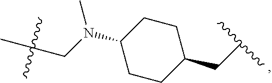 [SMILES: *CN(C)[C@H]1CC[C@H](C*)CC1]

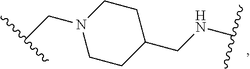 [SMILES: *CN1CCC(CN*)CC1]

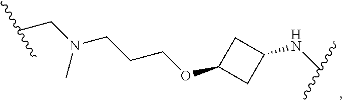 [SMILES: *CN(C)CCCO[C@H]1C[C@H](N*)C1]

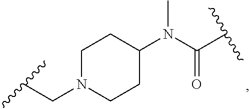 [SMILES: *CN1CCC(N(C)C(*)=O)CC1]

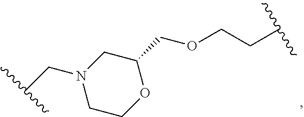 [SMILES: *CCOC[C@H]1CN(C*)CCO1]

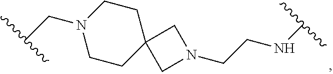 [SMILES: *CN1CCC2(CC1)CN(CCN*)C2]

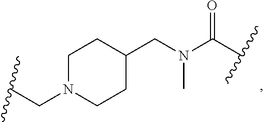 [SMILES: *CN1CCC(CN(C)C(*)=O)CC1]

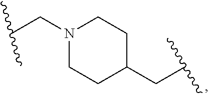 [SMILES: *CC1CCN(C*)CC1]

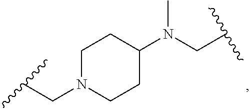 [SMILES: *CN1CCC(N(C)C*)CC1]

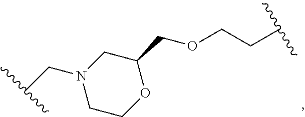 [SMILES: *CCOC[C@@H]1CN(C*)CCO1]

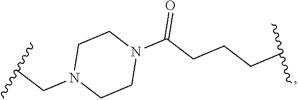 [SMILES: *CCCC(=O)N1CCN(C*)CC1]

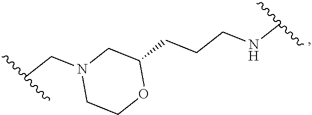 [SMILES: *CN1CCO[C@@H](CCCN*)C1]

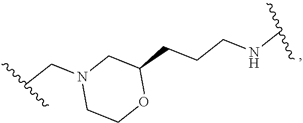 [SMILES: *CN1CCO[C@H](CCCN*)C1]

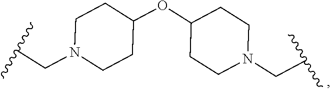 [SMILES: *CN1CCC(OC2CCN(C*)CC2)CC1]

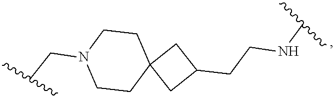 [SMILES: *CN1CCC2(CC1)CC(CCN*)C2]

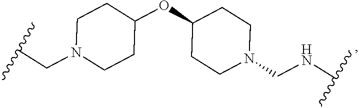

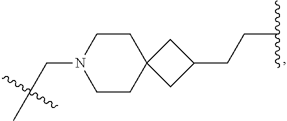 [SMILES: *CCC1CC2(CCN(C*)CC2)C1]

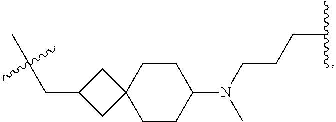 [SMILES: *CCCN(C)C1CCC2(CC1)CC(C*)C2]

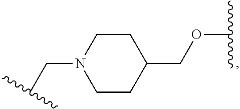 [SMILES: *CN1CCC(CO*)CC1]

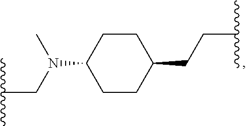 [SMILES: *CC[C@H]1CC[C@H](N(C)C*)CC1]

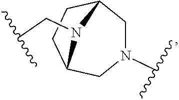 [SMILES: *CN1[C@@H]2CC[C@H]1CN(*)C2]

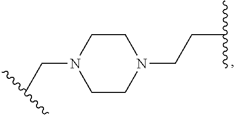 [SMILES: *CCN1CCN(C*)CC1]

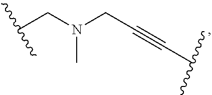 [SMILES: *C#CCN(C)C*]

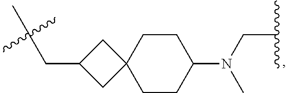 [SMILES: *CC1CC2(CCC(N(C)C*)CC2)C1]

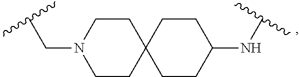 [SMILES: *CN1CCC2(CCC(N*)CC2)CC1]

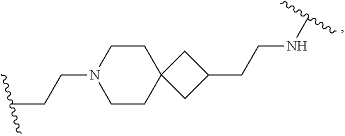 [SMILES: *CCN1CCC2(CC1)CC(CCN*)C2]

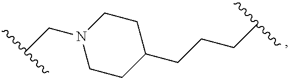 [SMILES: *CCCC1CCN(C*)CC1]

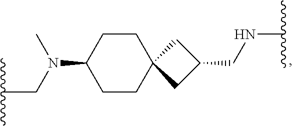

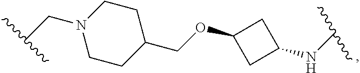 [SMILES: *CN1CCC(CO[C@H]2C[C@H](N*)C2)CC1]

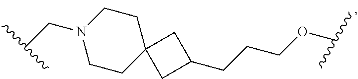 [SMILES: *CN1CCC2(CC1)CC(CCCO*)C2]

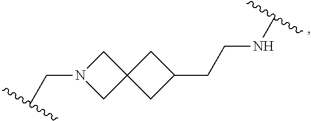 [SMILES: *CN1CC2(CC(CCN*)C2)C1]

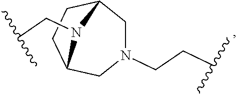 [SMILES: *CCN1C[C@H]2CC[C@@H](C1)N2C*]

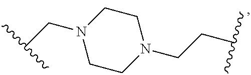 [SMILES: *CCN1CCN(C*)CC1]

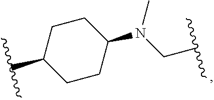 [SMILES: *CN(C)[C@H]1CC[C@@H](*)CC1]

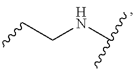

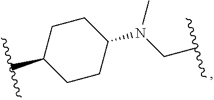 [SMILES: *CN(C)[C@H]1CC[C@H](*)CC1]

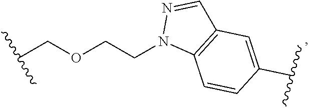 [SMILES: *COCCn1ncc2cc(*)ccc21]

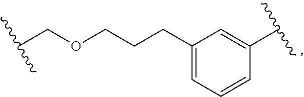 [SMILES: *COCCCc1cccc(*)c1]

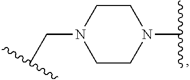 [SMILES: *CN1CCN(*)CC1]

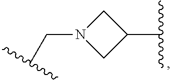 [SMILES: *CN1CC(*)C1]

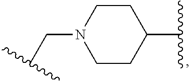 [SMILES: *CN1CCC(*)CC1]

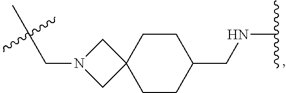 [SMILES: *CN1CC2(CCC(CN*)CC2)C1]

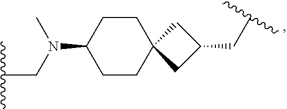

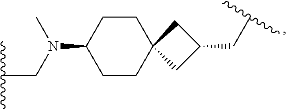

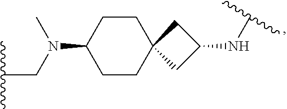

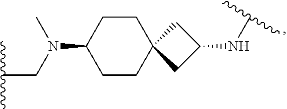

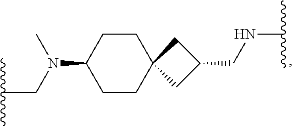

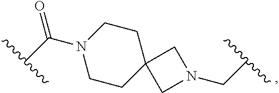 [SMILES: *CN1CC2(CCN(C(*)=O)CC2)C1]

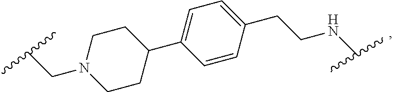 [SMILES: *CN1CCC(c2ccc(CCN*)cc2)CC1]

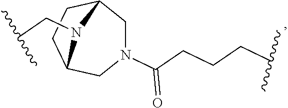 [SMILES: *CCCC(=O)N1C[C@H]2CC[C@@H](C1)N2C*]

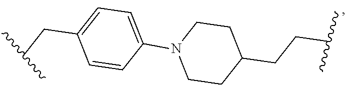 [SMILES: *CCC1CCN(c2ccc(C*)cc2)CC1]

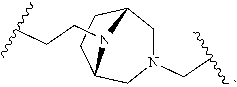 [SMILES: *CCN1[C@@H]2CC[C@H]1CN(C*)C2]

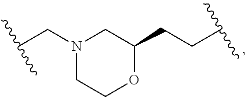 [SMILES: *CC[C@@H]1CN(C*)CCO1]

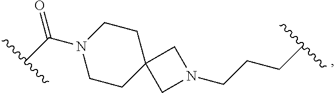 [SMILES: *CCCN1CC2(CCN(C(*)=O)CC2)C1]

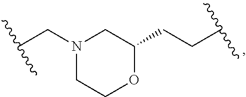 [SMILES: *CC[C@H]1CN(C*)CCO1]

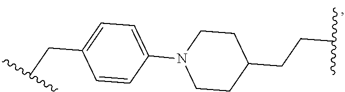 [SMILES: *CCC1CCN(c2ccc(C*)cc2)CC1]

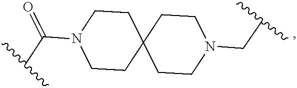 [SMILES: *CN1CCC2(CC1)CCN(C(*)=O)CC2]

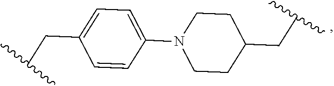 [SMILES: *Cc1ccc(N2CCC(C*)CC2)cc1]

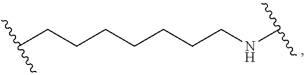 [SMILES: *CCCCCCCN*]

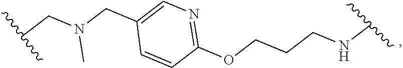 [SMILES: *CN(C)Cc1ccc(OCCCN*)nc1]

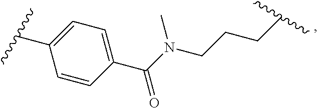 [SMILES: *CCCN(C)C(=O)c1ccc(*)cc1]

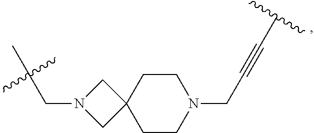 [SMILES: *C#CCN1CCC2(CC1)CN(C*)C2]

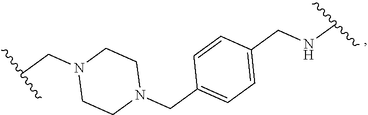 [SMILES: *CN1CCN(Cc2ccc(CN*)cc2)CC1]

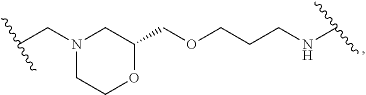 [SMILES: *CN1CCO[C@@H](COCCCN*)C1]

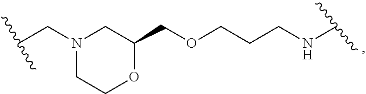 [SMILES: *CN1CCO[C@H](COCCCN*)C1]

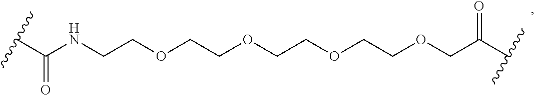 [SMILES: *C(=O)COCCOCCOCCOCCNC(*)=O]

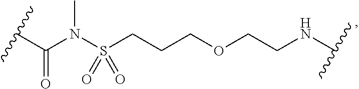 [SMILES: *NCCOCCCS(=O)(=O)N(C)C(*)=O]

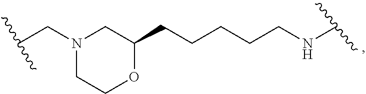 [SMILES: *CN1CCO[C@H](CCCCCN*)C1]

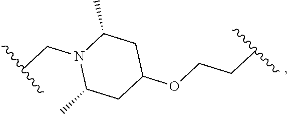 [SMILES: *CCOC1C[C@@H](C)N(C*)[C@@H](C)C1]

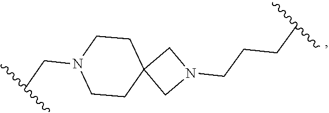 [SMILES: *CCCN1CC2(CCN(C*)CC2)C1]

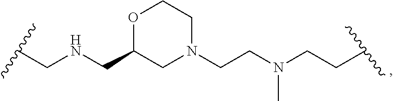 [SMILES: *CCN(C)CCN1CCO[C@H](CNC*)C1]

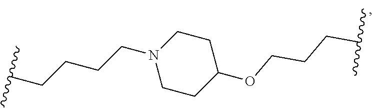 [SMILES: *CCCCN1CCC(OCCC*)CC1]

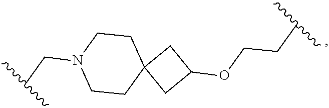 [SMILES: *CCOC1CC2(CCN(C*)CC2)C1]

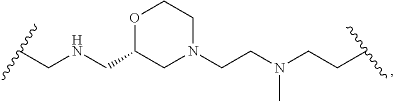 [SMILES: *CCN(C)CCN1CCO[C@@H](CNC*)C1]

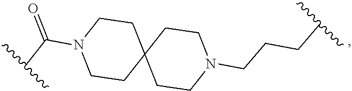 [SMILES: *CCCN1CCC2(CC1)CCN(C(*)=O)CC2]

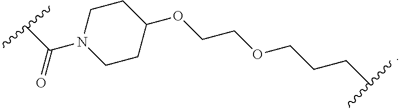 [SMILES: *CCCOCCOC1CCN(C(*)=O)CC1]

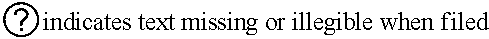

In a first embodiment of the twelfth and thirteenth aspects:
Z¹ is a bond, alkylene, —(CO)NR—, —(O-alkylene)ₐ-, -(alkylene-O)ₐ—, phenylene, or heterocyclylene, where each ring is optionally substituted with one or two alkyl;
Z² is a bond, alkylene, —(O-alkylene)_{b}-, -(alkylene-O)_{b}—, cycloalkylene, or heterocyclylene, where each ring is optionally substituted with one or two alkyl;
Z³ is a bond, alkylene, —C(O)NR—, —NR′(CO)—, —O—, —NR″—, cycloalkylene, phenylene, monocyclic heteroarylene, heterocyclylene, fused heterocyclylene, or spiro heterocyclylene, where each ring is optionally substituted with one or two alkyl;
Z⁴ is a bond, -(alkylene-NR″)—, —O—, —NR″—, cycloalkylene, phenylene, monocyclic heteroarylene, heterocyclylene, fused heterocyclylene, or spiro heterocyclylene, where each ring is optionally substituted with one or two alkyl;
Z⁵ is a bond; and
Z⁶ is —S(O)₂—; and
wherein each alkylene is optionally substituted with one, two, or three deuterium.

In a second embodiment of the twelfth and thirteenth aspects:
Z¹ is a bond;
Z² is a bond, alkylene, cycloalkylene, or heterocyclylene, where each ring is optionally substituted with one or two substituents independently selected from alkyl, alkoxy, halo, haloalkyl, and haloalkoxy;
Z³ is a bond, alkylene, —C(O)NR—, —NR′(CO)—, —O—, —NR″—, cycloalkylene, phenylene, monocyclic heteroarylene, heterocyclylene, bicyclic heterocyclylene, bridged heterocyclylene, fused heterocyclylene, or spiro heterocyclylene, where each ring is optionally substituted with one or two substituents independently selected from alkyl, alkoxy, halo, haloalkyl, and haloalkoxy;
Z⁴ is a bond, alkylene, —O—, cycloalkylene, phenylene, monocyclic heteroarylene, heterocyclylene, fused heterocyclylene, or spiro heterocyclylene, where each ring is optionally substituted with one or two substituents independently selected from alkyl, alkoxy, halo, haloalkyl, and haloalkoxy;
Z⁵ is phenylene, monocyclic heteroarylene, or heterocycylene, where each ring is optionally substituted with one or two substituents independently selected from alkyl, alkoxy, halo, haloalkyl, and haloalkoxy; and
Z⁶ is —S(O)₂—; and
wherein each alkylene is optionally substituted with one, two, or three deuterium.

In a third embodiment of the twelfth and thirteenth aspects and second embodiment thereof:
Z¹, and Z² are each a bond;
Z³ is cycloalkylene, phenylene, monocyclic heteroarylene, heterocyclylene, bicyclic heterocyclylene, bridged heterocyclylene, fused heterocyclylene, or spiro heterocyclylene, where each ring is optionally substituted with one or two substituents independently selected from alkyl, alkoxy, halo, haloalkyl, and haloalkoxy;
Z⁴ is a bond, alkylene, —O—, cycloalkylene, phenylene, monocyclic heteroarylene, heterocyclylene, fused heterocyclylene, or spiro heterocyclylene, where each ring is optionally substituted with one or two substituents independently selected from alkyl, alkoxy, halo, haloalkyl, and haloalkoxy;
Z⁵ is phenylene, monocyclic heteroarylene, or heterocycylene, where each ring is optionally substituted with one or two substituents independently selected from alkyl, alkoxy, halo, haloalkyl, and haloalkoxy; and
Z⁶ is —S(O)₂—; and
wherein alkylene is optionally substituted with one or two deuterium.

In a fourth embodiment of the twelfth and thirteenth aspects and second and third embodiments thereof:
Z¹, and Z² are each a bond;
Z³ is heterocyclylene, bicyclic heterocyclylene, bridged heterocyclylene, fused heterocyclylene, or spiro heterocyclylene, where each ring is optionally substituted with one or two substituents independently selected from alkyl, alkoxy, halo, haloalkyl, and haloalkoxy;
Z⁴ is alkylene, —O—, monocyclic heteroarylene, heterocyclylene, fused heterocyclylene, or spiro heterocyclylene, where each ring is optionally substituted with one or two substituents independently selected from alkyl, alkoxy, halo, haloalkyl, and haloalkoxy;
Z⁵ is phenylene, monocyclic heteroarylene, or heterocycylene, where each ring is optionally substituted with one or two substituents independently selected from alkyl, alkoxy, halo, haloalkyl, and haloalkoxy; and
Z⁶ is —S(O)₂—; and
wherein alkylene is optionally substituted with one or two deuterium.

In a fifth embodiment of the twelfth and thirteenth aspects and second to fourth embodiments thereof:
Z¹, and Z² are each a bond;
Z³ is heterocyclylene, bridged heterocyclylene, or spiro heterocyclylene, where each ring is optionally substituted with one or two substituents independently selected from alkyl, alkoxy, halo, haloalkyl, and haloalkoxy;
Z⁴ is alkylene, —O—, or heterocyclylene, where each ring is optionally substituted with one or two substituents independently selected from alkyl, alkoxy, halo, haloalkyl, and haloalkoxy;
Z⁵ is phenylene, monocyclic heteroarylene (e.g., pyridindiyl), or heterocycylene, where each ring is optionally substituted with one or two substituents independently selected from alkyl, alkoxy, halo, haloalkyl, and haloalkoxy; and
Z⁶ is —S(O)₂—; and
wherein alkylene is optionally substituted with one or two deuterium.

In a sixth embodiment of the twelfth and thirteenth aspects and second to fifth embodiments thereof:
Z¹, and Z² are each a bond;
Z³ is heterocyclylene, bridged heterocyclylene, or spiro heterocyclylene, where each ring is optionally substituted with one or two substituents independently selected from alkyl, alkoxy, halo, haloalkyl, and haloalkoxy;
Z⁴ is alkylene, —O—, or heterocyclylene, where each ring is optionally substituted with one or two substituents independently selected from alkyl, alkoxy, halo, haloalkyl, and haloalkoxy;
Z⁵ is phenylene optionally substituted with one or two substituents independently selected from alkyl, alkoxy, halo, haloalkyl, and haloalkoxy; and
Z⁶ is —S(O)₂—; and
wherein alkylene is optionally substituted with one or two deuterium

In a seventh embodiment of the twelfth and thirteenth aspects:
Z¹ is a bond;
Z² is cycloalkylene or heterocyclylene, where each ring is optionally substituted with one or two substituents independently selected from alkyl, alkoxy, halo, haloalkyl, and haloalkoxy;
Z³ is cycloalkylene, phenylene, monocyclic heteroarylene, heterocyclylene, bicyclic heterocyclylene, bridged heterocyclylene, fused heterocyclylene, or spiro heterocyclylene, where each ring is optionally substituted with one or two substituents independently selected from alkyl, alkoxy, halo, haloalkyl, and haloalkoxy;
Z⁴ is a bond, alkylene, or —O—;
Z⁵ is phenylene, monocyclic heteroarylene (e.g., pyridindiyl), or heterocycylene, where each ring is optionally substituted with one or two substituents independently selected from alkyl, alkoxy, halo, haloalkyl, and haloalkoxy; and
Z⁶ is —S(O)₂—.

In an eighth embodiment of the twelfth and thirteenth aspects and seventh embodiment thereof:
Z¹ is a bond;
Z² is heterocyclylene optionally substituted with one or two substituents independently selected from alkyl, alkoxy, halo, haloalkyl, and haloalkoxy;
Z³ is heterocyclylene optionally substituted with one or two substituents independently selected from alkyl, alkoxy, halo, haloalkyl, and haloalkoxy;
Z⁴ is a bond, alkylene, or —O—;
Z⁵ is phenylene optionally substituted with one or two substituents independently selected from alkyl, alkoxy, halo, haloalkyl, and haloalkoxy; and
Z⁶ is —S(O)₂—; and
wherein alkylene is optionally substituted with one or two deuterium

In a ninth embodiment of the twelfth and thirteenth aspects and seventh and eighth embodiments thereof:
Z¹ is a bond;
Z² is heterocyclylene, where each ring is optionally substituted with one or two substituents independently selected from alkyl, alkoxy, halo, haloalkyl, and haloalkoxy;
Z³ is a bond, alkylene, or —O—;
Z⁴ is heterocyclylene, bridged heterocyclylene, or spiro heterocyclylene, where each ring is optionally substituted with one or two substituents independently selected from alkyl, alkoxy, halo, haloalkyl, and haloalkoxy;
Z⁵ is phenylene optionally substituted with one or two substituents independently selected from alkyl, alkoxy, halo, haloalkyl, and haloalkoxy; and
Z⁶ is —S(O)₂—.

In a tenth embodiment of the twelfth and thirteenth aspects and first to ninth embodiments thereof:

### —Z⁵— is

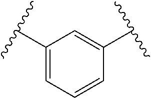 [SMILES: *c1cccc(*)c1]

(i.e., Z⁵ is phenylene where Z⁴ and Z⁶ are attached at meta position of the phenylene ring) optionally substituted with one or two substituents independently selected from alkyl, alkoxy, halo, haloalkyl, and haloalkoxy.

In an eleventh embodiment of the twelfth and thirteenth aspects and first to ninth embodiments thereof:
—Z⁵— is

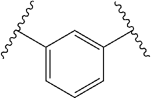 [SMILES: *c1cccc(*)c1]

optionally substituted with one or two substituents independently selected from methyl, methoxy, fluoro, chloro, difluoromethyl, trifluoromethyl, difluoromethoxy, and trifluoromethoxy.

In a twelfth embodiment of the twelfth and thirteenth aspects and first to ninth embodiments thereof:
—Z⁵— is

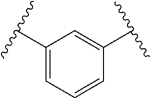 [SMILES: *c1cccc(*)c1]

optionally substituted with one or two substituents independently selected from methyl, fluoro, trifluoromethyl, and trifluoromethoxy.

In a thirteenth embodiment of the twelfth and thirteenth aspects and first to seventh embodiments thereof:
Z⁵ is pyridin-2,4-diyl, pyridin-2,6-diyl, or pyridin-3,5-diyl optionally substituted with one substituent selected from alkyl, alkoxy, halo, haloalkyl, and haloalkoxy.

In a fourteenth embodiment of the twelfth and thirteenth aspects and first to seventh embodiments thereof:
Z⁵ is pyridin-2,4-diyl, pyridin-2,6-diyl, or pyridin-3,5-diyl optionally substituted with one substituent selected from methyl, methoxy, fluoro, chloro, difluoromethyl, trifluoromethyl, difluoromethoxy, and trifluoromethoxy.

In a fifteenth embodiment of the twelfth and thirteenth aspects and first to fourteenth embodiments thereof: each alkylene of —Z¹-Z²—Z³—Z⁴-Z⁵—Z⁶—, by itself and when present, is methylene, ethylene, or propylene, each optionally substituted with one or two deuterium.

In a sixteenth embodiment of the twelfth and thirteenth aspects and first to fourteenth embodiments thereof: each alkylene of —Z¹-Z²—Z³—Z⁴-Z⁵—Z⁶—, by itself and when present, is methylene optionally substituted with one or two deuterium.

In a seventeenth embodiment of the twelfth and thirteenth aspects and first to sixteenth embodiments thereof: each alkylene of —Z¹-Z²—Z³—Z⁴-Z⁵—Z⁶—, by as part of another group and when present, (e.g., —(O-alkylene)ₐ, -(alkylene-O)ₐ—, -(alkylene-NR″)—) and when present, is ethylene or propylene.

In a eighteenth embodiment of the twelfth and thirteenth aspects and first to seventeenth embodiments thereof: each alkylene of —Z¹-Z²—Z³—Z⁴-Z⁵—Z⁶—, as part of another group (e.g., —(O-alkylene)ₐ, -(alkylene-O)ₐ—, -(alkylene-NR″)—) and when present, is ethylene.

In a nineteenth embodiment of the twelfth aspect and first to first to eighteenth, each R, R′ and R″ of —Z¹-Z²—Z³—Z⁴-Z⁵—Z⁶—, when present, is independently hydrogen or methyl.

In a twentieth embodiment of the twelfth and thirteenth aspects and first to nineteenth, each R, R′ and R″ of —Z¹-Z²—Z³—Z⁴-Z⁵—Z⁶—, when present, is hydrogen.

In a twenty-first embodiment of the twelfth and thirteenth aspects and first to first to nineteenth, each R, R′ and R″ of —Z¹-Z²—Z³—Z⁴-Z⁵—Z⁶—, when present, is methyl.

In a twenty-second embodiment of the twelfth and thirteenth aspects and first to twenty-first, each cycloalkylene of —Z¹-Z²—Z³—Z⁴-Z⁵—Z⁶—, when present, is independently selected from cyclopropylene, cyclobutylene, cyclopentylene, and cyclohexylene.

In a twenty-third embodiment of the twelfth and thirteenth aspects and first to twenty-second, each cycloalkylene of —Z¹-Z²—Z³—Z⁴-Z⁵—Z⁶—, when present, is independently selected from 1,3-cyclopentylene, 1,3-cyclohexylene, and 1,4-cyclohexylene.

In a twenty-fourth embodiment of the twelfth and thirteenth aspects and first to twenty-third, heteroarylene is monocyclic heteroarylene and each monocyclic heteroarylene of —Z¹-Z²—Z³—Z⁴-Z⁵—Z⁶—, when present, is independently selected from pyridindiyl and pyrimidinediyl unless stated otherwise in any of the embodiment above.

In a twenty-fifth embodiment of the twelfth and thirteenth aspects and first to twenty-fourth, heteroarylene is monocyclic heteroarylene and each monocyclic heteroarylene of —Z¹-Z²—Z³—Z⁴-Z⁵—Z⁶—, when present, is independently selected from pyridin-2,4-diyl, pyridin-2,6-diyl, and pyridin-3,5-diyl, unless stated otherwise in any of the embodiment above.

In a twenty-sixth embodiment of the twelfth and thirteenth aspects and first to twenty-fifth, phenylene of —Z¹-Z²—Z³—Z⁴-Z⁵—Z⁶—, when present, is independently selected from 1,3-phenylene and 1,4-phenylene unless stated otherwise in any of the embodiment above.

In a twenty-seventh embodiment of the twelfth and thirteenth aspects and first to twenty-sixth, heterocyclylene, bridged heterocyclylene, and spiro heterocyclylene, of —Z¹-Z²—Z³—Z⁴-Z⁵—Z⁶—, when present, are independently selected from

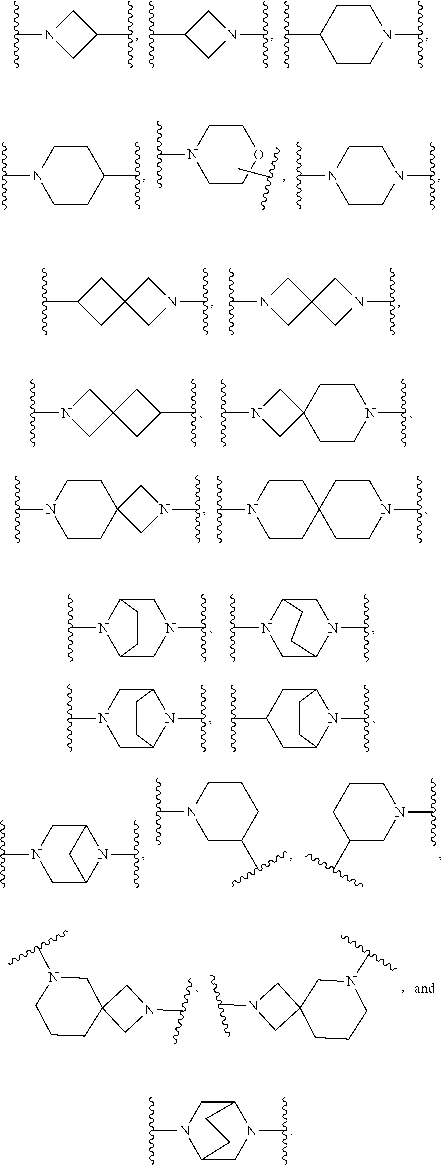

In a twenty-eighth embodiment of the twelfth and thirteenth aspects, -L- is selected from:

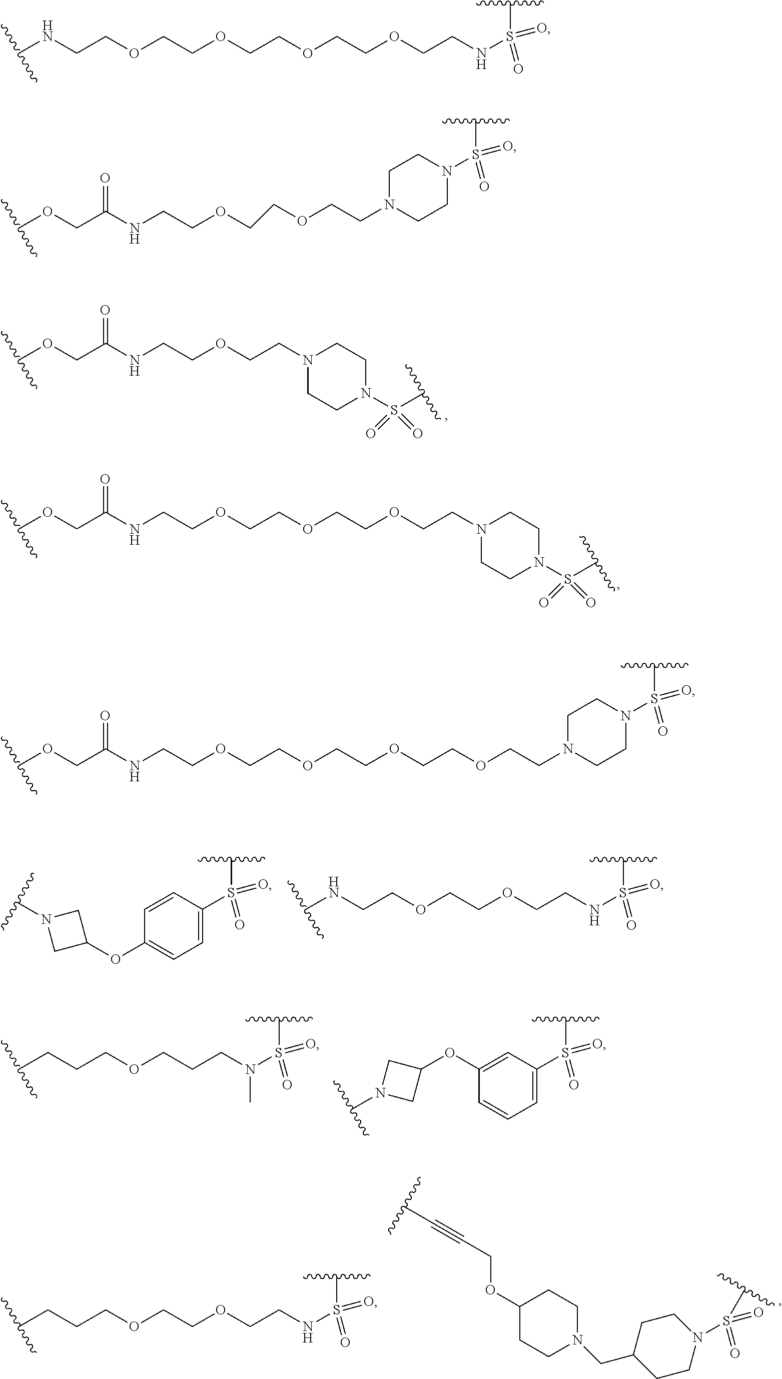

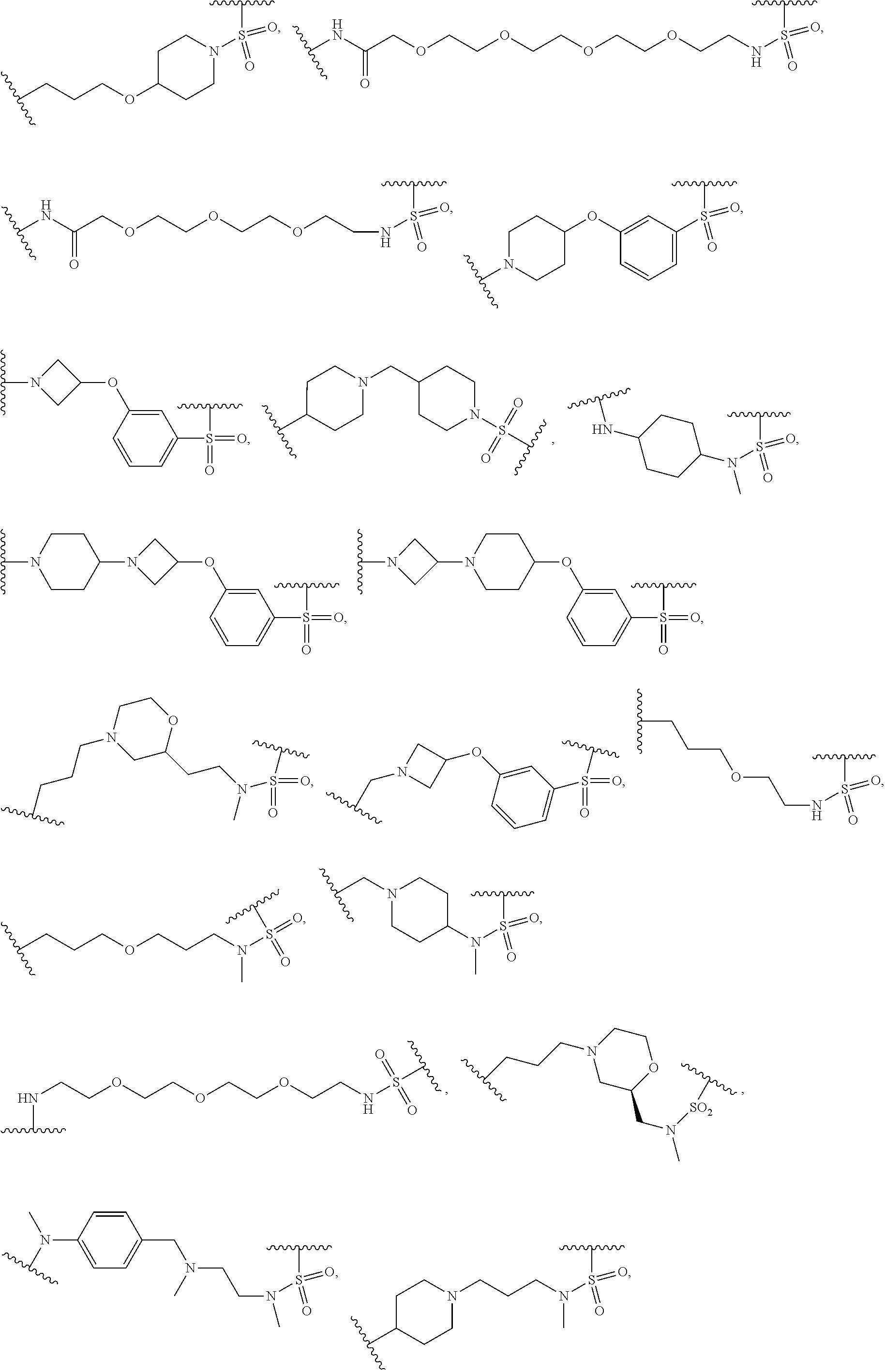

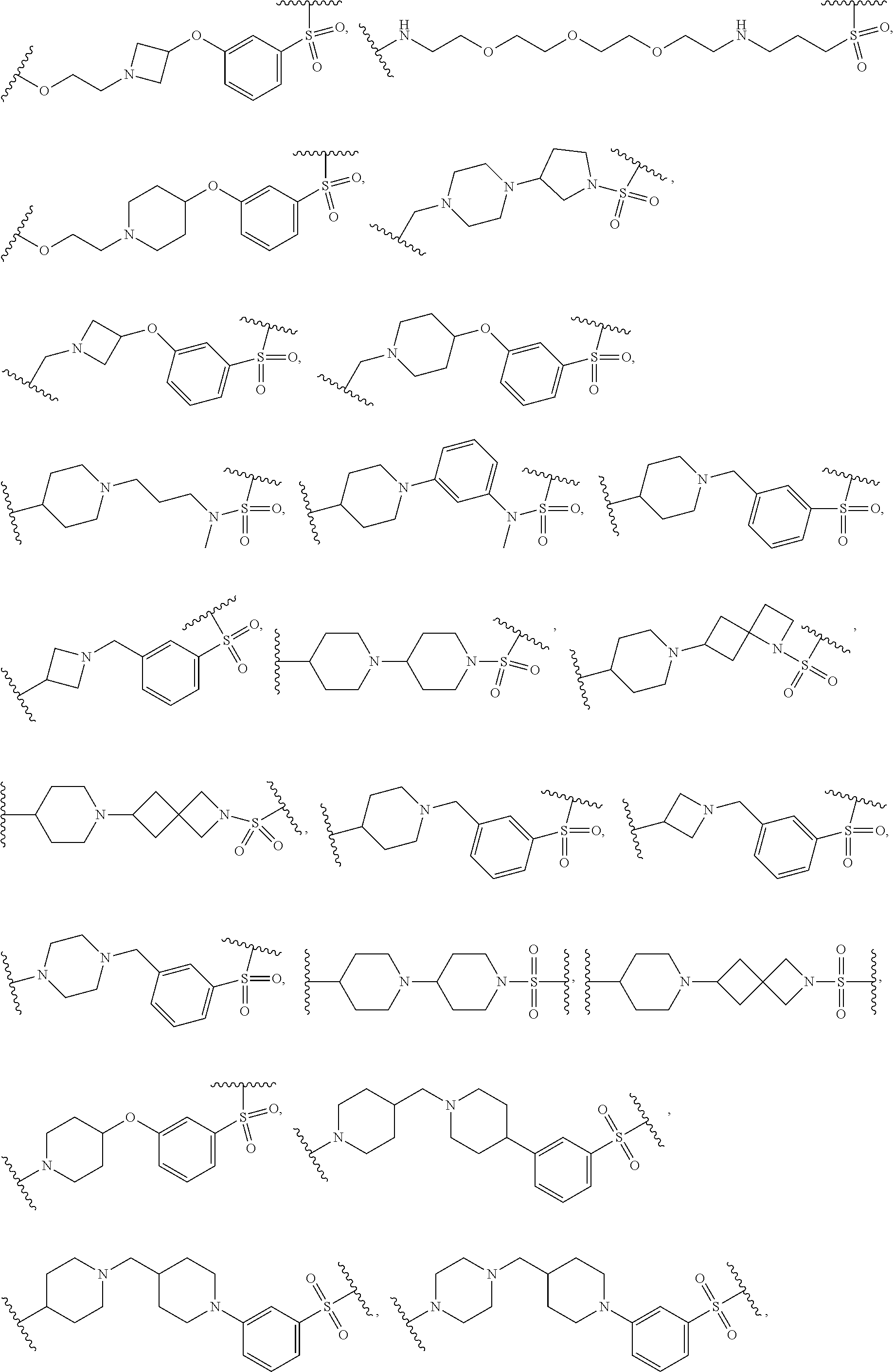

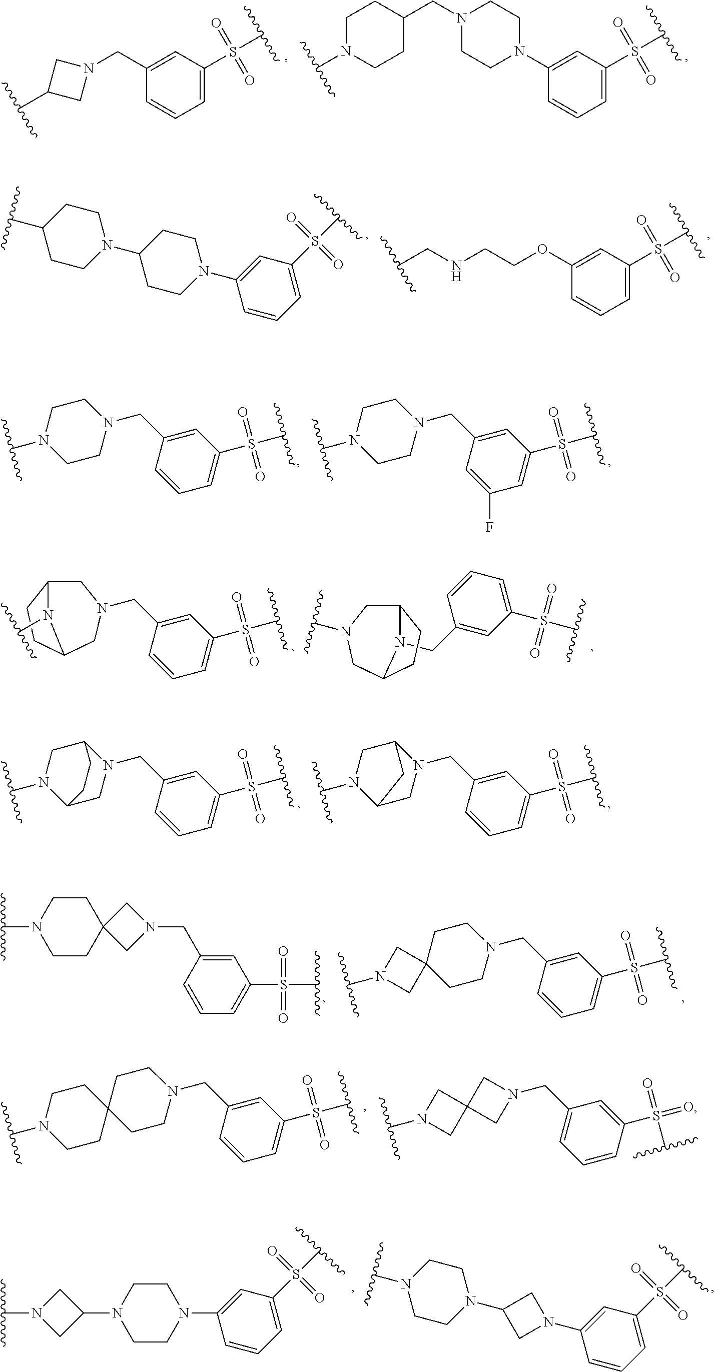

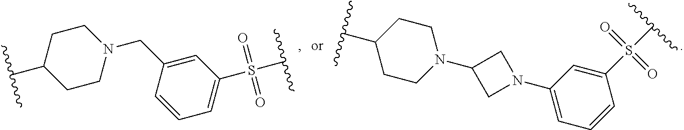 [SMILES: *C1CCN(Cc2cccc(S(*)(=O)=O)c2)CC1 ; *C1CCN(C2CN(c3cccc(S(*)(=O)=O)c3)C2)CC1]

In a twenty-ninth embodiment of the twelfth and thirteenth aspects, -L- is selected from:

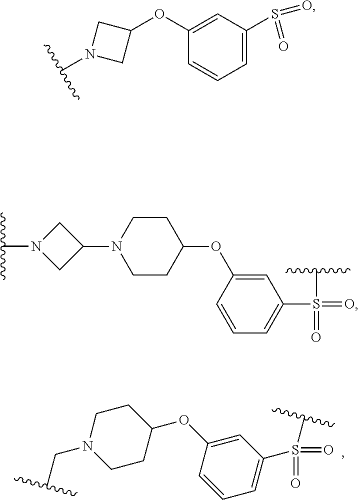

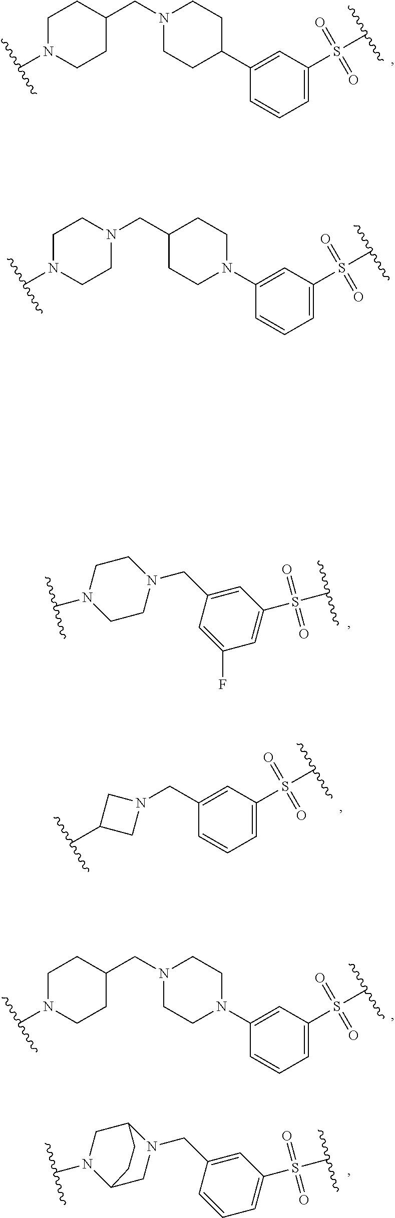 [SMILES: *N1CCC(CN2CCC(c3cccc(S(*)(=O)=O)c3)CC2)CC1 ; *N1CCN(CC2CCN(c3cccc(S(*)(=O)=O)c3)CC2)CC1 ; *N1CCN(Cc2cc(F)cc(S(*)(=O)=O)c2)CC1 ; *C1CN(Cc2cccc(S(*)(=O)=O)c2)C1 ; *N1CCC(CN2CCN(c3cccc(S(*)(=O)=O)c3)CC2)CC1 ; *N1CC2CCC1CN2Cc1cccc(S(*)(=O)=O)c1]

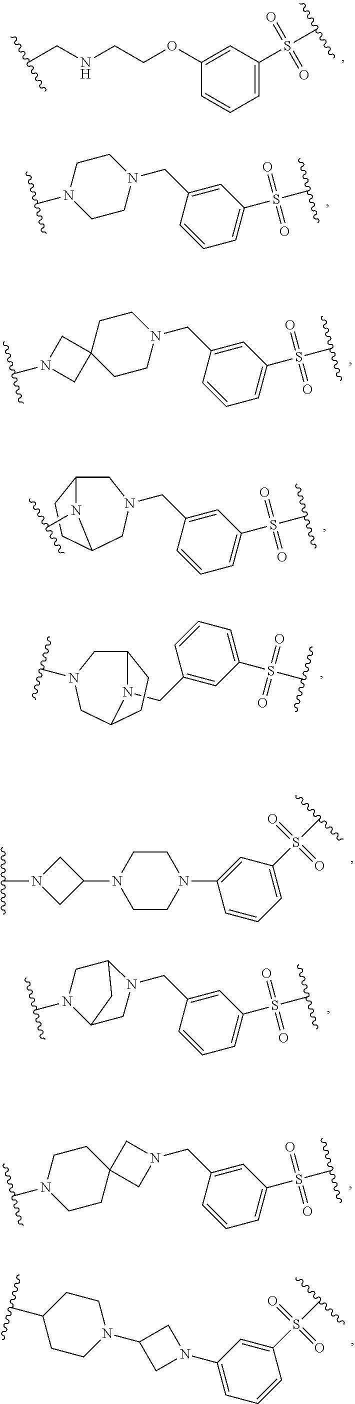

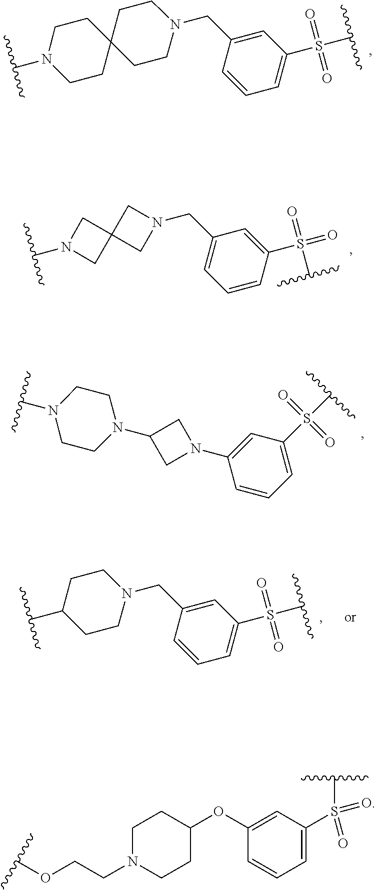 [SMILES: *N1CCC2(CC1)CCN(Cc1cccc(S(*)(=O)=O)c1)CC2 ; *N1CC2(C1)CN(Cc1cccc(S(*)(=O)=O)c1)C2 ; *N1CCN(C2CN(c3cccc(S(*)(=O)=O)c3)C2)CC1 ; *C1CCN(Cc2cccc(S(*)(=O)=O)c2)CC1 ; *OCCN1CCC(Oc2cccc(S(*)(=O)=O)c2)CC1]

In a thirteenth embodiment of the twelfth and thirteenth aspects and first to twenty-eighth embodiments thereof, the CDK2 inhibitor is any one of compound of:
(A) a compound of Formula (VI):

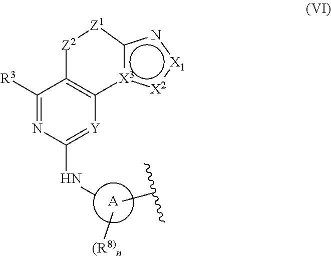

where Formula (VI) corresponds to Formula (I) in PCT Application publication No. WO2020223469A1; and where Y, Z¹, Z², X¹, X², and X³, n, R³ and R⁸ of Formula (VI) and embodiments thereof are as disclosed at page 7 to page 68, line 15, and definitions of terms used in Formula (VI) and the embodiments are as disclosed at page 68, line 16 to page 79, line 15, in PCT Application publication No. WO WO2020223469A1. The formula of Compound (I) and embodiments thereof and paragraphs recited above are incorporated herein by reference in their entireties. The specific compounds disclosed in synthetic examples 1 to 32 of PCT Application publication No. WO WO2020223469A1, are also incorporated herein by reference in their entireties. The bond

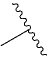

in ring A of Compound (VI) denotes that Compound (VI) is to —Z⁶— of linker L via ring A;
(B) a compound of Formula (VII):

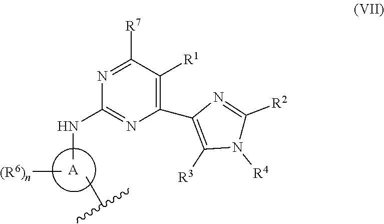

where Formula (VII) corresponds to Formula (I) in PCT Application publication No. WO2021030537A1; and where n, R¹, R², R³, R⁴, R⁶, and R⁷ of Formula (VI) and embodiments thereof (including compounds of formulae (II), (IIa), (III), (IV), (Iva) and (V)) are as disclosed on page 7 to page 51, line 17 and definitions of terms used in Formula (VI) and the embodiments thereof are as disclosed on page 51, line 18 to page 62, in PCT Application publication No. WO2021030537A1. The formulae of Compound (I) and embodiments thereof and paragraphs recited above are incorporated herein by reference in their entireties. The specific compounds disclosed in synthetic examples 1 to 472 of PCT Application publication No. WO2021030537A1, are also incorporated herein by reference in their entireties. The bond

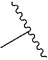

in ring A of Compound (VII) denotes that Compound (VI) is to —Z⁶— of linker L via ring A; or
(C) a compound of Formula (VIII):

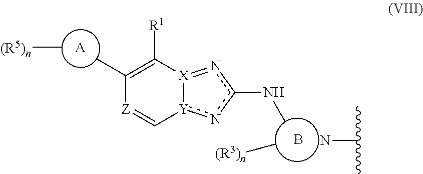

where Formula (VIII) corresponds to Formula (I) in PCT Application publication No. WO2021072232A1; and where n, R¹, R³, R⁵, X, Y, and Z of Formula (VIII) and embodiments thereof (including compounds of formulae (II)—(IXc)) are as disclosed on page 3, line 21 to page 67, line 21, and definitions of terms used in Formula (VIII) and the embodiments thereof are as disclosed on page 68, line 15 to page 78, line 10, in PCT Application publication No. WO2021072232A1. The formulae of Compound (I) and embodiments thereof and paragraphs recited above are incorporated herein by reference in their entireties. The specific compounds disclosed in synthetic examples 1 to 142 of PCT Application publication No. WO2021072232A1, are also incorporated herein by reference in their entireties. The bond

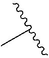

in ring B of Compound (VIII) denotes that Compound (VIII) is connected to —Z⁶— of linker L via ring B.

### General Synthetic Scheme

Compounds Formula (IA′) can be made by the methods depicted in the reaction schemes shown below.

The starting materials and reagents used in preparing these compounds are either available from commercial suppliers such as Aldrich Chemical Co., (Milwaukee, Wis.), Bachem (Torrance, Calif.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4th Edition) and Larock's Comprehensive Organic Transformations (VCH Publishers Inc., 1989). These schemes are merely illustrative of some methods by which the compounds Formula (I) can be synthesized, and various modifications to these schemes can be made and will be suggested to one skilled in the art reading this disclosure. The starting materials and the intermediates, and the final products of the reaction may be isolated and purified if desired using conventional techniques, including but not limited to filtration, distillation, crystallization, chromatography and the like. Such materials may be characterized using conventional means, including physical constants and spectral data.

Unless specified to the contrary, the reactions described herein take place at atmospheric pressure over a temperature range from about −78° C. to about 150° C., such as from about 0° C. to about 125° C. and further such as at about room (or ambient) temperature, e.g., about 20° C.

Compounds of Formula (IA′) where Degron is an E3 ligase ligand of formula (i) where ring A is a group of formula (a), (b), or (c) where X¹, X², and X³ are —CONH—, L is attached to Hy via —NH— and other groups are as defined in the Summary can be prepared as described in Scheme 1 below

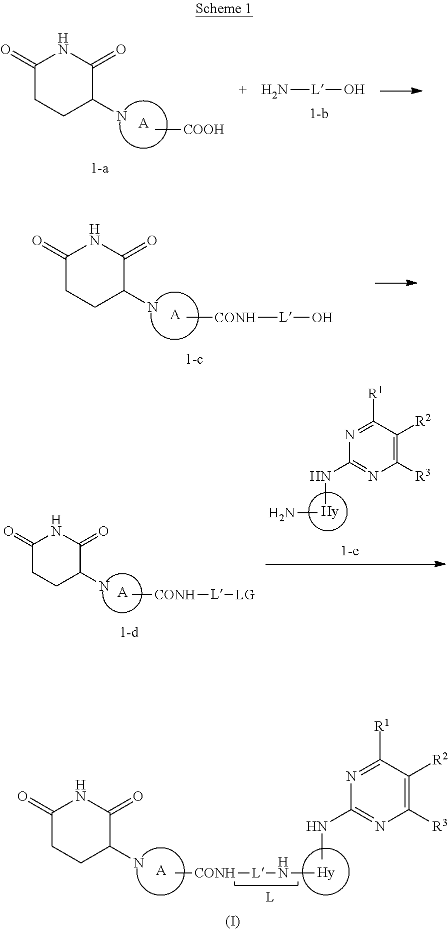

Coupling of carboxylic acid group in 1-a and a compound of formula 1-b where L′ is precursor group to L in the compound of Formula (I) as defined in the Summary, with a suitable coupling reagent such as HATU provides the corresponding amide of formula 1-c. The hydroxy group in 1-c is can be converted into a leaving group such as mesylate, triflate, and the like, by methods well known in the art to provide a compound of 1-d. Reaction of compound 1-d with a compound of formula 1-e in presence of a suitable base under conditions known in the art provides a compound of Formula (I) where X, X¹, X², and X³ are —CONH— and L is attached to Hy via —NH—.

Compounds of Formula (IA′) where Degron is an E3 ligase ligand of formula (i) where ring A is a group of formula (a), (b), or (c) where X¹, X², and X³ are —NH—, L is attached to Hy via —O— and other groups are as defined in the Summary can be prepared as described in Scheme 2 below

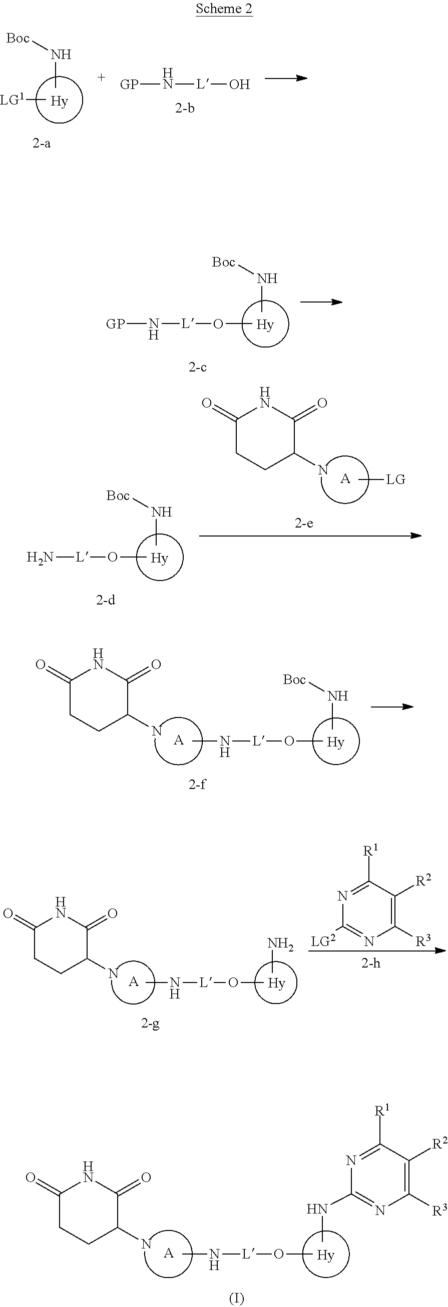

Displacement of a LG¹ (leaving group) such as halo, methylsulfonyl, and the like, in compound of formula 2-a by an alcohol compound of formula 2-b where PG is an a suitable amino protecting group such as benzyl or CBz and L′ is a precursor group to L as defined in the Summary, in presence of a suitable base such as NaH, and the like provides a compound of formula 2-c where -L′- is as precursor group of L in the compound of Formula (I) as defined in the Summary. Removal of the protecting group under suitable conditions provides an amine compound of formula 2-d. Displacement of a leaving group (LG) such as halide, sulfonate, and the like in a compound of formula 2-e where ring A is as defined in the Summary, by the amino group of formula 2-d provides a compound of formula 2-f. Removal of the Boc protecting group in compound 2-f using an acid like TFA provides a compound of formula 2-g. Reaction of compound 2-g with a compound of formula 2-h where LG² is a suitable leaving group such as Cl or SO₂Me and R¹, R² and R³ are as defined in the Summary under suitable conditions such as acidic, basic or transition metal catalyzed reaction conditions well known in the art, provides a compound of Formula (I).

Compounds of Formula (IA) where X¹, X², and X³ are ethylene, L is attached to Hy via —NH— and other groups are as defined in the Summary can be prepared as described in Scheme 3 below:

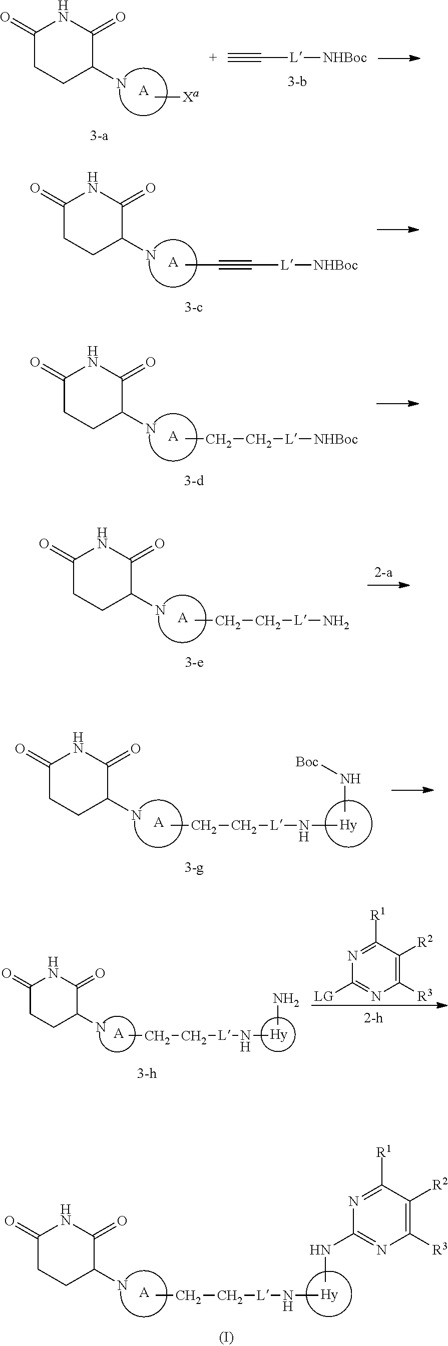

Sonogashira coupling of a compound of formula 3-a Xa is a halo with terminal alkyne group of a compound of formula 3-b where L′ is a precursor group of L in the compound of Formula (1) as defined in the Summary, in the presence of a palladium(O) catalyst, a copper(I) cocatalyst and a suitable base such as triethylamine, and the like provides a compound of formula 3-c. Hydrogenated of the triple bond provides a corresponding compound of formula 3-d. Removal of the Boc group in 3-d using an acid such as TFA provides an amine compound of formula 3-e, Reaction of 3-e with a compound of formula 2-a under suitable conditions provides a compound of formula 3-g. Removal of the Boc group in compound 3-g, followed by reaction of the resulting amine of formula 3-h with a compound of formula 2-h as described in above provides a compound of Formula (I).

Compounds of formula 2-h having the structure

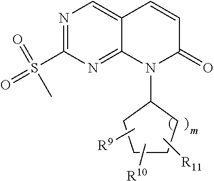

where R¹, R⁷ and R⁸ are hydrogen, LG is —SO₂Me, and m, R⁹, R¹⁰, and R¹¹ are as defined in Summary can be synthesized by method illustrated in Method (a) below.

### Method (a):

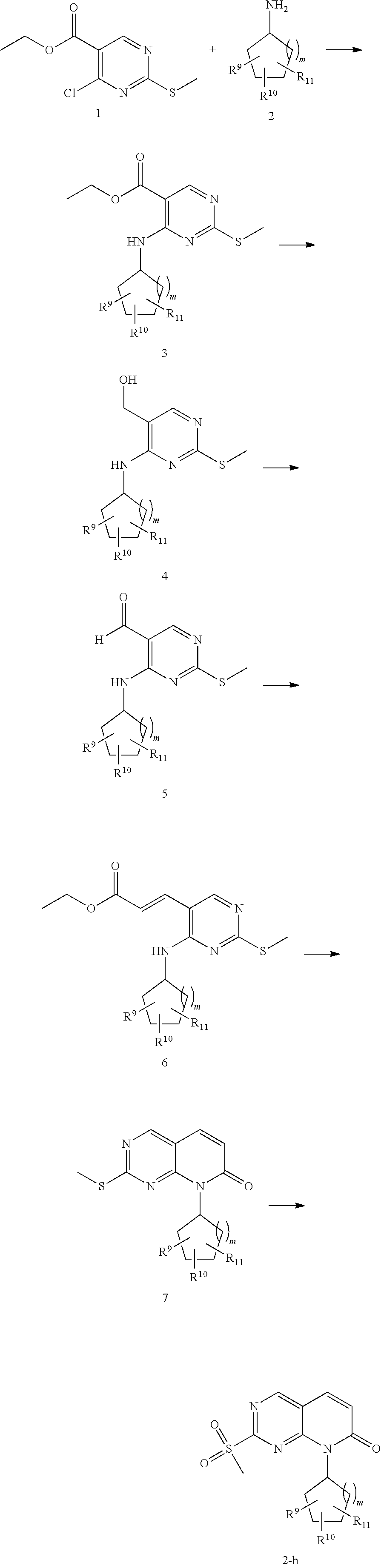

Displacement of the chloride in compound 1 with an amine of formula 2 under basic condition as such TEA provides a compound of formula 3. Compound 2 and amine 3 are commercially available or they can be prepared by methods well known the art. For example, cyclopentanamine is commercially available.

Reduction of the ester group in 3 with a suitable reducing reagent such LiAlH₄ provides a hydroxy compound of formula 4, which can be converted to corresponding aldehyde of formula 5 with an oxidizing agent such as MnO₂. Olefination of 5 with ethyl 2-(triphenyl-k-phosphanylidene)acetate provides a compound of formula 6, which can undergo cyclization with R⁴HH₂ in the presence of a base such as DBU under heating condition to provide a compound of formula 7. Compound 7 can then be converted to a compound of formula 2-h where LG is SO₂Me by treating with an oxidant such as m-CPBA.

Preparation of compounds of Formula (IA′) where ring A and L are various other groups are disclosed in Synthetic Examples below.

### Utility

The compound of Formula (IA′), (IA) and (I) could cause degradation of CDK2 protein and hence are useful in the treatment of diseases mediated by CDK2. Increasing evidence suggests that overactivated CDK2 leads to abnormal cell cycle regulation and proliferation in cancer cells. While CDK2 mutations are rarely found, the kinase activity of CDK2/Cyclin E or CDK2/Cyclin A complexes is elevated via several mechanisms in human cancers. Cyclin E has been found to be frequently amplified in human malignancies, for example, in ovarian cancer and breast cancer. In some cancer types loss-of-function mutations in FBXW7, a component of SCF^{Fbw7} ubiquitin E3 ligase responsible for cyclin E degradation, also leads to cyclin E overexpression and CDK2 activation. Alternatively, certain cancer cells express a hyperactive, truncated form of cyclin E. In addition, cyclin A amplification and overexpression have also been reported in various cancers such as hepatocellular carcinomas, colorectal and breast cancers. In some tumors, catalytic activity of CDK2 is increased following loss of the expression or alteration of the location of the endogenous CDK2 inhibitor p27 or p21. In addition, CDC25A and CDC25B, protein phosphatases responsible for the dephosphorylations that activate the CDK2, are overexpressed in various tumors. These various mechanisms of CDK2 activation have been validated using mouse cancer models. Furthermore, CDK2/cyclin E phosphorylates oncogenic Myc to oppose ras-induced senescence, highlighting the importance of CDK2 in myc/ras-induced tumorigenesis. Inactivation of CDK2 has been shown to be synthetically lethal to myc over-expressing cancer cells. Therefore, a compound of the invention may be particularly useful for treating tumors characterized by 1) overexpression of CDK2, 2) amplification of cyclin E or cyclin A. 3) loss-of-function of mutation in FBXW7, 4) expression of truncated cyclin E, 5) dysregulation of p21 or p27, and 6) hyperactive MYC/RAS.

CDK2 activation as a result of cyclin E amplification or overexpression has also been identified as a key primary or acquired resistance pathway to tumors treated by CDK4/6 inhibitors or trastuzumab.

In some embodiments, the cancer is hepatocellular carcinomas, colorectal and breast cancers. In some embodiments, the cancer is ovarian cancer. In some such embodiments, the ovarian cancer is characterized by amplification or overexpression of CCNE1 and/or CCNE2. In other embodiments, the cancer is breast cancer, including, e.g., ER-positive/HR-positive breast cancer, HER2-negative breast cancer; ER-positive/HR-positive breast cancer, HER2-positive breast cancer; triple negative breast cancer (TNBC); or inflammatory breast cancer. In some embodiments, the breast cancer is endocrine resistant breast cancer, trastuzumab resistant breast cancer, or breast cancer demonstrating primary or acquired resistance to CDK4/CDK6 inhibition. In some embodiments, the breast cancer is advanced or metastatic breast cancer. In some embodiments of each of the foregoing, the breast cancer is characterized by amplification or overexpression of CCNE1 and/or CCNE2.

### Testing

CDK2 potency and CDK2 degradation activities of the compounds of the present disclosure can be tested using the in vitro assays described in Biological Examples below.

### Pharmaceutical Compositions

In general, the compounds Formula (IA′), (IA), or (I) (unless stated otherwise, reference to compound/compounds of Formula (IA) or (I) herein includes any embodiments thereof described herein or a pharmaceutically acceptable salt thereof) will be administered in a therapeutically effective amount by any of the accepted modes of administration for agents that serve similar utilities. Therapeutically effective amounts of compounds Formula (IA′), (IA), or (I) may range from about 0.01 to about 500 mg per kg patient body weight per day, which can be administered in single or multiple doses. A suitable dosage level may be from about 0.1 to about 250 mg/kg per day; about 0.5 to about 100 mg/kg per day. A suitable dosage level may be about 0.01 to about 250 mg/kg per day, about 0.05 to about 100 mg/kg per day, or about 0.1 to about 50 mg/kg per day. Within this range the dosage can be about 0.05 to about 0.5, about 0.5 to about 5 or about 5 to about 50 mg/kg per day. For oral administration, the compositions can be provided in the form of tablets containing about 1.0 to about 1000 milligrams of the active ingredient, particularly about 1, 5, 10, 15, 20, 25, 50, 75, 100, 150, 200, 250, 300, 400, 500, 600, 750, 800, 900, and 1000 milligrams of the active ingredient. The actual amount of the compound Formula (IA′), (IA), or (I), i.e., the active ingredient, will depend upon numerous factors such as the severity of the disease to be treated, the age and relative health of the patient, the potency of the compound being utilized, the route and form of administration, and other factors.

In general, compounds Formula (IA′), (IA), or (I) will be administered as pharmaceutical compositions by any one of the following routes: oral, systemic (e.g., transdermal, intranasal or by suppository), or parenteral (e.g., intramuscular, intravenous or subcutaneous) administration. The preferred manner of administration is oral using a convenient daily dosage regimen, which can be adjusted according to the degree of affliction. Compositions can take the form of tablets, pills, capsules, semisolids, powders, sustained release formulations, solutions, suspensions, elixirs, aerosols, or any other appropriate compositions.

The choice of formulation depends on various factors such as the mode of drug administration (e.g., for oral administration, formulations in the form of tablets, pills or capsules, including enteric coated or delayed release tablets, pills or capsules are preferred) and the bioavailability of the drug substance.

The compositions are comprised of in general, a compound of Formula (IA′), (IA), or (I) in combination with at least one pharmaceutically acceptable excipient. Acceptable excipients are generally non-toxic, aid administration, and do not adversely affect the therapeutic benefit of the compound of Formula (IA′), (IA), or (I). Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

The compounds of Formula (IA′), (IA), or (I) may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds of Formula (IA′), (IA), or (I) may also be formulated as a depot preparation. Such long-acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds of Formula (IA′), (IA), or (I) may also be formulated in rectal compositions such as suppositories or retention enemas, e.g., containing conventional suppository bases such as cocoa butter, polyethylene glycol, or other glycerides.

Certain compounds of Formula (IA′), (IA), or (I) may be administered topically, that is by non-systemic administration. This includes the application of a compound of Formula (IA′), (IA), or (I) externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient for topical administration may comprise, for example, from 0.001% to 10% w/w (by weight) of the formulation. In certain embodiments, the active ingredient may comprise as much as 10% w/w. In other embodiments, it may comprise less than 5% w/w. In certain embodiments, the active ingredient may comprise from 2% w/w to 5% w/w. In other embodiments, it may comprise from 0.1% to 1% w/w of the formulation.

For administration by inhalation, compounds of Formula (IA′), (IA), or (I) may be conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds of Formula (IA′), (IA), or (I) may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator. Other suitable pharmaceutical excipients and their formulations are described in Remington's Pharmaceutical Sciences, edited by E. W. Martin (Mack Publishing Company, 20th ed., 2000).

The level of the compound of Formula (IA′), (IA), or (I) in a formulation can vary within the full range employed by those skilled in the art. Typically, the formulation will contain, on a weight percent (wt. %) basis, from about 0.01-99.99 wt. % of a compound of Formula (IA′), (IA), or (I) based on the total formulation, with the balance being one or more suitable pharmaceutical excipients. For example, the compound is present at a level of about 1-80 wt. %.

### Combinations and Combination Therapies

The compounds of Formula (IA′), (IA), or (I) may be used in combination with one or more other drugs in the treatment of diseases or conditions for which compounds of Formula (IA′), (IA), or (I) or the other drugs may have utility. Such other drug(s) may be administered, by a route and in an amount commonly used therefore, contemporaneously or sequentially with a compound of Formula (IA′), (IA), or (I). When a compound of Formula (IA′), (IA), or (I) is used contemporaneously with one or more other drugs, a pharmaceutical composition in unit dosage form containing such other drugs and the compound of Formula (IA′), (IA), or (I) is preferred. However, the combination therapy may also include therapies in which the compound of Formula (IA′), (IA), or (I) and one or more other drugs are administered on different overlapping schedules. It is also contemplated that when used in combination with one or more other active ingredients, the compounds of Formula (IA′), (IA), or (I) and the other active ingredients may be used in lower doses than when each is used singly.

Accordingly, the pharmaceutical compositions of the present disclosure also include those that contain one or more other drugs, in addition to a compound of Formula (IA′), (IA), or (I).

The above combinations include combinations of a compound of Formula (IA′), (IA), or (I) not only with one other drug, but also with two or more other active drugs. Likewise, a compound of Formula (IA′), (IA), or (I) may be used in combination with other drugs that are used in the prevention, treatment, control, amelioration, or reduction of risk of the diseases or conditions for which a compound of Formula (IA′), (IA), or (I) is useful. Such other drugs may be administered, by a route and in an amount commonly used therefore, contemporaneously or sequentially with a compound of Formula (IA′), (IA), or (I). When a compound of Formula (IA′), (IA), or (I) is used contemporaneously with one or more other drugs, a pharmaceutical composition containing such other drugs in addition to the compound of Formula (IA′), (IA), or (I) can be used. Accordingly, the pharmaceutical compositions of the present disclosure also include those that also contain one or more other active ingredients, in addition to a compound of Formula (IA′), (IA), or (I). The weight ratio of the compound of this disclosure to the second active ingredient may be varied and will depend upon the effective dose of each ingredient. Generally, an effective dose of each will be used.

Where the subject in need is suffering from or at risk of suffering from cancer, the subject can be treated with a compound of Formula (IA′), (IA), or (I) in any combination with one or more other anti-cancer agents including but not limited to: MAP kinase pathway (RAS/RAF/MEK/ERK) inhibitors including but not limited to: Vemurafanib (PLX4032), Dabrafenib, Encorafenib (LGX818), TQ-B3233, XL-518 (Cas No. 1029872-29-4, available from ACC Corp); trametinib, selumetinib (AZD6244), TQ-B3234, PD184352, PD325901, TAK-733, pimasertinib, binimetinib, refametinib, cobimetinib (GDC-0973), AZD8330, BVD-523, LTT462, Ulixertinib, AMG510, ARS853, and any RAS inhibitors disclosed in patents WO2016049565, WO2016164675, WO2016168540, WO2017015562, WO2017058728, WO2017058768, WO2017058792, WO2017058805, WO2017058807, WO2017058902, WO2017058915, WO2017070256, WO2017087528, WO2017100546, WO2017172979, WO2017201161, WO2018064510, WO2018068017, WO2018119183;
CSF1R inhibitors (PLX3397, LY3022855, etc.) and CSF1R antibodies (IMC-054, RG7155) TGF beta receptor kinase inhibitor such as LY2157299;
BTK inhibitor such as ibrutinib; BCR-ABL inhibitors: Imatinib (Gleevec®); Inilotinib hydrochloride; Nilotinib (Tasigna®); Dasatinib (BMS-345825); Bosutinib (SKI-606); Ponatinib (AP24534); Bafetinib (INNO406); Danusertib (PHA-739358), AT9283 (CAS 1133385-83-7); Saracatinib (AZD0530); and N-[2-[(1S,4R)-6-[[4-cyclobutylarmno)-5-(trifluoromethyl)-2-pyrimidinyl]amino]-1,2,3,4-tetrahydronaphthalen-1,4-imin-9-yl]-2-oxoethyl]-acetamide (PF-03814735, CAS 942487-16-3);
ALK inhibitors: PF-2341066 (XALKOPJ®; crizotinib); 5-chloro-N4-(2-(isopropyl-sulfonyl)phenyl)-N2-(2-methoxy-4-(4-(4-methylpiper azin-1-yl)piperidin-1-yl)phenyl)pyrimidine-2,4-diamine; GSK1838705 A; CH5424802; Ceritinib (ZYKADIA); TQ-B3139, TQ-B3101 PI3K inhibitors: 4-[2-(1H-indazol-4-yl)-6-[[4-(methylsulfonyl)piperazin-1-yl]methyl]thieno[3,2-d]-pyrimidin-4-yl]morpholine (also known as GDC 0941 and described in PCT Publication Nos. WO 09/036082 and WO 09/055730), 2-methyl-2-[4-[3-methyl-2-oxo-8-(quinolin-3-yl)-2,3-dihydro-imidazo[4,5-c]quinolin-1-yl]phenyl]propionitrile (also known as BEZ 235 or NVP-BEZ 235, and described in PCT Publication No. WO 06/122806);
Vascular Endothelial Growth Factor (VEGF) receptor inhibitors: Bevacizumab (sold under the trademark Avastin® by Genentech/Roche), axitinib, (N-methyl-2-[[3-[(E)-2-pyridin-2-ylethenyl]-1H-indazol-6-yl]sulfanyl]benzamide, also known as AG013736, and described in PCT Publication No. WO 01/002369), Brivanib Alaninate ((S)-((R)-1-(4-(4-fluoro-2-methyl-1H-indol-5-yloxy)-5-methylpyrrolo[2,1-f][1,2,4]triazin-6-yloxy)propan-2-yl)2-aminopropanoate, also known as BMS-582664), motesanib (N-(2,3-dihydro-3,3-dimethyl-1H-indol-6-yl)-2-[(4-pyridinyl-methyl)amino]-3-pyridinecarboxamide, and described in PCT Publication No. WO 02/066470), pasireotide (also known as SOM230, and described in PCT Publication No. WO 02/010192), sorafenib (sold under the tradename Nexavar®); AL-2846 MET inhibitor such as foretinib, carbozantinib, or crizotinib;
FLT3 inhibitors—sunitinib malate (sold under the tradename Sutent® by Pfizer); PKC412 (midostaurin); tanutinib, sorafenib, lestaurtinib, KW-2449, quizartinib (AC220) and crenolanib;
Epidermal growth factor receptor (EGFR) inhibitors: Gefitnib (sold under the tradename Iressa®), N-[4-[(3-chloro-4-fluorophenyl)amino]-7-[[(3″S″)-tetrahydro-3-furanyl]oxy]-6-quinazolinyl]-4(dimethylamino)-2-butenamide, sold under the tradename Tovok® by Boehringer Ingelheim), cetuximab (sold under the tradename Erbitux® by Bristol-Myers Squibb), panitumumab (sold under the tradename Vectibix® by Amgen);
HER2 receptor inhibitors: Trastuzumab (sold under the trademark Herceptin® by Genentech/Roche), neratinib (also known as HKI-272, (2E)-N-[4-[[3-chloro-4-[(pyridin-2-yl)methoxy]phenyl]amino]-3-cyano-7-ethoxyquinolin-6-yl]-4-(dimethylamino)but-2-enamide, and described PCT Publication No. WO 05/028443), lapatinib or lapatinib ditosylate (sold under the trademark Tykerb® by GlaxoSmithKline); Trastuzumab emtansine (in the United States, ado-trastuzumab emtansine, trade name Kadcyla)—an antibody-drug conjugate consisting of the monoclonal antibody trastuzumab (Herceptin) linked to the cytotoxic agent mertansine (DM1);
HER dimerization inhibitors: Pertuzumab (sold under the trademark Omnitarg®, by Genentech);
CD20 antibodies: Rituximab (sold under the trademarks Riuxan® and MabThera® by Genentech/Roche), tositumomab (sold under the trademarks Bexxar® by GlaxoSmithKline), ofatumumab (sold under the trademark Arzerra® by GlaxoSmithKline);
Tyrosine kinase inhibitors: Erlotinib hydrochloride (sold under the trademark Tarceva® by Genentech/Roche), Linifanib (N-[4-(3-amino-1H-indazol-4-yl)phenyl]-N′-(2-fluoro-5-methylphenyl)urea, also known as ABT 869, available from Genentech), sunitinib malate (sold under the tradename Sutent® by Pfizer), bosutinib (4-[(2,4-dichloro-5-methoxyphenyl)amino]-6-methoxy-7-[3-(4-methylpiperazin-1-yl)propoxy]quinoline-3-carbonitrile, also known as SKI-606, and described in U.S. Pat. No. 6,780,996), dasatinib (sold under the tradename Sprycel® by Bristol-Myers Squibb), armala (also known as pazopanib, sold under the tradename Votrient® by GlaxoSmithKline), imatinib and imatinib mesylate (sold under the tradenames Gilvec® and Gleevec® by Novartis);
DNA Synthesis inhibitors: Capecitabine (sold under the trademark Xeloda® by Roche), gemcitabine hydrochloride (sold under the trademark Gemzar® by Eli Lilly and Company), nelarabine ((2R3S,4R,5R)-2-(2-amino-6-methoxy-purin-9-yl)-5-(hydroxymethyl)oxolane-3,4-diol, sold under the tradenames Arranon® and Atriance® by GlaxoSmithKline);
Antineoplastic agents: oxaliplatin (sold under the tradename Eloxatin® ay Sanofi-Aventis and described in U.S. Pat. No. 4,169,846);
Human Granulocyte colony-stimulating factor (G-CSF) modulators: Filgrastim (sold under the tradename Neupogen® by Amgen);
Immunomodulators: Afutuzumab (available from Roche®), pegfilgrastim (sold under the tradename Neulasta® by Amgen), lenalidomide (also known as CC-5013, sold under the tradename Reylimid®), thalidomide (sold under the tradename Thalomid®);
CD40 inhibitors: Dacetuzumab (also known as SGN-40 or huS2C6, available from Seattle Genetics, Inc); Pro-apoptotic receptor agonists (PARAs): Dulanermin (also known as AMG-951, available from Amgen/Genentech);
Hedgehog antagonists: 2-chloro-N-[4-chloro-3-(2-pyridinyl)phenyl]-4-(methylsulfonyl)-benzamide (also known as GDC-0449, and described in PCT Publication No. WO 06/028958);
Phospholipase A2 inhibitors: Anagrelide (sold under the tradename Agrylin®);
BCL-2 inhibitors: 4-[4-[[2-(4-chlorophenyl)-5,5-dimethyl-1-cyclohexen-1-yl]methyl]-1-piperazinyl]-N-[[4-[[(1R)-3-(4-morpholinyl)-1-[(phenylthio)methyl]propyl]amino]-3-[(trifluoromethyl)sulfonyl]phenyl]sulfonyl]benzamide (also known as ABT-263 and described in PCT Publication No. WO 09/155386);
MCl-1 inhibitors: MIK665, S64315, AMG 397, and AZD5991;
Aromatase inhibitors: Exemestane (sold under the trademark Aromasin® by Pfizer), letrozole (sold under the tradename Femara® by Novartis), anastrozole (sold under the tradename Arimidex®);
Topoisomerase I inhibitors: Irinotecan (sold under the trademark Camptosar® by Pfizer), topotecan hydrochloride (sold under the tradename Hycamtin® by GlaxoSmithKline);
Topoisomerase II inhibitors: etoposide (also known as VP-16 and Etoposide phosphate, sold under the tradenames Toposar®, VePesid® and Etopophos®), teniposide (also known as VM-26, sold under the tradename Vumon®);
mTOR inhibitors: Temsirolimus (sold under the tradename Torisel® by Pfizer), ridaforolimus (formally known as deferolimus, (1R,2R,4S)-4-[(2R)-2[(1R,9S,12S,15R,16E,18R,19R,21R,23S,24E,26E,28Z,30S,32S,35R)-1,18-dihydroxy-19,30-dimethoxy-15, 17, 21, 23, 29,35-hexamethyl-2,3,10,14,20-pentaoxo-11,36-dioxa-4-azatricyclo[30.3.1.0 4′ 9]hexatriaconta-16,24,26,28-tetraen-12-yl]propyl]-2-methoxycyclohexyl dimethylphosphinate, also known as AP23573 and MK8669, and described in PCT Publication No. WO 03/064383), everolimus (sold under the tradename Afinitor® by Novartis);
Proteasome inhibitor such as carfilzomib, MLN9708, delanzomib, or bortezomib;
BET inhibitors such as INCB054329, OTX015, and CPI-0610;
LSD1 inhibitors such as GSK2979552, and INCB059872;
HIF-2α inhibitors such as PT2977 and PT2385;
Osteoclastic bone resorption inhibitors: 1-Hydroxy-2-imidazol-1-yl-phosphonoethyl) phosphonic acid monohydrate (sold under the tradename Zometa® by Novartis); CD33 Antibody Drug Conjugates: Gemtuzumab ozogamicin (sold under the tradename Mylotarg® by Pfizer/Wyeth);
CD22 Antibody Drug Conjugates: Inotuzumab ozogamicin (also referred to as CMC-544 and WAY-207294, available from Hangzhou Sage Chemical Co., Ltd.);
CD20 Antibody Drug Conjugates: Ibritumomab tiuxetan (sold under the tradename Zevalin®);
Somatostain analogs: octreotide (also known as octreotide acetate, sold under the tradenames Sandostatin® and Sandostatin LAR®);
Synthetic Interleukin-11 (IL-11): oprelvekin (sold under the tradename Neumega® by Pfizer/Wyeth);
Synthetic erythropoietin: Darbepoetin alfa (sold under the tradename Aranesp® by Amgen);
Receptor Activator for Nuclear Factor κ B (RANK) inhibitors: Denosumab (sold under the tradename Prolia® by Amgen);
Thrombopoietin mimetic peptibodies: Romiplostim (sold under the tradename Nplate® by Amgen);
Cell growth stimulators: Palifermin (sold under the tradename Kepivance® by Amgen);
Anti-Insulin-like Growth Factor-1 receptor (IGF-1R) antibodies: Figitumumab (also known as CP-751,871, available from ACC Corp), robatumumab (CAS No. 934235-44-6);
Anti-CSI antibodies: Elotuzumab (HuLuc63, CAS No. 915296-00-3);
CD52 antibodies: Alemtuzumab (sold under the tradename Campath®);
Histone deacetylase inhibitors (HDI): Voninostat (sold under the tradename Zolinza® by Merck);
Alkylating agents: Temozolomide (sold under the tradenames Temodar® and Temodal® by Schering-Plough/Merck), dactinomycin (also known as actinomycin-D and sold under the tradename Cosmegen®), melphalan (also known as L-PAM, L-sarcolysin, and phenylalanine mustard, sold under the tradename Alkeran®), altretamine (also known as hexamethylmelamine (HMM), sold under the tradename Hexalen®), carmustine (sold under the tradename BiCNU®), bendamustine (sold under the tradename Treanda®), busulfan (sold under the tradenames Busulfex® and Myleran®), carboplatin (sold under the tradename Paraplatin®), lomustine (also known as CCNU, sold under the tradename CeeNU®), cisplatin (also known as CDDP, sold under the tradenames Platinol® and Platinol®-AQ), chlorambucil (sold under the tradename Leukeran®), cyclophosphamide (sold under the tradenames Cytoxan® and Neosar), dacarbazine (also known as DTIC, DIC and imidazole carboxamide, sold under the tradename DTIC-Dome®), altretamine (also known as hexamethylmelamine (HMM) sold under the tradename Hexalen®), ifosfamide (sold under the tradename Ifex®), procarbazine (sold under the tradename Matulane®), mechlorethamine (also known as nitrogen mustard, mustine and mechloroethamine hydrochloride, sold under the tradename Mustargen®), streptozocin (sold under the tradename Zanosar®), thiotepa (also known as thiophosphoamide, TESPA and TSPA, sold under the tradename Thioplex®; Biologic response modifiers: bacillus calmette-guerin (sold under the tradenames theraCys® and TICE® BCG), denileukin diftitox (sold under the tradename Ontak®);
Anti-tumor antibiotics: doxorubicin (sold under the tradenames Adriamycin® and Rubex®), bleomycin (sold under the tradename lenoxane), daunorubicin (also known as dauorubicin hydrochloride, daunomycin, and rubidomycin hydrochloride, sold under the tradename Cerubidine®), daunorubicin liposomal (daunorubicin citrate liposome, sold under the tradename DaunoXome), mitoxantrone (also known as DHAD, sold under the tradename Novantronef), epirubicin (sold under the tradename Ellence™), idarubicin (sold under the tradenames Idamycin®, Idamycin PFS®), mitomycin C (sold under the tradename Mutamycin®);
Anti-microtubule agents: Estramustine (sold under the tradename Emcyl®);
Cathepsin K inhibitors: Odanacatib (also known as MK-0822, N-(1-cyanocyclopropyl)-4-fluoro-N-2-((1S)-2,2,2-trifluoro-1-[4′-(methylsulfonyl)biphenyl-4-yl]ethyl)-L-leucinamide, available from Lanzhou Chon Chemicals, ACC Corp., and ChemieTek, and described in PCT Publication no. WO 03/075836); Epothilone B analogs: Ixabepilone (sold under the tradename Lxempra® by Bristol-Myers Squibb);
Heat Shock Protein (HSP) inhibitors: Tanespimycin (17-allylamino-17-demethoxy-geldanamycin, also known as KOS-953 and 17-AAG, available from SIGMA, and described in U.S. Pat. No. 4,261,989), NVP—HSP990, AUY922, AT13387, STA-9090, Debio 0932, KW-2478, XL888, CNF2024, TAS-116
TpoR agonists: Eltrombopag (sold under the tradenames Promacta® and Revolade® by GlaxoSmithKline);
Anti-mitotic agents: Docetaxel (sold under the tradename Taxotere® by Sanofi-Aventis); Adrenal steroid inhibitors: aminoglutethimide (sold under the tradename Cytadren®);
Anti-androgens: Nilutamide (sold under the tradenames Nilandron® and Anandron®), bicalutamide (sold under tradename Casodex®), flutamide (sold under the tradename Fulexin™);
Androgens: Fluoxymesterone (sold under the tradename Halotestin®);
CDK (CDK1, CDK2, CDK3, CDK5, CDK7, CDK8, CDK9, CDK11/12, or CDK16) inhibitors including but not limited to Alvocidib (pan-CDK inhibitor, also known as flovopirdol or HMR-1275, 2-(2-chlorophenyl)-5,7-dihydroxy-8-[(3S,4R)-3-hydroxy-1-methyl-4-piperidinyl]-4-chromenone, and described in U.S. Pat. No. 5,621,002);
CDK4/6 inhibitors pabociclib, ribociclib, abemaciclib, and Trilaciclib; CDK9 inhibitors AZD 4573, P276-00, AT7519M, TP-1287; CDK2/4/6 inhibitor such as PF-06873600;
SHP-2 inhibitor such as TNO155;
MDM2/MDMX, MDM2/p53 and/or MDMX/p53 modulators;
Gonadotropin-releasing hormone (GnRH) receptor agonists: Leuprolide or leuprolide acetate (sold under the tradenames Viadure® by Bayer AG, Eligard® by Sanofi-Aventis and Lupron® by Abbott Lab);
Taxane anti-neoplastic agents: Cabazitaxel (1-hydroxy-7,10-dimethoxy-9-oxo-5,20-epoxytax-11-ene-2a,4,13a-triyl-4-acetate-2-benzoate-13-[(2R,3S)-3-{[(tert-butoxy)carbonyl]amino}-2-hydroxy-3-phenylpropanoate), larotaxel ((2α,3ξ,4α,5β,7α,10β,13α)-4,10-bis(acetyloxy)-13-({(2R,3S)-3-[(tert-butoxycarbonyl) amino]-2-hydroxy-3-phenylpropanoyl)oxy)-1-hydroxy-9-oxo-5,20-epoxy-7,19-cyclotax-11-en-2-yl benzoate);
5HT1a receptor agonists: Xaliproden (also known as SR57746, 1-[2-(2-naphthyl)ethyl]-4-[3-(trifluoromethyl)phenyl]-1,2,3,6-tetrahydropyridine, and described in U.S. Pat. No. 5,266,573); HPC vaccines: Cervarix® sold by GlaxoSmithKline, Gardasil® sold by Merck; Iron Chelating agents: Deferasinox (sold under the tradename Exjade® by Novartis);
Anti-metabolites: Claribine (2-chlorodeoxyadenosine, sold under the tradename Leustatin®), 5-fluorouracil (sold under the tradename Adrucil®), 6-thioguanine (sold under the tradename Purinethol®), pemetrexed (sold under the tradename Alimta®), cytarabine (also known as arabinosylcytosine (Ara-C), sold under the tradename Cytosar-U®), cytarabine liposomal (also known as Liposomal Ara-C, sold under the tradename DepoCyt™), decitabine (sold under the tradename Dacogen®), hydroxyurea (sold under the tradenames Hydrea®, Droxia™ and Mylocel™), fludarabine (sold under the tradename Fludara®), floxuridine (sold under the tradename FUDR®), cladribine (also known as 2-chlorodeoxyadenosine (2-CdA) sold under the tradename Leustatin™), methotrexate (also known as amethopterin, methotrexate sodium (MTX), sold under the tradenames Rheumatrex® and Trexall™), pentostatin (sold under the tradename Nipent®);
Bisphosphonates: Pamidronate (sold under the tradename Aredia®), zoledronic acid (sold under the tradename Zometa®); Demethylating agents: 5-azacitidine (sold under the tradename Vidaza®), decitabine (sold under the tradename Dacogen®);
Plant Alkaloids: Paclitaxel protein-bound (sold under the tradename Abraxane®), vinblastine (also known as vinblastine sulfate, vincaleukoblastine and VLB, sold under the tradenames Alkaban-AQ® and Velban®), vincristine (also known as vincristine sulfate, LCR, and VCR, sold under the tradenames Oncovin® and Vincasar Pfs®), vinorelbine (sold under the tradename Navelbine®), paclitaxel (sold under the tradenames Taxol and Onxal™);
Retinoids: Ali tretinoin (sold under the tradename Panretin®), tretinoin (all-trans retinoic acid, also known as ATRA, sold under the tradename Vesanoid®), Isotretinoin (13-cis-retinoic acid, sold under the tradenames Accutane®, Amnesteem®, Claravis®, Clarus®, Decutan®, Isotane®, Izotech®, Oratane®, Isotret®, and Sotret®, bexarotene (sold under the tradename Targretin®);
Glucocorticosteroids: Hydrocortisone (also known as cortisone, hydrocortisone sodium succinate, hydrocortisone sodium phosphate, and sold under the tradenames Ala-Cort®, Hydrocortisone Phosphate, Solu-Cortef®, Hydrocort Acetate® and Lanacort), dexamethazone ((8S,9R,10S,11S,13S,14S,16R,17R)-9-fluoro-11,17-dihydroxy-17-(2-hydroxyacetyl)-10,13,16-trimethyl-6,7,8,9,10,11,12,13,14,15,16,17-dodecahydro-3H-cyclopenta[a]phenanthren-3-one), prednisolone (sold under the tradenames Delta-Cortel®, Orapred®, Pediapred® and Prelone®), prednisone (sold under the tradenames Deltasone®, Liquid Red®, Meticorten® and Orasone®), methylprednisolone (also known as 6-Methylprednisolone, Methylprednisolone Acetate, Methylprednisolone Sodium Succinate, sold under the tradenames Duralone®, Medralone®, Medrol®, M-Prednisol® and Solu-Medrol®);
Cytokines: interleukin-2 (also known as aldesleukin and IL-2, sold under the tradename Proleukin®), interleukin-11 (also known as oprevelkin, sold under the tradename Neunega®), alpha interferon alfa (also known as IFN-alpha, sold under the tradenames Intron® A, and Roferon-A®); [00209] Estrogen receptor downregulators: Fulvestrant (sold under the tradename Faslodex®);
Anti-estrogens: tamoxifen (sold under the tradename Novaldex®); Toremifene (sold under the tradename Fareston®);
Selective estrogen receptor modulators (SERMs): Raloxifene (sold under the tradename Evista®);
Leutinizing hormone releasing hormone (LHRH) agonists: Goserelin (sold under the tradename Zoladex®); Progesterones: megestrol (also known as megestrol acetate, sold under the tradename Megacef);
Miscellaneous cytotoxic agents: Arsenic trioxide (sold under the tradename Trisenox®), asparaginase (also known as L-asparaginase, Erwinia L-asparaginase, sold under the tradenames Elspar® and Kidrolase);

One or more immune checkpoint inhibitors CD27, CD28, CD40, CD122, CD96, CD73, CD39, CD47, OX40, GITR, CSF1R, JAK, PI3K delta, PI3K gamma, TAM kinase, arginase, CD137 (also known as 4-1BB), ICOS, A2AR, A2BR, HIF-2a, B7-H3, B7-H4, BTLA, CTLA-4, LAG3, TIM3, VISTA, CD96, TIGIT, PD-1, PD-L1 and PD-L2. In some embodiments, the immune checkpoint molecule is a stimulatory checkpoint molecule selected from CD27, CD28, CD40, ICOS, OX40, GITR, CD137 and STING. In some embodiments, the immune checkpoint molecule is an inhibitory checkpoint molecule selected from B7-H3, B7-H4, BTLA, CTLA-4, IDO, TDO, Arginase, KIR, LAG3, PD-1, TIM3, CD96, TIGIT and VISTA. In some embodiments, the compounds provided herein can be used in combination with one or more agents selected from KIR inhibitors, TIGIT inhibitors, LAIR1 inhibitors, CD160 inhibitors, 2B4 inhibitors and TGFR beta inhibitors.

In some embodiments, the inhibitor of an immune checkpoint molecule is an inhibitor of PD-1, e.g., an anti-PD-1 monoclonal antibody. In some embodiments, the anti-PD-1 monoclonal antibody is nivolumab, pembrolizumab (also known as MK-3475), pidilizumab, SHR-1210, PDR001, or AMP-224. In some embodiments, the anti-PD-1 monoclonal antibody is nivolumab, or pembrolizumab or PDR001. In some embodiments, the anti-PD1 antibody is pembrolizumab.

In some embodiments, the inhibitor of an immune checkpoint molecule is an inhibitor of PD-L1, e.g., an anti-PD-L1 monoclonal antibody. In some embodiments, the anti-PD-L1 monoclonal antibody is BMS-935559, MEDI4736, MPDL3280A (also known as RG7446), or MSB0010718C. In some embodiments, the anti-PD-L1 monoclonal antibody is MPDL3280A (atezolizumab) or MEDI4736 (durvalumab).

In some embodiments, the inhibitor of an immune checkpoint molecule is an inhibitor of CTLA-4, e.g., an anti-CTLA-4 antibody. In some embodiments, the anti-CTLA-4 antibody is ipilimumab or tremelimumab. In some embodiments, the inhibitor of an immune checkpoint molecule is an inhibitor of LAG3, e.g., an anti-LAG3 antibody. In some embodiments, the anti-LAG3 antibody is BMS-986016 or LAG525. In some embodiments, the inhibitor of an immune checkpoint molecule is an inhibitor of GITR, e.g., an anti-GITR antibody. In some embodiments, the anti-GITR antibody is TRX518 or, MK-4166, INCAGN01876 or MK-1248. In some embodiments, the inhibitor of an immune checkpoint molecule is an inhibitor of OX40, e.g., an anti-OX40 antibody or OX40L fusion protein. In some embodiments, the anti-OX40 antibody is MEDI0562 or, INCAGN01949, GSK2831781, GSK-3174998, MOXR-0916, PF-04518600 or LAG525. In some embodiments, the OX40L fusion protein is MEDI6383

Compounds of Formula (IA) or (I) can also be used to increase or enhance an immune response, including increasing the immune response to an antigen; to improve immunization, including increasing vaccine efficacy; and to increase inflammation. In some embodiments, the compounds of the invention can be sued to enhance the immune response to vaccines including, but not limited, Listeria vaccines, oncolytic viral vaccines, and cancer vaccines such as GVAX® (granulocyte-macrophage colony-stimulating factor (GM-CF) gene-transfected tumor cell vaccine). Anti-cancer vaccines include dendritic cells, synthetic peptides, DNA vaccines and recombinant viruses. Other immune-modulatory agents also include those that block immune cell migration such as antagonists to chemokine receptors, including CCR2 and CCR4; Sting agonists and Toll receptor agonists.

Other anti-cancer agents also include those that augment the immune system such as adjuvants or adoptive T cell transfer. Compounds of this application may be effective in combination with CAR (Chimeric antigen receptor) T cell treatment as a booster for T cell activation.

A compound of Formula (IA) or (I) can also be used in combination with the following adjunct therapies: anti-nausea drugs: NK-1 receptor antagonists: Casopitant (sold under the tradenames Rezonic® and Zunrisa® by GlaxoSmithKline); and Cytoprotective agents: Amifostine (sold under the tradename Ethyol®), leucovorin (also known as calcium leucovorin, citrovorum factor and folinic acid).

### EXAMPLES

The following preparations of compounds of Formula (IA′) are given to enable those skilled in the art to more clearly understand and to practice the present disclosure. They should not be considered as limiting the scope of the disclosure, but merely as being illustrative and representative thereof.

### Example 1

### Synthesis of 4-((8-cyclopentyl-7-oxo-7,8-dihydropyrido[2,3-d]pyrimidin-2-yl)amino)-N-(14-((2-(2,6-dioxopiperidin-3-yl)-1,3-dioxoisoindolin-4-yl)amino)-3,6,9,12-tetraoxatetradecyl)-piperidine-1-sulfonamide

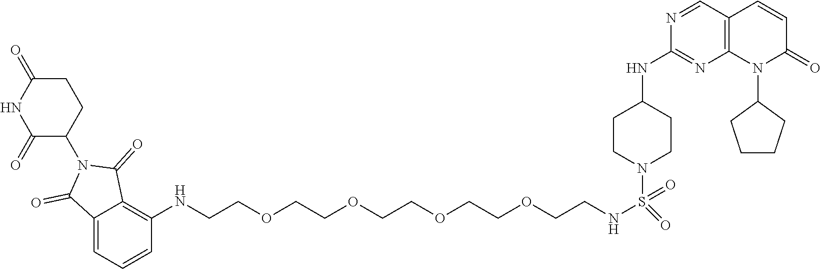 [SMILES: O=C1CCC(N2C(=O)c3cccc(NCCOCCOCCOCCOCCNS(=O)(=O)N4CCC(Nc5ncc6ccc(=O)n(C7CCCC7)c6n5)CC4)c3C2=O)C(=O)N1]

### Step 1: (4-cyclopentylamino-2-methylsulfanyl-pyrimidin-5-yl)-methanol

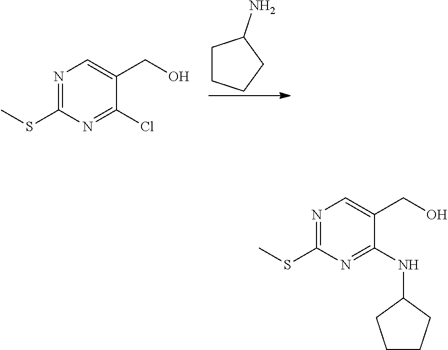

To a stirred solution of (4-chloro-2-methylsulfanyl-pyrimidin-5-yl)-methanol (570 mg, 2.99 mmol, 1.00 eq.) and cyclopentylamine (383 mg, 4.50 mmol, 1.51 eq.) in i-PrOH (15.0 mL) was added DIPEA (1.16 g, 8.97 mmol, 3.00 eq.). The resulting mixture was stirred at 80° C. for 16 h, concentrated and purified by silica gel column chromatography eluting with PE/EtOAc (1:1) to give title compound (500 mg, 69.9%) as yellow solid.

### Step 2: 4-cyclopentylamino-2-methylsulfanyl-pyrimidine-5-carbaldehyde

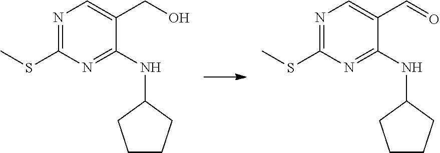 [SMILES: CSc1ncc(CO)c(NC2CCCC2)n1 ; CSc1ncc(C=O)c(NC2CCCC2)n1]

To a stirred solution of (4-cyclopentylamino-2-methylsulfanyl-pyrimidin-5-yl)-methanol (500 mg, 2.09 mmol, 1.00 eq.) in DCM (30.0 mL) was added MnO₂ (1.83 g, 21.05 mmol, 10.00 eq.). The resulting mixture was stirred at RT for 16 h, filtered and concentrated to give the title compound (450 mg, 90.9%) as yellow oil.

### Step 3: 8-cyclopentyl-2-methylsulfanyl-8H-pyrido[2,3-d]pyrimidin-7-one

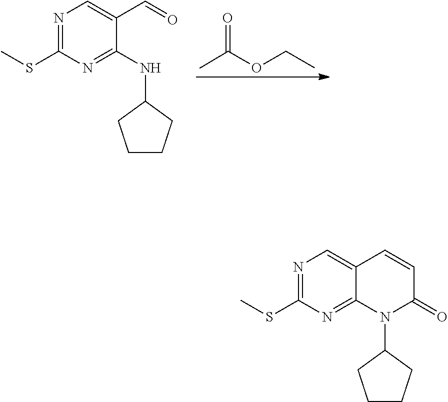

To a stirred solution of ethyl acetate (167 mg, 1.90 mmol, 1.00 eq.) and 4-cyclopentylamino-2-methylsulfanyl-pyrimidine-5-carbaldehyde (450 mg, 1.90 mmol, 1.00 eq.) in THF (5.0 mL) was added 1.0 M LiHMDS (5.70 mL, 5.70 mmol, 3.00 eq.) slowly at −78° C. The reaction mixture was warmed slowly to RT, stirred at RT for 16 h, quenched with H₂O and then extracted with EtOAc. The organic layer was concentrated and purified by silica gel column chromatography eluting with PE/EtOAc (12:1) to give the title compound (200 mg, 40.5%) as yellow oil.

### Step 4: 8-cyclopentyl-2-(methylsulfonyl)pyrido[2,3-d]pyrimidin-7 (8H)-one

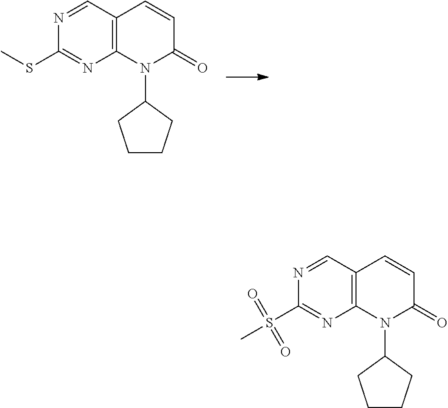 [SMILES: CSc1ncc2ccc(=O)n(C3CCCC3)c2n1 ; CS(=O)(=O)c1ncc2ccc(=O)n(C3CCCC3)c2n1]

To a stirred solution of 8-cyclopentyl-2-(methylthio)pyrido[2,3-d]pyrimidin-7 (8H)-one (3.00 g, 11.48 mmol, 1.00 eq.) in DCM (30.0 mL) was added m-CPBA (77%, 4.66 g, 20.79 mmol, 1.81 eq.) in portions at 5° C. The reaction mixture was stirred at RT for 16 h, diluted with water and extracted with DCM. The combined organic layer was washed with water, aqueous Na₂CO₃, water and brine. The organic layer was concentrated and the residue was purified by silica gel column chromatography, eluted with EtOAc/PE (1:10), to give the crude product, which was triturated with EtOAc/PE (1:10) to afford the title compound (2.2 g, 65.3%) as a white solid.

### Step 5: tert-butyl 4-((8-cyclopentyl-7-oxo-7,8-dihydropyrido[2,3-d]pyrimidin-2-yl)amino)-piperidine-1-carboxylate

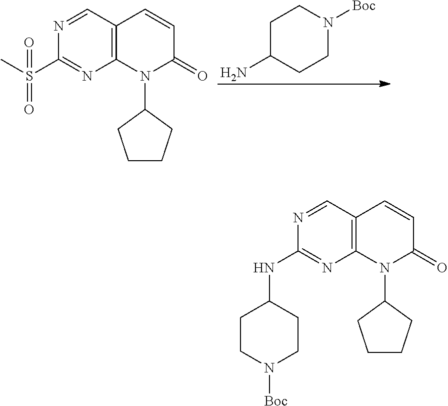

A mixture of 8-cyclopentyl-2-(methylsulfonyl)pyrido[2,3-d]pyrimidin-7 (8H)-one (750 mg, 2.56 mmol, 1.00 eq.), tert-butyl 4-aminopiperidine-1-carboxylate (512.1 mg, 2.56 mmol, 1.00 eq.) and DIPEA (991.3 mg, 7.67 mmol, 3.00 eq.) in i-PrOH (8.0 mL) was stirred for 2 h at 80° C. under nitrogen atmosphere. The resulting mixture was diluted with water and extracted with EtOAc. The combined organic layer was washed with brine, dried over anhydrous Na₂SO₄ and concentrated. The residue was purified by silica gel column chromatography, eluted with PE/EtOAc (5:1), to afford the title compound (900 mg, 85.1%) as a yellow solid.

### Step 6: 8-cyclopentyl-2-(piperidin-4-ylamino)pyrido[2,3-d]pyrimidin-7 (8H)-one

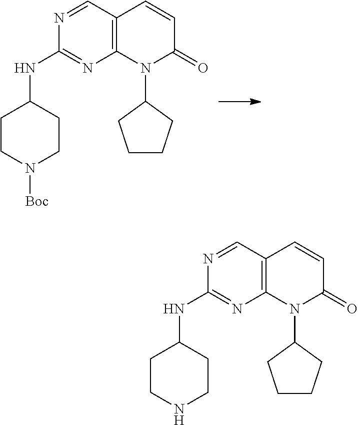 [SMILES: CC(C)(C)OC(=O)N1CCC(Nc2ncc3ccc(=O)n(C4CCCC4)c3n2)CC1 ; O=c1ccc2cnc(NC3CCNCC3)nc2n1C1CCCC1]

A solution of tert-butyl 4-((8-cyclopentyl-7-oxo-7,8-dihydropyrido[2,3-d]pyrimidin-2-yl)amino)piperidine-1-carboxylate (700 mg, 1.69 mmol, 1.00 eq.) and TFA (6.3 mL, 82.27 mmol, 48.68 eq.) in DCM (10 mL) was stirred for 3 h at room temperature under nitrogen atmosphere. The resulting mixture was concentrated, treated with water and basified to pH=9 with saturated Na₂CO₃ (aq.). The resulting mixture was extracted with DCM. The combined organic layer was washed with brine, dried over anhydrous Na₂SO₄ and concentrated to give the title compound (420 mg, 79.3%) as a light yellow solid.

### Step 7: 4-((8-cyclopentyl-7-oxo-7,8-dihydropyrido[2,3-d]pyrimidin-2-yl)amino)piperidine-1-sulfonyl chloride

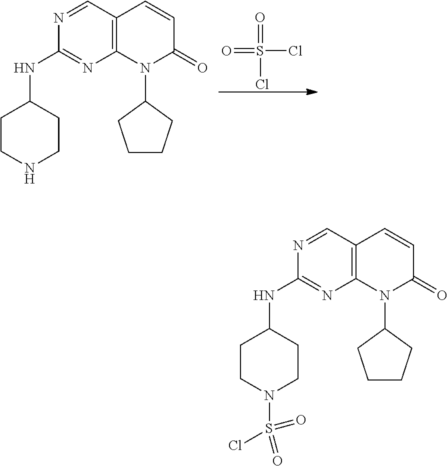

To a stirred solution of 8-cyclopentyl-2-(piperidin-4-ylamino)pyrido[2,3-d]pyrimidin-7 (8H)-one (125.0 mg, 0.40 mmol, 1.00 eq.) and DIPEA (206.2 mg, 1.60 mmol, 4.00 eq.) in DCM (2.0 mL) was added a solution of sulfonyl chloride (80.7 mg, 0.60 mmol, 1.50 eq.) in DCM (1.0 mL) dropwise at −70° C. under nitrogen atmosphere. The resulting mixture was stirred for 1 h at −30° C., quenched with water at 0° C., and then extracted with DCM. The combined organic layer was washed with brine, dried over anhydrous Na₂SO₄, filtered and then concentrated. The residue was purified by silica gel column chromatography, eluted with PE/EtOAc (1:1), to afford the title compound (80 mg, 47.5%) as a white solid.

### Step 8: 2-(2,6-dioxopiperidin-3-yl)-4-fluoroisoindoline-1,3-dione

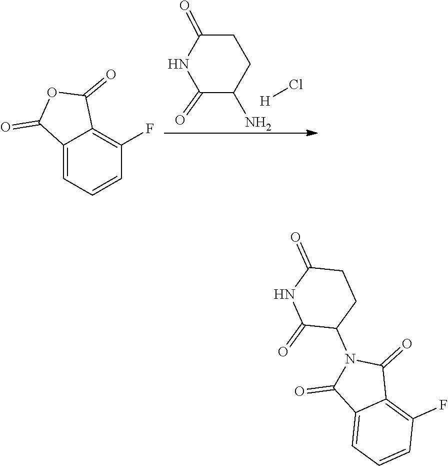

To a stirred mixture of 4-fluoroisobenzofuran-1,3-dione (3.32 g, 19.987 mmol, 1.00 equiv) and 3-aminopiperidine-2,6-dione hydrochloride (3.29 g, 19.99 mmol, 1.00 eq.) in AcOH (60.0 mL) was added NaOAc (1.97 g, 23.98 mmol, 1.20 eq.) at room temperature under nitrogen atmosphere. The resulting mixture was stirred for 16 h at 110° C., cooled, and then concentrated. The residue was triturated with water, filtered, and the solid cake was washed with water. The solid was dried to give the title compound (5.0 g, 90.5%) as a white solid

### Step 9: tert-butyl (14-((2-(2,6-dioxopiperidin-3-yl)-1,3-dioxoisoindolin-4-yl)amino)-3,6,9,12-tetraoxatetradecyl)carbamate

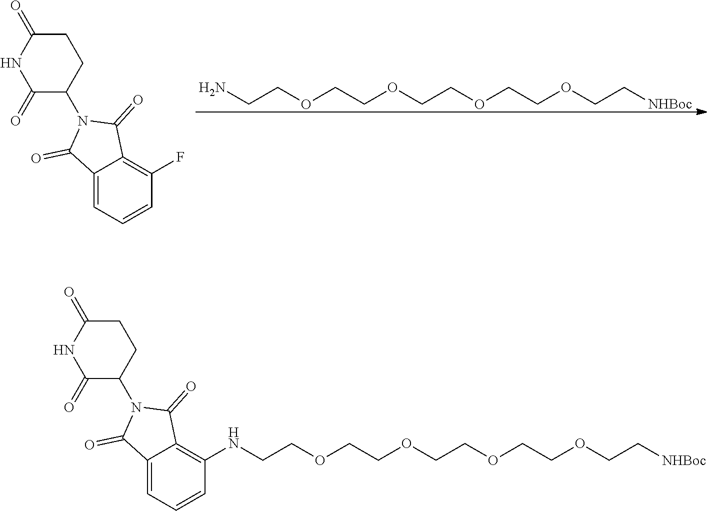 [SMILES: O=C1CCC(N2C(=O)c3cccc(F)c3C2=O)C(=O)N1 ; CC(C)(C)OC(=O)NCCOCCOCCOCCOCCN ; CC(C)(C)OC(=O)NCCOCCOCCOCCOCCNc1cccc2c1C(=O)N(C1CCC(=O)NC1=O)C2=O]

To a stirred mixture of 2-(2,6-dioxopiperidin-3-yl)4-fluoroisoindoline-1,3-dione (500.0 mg, 1.81 mmol, 1.00 eq.) and tert-butyl (14-amino-3,6,9,12-tetraoxatetradecyl)carbamate (609.0 mg, 1.81 mmol, 1.00 eq.) in DMF (6.0 mL) was added DIPEA (467.9 mg, 3.62 mmol, 2.00 eq.) at RT under nitrogen atmosphere. The resulting mixture was stirred for 16 h at 90° C. under nitrogen atmosphere, cooled, diluted with water, and then extracted with EtOAc. The combined organic layer was washed with water, dried over anhydrous Na₂SO₄, filtered, and then concentrated. The residue was purified by silica gel column chromatography, eluted with PE/EtOAc (5:1), to afford the title compound (420 mg, 39.2%) as a yellow solid. MS (ES, m/z): [M+1]⁺=593.2.

### Step 10: 4-((14-amino-3,6,9,12-tetraoxatetradecyl)amino)-2-(2,6-dioxopiperidin-3-yl)isoindoline-1,3-dione, 2,2,2-trifluoroacetate

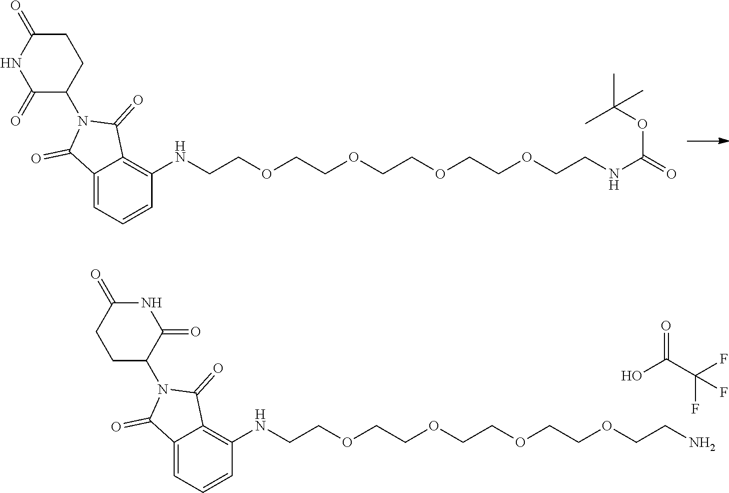

To a stirred solution of tert-butyl (14-((2-(2,6-dioxopiperidin-3-yl)-1,3-dioxoisoindolin-4-yl)amino)-3,6,9,12-tetraoxatetradecyl)carbamate (50 mg, 0.084 mmol, 1.00 eq.) in DCM (1.0 mL) was added TFA (0.3 mL, 3.92 mmol, 46.67 eq.) dropwise at 0° C. under nitrogen atmosphere. The resulting mixture was stirred for 2 h, and then concentrated to give crude title compound (50 mg, 97.6%) as light yellow oil.

### Step 11: 4-((8-cyclopentyl-7-oxo-7,8-dihydropyrido[2,3-d]pyrimidin-2-yl)amino)-N-(14-((2-(2,6-dioxopiperidin-3-yl)-1,3-dioxoisoindolin-4-yl)amino)-3,6,9,12-tetraoxatetradecyl)piperidine-1-sulfonamide

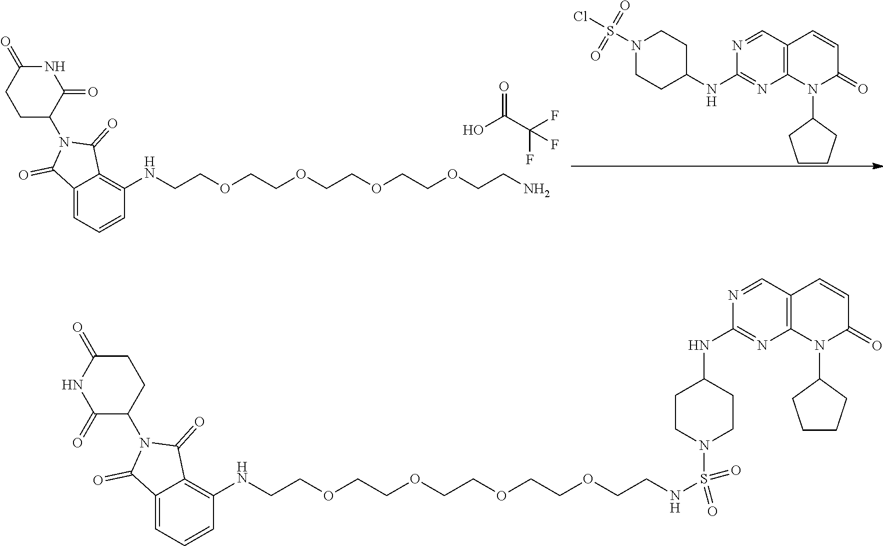

To a stirred solution of 4-((14-amino-3,6,9,12-tetraoxatetradecyl)amino)-2-(2,6-dioxopiperidin-3-yl)isoindoline-1,3-dione, 2,2,2-trifluoroacetate (35.9 mg, 0.059 mmol, 1.00 eq.), DMAP (8.9 mg, 0.073 mmol, 1.24 eq.) and DIPEA (28.2 mg, 0.22 mmol, 3.73 eq.) in DCM (1.0 mL) was added a solution of 4-((8-cyclopentyl-7-oxo-7,8-dihydropyrido[2,3-d]pyrimidin-2-yl)amino)piperidine-1-sulfonyl chloride (30.0 mg, 0.073 mmol, 1.24 eq.) in DCM (0.3 mL) dropwise at 0° C. under nitrogen atmosphere. The resulting mixture was stirred for 4 h at room temperature, and then concentrated. The crude product was purified by Prep-HPLC to afford the title compound (9 mg, 16.9%) as a yellow solid. MS (ES, m/z): [M+1]⁺=868.4.

### Example 2

### Synthesis of N-(2-(2-(2-(4-((4-((8-cyclopentyl-7-oxo-7,8-dihydropyrido[2,3-d]pyrimidin-2-yl)amino) piperidin-1-yl)sulfonyl)piperazin-1-yl)ethoxy)ethoxy)ethyl)-2-((2-(2,6-dioxopiperidin-3-yl)-1,3-dioxoisoindolin-4-yl)oxy)acetamide

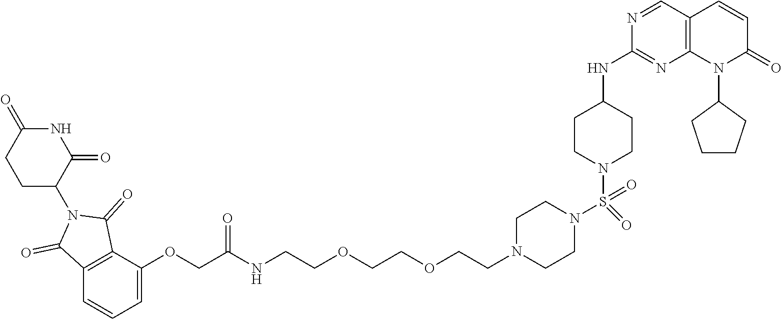 [SMILES: O=C(COc1cccc2c1C(=O)N(C1CCC(=O)NC1=O)C2=O)NCCOCCOCCN1CCN(S(=O)(=O)N2CCC(Nc3ncc4ccc(=O)n(C5CCCC5)c4n3)CC2)CC1]

### Step 1: tert-butyl 2-((2-(2,6-dioxopiperidin-3-yl)-1,3-dioxoisoindolin-4-yl)oxy)acetate

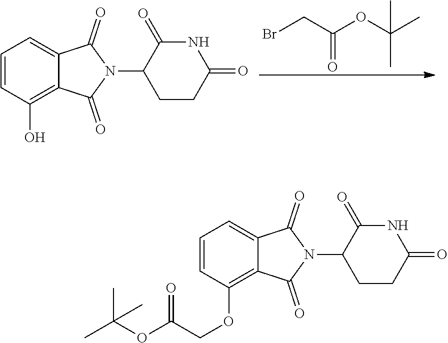

A mixture of 2-(2,6-dioxopiperidin-3-yl)-4-hydroxyisoindoline-1,3-dione (1.5 g, 5.47 mmol, 1.00 eq.), tert-butyl 2-bromoacetate (1.3 g, 6.66 mmol, 1.22 eq.) and K₂CO₃ (1.1 g, 7.96 mmol, 1.46 eq.) in DMF (20.0 mL) was stirred at RT for 2 h. The reaction mixture was diluted with H₂O and extracted with ethyl acetate. The combined organic phase was washed with brine, dried over Na₂SO₄, concentrated to get title compound (1.2 g, 56.5%) as a white solid.

### Step 2: 2-((2-(2,6-dioxopiperidin-3-yl)-1,3-dioxoisoindolin-4-yl)oxy)acetic acid

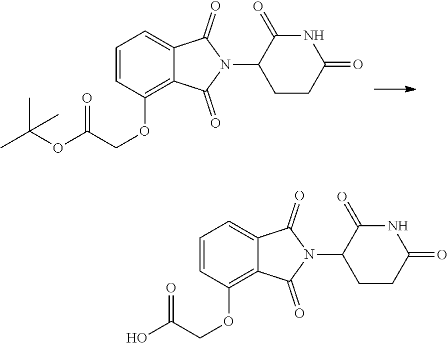 [SMILES: CC(C)(C)OC(=O)COc1cccc2c1C(=O)N(C1CCC(=O)NC1=O)C2=O ; O=C(O)COc1cccc2c1C(=O)N(C1CCC(=O)NC1=O)C2=O]

A solution of tert-butyl 2-((2-(2,6-dioxopiperidin-3-yl)-1,3-dioxoisoindolin-4-yl)oxy)-acetate (1.0 g, 2.57 mmol, 1.00 eq.) and TFA (5.0 mL) in DCM (10.0 mL) was stirred at RT for 2 h. The reaction mixture was concentrated and the residue was triturated with ether to get title compound (800 mg, 93.8%) as a white solid.

### Step 3: 2-((2-(2,6-dioxopiperidin-3-yl)-1,3-dioxoisoindolin-4-yl)oxy)-N-(2-(2-(2-hydroxyethoxy) ethoxy)ethyl)acetamide

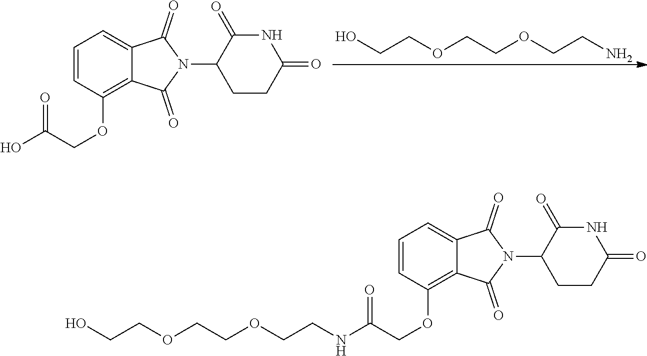

To a stirred solution of 2-((2-(2,6-dioxopiperidin-3-yl)-1,3-dioxoisoindolin-4-yl)oxy)acetic acid (300 mg, 0.90 mmol, 1.00 eq.), 2-(2-(2-aminoethoxy)ethoxy)ethanol (201 mg, 1.35 mmol, 1.50 eq.) and DIPEA (348 mg, 2.69 mmol, 2.99 eq.) in DMF (6.0 mL) was added HATU (513 mg, 1.35 mmol, 1.5 eq) at 0° C. The reaction mixture was stirred at RT for 1 h, diluted with H₂O and extracted with DCM. The combined organic phase was washed with brine, dried over Na₂SO₄, concentrated to get crude title compound (800 mg) as a yellow oil, which was used for next step without further purification.

### Step 4: 2-(2-(2-(2-((2-(2,6-dioxopiperidin-3-yl)-1,3-dioxoisoindolin-4-yl)oxy)acetamido)ethoxy) ethoxy)ethyl methanesulfonate

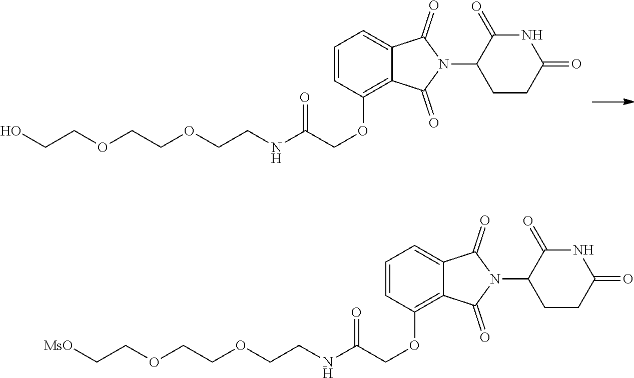 [SMILES: O=C(COc1cccc2c1C(=O)N(C1CCC(=O)NC1=O)C2=O)NCCOCCOCCO ; CS(=O)(=O)OCCOCCOCCNC(=O)COc1cccc2c1C(=O)N(C1CCC(=O)NC1=O)C2=O]

To a stirred solution of 2-((2-(2,6-dioxopiperidin-3-yl)-1,3-dioxoisoindolin-4-yl)oxy)-N-(2-(2-(2-hydroxyethoxy)ethoxy)ethyl)acetamide (800 mg, 1.73 mmol, 1.00 eq.) and TEA (524 mg, 5.18 mmol, 2.99 eq.) in DCM (8.0 mL) was added MsCl (298 mg, 2.60 mmol, 1.50 eq.) at 0° C. The reaction mixture was stirred at 0° C. for 1 h, diluted with H₂O and extracted with DCM. The combined organic phase was washed with brine, dried over Na₂SO₄, concentrated and purified by flash silica gel chromatography (DCM:MeOH=50:1) to get title compound (180 mg, 36.7% over two steps) as a white solid.

### Step 5: N-(2-(2-(2-(4-((4-((8-cyclopentyl-7-oxo-7,8-dihydropyrido[2,3-d]pyrimidin-2-yl)amino) piperidin-1-yl)sulfonyl)piperazin-1-yl)ethoxy)ethoxy)ethyl)-2-((2-(2,6-dioxopiperidin-3-yl)-1,3-dioxoisoindolin-4-yl)oxy)acetamide

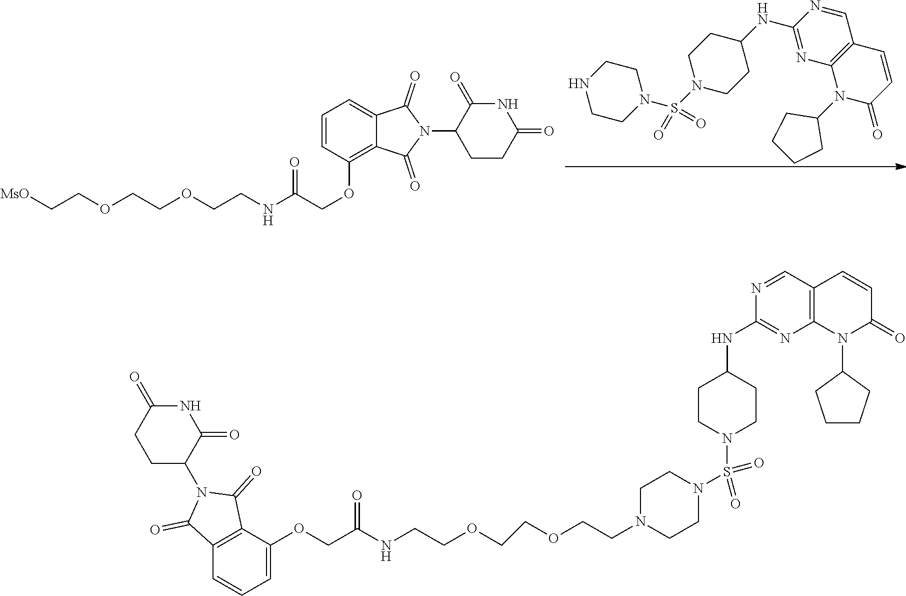 [SMILES: CS(=O)(=O)OCCOCCOCCNC(=O)COc1cccc2c1C(=O)N(C1CCC(=O)NC1=O)C2=O ; O=c1ccc2cnc(NC3CCN(S(=O)(=O)N4CCNCC4)CC3)nc2n1C1CCCC1 ; O=C(COc1cccc2c1C(=O)N(C1CCC(=O)NC1=O)C2=O)NCCOCCOCCN1CCN(S(=O)(=O)N2CCC(Nc3ncc4ccc(=O)n(C5CCCC5)c4n3)CC2)CC1]

A mixture of 2-(2-(2-(2-((2-(2,6-dioxopiperidin-3-yl)-1,3-dioxoisoindolin-4-yl)oxy)acetamido) ethoxy)ethoxy)ethyl methanesulfonate (70 mg, 0.13 mmol, 1.00 eq.), 8-cyclopentyl-2-((1-(piperazin-1-ylsulfonyl)piperidin-4-yl)amino)pyrido[2,3-d]pyrimidin-7 (8H)-one (60 mg, 0.13 mmol, 1.00 eq.), NaI (33 mg, 0.22 mmol, 1.69 eq.) and DIPEA (33 mg, 0.26 mmol, 2.00 eq.) in ACN (5.0 mL) was stirred at 80° C. overnight. The reaction mixture was concentrated and purified by Prep-HPLC to get title compound (28 mg, 23.8%) as a light-yellow solid. MS (ES, m/z): [M+1]⁺=907.4.

### Example 3

### Synthesis of N-(2-(2-(4-((4-((8-cyclopentyl-7-oxo-7,8-dihydropyrido[2,3-d]pyrimidin-2-yl)-amino)piperidin-1-yl)sulfonyl)piperazin-1-yl)ethoxy)ethyl)-2-((2-(2,6-dioxopiperidin-3-yl)-1,3-dioxoisoindolin-4-yl)oxy)acetamide

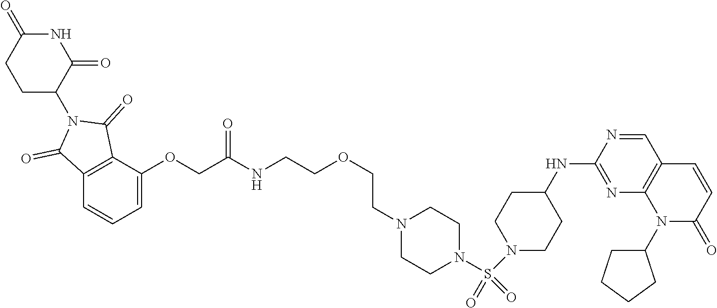 [SMILES: O=C(COc1cccc2c1C(=O)N(C1CCC(=O)NC1=O)C2=O)NCCOCCN1CCN(S(=O)(=O)N2CCC(Nc3ncc4ccc(=O)n(C5CCCC5)c4n3)CC2)CC1]

### Step 1: 2-((2-(2,6-dioxopiperidin-3-yl)-1,3-dioxoisoindolin-4-yl)oxy)-N-(2-(2-hydroxyethoxy)-ethyl) acetamide

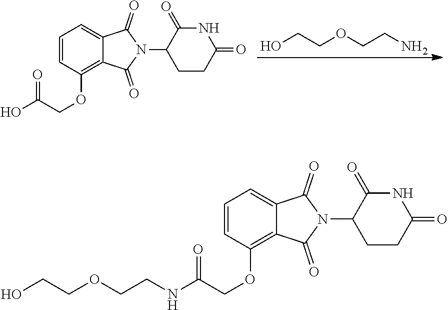

A mixture of 2-((2-(2,6-dioxopiperidin-3-yl)-1,3-dioxoisoindolin-4-yl)oxy)acetic acid (180 mg, 0.54 mmol, 1.00 eq.), 2-(2-aminoethoxy)ethan-1-ol (85 mg, 0.81 mmol, 1.50 eq.), HATU (308 mg, 0.81 mmol, 1.50 eq.) and DIPEA (209 mg, 1.62 mmol, 3.00 eq.) in DMF (5.0 mL) was stirred at 0° C. for 1 h. The reaction mixture was diluted with H₂O and extracted with DCM. The combined organic phase was washed with brine, dried over Na₂SO₄ and concentrated to get crude title compound (400 mg) as a yellow oil, which was used for next step without further purification.

### Step 2: 2-(2-(2-((2-(2,6-dioxopiperidin-3-yl)-1,3-dioxoisoindolin-4-yl)oxy)acetamido)ethoxy)-ethyl methanesulfonate

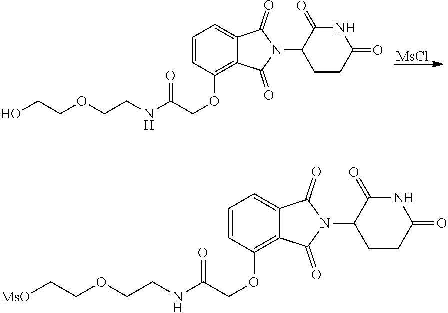 [SMILES: "MsCl" ; O=C(COc1cccc2c1C(=O)N(C1CCC(=O)NC1=O)C2=O)NCCOCCO ; CS(=O)(=O)OCCOCCNC(=O)COc1cccc2c1C(=O)N(C1CCC(=O)NC1=O)C2=O]

To a stirred solution of 2-((2-(2,6-dioxopiperidin-3-yl)-1,3-dioxoisoindolin-4-yl)oxy)-N-(2-(2-hydroxyethoxy)ethyl) acetamide (400 mg, 0.95 mmol, 1.00 eq.) and TEA (288 mg, 2.85 mmol, 3.00 eq.) in DCM (8.0 mL) was added MsCl (162 mg, 1.41 mmol, 1.48 eq.) slowly at 0° C. The resulting mixture was stirred at 0° C. for 1 h, diluted with H₂O and extracted with DCM. The combined organic phase was washed with brine, dried over Na₂SO₄, concentrated and purified by flash silica gel chromatography (DCM:MeOH=50:1) to give the title compound (80 mg, 29.6% over 2 steps) as a white solid.

### Step 3: N-(2-(2-(4-((4-((8-cyclopentyl-7-oxo-7,8-dihydropyrido[2,3-d]pyrimidin-2-yl)amino)-piperidin-1-yl)sulfonyl)piperazin-1-yl)ethoxy)ethyl)-2-((2-(2,6-dioxopiperidin-3-yl)-1,3-dioxoisoindolin-4-yl)oxy)acetamide

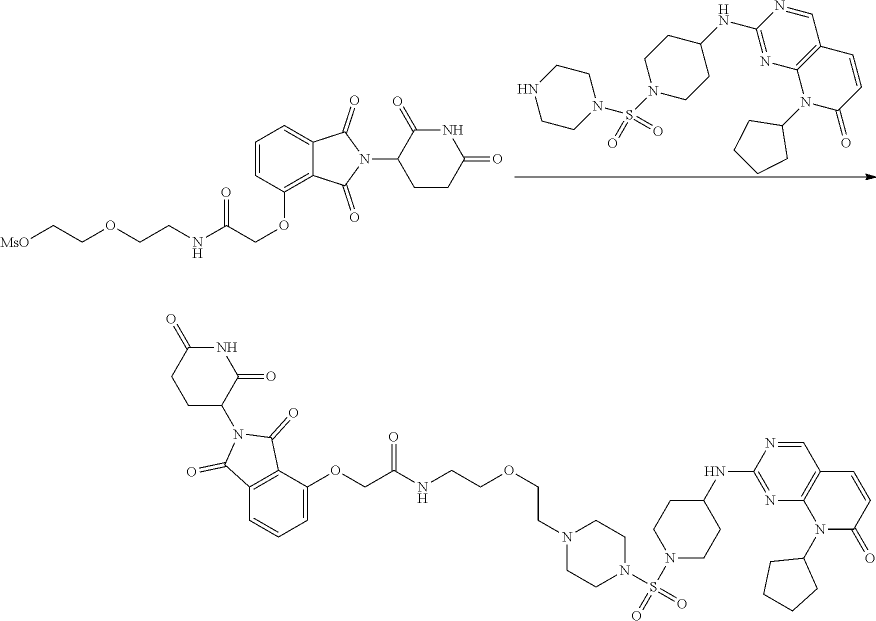 [SMILES: CS(=O)(=O)OCCOCCNC(=O)COc1cccc2c1C(=O)N(C1CCC(=O)NC1=O)C2=O ; O=c1ccc2cnc(NC3CCN(S(=O)(=O)N4CCNCC4)CC3)nc2n1C1CCCC1 ; O=C(COc1cccc2c1C(=O)N(C1CCC(=O)NC1=O)C2=O)NCCOCCN1CCN(S(=O)(=O)N2CCC(Nc3ncc4ccc(=O)n(C5CCCC5)c4n3)CC2)CC1]

A mixture of 2-((2-(2,6-dioxopiperidin-3-yl)-1,3-dioxoisoindolin-4-yl)oxy)-N-(2-(2-hydroxyethoxy)ethyl)acetamide (20 mg, 0.040 mmol, 1.03 eq.), 8-cyclopentyl-2-((1-(piperazin-1-ylsulfonyl)piperidin-4-yl)amino)pyrido[2,3-d]pyrimidin-7 (8H)-one (18 mg, 0.039 mmol, 1.00 eq.), NaI (6 mg, 0.040 mmol, 1.03 eq.) and DIPEA (15 mg, 0.12 mmol, 3.08 eq.) in ACN (3.0 mL) was stirred at 80° C. overnight. The mixture was cooled, concentrated and the residue was purified by flash silica gel chromatography (DCM:MeOH=30:1) to get the title compound (15 mg, 43.6%) as a yellow solid. MS (ES, m/z): [M+1]⁺=863.3.

### Example 4

### Synthesis of N-(2-(2-(2-(2-(4-((4-((8-cyclopentyl-7-oxo-7,8-dihydropyrido[2,3-d]pyrimidin-2-yl) amino)piperidin-1-yl)sulfonyl)piperazin-1-yl)ethoxy)ethoxy)ethoxy)ethyl)-2-((2-(2,6-dioxopiperidin-3-yl)-1,3-dioxoisoindolin-4-yl)oxy)acetamide

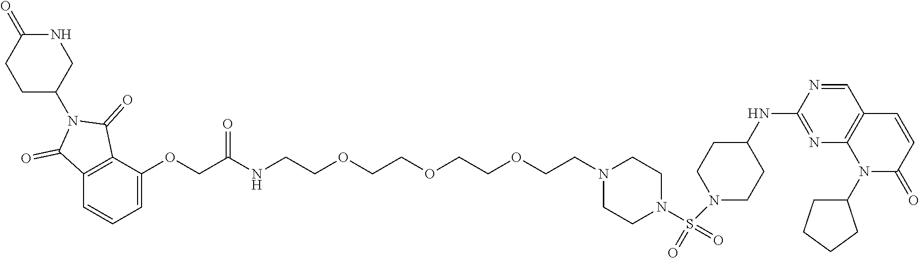 [SMILES: O=C1CCC(N2C(=O)c3cccc(OCC(=O)NCCOCCOCCOCCN4CCN(S(=O)(=O)N5CCC(Nc6ncc7ccc(=O)n(C8CCCC8)c7n6)CC5)CC4)c3C2=O)CN1]

### Step 1: 2-((2-(2,6-dioxopiperidin-3-yl)-1,3-dioxoisoindolin-4-yl)oxy)-N-(2-(2-(2-(2-hydroxyethoxy) ethoxy)ethoxy)ethyl)acetamide

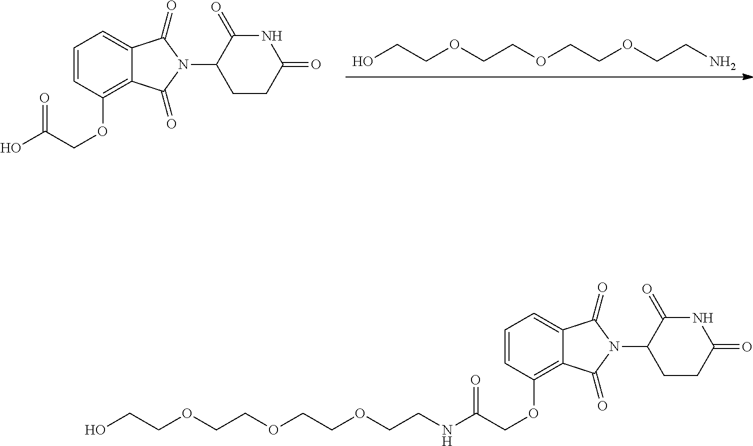 [SMILES: O=C(O)COc1cccc2c1C(=O)N(C1CCC(=O)NC1=O)C2=O ; NCCOCCOCCOCCO ; O=C(COc1cccc2c1C(=O)N(C1CCC(=O)NC1=O)C2=O)NCCOCCOCCOCCO]

A mixture of 2-((2-(2,6-dioxopiperidin-3-yl)-1,3-dioxoisoindolin-4-yl)oxy)acetic acid (300 mg, 0.90 mmol, 1.00 eq.) and 2-(2-(2-(2-aminoethoxy)ethoxy)ethoxy)ethan-1-ol (259 mg, 1.34 mmol, 1.49 eq.) in DMF (5.0 mL) were added HATU (513 mg, 1.35 mmol, 1.50 eq.) and DIPEA (348 mg, 2.69 mmol, 3.00 eq.) at 0° C. The resulting mixture was stirred at 0° C. for 1 h, diluted with H₂O, and then extracted with DCM. The combined organic phase was washed with brine, dried over Na₂SO₄, filtered and concentrated to get crude title compound (800 mg) as a yellow oil, which was used for next step without further purification.

### Step 2: 1-((2-(2,6-dioxopiperidin-3-yl)-1,3-dioxoisoindolin-4-yl)oxy)-2-oxo-6,9,12-trioxa-3-azatetradecan-14-yl methanesulfonate

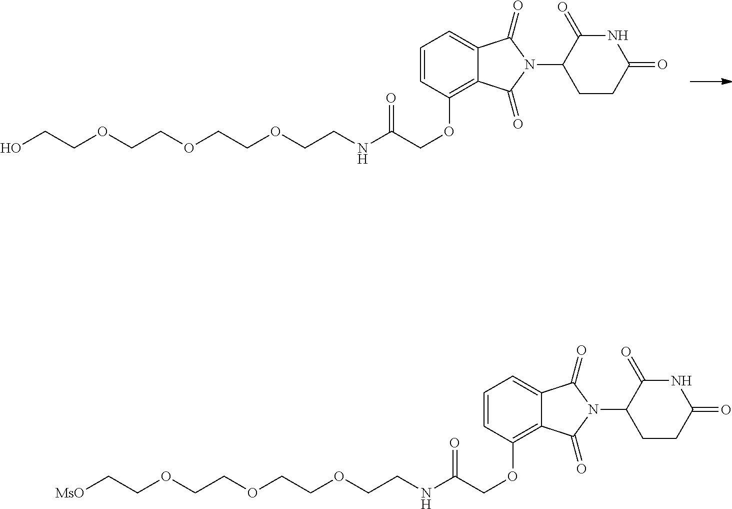 [SMILES: O=C(COc1cccc2c1C(=O)N(C1CCC(=O)NC1=O)C2=O)NCCOCCOCCOCCO ; CS(=O)(=O)OCCOCCOCCOCCNC(=O)COc1cccc2c1C(=O)N(C1CCC(=O)NC1=O)C2=O]

To a stirred solution of 2-((2-(2,6-dioxopiperidin-3-yl)-1,3-dioxoisoindolin-4-yl)oxy)-N-(2-(2-(2-(2-hydroxyethoxy) ethoxy)ethoxy)ethyl)acetamide (800 mg, 1.58 mmol, 1.00 eq.) and TEA (479 mg, 4.73 mmol, 3.00 eq.) in DCM (8.0 mL) was added MsCl (271 mg, 2.37 mmol, 1.50 eq.) slowly at 0° C. The resulting mixture was stirred at 0° C. for 1 h, diluted with H₂O and extracted with DCM. The combined organic phase was washed with brine, dried over Na₂SO₄, concentrated and purified by flash silica gel chromatography (DCM:MeOH=50:1) to get the title compound (180 mg, 19.6%) as a white solid.

### Step 3: N-(2-(2-(2-(2-(4-((4-((8-cyclopentyl-7-oxo-7,8-dihydropyrido[2,3-d]pyrimidin-2-yl) amino)piperidin-1-yl)sulfonyl)piperazin-1-yl)ethoxy)ethoxy)ethoxy)ethyl)-2-((2-(2,6-dioxopiperidin-3-yl)-1,3-dioxoisoindolin-4-yl)oxy)acetamide

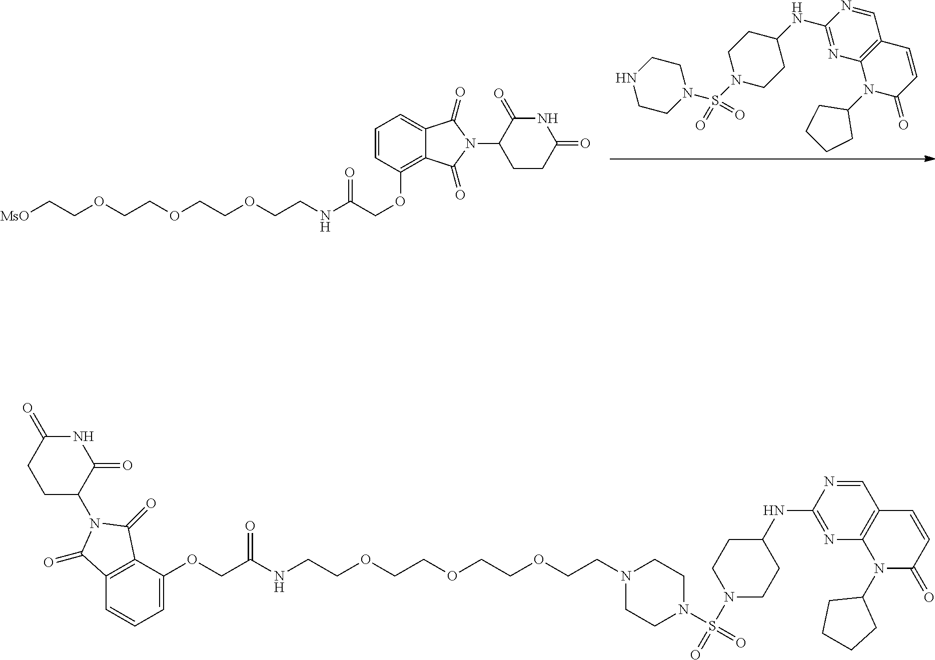 [SMILES: CS(=O)(=O)OCCOCCOCCOCCNC(=O)COc1cccc2c1C(=O)N(C1CCC(=O)NC1=O)C2=O ; O=c1ccc2cnc(NC3CCN(S(=O)(=O)N4CCNCC4)CC3)nc2n1C1CCCC1 ; O=C(COc1cccc2c1C(=O)N(C1CCC(=O)NC1=O)C2=O)NCCOCCOCCOCCN1CCN(S(=O)(=O)N2CCC(Nc3ncc4ccc(=O)n(C5CCCC5)c4n3)CC2)CC1]

A mixture of 1-((2-(2,6-dioxopiperidin-3-yl)-1,3-dioxoisoindolin-4-yl)oxy)-2-oxo-6,9,12-trioxa-3-azatetradecan-14-yl methanesulfonate (76 mg, 0.13 mmol, 1.00 eq.), 8-cyclopentyl-2-((1-(piperazin-1-ylsulfonyl)piperidin-4-yl)amino)pyrido[2,3-d]pyrimidin-7 (8H)-one (60 mg, 0.13 mmol, 1.00 eq.), NaI (20 mg, 0.13 mmol, 1.00 eq.) and DIPEA (33 mg, 0.26 mmol, 2.00 eq.) in ACN (5.0 mL) was stirred at 80° C. overnight. The reaction mixture was concentrated and the residue was purified by flash silica gel chromatography (DCM:MeOH=30:1) to get the title compound (26 mg, 20.8%) as a yellow solid.

### Example 5

### Synthesis of N-(2-(2-(4-((4-((8-cyclopentyl-7-oxo-7,8-dihydropyrido[2,3-d]pyrimidin-2-yl)amino) piperidin-1-yl)sulfonyl)piperazin-1-yl)ethoxy)ethyl)-2-((2-(1-methyl-2,6-dioxopiperidin-3-yl)-1,3-dioxoisoindolin-4-yl)oxy)acetamide

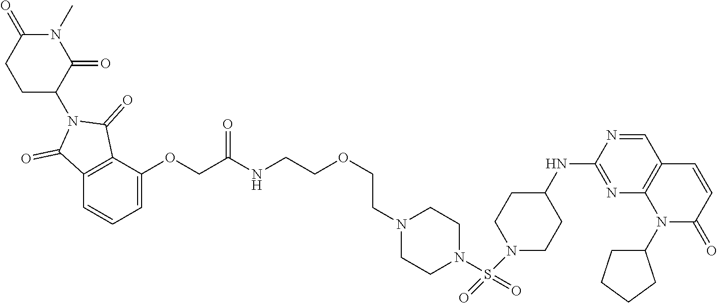 [SMILES: CN1C(=O)CCC(N2C(=O)c3cccc(OCC(=O)NCCOCCN4CCN(S(=O)(=O)N5CCC(Nc6ncc7ccc(=O)n(C8CCCC8)c7n6)CC5)CC4)c3C2=O)C1=O]

### Step 1: tert-butyl 2-((2-(1-methyl-2,6-dioxopiperidin-3-yl)-1,3-dioxoisoindolin-4-yl)oxy)acetate

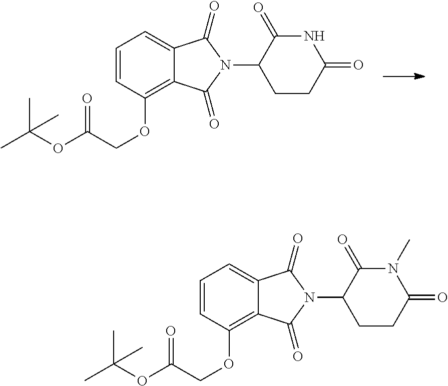 [SMILES: CC(C)(C)OC(=O)COc1cccc2c1C(=O)N(C1CCC(=O)NC1=O)C2=O ; CN1C(=O)CCC(N2C(=O)c3cccc(OCC(=O)OC(C)(C)C)c3C2=O)C1=O]

To a stirred solution of tert-butyl 2-((2-(2,6-dioxopiperidin-3-yl)-1,3-dioxoisoindolin-4-yl)oxy) acetate (500 mg, 1.29 mmol, 1.00 eq.), MeOH (125 mg, 3.90 mmol, 3.02 eq.) and PPh₃ (681 mg, 2.60 mmol, 2.02 eq.) in THF (80.0 mL) was added di-tert-butyl azodicarboxylate (DBAD, 897 mg, 3.90 mmol, 3.02 eq.) slowly at 0° C. The resulting mixture was stirred at RT overnight, diluted with H₂O and extracted with ethyl acetate. The combined organic phase was washed with brine, dried over Na₂SO₄, concentrated and purified by flash silica gel chromatography (DCM:MeOH=100:1) to get the title compound (400 mg, 76.7%) as a yellow oil.

### Step 2: 2-((2-(1-methyl-2,6-dioxopiperidin-3-yl)-1,3-dioxoisoindolin-4-yl)oxy)acetic acid

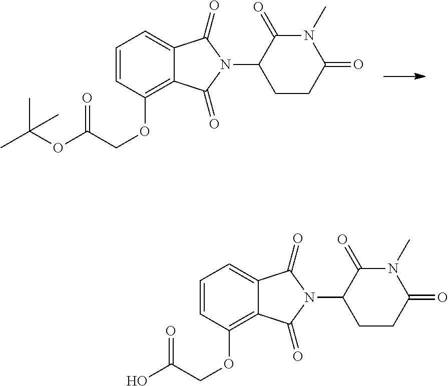 [SMILES: CN1C(=O)CCC(N2C(=O)c3cccc(OCC(=O)OC(C)(C)C)c3C2=O)C1=O ; CN1C(=O)CCC(N2C(=O)c3cccc(OCC(=O)O)c3C2=O)C1=O]

A solution of tert-butyl 2-((2-(1-methyl-2,6-dioxopiperidin-3-yl)-1,3-dioxoisoindolin-4-yl)oxy) acetate (400 mg, 0.99 mmol, 1.00 eq.) and TFA (2.0 mL) in DCM (4.0 mL) was stirred at RT for 1 h. The reaction mixture was concentrated and the residue was triturated with ether to get the title compound (350 mg, 100%) as a yellow solid.

### Step 3: N-(2-(2-hydroxyethoxy)ethyl)-2-((2-(1-methyl-2,6-dioxopiperidin-3-yl)-1,3-dioxoisoindolin-4-yl)oxy)acetamide

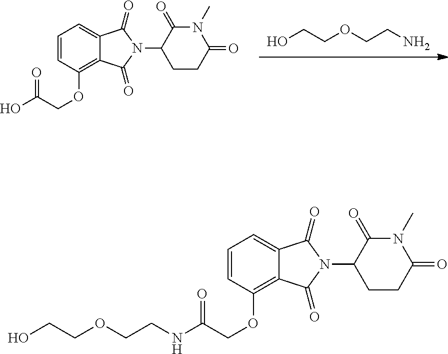 [SMILES: CN1C(=O)CCC(N2C(=O)c3cccc(OCC(=O)O)c3C2=O)C1=O ; NCCOCCO ; CN1C(=O)CCC(N2C(=O)c3cccc(OCC(=O)NCCOCCO)c3C2=O)C1=O]

A solution of 2-((2-(1-methyl-2,6-dioxopiperidin-3-yl)-1,3-dioxoisoindolin-4-yl)oxy)acetic acid (350 mg, 1.01 mmol, 1.00 eq.), 2-(2-aminoethoxy)ethan-1-ol (158 mg, 1.50 mmol, 1.49 eq.), DIPEA (387 mg, 2.99 mmol, 2.96 eq.) and HATU (570 mg, 1.50 mmol, 1.49 eq.) in DMF (6.0 mL) was stirred at 0° C. for 1 h. The reaction mixture was diluted with H₂O and extracted with DCM. The combined organic phase was washed with brine, dried over Na₂SO₄, concentrated to get crude title compound (700 mg) as a brown oil, which was used for next step without further purification.

### Step 4: 2-(2-(2-((2-(1-methyl-2,6-dioxopiperidin-3-yl)-1,3-dioxoisoindolin-4-yl)oxy)acetamido) ethoxy)ethyl methanesulfonate

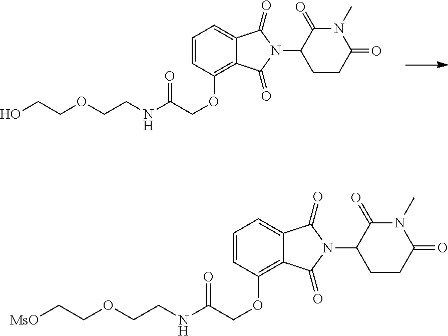 [SMILES: CN1C(=O)CCC(N2C(=O)c3cccc(OCC(=O)NCCOCCO)c3C2=O)C1=O ; CN1C(=O)CCC(N2C(=O)c3cccc(OCC(=O)NCCOCCOS(C)(=O)=O)c3C2=O)C1=O]

To a stirred solution of N-(2-(2-hydroxyethoxy)ethyl)-2-((2-(1-methyl-2,6-dioxopiperidin-3-yl)-1,3-dioxoisoindolin-4-yl)oxy)acetamide (700 mg, 1.62 mmol, 1.00 eq.) and TEA (485 mg, 4.79 mmol, 2.96 eq.) in DCM (8.0 mL) was added MsCl (275 mg, 2.40 mmol, 1.48 eq.) at 0° C. After stirring at 0° C. for 1 h, the reaction mixture was diluted with H₂O and extracted with DCM. The combined organic phase was washed with brine, dried over Na₂SO₄, concentrated and purified by flash silica gel chromatography (DCM:MeOH=30:1) to get the title compound (30 mg, 5.8% over 2 steps) as a white solid.

### Step 5: N-(2-(2-(4-((4-((8-cyclopentyl-7-oxo-7,8-dihydropyrido[2,3-d]pyrimidin-2-yl)amino) piperidin-1-yl)sulfonyl)piperazin-1-yl)ethoxy)ethyl)-2-((2-(1-methyl-2,6-dioxopiperidin-3-yl)-1,3-dioxoisoindolin-4-yl)oxy)acetamide

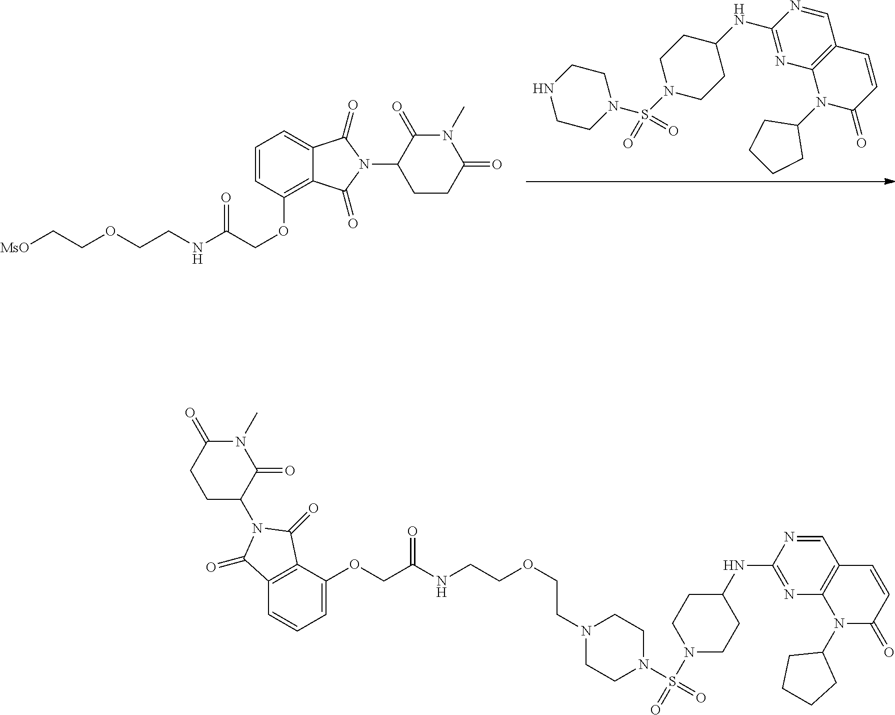 [SMILES: CN1C(=O)CCC(N2C(=O)c3cccc(OCC(=O)NCCOCCOS(C)(=O)=O)c3C2=O)C1=O ; O=c1ccc2cnc(NC3CCN(S(=O)(=O)N4CCNCC4)CC3)nc2n1C1CCCC1 ; CN1C(=O)CCC(N2C(=O)c3cccc(OCC(=O)NCCOCCN4CCN(S(=O)(=O)N5CCC(Nc6ncc7ccc(=O)n(C8CCCC8)c7n6)CC5)CC4)c3C2=O)C1=O]

A mixture of 2-(2-(2-((2-(1-methyl-2,6-dioxopiperidin-3-yl)-1,3-dioxoisoindolin-4-yl) oxy)acetamido) ethoxy)ethyl methanesulfonate (30 mg, 0.059 mmol, 1.00 eq.), 8-cyclopentyl-2-((1-(piperazin-1-ylsulfonyl)piperidin-4-yl)amino)pyrido[2,3-d]pyrimidin-7 (8H)-one (28 mg, 0.061 mmol, 1.03 eq.), NaI (9 mg, 0.060 mmol, 1.02 eq.) and DIPEA (15 mg, 0.12 mmol, 2.03 eq.) in ACN (2.0 mL) was stirred at 80° C. overnight. The mixture was concentrated and purified by Prep-HPLC to get the title compound (1 mg, 1.9%) as a white solid. MS (ES, m/z): [M+1]⁺=877.3.

### Example 6

### Synthesis of N-(14-(4-((4-((8-cyclopentyl-7-oxo-7,8-dihydropyrido[2,3-d]pyrimidin-2-yl)amino)piperidin-1-yl)sulfonyl)piperazin-1-yl)-3,6,9,12-tetraoxatetradecyl)-2-((2-(2,6-dioxopiperidin-3-yl)-1,3-dioxoisoindolin-4-yl)oxy)acetamide

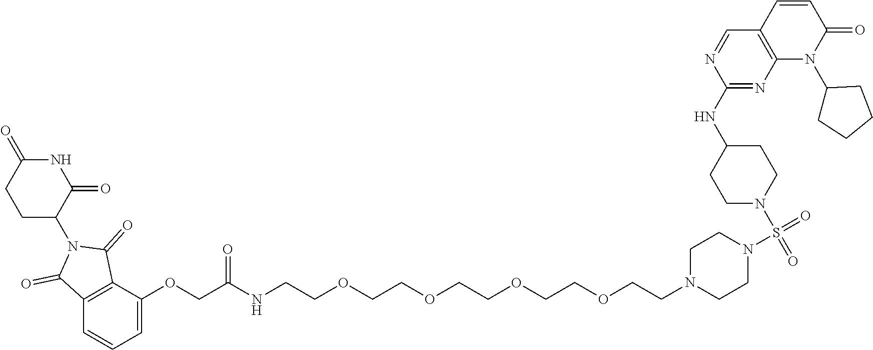 [SMILES: O=C(COc1cccc2c1C(=O)N(C1CCC(=O)NC1=O)C2=O)NCCOCCOCCOCCOCCN1CCN(S(=O)(=O)N2CCC(Nc3ncc4ccc(=O)n(C5CCCC5)c4n3)CC2)CC1]

### Step 1: 2-((2-(2,6-dioxopiperidin-3-yl)-1,3-dioxoisoindolin-4-yl)oxy)-N-(14-hydroxy-3,6,9,12-tetraoxatetradecyl)acetamide

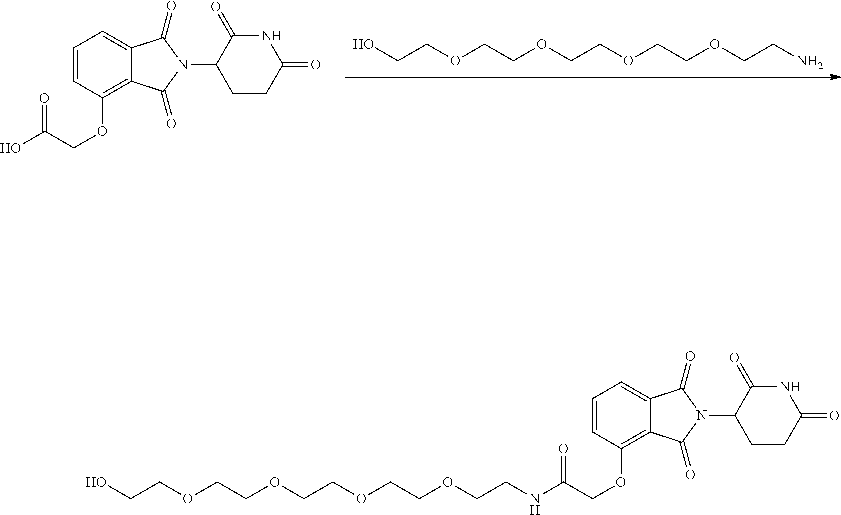 [SMILES: O=C(O)COc1cccc2c1C(=O)N(C1CCC(=O)NC1=O)C2=O ; NCCOCCOCCOCCOCCO ; O=C(COc1cccc2c1C(=O)N(C1CCC(=O)NC1=O)C2=O)NCCOCCOCCOCCOCCO]

A mixture of 2-((2-(2,6-dioxopiperidin-3-yl)-1,3-dioxoisoindolin-4-yl)oxy)acetic acid (300 mg, 0.90 mmol, 1.00 eq.), 14-amino-3,6,9,12-tetraoxatetradecan-1-ol (320 mg, 1.35 mmol, 1.50 eq.), HATU (513 mg, 1.35 mmol, 1.50 eq.) and DIPEA (348 mg, 2.69 mmol, 2.99 eq.) in DMF (6.0 mL) was stirred at 0° C. for 1 h. The reaction mixture was diluted with H₂O and extracted with DCM. The combined organic phase was washed with brine, dried over Na₂SO₄, concentrated to get crude title compound (800 mg) as a yellow oil, which was used for next step without further purification.

### Step 2: 1-((2-(2,6-dioxopiperidin-3-yl)-1,3-dioxoisoindolin-4-yl)oxy)-2-oxo-6,9,12,15-tetraoxa-3-azaheptadecan-17-yl methanesulfonate

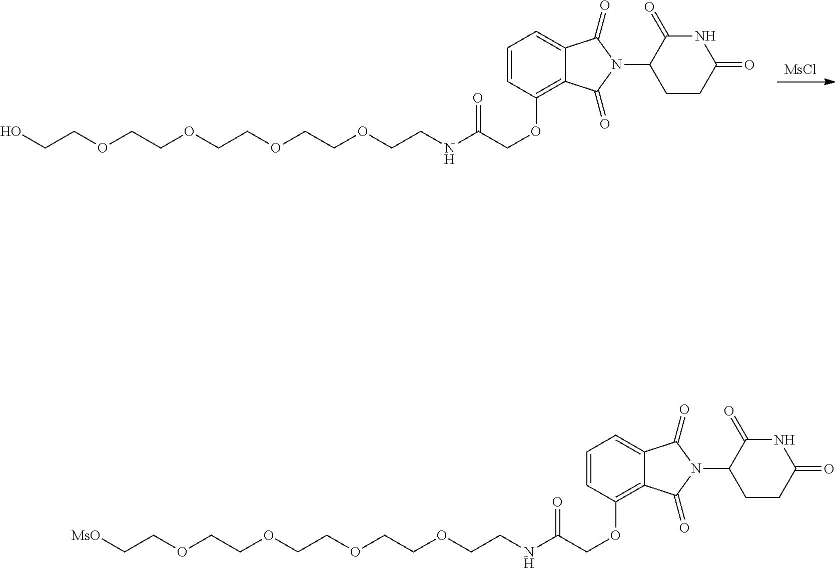 [SMILES: "MsCl" ; O=C(COc1cccc2c1C(=O)N(C1CCC(=O)NC1=O)C2=O)NCCOCCOCCOCCOCCO ; CS(=O)(=O)OCCOCCOCCOCCOCCNC(=O)COc1cccc2c1C(=O)N(C1CCC(=O)NC1=O)C2=O]

To a stirred solution of 2-((2-(2,6-dioxopiperidin-3-yl)-1,3-dioxoisoindolin-4-yl)oxy)-N-(14-hydroxy-3,6,9,12-tetraoxatetradecyl)acetamide (800 mg, 1.45 mmol, 1.00 eq.) and TEA (479 mg, 4.73 mmol, 326 eq.) in DCM (8.0 mL) was added MsCl (271 mg, 2.37 mmol, 1.63 eq.) slowly at 0° C. The reaction mixture was stirred at 0° C. for 1 h, diluted with H₂O and extracted with DCM. The combined organic phase was washed with brine, dried over Na₂SO₄, concentrated and purified by flash silica gel chromatography (DCM:MeOH=50:1) to get the title compound (200 mg, 35.6% over two steps) as a white solid.

### Step 3: N-(14-(4-((4-((8-cyclopentyl-7-oxo-7,8-dihydropyrido[2,3-d]pyrimidin-2-yl)amino)piperidin-1-yl)sulfonyl)piperazin-1-yl)-3,6,9,12-tetraoxatetradecyl)-2-((2-(2,6-dioxopiperidin-3-yl)-1,3-dioxoisoindolin-4-yl)oxy)acetamide

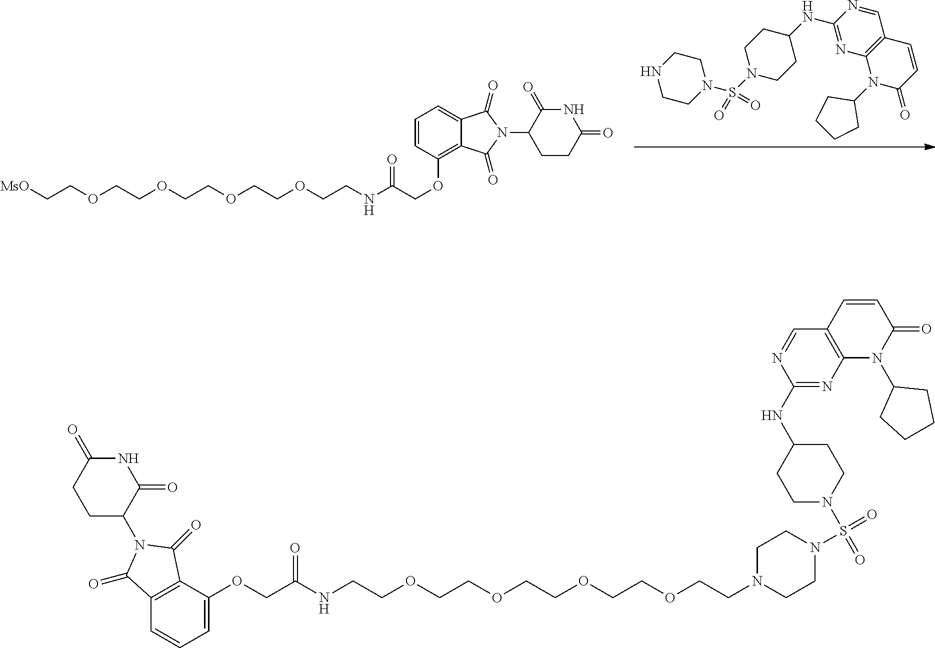 [SMILES: CS(=O)(=O)OCCOCCOCCOCCOCCNC(=O)COc1cccc2c1C(=O)N(C1CCC(=O)NC1=O)C2=O ; O=c1ccc2cnc(NC3CCN(S(=O)(=O)N4CCNCC4)CC3)nc2n1C1CCCC1 ; O=C(COc1cccc2c1C(=O)N(C1CCC(=O)NC1=O)C2=O)NCCOCCOCCOCCOCCN1CCN(S(=O)(=O)N2CCC(Nc3ncc4ccc(=O)n(C5CCCC5)c4n3)CC2)CC1]

A mixture of 1-((2-(2,6-dioxopiperidin-3-yl)-1,3-dioxoisoindolin-4-yl)oxy)-2-oxo-6,9,12,15-tetraoxa-3-azaheptadecan-17-yl methanesulfonate (82 mg, 0.13 mmol, 1.00 eq.), 8-cyclopentyl-2-((1-(piperazin-1-ylsulfonyl)piperidin-4-yl)amino)pyrido[2,3-d]pyrimidin-7 (8H)-one (60 mg, 0.13 mmol, 1.00 eq.), NaI (20 mg, 0.13 mmol, 1.00 eq.) and DIPEA (51 mg, 0.39 mmol, 3.00 eq) in ACN (5.0 mL) was stirred at 80° C. overnight. The mixture was concentrated and the residue was purified by Prep-HPLC to get the title compound (3 mg, 2.3%) as a white solid. MS (ES, m/z): [M+1]⁺=995.4.

### Example 7

### Synthesis of 5-(3-(4-((4-((8-cyclopentyl-7-oxo-7,8-dihydropyrido[2,3-d]pyrimidin-2-yl)amino)piperidin-1-yl)sulfonyl)phenoxy)azetidin-1-yl)-2-(2,6-dioxopiperidin-3-yl)isoindoline-1,3-dione

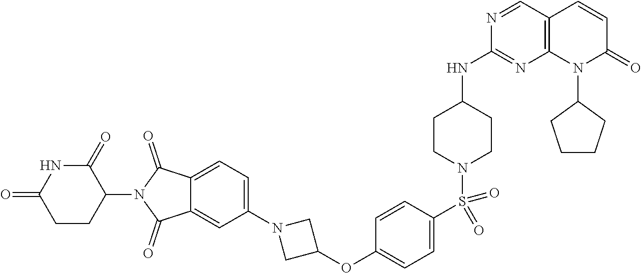 [SMILES: O=C1CCC(N2C(=O)c3ccc(N4CC(Oc5ccc(S(=O)(=O)N6CCC(Nc7ncc8ccc(=O)n(C9CCCC9)c8n7)CC6)cc5)C4)cc3C2=O)C(=O)N1]

### Step 1: tert-butyl (1-((4-fluorophenyl)sulfonyl)piperidin-4-yl)carbamate

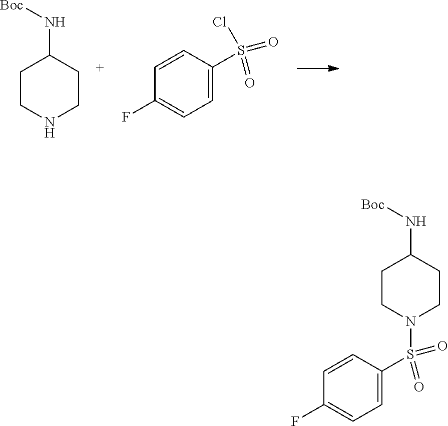 [SMILES: CC(C)(C)OC(=O)NC1CCNCC1 ; O=S(=O)(Cl)c1ccc(F)cc1 ; CC(C)(C)OC(=O)NC1CCN(S(=O)(=O)c2ccc(F)cc2)CC1]

To a stirred solution of tert-butyl piperidin-4-ylcarbamate (2.5 g, 12.48 mmol, 1.00 eq.) in DCM (10.0 mL) and TEA (5.2 mL) was added a solution of 4-fluorobenzenesulfonyl chloride (2.6 g, 13.36 mmol, 1.07 eq.) in DCM (10.0 mL) dropwise at 0° C. The resulting mixture was stirred at RT overnight, concentrated and diluted with DCM. The mixture was stirred at RT for 1 h and filtered to give the title compound (3.5 g, 78.2%) as a white solid.

### Step 2: tert-butyl (1-((4-((1-benzhydrylazetidin-3-yl)oxy)phenyl)sulfonyl)piperidin-4-yl)-carbamate

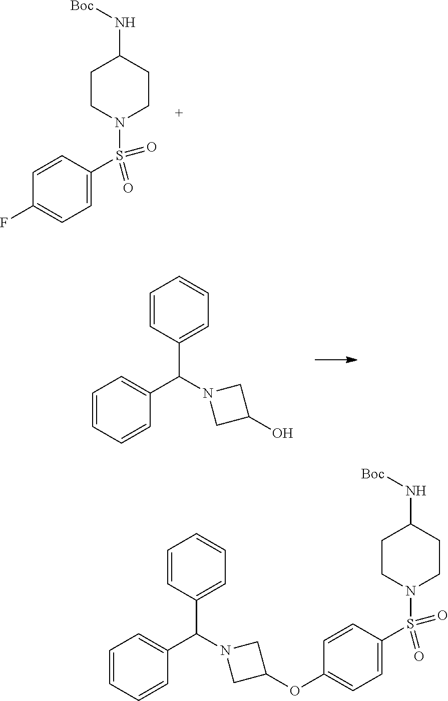 [SMILES: CC(C)(C)OC(=O)NC1CCN(S(=O)(=O)c2ccc(F)cc2)CC1 ; OC1CN(C(c2ccccc2)c2ccccc2)C1 ; CC(C)(C)OC(=O)NC1CCN(S(=O)(=O)c2ccc(OC3CN(C(c4ccccc4)c4ccccc4)C3)cc2)CC1]

To a stirred solution of 1-benzhydrylazetidin-3-ol (1.0 g, 4.18 mmol, 1.00 eq.) in THF (5.0 mL) was added NaH (60%, 251 mg, 6.28 mmol, 1.50 eq.) at 0° C. under N₂. The resulting mixture was stirred at RT for 15 min, then a solution of tert-butyl (1-((4-fluorophenyl)sulfonyl)piperidin-4-yl)carbamate (1.65 g, 4.60 mmol, 1.10 eq.) in THF (5.0 mL) was added slowly. The reaction mixture was stirred at RT overnight, diluted with H₂O, and then extracted with DCM. The combined organic layer was washed with aq. NaCl, dried over Na₂SO₄, filtered, and then concentrated. The crude was purified by silica gel flash column (PE:EA=3:1) to give the title compound (1.5 g, 62.2%) as a white solid.

### Step 3: tert-butyl (1-((4-(azetidin-3-yloxy)phenyl)sulfonyl)piperidin-4-yl)carbamate

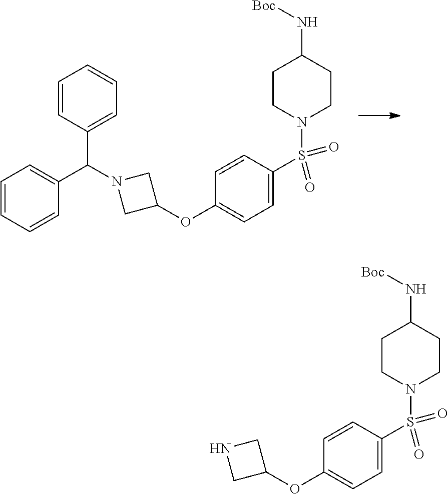 [SMILES: CC(C)(C)OC(=O)NC1CCN(S(=O)(=O)c2ccc(OC3CN(C(c4ccccc4)c4ccccc4)C3)cc2)CC1 ; CC(C)(C)OC(=O)NC1CCN(S(=O)(=O)c2ccc(OC3CNC3)cc2)CC1]

To a stirred solution of tert-butyl (1-((4-((1-benzhydrylazetidin-3-yl)oxy)phenyl)sulfonyl) piperidin-4-yl)carbamate (500 mg, 0.87 mmol, 1.00 eq.) in THF (20.0 mL) was added Pd(OH)₂ (300 mg, 20% on carbon) at room temperature. The resulting reaction mixture was stirred at 50° C. under H₂ (50 psi) overnight, cooled, filtrated, concentrated and purified by silica gel flash column (DCM:MeOH=10:1) to give the title compound (342 mg, 95.4%) as a white solid.

### Step 4: tert-butyl (1-((4-((1-(2-(2,6-dioxopiperidin-3-yl)-1,3-dioxoisoindolin-5-yl)azetidin-3-yl) oxy)phenyl)sulfonyl)piperidin-4-yl)carbamate

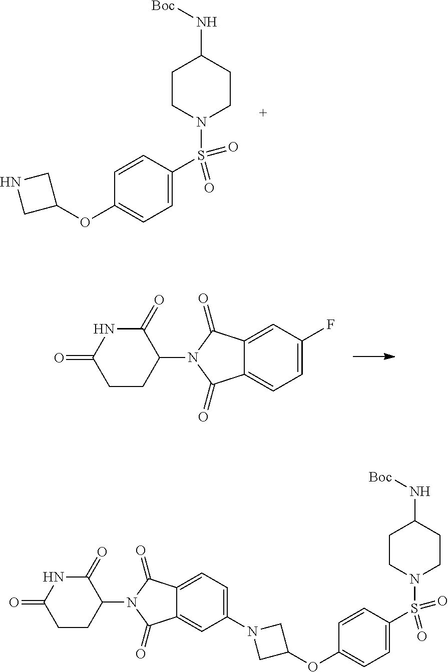 [SMILES: CC(C)(C)OC(=O)NC1CCN(S(=O)(=O)c2ccc(OC3CNC3)cc2)CC1 ; O=C1CCC(N2C(=O)c3ccc(F)cc3C2=O)C(=O)N1 ; CC(C)(C)OC(=O)NC1CCN(S(=O)(=O)c2ccc(OC3CN(c4ccc5c(c4)C(=O)N(C4CCC(=O)NC4=O)C5=O)C3)cc2)CC1]

To a stirred solution of tert-butyl (1-((4-(azetidin-3-yloxy)phenyl)sulfonyl)piperidin-4-yl) carbamate (100 mg, 0.24 mmol, 1.00 eq.) in NMP (1.5 mL) were added 2-(2,6-dioxopiperidin-3-yl)-5-fluoroisoindoline-1,3-dione (74 mg, 0.27 mmol, 1.13 eq.) and DIPEA (94 mg, 0.73 mmol, 3.04 eq.) at room temperature under N₂. The resulting mixture was stirred at 140° C. for 2 h under microwave irradiation. The reaction mixture was cooled, diluted with water, extracted with DCM, and then concentrated. The crude was purified by silica gel flash column (PE:EA=1:1) to give the title product (144 mg, 87.5%) as a yellow solid.

### Step 5: 5-(3-(4-((4-aminopiperidin-1-yl)sulfonyl)phenoxy)azetidin-1-yl)-2-(2,6-dioxopiperidin-3-yl) isoindoline-1,3-dione

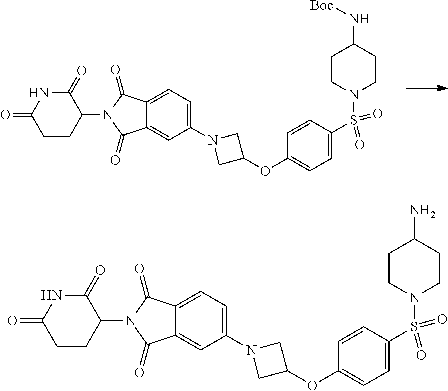 [SMILES: CC(C)(C)OC(=O)NC1CCN(S(=O)(=O)c2ccc(OC3CN(c4ccc5c(c4)C(=O)N(C4CCC(=O)NC4=O)C5=O)C3)cc2)CC1 ; NC1CCN(S(=O)(=O)c2ccc(OC3CN(c4ccc5c(c4)C(=O)N(C4CCC(=O)NC4=O)C5=O)C3)cc2)CC1]

To a stirred solution of tert-butyl (1-((4-((1-(2-(2,6-dioxopiperidin-3-yl)-1,3-dioxo-isoindolin-5-yl)azetidin-3-yl)oxy)phenyl)sulfonyl)piperidin-4-yl)carbamate (144 mg, 0.21 mmol, 1.00 eq.) in DCM (4.0 mL) was added TFA (1.0 mL) at room temperature. The resulting mixture was stirred at RT for 2 h, concentrated to give the title compound (130 mg, 100%) as a yellow oil, which was used for next step without further purification.

### Step 6: 5-(3-(4-((4-((8-cyclopentyl-7-oxo-7,8-dihydropyrido[2,3-d]pyrimidin-2-yl)amino)-piperidin-1-yl)sulfonyl)phenoxy)azetidin-1-yl)-2-(2,6-dioxopiperidin-3-yl)isoindoline-1,3-dione

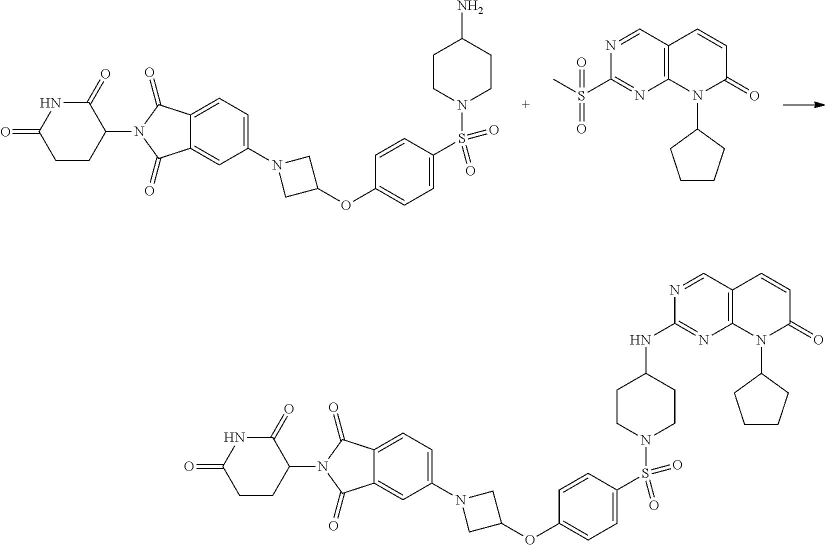 [SMILES: NC1CCN(S(=O)(=O)c2ccc(OC3CN(c4ccc5c(c4)C(=O)N(C4CCC(=O)NC4=O)C5=O)C3)cc2)CC1 ; CS(=O)(=O)c1ncc2ccc(=O)n(C3CCCC3)c2n1 ; O=C1CCC(N2C(=O)c3ccc(N4CC(Oc5ccc(S(=O)(=O)N6CCC(Nc7ncc8ccc(=O)n(C9CCCC9)c8n7)CC6)cc5)C4)cc3C2=O)C(=O)N1]

To a stirred solution of 8-cyclopentyl-2-(methylsulfonyl)pyrido[2,3-d]pyrimidin-7 (8H)-one (60 mg, 0.20 mmol, 1.00 eq.) in DMSO (2.0 mL) were added 5-(3-(4-((4-aminopiperidin-1-yl)sulfonyl)phenoxy)azetidin-1-yl)-2-(2,6-dioxopiperidin-3-yl)isoindoline-1,3-dione (128 mg, 0.23 mmol, 1.15 eq.) and DIPEA (79 mg, 0.61 mmol, 3.05 eq.) at room temperature. The resulting mixture was stirred at 65° C. overnight, cooled and purified by prep-HPLC to give the title compound (23 mg, 14.5%) as a yellow solid. MS (ES, m/z): [M+1]⁺=781.4.

### Example 8

### Synthesis of 5-((3-(4-((4-((8-cyclopentyl-7-oxo-7,8-dihydropyrido[2,3-d]pyrimidin-2-yl)-amino)piperidin-1-yl)sulfonyl)phenoxy)azetidin-1-yl)methyl)-2-(2,6-dioxopiperidin-3-yl)isoindoline-1,3-dione

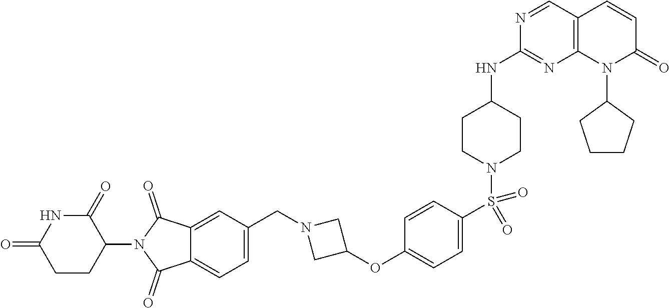 [SMILES: O=C1CCC(N2C(=O)c3ccc(CN4CC(Oc5ccc(S(=O)(=O)N6CCC(Nc7ncc8ccc(=O)n(C9CCCC9)c8n7)CC6)cc5)C4)cc3C2=O)C(=O)N1]

### Step 1: tert-butyl (1-((4-fluorophenyl)sulfonyl)piperidin-4-yl)carbamate

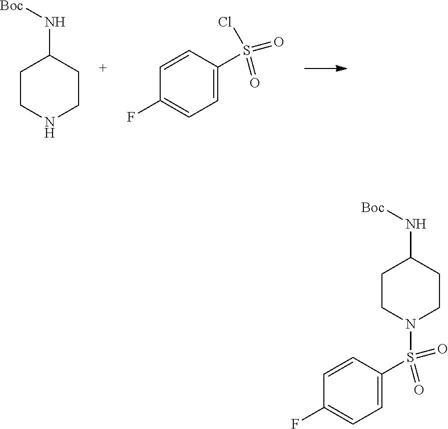 [SMILES: CC(C)(C)OC(=O)NC1CCNCC1 ; O=S(=O)(Cl)c1ccc(F)cc1 ; CC(C)(C)OC(=O)NC1CCN(S(=O)(=O)c2ccc(F)cc2)CC1]

To a stirred solution of tert-butyl piperidin-4-ylcarbamate (2.5 g, 12.48 mmol, 1.00 eq.) in DCM (10.0 mL) and TEA (5.2 mL) was added a solution of 4-fluorobenzenesulfonyl chloride (2.6 g, 13.36 mmol, 1.07 eq.) in DCM (10.0 mL) dropwise at 0° C. The resulting mixture was stirred at RT overnight, concentrated and diluted with DCM (20 mL). The mixture was stirred at RT for 1 h and filtered to give the title compound (3.5 g, 78.2%) as a white solid.

### Step 2: tert-butyl (1-((4-((1-benzhydrylazetidin-3-yl)oxy)phenyl)sulfonyl)piperidin-4-yl)-carbamate

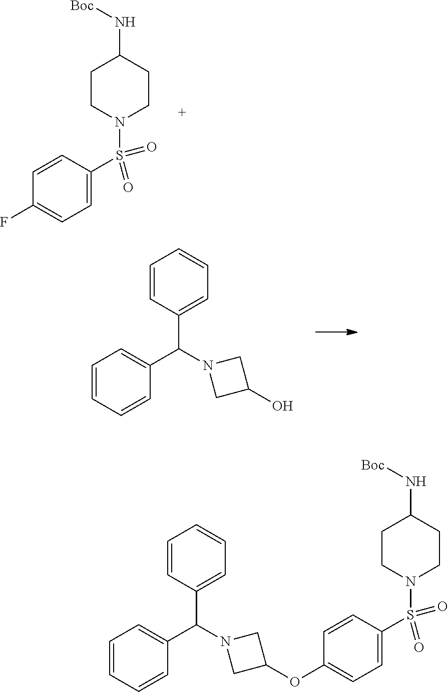 [SMILES: CC(C)(C)OC(=O)NC1CCN(S(=O)(=O)c2ccc(F)cc2)CC1 ; OC1CN(C(c2ccccc2)c2ccccc2)C1 ; CC(C)(C)OC(=O)NC1CCN(S(=O)(=O)c2ccc(OC3CN(C(c4ccccc4)c4ccccc4)C3)cc2)CC1]

To a stirred solution of 1-benzhydrylazetidin-3-ol (1.0 g, 4.18 mmol, 1.00 eq.) in THF (5.0 mL) was added NaH (60%, 251 mg, 6.28 mmol, 1.50 eq.) at 0° C. under N₂. The resulting mixture was stirred at RT for 15 min, then a solution of tert-butyl (1-((4-fluorophenyl)sulfonyl)piperidin-4-yl) carbamate (1.65 g, 4.60 mmol, 1.10 eq.) in THF (5.0 mL) was added slowly. The reaction mixture was stirred at RT overnight, diluted with H₂O, and then extracted with DCM. The combined organic layer was washed with aq. NaCl, dried over Na₂SO₄, filtered, and then concentrated. The crude was purified by silica gel flash column (PE:EA=3:1) to give the title compound (1.5 g, 62.2%) as a white solid.

### Step 3: tert-butyl (1-((4-(azetidin-3-yloxy)phenyl)sulfonyl)piperidin-4-yl)carbamate

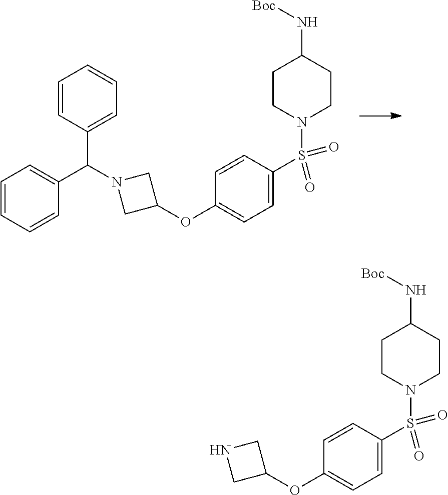 [SMILES: CC(C)(C)OC(=O)NC1CCN(S(=O)(=O)c2ccc(OC3CN(C(c4ccccc4)c4ccccc4)C3)cc2)CC1 ; CC(C)(C)OC(=O)NC1CCN(S(=O)(=O)c2ccc(OC3CNC3)cc2)CC1]

To a stirred solution of tert-butyl (1-((4-((1-benzhydrylazetidin-3-yl)oxy)phenyl)sulfonyl) piperidin-4-yl)carbamate (500 mg, 0.87 mmol, 1.00 eq.) in THF (20.0 mL) was added Pd(OH)₂ (300 mg, 20% on carbon) at room temperature. The resulting reaction mixture was stirred at 50° C. under H₂ (50 psi) overnight, cooled, filtrated, concentrated and purified by silica gel flash column (DCM:MeOH=10:1) to give the title compound (342 mg, 95.4%) as a white solid.

### Step 4: 5-(bromomethyl)-2-(2,6-dioxopiperidin-3-yl)isoindoline-1,3-dione

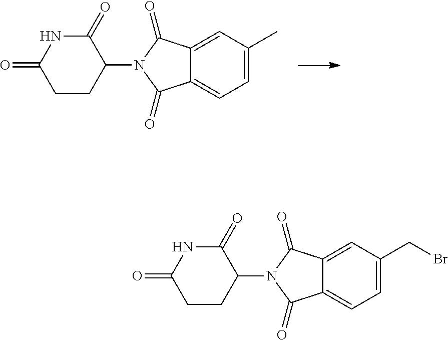 [SMILES: Cc1ccc2c(c1)C(=O)N(C1CCC(=O)NC1=O)C2=O ; O=C1CCC(N2C(=O)c3ccc(CBr)cc3C2=O)C(=O)N1]

To a stirred solution of 2-(2,6-dioxopiperidin-3-yl)-5-methylisoindoline-1,3-dione (272 mg, 1.00 mmol, 1.00 eq.) in MCCN (15.0 mL) were added NBS (196 mg, 1.10 mmol, 1.10 eq.) and AIBN (32.8 mg, 0.20 mmol, 0.20 eq.). The resulting mixture was stirred at 80° C. overnight under N2, cooled, and concentrated. Purification by flash column chromatography (EA:PE=0-100%) gave the title compound (256 mg, 73.0%) as a white solid.

### Step 5: tert-butyl (1-((4-((1-((2-(2,6-dioxopiperidin-3-yl)-1,3-dioxoisoindolin-5-yl)methyl) azetidin-3-yl)oxy)phenyl)sulfonyl)piperidin-4-yl)carbamate

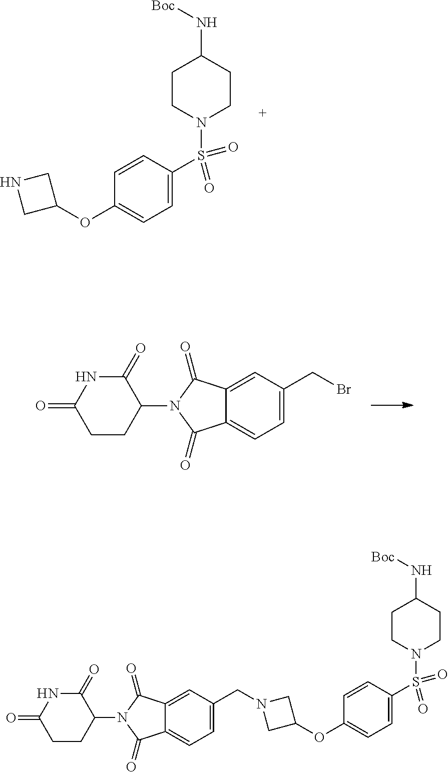 [SMILES: CC(C)(C)OC(=O)NC1CCN(S(=O)(=O)c2ccc(OC3CNC3)cc2)CC1 ; O=C1CCC(N2C(=O)c3ccc(CBr)cc3C2=O)C(=O)N1 ; CC(C)(C)OC(=O)NC1CCN(S(=O)(=O)c2ccc(OC3CN(Cc4ccc5c(c4)C(=O)N(C4CCC(=O)NC4=O)C5=O)C3)cc2)CC1]

A mixture of tert-butyl (1-((4-(azetidin-3-yloxy)phenyl)sulfonyl)piperidin-4-yl)carbamate (100 mg, 0.24 mmol, 1.00 eq., from Example 7, Step 3), 5-(bromomethyl)-2-(2,6-dioxopiperidin-3-yl)isoindoline-1,3-dione (111 mg, 0.32 mmol, 1.33 eq.) and K2CO₃ (67 mg, 0.48 mmol, 2.00 eq.) in MCCN (2.0 mL) was stirred at 80° C. overnight. The reaction mixture was cooled, concentrated and purified by silica gel flash column (DCM:MeOH=20:1) to give the title compound (33 mg, 18.3%) as a white solid.

### Step 6: 5-((3-(4-((4-aminopiperidin-1-yl)sulfonyl)phenoxy)azetidin-1-yl)methyl)-2-(2,6-dioxo-piperidin-3-yl)isoindoline-1,3-dione

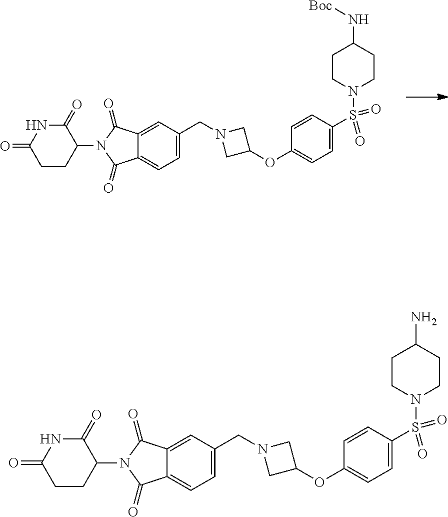 [SMILES: CC(C)(C)OC(=O)NC1CCN(S(=O)(=O)c2ccc(OC3CN(Cc4ccc5c(c4)C(=O)N(C4CCC(=O)NC4=O)C5=O)C3)cc2)CC1 ; NC1CCN(S(=O)(=O)c2ccc(OC3CN(Cc4ccc5c(c4)C(=O)N(C4CCC(=O)NC4=O)C5=O)C3)cc2)CC1]

To a solution of tert-butyl (1-((4-((1-((2-(2,6-dioxopiperidin-3-yl)-1,3-dioxoisoindolin-5-yl) methyl)azetidin-3-yl)oxy)phenyl)sulfonyl)piperidin-4-yl)carbamate (33 mg, 0.048 mmol, 1.00 eq.) in DCM (4.0 mL) was added TFA (1.0 mL) at room temperature. The resulting reaction mixture was stirred at RT for 3 h, and then concentrated to give the title compound (30 mg, 100%) as a yellow solid, which was used for next step without further purification.

### Step 7: 5-((3-(4-((4-((8-cyclopentyl-7-oxo-7,8-dihydropyrido[2,3-d]pyrimidin-2-yl)amino)-piperidin-1-yl)sulfonyl)phenoxy)azetidin-1-yl)methyl)-2-(2,6-dioxopiperidin-3-yl)isoindoline-1,3-dione

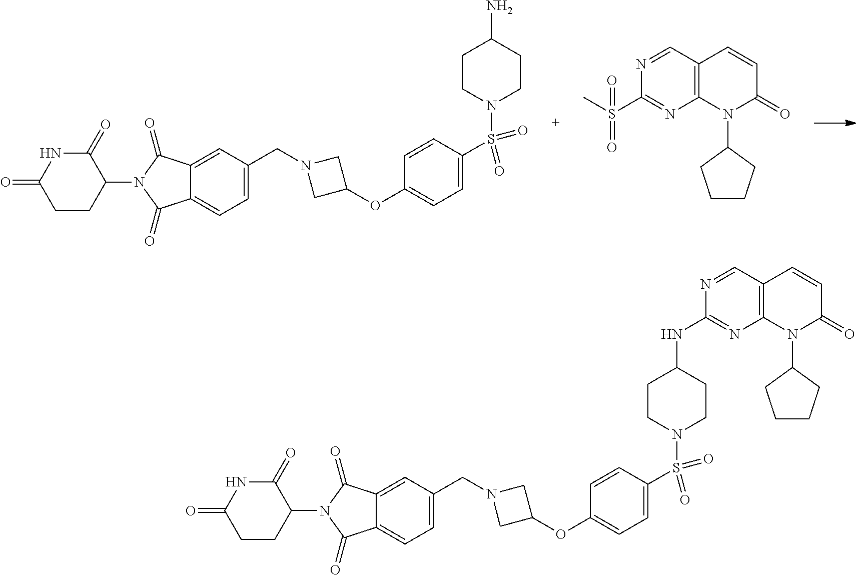 [SMILES: NC1CCN(S(=O)(=O)c2ccc(OC3CN(Cc4ccc5c(c4)C(=O)N(C4CCC(=O)NC4=O)C5=O)C3)cc2)CC1 ; CS(=O)(=O)c1ncc2ccc(=O)n(C3CCCC3)c2n1 ; O=C1CCC(N2C(=O)c3ccc(CN4CC(Oc5ccc(S(=O)(=O)N6CCC(Nc7ncc8ccc(=O)n(C9CCCC9)c8n7)CC6)cc5)C4)cc3C2=O)C(=O)N1]

To a stirred solution of 8-cyclopentyl-2-(methylsulfonyl)pyrido[2,3-d]pyrimidin-7 (8H)-one (17 mg, 0.058 mmol, 1.12 eq.) in DMSO (1.0 mL) were added DIPEA (20 mg, 0.15 mmol, 2.88 eq.) and 5-((3-(4-((4-aminopiperidin-1-yl)sulfonyl)phenoxy)azetidin-1-yl)methyl)-2-(2,6-dioxopiperidin-3-yl)isoindoline-1,3-dione (30 mg, 0.052 mmol, 1.00 eq.) at room temperature. The resulting reaction mixture was stirred at 65° C. overnight, cooled and purified by prep-HPLC to give title compound (2.1 mg, 5.0%) as a white solid. MS (ES, m/z): [M+1]⁺=795.4.

### Example 9

### Synthesis of 4-((8-cyclopentyl-7-oxo-7,8-dihydropyrido[2,3-d]pyrimidin-2-yl)amino)-N-(2-(2-(2-((2-(2,6-dioxopiperidin-3-yl)-1,3-dioxoisoindolin-4-yl)amino)ethoxy)ethoxy)ethyl)piperidine-1-sulfonamide

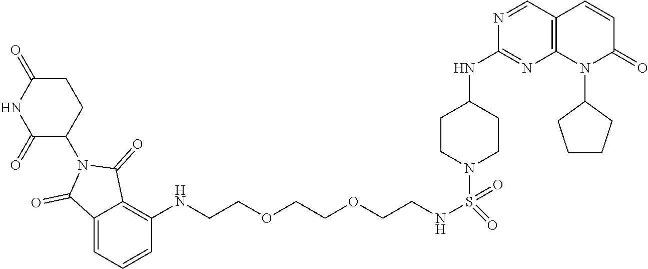 [SMILES: O=C1CCC(N2C(=O)c3cccc(NCCOCCOCCNS(=O)(=O)N4CCC(Nc5ncc6ccc(=O)n(C7CCCC7)c6n5)CC4)c3C2=O)C(=O)N1]

### Step 1: tert-butyl (2-(2-(2-((2-(2,6-dioxopiperidin-3-yl)-1,3-dioxoisoindolin-4-yl)amino)ethoxy)-ethoxy)ethyl)carbamate

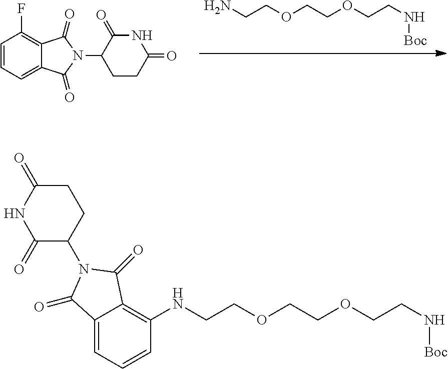 [SMILES: O=C1CCC(N2C(=O)c3cccc(F)c3C2=O)C(=O)N1 ; CC(C)(C)OC(=O)NCCOCCOCCN ; CC(C)(C)OC(=O)NCCOCCOCCNc1cccc2c1C(=O)N(C1CCC(=O)NC1=O)C2=O]

To a stirred solution of 2-(2,6-dioxopiperidin-3-yl)-4-fluoroisoindoline-1,3-dione (100 mg, 0.36 mmol, 1.10 eq.) and tert-butyl (2-(2-(2-aminoethoxy)ethoxy)ethyl)carbamate (81.7 mg, 0.33 mmol, 1.00 eq.) in NMP (1.5 mL) was added DIPEA (127 mg, 0.98 mmol, 2.97 eq.). The resulting mixture was stirred at 140° C. under microwave for 2 h. The reaction mixture was cooled and diluted with ethyl acetate, and then washed with water, brine, dried over Na₂SO₄, and concentrated. Purification of the crude material by silica gel chromatography (EA:PE=1:3) to give the title compound (180 mg, 100%) as a yellow oil.

### Step 2: 4-((2-(2-(2-aminoethoxy)ethoxy)ethyl)amino)-2-(2,6-dioxopiperidin-3-yl)isoindoline-1,3-dione

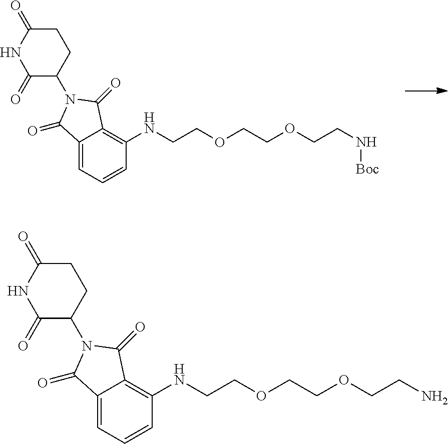 [SMILES: CC(C)(C)OC(=O)NCCOCCOCCNc1cccc2c1C(=O)N(C1CCC(=O)NC1=O)C2=O ; NCCOCCOCCNc1cccc2c1C(=O)N(C1CCC(=O)NC1=O)C2=O]

To a stirred solution of tert-butyl (2-(2-(2-((2-(2,6-dioxopiperidin-3-yl)-1,3-dioxo-isoindolin-4-yl)amino)ethoxy)ethoxy)ethyl)carbamate (180 mg, 0.36 mmol, 1.00 eq.) in DCM (2.0 mL) was added TFA (0.5 mL). The resulting mixture was stirred at RT for 2 h, and then concentrated to give title compound (144 mg, crude) as a yellow oil, which was used for next step without further purification.

### Step 3: tert-butyl (1-(chlorosulfonyl)piperidin-4-yl)carbamate

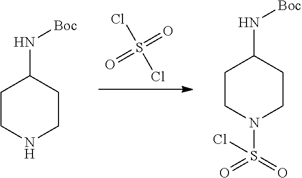 [SMILES: CC(C)(C)OC(=O)NC1CCNCC1 ; O=S(=O)(Cl)Cl ; CC(C)(C)OC(=O)NC1CCN(S(=O)(=O)Cl)CC1]

To a stirred solution of tert-butyl piperidin-4-ylcarbamate (100 mg, 0.50 mmol, 1.00 eq.) and TEA (76 mg, 0.75 mmol, 1.50 eq.) in DCM (2.0 mL) was added sulfuryl dichloride (81 mg, 0.60 mmol, 1.20 eq.) at 0° C. The resulting mixture was stirred at 0° C. for 3 h, diluted with water, and then extracted with DCM. The organic layer was washed with brine, dried over Na₂SO₄, and then concentrated to give the title compound (150 mg, crude) as a white solid, which was used for next step directly.

### Step 4: tert-butyl (1-(N-(2-(2-(2-((2-(2,6-dioxopiperidin-3-yl)-1,3-dioxoisoindolin-4-yl)amino)-ethoxy)ethoxy)ethyl)sulfamoyl)piperidin-4-yl)carbamate

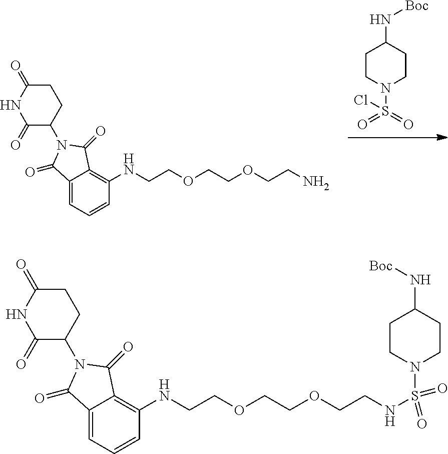 [SMILES: NCCOCCOCCNc1cccc2c1C(=O)N(C1CCC(=O)NC1=O)C2=O ; CC(C)(C)OC(=O)NC1CCN(S(=O)(=O)Cl)CC1 ; CC(C)(C)OC(=O)NC1CCN(S(=O)(=O)NCCOCCOCCNc2cccc3c2C(=O)N(C2CCC(=O)NC2=O)C3=O)CC1]

To a stirred solution of 4-((2-(2-(2-aminoethoxy)ethoxy)ethyl)amino)-2-(2,6-dioxo-piperidin-3-yl)isoindoline-1,3-dione (70 mg, 0.17 mmol, 1.00 eq.) and tert-butyl (1-(chloro-sulfonyl)piperidin-4-yl)carbamate (51.9 mg, 0.17 mmol, 1.00 eq.) in DCM (2.0 mL) was added TEA (52.4 mg, 0.52 mmol, 3.00 eq.). The resulting mixture was stirred at 35° C. overnight, and then concentrated. The residue was purified by silica gel chromatography (DCM:MeOH=30:1) to give the title compound (60 mg, 52.9%) as a yellow oil.

### Step 5: 4-amino-N-(2-(2-(2-((2-(2,6-dioxopiperidin-3-yl)-1,3-dioxoisoindolin-4-yl)amino)-ethoxy)ethoxy)ethyl)piperidine-1-sulfonamide

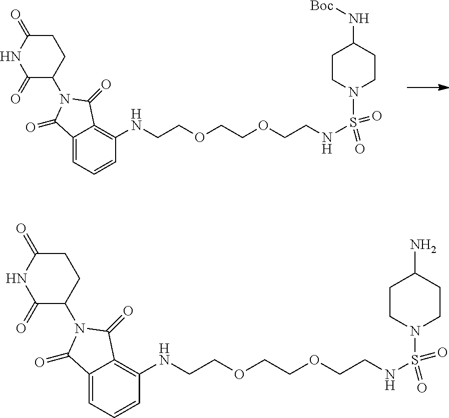 [SMILES: CC(C)(C)OC(=O)NC1CCN(S(=O)(=O)NCCOCCOCCNc2cccc3c2C(=O)N(C2CCC(=O)NC2=O)C3=O)CC1 ; NC1CCN(S(=O)(=O)NCCOCCOCCNc2cccc3c2C(=O)N(C2CCC(=O)NC2=O)C3=O)CC1]

To a stirred solution of tert-butyl (1-(N-(2-(2-(2-((2-(2,6-dioxopiperidin-3-yl)-1,3-dioxoisoindolin-4-yl)amino)ethoxy)ethoxy)ethyl)sulfamoyl)piperidin-4-yl)carbamate (60 mg, 0.090 mmol, 1.00 eq.) in DCM (2.0 mL) was added TFA (0.5 mL). The resulting mixture was stirred at RT for 2 h, and then concentrated to give the title compound (50.9 mg, crude) as a yellow oil, which was used for next step directly.

### Step 6: 4-((8-cyclopentyl-7-oxo-7,8-dihydropyrido[2,3-d]pyrimidin-2-yl)amino)-N-(2-(2-(2-((2-(2,6-dioxopiperidin-3-yl)-1,3-dioxoisoindolin-4-yl)amino)ethoxy)ethoxy)ethyl)piperidine-1-sulfonamide

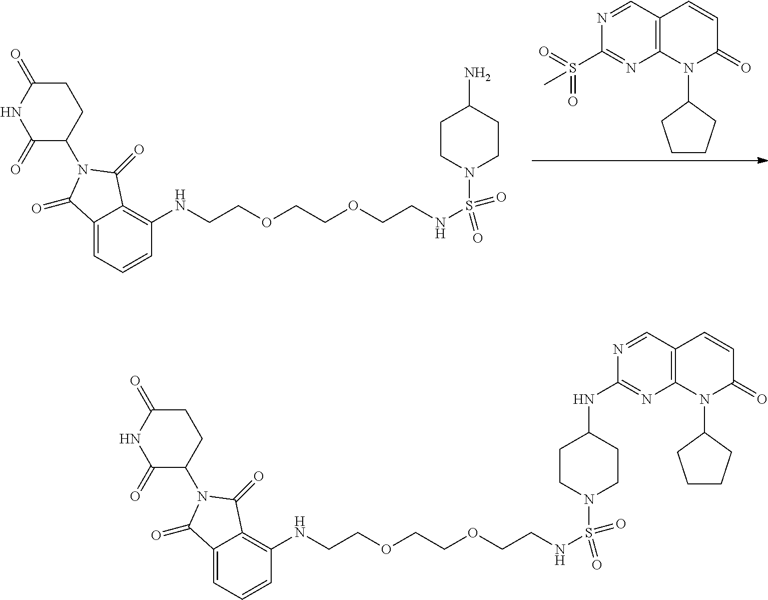 [SMILES: NC1CCN(S(=O)(=O)NCCOCCOCCNc2cccc3c2C(=O)N(C2CCC(=O)NC2=O)C3=O)CC1 ; CS(=O)(=O)c1ncc2ccc(=O)n(C3CCCC3)c2n1 ; O=C1CCC(N2C(=O)c3cccc(NCCOCCOCCNS(=O)(=O)N4CCC(Nc5ncc6ccc(=O)n(C7CCCC7)c6n5)CC4)c3C2=O)C(=O)N1]

To a stirred solution of 4-amino-N-(2-(2-(2-((2-(2,6-dioxopiperidin-3-yl)-1,3-dioxoisoindolin-4-yl)amino)ethoxy)ethoxy)ethyl)piperidine-1-sulfonamide (50 mg, 0.088 mmol, 1.00 eq.) and 8-cyclopentyl-2-(methylsulfonyl)pyrido[2,3-d]pyrimidin-7 (8H)-one (33.6 mg, 0.11 mmol, 1.25 eq.) in DMSO (2.0 mL) was added DIPEA (68 mg, 0.53 mmol, 6.00 eq.) at RT. The resulting mixture was stirred at 65° C. under N2 overnight, cooled, diluted with ethyl acetate, and then washed with water. The organic layer was washed with brine, dried over Na₂SO₄, concentrated and purified by prep-HPLC to give the title compound (12.2 mg, 18.2%) as a white solid. MS (ES, m/z): [M+1]⁺=780.4.

### Example 10

### Synthesis of 4-((8-cyclopentyl-7-oxo-7,8-dihydropyrido[2,3-d]pyrimidin-2-yl)amino)-N-(3-(3-(2-(2,6-dioxopiperidin-3-yl)-1,3-dioxoisoindolin-4-yl)propoxy)propyl)-N-methylpiperidine-1-sulfonamide

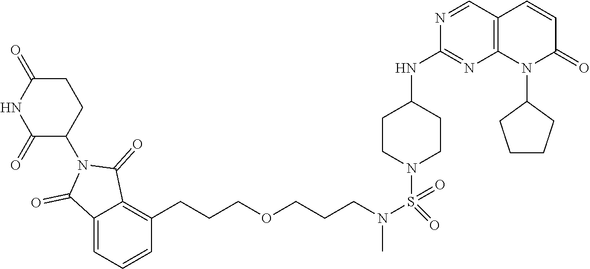 [SMILES: CN(CCCOCCCc1cccc2c1C(=O)N(C1CCC(=O)NC1=O)C2=O)S(=O)(=O)N1CCC(Nc2ncc3ccc(=O)n(C4CCCC4)c3n2)CC1]

### Step 1: 4-bromo-2-(2,6-dioxopiperidin-3-yl)isoindoline-1,3-dione

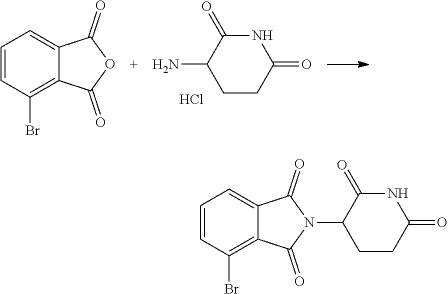 [SMILES: O=C1OC(=O)c2c(Br)cccc21 ; NC1CCC(=O)NC1=O ; Cl ; O=C1CCC(N2C(=O)c3cccc(Br)c3C2=O)C(=O)N1]

A mixture of 4-bromoisobenzofuran-1,3-dione (22.8 g, 100.44 mmol, 1.00 eq.), 3-aminopiperidine-2,6-dione (18.0 g, 109.36 mmol, HCl, 1.09 eq.) and KOAc (29.4 g, 299.54 mmol, 2.98 eq.) in HOAc (200.0 mL) was stirred at 90° C. for 16 h. The reaction mixture was cooled, diluted with ice water and then stirred at 0° C. for 1 h. The mixture was filtered and the filter cake was dried in vacuo to give the title compound (30 g, 88.6%) as gray solid.

### Step 2: tert-butyl methyl(3-(prop-2-yn-1-yloxy)propyl)carbamate

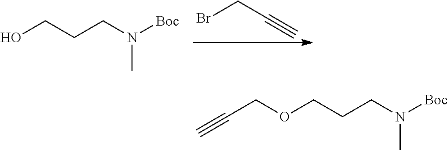

To a stirred mixture of tert-butyl (3-hydroxypropyl)(methyl)carbamate (3.0 g, 15.85 mmol, 1.00 eq.) in DCM (50.0 mL) was added 3-bromoprop-1-yne (3.0 g, 25.22 mmol, 1.59 eq.), 40% aqueous NaOH (30.0 mL) and tetrabutylammonium hydrogen sulfate (270 mg, 0.80 mmol, 0.050 eq.). The resulting mixture was stirred at RT overnight under N₂, diluted with water, and then extracted with DCM. The organic layer was washed with water, brine, dried over Na₂SO₄, concentrated and then purified by flash column chromatography (EA:PE=0 to 100%) to give the title compound (1.4 g, 38.9%) as a yellow oil.

### Step 3: tert-butyl (3-((3-(2-(2,6-dioxopiperidin-3-yl)-1,3-dioxoisoindolin-4-yl)prop-2-yn-1-yl)oxy)propyl)(methyl)carbamate

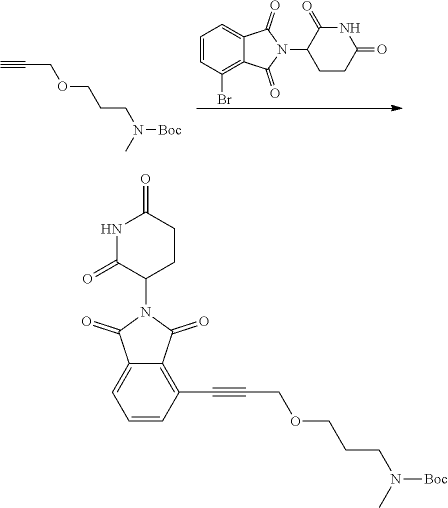

To a stirred solution of 4-bromo-2-(2,6-dioxopiperidin-3-yl)isoindoline-1,3-dione (1.38 g, 4.09 mmol, 1.00 eq.) in DMF (15.0 mL) was added tert-butyl methyl(3-(prop-2-yn-1-yloxy)-propyl)carbamate (1.4 g, 6.16 mmol, 1.51 eq.), CuI (78 mg, 0.41 mmol, 0.10 eq.), TEA (7.5 g, 74.12 mmol, 18.12 eq.) and Pd(PPh₃)₂Cl₂ (288 mg, 0.41 mmol, 0.10 eq.). The resulting mixture was stirred at 80° C. for 2 h under N2, cooled, diluted with water and then extracted with ethyl acetate. The organic layer was washed with water, brine, dried over Na₂SO₄, and concentrated. Purification by flash column chromatography (EA:PE=0 to 100%) to give the title compound (1.86 g, 94.1%) as a yellow oil.

### Step 4: tert-butyl (3-(3-(2-(2,6-dioxopiperidin-3-yl)-1,3-dioxoisoindolin-4-yl)propoxy)propyl)-(methyl)carbamate

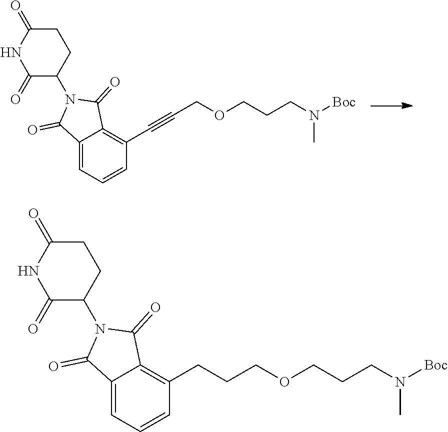 [SMILES: CN(CCCOCC#Cc1cccc2c1C(=O)N(C1CCC(=O)NC1=O)C2=O)C(=O)OC(C)(C)C ; CN(CCCOCCCc1cccc2c1C(=O)N(C1CCC(=O)NC1=O)C2=O)C(=O)OC(C)(C)C]

To a stirred solution of tert-butyl (3-((3-(2-(2,6-dioxopiperidin-3-yl)-1,3-dioxoisoindolin-4-yl)prop-2-yn-1-yl)oxy)propyl)(methyl)carbamate (1.86 g, 3.85 mmol, 1.00 eq.) in THF (50.0 mL) was added Pd(OH)2/C (0.93 g, 50% w/w). The resulting mixture was stirred at RT overnight under H₂ atmosphere. The reaction mixture was filtered, concentrated and purified by flash chromatography (EA:PE=0 to 100%) to give the title compound (1.45 g, 77.1%) as a yellow oil.

### Step 5: 2-(2,6-dioxopiperidin-3-yl)-4-(3-(3-(methylamino)propoxy)propyl)isoindoline-1,3-dione

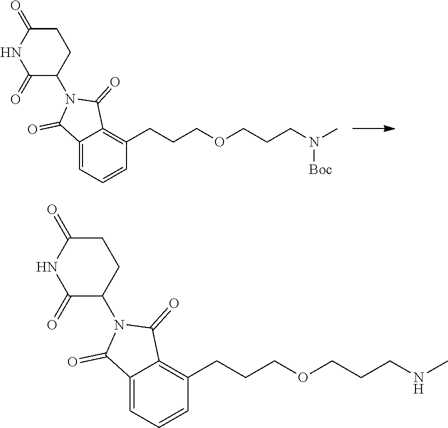 [SMILES: CN(CCCOCCCc1cccc2c1C(=O)N(C1CCC(=O)NC1=O)C2=O)C(=O)OC(C)(C)C ; CNCCCOCCCc1cccc2c1C(=O)N(C1CCC(=O)NC1=O)C2=O]

To a stirred solution of tert-butyl (3-(3-(2-(2,6-dioxopiperidin-3-yl)-1,3-dioxoisoindolin-4-yl)propoxy)propyl)(methyl)carbamate (1.45 g, 2.97 mmol, 1.00 eq.) in DCM (10.0 mL) was added TFA (1.0 mL). The resulting mixture was stirred at RT for 2 h under N₂, concentrated and adjusted pH to 9 using aqueous Na₂CO₃, and then extracted with DCM. The organic layer was washed with water, brine, dried over Na₂SO₄, and then concentrated to give the title compound (1.15 g, crude) as a yellow oil, which was used for next step without further purification.

### Step 6: tert-butyl (1-(N-(3-(3-(2-(2,6-dioxopiperidin-3-yl)-1,3-dioxoisoindolin-4-yl)propoxy)-propyl)-N-methylsulfamoyl)piperidin-4-yl)carbamate

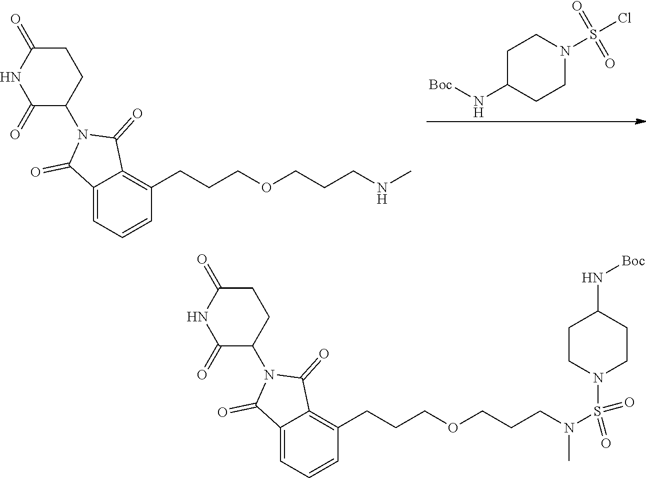 [SMILES: CNCCCOCCCc1cccc2c1C(=O)N(C1CCC(=O)NC1=O)C2=O ; CC(C)(C)OC(=O)NC1CCN(S(=O)(=O)Cl)CC1 ; CN(CCCOCCCc1cccc2c1C(=O)N(C1CCC(=O)NC1=O)C2=O)S(=O)(=O)N1CCC(NC(=O)OC(C)(C)C)CC1]

To a stirred solution of 2-(2,6-dioxopiperidin-3-yl)-4-(3-(3-(methylamino)propoxy)-propyl)isoindoline-1,3-dione (150 mg, 0.39 mmol, 1.00 eq.) in DCM (2.0 mL) was added tert-butyl (1-(chlorosulfonyl)piperidin-4-yl)carbamate (173 mg, 0.58 mmol, 1.49 eq.) and TEA (118 mg, 1.17 mmol, 3.00 eq.). The resulting mixture was stirred at 40° C. overnight under N2, cooled, diluted with water and then extracted with ethyl acetate. The organic layer was washed with water, brine, dried over Na₂S04, concentrated to give the title compound (200 mg, 79.5%) as a yellow solid.

### Step 7: 4-amino-N-(3-(3-(2-(2,6-dioxopiperidin-3-yl)-1,3-dioxoisoindolin-4-yl)propoxy)propyl)-N-methylpiperidine-1-sulfonamide

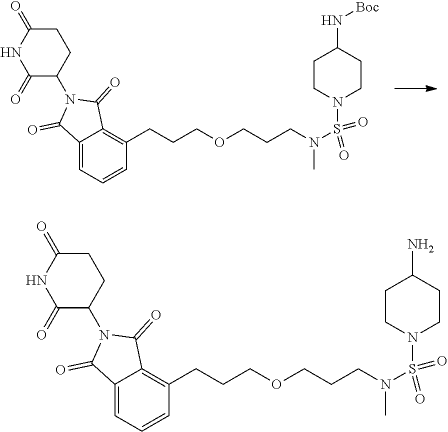 [SMILES: CN(CCCOCCCc1cccc2c1C(=O)N(C1CCC(=O)NC1=O)C2=O)S(=O)(=O)N1CCC(NC(=O)OC(C)(C)C)CC1 ; CN(CCCOCCCc1cccc2c1C(=O)N(C1CCC(=O)NC1=O)C2=O)S(=O)(=O)N1CCC(N)CC1]

To a stirred solution of tert-butyl (1-(N-(3-(3-(2-(2,6-dioxopiperidin-3-yl)-1,3-dioxoisoindolin-4-yl)propoxy)propyl)-N-methylsulfamoyl)piperidin-4-yl)carbamate (200 mg, 0.31 mmol, 1.00 eq.) in DCM (2.0 mL) was added TFA (0.5 mL). The resulting mixture was stirred at RT for 3 h under N₂, concentrated and adjusted pH to 9 using aqueous Na₂CO₃, and then extracted with DCM. The organic layer was washed with water, brine, dried over Na₂SO₄, concentrated to give the title compound (169 g, crude) as a yellow oil, which was used for next step without further purification.

### Step 8: 4-((8-cyclopentyl-7-oxo-7,8-dihydropyrido[2,3-d]pyrimidin-2-yl)amino)-N-(3-(3-(2-(2,6-dioxopiperidin-3-yl)-1,3-dioxoisoindolin-4-yl)propoxy)propyl)-N-methylpiperidine-1-sulfonamide

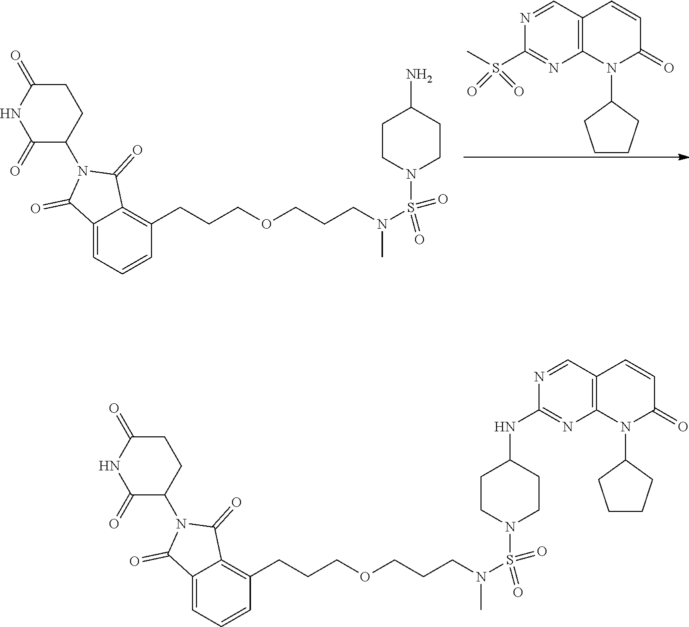

To a stirred solution of 8-cyclopentyl-2-(methylsulfonyl)pyrido[2,3-d]pyrimidin-7 (8H)-one (80 mg, 0.27 mmol, 1.00 eq.) in DMSO (3.0 mL) was added 4-amino-N-(3-(3-(2-(2,6-dioxopiperidin-3-yl)-1,3-dioxoisoindolin-4-yl)propoxy)propyl)-N-methylpiperidine-1-sulfonamide (168 mg, 0.31 mmol, 1.15 eq.) and DIPEA (106 mg, 0.82 mmol, 3.04 eq.). The resulting mixture was stirred at 65° C. overnight under N2, cooled, diluted with water and then extracted with ethyl acetate. The organic layer was washed with water, brine, dried over Na₂SO₄, concentrated and purified by prep-TLC (DCM:EA=1:1) to give the title compound (15.9 mg, 7.8%) as an off-white solid. MS (ES, m/z): [M+1]⁺=763.2.

### Example 11

### Synthesis of 4-((8-cyclopentyl-7-oxo-7,8-dihydropyrido[2,3-d]pyrimidin-2-yl)amino)-N-(2-(2-(3-(2-(2,6-dioxopiperidin-3-yl)-1,3-dioxoisoindolin-4-yl)propoxy)ethoxy)ethyl)piperidine-1-sulfonamide

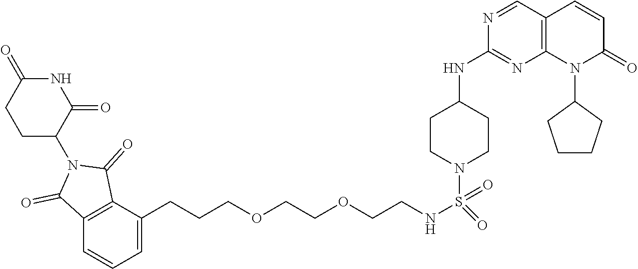 [SMILES: O=C1CCC(N2C(=O)c3cccc(CCCOCCOCCNS(=O)(=O)N4CCC(Nc5ncc6ccc(=O)n(C7CCCC7)c6n5)CC4)c3C2=O)C(=O)N1]

### Step 1: tert-butyl (2-(2-(prop-2-yn-1-yloxy)ethoxy)ethyl)carbamate

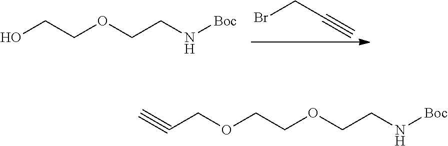

To a stirred solution of tert-butyl (2-(2-hydroxyethoxy)ethyl)carbamate (4.1 g, 19.98 mmol, 1.00 eq.) in THF (50.0 mL) was added NaH (1.2 g, 60%, 30.00 mmol, 1.50 eq.) in portions at 0° C. After stirring for 1 h, 3-bromoprop-1-yne (2.83 g, 23.79 mmol, 1.19 eq.) was added at 0° C. The reaction mixture was warmed to RT and stirred for 16 h, poured into water and extracted with DCM. The organic layer was washed with water, brine, dried over Na₂SO₄, concentrated and purified by flash column chromatography (EA:PE=0 to 100%) to give the title compound (1.7 g, 34.9%) as a yellow oil.

### Step 2: tert-butyl (2-(2-((3-(2-(2,6-dioxopiperidin-3-yl)-1,3-dioxoisoindolin-4-yl)prop-2-yn-1-yl)oxy)ethoxy)ethyl)carbamate

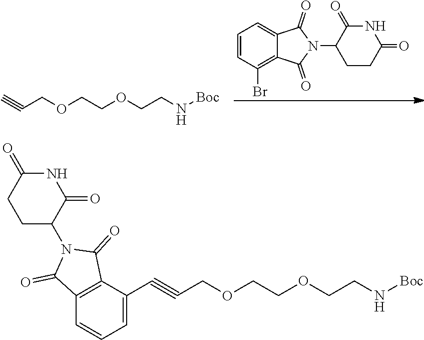 [SMILES: O=C1CCC(N2C(=O)c3cccc(Br)c3C2=O)C(=O)N1 ; C#CCOCCOCCNC(=O)OC(C)(C)C ; CC(C)(C)OC(=O)NCCOCCOCC#Cc1cccc2c1C(=O)N(C1CCC(=O)NC1=O)C2=O]

Proceeding analogously as described in Example 10, Step 3 above, but using 4-bromo-2-(2,6-dioxopiperidin-3-yl)isoindoline-1,3-dione and tert-butyl (2-(2-(prop-2-yn-1-yloxy)ethoxy)ethyl)carbamate in DMF provided the title compound.

### Step 3: tert-butyl (2-(2-(3-(2-(2,6-dioxopiperidin-3-yl)-1,3-dioxoisoindolin-4-yl)propoxy)-ethoxy)ethyl)carbamate

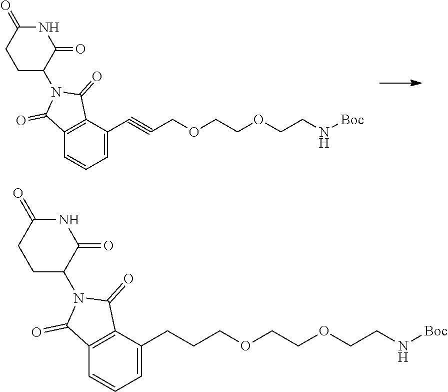 [SMILES: CC(C)(C)OC(=O)NCCOCCOCC#Cc1cccc2c1C(=O)N(C1CCC(=O)NC1=O)C2=O ; CC(C)(C)OC(=O)NCCOCCOCCCc1cccc2c1C(=O)N(C1CCC(=O)NC1=O)C2=O]

Proceeding analogously as described in Example 10, Step 4 above, but using tert-butyl (2-(2-((3-(2-(2,6-dioxopiperidin-3-yl)-1,3-dioxoisoindolin-4-yl)prop-2-yn-1-yl)oxy)ethoxy)ethyl)carbamate provided the title compound.

### Step 4: 4-(3-(2-(2-aminoethoxy)ethoxy)propyl)-2-(2,6-dioxopiperidin-3-yl)isoindoline-1,3-dione

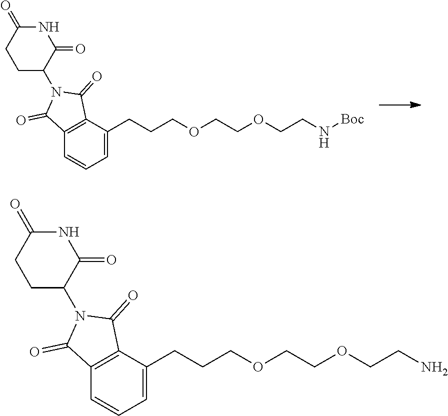 [SMILES: CC(C)(C)OC(=O)NCCOCCOCCCc1cccc2c1C(=O)N(C1CCC(=O)NC1=O)C2=O ; NCCOCCOCCCc1cccc2c1C(=O)N(C1CCC(=O)NC1=O)C2=O]

Proceeding analogously as described in Example 10, Step 5 above, but using tert-butyl (2-(2-(3-(2-(2,6-dioxopiperidin-3-yl)-1,3-dioxoisoindolin-4-yl)propoxy)ethoxy)ethyl)carbamate provided the title compound.

### Step 5: tert-butyl (1-(N-(2-(2-(3-(2-(2,6-dioxopiperidin-3-yl)-1,3-dioxoisoindolin-4-yl)propoxy)ethoxy)ethyl)sulfamoyl)piperidin-4-yl)carbamate

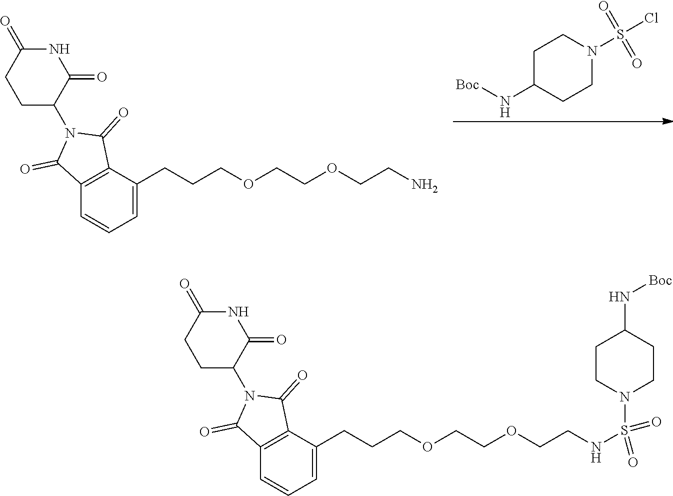 [SMILES: NCCOCCOCCCc1cccc2c1C(=O)N(C1CCC(=O)NC1=O)C2=O ; CC(C)(C)OC(=O)NC1CCN(S(=O)(=O)Cl)CC1 ; CC(C)(C)OC(=O)NC1CCN(S(=O)(=O)NCCOCCOCCCc2cccc3c2C(=O)N(C2CCC(=O)NC2=O)C3=O)CC1]

Proceeding analogously as described in Example 10, Step 6 above, but using 4-(3-(2-(2-aminoethoxy)ethoxy)propyl)-2-(2,6-dioxopiperidin-3-yl)isoindoline-1,3-dione and tert-butyl (1-(chlorosulfonyl)piperidin-4-yl)carbamate provided the title compound.

### Step 6: 4-amino-N-(2-(2-(3-(2-(2,6-dioxopiperidin-3-yl)-1,3-dioxoisoindolin-4-yl)propoxy)ethoxy)ethyl)piperidine-1-sulfonamide

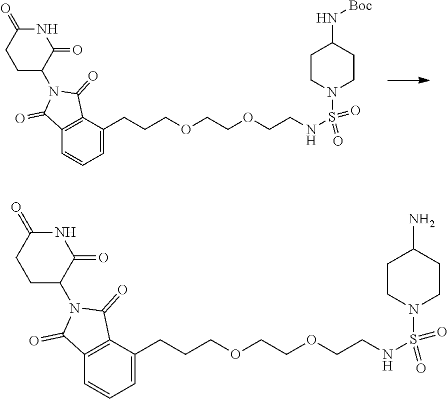 [SMILES: CC(C)(C)OC(=O)NC1CCN(S(=O)(=O)NCCOCCOCCCc2cccc3c2C(=O)N(C2CCC(=O)NC2=O)C3=O)CC1 ; NC1CCN(S(=O)(=O)NCCOCCOCCCc2cccc3c2C(=O)N(C2CCC(=O)NC2=O)C3=O)CC1]

Proceeding analogously as described in Example 10, Step 7 above, but using tert-butyl (1-(N-(2-(2-(3-(2-(2,6-dioxopiperidin-3-yl)-1,3-dioxoisoindolin-4-yl)propoxy)ethoxy)ethyl)sulfamoyl)piperidin-4-yl)carbamate provided the title compound

### Step 7: 4-((8-cyclopentyl-7-oxo-7,8-dihydropyrido[2,3-d]pyrimidin-2-yl)amino)-N-(2-(2-(3-(2-(2,6-dioxopiperidin-3-yl)-1,3-dioxoisoindolin-4-yl)propoxy)ethoxy)ethyl)piperidine-1-sulfonamide

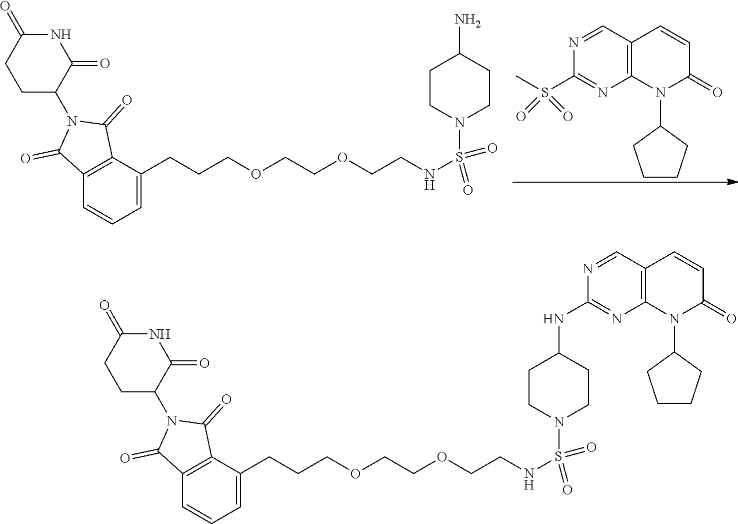

Proceeding analogously as described in Example 10, Step 8 above, but using 4-amino-N-(2-(2-(3-(2-(2,6-dioxopiperidin-3-yl)-1,3-dioxoisoindolin-4-yl)propoxy)ethoxy)ethyl)piperidine-1-sulfonamide and 8-cyclopentyl-2-(methylsulfonyl)-pyrido[2,3-d]pyrimidin-7 (8H)-one provided the title compound. MS (ES, m/z): [M+1]⁺=779.5.

### Example 12

### Synthesis of 5-(3-(3-((4-((8-cyclopentyl-7-oxo-7,8-dihydropyrido[2,3-d]pyrimidin-2-yl)amino)piperidin-1-yl)sulfonyl)phenoxy)azetidin-1-yl)-2-(2,6-dioxopiperidin-3-yl)isoindoline-1,3-dione

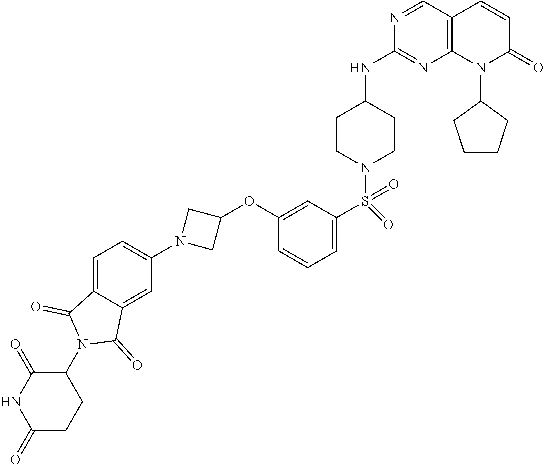 [SMILES: O=C1CCC(N2C(=O)c3ccc(N4CC(Oc5cccc(S(=O)(=O)N6CCC(Nc7ncc8ccc(=O)n(C9CCCC9)c8n7)CC6)c5)C4)cc3C2=O)C(=O)N1]

### Step 1: benzyl (1-((3-methoxyphenyl)sulfonyl)piperidin-4-yl)carbamate

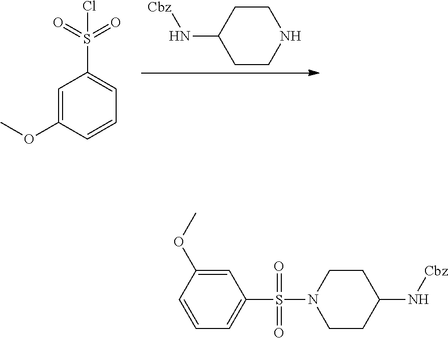 [SMILES: COc1cccc(S(=O)(=O)Cl)c1 ; O=C(NC1CCNCC1)OCc1ccccc1 ; COc1cccc(S(=O)(=O)N2CCC(NC(=O)OCc3ccccc3)CC2)c1]

To a stirred solution of benzyl piperidin-4-ylcarbamate (3.5 g, 14.94 mmol, 1.00 eq.) and TEA (4.52 g, 44.82 mmol, 3.00 eq.) in DCM (50.0 mL) was added a solution of 3-methoxybenzene-1-sulfonyl chloride (3.24 g, 15.68 mmol, 1.05 eq.) in DCM (20.0 mL) dropwise at 0° C. The resulting mixture was stirred at RT for 3 h. The reaction mixture was diluted with DCM and then washed with water. The organic layer was washed with brine, dried over Na₂SO₄, and concentrated. Purification of the crude mixture by silica gel chromatography (EA:PE=1:3) gave the title compound (4.9 g, 81.1%) as a white solid.

### Step 2: 3-((4-aminopiperidin-1-yl)sulfonyl)phenol

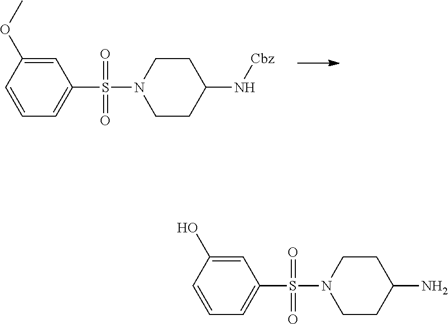 [SMILES: COc1cccc(S(=O)(=O)N2CCC(NC(=O)OCc3ccccc3)CC2)c1 ; NC1CCN(S(=O)(=O)c2cccc(O)c2)CC1]

The solution of benzyl (1-((3-methoxyphenyl)sulfonyl)piperidin-4-yl)carbamate (3.5 g, 8.66 mmol, 1.00 eq.) in CF₃SO₃H (20.0 mL) was stirred under N₂ at 100° C. for 3 h. The reaction mixture was cooled and concentrated to give the title compound (2.2 g, crude) as a brown oil, which was used for next step without further purification.

### Step 3: tert-butyl (1-((3-hydroxyphenyl)(sulfonyl)piperidin-4-yl)carbamate

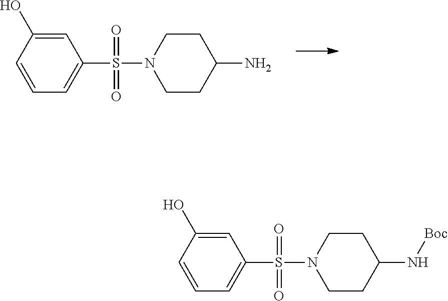 [SMILES: NC1CCN(S(=O)(=O)c2cccc(O)c2)CC1 ; CC(C)(C)OC(=O)NC1CCN(S(=O)(=O)c2cccc(O)c2)CC1]

To a stirred solution of 3-((4-aminopiperidin-1-yl)sulfonyl)phenol (1.0 g, 3.91 mmol, 1.00 eq.) in DCM (20.0 mL) were added TEA (1.18 g, 11.73 mmol, 3.00 eq.) and a solution of (Boc)₂O (852 mg, 3.91 mmol, 1.00 eq.) in DCM (5.0 mL) dropwise at 0° C. The resulting mixture was stirred at RT for 2 h, diluted with DCM and then washed with water. The organic layer was washed with brine, dried over Na₂SO₄, and concentrated. Purification by flash silica gel chromatography (ACN/water=(35%-75%)) to give the title compound (1.07 g, 76.7%) as a white solid.

### Step 4: 1-benzhydrylazetidin-3-yl methanesulfonate

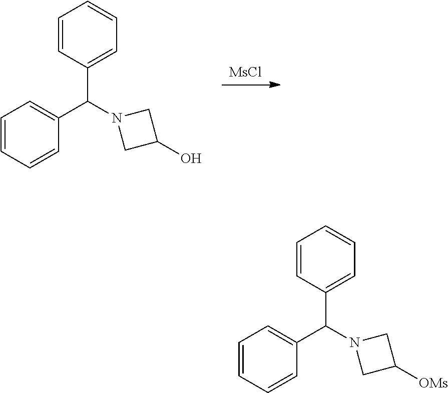 [SMILES: "MsCl" ; OC1CN(C(c2ccccc2)c2ccccc2)C1 ; CS(=O)(=O)OC1CN(C(c2ccccc2)c2ccccc2)C1]

To a stirred solution of 1-benzhydrylazetidin-3-ol (500 mg, 2.09 mmol, 1.00 eq.) in DCM (10.0 mL) was added TEA (633 mg, 6.27 mmol, 3.00 eq.) and MsCl (479 mg, 4.18 mmol, 2.00 eq.) at 0° C. The resulting mixture was stirred at RT overnight, diluted with DCM and then washed with water. The organic layer was washed with brine, dried over Na₂SO₄, concentrated and then purified by silica gel chromatography (EA:PE=1:3) to give the title compound (600 mg, 90.4%) as a white solid.

### Step 5: tert-butyl (1-((3-((1-benzhydrylazetidin-3-yl)oxy)phenyl)sulfonyl)piperidin-4-yl)-carbamate

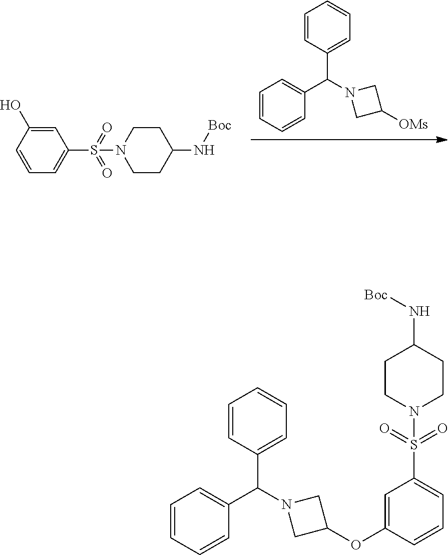 [SMILES: CC(C)(C)OC(=O)NC1CCN(S(=O)(=O)c2cccc(O)c2)CC1 ; CS(=O)(=O)OC1CN(C(c2ccccc2)c2ccccc2)C1 ; CC(C)(C)OC(=O)NC1CCN(S(=O)(=O)c2cccc(OC3CN(C(c4ccccc4)c4ccccc4)C3)c2)CC1]

To a stirred solution of tert-butyl (1-((3-hydroxyphenyl)sulfonyl)piperidin-4-yl)carbamate (533 mg, 1.49 mmol, 1.00 eq.) and 1-benzhydrylazetidin-3-yl methanesulfonate (570 mg, 1.79 mmol, 1.20 eq.) in DMSO (10.0 mL) was added Cs₂CO₃ (1.46 g, 4.49 mmol, 3.00 eq.) at RT. The resulting mixture was stirred at 90° C. under N₂ for 3 h, cooled, diluted with EtOAc and then washed with water. The organic layer was washed with brine, dried over Na₂SO₄, concentrated and then purified by silica gel chromatography (EA:PE=1:3) to give the title compound (523 mg, 60.5%) as a pale yellow solid.

### Step 6: tert-butyl (1-((3-(azetidin-3-yloxy)phenyl)sulfonyl)piperidin-4-yl)carbamate

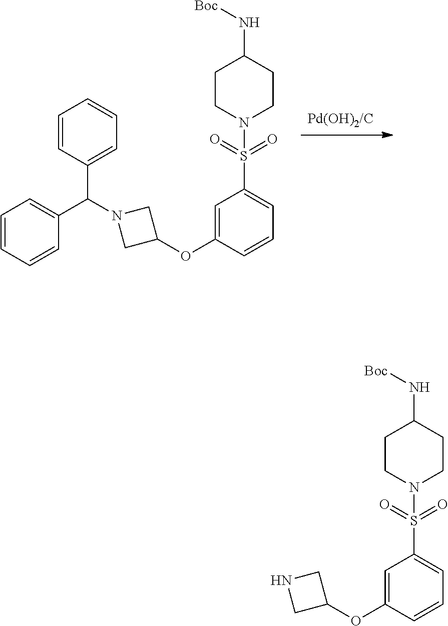 [SMILES: CC(C)(C)OC(=O)NC1CCN(S(=O)(=O)c2cccc(OC3CN(C(c4ccccc4)c4ccccc4)C3)c2)CC1 ; CC(C)(C)OC(=O)NC1CCN(S(=O)(=O)c2cccc(OC3CNC3)c2)CC1]

To a stirred solution of tert-butyl (1-((3-((1-benzhydrylazetidin-3-yl)oxy)phenyl)sulfonyl)-piperidin-4-yl)carbamate (400 mg, 0.69 mmol, 1.00 eq.) in MeOH (15.0 mL) were added Pd(OH)₂/C (20 wt. %, 250 mg) and AcOH (0.5 mL) at RT. The resulting mixture was stirred at 50° C. under H₂ (50 psi) overnight. The reaction mixture was cooled and filtered, and the filtrate was concentrated. The residue was purified by silica gel chromatography (MeOH:DCM=1:15) to give the title compound (230 mg, 81.2%) as a white solid.

### Step 7: tert-butyl (1-((3-((1-(2-(2,6-dioxopiperidin-3-yl)-1,3-dioxoisoindolin-5-yl)azetidin-3-yl)-oxy)phenyl)sulfonyl)piperidin-4-yl)carbamate

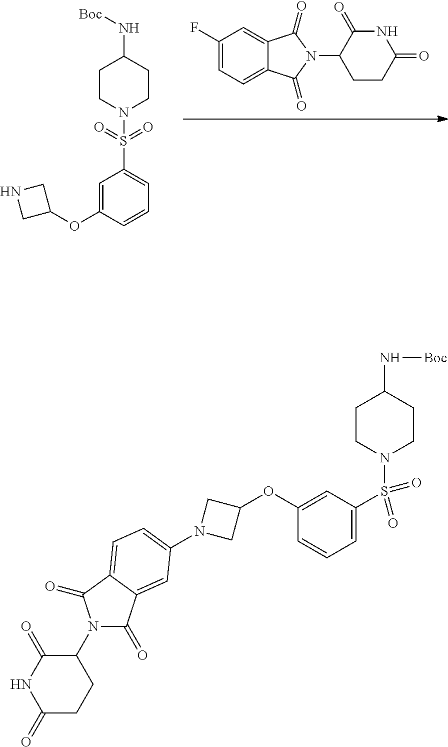 [SMILES: CC(C)(C)OC(=O)NC1CCN(S(=O)(=O)c2cccc(OC3CNC3)c2)CC1 ; O=C1CCC(N2C(=O)c3ccc(F)cc3C2=O)C(=O)N1 ; CC(C)(C)OC(=O)NC1CCN(S(=O)(=O)c2cccc(OC3CN(c4ccc5c(c4)C(=O)N(C4CCC(=O)NC4=O)C5=O)C3)c2)CC1]

Proceeding analogously as described in Example 7, Step 4 above, but using tert-butyl (1-((3-(azetidin-3-yloxy)phenyl)sulfonyl)piperidin-4-yl)carbamate and 2-(2,6-dioxopiperidin-3-yl)-5-fluoroisoindoline-1,3-dione provided the title compound.

### Step 8: 5-(3-(3-((4-aminopiperidin-1-yl)sulfonyl)phenoxy)azetidin-1-yl)-2-(2,6-dioxopiperidin-3-yl)isoindoline-1,3-dione

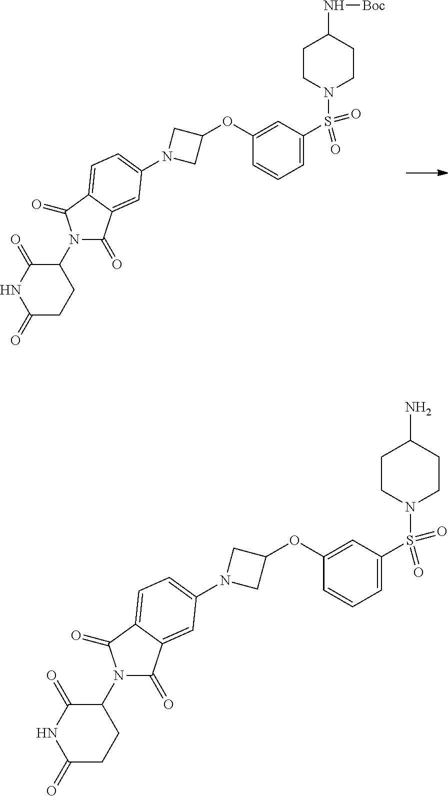 [SMILES: CC(C)(C)OC(=O)NC1CCN(S(=O)(=O)c2cccc(OC3CN(c4ccc5c(c4)C(=O)N(C4CCC(=O)NC4=O)C5=O)C3)c2)CC1 ; NC1CCN(S(=O)(=O)c2cccc(OC3CN(c4ccc5c(c4)C(=O)N(C4CCC(=O)NC4=O)C5=O)C3)c2)CC1]

Proceeding analogously as described in Example 7, Step 5 above, but using tert-butyl (1-((3-((1-(2-(2,6-dioxopiperidin-3-yl)-1,3-dioxoisoindolin-5-yl)azetidin-3-yl)oxy)phenyl)-sulfonyl)piperidin-4-yl)carbamate provided the title compound.

### Step 9: 5-(3-(3-((4-((8-cyclopentyl-7-oxo-7,8-dihydropyrido[2,3-d]pyrimidin-2-yl)amino)-piperidin-1-yl)sulfonyl)phenoxy)azetidin-1-yl)-2-(2,6-dioxopiperidin-3-yl)isoindoline-1,3-dione

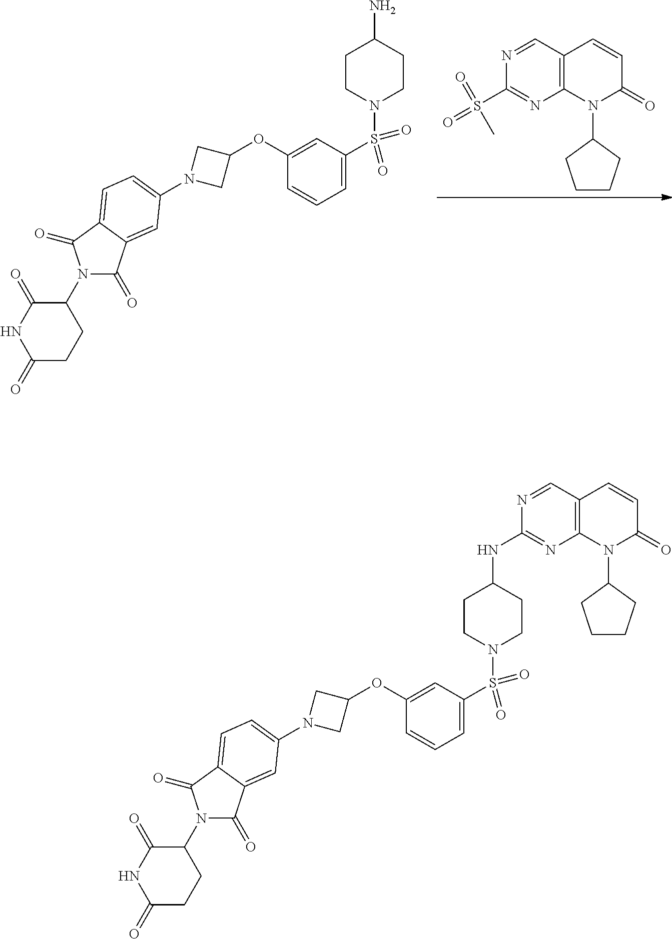

Proceeding analogously as described in Example 7, Step 6 above, but using 5-(3-(3-((4-aminopiperidin-1-yl)sulfonyl)phenoxy)azetidin-1-yl)-2-(2,6-dioxopiperidin-3-yl)isoindoline-1,3-dione and 8-cyclopentyl-2-(methylsulfonyl)pyrido[2,3-d]pyrimidin-7 (8H)-one provided the title compound. MS (ES, m/z): [M+1]⁺=781.4.

### Example 13

### Synthesis of 3-(4-(3-((1-((1-((4-((8-cyclopentyl-7-oxo-7,8-dihydropyrido[2,3-d]pyrimidin-2-yl)amino)piperidin-1-yl)sulfonyl)piperidin-4-yl)methyl)piperidin-4-yl)oxy)prop-1-yn-1-yl)-3-methyl-2-oxo-2,3-dihydro-1H-benzo[d]imidazol-1-yl)piperidine-2,6-dione

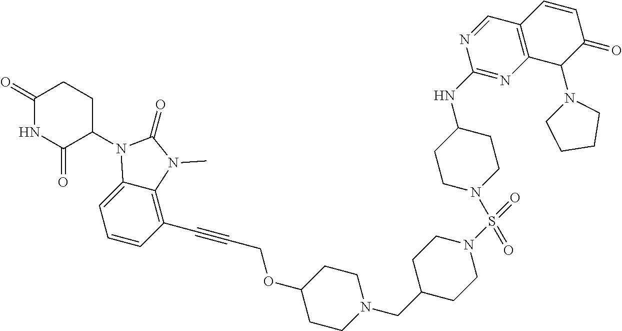 [SMILES: Cn1c(=O)n(C2CCC(=O)NC2=O)c2cccc(C#CCOC3CCN(CC4CCN(S(=O)(=O)N5CCC(Nc6ncc7c(n6)C(N6CCCC6)C(=O)C=C7)CC5)CC4)CC3)c21]

### Step 1: tert-butyl 4-((4-((3-(1-(2,6-dioxopiperidin-3-yl)-3-methyl-2-oxo-2,3-dihydro-1H-benzo[d]imidazol-4-yl)prop-2-yn-1-yl)oxy)piperidin-1-yl)methyl)piperidine-1-carboxylate

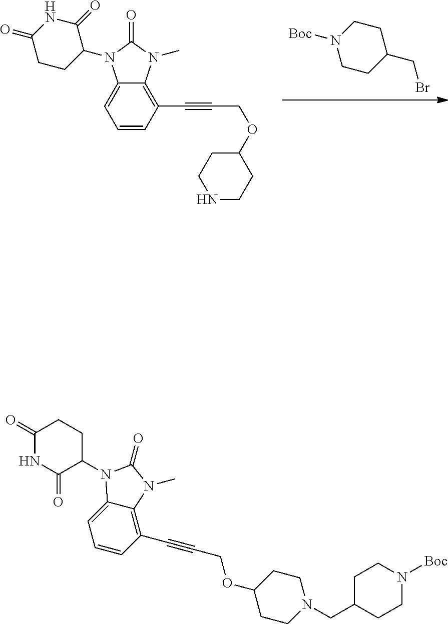 [SMILES: Cn1c(=O)n(C2CCC(=O)NC2=O)c2cccc(C#CCOC3CCNCC3)c21 ; CC(C)(C)OC(=O)N1CCC(CBr)CC1 ; Cn1c(=O)n(C2CCC(=O)NC2=O)c2cccc(C#CCOC3CCN(CC4CCN(C(=O)OC(C)(C)C)CC4)CC3)c21]

A mixture of 3-(3-methyl-2-oxo-4-(3-(piperidin-4-yloxy)prop-1-yn-1-yl)-2,3-dihydro-1H-benzo[d]imidazol-1-yl)piperidine-2,6-dione (300 mg, 0.76 mmol, 1.00 eq., prepared by proceeding as described in Example 11, Steps 1 and 2 above), tert-butyl 4-(bromomethyl)piperidine-1-carboxylate (421 mg, 1.51 mmol, 2.00 eq.), NaI (114 mg, 0.76 mmol, 1.00 eq.), K₂CO₃ (634.8 mg, 4.59 mmol, 6.00 eq.) in ACN (5.0 mL) was stirred at 70° C. overnight. The reaction mixture was cooled, concentrated and then purified with chromatograph on silica gel (DCM/MeOH=20/1) to give the title compound (150 mg, 32.9%) as a yellow solid.

### Step 2: 3-(3-methyl-2-oxo-4-(3-((1-(piperidin-4-ylmethyl)piperidin-4-yl)oxy)prop-1-yn-1-yl)-2,3-dihydro-1H-benzo[d]imidazol-1-yl)piperidine-2,6-dione

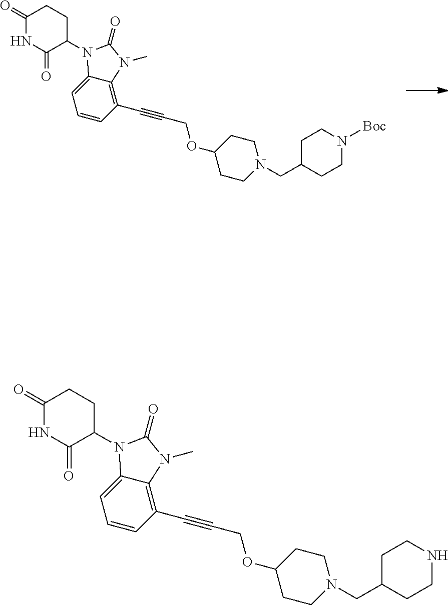 [SMILES: Cn1c(=O)n(C2CCC(=O)NC2=O)c2cccc(C#CCOC3CCN(CC4CCN(C(=O)OC(C)(C)C)CC4)CC3)c21 ; Cn1c(=O)n(C2CCC(=O)NC2=O)c2cccc(C#CCOC3CCN(CC4CCNCC4)CC3)c21]

Proceeding analogously as described in Example 10, Step 5 above, but using tert-butyl 4-((4-((3-(1-(2,6-dioxopiperidin-3-yl)-3-methyl-2-oxo-2,3-dihydro-1H-benzo[d]imidazol-4-yl)prop-2-yn-1-yl)oxy)piperidin-1-yl)methyl)piperidine-1-carboxylate provided the title compound.

### Step 3: tert-butyl (1-((4-((4-((3-(1-(2,6-dioxopiperidin-3-yl)-3-methyl-2-oxo-2,3-dihydro-1H-benzo[d]imidazol-4-yl)prop-2-yn-1-yl)oxy)piperidin-1-yl)methyl)piperidin-1-yl)sulfonyl)piperidin-4-yl)carbamate

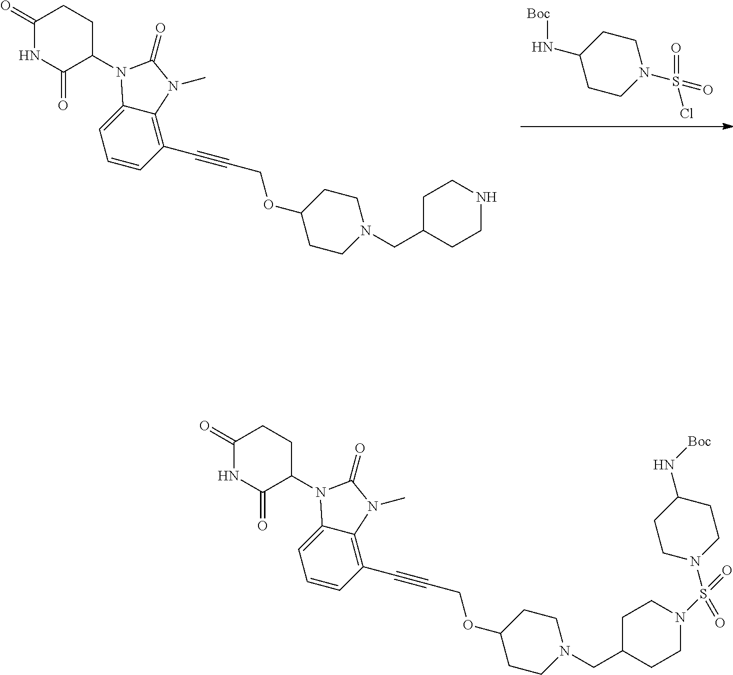 [SMILES: Cn1c(=O)n(C2CCC(=O)NC2=O)c2cccc(C#CCOC3CCN(CC4CCNCC4)CC3)c21 ; CC(C)(C)OC(=O)NC1CCN(S(=O)(=O)Cl)CC1 ; Cn1c(=O)n(C2CCC(=O)NC2=O)c2cccc(C#CCOC3CCN(CC4CCN(S(=O)(=O)N5CCC(NC(=O)OC(C)(C)C)CC5)CC4)CC3)c21]

Proceeding analogously as described in Example 10, Step 6 above, but using 3-(3-methyl-2-oxo-4-(3-((1-(piperidin-4-ylmethyl)piperidin-4-yl)oxy)prop-1-yn-1-yl)-2,3-dihydro-1H-benzo[d]imidazol-1-yl)piperidine-2,6-dione and tert-butyl(1-(chlorosulfonyl)piperidin-4-yl)carbamate provided the title compound.

### Step 4: 3-(4-(3-((1-((1-((4-aminopiperidin-1-yl)sulfonyl)piperidin-4-yl)methyl)piperidin-4-yl)-oxy)prop-1-yn-1-yl)-3-methyl-2-oxo-2,3-dihydro-1H-benzo[d]imidazol-1-yl)piperidine-2,6-dione

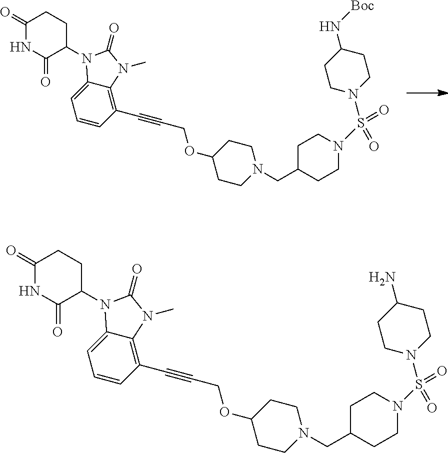 [SMILES: Cn1c(=O)n(C2CCC(=O)NC2=O)c2cccc(C#CCOC3CCN(CC4CCN(S(=O)(=O)N5CCC(NC(=O)OC(C)(C)C)CC5)CC4)CC3)c21 ; Cn1c(=O)n(C2CCC(=O)NC2=O)c2cccc(C#CCOC3CCN(CC4CCN(S(=O)(=O)N5CCC(N)CC5)CC4)CC3)c21]

Proceeding analogously as described in Example 10, Step 7 above, but using tert-butyl (1-((4-((4-((3-(1-(2,6-dioxopiperidin-3-yl)-3-methyl-2-oxo-2,3-dihydro-1H-benzo[d]imidazol-4-yl)-prop-2-yn-1-yl)oxy)piperidin-1-yl)methyl)piperidin-1-yl)sulfonyl)piperidin-4-yl)carbamate provided the title compound.

### Step 5: 3-(4-(3-((1-((1-((4-((8-cyclopentyl-7-oxo-7,8-dihydropyrido[2,3-d]pyrimidin-2-yl)amino)-piperidin-1-yl)sulfonyl)piperidin-4-yl)methyl)piperidin-4-yl)oxy)prop-1-yn-1-yl)-3-methyl-2-oxo-2,3-dihydro-1H-benzo[d]imidazol-1-yl)piperidine-2,6-dione

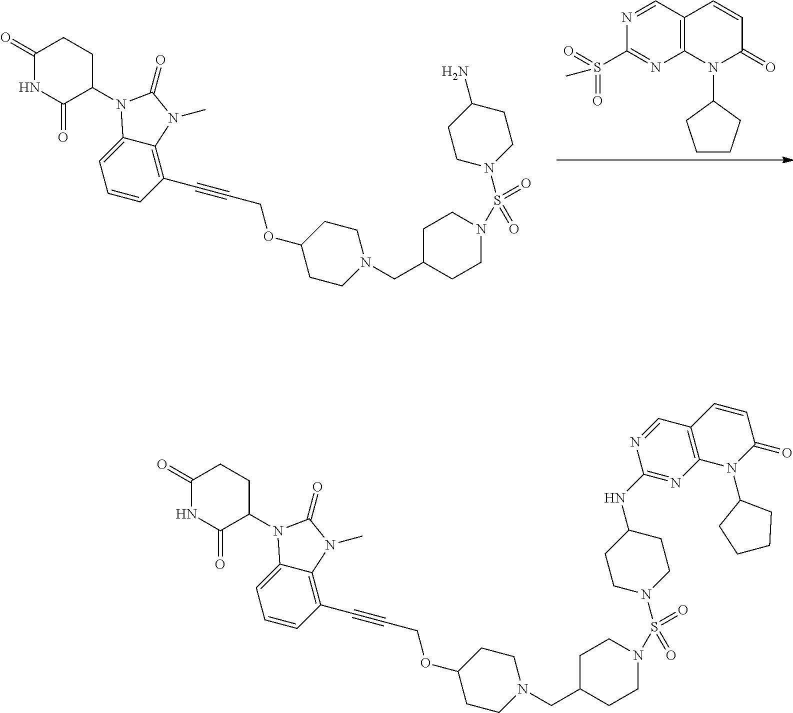 [SMILES: Cn1c(=O)n(C2CCC(=O)NC2=O)c2cccc(C#CCOC3CCN(CC4CCN(S(=O)(=O)N5CCC(N)CC5)CC4)CC3)c21 ; CS(=O)(=O)c1ncc2ccc(=O)n(C3CCCC3)c2n1 ; Cn1c(=O)n(C2CCC(=O)NC2=O)c2cccc(C#CCOC3CCN(CC4CCN(S(=O)(=O)N5CCC(Nc6ncc7ccc(=O)n(C8CCCC8)c7n6)CC5)CC4)CC3)c21]

Proceeding analogously as described in Example 10, Step 8 above, but using 3-(4-(3-((1-((1-((4-aminopiperidin-1-yl)sulfonyl)piperidin-4-yl)methyl)piperidin-4-yl)oxy)prop-1-yn-1-yl)-3-methyl-2-oxo-2,3-dihydro-1H-benzo[d]imidazol-1-yl)piperidine-2,6-dione and 8-cyclopentyl-2-(methylsulfonyl)pyrido[2,3-d]pyrimidin-7 (8H)-one provided the title compound. MS (ES, m/z): [M+1]⁺=869.6.

### Example 14

### Synthesis of 3-(4-(3-((1-((4-((8-cyclopentyl-7-oxo-7,8-dihydropyrido[2,3-d]pyrimidin-2-yl)amino)piperidin-1-yl)sulfonyl)piperidin-4-yl)oxy)prop-1-yn-1-yl)-3-methyl-2-oxo-2,3-dihydro-1H-benzo[d]imidazol-1-yl)piperidine-2,6-dione

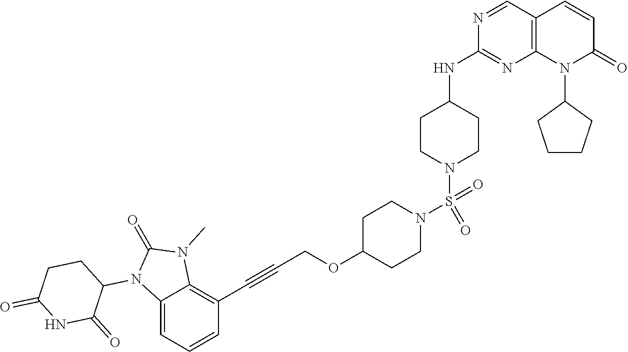 [SMILES: Cn1c(=O)n(C2CCC(=O)NC2=O)c2cccc(C#CCOC3CCN(S(=O)(=O)N4CCC(Nc5ncc6ccc(=O)n(C7CCCC7)c6n5)CC4)CC3)c21]

### Step 1: 3-hydroxy-1-(4-methoxybenzyl)piperidine-2,6-dione

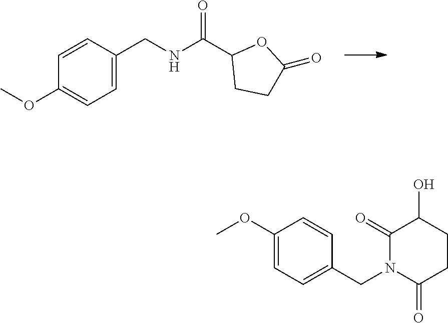 [SMILES: COc1ccc(CNC(=O)C2CCC(=O)O2)cc1 ; COc1ccc(CN2C(=O)CCC(O)C2=O)cc1]

To a stirred mixture of N-(4-methoxybenzyl)-5-oxotetrahydrofuran-2-carboxamide (5.0 g, 20.06 mmol, 1.00 eq.) in THF (50.0 mL) was added t-BuOK (2.3 g, 20.50 mmol, 1.02 eq.) at −78° C. After stirring at −78° C. for 1 h, the reaction mixture was quenched with saturated aqueous NH₄Cl and then extracted with EtOAc. The organic layer was washed with brine, dried over Na₂SO₄, concentrated and then purified with silica gel chromatograph (PE/EA=3/1) to give the title compound (3.0 g, 60.0%) as a white solid.

### Step 2: 1-(4-methoxybenzyl)-2,6-dioxopiperidin-3-yl trifluoromethanesulfonate

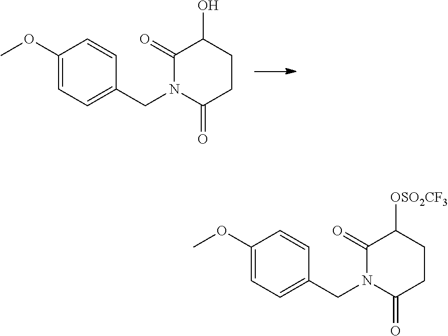

To a stirred solution of 3-hydroxy-1-(4-methoxybenzyl)piperidine-2,6-dione (1.9 g, 7.62 mmol, 1.00 eq.) and pyridine (1.2 g, 15.17 mmol, 1.99 eq.) in DCM (40.0 mL) was added trifluoromethanesulfonic anhydride (3.2 g, 11.34 mmol, 1.49 eq.) slowly at 0° C. After stirring at 0° C. for 2 h, the reaction mixture was quenched with water and then extracted with DCM. The organic layer was washed with brine, dried over Na₂SO₄, concentrated and then purified by silica gel chromatograph (PE/EA=5/1) to give the title compound (1.3 g, 44.8%) as a yellow oil.

### Step 3: 3-(4-bromo-3-methyl-2-oxo-2,3-dihydro-1H-benzo[d]imidazol-1-yl)-1-(4-methoxy-benzyl)piperidine-2,6-dione

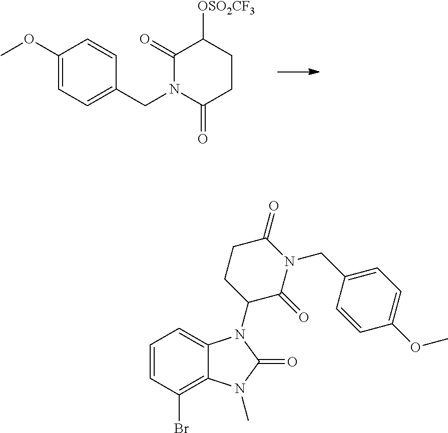

To a stirred solution of 7-bromo-1-methyl-1H-benzo[d]imidazol-2 (3H)-one (1.1 g, 4.84 mmol, 1.23 eq.) in THF (30.0 mL) was added t-BuOK (632 mg, 5.63 mmol, 1.43 eq.) at 0° C. After stirring at 0° C. for 0.5 h, a solution of 1-(4-methoxybenzyl)-2,6-dioxopiperidin-3-yl trifluoromethanesulfonate (1.5 g, 3.93 mmol, 1.00 eq.) in THF (10.0 mL) was added at 0° C. The reaction mixture was stirred at 0° C. continually for 1 h, diluted with water and then extracted with EtOAc. The organic layer was washed with brine, dried over Na₂SO₄, concentrated and then purified by silica gel chromatograph (PE/EA=2/1) to give the title compound (1.2 g, 66.7%) as a white solid.

### Step 4: 3-(4-bromo-3-methyl-2-oxo-2,3-dihydro-1H-benzo[d]imidazol-1-yl)piperidine-2,6-dione

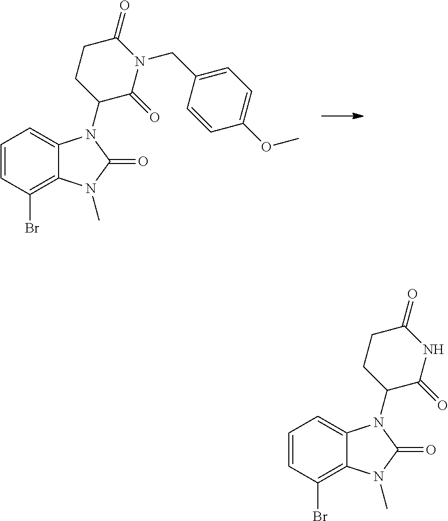 [SMILES: COc1ccc(CN2C(=O)CCC(n3c(=O)n(C)c4c(Br)cccc43)C2=O)cc1 ; Cn1c(=O)n(C2CCC(=O)NC2=O)c2cccc(Br)c21]

A mixture of 3-(4-bromo-3-methyl-2-oxo-2,3-dihydro-1H-benzo[d]imidazol-1-yl)-1-(4-methoxybenzyl)piperidine-2,6-dione (900 mg, 1.96 mmol, 1.00 eq.) in toluene/methanesulfonic acid=2/1 (3.0 mL) was stirred at 120° C. for 3 h. The reaction mixture was cooled, concentrated and poured into ice water. The resulting mixture was filtered, and the cake was dried to give the title compound (400 mg, 60.2%) as a white solid.

### Step 5: tert-butyl 4-(prop-2-yn-1-yloxy)piperidine-1-carboxylate

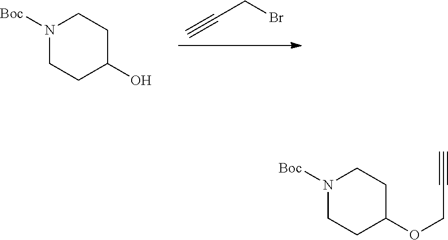

To a stirred solution of tert-butyl 4-hydroxypiperidine-1-carboxylate (1.0 g, 4.97 mmol, 1.00 eq.) in THF (20.0 mL) was added NaH (60%, 240 mg, 6.00 mmol, 1.21 eq.) at 0° C., followed by 3-bromoprop-1-yne (704 mg, 5.92 mmol 1.19 eq.). The resulting mixture was stirred at RT for 2 h, quenched with water and then extracted with EtOAc. The organic layer was washed with brine, dried over Na₂SO₄, concentrated and then purified by silica gel chromatograph (PE/EA=10/1) to give the title compound (1.0 g, 84.1%) as a white solid.

### Step 6: tert-butyl 4-((3-(1-(2,6-dioxopiperidin-3-yl)-3-methyl-2-oxo-2,3-dihydro-1H-benzo[d]imidazol-4-yl)prop-2-yn-1-yl)oxy)piperidine-1-carboxylate

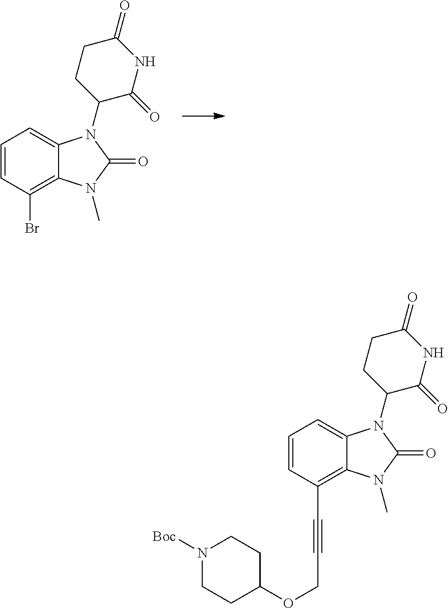 [SMILES: Cn1c(=O)n(C2CCC(=O)NC2=O)c2cccc(Br)c21 ; Cn1c(=O)n(C2CCC(=O)NC2=O)c2cccc(C#CCOC3CCN(C(=O)OC(C)(C)C)CC3)c21]

Proceeding analogously as described in Example 10, Step 3 above, but using 3-(4-bromo-3-methyl-2-oxo-2,3-dihydro-1H-benzo[d]imidazol-1-yl)piperidine-2,6-dione and tert-butyl 4-(prop-2-yn-1-yloxy)piperidine-1-carboxylate in DMF provided the title compound.

### Step 6: 3-(3-methyl-2-oxo-4-(3-(piperidin-4-yloxy)prop-1-yn-1-yl)-2,3-dihydro-1H-benzo[d]imidazol-1-yl)piperidine-2,6-dione

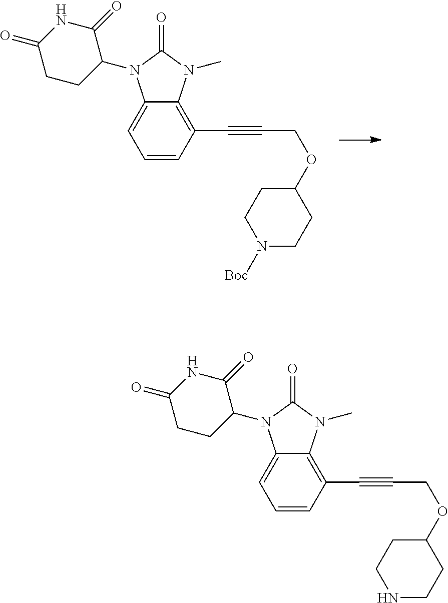 [SMILES: Cn1c(=O)n(C2CCC(=O)NC2=O)c2cccc(C#CCOC3CCN(C(=O)OC(C)(C)C)CC3)c21 ; Cn1c(=O)n(C2CCC(=O)NC2=O)c2cccc(C#CCOC3CCNCC3)c21]

Proceeding analogously as described in Example 10, Step 5 above, but using tert-butyl 4-((3-(1-(2,6-dioxopiperidin-3-yl)-3-methyl-2-oxo-2,3-dihydro-1H-benzo[d]imidazol-4-yl)prop-2-yn-1-yl)oxy)piperidine-1-carboxylate provided the title compound.

### Step 7: tert-butyl (1-((4-((3-(1-(2,6-dioxopiperidin-3-yl)-3-methyl-2-oxo-2,3-dihydro-1H-benzo[d]imidazol-4-yl)prop-2-yn-1-yl)oxy)piperidin-1-yl)sulfonyl)piperidin-4-yl)carbamate

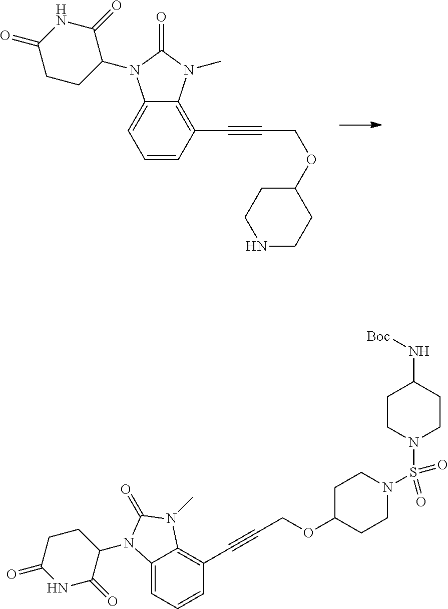 [SMILES: Cn1c(=O)n(C2CCC(=O)NC2=O)c2cccc(C#CCOC3CCNCC3)c21 ; Cn1c(=O)n(C2CCC(=O)NC2=O)c2cccc(C#CCOC3CCN(S(=O)(=O)N4CCC(NC(=O)OC(C)(C)C)CC4)CC3)c21]

Proceeding analogously as described in Example 10, Step 6 above, but using 3-(3-methyl-2-oxo-4-(3-(piperidin-4-yloxy)prop-1-yn-1-yl)-2,3-dihydro-1H-benzo[d]imidazol-1-yl)piperidine-2,6-dione and tert-butyl (1-(chlorosulfonyl)piperidin-4-yl)carbamate in DMF provided the title compound.

### Step 8: 3-(4-(3-((1-((4-aminopiperidin-1-yl)sulfonyl)piperidin-4-yl)oxy)prop-1-yn-1-yl)-3-methyl-2-oxo-2,3-dihydro-1H-benzo[d]imidazol-1-yl)piperidine-2,6-dione

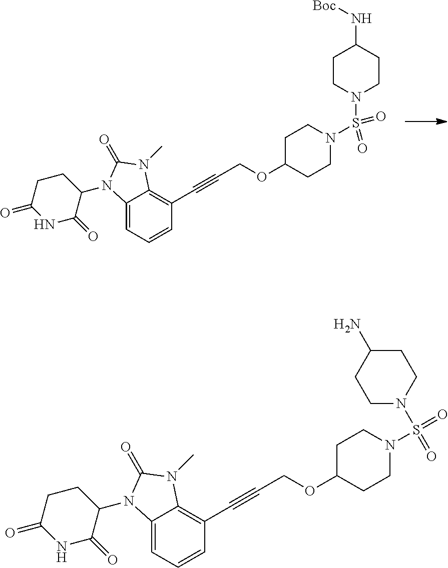 [SMILES: Cn1c(=O)n(C2CCC(=O)NC2=O)c2cccc(C#CCOC3CCN(S(=O)(=O)N4CCC(NC(=O)OC(C)(C)C)CC4)CC3)c21 ; Cn1c(=O)n(C2CCC(=O)NC2=O)c2cccc(C#CCOC3CCN(S(=O)(=O)N4CCC(N)CC4)CC3)c21]

Proceeding analogously as described in Example 10, Step 7 above, but using tert-butyl (1-((4-((3-(1-(2,6-dioxopiperidin-3-yl)-3-methyl-2-oxo-2,3-dihydro-1H-benzo[d]imidazol-4-yl)prop-2-yn-1-yl)oxy)piperidin-1-yl)sulfonyl)piperidin-4-yl)carbamate provided the title compound.

### Step 9: 3-(4-(3-((1-((4-((8-cyclopentyl-7-oxo-7,8-dihydropyrido[2,3-d]pyrimidin-2-yl)amino)piperidin-1-yl)sulfonyl)piperidin-4-yl)oxy)prop-1-yn-1-yl)-3-methyl-2-oxo-2,3-dihydro-1H-benzo[d]imidazol-1-yl)piperidine-2,6-dione

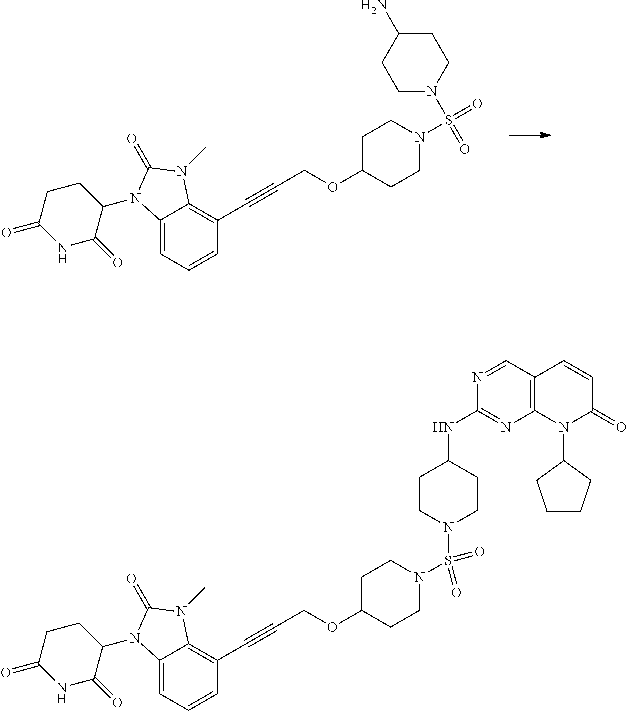 [SMILES: Cn1c(=O)n(C2CCC(=O)NC2=O)c2cccc(C#CCOC3CCN(S(=O)(=O)N4CCC(N)CC4)CC3)c21 ; Cn1c(=O)n(C2CCC(=O)NC2=O)c2cccc(C#CCOC3CCN(S(=O)(=O)N4CCC(Nc5ncc6ccc(=O)n(C7CCCC7)c6n5)CC4)CC3)c21]

Proceeding analogously as described in Example 10, Step 8 above, but using 3-(4-(3-((1-((4-aminopiperidin-1-yl)sulfonyl)piperidin-4-yl)oxy)prop-1-yn-1-yl)-3-methyl-2-oxo-2,3-dihydro-1H-benzo[d]imidazol-1-yl)piperidine-2,6-dione and 8-cyclopentyl-2-(methylsulfonyl)pyrido[2,3-d]pyrimidin-7 (8H)-one in DMSO provided the title compound. MS (ES, m/z): [M+1]⁺=772.4.

### Example 15

### Synthesis of 4-((8-cyclopentyl-7-oxo-7,8-dihydropyrido[2,3-d]pyrimidin-2-yl)amino)-N-(4-((2-(2,6-dioxopiperidin-3-yl)-1,3-dioxoisoindolin-4-yl)amino)cyclohexyl)-N-methylpiperidine-1-sulfonamide

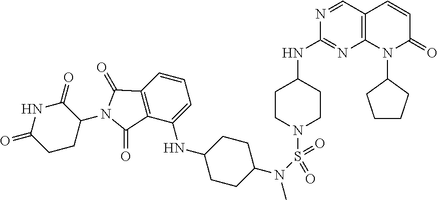 [SMILES: CN(C1CCC(Nc2cccc3c2C(=O)N(C2CCC(=O)NC2=O)C3=O)CC1)S(=O)(=O)N1CCC(Nc2ncc3ccc(=O)n(C4CCCC4)c3n2)CC1]

### Step 1: 4-aminocyclohexanone hydrochloride

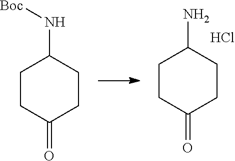 [SMILES: CC(C)(C)OC(=O)NC1CCC(=O)CC1 ; NC1CCC(=O)CC1 ; Cl]

A mixture of tert-butyl (4-oxocyclohexyl)carbamate (500 mg, 2.34 mmol, 1.00 eq.) in a solution of HCl in ethyl acetate (1.0 M, 10.0 mL) was stirred at RT for 1 h. The reaction mixture was concentrated to give the title compound (500 mg, crude), which was used for next step without further purification.

### Step 2: 2-(2,6-dioxopiperidin-3-yl)-4-((4-oxocyclohexyl)amino)isoindoline-1,3-dione

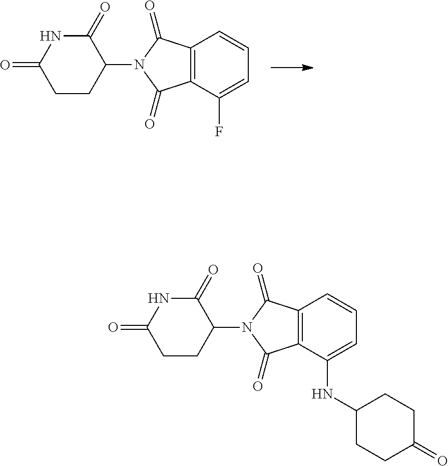 [SMILES: O=C1CCC(N2C(=O)c3cccc(F)c3C2=O)C(=O)N1 ; O=C1CCC(Nc2cccc3c2C(=O)N(C2CCC(=O)NC2=O)C3=O)CC1]

A mixture of 2-(2,6-dioxopiperidin-3-yl)-4-fluoroisoindoline-1,3-dione (276 mg, 1.00 mmol, 1.00 eq.) and 4-aminocyclohexanone hydrochloride (300 mg, 2.00 mmol, 2.00 eq.) in NMP (2.5 mL) was stirred at 140° C. under microwave for 3 h. The reaction mixture was cooled, diluted with DCM and then washed with brine. The organic layer was concentrated, and then the residue was triturated with DCM, filtered to give the title compound (160 mg, 43.3%) as a yellow solid.

### Step 3: 2-(2,6-dioxopiperidin-3-yl)-4-((4-(methylamino)cyclohexyl)amino)isoindoline-1,3-dione

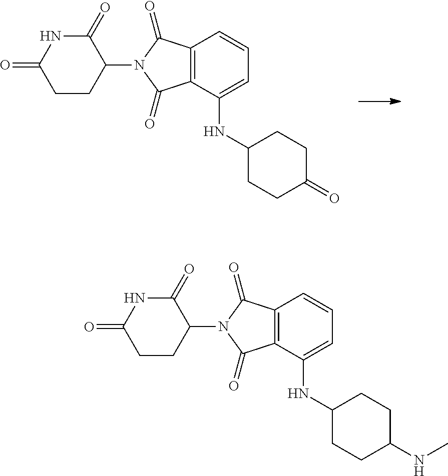 [SMILES: O=C1CCC(Nc2cccc3c2C(=O)N(C2CCC(=O)NC2=O)C3=O)CC1 ; CNC1CCC(Nc2cccc3c2C(=O)N(C2CCC(=O)NC2=O)C3=O)CC1]

To a stirred mixture of 2-(2,6-dioxopiperidin-3-yl)-4-((4-oxocyclohexyl)amino)-isoindoline-1,3-dione (200 mg, 0.54 mmol, 1.00 eq.) and methylamine (40% in MeOH, 210 mg, 2.71 mmol, 5.02 eq.) in MeOH/DCE (2.0 mL/2.0 mL) was added one drop of AcOH. The resulting mixture was stirred at RT for 1 h, and then NaBH(OAc)₃ (345 mg, 1.63 mmol, 3.02 eq.) was added. The reaction mixture was stirred at RT overnight, diluted with DCM, washed with saturated aqueous NaHCO₃ and then brine. The organic layer was dried over Na₂SO₄ and then concentrated to give the title compound (110 mg, 53.7%) as a yellow solid.

### Step 4: tert-butyl (1-(N-(4-((2-(2,6-dioxopiperidin-3-yl)-1,3-dioxoisoindolin-4-yl)amino)-cyclohexyl)-N-methylsulfamoyl)piperidin-4-yl)carbamate

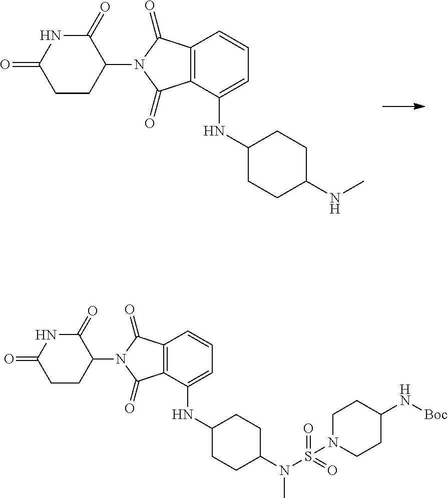 [SMILES: CNC1CCC(Nc2cccc3c2C(=O)N(C2CCC(=O)NC2=O)C3=O)CC1 ; CN(C1CCC(Nc2cccc3c2C(=O)N(C2CCC(=O)NC2=O)C3=O)CC1)S(=O)(=O)N1CCC(NC(=O)OC(C)(C)C)CC1]

Proceeding analogously as described in Example 10, Step 6 above, but using 2-(2,6-dioxopiperidin-3-yl)-4-((4-(methylamino)cyclohexyl)amino)isoindoline-1,3-dione and tert-butyl (1-(chlorosulfonyl)piperidin-4-yl)carbamate provided the title compound.

### Step 5: 4-amino-N-(4-((2-(2,6-dioxopiperidin-3-yl)-1,3-dioxoisoindolin-4-yl)amino)cyclohexyl)-N-methylpiperidine-1-sulfonamide 2,2,2-trifluoroacetate

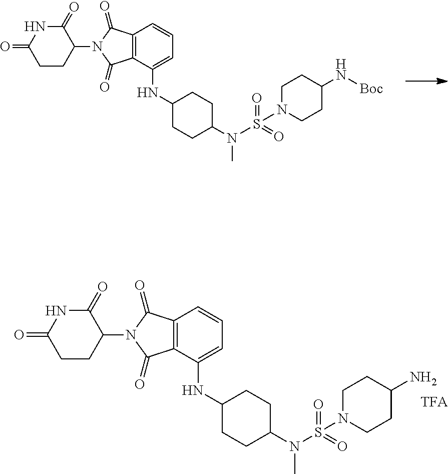 [SMILES: CN(C1CCC(Nc2cccc3c2C(=O)N(C2CCC(=O)NC2=O)C3=O)CC1)S(=O)(=O)N1CCC(NC(=O)OC(C)(C)C)CC1 ; CN(C1CCC(Nc2cccc3c2C(=O)N(C2CCC(=O)NC2=O)C3=O)CC1)S(=O)(=O)N1CCC(N)CC1]

Proceeding analogously as described in Example 10, Step 7 above, but using tert-butyl (1-(N-(4-((2-(2,6-dioxopiperidin-3-yl)-1,3-dioxoisoindolin-4-yl)amino)cyclohexyl)-N-methylsulfamoyl)piperidin-4-yl)carbamate provided the title compound.

### Step 6: 4-((8-cyclopentyl-7-oxo-7,8-dihydropyrido[2,3-d]pyrimidin-2-yl)amino)-N-(4-((2-(2,6-dioxopiperidin-3-yl)-1,3-dioxoisoindolin-4-yl)amino)cyclohexyl)-N-methylpiperidine-1-sulfonamide

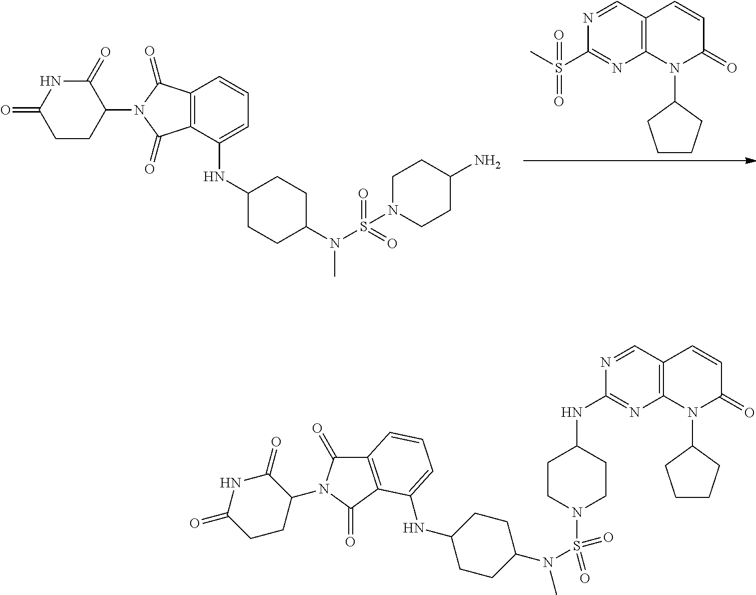 [SMILES: CN(C1CCC(Nc2cccc3c2C(=O)N(C2CCC(=O)NC2=O)C3=O)CC1)S(=O)(=O)N1CCC(N)CC1 ; CS(=O)(=O)c1ncc2ccc(=O)n(C3CCCC3)c2n1 ; CN(C1CCC(Nc2cccc3c2C(=O)N(C2CCC(=O)NC2=O)C3=O)CC1)S(=O)(=O)N1CCC(Nc2ncc3ccc(=O)n(C4CCCC4)c3n2)CC1]

Proceeding analogously as described in Example 10, Step 8 above, but using 4-amino-N-(4-((2-(2,6-dioxopiperidin-3-yl)-1,3-dioxoisoindolin-4-yl)amino)cyclohexyl)-N-methylpiperidine-1-sulfonamide 2,2,2-trifluoroacetate and 8-cyclopentyl-2-(methylsulfonyl)pyrido[2,3-d]pyrimidin-7 (8H)-one provided the title compound. MS (ES, m/z): [M+1]⁺=760.3.

### Example 16

### Synthesis of 3-(4-(3-((1-((4-((8-cyclopentyl-7-oxo-7,8-dihydropyrido[2,3-d]pyrimidin-2-yl)amino)piperidin-1-yl)sulfonyl)piperidin-4-yl)oxy)propyl)-3-methyl-2-oxo-2,3-dihydro-1H-benzo[d]imidazol-1-yl)piperidine-2,6-dione

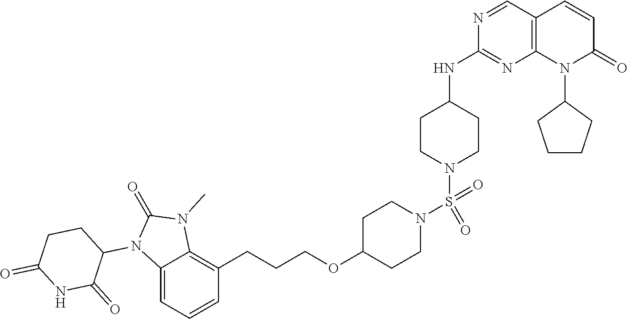 [SMILES: Cn1c(=O)n(C2CCC(=O)NC2=O)c2cccc(CCCOC3CCN(S(=O)(=O)N4CCC(Nc5ncc6ccc(=O)n(C7CCCC7)c6n5)CC4)CC3)c21]

### Step 1: tert-butyl 4-((3-(1-(2,6-dioxopiperidin-3-yl)-3-methyl-2-oxo-2,3-dihydro-1H-benzo[d]-imidazol-4-yl)prop-2-yn-1-yl)oxy)piperidine-1-carboxylate

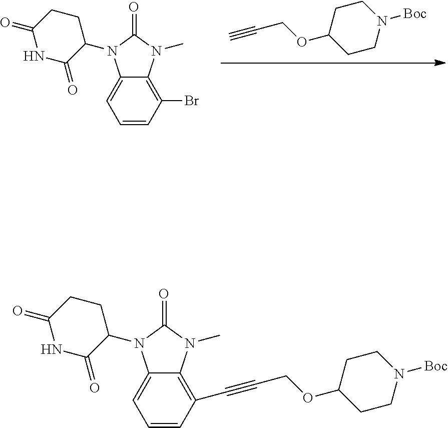 [SMILES: Cn1c(=O)n(C2CCC(=O)NC2=O)c2cccc(Br)c21 ; C#CCOC1CCN(C(=O)OC(C)(C)C)CC1 ; Cn1c(=O)n(C2CCC(=O)NC2=O)c2cccc(C#CCOC3CCN(C(=O)OC(C)(C)C)CC3)c21]

Proceeding analogously as described in Example 10, Step 3 above, but using 3-(4-bromo-3-methyl-2-oxo-2,3-dihydro-1H-benzo[d]imidazol-1-yl)piperidine-2,6-dione and tert-butyl 4-(prop-2-yn-1-yloxy)piperidine-1-carboxylate provided the title compound.

### Step 2: 3-(4-(3-((1-((4-((8-cyclopentyl-7-oxo-7,8-dihydropyrido[2,3-d]pyrimidin-2-yl)amino)-piperidin-1-yl)sulfonyl)piperidin-4-yl)oxy)propyl)-3-methyl-2-oxo-2,3-dihydro-1H-benzo[d]imidazol-1-yl)piperidine-2,6-dione

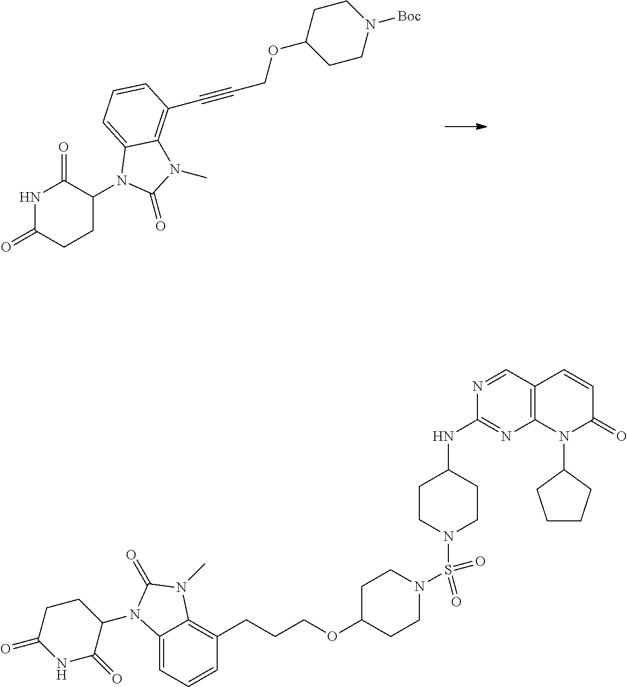 [SMILES: Cn1c(=O)n(C2CCC(=O)NC2=O)c2cccc(C#CCOC3CCN(C(=O)OC(C)(C)C)CC3)c21 ; Cn1c(=O)n(C2CCC(=O)NC2=O)c2cccc(CCCOC3CCN(S(=O)(=O)N4CCC(Nc5ncc6ccc(=O)n(C7CCCC7)c6n5)CC4)CC3)c21]

tert-Butyl 4-((3-(1-(2,6-dioxopiperidin-3-yl)-3-methyl-2-oxo-2,3-dihydro-1H-benzo[d]-imidazol-4-yl)prop-2-yn-1-yl)oxy)piperidine-1-carboxylate was converted to the title compound by proceeding analogously as described in Example 10, Steps 4-8 above to provide the title compound. MS (ES, m/z): [M+1]⁺=776.4

### Example 17

### Synthesis of 5-((3-(3-((4-((8-cyclopentyl-7-oxo-7,8-dihydropyrido[2,3-d]pyrimidin-2-yl)-amino)piperidin-1-yl)sulfonyl)phenoxy)azetidin-1-yl)methyl)-2-(2,6-dioxopiperidin-3-yl)-isoindoline-1,3-dione

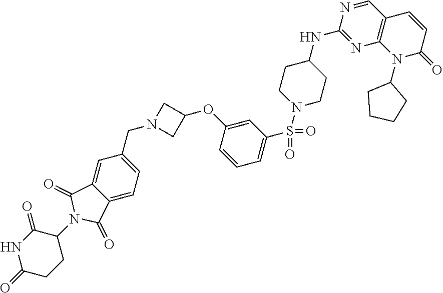 [SMILES: O=C1CCC(N2C(=O)c3ccc(CN4CC(Oc5cccc(S(=O)(=O)N6CCC(Nc7ncc8ccc(=O)n(C9CCCC9)c8n7)CC6)c5)C4)cc3C2=O)C(=O)N1]

### Step 1: tert-butyl (1-((3-((1-((2-(2,6-dioxopiperidin-3-yl)-1,3-dioxoisoindolin-5-yl)methyl)-azetidin-3-yl)oxy)phenyl)sulfonyl)piperidin-4-yl)carbamate

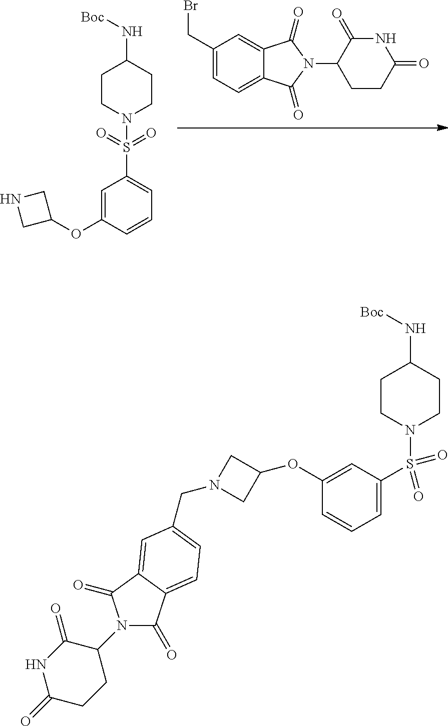 [SMILES: CC(C)(C)OC(=O)NC1CCN(S(=O)(=O)c2cccc(OC3CNC3)c2)CC1 ; O=C1CCC(N2C(=O)c3ccc(CBr)cc3C2=O)C(=O)N1 ; CC(C)(C)OC(=O)NC1CCN(S(=O)(=O)c2cccc(OC3CN(Cc4ccc5c(c4)C(=O)N(C4CCC(=O)NC4=O)C5=O)C3)c2)CC1]

Proceeding analogously as described in Example 8, Step 5 above, but using tert-butyl (1-((3-(azetidin-3-yloxy)phenyl)sulfonyl)piperidin-4-yl)carbamate and 5-(bromomethyl)-2-(2,6-dioxopiperidin-3-yl)isoindoline-1,3-dione provided the title compound.

### Step 2: 5-((3-(3-((4-aminopiperidin-1-yl)sulfonyl)phenoxy)azetidin-1-yl)methyl)-2-(2,6-dioxopiperidin-3-1 isoindoline-1,3-dione

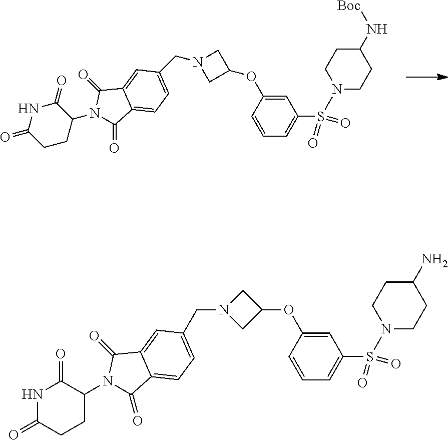 [SMILES: CC(C)(C)OC(=O)NC1CCN(S(=O)(=O)c2cccc(OC3CN(Cc4ccc5c(c4)C(=O)N(C4CCC(=O)NC4=O)C5=O)C3)c2)CC1 ; NC1CCN(S(=O)(=O)c2cccc(OC3CN(Cc4ccc5c(c4)C(=O)N(C4CCC(=O)NC4=O)C5=O)C3)c2)CC1]

Proceeding analogously as described in Example 8, Step 6 above, but using (1-((3-((1-((2-(2,6-dioxopiperidin-3-yl)-1,3-dioxoisoindolin-5-yl)methyl)azetidin-3-yl)oxy)phenyl)sulfonyl)piperidin-4-yl)carbamate and TFA provided the title compound.

### Step 3: 5-((3-(3-((4-((8-cyclopentyl-7-oxo-7,8-dihydropyrido[2,3-d]pyrimidin-2-yl)amino)piperidin-1-yl)sulfonyl)phenoxy)azetidin-1-yl)methyl)-2-(2,6-dioxopiperidin-3-yl)isoindoline-1,3-dione

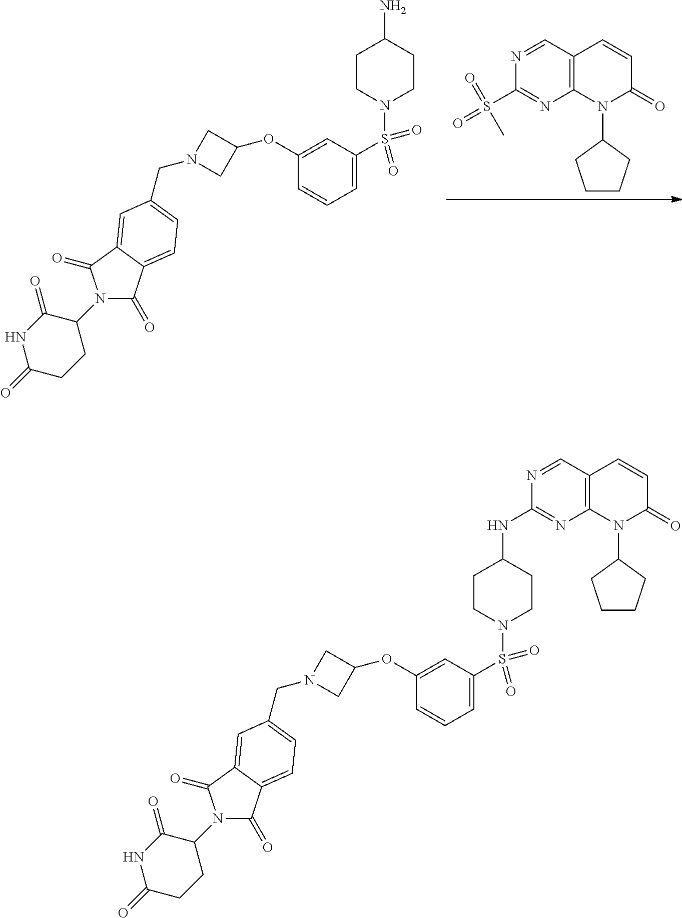

Proceeding analogously as described in Example 8, Step 7 above, but using 5-((3-(3-((4-aminopiperidin-1-yl)sulfonyl)phenoxy)azetidin-1-yl)methyl)-2-(2,6-dioxopiperidin-3-yl)isoindoline-1,3-dione and 8-cyclopentyl-2-(methylsulfonyl)pyrido[2,3-d]pyrimidin-7 (8H)-one provided the title compound. MS (ES, m/z): [M+1]⁺=795.4.

### Example 18

### Synthesis of 4-((8-cyclopentyl-7-oxo-7,8-dihydropyrido[2,3-d]pyrimidin-2-yl)amino)-N-(2-(3-(1-(2,6-dioxopiperidin-3-yl)-3-methyl-2-oxo-2,3-dihydro-1H-benzo[d]imidazol-4-yl)propoxy)ethyl)piperidine-1-sulfonamide

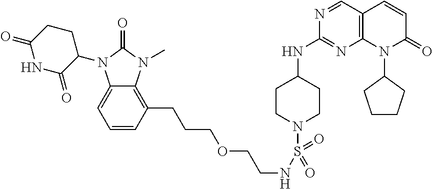 [SMILES: Cn1c(=O)n(C2CCC(=O)NC2=O)c2cccc(CCCOCCNS(=O)(=O)N3CCC(Nc4ncc5ccc(=O)n(C6CCCC6)c5n4)CC3)c21]

### Step 1: tert-butyl (2-(prop-2-yn-1-yloxy)ethyl)carbamate

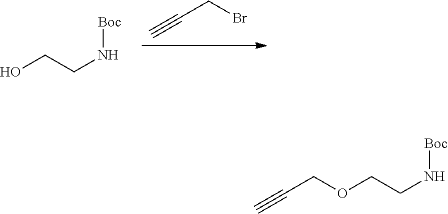

Proceeding analogously as described in Example 10, Step 2 above, but using tert-butyl (2-hydroxyethyl)carbamate and 3-bromoprop-1-yne provided the title compound.

### Step 2: tert-butyl (2-((3-(1-(2,6-dioxopiperidin-3-yl)-3-methyl-2-oxo-2,3-dihydro-1H-benzo[d]imidazol-4-yl)prop-2-yn-1-yl)oxy)ethyl)carbamate

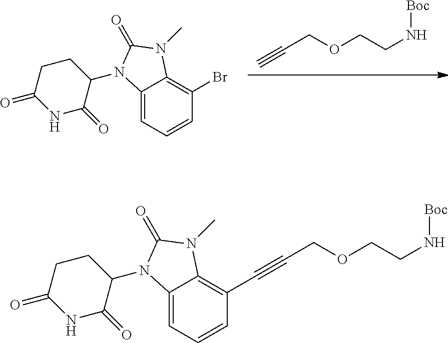

Proceeding analogously as described in Example 10, Step 3 above, but using 3-(4-bromo-3-methyl-2-oxo-2,3-dihydro-1H-benzo[d]imidazol-1-yl)piperidine-2,6-dione and tert-butyl (2-(prop-2-yn-1-yloxy)ethyl)carbamate provided the title compound.

### Step 3: tert-butyl (2-(3-(1-(2,6-dioxopiperidin-3-yl)-3-methyl-2-oxo-2,3-dihydro-1H-benzo[d]imidazol-4-yl)propoxy)ethyl)carbamate

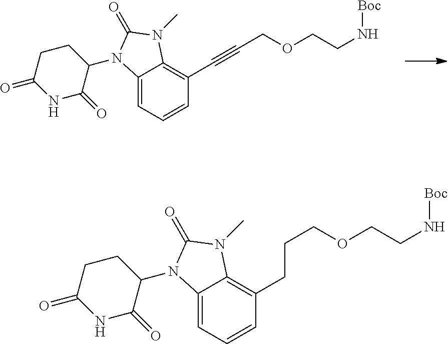 [SMILES: Cn1c(=O)n(C2CCC(=O)NC2=O)c2cccc(C#CCOCCNC(=O)OC(C)(C)C)c21 ; Cn1c(=O)n(C2CCC(=O)NC2=O)c2cccc(CCCOCCNC(=O)OC(C)(C)C)c21]

Proceeding analogously as described in Example 10, Step 4 above, but using tert-butyl (2-((3-(1-(2,6-dioxopiperidin-3-yl)-3-methyl-2-oxo-2,3-dihydro-1H-benzo[d]imidazol-4-yl)prop-2-yn-1-yl)oxy)ethyl)carbamate provided the title compound.

### Step 4: 3-(4-(3-(2-aminoethoxy)propyl)-3-methyl-2-oxo-2,3-dihydro-1H-benzo[d]imidazol-1-yl)piperidine-2,6-dione

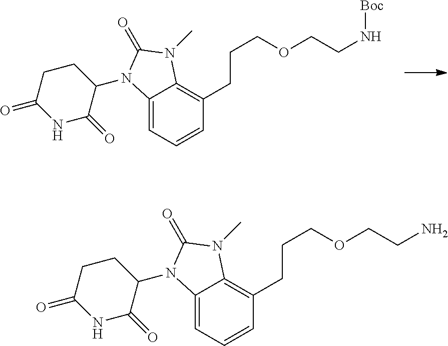 [SMILES: Cn1c(=O)n(C2CCC(=O)NC2=O)c2cccc(CCCOCCNC(=O)OC(C)(C)C)c21 ; Cn1c(=O)n(C2CCC(=O)NC2=O)c2cccc(CCCOCCN)c21]

Proceeding analogously as described in Example 10, Step 5 above, but using tert-butyl (2-(3-(1-(2,6-dioxopiperidin-3-yl)-3-methyl-2-oxo-2,3-dihydro-1H-benzo[d]imidazol-4-yl)propoxy)ethyl)carbamate provided the title compound.

### Step 5: tert-butyl (1-(N-(2-(3-(1-(2,6-dioxopiperidin-3-yl)-3-methyl-2-oxo-2,3-dihydro-1H-benzo[d]imidazol-4-yl)propoxy)ethyl)sulfamoyl)piperidin-4-yl carbamate

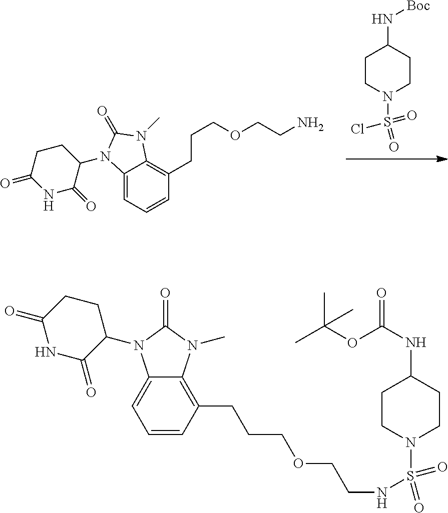 [SMILES: Cn1c(=O)n(C2CCC(=O)NC2=O)c2cccc(CCCOCCN)c21 ; CC(C)(C)OC(=O)NC1CCN(S(=O)(=O)Cl)CC1 ; Cn1c(=O)n(C2CCC(=O)NC2=O)c2cccc(CCCOCCNS(=O)(=O)N3CCC(NC(=O)OC(C)(C)C)CC3)c21]

Proceeding analogously as described in Example 10, Step 6 above, but using 3-(4-(3-(2-aminoethoxy)propyl)-3-methyl-2-oxo-2,3-dihydro-1H-benzo[d]imidazol-1-yl)piperidine-2,6-dione and tert-butyl (1-(chlorosulfonyl)piperidin-4-yl)carbamate provided the title compound.

### Step 6: 4-amino-N-(2-(3-(1-(2,6-dioxopiperidin-3-yl)-3-methyl-2-oxo-2,3-dihydro-1H-benzo[d]imidazol-4-yl)propoxy)ethyl)piperidine-1-sulfonamide

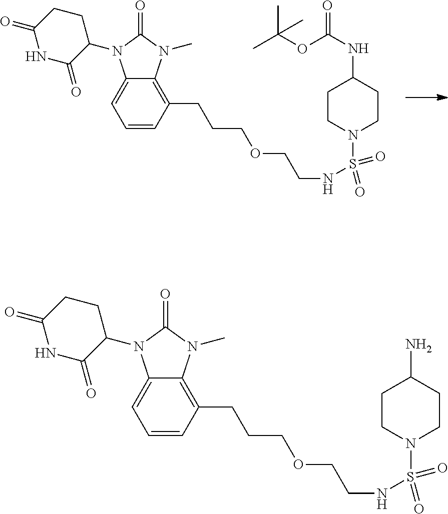 [SMILES: Cn1c(=O)n(C2CCC(=O)NC2=O)c2cccc(CCCOCCNS(=O)(=O)N3CCC(NC(=O)OC(C)(C)C)CC3)c21 ; Cn1c(=O)n(C2CCC(=O)NC2=O)c2cccc(CCCOCCNS(=O)(=O)N3CCC(N)CC3)c21]

Proceeding analogously as described in Example 10, Step 7 above, but using tert-butyl (1-(N-(2-(3-(1-(2,6-dioxopiperidin-3-yl)-3-methyl-2-oxo-2,3-dihydro-1H-benzo[d]imidazol-4-yl)propoxy)ethyl)sulfamoyl)piperidin-4-yl)carbamate provided the title compound.

### Step 7: 4-((8-cyclopentyl-7-oxo-7,8-dihydropyrido[2,3-d]pyrimidin-2-yl)amino)-N-(2-(3-(1-(2,6-dioxopiperidin-3-yl)-3-methyl-2-oxo-2,3-dihydro-1H-benzo[d]imidazol-4-yl)propoxy)ethyl)piperidine-1-sulfonamide

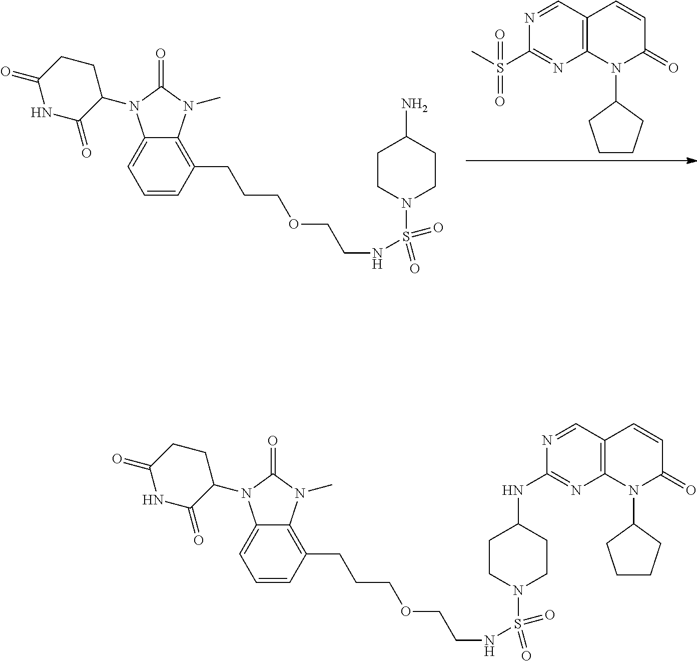 [SMILES: Cn1c(=O)n(C2CCC(=O)NC2=O)c2cccc(CCCOCCNS(=O)(=O)N3CCC(N)CC3)c21 ; CS(=O)(=O)c1ncc2ccc(=O)n(C3CCCC3)c2n1 ; Cn1c(=O)n(C2CCC(=O)NC2=O)c2cccc(CCCOCCNS(=O)(=O)N3CCC(Nc4ncc5ccc(=O)n(C6CCCC6)c5n4)CC3)c21]

Proceeding analogously as described in Example 10, Step 8 above, but using 4-amino-N-(2-(3-(1-(2,6-dioxopiperidin-3-yl)-3-methyl-2-oxo-2,3-dihydro-1H-benzo[d]imidazol-4-yl)propoxy)ethyl)piperidine-1-sulfonamide and 8-cyclopentyl-2-(methylsulfonyl)pyrido[2,3-d]pyrimidin-7 (8H)-one provided the title compound. MS (ES, m/z): [M+1]⁺=736.4.

### Example 19

### Synthesis of 4-((8-cyclopentyl-7-oxo-7,8-dihydropyrido[2,3-d]pyrimidin-2-yl)amino)-N-(3-(3-(1-(2,6-dioxopiperidin-3-yl)-3-methyl-2-oxo-2,3-dihydro-1H-benzo[d]-170-imidazole-4-yl)propoxy)propyl)-N-methylpiperidine-1-sulfonamide

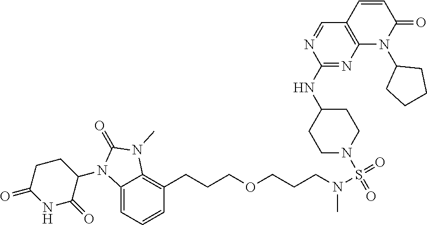 [SMILES: CN(CCCOCCCc1cccc2c1n(C)c(=O)n2C1CCC(=O)NC1=O)S(=O)(=O)N1CCC(Nc2ncc3ccc(=O)n(C4CCCC4)c3n2)CC1]

Proceeding analogously as described in Example 18, Steps 1-7, above but using tert-butyl (3-hydroxypropyl)carbamate provided the title compound. MS (ES, m/z): [M+1]⁺=764.4.

### Example 20

### Synthesis of 4-((8-cyclopentyl-7-oxo-7,8-dihydropyrido[2,3-d]pyrimidin-2-yl)amino)-N-(1-((1-(2,6-dioxopiperidin-3-yl)-3-methyl-2-oxo-2,3-dihydro-1H-benzo[d]imidazol-4-yl)methyl)piperidin-4-yl)-N-methylpiperidine-1-sulfonamide

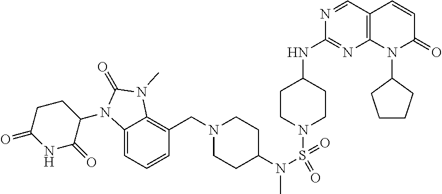 [SMILES: CN(C1CCN(Cc2cccc3c2n(C)c(=O)n3C2CCC(=O)NC2=O)CC1)S(=O)(=O)N1CCC(Nc2ncc3ccc(=O)n(C4CCCC4)c3n2)CC1]

### Step 1: 1-(2,6-dioxopiperidin-3-yl)-3-methyl-2-oxo-2,3-dihydro-1H-benzo[d]imidazole-4-carbaldehyde

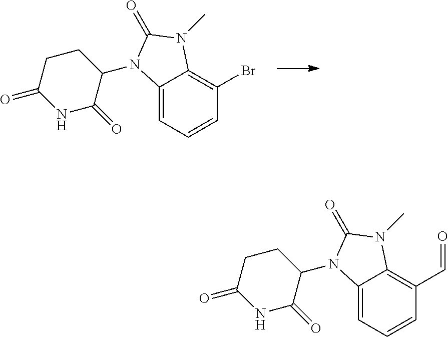 [SMILES: Cn1c(=O)n(C2CCC(=O)NC2=O)c2cccc(Br)c21 ; Cn1c(=O)n(C2CCC(=O)NC2=O)c2cccc(C=O)c21]

A mixture of 3-(4-bromo-3-methyl-2-oxo-2,3-dihydro-1H-benzo[d]imidazol-1-yl)-piperidine-2,6-dione (700 mg, 2.07 mmol, 1.00 eq.), TEA (630 mg, 6.23 mmol, 3.01 eq.), Pd(dppf)Cl₂ (230.6 mg, 0.32 mmol, 0.15 eq.), Et₃SiH (733 mg, 6.30 mmol, 3.04 eq.) in DMF (10 mL) was stirred at 80° C. under 15 psi carbon monoxide atmosphere overnight. The reaction mixture was diluted with water and then extracted with EtOAc. The organic layer was washed with brine, dried over Na₂SO₄, filtered and then concentrated. The residue was purified by chromatograph on silica gel (DCM/MeOH=20/1) to give the title compound (600 mg, 100%) as a yellow oil.

### Step 2: tert-butyl (1-((1-(2,6-dioxopiperidin-3-yl)-3-methyl-2-oxo-2,3-dihydro-1H-benzo[d]imidazol-4-yl)methyl)piperidin-4-yl)(methyl)carbamate

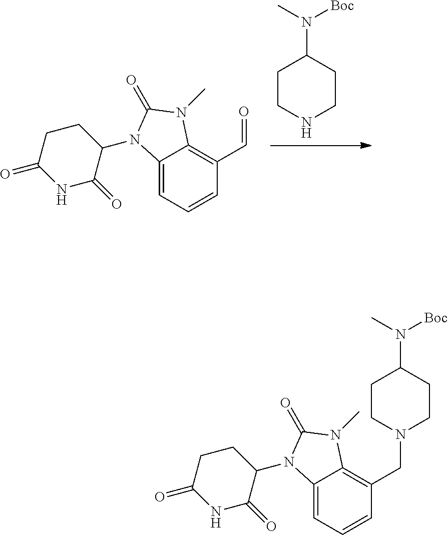

A mixture of 1-(2,6-dioxopiperidin-3-yl)-3-methyl-2-oxo-2,3-dihydro-1H-benzo[d]-imidazole-4-carbaldehyde (360 mg, 1.25 mmol, 1.00 eq.), tert-butyl N-methyl (piperidin-4-yl)carbamate (403 mg, 1.88 mmol, 1.50 eq.) in THF/DMF=2/1 (5 mL) was stirred at RT for 2h. NaBH(OAC)₃ (413 mg, 1.95 mmol, 1.60 eq.) was added at RT. After the reaction was complete, the reaction mixture was diluted with water and then extracted with EtOAc. The organic layer was washed with brine, dried over Na₂SO₄ and then concentrated. The residue was purified by Prep-HPLC to give the title compound (80 mg, 12.8%) as a yellow solid.

### Step 3: 4-((8-cyclopentyl-7-oxo-7,8-dihydropyrido[2,3-d]pyrimidin-2-yl)amino)-N-(1-((1-(2,6-dioxopiperidin-3-yl)-3-methyl-2-oxo-2,3-dihydro-1H-benzo[d]imidazol-4-yl)methyl)piperidin-4-yl)-N-methylpiperidine-1-sulfonamide

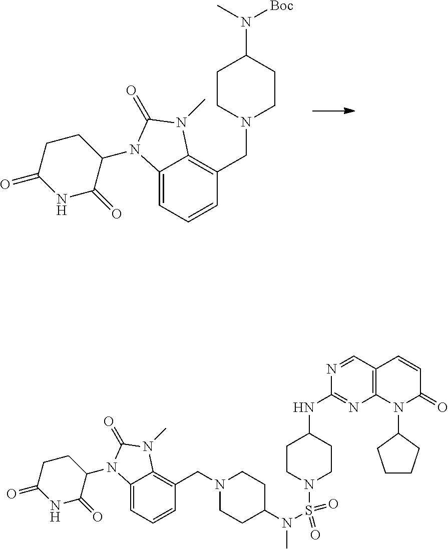 [SMILES: CN(C(=O)OC(C)(C)C)C1CCN(Cc2cccc3c2n(C)c(=O)n3C2CCC(=O)NC2=O)CC1 ; CN(C1CCN(Cc2cccc3c2n(C)c(=O)n3C2CCC(=O)NC2=O)CC1)S(=O)(=O)N1CCC(Nc2ncc3ccc(=O)n(C4CCCC4)c3n2)CC1]

tert-Butyl (1-((1-(2,6-dioxopiperidin-3-yl)-3-methyl-2-oxo-2,3-dihydro-1H-PGP-477C3 benzo[d]imidazol-4-yl)methyl)piperidin-4-yl)(methyl)carbamate was converted to the title compound by proceeding analogously as described in Example 10, Steps 5-8 above. MS (ES, m/z): [M+1]⁺=761.4.

### Example 21

### Synthesis of 4-((8-cyclopentyl-7-oxo-7,8-dihydropyrido[2,3-d]pyrimidin-2-yl)amino)-N-(2-(2-(2-(2-((2-(2,6-dioxopiperidin-3-yl)-1,3-dioxoisoindolin-4-yl)amino)ethoxy)ethoxy)-ethoxy)ethyl)piperidine-1-sulfonamide

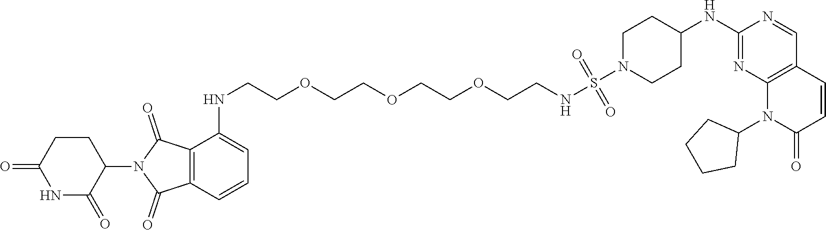 [SMILES: O=C1CCC(N2C(=O)c3cccc(NCCOCCOCCOCCNS(=O)(=O)N4CCC(Nc5ncc6ccc(=O)n(C7CCCC7)c6n5)CC4)c3C2=O)C(=O)N1]

### Step 1: 4-((8-cyclopentyl-7-oxo-7,8-dihydropyrido[2,3-d]pyrimidin-2-yl)amino)-N-(2-(2-(2-(2-((2-(2,6-dioxopiperidin-3-yl)-1,3-dioxoisoindolin-4-yl)amino)ethoxy)ethoxy)ethoxy)ethyl)piperidine-1-sulfonamid

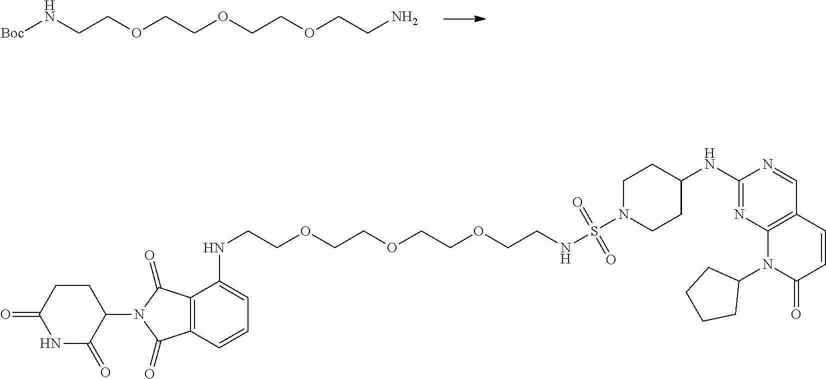 [SMILES: CC(C)(C)OC(=O)NCCOCCOCCOCCN ; O=C1CCC(N2C(=O)c3cccc(NCCOCCOCCOCCNS(=O)(=O)N4CCC(Nc5ncc6ccc(=O)n(C7CCCC7)c6n5)CC4)c3C2=O)C(=O)N1]

Proceeding analogously as described in Example 9, Steps 1-6 above, but using tert-butyl (2-(2-(2-(2-aminoethoxy)ethoxy)ethoxy)ethyl)carbamate instead of tert-butyl (2-(2-(2-aminoethoxy)ethoxy)ethyl)carbamate provided the title compound. MS (ES, m/z): [M+1]⁺=824.4.

### Example 22

### Synthesis of 4-((8-cyclopentyl-7-oxo-7,8-dihydropyrido[2,3-d]pyrimidin-2-yl)amino)-N-(2-((4-((2-(2,6-dioxopiperidin-3-yl)-1,3-dioxoisoindolin-4-yl)(methyl)amino)-benzyl)(methyl)amino)-ethyl)-N-methylpiperidine-1-sulfonamide

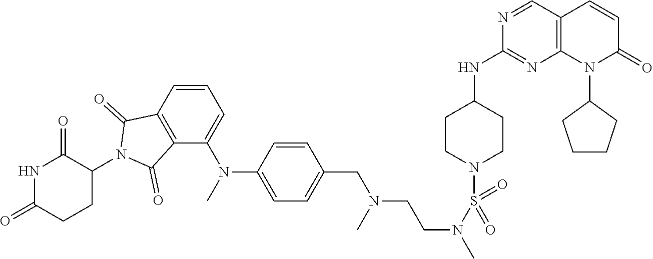 [SMILES: CN(CCN(C)S(=O)(=O)N1CCC(Nc2ncc3ccc(=O)n(C4CCCC4)c3n2)CC1)Cc1ccc(N(C)c2cccc3c2C(=O)N(C2CCC(=O)NC2=O)C3=O)cc1]

### Step 1: dimethyl 3-iodophthalate

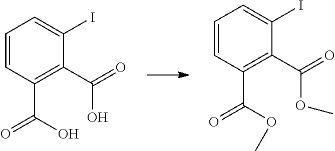

To a stirred mixture of 3-iodophthalic acid (5.00 g, 17.12 mmol, 1.00 eq.), Na₂CO₃ (5.40 g, 50.95 mmol, 2.98 eq.) in DMF (30 mL) was added iodomethane (7.30 g, 51.43 mmol, 3.00 eq.) at RT. The reaction mixture was stirred at 70° C. overnight, cooled, diluted with water, and extracted with EtOAc. The organic layer was washed with brine, dried over Na₂SO₄, filtered, and concentrated. The residue was purified by chromatograph on silica gel (PE/EA=10/1) to give the title compound (4.5 g, 82.1%) as a white solid.

### Step 2: 4-(((tert-butyldimethylsilyl)oxy)methyl)aniline

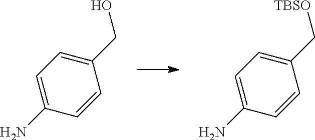 [SMILES: Nc1ccc(CO)cc1 ; CC(C)(C)[Si](C)(C)OCc1ccc(N)cc1]

A mixture of (4-aminophenyl)methanol (2.00 g, 16.24 mmol, 1.00 eq.), DMAP (595 mg, 4.87 mmol, 0.30 eq.), TEA (2.00 g, 19.76 mmol, 1.22 eq.) and TBSCl (2.70 g, 17.91 mmol, 1.10 eq.) in DMF (40 mL) was stirred at RT overnight. The reaction mixture was diluted with water and then extracted with EtOAc. The organic layer was washed with brine, dried over Na₂SO₄, filtered. and then concentrated. The residue was purified by chromatograph on silica gel (PE/EA=10/1) to give the title compound (3.0 g, 77.8%) as a colorless oil.

### Step 3: dimethyl 3-((4-(((tert-butyldimethylsilyl)oxy)methyl)phenyl)amino)phthalate

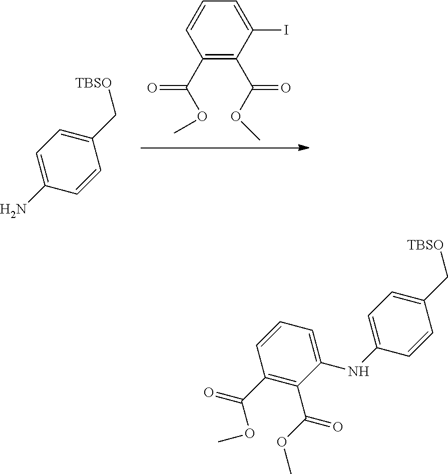

A mixture of 3-iodo-phthalic acid dimethyl ester (3.00 g, 9.37 mmol, 1.00 eq.), 4-(tert-butyl-dimethyl-silanyloxymethyl)-phenylamine (2.67 g, 11.25 mmol, 1.20 eq.), Pd₂(dba)₃ (436 mg, 0.48 mmol, 0.051 eq.), Cs₂CO₃ (6.11 g, 18.75 mmol, 2.00 eq.), BINAP (143 mg, 0.23 mmol, 0.025 eq.) in toluene (30.0 mL) was stirred at 120° C. overnight under nitrogen atmosphere. The reaction mixture was cooled, concentrated and the residue was purified by chromatograph on silica gel (PE/EA=10/1) to give the title compound (1.50 g, 37.2%) as a yellow oil.

### Step 4: dimethyl 3-((4-(((tert-butyldimethylsilyl)oxy)methyl)phenyl)methyl)amino)phthalate

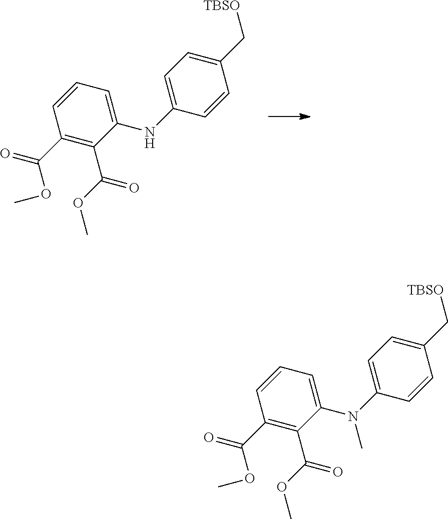 [SMILES: COC(=O)c1cccc(Nc2ccc(CO[Si](C)(C)C(C)(C)C)cc2)c1C(=O)OC ; COC(=O)c1cccc(N(C)c2ccc(CO[Si](C)(C)C(C)(C)C)cc2)c1C(=O)OC]

A mixture of dimethyl 3-((4-(((tert-butyl dimethylsilyl)oxy)methyl)phenyl)-amino)phthalate (1.50 g, 3.49 mmol, 1.00 eq.), iodomethane (991 mg, 6.98 mmol, 2.00 eq.), Cs₂CO₃ (3.41 g, 10.47 mmol, 3.00 eq.) in DMF (30.0 mL) was stirred at 20° C. for 8 h under nitrogen atmosphere. The reaction mixture was diluted with water and extracted with EtOAc. The organic layer was washed with brine, dried over Na₂SO₄, filtered, and then concentrated. The residue was purified by chromatograph on silica gel (PE/EA=5/1) to give the title compound (1.00 g, 64.5%) as a yellow oil.

### Step 5: 3-[(4-hydroxymethyl-phenyl)-methyl-amino]-phthalic acid dimethyl ester

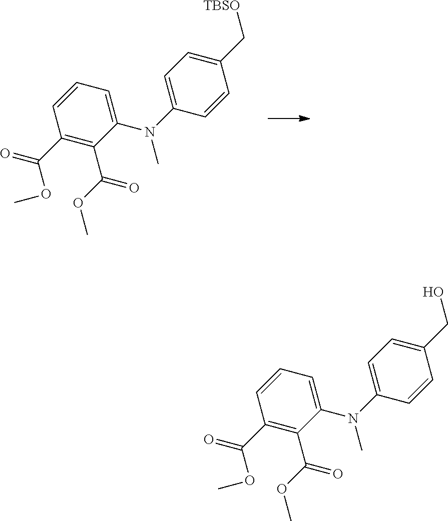 [SMILES: COC(=O)c1cccc(N(C)c2ccc(CO[Si](C)(C)C(C)(C)C)cc2)c1C(=O)OC ; COC(=O)c1cccc(N(C)c2ccc(CO)cc2)c1C(=O)OC]

To a stirred solution of dimethyl 3-((4-(((tert-butyl dimethylsilyl)oxy)methyl)phenyl)-(methyl)amino)phthalate (500 mg, 1.13 mmol, 1.00 eq.) in THF (5.0 mL) was added solution of TBAF in THF (3.0 M, 2.0 mL) at RT. The resulting mixture was stirred at RT for 2 h, diluted with water and then extracted with EtOAc. The organic layer was washed with brine, dried over Na₂SO₄, filtered and then concentrated. The residue was purified by chromatograph on silica gel (PE/EA=2/1) to give the title compound (350 mg, 93.8%) as a yellow oil.

### Step 6: dimethyl 3-((4-formylphenyl)(methyl)amino)phthalate

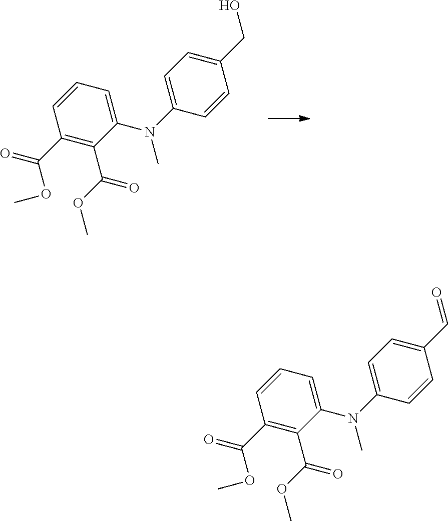 [SMILES: COC(=O)c1cccc(N(C)c2ccc(CO)cc2)c1C(=O)OC ; COC(=O)c1cccc(N(C)c2ccc(C=O)cc2)c1C(=O)OC]

A mixture of 3-[(4-hydroxymethyl-phenyl)methylamino]phthalic acid dimethyl ester (300 mg, 0.91 mmol, 1.00 eq.) and MnO₂ (800 mg, 9.20 mmol, 10.11 eq.) in DCM (10.0 mL) was stirred at RT overnight. The reaction mixture was filtered and concentrated to give the title compound (300 mg) as a yellow oil, which was used for next step without further purification.

### Step 7: dimethyl 3-((4-(((2-((tert-butoxycarbonyl)(methyl)amino)ethyl)(methyl)amino)methyl) phenyl)(methyl)amino)phthalate

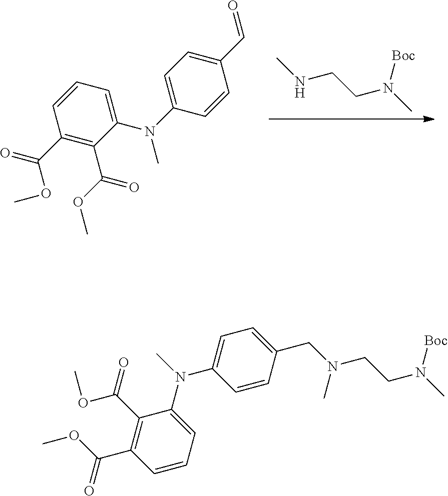

A mixture of dimethyl 3-((4-formylphenyl)(methyl)amino)phthalate (300 mg, 0.92 mmol, 1.00 eq.), methyl-(2-methylamino-ethyl)-carbamic acid tert-butyl ester (205 mg, 1.09 mmol, 1.18 eq.) and a drop of AcOH in DCE (5.0 mL) was stirred at RT for 2 h. NaBH(OAc)₃ (290 mg, 1.37 mmol, 1.49 eq.) was then added and stirred at RT for 4 h. The reaction mixture was concentrated and purified by prep-HPLC to give the title compound (300 mg, 65.2%) as a white solid.

### Step 8: 3-((4-(((2-((tert-butoxycarbonyl)(methyl)amino)ethyl)(methyl)amino)methyl)phenyl) (methyl)amino)phthalic acid

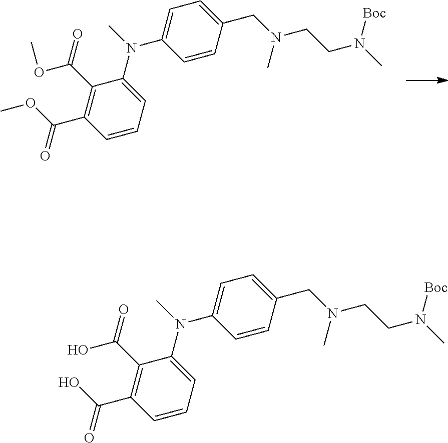

A mixture of dimethyl 3-((4-(((2-((tert-butoxycarbonyl)(methyl)amino)ethyl)(methyl) amino)methyl)phenyl)(methyl)amino)phthalate (250 mg, 0.50 mmol, 1.00 eq.) and NaOH (40 mg, 1.00 mmol, 2.00 eq.) in EtOH/H₂O=2/1 (5.0 mL) was stirred at 80° C. for 5 h. The reaction mixture was concentrated and purified by prep-HPLC to give the title compound (200 mg, 84.0%) as a white solid.

### Step 9: tert-butyl (2-((4-((2-(2,6-dioxopiperidin-3-yl)-1,3-dioxoisoindolin-4-yl)(methyl)amino) benzyl)(methyl)amino)ethyl)(methyl)carbamate

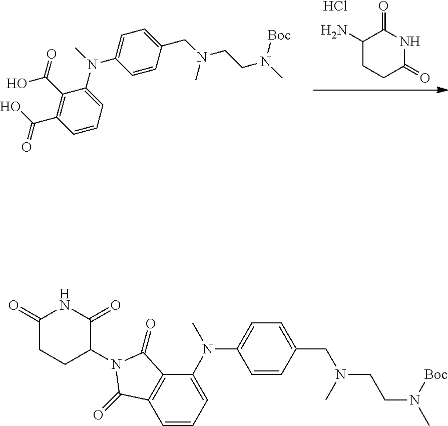 [SMILES: CN(CCN(C)C(=O)OC(C)(C)C)Cc1ccc(N(C)c2cccc(C(=O)O)c2C(=O)O)cc1 ; Cl ; NC1CCC(=O)NC1=O ; CN(CCN(C)C(=O)OC(C)(C)C)Cc1ccc(N(C)c2cccc3c2C(=O)N(C2CCC(=O)NC2=O)C3=O)cc1]

A mixture of 3-((4-(((2-((tert-butoxycarbonyl)(methyl)amino)ethyl)(methyl)amino)methyl) phenyl)(methyl)amino)phthalic acid (120 mg, 0.25 mmol, 1.00 eq.) and 3-aminopiperidine-2,6-dione hydrochloride (41 mg, 0.25 mmol, 1.00 eq.) in pyridine (3.0 mL) was stirred at 100° C. overnight. The reaction mixture was cooled and concentrated. The residue was purified by chromatograph on silica gel (DCM/MeOH=30/1) to give the title compound (60 mg, 44.0%) as a yellow solid.

### Step 10: 4-((8-cyclopentyl-7-oxo-7,8-dihydropyrido[2,3-d]pyrimidin-2-yl)amino)-N-(2-((4-((2-(2,6-dioxopiperidin-3-yl)-1,3-dioxoisoindolin-4-yl)(methyl)amino)benzyl)(methyl)amino)ethyl)-N-methylpiperidine-1-sulfonamide

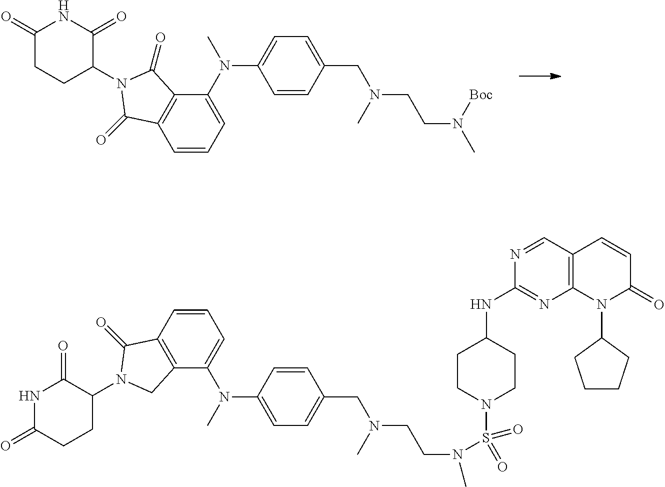 [SMILES: CN(CCN(C)C(=O)OC(C)(C)C)Cc1ccc(N(C)c2cccc3c2C(=O)N(C2CCC(=O)NC2=O)C3=O)cc1 ; CN(CCN(C)S(=O)(=O)N1CCC(Nc2ncc3ccc(=O)n(C4CCCC4)c3n2)CC1)Cc1ccc(N(C)c2cccc3c2CN(C2CCC(=O)NC2=O)C3=O)cc1]

tert-Butyl (2-((4-((2-(2,6-dioxopiperidin-3-yl)-1,3-dioxoisoindolin-4-yl)(methyl)amino) benzyl)(methyl)amino)ethyl)(methyl)carbamate was converted to the title compound by proceeding analogously as described in Example 10, Steps 5-8 above. MS (ES, m/z): [M+1]⁺=839.4.

### Example 23

### Synthesis of 4-((8-cyclopentyl-7-oxo-7,8-dihydropyrido[2,3-d]pyrimidin-2-yl)amino)-N-(3-(4-(1-(2,6-dioxopiperidin-3-yl)-3-methyl-2-oxo-2,3-dihydro-1H-benzo[d]imidazol-4-yl)-piperidin-1-yl)propyl)-N-methylpiperidine-1-sulfonamide

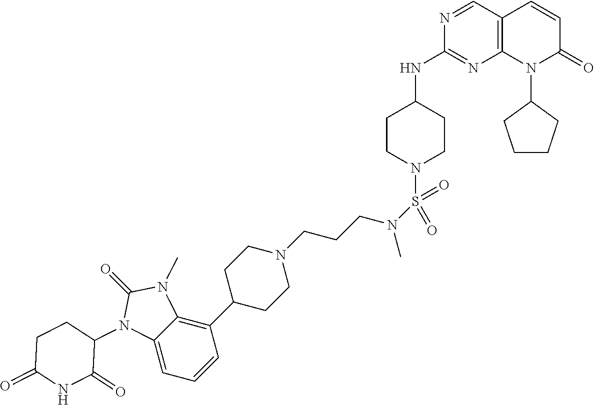 [SMILES: CN(CCCN1CCC(c2cccc3c2n(C)c(=O)n3C2CCC(=O)NC2=O)CC1)S(=O)(=O)N1CCC(Nc2ncc3ccc(=O)n(C4CCCC4)c3n2)CC1]

### Step 1: tert-butyl 4-(1-(2,6-dioxopiperidin-3-yl)-3-methyl-2-oxo-2,3-dihydro-1H-benzo[d]-imidazol-4-yl)-3,6-dihydropyridine-1 (2H)-carboxylate

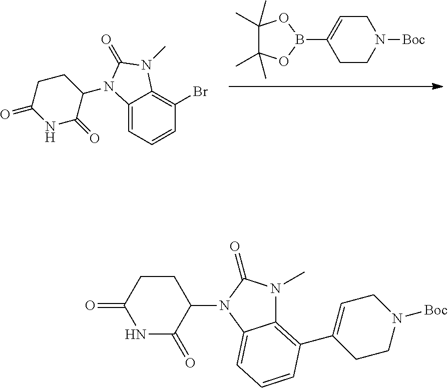 [SMILES: Cn1c(=O)n(C2CCC(=O)NC2=O)c2cccc(Br)c21 ; CC(C)(C)OC(=O)N1CC=C(B2OC(C)(C)C(C)(C)O2)CC1 ; Cn1c(=O)n(C2CCC(=O)NC2=O)c2cccc(C3=CCN(C(=O)OC(C)(C)C)CC3)c21]

A mixture of 3-(4-bromo-3-methyl-2-oxo-2,3-dihydro-1H-benzo[d]imidazol-1-yl)-piperidine-2,6-dione (100 mg, 0.30 mmol, 1.00 eq.), tert-butyl 4-(4,4,5,5-tetramethyl-1,3,2-dioxaborolan-2-yl)-5,6-dihydropyridine-1 (2H)-carboxylate (119 mg, 0.38 mmol, 1.27 eq.), X-phos-G₃ (38 mg, 0.045 mmol, 0.15 eq.), and K₃PO₄ (191 mg, 0.90 mmol, 3.0 eq.) in 1,4-dioxane/H₂O=10/1 (2.2 mL) was stirred at 60° C. for 3 h. The reaction mixture was diluted with water and then extracted with EtOAc. The organic layer was washed with brine, dried over Na₂SO₄, filtered, and then concentrated. The residue was purified by chromatograph on silica gel (DCM/MeOH=20/1) to give the title compound (70 mg, 53.3%) as a brown solid.

### Step 2: tert-butyl 4-(1-(2,6-dioxopiperidin-3-yl)-3-methyl-2-oxo-2,3-dihydro-1H-benzo[d]imidazol-4-yl)piperidine-1-carboxylate

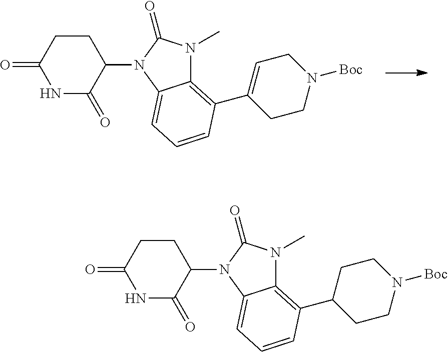 [SMILES: Cn1c(=O)n(C2CCC(=O)NC2=O)c2cccc(C3=CCN(C(=O)OC(C)(C)C)CC3)c21 ; Cn1c(=O)n(C2CCC(=O)NC2=O)c2cccc(C3CCN(C(=O)OC(C)(C)C)CC3)c21]

A mixture of tert-butyl 4-(1-(2,6-dioxopiperidin-3-yl)-3-methyl-2-oxo-2,3-dihydro-1H-benzo[d]imidazol-4-yl)-5,6-dihydropyridine-1 (2H)-carboxylate (70 mg, 0.16 mmol, 1.00 eq.), 10% Pd/C (30 mg) and Pd(OH)₂ (30 mg) in THF (10 mL) was stirred at 50° C. under 50 psi H₂ pressure. The reaction mixture was filtered and then concentrated to give the title compound (60 mg, 87.5%) as a white solid.

### Step 3: 3-(3-methyl-2-oxo-4-(piperidin-4-yl)-2,3-dihydro-1H-benzo[d]imidazol-1-yl)piperidine-2,6-dione TFA salt

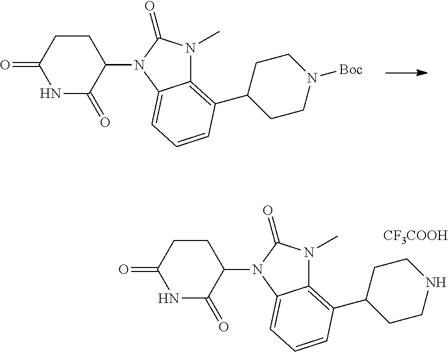 [SMILES: Cn1c(=O)n(C2CCC(=O)NC2=O)c2cccc(C3CCN(C(=O)OC(C)(C)C)CC3)c21 ; Cn1c(=O)n(C2CCC(=O)NC2=O)c2cccc(C3CCNCC3)c21 ; "CF3COOH"]

A mixture of tert-butyl 4-(1-(2,6-dioxopiperidin-3-yl)-3-methyl-2-oxo-2,3-dihydro-1H-benzo[d]imidazol-4-yl)piperidine-1-carboxylate (60 mg, 0.14 mmol, 1.00 eq.) and TFA (0.5 mL) in DCM (2 mL) was stirred at RT for 2 h. The reaction mixture was concentrated to give the title compound (60 mg, 92.9%) as a yellow oil.

### Step 4: tert-butyl (3-(4-(1-(2,6-dioxopiperidin-3-yl)-3-methyl-2-oxo-2,3-dihydro-1H-benzo[d]-imidazol-4-yl)piperidin-1-yl)propyl)(methyl)carbamate

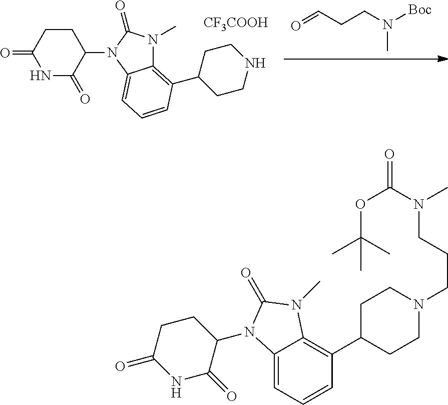 [SMILES: Cn1c(=O)n(C2CCC(=O)NC2=O)c2cccc(C3CCNCC3)c21 ; "CF3COOH" ; CN(CCC=O)C(=O)OC(C)(C)C ; CN(CCCN1CCC(c2cccc3c2n(C)c(=O)n3C2CCC(=O)NC2=O)CC1)C(=O)OC(C)(C)C]

To a stirred mixture of 3-(3-methyl-2-oxo-4-(piperidin-4-yl)-2,3-dihydro-1H-benzo[d]imidazol-1-yl)piperidine-2,6-dione TFA salt (60 mg, 0.13 mmol, 1.00 eq.) in THF (5.0 mL) and DMF (1.0 mL) was added one drop of AcOH. After stirring at RT for 0.5h, tert-butyl methyl(3-oxopropyl)carbamate (63.6 mg, 0.34 mmol, 2.0 eq) was added at RT. The mixture was stirred at 20° C. for 2 h. To the mixture was added NaBH(OAC)3 (72 mg, 0.34 mmol, 2.62 eq.). After stirring at RT overnight, the reaction mixture was diluted with water and then extracted with EtOAc. The organic layer was washed with brine, dried over Na₂SO₄, filtered, and then concentrated. The residue was purified by chromatograph on silica gel (DCM/MeOH=50/1) to give the title compound (100 mg) as a yellow solid.

### Step 5: 4-((8-cyclopentyl-7-oxo-7,8-dihydropyrido[2,3-d]pyrimidin-2-yl)amino)-N-(3-(4-(1-(2,6-dioxopiperidin-3-yl)-3-methyl-2-oxo-2,3-dihydro-1H-benzo[d]imidazol-4-yl)piperidin-1-yl)propyl)-N-methylpiperidine-1-sulfonamide

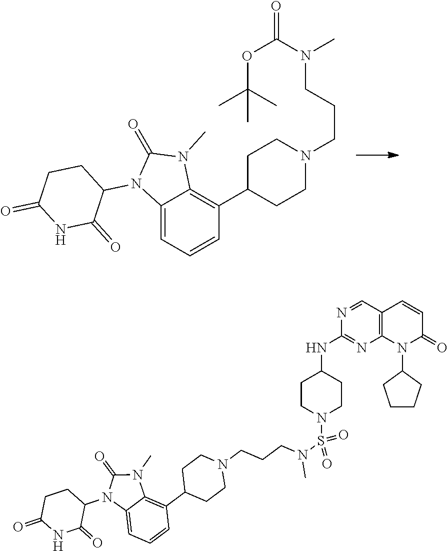 [SMILES: CN(CCCN1CCC(c2cccc3c2n(C)c(=O)n3C2CCC(=O)NC2=O)CC1)C(=O)OC(C)(C)C ; CN(CCCN1CCC(c2cccc3c2n(C)c(=O)n3C2CCC(=O)NC2=O)CC1)S(=O)(=O)N1CCC(Nc2ncc3ccc(=O)n(C4CCCC4)c3n2)CC1]

tert-Butyl (3-(4-(1-(2,6-dioxopiperidin-3-yl)-3-methyl-2-oxo-2,3-dihydro-1H-benzo[d]imidazol-4-yl)piperidin-1-yl)propyl)(methyl)carbamate was converted to the title compound by proceeding analogously as described in Example 10, Steps 5-8 above. MS (ES, m/z): [M+1]⁺=789.4.

### Example 24

### Synthesis of 4-((8-cyclopentyl-7-oxo-7,8-dihydropyrido[2,3-d]pyrimidin-2-yl)amino)-N-(((2R)-4-(3-(1-(2,6-dioxopiperidin-3-yl)-3-methyl-2-oxo-2,3-dihydro-1H-benzo[d]imidazol-4-yl)-propyl)morpholin-2-yl)methyl)-N-methylpiperidine-sulfonamide

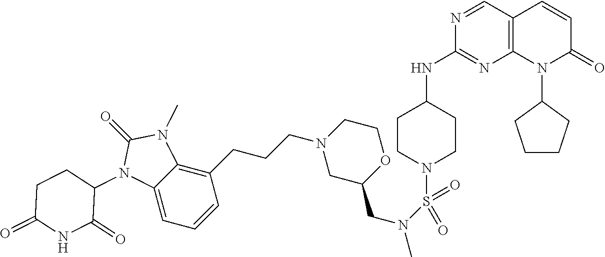 [SMILES: CN(C[C@H]1CN(CCCc2cccc3c2n(C)c(=O)n3C2CCC(=O)NC2=O)CCO1)S(=O)(=O)N1CCC(Nc2ncc3ccc(=O)n(C4CCCC4)c3n2)CC1]

### Step 1: (R)-benzyl 2-(hydroxymethyl)morpholine-4-carboxylate

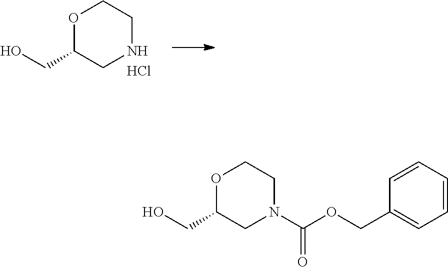 [SMILES: OC[C@H]1CNCCO1 ; Cl ; O=C(OCc1ccccc1)N1CCO[C@@H](CO)C1]

To a stirred mixture of (R)-morpholin-2-ylmethanol hydrochloride (2.0 g, 13.02 mmol, 1.00 eq.) and NaHCO₃ (2.2 g, 26.19 mmol, 2.01 eq.) in THF/H₂O=1/1 (40 mL) was added benzyl chloroformate (2.9 g, 17.00 mmol, 1.31 eq.) at RT. After stirring at 25° C. overnight, the reaction mixture was diluted with water and then extracted with EtOAc. The organic layer was washed with brine, dried over Na₂SO₄, filtered, and then concentrated. The residue was purified by chromatograph on silica gel (PE/EA=1/1) to give the title compound (2.0 g, 61.1%) as a colorless oil.

### Step 2: benzyl (R)-2-(((methylsulfonyl)oxy)methyl)morpholine-4-carboxylate

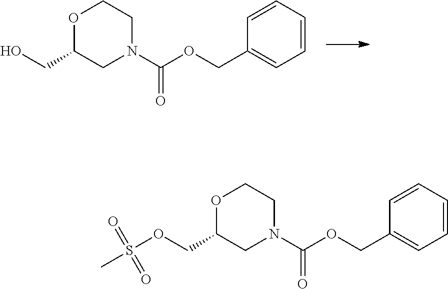 [SMILES: O=C(OCc1ccccc1)N1CCO[C@@H](CO)C1 ; CS(=O)(=O)OC[C@H]1CN(C(=O)OCc2ccccc2)CCO1]

To a stirred mixture of (R)-benzyl 2-(hydroxymethyl)morpholine-4-carboxylate (2.0 g, 7.96 mmol, 1.00 eq.) and TEA (2.4 g, 23.72 mmol, 3.00 eq.) in DCM (30 mL) was added MsCl (1.4 g, 12.22 mmol, 1.54 eq.) slowly at 0° C. After stirring at 0° C. for 2 h, the reaction mixture was quenched with water and then extracted with DCM. The organic layer was washed with brine, dried over Na₂SO₄, filtered, and then concentrated to give the title compound (2.5 g, 95.3%) as a yellow oil, which was used for next step without further purification.

### Step 3: benzyl (S)-2-((methylamino)methyl)morpholine-4-carboxylate

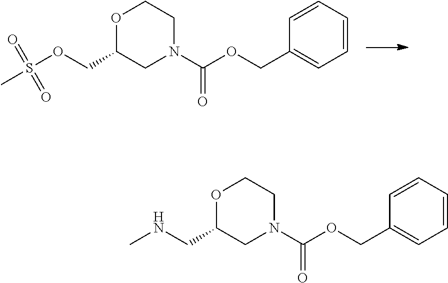 [SMILES: CS(=O)(=O)OC[C@H]1CN(C(=O)OCc2ccccc2)CCO1 ; CNC[C@H]1CN(C(=O)OCc2ccccc2)CCO1]

To a stirred solution of benzyl (R)-2-(((methylsulfonyl)oxy)methyl)morpholine-4-carboxylate (2.0 g, 6.07 mmol, 1.00 eq.) in EtOH (10 mL) was added a solution of methylamine in EtOH (10 mL, 1.0M) at RT. The resulting mixture was stirred at 80° C. overnight, and then concentrated to give the title compound (1.5 g, 93.4%) as a yellow oil.

### Step 4: benzyl (S)-2-(((tert-butoxycarbonyl)(methyl)amino)methyl)morpholine-4-carboxylate

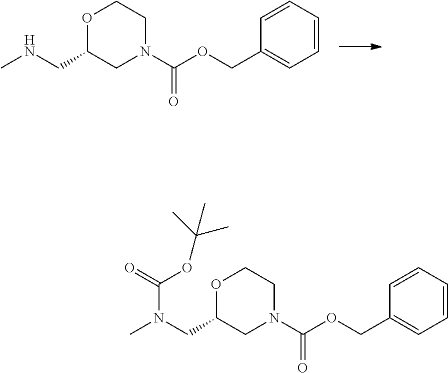 [SMILES: CNC[C@H]1CN(C(=O)OCc2ccccc2)CCO1 ; CN(C[C@H]1CN(C(=O)OCc2ccccc2)CCO1)C(=O)OC(C)(C)C]

To a stirred mixture of benzyl (S)-2-((methylamino)methyl)morpholine-4-carboxylate (1.6 g, 6.05 mmol, 1.00 eq.) and TEA (1.8 g, 17.79 mmol, 2.94 eq.) in DCM (30 mL) was added (Boc)₂O (2.0 g, 9.16 mmol, 1.51 eq.) at RT. After stirring at RT for 2 h, the reaction mixture was concentrated and then purified by chromatograph on silica gel (PE/EA=3/1) to give the title compound (2.0 g, 90.7%) as a white solid.

### Step 5: tert-butyl (R)-methyl(morpholin-2-ylmethyl)carbamate

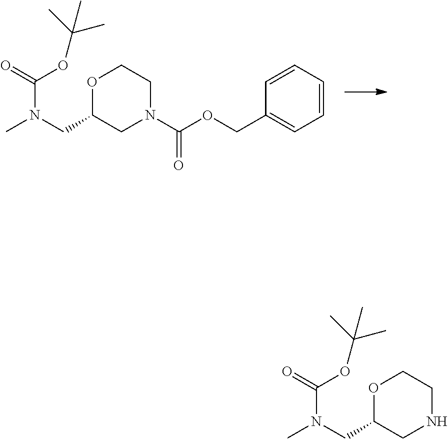 [SMILES: CN(C[C@H]1CN(C(=O)OCc2ccccc2)CCO1)C(=O)OC(C)(C)C ; CN(C[C@H]1CNCCO1)C(=O)OC(C)(C)C]

A mixture of benzyl (S)-2-(((tert-butoxycarbonyl)(methyl)amino)methyl)morpholine-4-carboxylate (2.0 g, 5.49 mmol, 1.00 eq.) and 10% Pd/C (500 mg) in THF (30 mL) was stirred at RT under H₂ overnight. The reaction mixture was filtered and then concentrated to give the title compound (1.1 g, 87.1%) as a yellow oil.

### Step 6: tert-butyl (R)-methyl((4-(prop-2-yn-1-yl)morpholin-2-yl)methyl)carbamate

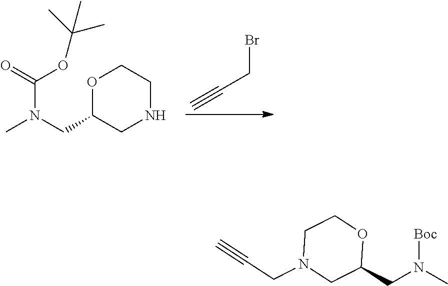

To a stirred mixture of tert-butyl (R)-methyl(morpholin-2-ylmethyl)carbamate (1.3 g, 5.64 mmol, 1.00 eq.) in THF (30 mL) was added NaH (456 mg, 60%, 11.40 mmol, 2.02 eq.) at 0° C. After stirring at RT for 30 min, 3-bromoprop-1-yne (992 mg, 8.34 mmol, 1.48 eq.) was added at RT. After stirring at RT overnight, the reaction mixture was quenched with water and then extracted with EtOAc. The organic layer was washed with brine, dried over Na₂SO₄, filtered and then concentrated. The residue was purified by chromatograph on silica gel (PE/EA=3/1) to give the title compound (1.0 g, 66.1%) as a yellow oil.

### Step 7: 4-((8-cyclopentyl-7-oxo-7,8-dihydropyrido[2,3-d]pyrimidin-2-yl)amino)-N-(((2R)-4-(3-(1-(2,6-dioxopiperidin-3-yl)-3-methyl-2-oxo-2,3-dihydro-1H-benzo[d]imidazol-4-yl)propyl)-morpholin-2-yl)methyl)-N-methylpiperidine-1-sulfonamide

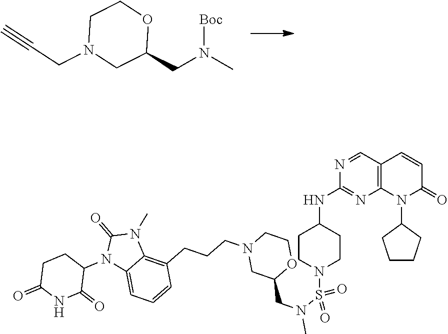 [SMILES: C#CCN1CCO[C@@H](CN(C)C(=O)OC(C)(C)C)C1 ; CN(C[C@H]1CN(CCCc2cccc3c2n(C)c(=O)n3C2CCC(=O)NC2=O)CCO1)S(=O)(=O)N1CCC(Nc2ncc3ccc(=O)n(C4CCCC4)c3n2)CC1]

tert-Butyl (R)-methyl((4-(prop-2-yn-1-yl)morpholin-2-yl)methyl)carbamate was converted to the title compound by proceeding analogously as described in Example 10, Steps 3-8 above using 3-(4-bromo-3-methyl-2-oxo-2,3-dihydro-1H-benzo[d]imidazol-1-yl)-piperidine-2,6-dione.

MS (ES, m/z): [M+1]⁺=805.3.

### Example 25

### Synthesis of 4-((15-((4-((8-cyclopentyl-7-oxo-7,8-dihydropyrido[2,3-d]pyrimidin-2-yl)amino)-piperidin-1-yl)sulfonyl)-3,6,9-trioxa-12-azapentadecyl)amino)-2-(2,6-dioxopiperidin-3-yl)-isoindoline-1,3-dione

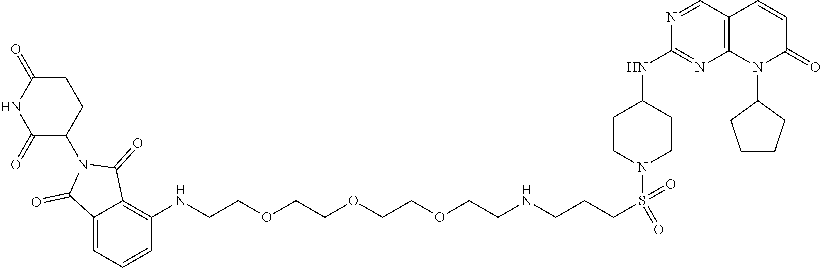 [SMILES: O=C1CCC(N2C(=O)c3cccc(NCCOCCOCCOCCNCCCS(=O)(=O)N4CCC(Nc5ncc6ccc(=O)n(C7CCCC7)c6n5)CC4)c3C2=O)C(=O)N1]

### Step 1: tert-butyl (1-((3-chloropropyl)sulfonyl)piperidin-4-yl)carbamate

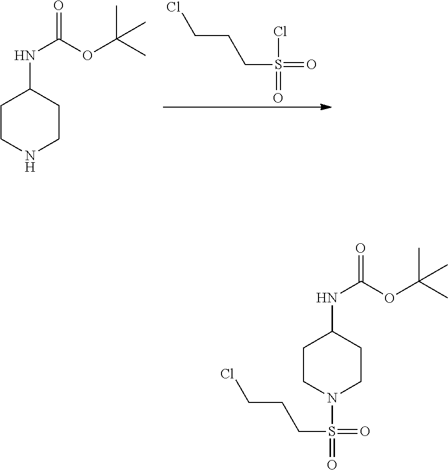

Proceeding analogously as described in Example 8, Step 1 above, but using tert-butyl piperidin-4-ylcarbamate and 3-chloropropane-1-sulfonyl chloride provided the title compound.

### Step 2: 1-((3-chloropropyl)sulfonyl)piperidin-4-amine

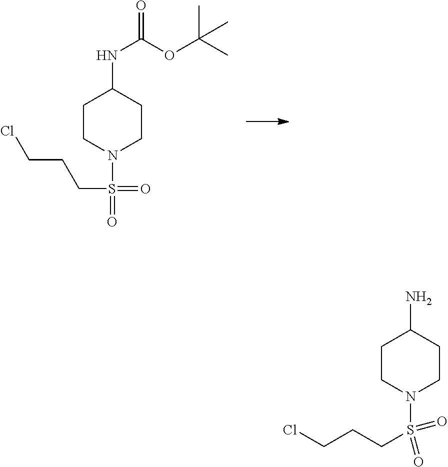 [SMILES: CC(C)(C)OC(=O)NC1CCN(S(=O)(=O)CCCCl)CC1 ; NC1CCN(S(=O)(=O)CCCCl)CC1]

Proceeding analogously as described in Example 8, Step 6 above, but using tert-butyl (1-((3-chloropropyl)sulfonyl)piperidin-4-yl)carbamate provided the title compound.

### Step 3: 2-((1-((3-chloropropyl)sulfonyl)piperidin-4-yl)amino)-8-cyclopentylpyrido[2,3-d]-pyrimidin-7 (8H)-one

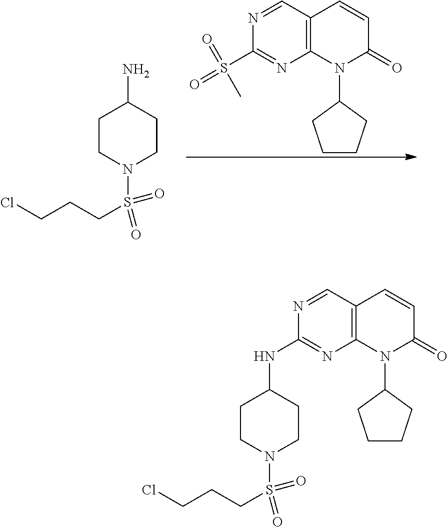 [SMILES: NC1CCN(S(=O)(=O)CCCCl)CC1 ; CS(=O)(=O)c1ncc2ccc(=O)n(C3CCCC3)c2n1 ; O=c1ccc2cnc(NC3CCN(S(=O)(=O)CCCCl)CC3)nc2n1C1CCCC1]

Proceeding analogously as described in Example 8, Step 7 above, but using 1-((3-chloropropyl)sulfonyl)piperidin-4-amine and 8-cyclopentyl-2-(methylsulfonyl)pyrido[2,3-d]-pyrimidin-7 (8H)-one provided the title compound.

### Step 4: tert-butyl (15-((4-((8-cyclopentyl-7-oxo-7,8-dihydropyrido[2,3-d]pyrimidin-2-yl)amino)piperidin-1-yl)sulfonyl)-3,6,9-trioxa-12-azapentadecyl)carbamate

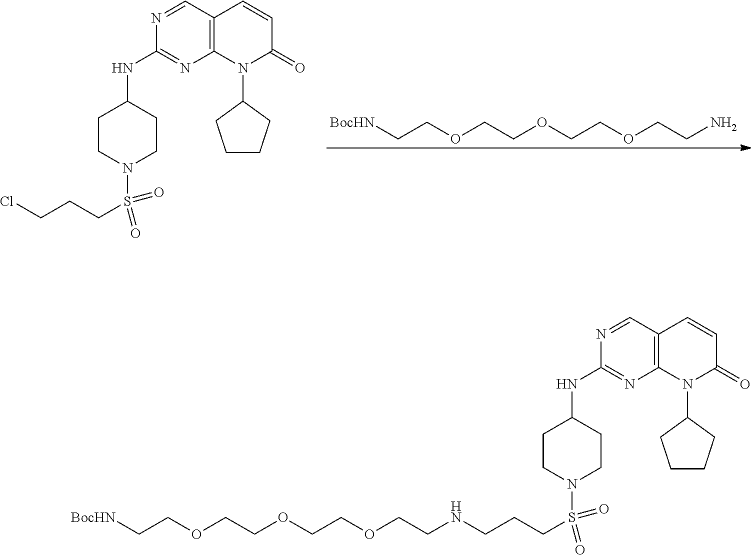 [SMILES: O=c1ccc2cnc(NC3CCN(S(=O)(=O)CCCCl)CC3)nc2n1C1CCCC1 ; CC(C)(C)OC(=O)NCCOCCOCCOCCN ; CC(C)(C)OC(=O)NCCOCCOCCOCCNCCCS(=O)(=O)N1CCC(Nc2ncc3ccc(=O)n(C4CCCC4)c3n2)CC1]

A mixture of 2-((1-((3-chloropropyl)sulfonyl)piperidin-4-yl)amino)-8-cyclopentyl-pyrido[2,3-d]pyrimidin-7 (8H)-one (50 mg, 0.11 mmol, 1.00 eq.), tert-butyl (2-(2-(2-(2-aminoethoxy)ethoxy)ethoxy)ethyl)carbamate (48.2 mg, 0.16 mmol, 1.45 eq.), K₂CO₃ (46 mg, 0.33 mmol, 3.00 eq.) and KI (18.2 mg, 0.11 mmol, 1.00 eq.) in acetonitrile (1 mL) was stirred at 100° C. overnight. The reaction mixture was cooled, diluted with water and then extracted with EtOAc. The organic layer was washed with brine, dried over Na₂SO₄, filtered, and then concentrated to give the title compound (100 mg) as a yellow oil which was used for next step without further purification.

### Step 5: 2-((1-((1-amino-3,6,9-trioxa-12-azapentadecan-15-yl)sulfonyl)piperidin-4-yl)amino)-8-cyclopentylpyrido[2,3-d]pyrimidin-7 (8H)-one

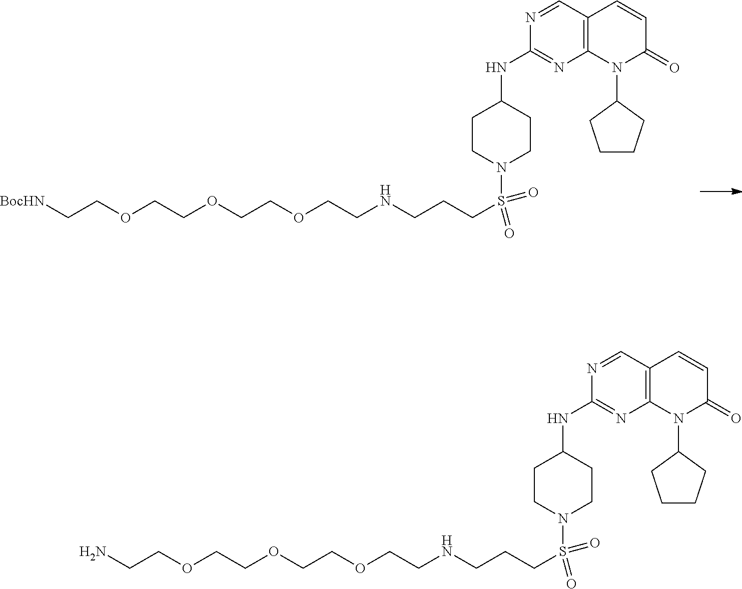 [SMILES: CC(C)(C)OC(=O)NCCOCCOCCOCCNCCCS(=O)(=O)N1CCC(Nc2ncc3ccc(=O)n(C4CCCC4)c3n2)CC1 ; NCCOCCOCCOCCNCCCS(=O)(=O)N1CCC(Nc2ncc3ccc(=O)n(C4CCCC4)c3n2)CC1]

Proceeding analogously as described in Example 8, Step 6 above, but using tert-butyl (15-((4-((8-cyclopentyl-7-oxo-7,8-dihydropyrido[2,3-d]pyrimidin-2-yl)amino)piperidin-1-yl)-sulfonyl)-3,6,9-trioxa-12-azapentadecyl)carbamate provided the title compound.

### Step 6: 2-((1-((1-amino-3,6,9-trioxa-12-azapentadecan-15-yl)sulfonyl)piperidin-4-yl)amino)-8-cyclopentylpyrido[2,3-d]pyrimidin-7 (8H)-one

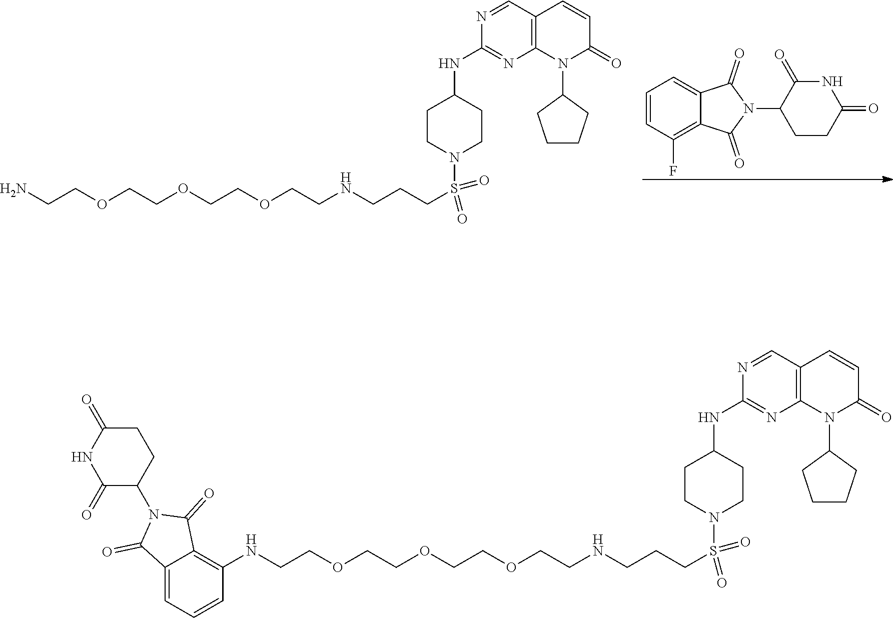

Proceeding analogously as described in Example 9, Step 1 above, but using 2-((1-((1-amino-3,6,9-trioxa-12-azapentadecan-15-yl)sulfonyl)piperidin-4-yl)amino)-8-cyclopentyl-pyrido[2,3-d]pyrimidin-7 (8H)-one and 2-(2,6-dioxopiperidin-3-yl)-4-fluoroisoindoline-1,3-dione provided the title compound. MS (ES, m/z): [M+1]⁺=866.4.

### Example 26

### Synthesis of 14-((4-((8-cyclopentyl-7-oxo-7,8-dihydropyrido[2,3-d]pyrimidin-2-yl)amino)-piperidine)-1-sulfonamido)-N-(2-(2,6-dioxopiperidin-3-yl)-1,3-dioxoisoindolin-4-yl)-3,6,9,12-tetraoxatetradecanamide

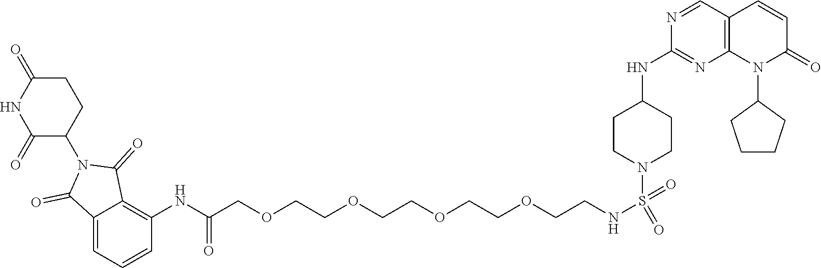 [SMILES: O=C1CCC(N2C(=O)c3cccc(NC(=O)COCCOCCOCCOCCNS(=O)(=O)N4CCC(Nc5ncc6ccc(=O)n(C7CCCC7)c6n5)CC4)c3C2=O)C(=O)N1]

### Step 1: 2,2-dimethyl-4-oxo-3,8,11,14,17-pentaoxa-5-azanonadecan-19-oic acid

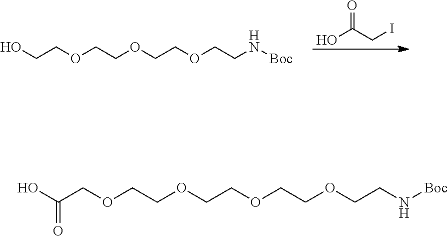 [SMILES: O=C(O)CI ; CC(C)(C)OC(=O)NCCOCCOCCOCCO ; CC(C)(C)OC(=O)NCCOCCOCCOCCOCC(=O)O]

To a stirred solution of tert-butyl (2-(2-(2-(2-hydroxyethoxy)ethoxy)ethoxy)ethyl) carbamate (500 mg, 1.70 mmol, 1.00 eq.) in DMF (2 mL) was added NaH (60% in mineral oil, 204 mg, 5.10 mmol, 3.00 eq.) at 0° C. under nitrogen. After stirring at 0° C. for 1 h, 2-iodoacetic acid (793 mg, 4.26 mmol, 2.51 eq.) was added at 0° C. The resulting mixture was slowly warmed to RT and then stirred at this temperature overnight. This reaction mixture was quenched with H₂O at 0° C., the pH was adjusted to 2-3 with 1 N aqueous HCl and then extracted with EtOAc. The organic layer was washed with brine, dried over Na₂SO₄, filtered, and then concentrated to give the title compound (500 mg, 83.5%) as a yellow oil which was used for next step without further purification.

### Step 2: tert-butyl (14-((2-(2,6-dioxopiperidin-3-yl)-1,3-dioxoisoindolin-4-yl)amino)-14-oxo-3,6,9,12-tetraoxatetradecyl)carbamate

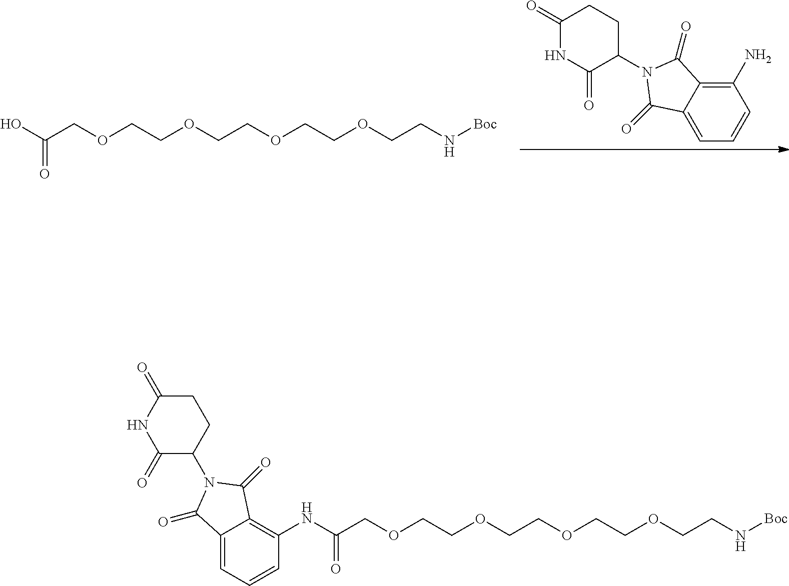 [SMILES: CC(C)(C)OC(=O)NCCOCCOCCOCCOCC(=O)O ; Nc1cccc2c1C(=O)N(C1CCC(=O)NC1=O)C2=O ; CC(C)(C)OC(=O)NCCOCCOCCOCCOCC(=O)Nc1cccc2c1C(=O)N(C1CCC(=O)NC1=O)C2=O]

To a stirred solution of 2,2-dimethyl-4-oxo-3,8,11,14,17-pentaoxa-5-azanonadecan-19-oic acid (372 mg, 1.06 mmol, 2.00 eq.) in THF (6 mL) was added isobutyl chloroformate (109 mg, 0.80 mmol, 1.51 eq.) and N-methylmorpholine (161 mg, 1.59 mmol, 3.00 eq.), followed by a solution of 4-amino-2-(2,6-dioxopiperidin-3-yl)isoindoline-1,3-dione (145 mg, 0.53 mmol, 1.00 eq.) in DMF (2 mL) dropwise at 0° C. The resulting mixture was stirred at 30° C. overnight, quenched with saturated NaHCO₃, extracted with EtOAc. The organic layer was washed with brine, dried over Na₂SO₄, filtered and then concentrated. The residue was purified by column chromatography on silica gel (PE:EA=1:1) to give the title compound (278 mg, 86.8%) as a yellow solid.

### Step 3: 2-(2-(2-(2-aminoethoxy)ethoxy)ethoxy)-N-(2-(2,6-dioxopiperidin-3-yl)-1,3-dioxoisoindolin-4-yl)acetamide 2,2,2-trifluoroacetate

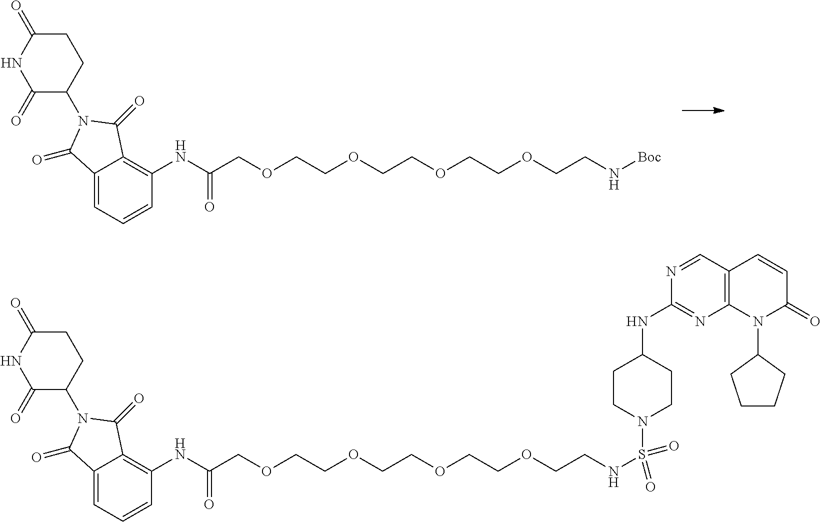 [SMILES: CC(C)(C)OC(=O)NCCOCCOCCOCCOCC(=O)Nc1cccc2c1C(=O)N(C1CCC(=O)NC1=O)C2=O ; O=C1CCC(N2C(=O)c3cccc(NC(=O)COCCOCCOCCOCCNS(=O)(=O)N4CCC(Nc5ncc6ccc(=O)n(C7CCCC7)c6n5)CC4)c3C2=O)C(=O)N1]

tert-Butyl (14-((2-(2,6-dioxopiperidin-3-yl)-1,3-dioxoisoindolin-4-yl)amino)-14-oxo-3,6,9,12-tetraoxatetradecyl)carbamate was converted to the title compound by proceeding analogously as described in Example 10, Steps 5-8 above. MS (ES, m/z): [M+1]⁺=882.3.

### Example 27

### Synthesis of 3-(5-(4-(3-(3-((4-((8-cyclopentyl-7-oxo-7,8-dihydropyrido[2,3-d]pyrimidin-2-yl)amino)-piperidin-1-yl)sulfonyl)phenoxy)azetidin-1-yl)piperidin-1-yl)-1-oxoisoindolin-2-yl)piperidine-2,6-dione

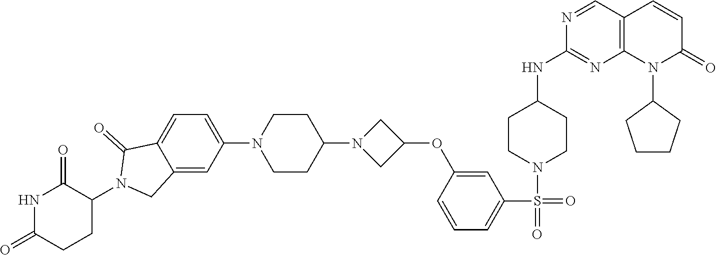 [SMILES: O=C1CCC(N2Cc3cc(N4CCC(N5CC(Oc6cccc(S(=O)(=O)N7CCC(Nc8ncc9ccc(=O)n(C%10CCCC%10)c9n8)CC7)c6)C5)CC4)ccc3C2=O)C(=O)N1]

### tert-butyl (1-((3-(azetidin-3-yloxy)phenyl)sulfonyl)piperidin-4-yl)carbamate

### Step 1: benzyl 4-(3-(3-((4-((tert-butoxycarbonyl)amino)piperidin-1-yl)sulfonyl)phenoxy)-azetidin-1-yl)piperidine-1-carboxylate

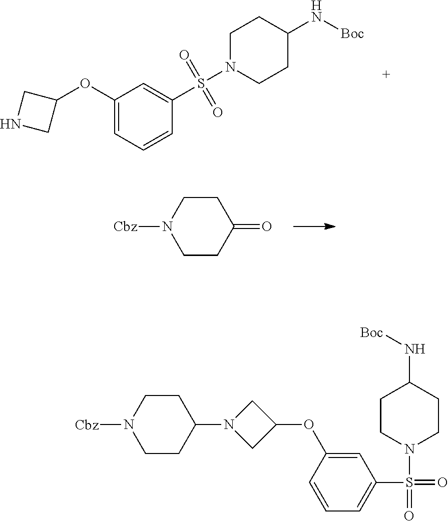 [SMILES: CC(C)(C)OC(=O)NC1CCN(S(=O)(=O)c2cccc(OC3CNC3)c2)CC1 ; O=C1CCN(C(=O)OCc2ccccc2)CC1 ; CC(C)(C)OC(=O)NC1CCN(S(=O)(=O)c2cccc(OC3CN(C4CCN(C(=O)OCc5ccccc5)CC4)C3)c2)CC1]

A solution of tert-butyl (1-((3-(azetidin-3-yloxy)phenyl)sulfonyl)piperidin-4-yl)carbamate (100 mg, 0.24 mmol, 1.00 eq.), benzyl 4-oxopiperidine-1-carboxylate (113 mg, 0.48 mmol, 2.00 eq.) and 1 drop of AcOH in THF (3.0 mL) was stirred at RT for 1h, NaBH(OAc)₃ (102 mg, 0.48 mmol, 2.00 eq.) then was added. The reaction mixture was stirred at RT overnight, diluted with water and then extracted with DCM. The organic layer was concentrated and then purified by silica gel flash column (DCM/MeOH=20/1) to give the title compound (60 mg, 39.6%) as a white solid.

### Step 2: tert-butyl (1-((3-((1-(piperidin-4-yl)azetidin-3-yl)oxy)phenyl)sulfonyl)piperidin-4-yl)carbamate

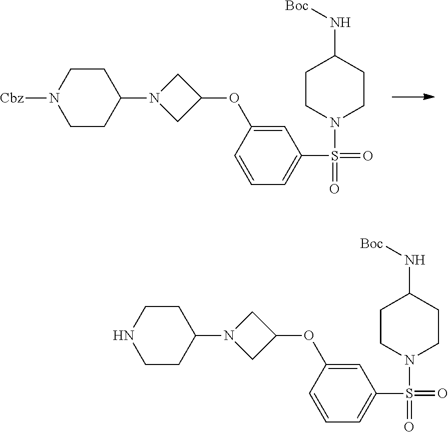 [SMILES: CC(C)(C)OC(=O)NC1CCN(S(=O)(=O)c2cccc(OC3CN(C4CCN(C(=O)OCc5ccccc5)CC4)C3)c2)CC1 ; CC(C)(C)OC(=O)NC1CCN(S(=O)(=O)c2cccc(OC3CN(C4CCNCC4)C3)c2)CC1]

To a stirred solution of benzyl 4-(3-(3-((4-((tert-butoxycarbonyl)amino)piperidin-1-yl)sulfonyl)phenoxy)azetidin-1-yl)piperidine-1-carboxylate (60 mg, 0.095 mmol, 1.00 eq.) in MeOH (10.0 mL) was added 10% Pd/C (20 mg). The resulting mixture was stirred at 45° C. under H₂ atmosphere overnight. The reaction mixture was filtrated and concentrated to give the title compound (38 mg, 81.1%) as a white solid.

### Step 3: tert-butyl (1-((3-((1-(1-(2-(2,6-dioxopiperidin-3-yl)-1-oxoisoindolin-5-yl)piperidin-4-yl)azetidin-3-yl)oxy)phenyl)sulfonyl)piperidin-4-yl)carbamate

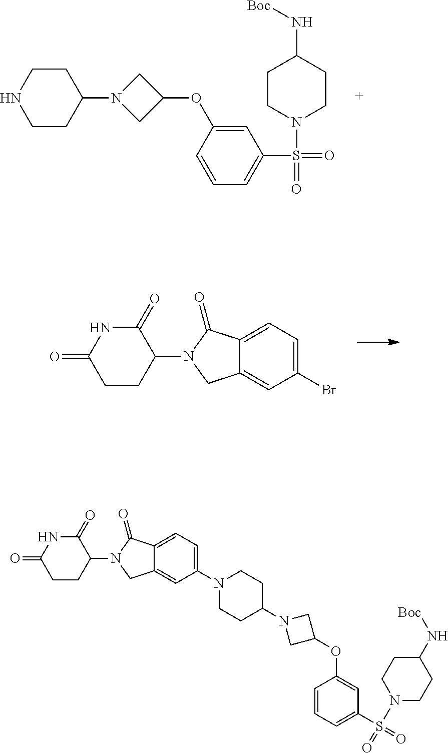 [SMILES: CC(C)(C)OC(=O)NC1CCN(S(=O)(=O)c2cccc(OC3CN(C4CCNCC4)C3)c2)CC1 ; O=C1CCC(N2Cc3cc(Br)ccc3C2=O)C(=O)N1 ; CC(C)(C)OC(=O)NC1CCN(S(=O)(=O)c2cccc(OC3CN(C4CCN(c5ccc6c(c5)CN(C5CCC(=O)NC5=O)C6=O)CC4)C3)c2)CC1]

To a stirred solution of tert-butyl (1-((3-((1-(piperidin-4-yl)azetidin-3-yl)oxy)phenyl) sulfonyl)piperidin-4-yl)carbamate (39.6 mg, 0.080 mmol, 1.00 eq.) in 1,4-dioxane (2.0 mL) was added 3-(5-bromo-1-oxoisoindolin-2-yl)piperidine-2,6-dione (38 mg, 0.12 mmol, 1.50 eq.), Cs₂CO₃ (78 mg, 0.24 mmol, 3.00 eq.), Xantphos (15 mg, 0.027 mmol, 0.34 eq.) and Pd(OAc)₂ (15 mg, 0.067 mmol, 0.84 eq.) under N₂ atmosphere. The resulting mixture was stirred at 100° C. overnight, cooled and then filtered. The filtrate was diluted with water and then extracted with DCM. The organic layer was concentrated and then purified by prep-TLC (DCM/MeOH=10/1) to give the title compound (10 mg, 17.5%) as a yellow solid.

### Step 4: 3-(5-(4-(3-(3-((4-((8-cyclopentyl-7-oxo-7,8-dihydropyrido[2,3-d]pyrimidin-2-yl)amino)piperidin-1-yl)sulfonyl)phenoxy)azetidin-1-yl)piperidin-1-yl)-1-oxoisoindolin-2-yl)piperidine-2,6-dione

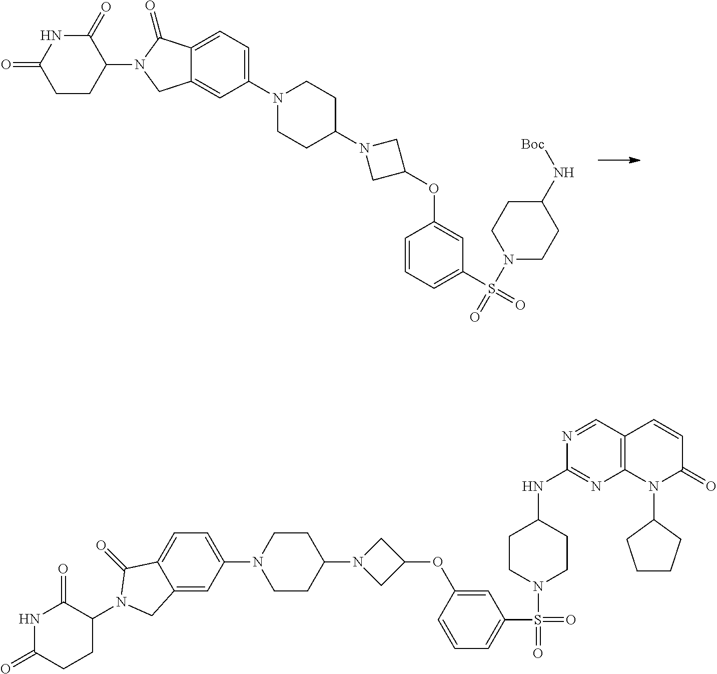 [SMILES: CC(C)(C)OC(=O)NC1CCN(S(=O)(=O)c2cccc(OC3CN(C4CCN(c5ccc6c(c5)CN(C5CCC(=O)NC5=O)C6=O)CC4)C3)c2)CC1 ; O=C1CCC(N2Cc3cc(N4CCC(N5CC(Oc6cccc(S(=O)(=O)N7CCC(Nc8ncc9ccc(=O)n(C%10CCCC%10)c9n8)CC7)c6)C5)CC4)ccc3C2=O)C(=O)N1]

tert-Butyl (1-((3-((1-(1-(2-(2,6-dioxopiperidin-3-yl)-1-oxoisoindolin-5-yl)piperidin-4-yl)azetidin-3-yl)oxy)phenyl)sulfonyl)piperidin-4-yl)carbamate was converted to the title compound by proceeding analogously as described in Example 7, Steps 5-6 above. MS (ES, m/z): [M+1]⁺=850.5.

### Example 28

### Synthesis of 3-(5-(3-(4-(3-((4-((8-cyclopentyl-7-oxo-7,8-dihydropyrido[2,3-d]pyrimidin-2-yl)-amino)piperidin-1-yl)sulfonyl)phenoxy)piperidin-1-yl)azetidin-1-yl)-1-oxoisoindolin-2-yl)-piperidine-2,6-dione

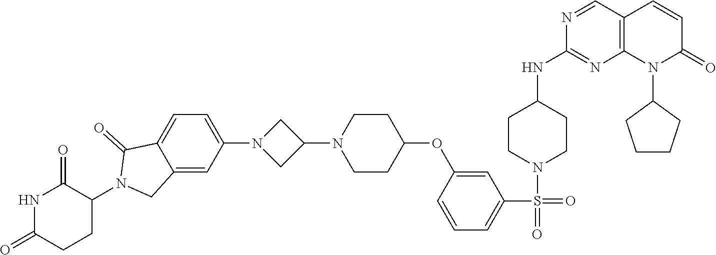 [SMILES: O=C1CCC(N2Cc3cc(N4CC(N5CCC(Oc6cccc(S(=O)(=O)N7CCC(Nc8ncc9ccc(=O)n(C%10CCCC%10)c9n8)CC7)c6)CC5)C4)ccc3C2=O)C(=O)N1]

### Step 1: tert-butyl (1-((3-(piperidin-4-yloxy)phenyl)sulfonyl)piperidin-4-yl)carbamate

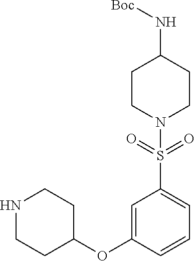 [SMILES: CC(C)(C)OC(=O)NC1CCN(S(=O)(=O)c2cccc(OC3CCNCC3)c2)CC1]

The title compound was prepared by proceeding as described in Example 12, Steps 1 to 6 using 1-benzhydrylpiperidin-4-yl methanesulfonate.

### Step 2: tert-butyl (1-((3-((1-(azetidin-3-yl)piperidin-4-yl)oxy)phenyl)sulfonyl)piperidin-4-yl)-carbamate

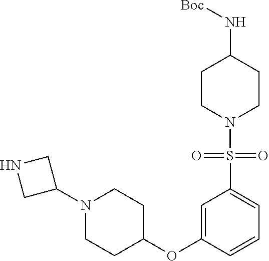 [SMILES: CC(C)(C)OC(=O)NC1CCN(S(=O)(=O)c2cccc(OC3CCN(C4CNC4)CC3)c2)CC1]

tert Butyl (1-((3-(piperidin-4-yloxy)phenyl)sulfonyl)piperidin-4-yl)carbamate was converted to the title compound by proceeding analogously as described in Example 27, Steps 1 and 2 above using benzyl 3-oxoazetidine-1-carboxylate.

### Step 3: 3-(5-(3-(4-(3-((4-((8-cyclopentyl-7-oxo-7,8-dihydropyrido[2,3-d]pyrimidin-2-yl)amino)piperidin-1-yl)sulfonyl)phenoxy)piperidin-1-yl)azetidin-1-yl)-1-oxoisoindolin-2-yl)piperidine-2,6-dione

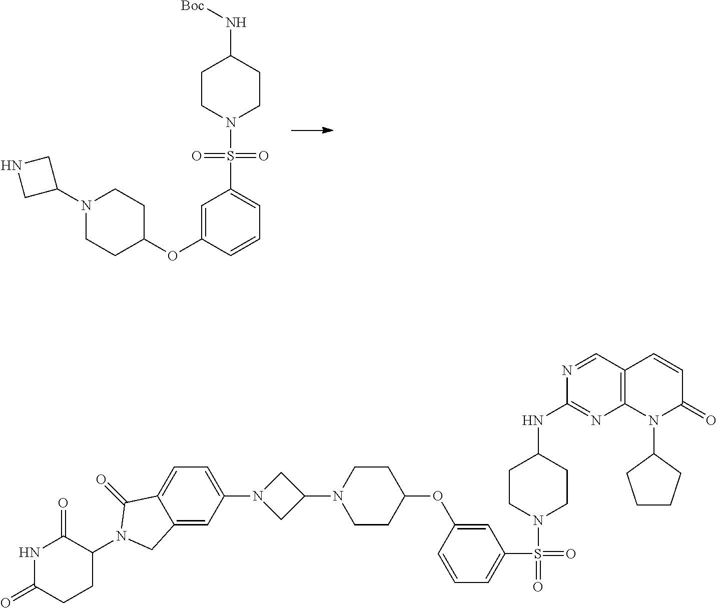 [SMILES: CC(C)(C)OC(=O)NC1CCN(S(=O)(=O)c2cccc(OC3CCN(C4CNC4)CC3)c2)CC1 ; O=C1CCC(N2Cc3cc(N4CC(N5CCC(Oc6cccc(S(=O)(=O)N7CCC(Nc8ncc9ccc(=O)n(C%10CCCC%10)c9n8)CC7)c6)CC5)C4)ccc3C2=O)C(=O)N1]

tert-Butyl (1-((3-((1-(azetidin-3-yl)piperidin-4-yl)oxy)phenyl)sulfonyl)piperidin-4-yl)carbamate was converted to the title compound by proceeding analogously as described in Example 27, Steps 3 and 4 above. MS (ES, m/z): [M+1]⁺=850.5.

### Example 29

### Synthesis of 3-(4-(2-(4-(3-((4-((8-cyclopentyl-7-oxo-7,8-dihydropyrido[2,3-d]pyrimidin-2-yl)amino)piperidin-1-yl)sulfonyl)phenoxy)piperidin-1-yl)ethoxy)-1-oxoisoindolin-2-yl)-piperidine-2,6-dione

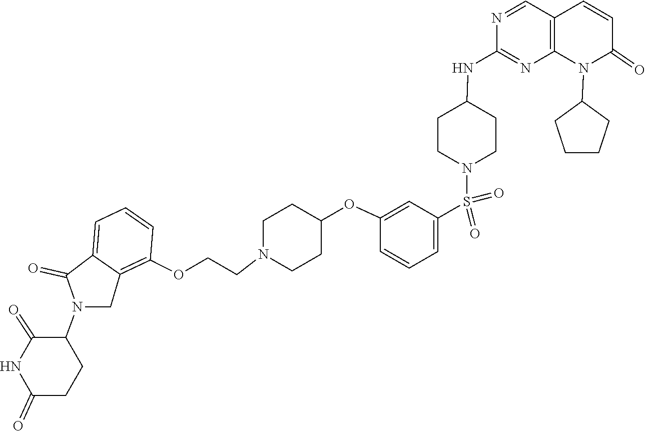 [SMILES: O=C1CCC(N2Cc3c(OCCN4CCC(Oc5cccc(S(=O)(=O)N6CCC(Nc7ncc8ccc(=O)n(C9CCCC9)c8n7)CC6)c5)CC4)cccc3C2=O)C(=O)N1]

### Step 1: methyl 3-(2-hydroxyethoxy)-2-methylbenzoate

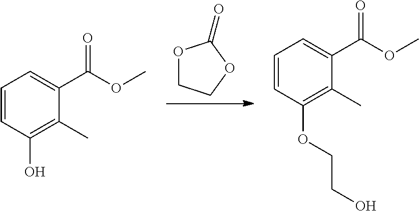

To a stirred solution of methyl 3-hydroxy-2-methylbenzoate (2.50 g, 15.04 mmol, 1.00 eq.) and 1,3-dioxolan-2-one (1.98 g, 22.48 mmol, 1.50 eq.) in DMF (30.0 mL) was added K2C03 (2.07 g, 14.98 mmol, 1.00 eq.). The resulting mixture was stirred at 120° C. under N₂ for 2 h. The reaction mixture was cooled, diluted with water and then extracted with EtOAc. The organic layer was washed water, brine, dried over Na₂SO₄, filtered, and then concentrated. The residue was purified by silica gel chromatography (EA:PE=1:4) to give the title compound (3.00 g, 94.9%) as a white solid.

### Step 2: methyl 2-(bromomethyl)-3-(2-hydroxyethoxy)benzoate

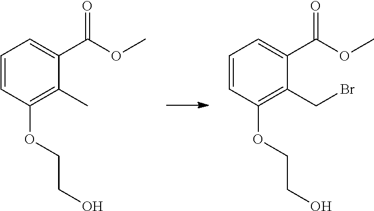 [SMILES: COC(=O)c1cccc(OCCO)c1C ; COC(=O)c1cccc(OCCO)c1CBr]

To a stirred solution of methyl 3-(2-hydroxyethoxy)-2-methylbenzoate (1.50 g, 7.14 mmol, 1.00 eq.) in CCl₄ (45.0 mL) was added NBS (1.46 g, 8.20 mmol, 1.15 eq.) and AIBN (117 mg, 0.71 mmol, 0.10 eq.). The resulting mixture was stirred under N₂ at 75° C. for 3 h, cooled and then concentrated. The residue was purified by silica gel chromatography (EA PE=1:3) to give the title compound (1.71 g, 82.9%) as a white solid.

### Step 3: 3-(4-(2-hydroxyethoxy)-1-oxoisoindolin-2-yl)piperidine-2,6-dione

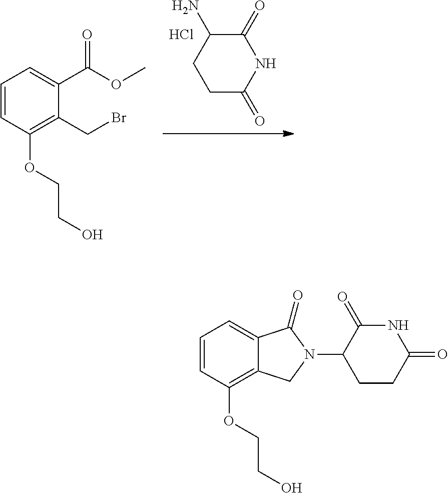 [SMILES: COC(=O)c1cccc(OCCO)c1CBr ; Cl ; NC1CCC(=O)NC1=O ; O=C1CCC(N2Cc3c(OCCO)cccc3C2=O)C(=O)N1]

To a stirred solution of methyl 2-(bromomethyl)-3-(2-hydroxyethoxy)benzoate (2.00 g, 6.92 mmol, 1.00 eq.) in ACN (70.0 mL) was added 3-aminopiperidine-2,6-dione hydrochloride (1.48 g, 8.99 mmol, 1.30 eq.) and TEA (1.04 g, 10.28 mmol, 1.49 eq.). The resulting mixture was stirred under N₂ at 80° C. overnight, cooled and then concentrated. The residue was purified by silica gel chromatography (DCM:MeOH=20:1) to give the title compound (2.00 g, 94.9%) as a blue solid.

### Step 4: 2-((2-(2,6-dioxopiperidin-3-yl)-1-oxoisoindolin-4-yl)oxy)ethyl 4-methylbenzenesulfonate

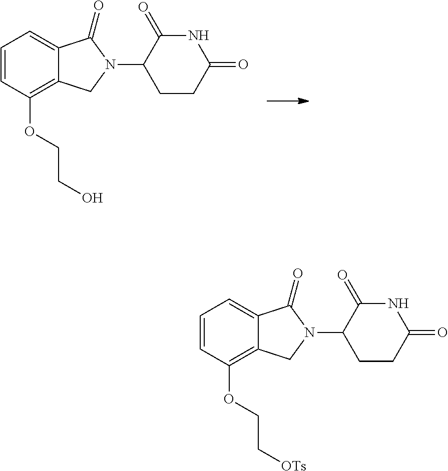 [SMILES: O=C1CCC(N2Cc3c(OCCO)cccc3C2=O)C(=O)N1 ; Cc1ccc(S(=O)(=O)OCCOc2cccc3c2CN(C2CCC(=O)NC2=O)C3=O)cc1]

To a stirred solution of 3-(4-(2-hydroxyethoxy)-1-oxoisoindolin-2-yl)piperidine-2,6-dione (500 mg, 1.64 mmol, 1.00 eq.) in DCM (10.0 mL) was added TEA (333 mg, 3.29 mmol, 2.00 eq.), TsCl (377 mg, 1.98 mmol, 1.21 eq.) and DMAP (20 mg, 0.16 mmol, 0.10 eq.) at 0° C. The resulting mixture was stirred at RT overnight, diluted with DCM, washed with water, brine, dried over Na₂SO₄, filtered, and then concentrated. The residue was purified by silica gel chromatography (DCM:MeOH=30:1) to give the title compound (200 mg, 26.8%) as a green solid.

### Step 5: benzyl 4-((methylsulfonyl)oxy)piperidine-1-carboxylate

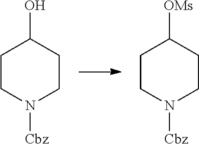 [SMILES: O=C(OCc1ccccc1)N1CCC(O)CC1 ; CS(=O)(=O)OC1CCN(C(=O)OCc2ccccc2)CC1]

To a stirred solution of benzyl 4-hydroxypiperidine-1-carboxylate (2.00 g, 8.50 mmol, 1.00 eq.) in DCM (20.0 mL) was added TEA (2.57 g, 25.40 mmol, 3.00 eq.) and MsCl (1.16 g, 10.13 mmol, 1.20 eq.) at 0° C. The resulting mixture was stirred at RT overnight, diluted with water and then extracted with DCM. The organic layer was washed with water, brine, dried over Na₂SO₄, filtered, and concentrated to give the crude title compound (2.60 g, 97.6%) as a yellow oil, which was used for next step without further purification.

### Step 6: tert-butyl (1-((3-(piperidin-4-yloxy)phenyl)sulfonyl)piperidin-4-yl)carbamate

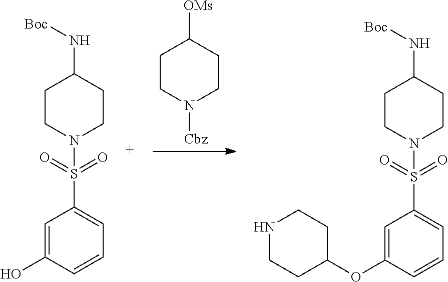 [SMILES: CC(C)(C)OC(=O)NC1CCN(S(=O)(=O)c2cccc(O)c2)CC1 ; CS(=O)(=O)OC1CCN(C(=O)OCc2ccccc2)CC1 ; CC(C)(C)OC(=O)NC1CCN(S(=O)(=O)c2cccc(OC3CCNCC3)c2)CC1]

Benzyl 4-((methylsulfonyl)oxy)piperidine-1-carboxylate was converted to the title compound by proceeding analogously as described in Example 12, Steps 5-6 above.

### Step 7: tert-butyl (1-((3-((1-(2-((2-(2,6-dioxopiperidin-3-yl)-1-oxoisoindolin-4-yl)oxy)ethyl)piperidin-4-yl)oxy)phenyl)sulfonyl)piperidin-4-yl)carbamate

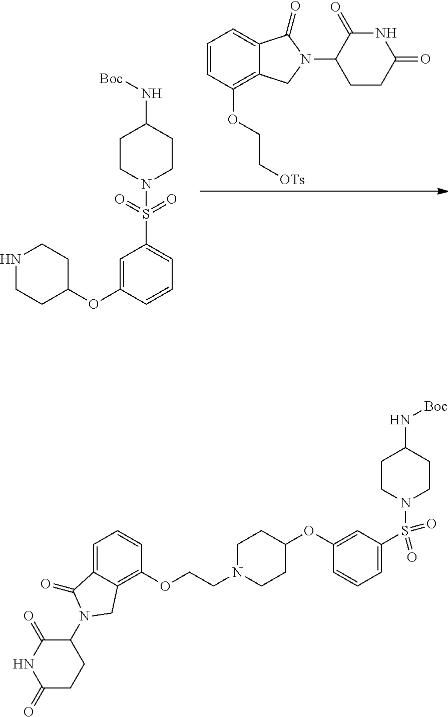 [SMILES: CC(C)(C)OC(=O)NC1CCN(S(=O)(=O)c2cccc(OC3CCNCC3)c2)CC1 ; Cc1ccc(S(=O)(=O)OCCOc2cccc3c2CN(C2CCC(=O)NC2=O)C3=O)cc1 ; CC(C)(C)OC(=O)NC1CCN(S(=O)(=O)c2cccc(OC3CCN(CCOc4cccc5c4CN(C4CCC(=O)NC4=O)C5=O)CC3)c2)CC1]

To a stirred solution of 2-((2-(2,6-dioxopiperidin-3-yl)-1-oxoisoindolin-4-yl)oxy)ethyl 4-methylbenzenesulfonate (50 mg, 0.11 mmol, 1.10 eq.) and tert-butyl (1-((3-(piperidin-4-yloxy)phenyl)sulfonyl)piperidin-4-yl)carbamate (44 mg, 0.10 mmol, 1.00 eq.) in ACN (2.0 mL) was added KI (15 mg, 0.090 mmol, 0.90 eq.) and DIPEA (35 mg, 0.27 mmol, 2.70 eq.) at RT under N₂. The resulting mixture was stirred at 100° C. under microwave for 3 h. The reaction mixture was cooled and concentrated, and then purified by silica gel chromatography (DCM:MeOH=20:1) to give the title compound (60 mg, 82.7%) as a yellow oil.

### Step 8: 3-(4-(2-(4-(3-((4-((8-cyclopentyl-7-oxo-7,8-dihydropyrido[2,3-d]pyrimidin-2-yl)-amino)piperidin-1-yl)sulfonyl)phenoxy)piperidin-1-yl)ethoxy)-1-oxoisoindolin-2-yl)piperidine-2,6-dione

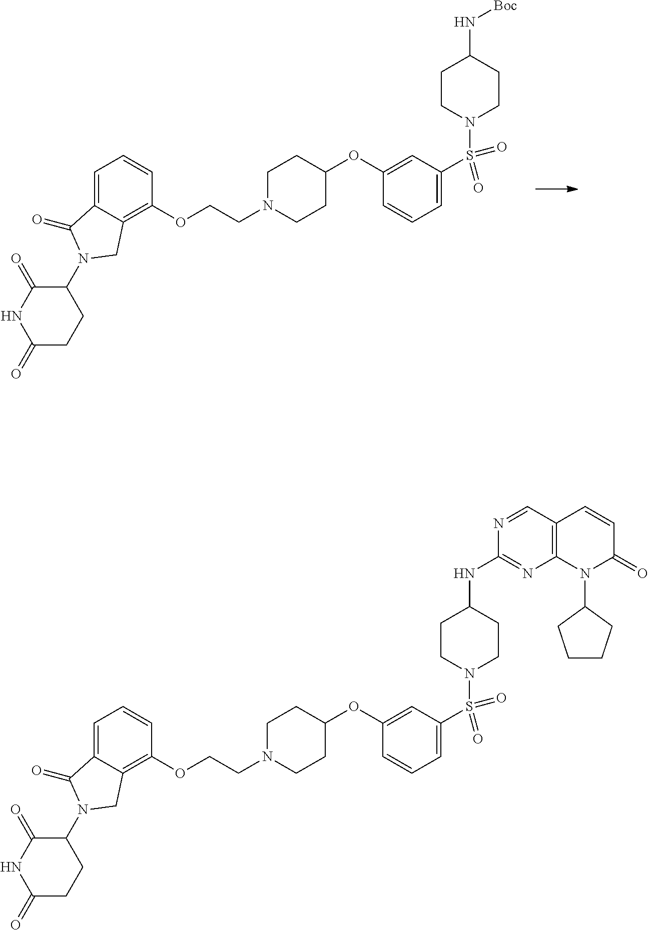 [SMILES: CC(C)(C)OC(=O)NC1CCN(S(=O)(=O)c2cccc(OC3CCN(CCOc4cccc5c4CN(C4CCC(=O)NC4=O)C5=O)CC3)c2)CC1 ; O=C1CCC(N2Cc3c(OCCN4CCC(Oc5cccc(S(=O)(=O)N6CCC(Nc7ncc8ccc(=O)n(C9CCCC9)c8n7)CC6)c5)CC4)cccc3C2=O)C(=O)N1]

tert-Butyl (1-((3-((1-(2-((2-(2,6-dioxopiperidin-3-yl)-1-oxoisoindolin-4-yl)oxy)-ethyl)piperidin-4-yl)oxy)phenyl)sulfonyl)piperidin-4-yl)carbamate was converted to the title compound by proceeding analogously as described in Example 10, Steps 7-8 above. MS (ES, m/z): [M+1]⁺=839.4.

### Example 30

### Synthesis of 3-(4-(2-(3-(3-((4-((8-cyclopentyl-7-oxo-7,8-dihydropyrido[2,3-d]pyrimidin-2-yl)amino)piperidin-1-yl)sulfonyl)phenoxy)azetidin-1-yl)ethoxy)-1-oxoisoindolin-2-yl)piperidine-2,6-dione

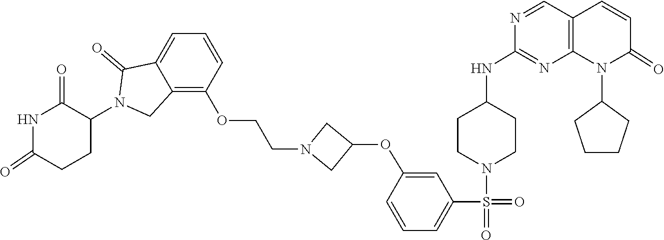 [SMILES: O=C1CCC(N2Cc3c(OCCN4CC(Oc5cccc(S(=O)(=O)N6CCC(Nc7ncc8ccc(=O)n(C9CCCC9)c8n7)CC6)c5)C4)cccc3C2=O)C(=O)N1]

### Step 1: tert-butyl (1-((3-((1-(2-((2-(2,6-dioxopiperidin-3-yl)-1-oxoisoindolin-4-yl)oxy)ethyl)azetidin-3-yl)oxy)phenyl)sulfonyl)piperidin-4-yl)carbamate

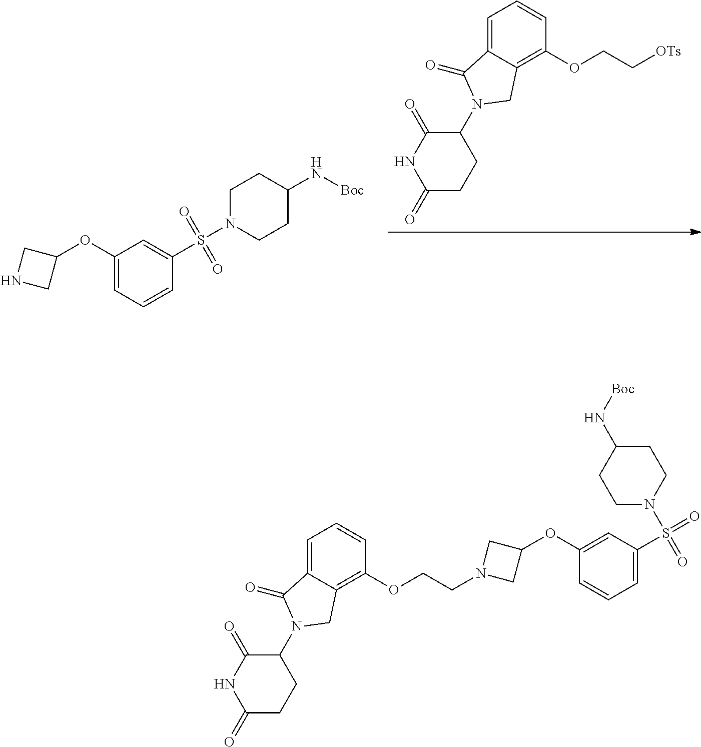 [SMILES: CC(C)(C)OC(=O)NC1CCN(S(=O)(=O)c2cccc(OC3CNC3)c2)CC1 ; Cc1ccc(S(=O)(=O)OCCOc2cccc3c2CN(C2CCC(=O)NC2=O)C3=O)cc1 ; CC(C)(C)OC(=O)NC1CCN(S(=O)(=O)c2cccc(OC3CN(CCOc4cccc5c4CN(C4CCC(=O)NC4=O)C5=O)C3)c2)CC1]

tert-Butyl (1-((3-(azetidin-3-yloxy)phenyl)sulfonyl)piperidin-4-yl)carbamate was converted to the title compound by proceeding analogously as described in Example 29, Step 7, which was then converted to 3-(4-(2-(3-(3-((4-((8-cyclopentyl-7-oxo-7,8-dihydropyrido[2,3-d]-pyrimidin-2-yl)amino)piperidin-1-yl)sulfonyl)phenoxy)azetidin-1-yl)ethoxy)-1-oxoisoindolin-2-yl)piperidine-2,6-dione as described in Example 10, Steps 7-8 above. MS (ES, m/z): [M+1]⁺=811.3.

Proceeding analogously as described in Example 23, the following compounds were prepared.

| | | | |
|---|---|---|---|
| Ex- am- ple 31 | 3-(4- (1′-((4- ((8- cyclopentyl- 7-oxo-7,8- dihydro- pyrido [2,3-d] pyrimidin-2- yl)amino) piperidin-1- | 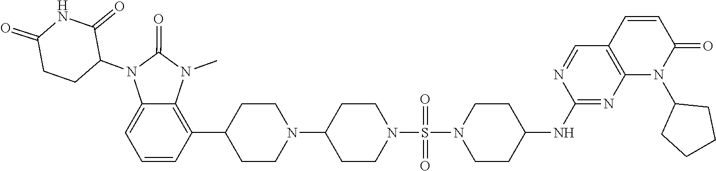 [SMILES: Cn1c(=O)n(C2CCC(=O)NC2=O)c2cccc(C3CCN(C4CCN(S(=O)(=O)N5CCC(Nc6ncc7ccc(=O)n(C8CCCC8)c7n6)CC5)CC4)CC3)c21] | [M + 1]⁺ = 801.3 |
| | yl)sulfonyl)- | | |
| | [1,4′- | | |
| | bipiperidin]- | | |
| | 4-yl)-3- | | |
| | methyl-2- | | |
| | oxo-2,3- | | |
| | dihydro- | | |
| | 1H- | | |
| | benzo[d] | | |
| | imidazol-1- | | |
| | yl) | | |
| | piperidine- | | |
| | 2,6-dione | | |
| Ex- am- ple 32 | 3-(4-(1- (2-((4-((8- cyclopentyl- 7-oxo-7,8- dihydro- pyrido [2,3-d] pyrimidin-2- yl)amino) piperidin-1- yl) | 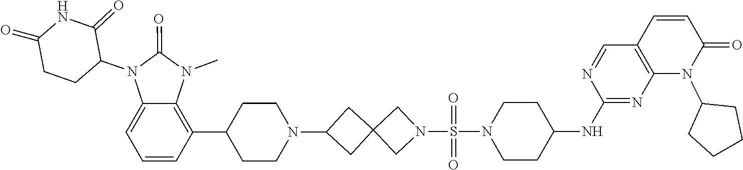 [SMILES: Cn1c(=O)n(C2CCC(=O)NC2=O)c2cccc(C3CCN(C4CC5(C4)CN(S(=O)(=O)N4CCC(Nc6ncc7ccc(=O)n(C8CCCC8)c7n6)CC4)C5)CC3)c21] | [M + 1]⁺ = 813.4 |
| | sulfonyl)-2- | | |
| | azaspiro[3.3] | | |
| | heptan-6- | | |
| | yl)piperidin- | | |
| | 4-yl)-3- | | |
| | methyl-2- | | |
| | oxo-2,3- | | |
| | dihydro- | | |
| | 1H- | | |
| | benzo[d] | | |
| | imidazol-1- | | |
| | yl) | | |
| | piperidine- | | |
| | 2,6-dione | | |

### Example 33

### Synthesis of 3-(5-(4-(3-((4-((8-cyclopentyl-7-oxo-7,8-dihydropyrido[2,3-d]pyrimidin-2-yl)amino)piperidin-1-yl)sulfonyl)phenoxy)piperidin-1-yl)-1-oxoisoindolin-2-yl)piperidine-2,6-dione

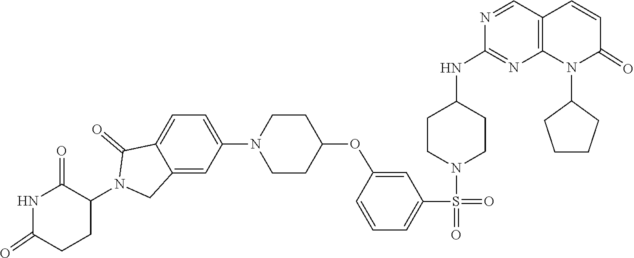 [SMILES: O=C1CCC(N2Cc3cc(N4CCC(Oc5cccc(S(=O)(=O)N6CCC(Nc7ncc8ccc(=O)n(C9CCCC9)c8n7)CC6)c5)CC4)ccc3C2=O)C(=O)N1]

### Step 1: tert-butyl (1-((3-((1-(2-(2,6-dioxopiperidin-3-yl)-1-oxoisoindolin-5-yl)piperidin-4-yl)oxy)phenyl)sulfonyl)piperidin-4-yl)carbamate

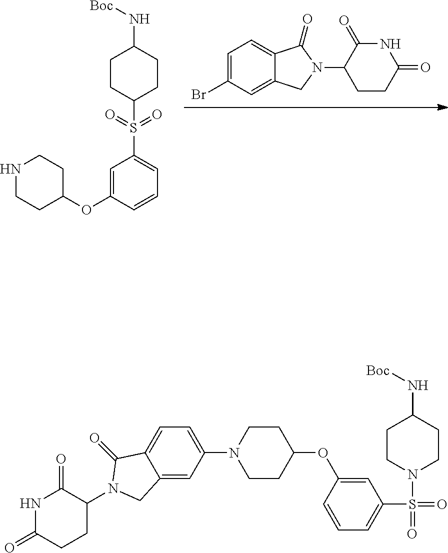 [SMILES: CC(C)(C)OC(=O)NC1CCC(S(=O)(=O)c2cccc(OC3CCNCC3)c2)CC1 ; O=C1CCC(N2Cc3cc(Br)ccc3C2=O)C(=O)N1 ; CC(C)(C)OC(=O)NC1CCN(S(=O)(=O)c2cccc(OC3CCN(c4ccc5c(c4)CN(C4CCC(=O)NC4=O)C5=O)CC3)c2)CC1]

To a stirred solution of tert-butyl (1-((3-(piperidin-4-yloxy)phenyl)sulfonyl)piperidin-4-yl) carbamate (300 mg, 0.93 mmol, 1.00 eq.) and 3-(5-bromo-1-oxoisoindolin-2-yl)piperidine-2,6-dione (448 mg, 1.02 mmol, 1.10 eq.) in 1,4-dioxane (10.0 mL) was added Cs₂CO₃ (603 mg, 1.86 mmol, 2.00 eq.), Pd(OAc)₂ (41 mg, 0.19 mmol, 0.20 eq.) and X-Phos (176 mg, 0.37 mmol, 0.40 eq.) and the resulting mixture was stirred at 105° C. under N2 for 2 days. The reaction mixture was extracted with DCM and water. The organic layer was washed with brine and dried over Na₂SO₄ and concentrated. Purification by flash chromatography gave title compound (300 mg, crude) as a yellow solid.

### Step 2: 3-(5-(4-(3-((4-((8-cyclopentyl-7-oxo-7,8-dihydropyrido[2,3-d]pyrimidin-2-yl)-amino)piperidin-1-yl)sulfonyl)phenoxy)piperidin-1-yl)-1-oxoisoindolin-2-yl)piperidine-2,6-dione

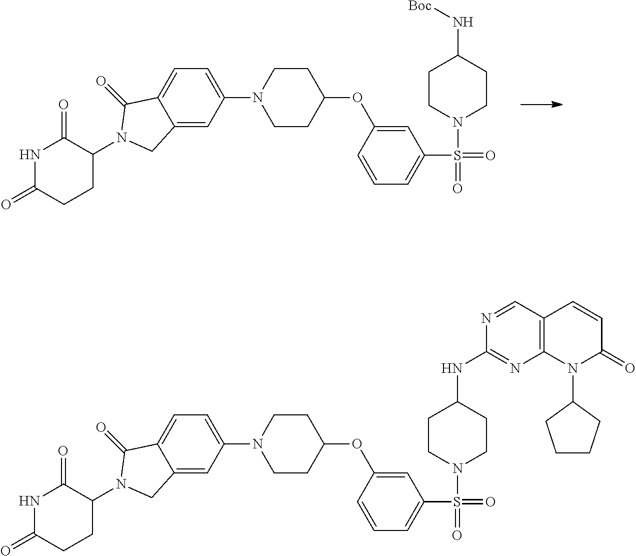 [SMILES: CC(C)(C)OC(=O)NC1CCN(S(=O)(=O)c2cccc(OC3CCN(c4ccc5c(c4)CN(C4CCC(=O)NC4=O)C5=O)CC3)c2)CC1 ; O=C1CCC(N2Cc3cc(N4CCC(Oc5cccc(S(=O)(=O)N6CCC(Nc7ncc8ccc(=O)n(C9CCCC9)c8n7)CC6)c5)CC4)ccc3C2=O)C(=O)N1]

tert-butyl (1-((3-((1-(2-(2,6-Dioxopiperidin-3-yl)-1-oxoisoindolin-5-yl)piperidin-4-yl)-oxy)phenyl)sulfonyl)piperidin-4-yl)carbamate was converted to the title compound by proceeding analogously as described in Example 7, Steps 5-6. Purification of the crude product by prep-HPLC gave title compound (6 mg, 5%) as a white solid. MS (ES, m/z): [M+1]⁺=795.5

### Example 34

### Synthesis of 3-(5-(4-((4-(3-((4-((8-cyclopentyl-7-oxo-7,8-dihydropyrido[2,3-d]pyrimidin-2-yl)amino)piperidin-1-yl)sulfonyl)phenyl)piperidin-1-yl)methyl)piperidin-1-yl)-1-oxoisoindolin-2-yl)piperidine-2,6-dione

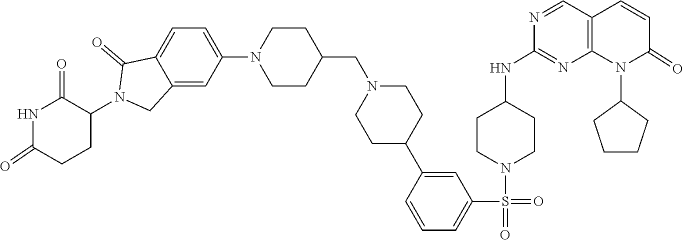 [SMILES: O=C1CCC(N2Cc3cc(N4CCC(CN5CCC(c6cccc(S(=O)(=O)N7CCC(Nc8ncc9ccc(=O)n(C%10CCCC%10)c9n8)CC7)c6)CC5)CC4)ccc3C2=O)C(=O)N1]

### Step 1: benzyl 4-(3-((4-((tert-butoxycarbonyl)amino)piperidin-1-yl)sulfonyl)phenyl)-5,6-dihydropyridine-1 (2H)-carboxylate

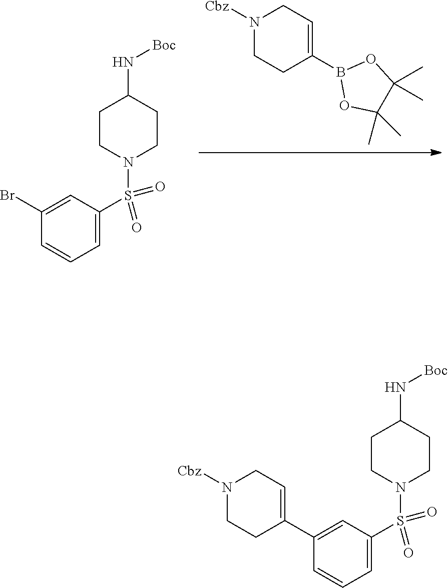 [SMILES: CC(C)(C)OC(=O)NC1CCN(S(=O)(=O)c2cccc(Br)c2)CC1 ; CC1(C)OB(C2=CCN(C(=O)OCc3ccccc3)CC2)OC1(C)C ; CC(C)(C)OC(=O)NC1CCN(S(=O)(=O)c2cccc(C3=CCN(C(=O)OCc4ccccc4)CC3)c2)CC1]

A mixture of tert-butyl (1-((3-bromophenyl)sulfonyl)piperidin-4-yl)carbamate (3.00 g, 7.18 mmol, 1.00 eq.), benzyl 4-(4,4,5,5-tetramethyl-1,3,2-dioxaborolan-2-yl)-5,6-dihydropyridine-1 (2H)-carboxylate (3.20 g, 9.33 mmol, 1.30 eq.), X-phos-G3 (608.0 mg, 0.72 mmol, 0.10 eq.) and K₃PO₄ (4.57 g, 21.54 mmol, 3.00 eq.) in 1,4-dioxane (70.0 mL) and H₂O (7.0 mL) was stirred at 60° C. under N2 for 6 h. The resulting mixture was concentrated and the residue was purified by silica gel column chromatography, eluted with PE/EtOAc (4:1), to afford the title compound (4.0 g, 100%) as a yellow solid.

### Step 2: tert-butyl (1-((3-(piperidin-4-yl)phenyl)sulfonyl)piperidin-4-yl)carbamate

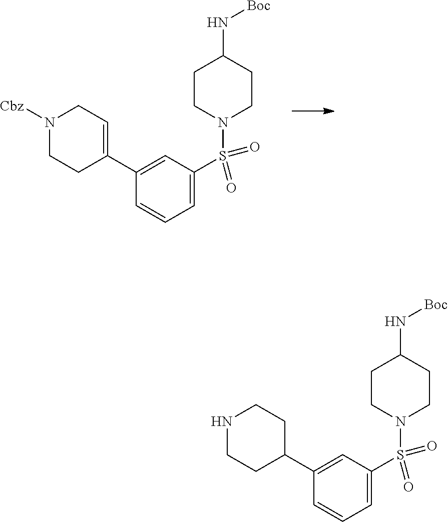 [SMILES: CC(C)(C)OC(=O)NC1CCN(S(=O)(=O)c2cccc(C3=CCN(C(=O)OCc4ccccc4)CC3)c2)CC1 ; CC(C)(C)OC(=O)NC1CCN(S(=O)(=O)c2cccc(C3CCNCC3)c2)CC1]

A mixture of benzyl 4-(3-((4-((tert-butoxycarbonyl)amino)piperidin-1-yl)sulfonyl)phenyl)-5,6-dihydropyridine-1 (2H)-carboxylate (4.00 g, 7.20 mmol, 1.00 eq.) and Pd/C (800 mg) in MeOH (40.0 mL) was stirred at 50° C. under H2 (50 psi) for 16 h. The mixture was filtered and concentrated to afford the title compound (3.00 g, 100%) as a white solid.

### Step 3: benzyl 4-((4-(3-((4-((tert-butoxycarbonyl)amino)piperidin-1-yl)sulfonyl)phenyl)piperidin-1-yl)methyl)piperidine-1-carboxylate

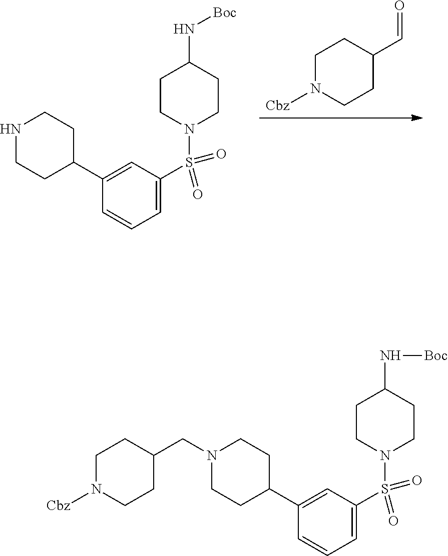 [SMILES: CC(C)(C)OC(=O)NC1CCN(S(=O)(=O)c2cccc(C3CCNCC3)c2)CC1 ; O=CC1CCN(C(=O)OCc2ccccc2)CC1 ; CC(C)(C)OC(=O)NC1CCN(S(=O)(=O)c2cccc(C3CCN(CC4CCN(C(=O)OCc5ccccc5)CC4)CC3)c2)CC1]

To a solution of tert-butyl (1-((3-(piperidin-4-yl)phenyl)sulfonyl)piperidin-4-yl)carbamate (3.00 g, 7.10 mmol, 1.00 eq.) in DCE (20.0 mL) and MeOH (20.0 mL) was added benzyl 4-formylpiperidine-1-carboxylate (2.63 g, 10.65 mmol, 1.50 eq.) and AcOH (426.0 mg, 7.10 mmol, 1.00 eq.) and the solution was stirred at RT for 1 h. NaBH₃CN (1.34 g, 21.30 mmol, 3.00 eq.) was added and the mixture was stirred at RT for 3 h. The resulting mixture was concentrated and the residue was purified by silica gel column chromatography, eluted with DCM/MeOH (60:1), to afford the title compound (3.80 g, 81.9%) as a white solid.

### Step 4: tert-butyl (1-((3-(1-(piperidin-4-ylmethyl)piperidin-4-yl)phenyl)sulfonyl)piperidin-4-yl)-carbamate

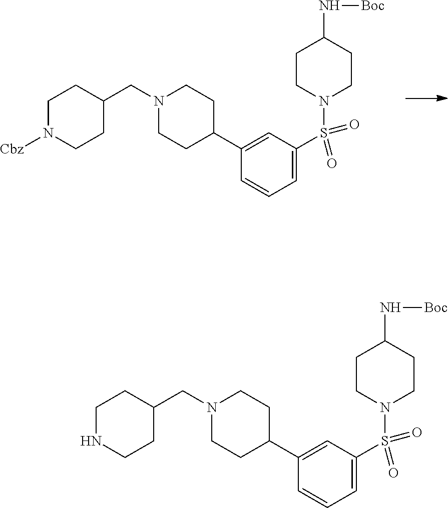 [SMILES: CC(C)(C)OC(=O)NC1CCN(S(=O)(=O)c2cccc(C3CCN(CC4CCN(C(=O)OCc5ccccc5)CC4)CC3)c2)CC1 ; CC(C)(C)OC(=O)NC1CCN(S(=O)(=O)c2cccc(C3CCN(CC4CCNCC4)CC3)c2)CC1]

A mixture of benzyl 4-((4-(3-((4-((tert-butoxycarbonyl)amino)piperidin-1-yl)-sulfonyl)phenyl)piperidin-1-yl)methyl)piperidine-1-carboxylate (3.80 g, 5.81 mmol, 1.00 eq.) and Pd/C (800 mg) in MeOH (40.0 mL) was stirred at 50° C. under H2 (50 psi) for 16 h. The mixture was filtered and concentrated to afford the title compound (2.80 g, 93.3%) as a white solid.

### Step 5: methyl 4-(4-((4-(3-((4-((tert-butoxycarbonyl)amino)piperidin-1-yl)sulfonyl)-phenyl)piperidin-1-yl)methyl)piperidin-1-yl)-2-cyanobenzoate

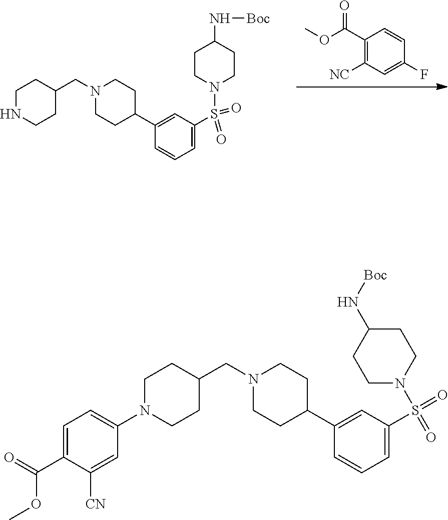 [SMILES: CC(C)(C)OC(=O)NC1CCN(S(=O)(=O)c2cccc(C3CCN(CC4CCNCC4)CC3)c2)CC1 ; COC(=O)c1ccc(F)cc1C#N ; COC(=O)c1ccc(N2CCC(CN3CCC(c4cccc(S(=O)(=O)N5CCC(NC(=O)OC(C)(C)C)CC5)c4)CC3)CC2)cc1C#N]

A solution of tert-butyl (1-((3-(1-(piperidin-4-ylmethyl)piperidin-4-yl)phenyl)-sulfonyl)piperidin-4-yl)carbamate (2.80 g, 5.40 mmol, 1.00 eq.), methyl 2-cyano-4-fluorobenzoate (1.06 g, 5.94 mmol, 1.10 eq.) and DIEA (2.09 g, 16.20 mmol, 3.00 eq.) in DMSO (30.0 mL) was stirred at 120° C. under N2 for 16 h. The mixture was cooled to RT, diluted with water, and then extracted with EtOAc. The combined organic layer was washed with water, dried over anhydrous Na₂SO₄, filtered, and then concentrated. The residue was purified by silica gel column chromatography, eluted with DCM/MeOH (100:1), to afford the title compound (2.8 g, 76.5%) as a brown solid.

### Step 6: methyl 4-(4-((4-(3-((4-((tert-butoxycarbonyl)amino)piperidin-1-yl)sulfonyl)-phenyl)piperidin-1-yl)methyl)piperidin-1-yl)-2-formylbenzoate

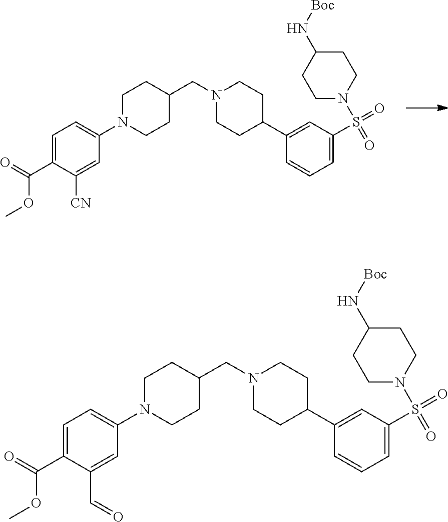 [SMILES: COC(=O)c1ccc(N2CCC(CN3CCC(c4cccc(S(=O)(=O)N5CCC(NC(=O)OC(C)(C)C)CC5)c4)CC3)CC2)cc1C#N ; COC(=O)c1ccc(N2CCC(CN3CCC(c4cccc(S(=O)(=O)N5CCC(NC(=O)OC(C)(C)C)CC5)c4)CC3)CC2)cc1C=O]

A mixture of methyl 4-(4-((4-(3-((4-((tert-butoxycarbonyl)amino)piperidin-1-yl)-sulfonyl)phenyl)piperidin-1-yl)methyl)piperidin-1-yl)-2-cyanobenzoate (1.01 g, 1.50 mmol, 1.00 eq.), NaH₂PO₂·H₂O (1.59 g, 15.00 mmol, 10.00 eq.) and Raney Ni (1.60 g) in pyridine (10.0 mL), H₂O (5.0 mL) and AcOH (5.0 mL) was stirred for 16 h at 70° C. under nitrogen atmosphere. The resulting mixture was diluted with EtOAc and washed with water, brine, dried over anhydrous Na₂SO₄ and concentrated. The residue was purified by silica gel column chromatography, eluted with DCM/MeOH (80:1), to afford the title compound (400 mg, 39.2%) as a light-yellow solid.

### Step 7: tert-butyl (1-((3-(1-((1-(2-(2,6-dioxopiperidin-3-yl)-1-oxoisoindolin-5-yl)piperidin-4-yl)methyl)piperidin-4-yl)phenyl)sulfonyl)piperidin-4-yl)carbamate

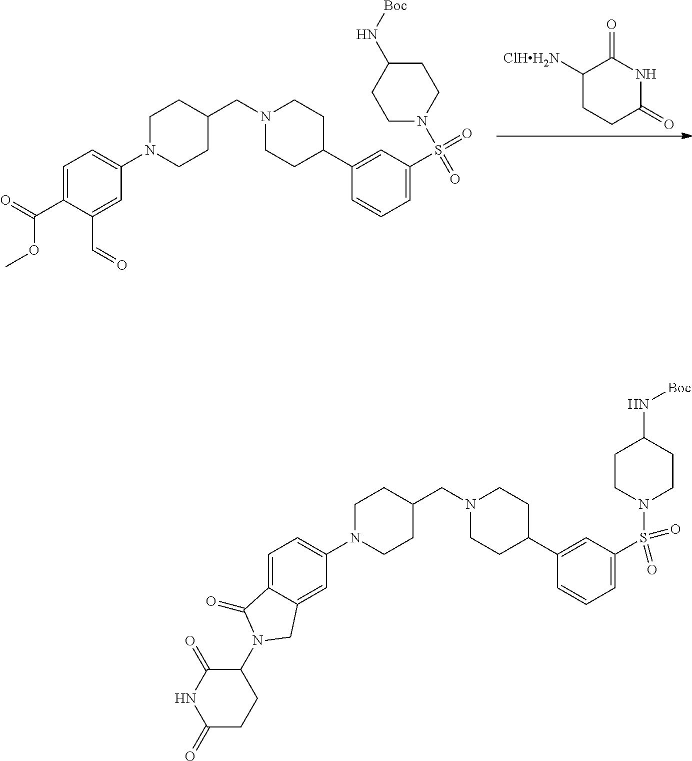

A mixture of 3-aminopiperidine-2,6-dione hydrochloride (126 mg, 0.77 mmol, 1.30 eq.) and DIEA (184 mg, 1.43 mmol, 2.40 eq.) in dry DCM (5.0 mL) was stirred at RT for 10 min. and then a solution of methyl 4-(4-((4-(3-((4-((tert-butoxycarbonyl)amino)piperidin-1-yl)sulfonyl)phenyl)piperidin-1-yl)methyl)piperidin-1-yl)-2-formylbenzoate (400 mg, 0.59 mmol, 1.00 eq.) in dry DCM (5.0 mL) and AcOH (134 mg, 2.23 mmol, 3.80 eq.) was added. The mixture was stirred at 45° C. under N2 for 3 h. The mixture was cooled to 0° C. and NaBH(OAc)3 (375 mg, 1.77 mmol, 3.00 eq.) was added to this mixture. The mixture was stirred at RT for 1 h and then at 45° C. under N₂ for 16 h. The mixture was cooled, diluted with water, and then extracted with DCM. The combined organic layer was washed with water, dried over anhydrous Na₂SO₄, filtered, and concentrated. The residue was purified by silica gel column chromatography, eluted with DCM/MeOH (40:1), to afford the title compound (260 mg, 57.7%) as a yellow solid.

### Step 8: 3-(5-(4-((4-(3-((4-((8-cyclopentyl-7-oxo-7,8-dihydropyrido[2,3-d]pyrimidin-2-yl)-amino)piperidin-1-yl)sulfonyl)phenyl)piperidin-1-yl)methyl)piperidin-1-yl)-1-oxoisoindolin-2-yl)piperidine-2,6-dione

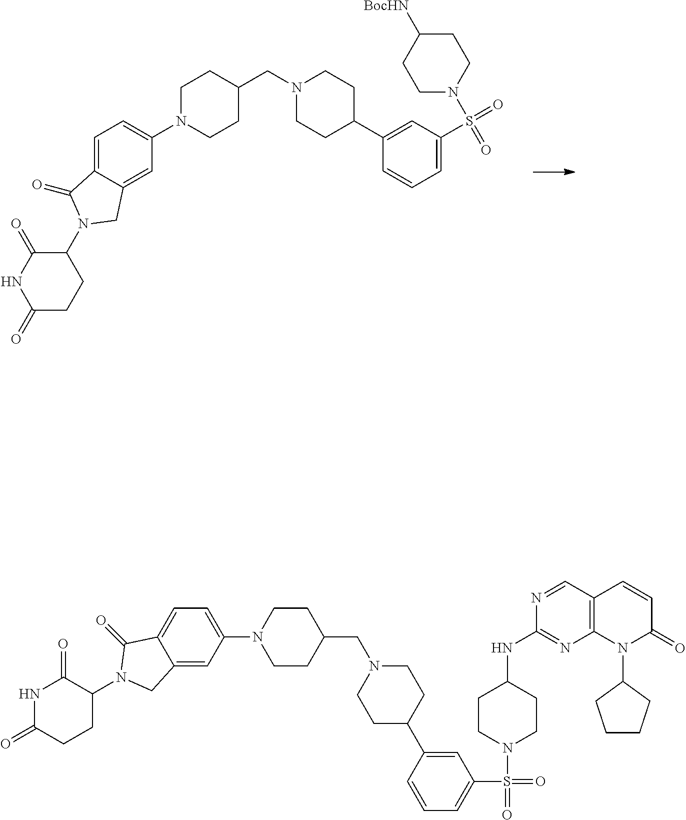 [SMILES: CC(C)(C)OC(=O)NC1CCN(S(=O)(=O)c2cccc(C3CCN(CC4CCN(c5ccc6c(c5)CN(C5CCC(=O)NC5=O)C6=O)CC4)CC3)c2)CC1 ; O=C1CCC(N2Cc3cc(N4CCC(CN5CCC(c6cccc(S(=O)(=O)N7CCC(Nc8ncc9ccc(=O)n(C%10CCCC%10)c9n8)CC7)c6)CC5)CC4)ccc3C2=O)C(=O)N1]

tert-butyl (1-((3-(1-((1-(2-(2,6-Dioxopiperidin-3-yl)-1-oxoisoindolin-5-yl)piperidin-4-yl)methyl)piperidin-4-yl)phenyl)sulfonyl)piperidin-4-yl)carbamate was converted to the title compound by proceeding analogously as described in Example 7, Steps 5-6. The residue was purified by pre-HPLC and TLC, eluted with DCM/MeOH (15:1), to afford the title compound (85.0 mg, 28.5%) as a white solid. MS (ES, m/z): [M+1]⁺=876.4.

### Example 35

### Synthesis of 3-(5-(1-((1-(3-((4-((8-cyclopentyl-7-oxo-7,8-dihydropyrido[2,3-d]pyrimidin-2-yl)amino)piperidin-1-yl)sulfonyl)phenyl)piperidin-4-yl)methyl)piperidin-4-yl)-1-oxoisoindolin-2-yl)piperidine-2,6-dione

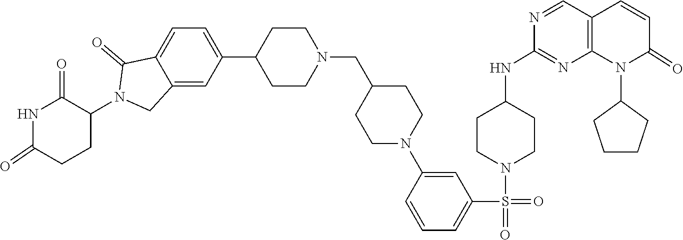 [SMILES: O=C1CCC(N2Cc3cc(C4CCN(CC5CCN(c6cccc(S(=O)(=O)N7CCC(Nc8ncc9ccc(=O)n(C%10CCCC%10)c9n8)CC7)c6)CC5)CC4)ccc3C2=O)C(=O)N1]

### Step 1: 3-5-bromo-1-oxoisoindolin-2-ylpiperidine-2,6-dione

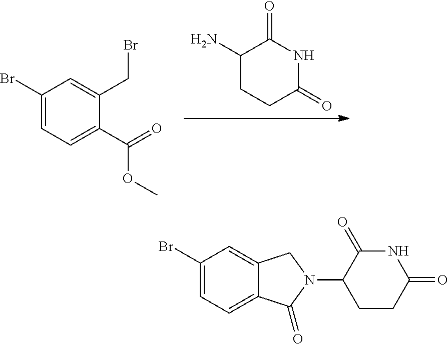 [SMILES: COC(=O)c1ccc(Br)cc1CBr ; NC1CCC(=O)NC1=O ; O=C1CCC(N2Cc3cc(Br)ccc3C2=O)C(=O)N1]

To a stirred solution of methyl 4-bromo-2-(bromomethyl)benzoate (20.00 g, 64.91 mmol, 1.00 eq.) and 3-aminopiperidine-2,6-dione (11.71 g, 71.41 mmol, 1.10 eq.) in DMF was added K2C03 (26.87 g, 194.71 mmol, 3.00 eq.). The resulting mixture was stirred at room 70° C. overnight under N2 atmosphere. The mixture was poured into water after the reaction was complete and the product was extracted. The crude product was purified by flash column (PE:EA=2:1) to give the title compound (10.37 g, 49.62%) as a white solid.

### Step 2: tert-butyl 4-(2-(2,6-dioxopiperidin-3-yl)-1-oxoisoindolin-5-yl)-5,6-dihydropyridine-1 (2H)-carboxylate

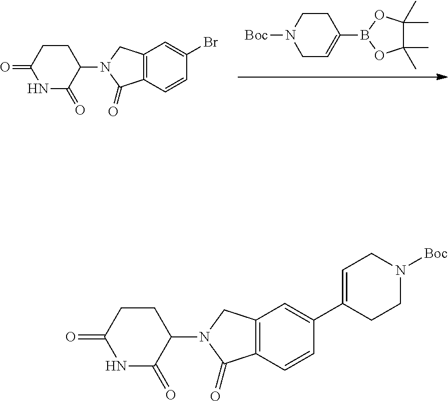 [SMILES: O=C1CCC(N2Cc3cc(Br)ccc3C2=O)C(=O)N1 ; CC(C)(C)OC(=O)N1CC=C(B2OC(C)(C)C(C)(C)O2)CC1 ; CC(C)(C)OC(=O)N1CC=C(c2ccc3c(c2)CN(C2CCC(=O)NC2=O)C3=O)CC1]

To a stirred solution of 3-(5-bromo-1-oxoisoindolin-2-yl)piperidine-2,6-dione (1.00 g, 3.11 mmol, 1.00 eq.) in DMF (10.0 mL) was added tert-butyl 4-(4,4,5,5-tetramethyl-1,3,2-dioxaborolan-2-yl)-5,6-dihydropyridine-1 (2H)-carboxylate (1.25 g, 4.04 mmol, 1.30 eq.), K₃PO₄ (800 mg, 3.73 mmol, 1.20 eq) and Pd(dppf)Cl₂ (114 mg, 0.16 mmol, 0.05 eq) at room temperature. The resulting mixture was stirred at 90° C. for 12 h, then concentrated and purified by silica gel column chromatography eluting with PE/EA (1:2) to give title compound (420 mg, 30%) as yellow solid.

### Step 3: tert-butyl 4-(2-(2,6-dioxopiperidin-3-yl)-1-oxoisoindolin-5-yl)piperidine-1-carboxylate

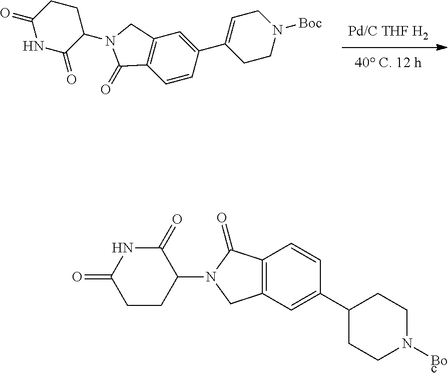 [SMILES: CC(C)(C)OC(=O)N1CC=C(c2ccc3c(c2)CN(C2CCC(=O)NC2=O)C3=O)CC1 ; CC(C)(C)OC(=O)N1CCC(c2ccc3c(c2)CN(C2CCC(=O)NC2=O)C3=O)CC1]

To a stirred solution of tert-butyl 4-(2-(2,6-dioxopiperidin-3-yl)-1-oxoisoindolin-5-yl)-5,6-dihydropyridine-1 (2H)-carboxylate (200 mg, 0.47 mmol, 1.00 eq.) in THF (2.0 mL) was added Pd/C (40 mg, 20% w/w). The resulting mixture was stirred at 40° C. for 12 h under H₂, filtered and concentrated to give the title compound (180 mg, 89.6%) as white solid.

### Step 4: 3-(1-oxo-5-(piperidin-4-yl)isoindolin-2-yl)piperidine-2,6-dione

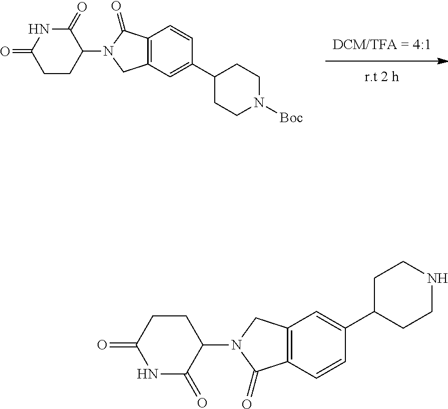 [SMILES: CC(C)(C)OC(=O)N1CCC(c2ccc3c(c2)CN(C2CCC(=O)NC2=O)C3=O)CC1 ; O=C1CCC(N2Cc3cc(C4CCNCC4)ccc3C2=O)C(=O)N1]

To a stirred solution of tert-butyl 4-(2-(2,6-dioxopiperidin-3-yl)-1-oxoisoindolin-5-yl)piperidine-1-carboxylate (100 mg, 0.234 mmol, 1.00 eq.) was in DCM/TFA=4:1 (2.5 mL). The reaction mixture was stirred at RT for 2 h and then concentrated to give the title compound (76.6 mg, crude) as brown solid.

### Step 5: tert-butyl (1-((3-(4-((4-(2-(2,6-dioxopiperidin-3-yl)-1-oxoisoindolin-5-yl)piperidin-1-yl)methyl)piperidin-1-yl)phenyl)sulfonyl)piperidin-4-yl)carbamate

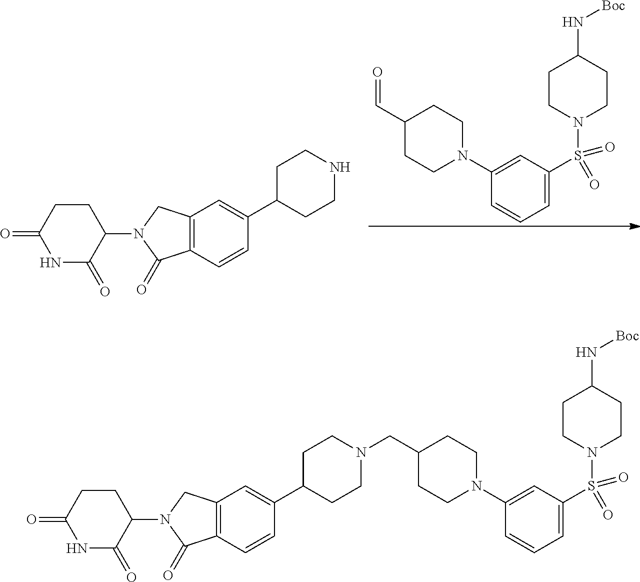 [SMILES: O=C1CCC(N2Cc3cc(C4CCNCC4)ccc3C2=O)C(=O)N1 ; CC(C)(C)OC(=O)NC1CCN(S(=O)(=O)c2cccc(N3CCC(C=O)CC3)c2)CC1 ; CC(C)(C)OC(=O)NC1CCN(S(=O)(=O)c2cccc(N3CCC(CN4CCC(c5ccc6c(c5)CN(C5CCC(=O)NC5=O)C6=O)CC4)CC3)c2)CC1]

To a stirred solution of 3-(1-oxo-5-(piperidin-4-yl)isoindolin-2-yl)piperidine-2,6-dione (76.60 mg, 0.23 mmol, 1.00 eq) in THF (1.0 mL) was added DMF (1.0 mL), HCOOH (1 drop) and tert-butyl (1-((3-(4-formylpiperidin-1-yl)phenyl)sulfonyl)piperidin-4-yl)carbamate (105.60 mg, 0.23 mmol 1.00 eq) and the resulting mixture was stirred at 45° C. for 0.5 h. NaBH₃CN (29.40 mg, 0.47 mmol, 2.00 eq) was added at RT and the reaction mixture was stirred at RT for 12 h. The mixture was diluted with water and extracted with EtOAc. The combined organic layer was washed with water and brine. The organic layer was concentrated and the residue was purified by silica gel column chromatography, eluted with DCM:MeOH (0-100%), to give compound (80 mg, 44.8%) as a white solid.

### Step 6: 3-(5-(1-((1-(3-((4-((8-cyclopentyl-7-oxo-7,8-dihydropyrido[2,3-d]pyrimidin-2-yl)amino)-piperidin-1-yl)sulfonyl)phenyl)piperidin-4-yl)methyl)piperidin-4-yl)-1-oxoisoindolin-2-yl)piperidine-2,6-dione

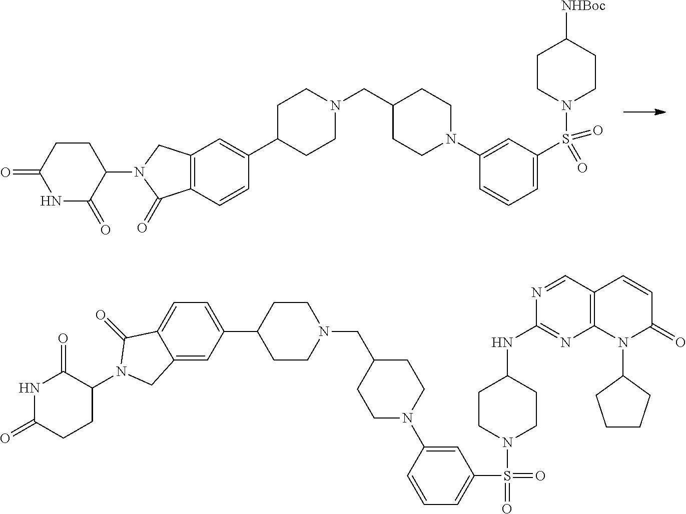 [SMILES: CC(C)(C)OC(=O)NC1CCN(S(=O)(=O)c2cccc(N3CCC(CN4CCC(c5ccc6c(c5)CN(C5CCC(=O)NC5=O)C6=O)CC4)CC3)c2)CC1 ; O=C1CCC(N2Cc3cc(C4CCN(CC5CCN(c6cccc(S(=O)(=O)N7CCC(Nc8ncc9ccc(=O)n(C%10CCCC%10)c9n8)CC7)c6)CC5)CC4)ccc3C2=O)C(=O)N1]

tert-butyl (1-((3-(1-((1-(2-(2,6-Dioxopiperidin-3-yl)-1-oxoisoindolin-5-yl)piperidin-4-yl)methyl)piperidin-4-yl)phenyl)sulfonyl)piperidin-4-yl)carbamate was converted to the title compound by proceeding analogously as described in Example 7, Steps 5-6. The resulting mixture was purified by Prep-TLC to give the title compound (15.2 mg, 16.6%) as a white solid [M+1]⁺=876.42.

### Example 36

### Synthesis of 3-(5-(4-((1-(3-((4-((8-cyclopentyl-7-oxo-7,8-dihydropyrido[2,3-d]pyrimidin-2-yl)-amino)piperidin-1-yl)sulfonyl)phenyl)piperidin-4-yl)methyl)piperazin-1-yl)-1-oxoisoindolin-2-yl)piperidine-2,6-dione

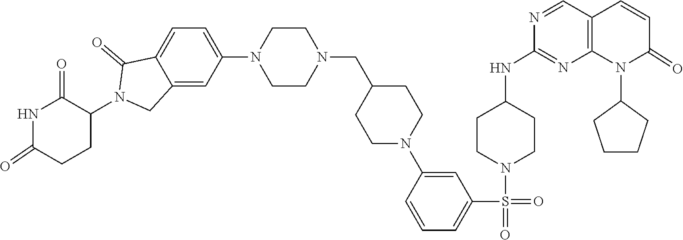 [SMILES: O=C1CCC(N2Cc3cc(N4CCN(CC5CCN(c6cccc(S(=O)(=O)N7CCC(Nc8ncc9ccc(=O)n(C%10CCCC%10)c9n8)CC7)c6)CC5)CC4)ccc3C2=O)C(=O)N1]

### Step 1: benzyl 4-(dimethoxymethyl)piperidine-1-carboxylate

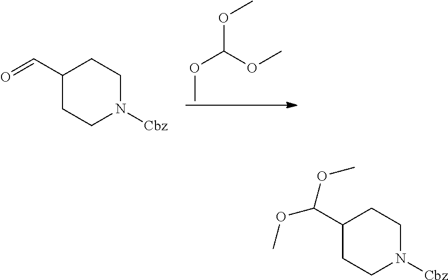

To a mixture of benzyl 4-formylpiperidine-1-carboxylate (1.00 g, 2.40 mmol, 1.00 eq.) in MeOH (9.0 mL) was added p-TsOH (38 mg, 0.20 mmol, 0.05 eq.) and trimethoxymethane (2.14 g, 20.22 mmol, 5.00 eq.). The mixture was stirred at RT for 12 h and then extracted with EtOAc. Purification of the crude product by silica gel column chromatography eluting with PE/EtOAc (10:1) gave the title compound (948 mg, 80.3%) as a colorless oil.

### Step 2: 4-(dimethoxymethyl)piperidine

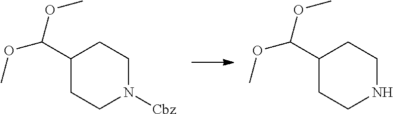 [SMILES: COC(OC)C1CCN(C(=O)OCc2ccccc2)CC1 ; COC(OC)C1CCNCC1]

To a mixture of benzyl 4-(dimethoxymethyl) piperidine-1-carboxylate (948 mg, 3.23 mmol, 1.00 eq.) in MeOH (10.0 mL) was added Pd/C (400 mg) and the reaction mixture was stirred at RT under H₂ for overnight. The resulting mixture was filtered through Celite and the filtrate was concentrated to give the title compound (520 mg, crude) as a colorless oil.

### Step 3: tert-butyl (1-((3-(4-(dimethoxymethyl) piperidin-1-yl)phenyl)sulfonyl)piperidin-4-yl)-carbamate

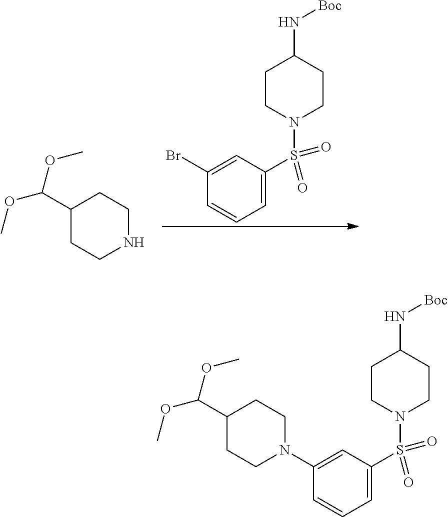 [SMILES: COC(OC)C1CCNCC1 ; CC(C)(C)OC(=O)NC1CCN(S(=O)(=O)c2cccc(Br)c2)CC1 ; COC(OC)C1CCN(c2cccc(S(=O)(=O)N3CCC(NC(=O)OC(C)(C)C)CC3)c2)CC1]

A mixture of 4-(dimethoxymethyl) piperidine (100 mg, 0.63 mmol, 1.20 eq.), K2CO₃ (215 mg, 1.56 mmol, 3.00 eq.), CuI (20 mg, 0.104 mmol, 0.20 eq.), L-proline (18 mg, 0.16 mmol, 0.30 eq.) and tert-butyl (1-((3-bromophenyl)sulfonyl)piperidin-4-yl)carbamate (219 mg, 0.52 mmol, 1.00 eq.) in DMSO (4.0 mL) was stirred at 90° C. overnight. The reaction mixture was extracted with EtOAc and purified by silica gel column chromatography eluting with PE/EtOAc (1:1) to give the title compound (98 mg, 38.0%) as white solid.

### Step 4: tert-butyl (1-((3-(4-formylpiperidin-1-yl)phenyl)sulfonyl)piperidin-4-yl)carbamate

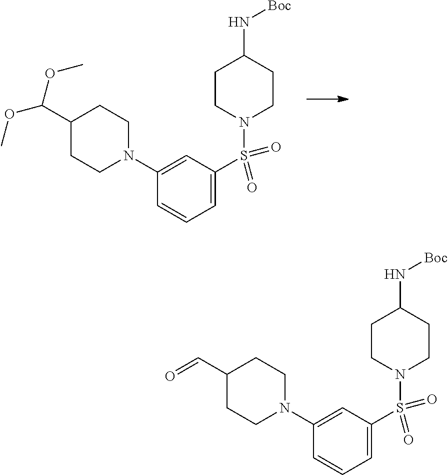 [SMILES: COC(OC)C1CCN(c2cccc(S(=O)(=O)N3CCC(NC(=O)OC(C)(C)C)CC3)c2)CC1 ; CC(C)(C)OC(=O)NC1CCN(S(=O)(=O)c2cccc(N3CCC(C=O)CC3)c2)CC1]

To a mixture of tert-butyl (1-((3-(4-(dimethoxymethyl)piperidin-1-yl)phenyl)sulfonyl) piperidin-4-yl)carbamate (640 mg, 1.29 mmol, 1.00 eq.) in DCM (4.0 mL) was added TFA (4.0 mL) and the mixture was stirred at 45° C. overnight. The reaction mixture was concentrated and dissolved in DCM (5.0 mL) and TEA (261 mg, 2.58 mmol, 2.00 eq.) and (Boc)₂O (562 mg, 2.58 mmol, 2.00 eq.) were added to above solution. The solution was stirred at RT for 4 h, extracted with DCM and the crude product was purified by silica gel column chromatography eluting with PE/EtOAc (3:1) to give title compound (400 mg, 68.7%) as yellow solid.

### Step 5: tert-butyl (1-((3-(4-((4-(2-(2,6-dioxopiperidin-3-yl)-1-oxoisoindolin-5-yl)piperazin-1-yl)-methyl)piperidin-1-yl)phenyl)sulfonyl)piperidin-4-yl)carbamate

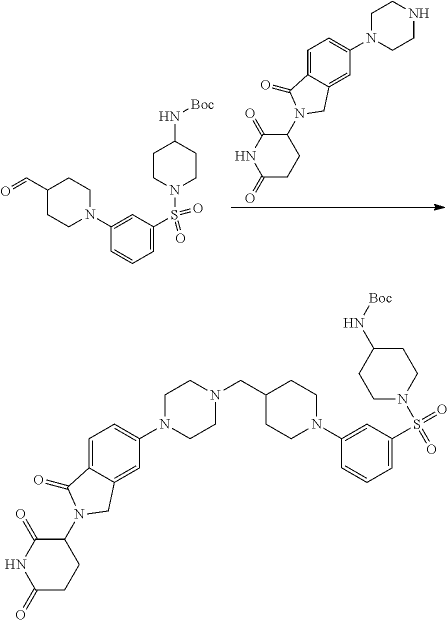 [SMILES: CC(C)(C)OC(=O)NC1CCN(S(=O)(=O)c2cccc(N3CCC(C=O)CC3)c2)CC1 ; O=C1CCC(N2Cc3cc(N4CCNCC4)ccc3C2=O)C(=O)N1 ; CC(C)(C)OC(=O)NC1CCN(S(=O)(=O)c2cccc(N3CCC(CN4CCN(c5ccc6c(c5)CN(C5CCC(=O)NC5=O)C6=O)CC4)CC3)c2)CC1]

The compound was prepared analogously as described in Example 35, Step 5. The reaction mixture was extracted with DCM and purified by silica gel column chromatography eluting with DCM/MeOH (20:1) to give the title compound (114 mg, 65.1%) as a yellow solid.

### Step 6: 3-(5-(4-((1-(3-((4-((8-cyclopentyl-7-oxo-7,8-dihydropyrido[2,3-d]pyrimidin-2-yl)-amino)piperidin-1-yl)sulfonyl)phenyl)piperidin-4-yl)methyl)piperazin-1-yl)-1-oxoisoindolin-2-yl)piperidine-2,6-dione

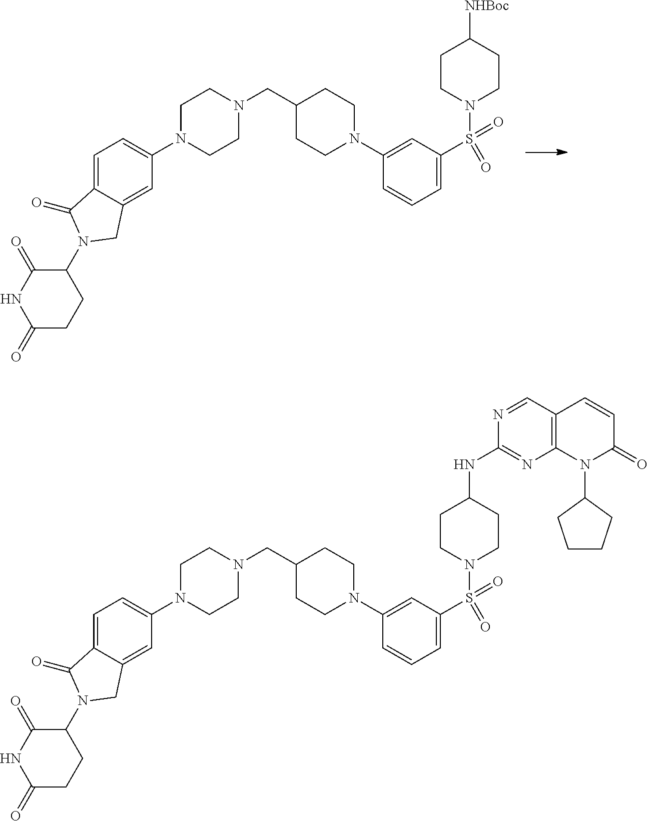 [SMILES: CC(C)(C)OC(=O)NC1CCN(S(=O)(=O)c2cccc(N3CCC(CN4CCN(c5ccc6c(c5)CN(C5CCC(=O)NC5=O)C6=O)CC4)CC3)c2)CC1 ; O=C1CCC(N2Cc3cc(N4CCN(CC5CCN(c6cccc(S(=O)(=O)N7CCC(Nc8ncc9ccc(=O)n(C%10CCCC%10)c9n8)CC7)c6)CC5)CC4)ccc3C2=O)C(=O)N1]

tert-butyl (1-((3-(4-((4-(2-(2,6-Dioxopiperidin-3-yl)-1-oxoisoindolin-5-yl)piperazin-1-yl)methyl)piperidin-1-yl)phenyl)sulfonyl)piperidin-4-yl)carbamate was converted to the title compound by proceeding analogously as described in Example 7, Steps 5-6. The crude product was purified by prep-TLC to give the title compound (13 mg, 9.9%) as pale yellow solid.

MS(ES, m/z): [M+1]⁺=877.6.

### Example 37

### Synthesis of 3-(5-(1-(3-((4-((8-cyclopentyl-7-oxo-7,8-dihydropyrido[2,3-d]pyrimidin-2-yl)amino)piperidin-1-yl)sulfonyl)benzyl)azetidin-3-yl)-1-oxoisoindolin-2-yl)piperidine-2,6-dione

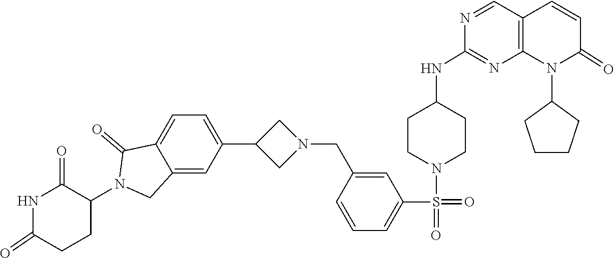 [SMILES: O=C1CCC(N2Cc3cc(C4CN(Cc5cccc(S(=O)(=O)N6CCC(Nc7ncc8ccc(=O)n(C9CCCC9)c8n7)CC6)c5)C4)ccc3C2=O)C(=O)N1]

### Step 1: (1-(tert-butoxycarbonyl)azetidin-3-yl)zinc(II) iodide

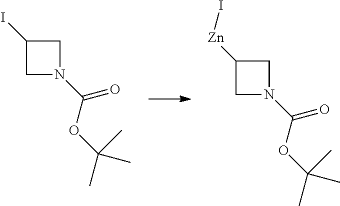

To a mixture of Zn dust (300 mg, 4.59 mmol, 1.30 eq.) in DMA (3.0 mL) was added 1,2-dibromoethene (66 mg, 0.35 mmol, 0.10 eq.) and the mixture was stirred at 65° C. under N₂ for 30 min. The mixture was allowed to cool to RT and TMSCl (38 mg, 0.35 mmol, 0.10 eq.) was added. After stirring the mixture for 30 min., a solution of tert-butyl 3-iodoazetidine-1-carboxylate (1.00 g, 3.53 mmol, 1.00 eq.) in DMA (1.0 mL) was added dropwise. The mixture was stirred at 65° C. under N₂ for 2 h, cooled to RT and used in next step without further purification.

### Step 2: tert-butyl 3-(2-(2,6-dioxopiperidin-3-yl)-1-oxoisoindolin-5-yl)azetidine-1-carboxylate

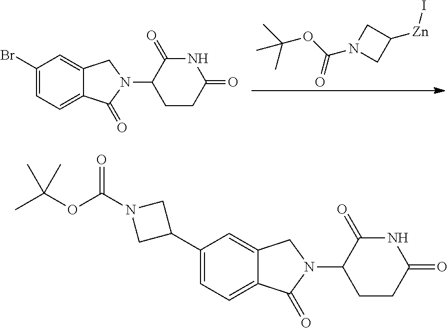

A mixture of 3-(5-bromo-1-oxoisoindolin-2-yl)piperidine-2,6-dione (185 mg, 0.57 mmol, 1.00 eq.) in DMA (2.0 mL) was added CuI (12 mg, 0.06 mmol, 0.10 eq.), Pd(dppf)Cl₂ (44 mg, 0.06 mmol, 0.10 eq.). A solution of (1-(tert-butoxycarbonyl)azetidin-3-yl)zinc(II) iodide (600 mg, 1.72 mmol, 3.00 eq.) in DMA was slowly added and the mixture was stirred at 90° C. under N2 overnight. The mixture was concentrated and purified by column chromatography on silica gel (EA) to give tert-butyl 3-(2-(2,6-dioxopiperidin-3-yl)-1-oxoisoindolin-5-yl)azetidine-1-carboxylate (150 mg, 65.8%) as a brown solid.

### Step 3: 3-(5-(azetidin-3-yl)-1-oxoisoindolin-2-yl)piperidine-2,6-dione

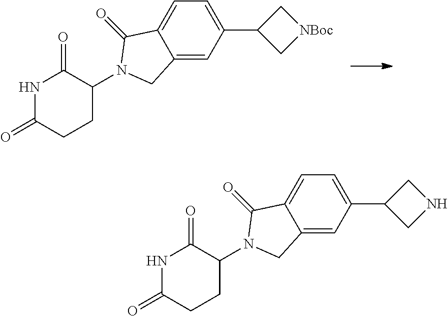 [SMILES: CC(C)(C)OC(=O)N1CC(c2ccc3c(c2)CN(C2CCC(=O)NC2=O)C3=O)C1 ; O=C1CCC(N2Cc3cc(C4CNC4)ccc3C2=O)C(=O)N1]

To a solution of tert-butyl 3-(2-(2,6-dioxopiperidin-3-yl)-1-oxoisoindolin-5-yl)azetidine-1-carboxylate (44 mg, 0.11 mmol, 1.00 eq.) in DCM (1.0 mL) was added TFA (0.2 mL) dropwise and the solution was stirred at RT for 3 h. The resulting mixture was concentrated to give the crude product (40.0 mg, 100%) as a brown oil, which was used to next step without further purification.

### Step 4: 3-(5-(1-(3-((4-((8-cyclopentyl-7-oxo-7,8-dihydropyrido[2,3-d]pyrimidin-2-yl)amino)-piperidin-1-yl)sulfonyl)benzyl)azetidin-3-yl)-1-oxoisoindolin-2-yl)piperidine-2,6-dione

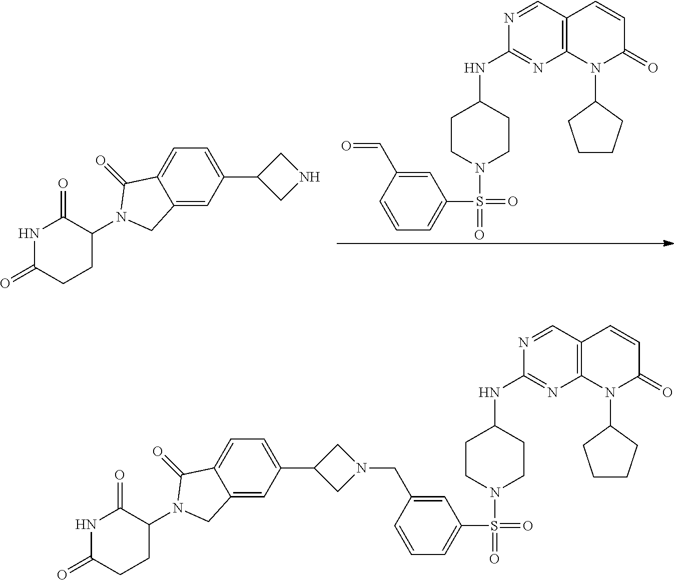 [SMILES: O=C1CCC(N2Cc3cc(C4CNC4)ccc3C2=O)C(=O)N1 ; O=Cc1cccc(S(=O)(=O)N2CCC(Nc3ncc4ccc(=O)n(C5CCCC5)c4n3)CC2)c1 ; O=C1CCC(N2Cc3cc(C4CN(Cc5cccc(S(=O)(=O)N6CCC(Nc7ncc8ccc(=O)n(C9CCCC9)c8n7)CC6)c5)C4)ccc3C2=O)C(=O)N1]

To a solution of 3-(5-(azetidin-3-yl)-1-oxoisoindolin-2-yl)piperidine-2,6-dione (40 mg, 0.11 mmol, 1.00 eq.) in THF (1.0 mL) and DMF (1.0 mL) were added TEA (11.1 mg, 0.11 mmol, 1.00 eq.), AcOH (6.6 mg, 0.11 mmol, 1.00 eq.), and 3-((4-((8-cyclopentyl-7-oxo-7,8-dihydropyrido[2,3-d]pyrimidin-2-yl)amino)piperidin-1-yl)sulfonyl)benzaldehyde (63.5 mg, 0.132 mmol, 1.20 eq.). The solution was stirred at 40° C. for 1 h and cooled to RT. NaBH₃CN (21.0 mg, 0.33 mmol, 3.00 eq.)) was added and the mixture was stirred at RT for 16 h. The resulting mixture was diluted with water and extracted with EtOAc. The combined organic layer was washed with brine, dried over anhydrous Na₂SO₄ and concentrated. The residue was purified by TLC, eluted with DCM/MeOH (20:1), to afford the title compound (10.0 mg, 11.9%) as a white solid. MS (ES, m/z): [M+1]⁺=765.4.

### Example 38

### Synthesis of 3-(5-(4-((4-(3-((4-((8-cyclopentyl-7-oxo-7,8-dihydropyrido[2,3-d]pyrimidin-2-yl)amino)piperidin-1-yl)sulfonyl)phenyl)piperazin-1-yl)methyl)piperidin-1-yl)-1-oxoisoindolin-2-yl)piperidine-2,6-dione

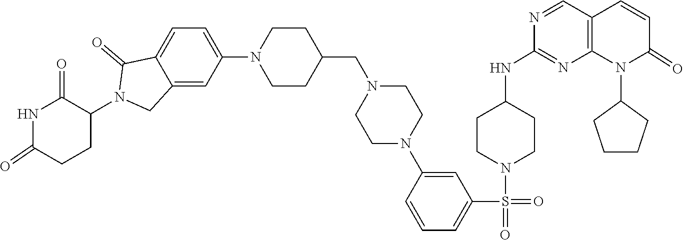 [SMILES: O=C1CCC(N2Cc3cc(N4CCC(CN5CCN(c6cccc(S(=O)(=O)N7CCC(Nc8ncc9ccc(=O)n(C%10CCCC%10)c9n8)CC7)c6)CC5)CC4)ccc3C2=O)C(=O)N1]

### Step 1: benzyl 4-(3-((4-((tert-butoxycarbonyl)amino)piperidin-1-yl)sulfonyl)phenyl)piperazine-1-carboxylate

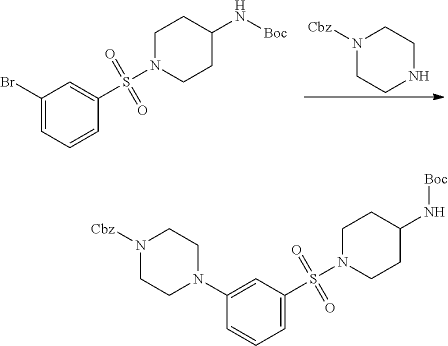 [SMILES: CC(C)(C)OC(=O)NC1CCN(S(=O)(=O)c2cccc(Br)c2)CC1 ; O=C(OCc1ccccc1)N1CCNCC1 ; CC(C)(C)OC(=O)NC1CCN(S(=O)(=O)c2cccc(N3CCN(C(=O)OCc4ccccc4)CC3)c2)CC1]

A mixture of tert-butyl (1-((3-bromophenyl)sulfonyl)piperidin-4-yl)carbamate (5.00 g, 11.96 mmol, 1.00 eq.), K2C03 (5.78 g, 41.86 mmol, 3.50 eq.), CuI (0.45 g, 2.39 mmol, 0.20 eq.), L-PRO (0.41 g, 3.59 mmol, 0.30 eq.) in DMSO (25.00 mL) and benzyl piperazine-1-carboxylate (3.43 g, 15.55 mmol, 1.30 eq.) was stirred at 100° C. for 12 h and then quenched with H₂O and extracted with EtOAc. The organic layer was concentrated and the residue was purified by silica gel column chromatography eluting with PE/EtOAc (3:1) to give the title compound (1.96 g, 29.4%) as white solid.

### Step 2: tert-butyl (1-((3-(piperazin-1-yl)phenyl)sulfonyl)piperidin-4-yl)carbamate

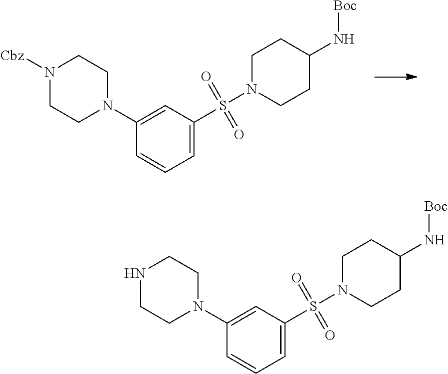 [SMILES: CC(C)(C)OC(=O)NC1CCN(S(=O)(=O)c2cccc(N3CCN(C(=O)OCc4ccccc4)CC3)c2)CC1 ; CC(C)(C)OC(=O)NC1CCN(S(=O)(=O)c2cccc(N3CCNCC3)c2)CC1]

To a stirred solution of 1-benzhydrylazetidin-3-ol (2.53 g, 4.53 mmol, 1.00 eq.) in MeOH (20.0 mL) and THF (3.00 mL) was added Pd(OH)₂ (1.00 g). The resulting mixture was stirred at 50° C. under H₂ (50 psi) for 12 h. The mixture was filtered and concentrated to afford the crude product (1.86 g, crude) as a white solid.

### Step 3: benzyl 4-((4-(3-((4-((tert-butoxycarbonyl)amino)piperidin-1-yl)sulfonyl)phenyl)piperazin-1-yl)methyl)piperidine-1-carboxylate

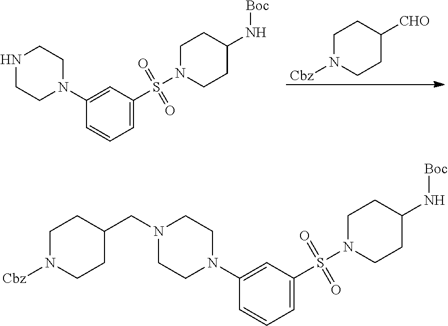 [SMILES: CC(C)(C)OC(=O)NC1CCN(S(=O)(=O)c2cccc(N3CCNCC3)c2)CC1 ; O=CC1CCN(C(=O)OCc2ccccc2)CC1 ; CC(C)(C)OC(=O)NC1CCN(S(=O)(=O)c2cccc(N3CCN(CC4CCN(C(=O)OCc5ccccc5)CC4)CC3)c2)CC1]

To a solution or tert-butyl (1-((3-piperazin-1-yl)phenyl)sulfonyl)piperidin-4-ylcarbamate (1.07 g, 2.52 mmol, 1.00 eq.) in MeOH (10.0 mL) were added AcOH (3 drops) and benzyl 4-formylpiperidine-1-carboxylate (933 mg, 3.78 mmol, 1.50 eq.). The solution was stirred at 45° C. for 1 h. The solution cooled to RT and NaBH₃CN (475 mg, 7.56 mmol, 3.00 eq.) was added, The mixture was stirred at RT for 12 h and then diluted with water and extracted with EtOAc. The combined organic layer was washed with brine, dried over anhydrous Na₂SO₄, and concentrated. The residue was purified by silica gel column chromatography eluting with DCM/MeOH (60:1) to give the title compound (830 mg, 50.3%) as white solid.

### Step 4: tert-butyl (1-((3-(4-((1-(2-(2,6-dioxopiperidin-3-yl)-1-oxoisoindolin-5-yl)piperidin-4-yl)methyl)piperazin-1-yl)phenyl)sulfonyl)piperidin-4-yl)carbamate

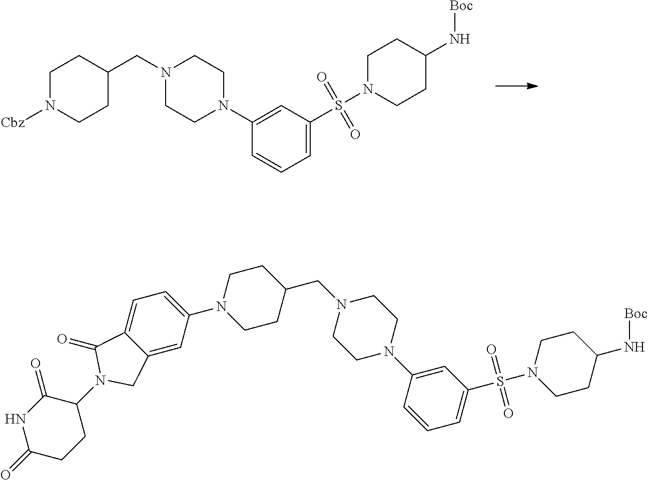 [SMILES: CC(C)(C)OC(=O)NC1CCN(S(=O)(=O)c2cccc(N3CCN(CC4CCN(C(=O)OCc5ccccc5)CC4)CC3)c2)CC1 ; CC(C)(C)OC(=O)NC1CCN(S(=O)(=O)c2cccc(N3CCN(CC4CCN(c5ccc6c(c5)CN(C5CCC(=O)NC5=O)C6=O)CC4)CC3)c2)CC1]

Benzyl 4-((4-(3-((4-((tert-butoxycarbonyl)amino)piperidin-1-yl)sulfonyl)phenyl)piperazin-1-yl)methyl)piperidine-1-carboxylate was converted to the title compound using similar procedure as described in Example 34, Step 4-7. The residue was purified by silica gel column chromatography, eluted with DCM/MeOH (40:1), to afford the title compound (92 mg, 60.5%) as a yellow solid.

### Step 5: 3-(5-(4-((4-(3-((4-((8-cyclopentyl-7-oxo-7,8-dihydropyrido[2,3-d]pyrimidin-2-yl)-amino)piperidin-1-yl)sulfonyl)phenyl)piperazin-1-yl)methyl)piperidin-1-yl)-1-oxoisoindolin-2-yl)piperidine-2,6-dione

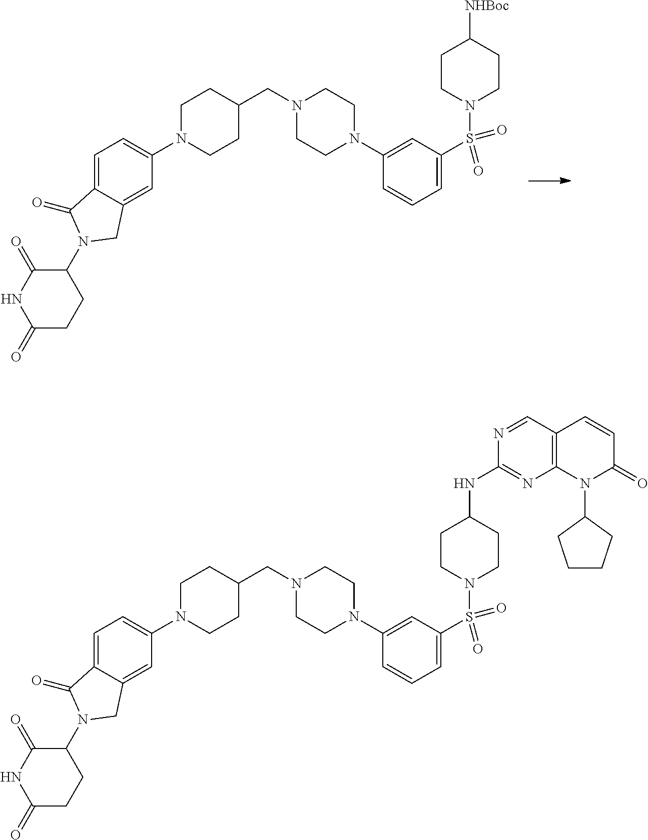 [SMILES: CC(C)(C)OC(=O)NC1CCN(S(=O)(=O)c2cccc(N3CCN(CC4CCN(c5ccc6c(c5)CN(C5CCC(=O)NC5=O)C6=O)CC4)CC3)c2)CC1 ; O=C1CCC(N2Cc3cc(N4CCC(CN5CCN(c6cccc(S(=O)(=O)N7CCC(Nc8ncc9ccc(=O)n(C%10CCCC%10)c9n8)CC7)c6)CC5)CC4)ccc3C2=O)C(=O)N1]

tert-butyl (1-((3-(4-((1-(2-(2,6-Dioxopiperidin-3-yl)-1-oxoisoindolin-5-yl)piperidin-4-yl)methyl)piperazin-1-yl)phenyl)sulfonyl)piperidin-4-yl)carbamate was converted to the title compound by proceeding analogously as described in Example 7, Steps 5-6. The residue was purified by TLC, eluted with DCM/MeOH (20:1), to afford the title compound (30.5 mg, 29.0%) as a pale-yellow solid. MS (ES, m/z): [M+1]⁺=877.

### Example 39

### Synthesis of 3-(4-(1′-(3-((4-((8-cyclopentyl-7-oxo-7,8-dihydropyrido[2,3-d]pyrimidin-2-yl)amino)piperidin-1-yl)sulfonyl)phenyl)-[1,4′-bipiperidin]-4-yl)-3-methyl-2-oxo-2,3-dihydro-1H-benzo[d]imidazol-1-yl)piperidine-2,6-dione

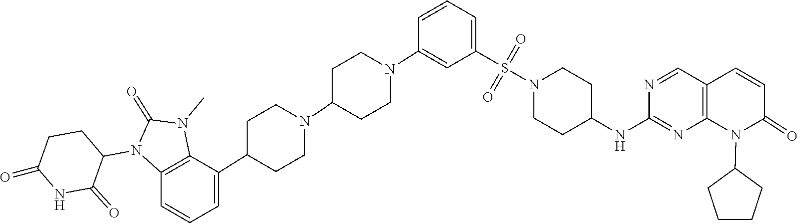 [SMILES: Cn1c(=O)n(C2CCC(=O)NC2=O)c2cccc(C3CCN(C4CCN(c5cccc(S(=O)(=O)N6CCC(Nc7ncc8ccc(=O)n(C9CCCC9)c8n7)CC6)c5)CC4)CC3)c21]

### Step 1: tert-butyl (1-((3-(1,4-dioxa-8-azaspiro[4.5]decan-8-yl)phenyl)sulfonyl)piperidin-4-yl)-carbamate

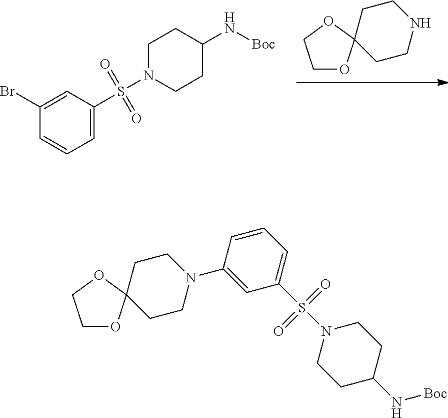 [SMILES: CC(C)(C)OC(=O)NC1CCN(S(=O)(=O)c2cccc(Br)c2)CC1 ; C1CC2(CCN1)OCCO2 ; CC(C)(C)OC(=O)NC1CCN(S(=O)(=O)c2cccc(N3CCC4(CC3)OCCO4)c2)CC1]

A mixture of tert-butyl (1-((3-bromophenyl)sulfonyl)piperidin-4-yl)carbamate (1.00 g, 2.40 mmol, 1.00 eq.), K₂CO₃ (1.16 g, 8.40 mmol, 3.50 eq.), CuI (91 mg, 0.480 mmol, 0.20 eq.), L-proline (83 mg, 0.72 mmol, 0.30 eq.) and 1,4-dioxa-8-azaspiro[4.5]decane (412 mg, 2.88 mmol, 1.20 eq.) in DMSO (10.0 mL) was stirred at 90° C. overnight. The reaction mixture was extracted with DCM and purified by silica gel column chromatography eluting with PE/EtOAc (1:1) to give the title compound (624 mg, 54.3%) as yellow solid.

### Step 2: tert-butyl (1-((3-(4-oxopiperidin-1-yl)phenyl)sulfonyl)piperidin-4-yl)carbamate

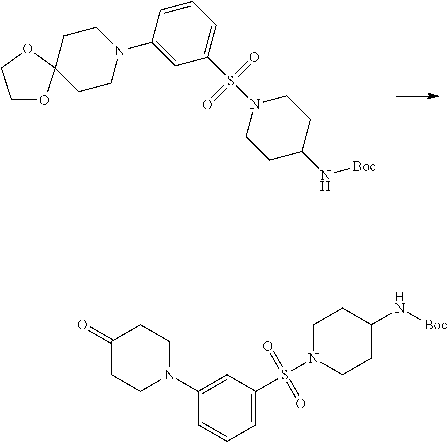 [SMILES: CC(C)(C)OC(=O)NC1CCN(S(=O)(=O)c2cccc(N3CCC4(CC3)OCCO4)c2)CC1 ; CC(C)(C)OC(=O)NC1CCN(S(=O)(=O)c2cccc(N3CCC(=O)CC3)c2)CC1]

To a solution of tert-butyl (1-((3-(1,4-dioxa-8-azaspiro[4.5]decan-8-yl)phenyl)-sulfonyl)piperidin-4-yl)carbamate (624 mg, 1.30 mmol, 1.00 eq.) in acetone (6.0 mL) and H₂O (12.0 mL) was added TsOH·H₂O (49 mg, 0.26 mmol, 0.20 eq.). The reaction mixture was stirred at 60° C. overnight. The mixture was extracted with DCM and purified by silica gel column chromatography eluting PE/EtOAc (1:1) to give the title compound (450 mg, 78.7%) as yellow solid.

### Step 3: tert-butyl (1-((3-(4-(1-(2,6-dioxopiperidin-3-yl)-3-methyl-2-oxo-2,3-dihydro-1H-benzo[d]imidazol-4-yl)-[1,4′-bipiperidin]-1′-yl)phenyl)sulfonyl)piperidin-4-yl)carbamate

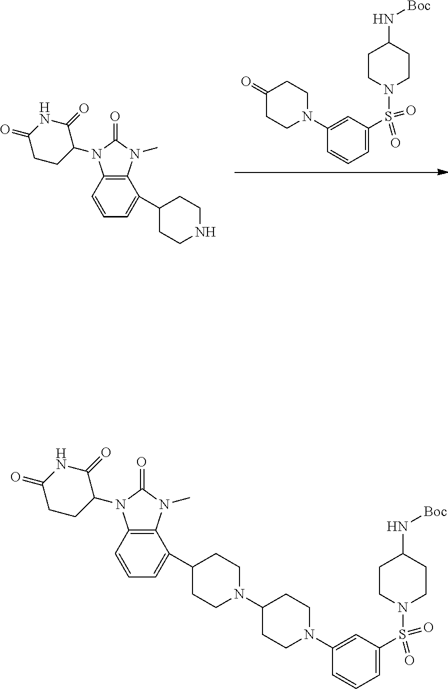 [SMILES: Cn1c(=O)n(C2CCC(=O)NC2=O)c2cccc(C3CCNCC3)c21 ; CC(C)(C)OC(=O)NC1CCN(S(=O)(=O)c2cccc(N3CCC(=O)CC3)c2)CC1 ; Cn1c(=O)n(C2CCC(=O)NC2=O)c2cccc(C3CCN(C4CCN(c5cccc(S(=O)(=O)N6CCC(NC(=O)OC(C)(C)C)CC6)c5)CC4)CC3)c21]

A mixture of 3-(3-methyl-2-oxo-4-(piperidin-4-yl)-2,3-dihydro-1H-benzo[d]imidazol-1-yl)piperidine-2,6-dione (387 mg, 1.13 mmol, 1.00 eq.) in THF (5.0 mL) was added tert-butyl (1-((3-(4-oxopiperidin-1-yl)phenyl)sulfonyl)piperidin-4-yl)carbamate (44 mg, 1.02 mmol, 0.90 eq.) and 1 drop of AcOH and the reaction mixture was stirred at 40° C. for 0.5 h. NaBH₃CN (142 mg, 2.60 mmol, 2.00 eq.) was added at RT and stirred at RT overnight. The reaction mixture was extracted with DCM and purified by silica gel column chromatography eluting with DCM/MeOH (10:1) to give the title compound (200 mg, 23.2%) as a yellow solid.

### Step 4: 3-(4-(1′-(3-((4-((8-cyclopentyl-7-oxo-7,8-dihydropyrido[2,3-d]pyrimidin-2-yl)amino)-piperidin-1-yl)sulfonyl)phenyl)-[1,4′-bipiperidin]-4-yl)-3-methyl-2-oxo-2,3-dihydro-1H-benzo[d]imidazol-1-yl)piperidine-2,6-dione

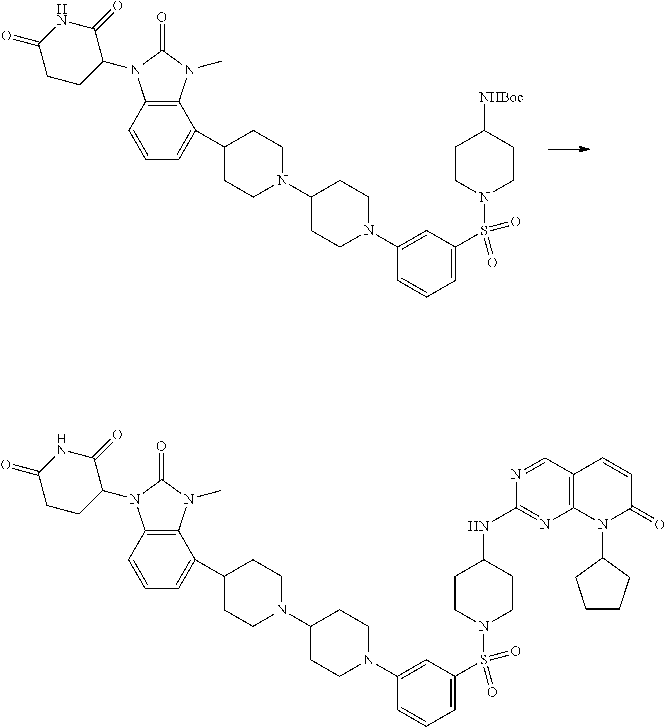 [SMILES: Cn1c(=O)n(C2CCC(=O)NC2=O)c2cccc(C3CCN(C4CCN(c5cccc(S(=O)(=O)N6CCC(NC(=O)OC(C)(C)C)CC6)c5)CC4)CC3)c21 ; Cn1c(=O)n(C2CCC(=O)NC2=O)c2cccc(C3CCN(C4CCN(c5cccc(S(=O)(=O)N6CCC(Nc7ncc8ccc(=O)n(C9CCCC9)c8n7)CC6)c5)CC4)CC3)c21]

tert-butyl (1-((3-(4-(1-(2,6-Dioxopiperidin-3-yl)-3-methyl-2-oxo-2,3-dihydro-1H-benzo[d]imidazol-4-yl)-[1,4′-bipiperidin]-1′-yl)phenyl)sulfonyl)piperidin-4-yl)carbamate was converted to the title compound by proceeding analogously as described in Example 7, Steps 5-6.

MS (ES, m/z): [M+1]⁺=877.4.

### Example 40

### Synthesis of 3-(4-(1-(3-((4-((8-cyclopentyl-7-oxo-7,8-dihydropyrido[2,3-d]pyrimidin-2-yl)-amino)piperidin-1-yl)sulfonyl)benzyl)azetidin-3-yl)-3-methyl-2-oxo-2,3-dihydro-1H-benzo[d]imidazol-1-yl)piperidine-2,6-dione

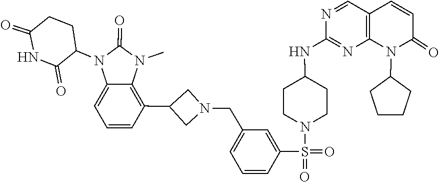 [SMILES: Cn1c(=O)n(C2CCC(=O)NC2=O)c2cccc(C3CN(Cc4cccc(S(=O)(=O)N5CCC(Nc6ncc7ccc(=O)n(C8CCCC8)c7n6)CC5)c4)C3)c21]

### Step 1: tert-butyl (1-((3-cyanophenyl)sulfonyl)piperidin-4-yl)carbamate

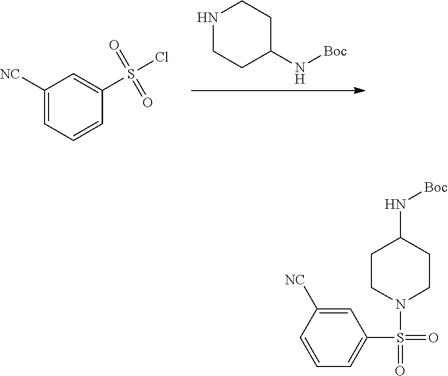 [SMILES: N#Cc1cccc(S(=O)(=O)Cl)c1 ; CC(C)(C)OC(=O)NC1CCNCC1 ; CC(C)(C)OC(=O)NC1CCN(S(=O)(=O)c2cccc(C#N)c2)CC1]

To a stirred solution of tert-butyl piperidin-4-ylcarbamate (5.00 g, 2.50 mmol, 1.00 eq.) in THF (60.00 mL) were added TEA (6.31 g, 6.24 mmol, 2.50 eq.) and 3-cyanobenzenesulfonyl chloride (5.28 g, 2.62 mmol, 1.05 eq.) in THF (40.00 mL) at 0° C. The resulting mixture was stirred at RT for 12 h, quenched with H₂O and then extracted with DCM. The organic layer was concentrated and the solid was washed by PE to give the title compound (8.36 g, 91.9%) as white solid.

### Step 2: 3-((4-aminopiperidin-1-yl)sulfonyl)benzonitrile

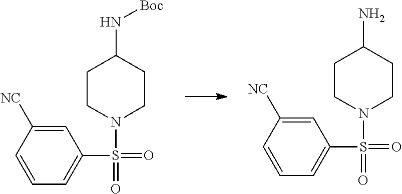 [SMILES: CC(C)(C)OC(=O)NC1CCN(S(=O)(=O)c2cccc(C#N)c2)CC1 ; N#Cc1cccc(S(=O)(=O)N2CCC(N)CC2)c1]

To a solution of tert-butyl (1-((3-cyanophenyl)sulfonyl)piperidin-4-yl)carbamate (2.00 g, 5.48 mmol, 1.00 eq.) in DCM (20.0 mL) was added TFA (5.0 mL) dropwise and the solution was stirred at RT for 3 h. The resulting mixture was concentrated to give the crude product (1.50 g, 100%) as a yellow oil, which was used to next step without further purification.

### Step 3: 3-((4-((8-cyclopentyl-7-oxo-7,8-dihydropyrido[2,3-d]pyrimidin-2-yl)amino)piperidin-1-yl)sulfonyl)benzonitrile

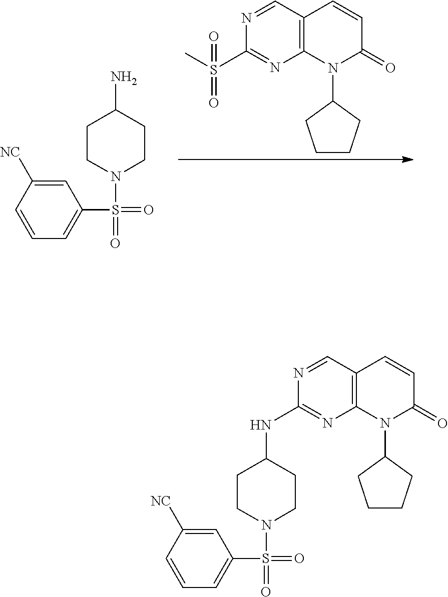 [SMILES: N#Cc1cccc(S(=O)(=O)N2CCC(N)CC2)c1 ; CS(=O)(=O)c1ncc2ccc(=O)n(C3CCCC3)c2n1 ; N#Cc1cccc(S(=O)(=O)N2CCC(Nc3ncc4ccc(=O)n(C5CCCC5)c4n3)CC2)c1]

To a solution of 3-((4-aminopiperidin-1-yl)sulfonyl)benzonitrile (1.50 g, 5.48 mmol, 1.00 eq.) in DMSO (15.0 mL) were added DIEA (2.12 g, 16.44 mmol, 3.00 eq.) and 8-cyclopentyl-2-(methylsulfonyl)pyrido[2,3-d]pyrimidin-7 (8H)-one (1.93 g, 6.58 mmol, 1.20 eq.). The resulting mixture was stirred for 16 h at 65° C. under nitrogen atmosphere, cooled, diluted with water, and then extracted with EtOAc. The combined organic layer was washed with water, dried over anhydrous Na₂SO₄, filtered, and then concentrated. The residue was purified by silica gel column chromatography, eluted with DCM/MeOH (70:1), to afford the title compound (1.3 g, 50.0%) as a yellow solid.

### Step 4: 3-((4-((8-cyclopentyl-7-oxo-7,8-dihydropyrido[2,3-d]pyrimidin-2-yl)amino)piperidin-1-yl)-sulfonyl)benzaldehyde

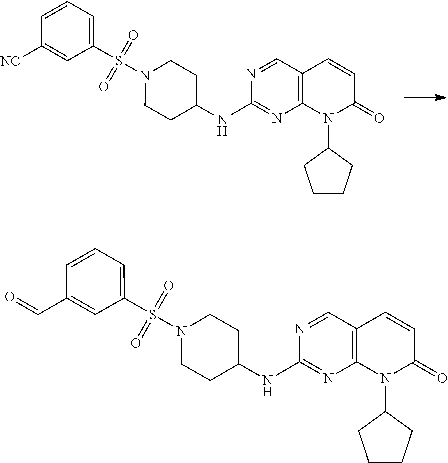 [SMILES: N#Cc1cccc(S(=O)(=O)N2CCC(Nc3ncc4ccc(=O)n(C5CCCC5)c4n3)CC2)c1 ; O=Cc1cccc(S(=O)(=O)N2CCC(Nc3ncc4ccc(=O)n(C5CCCC5)c4n3)CC2)c1]

A mixture of 3-((4-((8-cyclopentyl-7-oxo-7,8-dihydropyrido[2,3-d]pyrimidin-2-yl)amino)-piperidin-1-yl)sulfonyl)benzonitrile (200 mg, 0.42 mmol, 1.00 eq.), NaH₂PO₂·H₂O (134 mg, 1.26 mmol, 3.00 eq.) and Raney Ni (100 mg) in pyridine (4.0 mL), H₂O (2.0 mL) and AcOH (2.0 mL) was stirred for 16 h at 50° C. under nitrogen atmosphere. The resulting mixture was diluted with EtOAc and washed with water, brine, dried over anhydrous Na₂SO₄ and concentrated. The residue was purified by silica gel column chromatography, eluted with DCM/MeOH (80:1), to afford the title compound (120 mg, 59.4%) as a white solid.

### Step 5: tert-butyl 3-(1-(2,6-dioxopiperidin-3-yl)-3-methyl-2-oxo-2,3-dihydro-1H-benzo[d]-imidazol-4-yl)azetidine-1-carboxylate

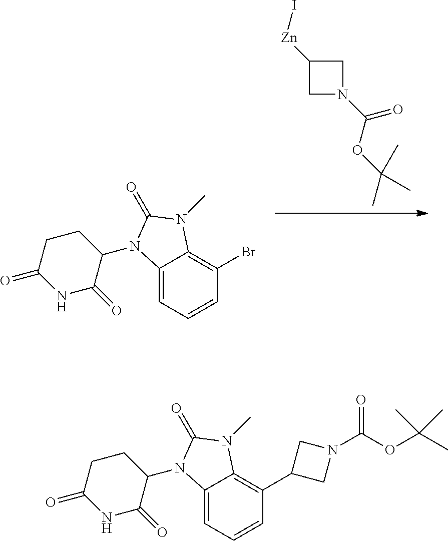

To a mixture of 3-(4-bromo-3-methyl-2-oxo-2,3-dihydro-1H-benzo[d]imidazol-1-yl)piperidine-2,6-dione (193 mg, 0.57 mmol, 1.00 eq.) in DMA (2.0 mL) were added CuI (12 mg, 0.06 mmol, 0.10 eq.) and Pd(dppf)Cl₂ (44 mg, 0.06 mmol, 0.10 eq.). A solution of (1-(tert-butoxycarbonyl)azetidin-3-yl)zinc(II) iodide (600 mg, 1.72 mmol, 3.00 eq.) in DMA was slowly added and the mixture was stirred at 90° C. under N₂ overnight. The mixture was concentrated and purified by column chromatography on silica gel (EA) to the title compound (23 mg, 9.7%) as a yellow solid.

### Step 6: 3-(4-(azetidin-3-yl)-3-methyl-2-oxo-2,3-dihydro-1H-benzo[d]imidazol-1-yl)piperidine-2,6-dione

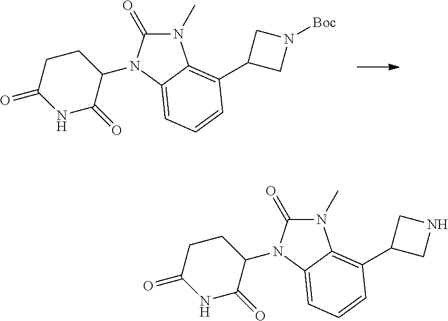 [SMILES: Cn1c(=O)n(C2CCC(=O)NC2=O)c2cccc(C3CN(C(=O)OC(C)(C)C)C3)c21 ; Cn1c(=O)n(C2CCC(=O)NC2=O)c2cccc(C3CNC3)c21]

To a solution of tert-butyl 3-(1-(2,6-dioxopiperidin-3-yl)-3-methyl-2-oxo-2,3-dihydro-1H-benzo[d]imidazol-4-yl)azetidine-1-carboxylate (23 mg, 0.055 mmol, 1.00 eq.) in DCM (1.0 mL) was added TFA (0.2 mL) dropwise and the solution was stirred at RT for 3 h. The resulting mixture was concentrated to give the crude product (20 mg, 100%) as a brown oil, which was used in next step without further purification.

### Step 7: 3-(4-(1-(3-((4-((8-cyclopentyl-7-oxo-7,8-dihydropyrido[2,3-d]pyrimidin-2-yl)amino)-piperidin-1-yl)sulfonyl)benzyl)azetidin-3-yl)-3-methyl-2-oxo-2,3-dihydro-1H-benzo[d]imidazol-1-yl)piperidine-2,6-dione

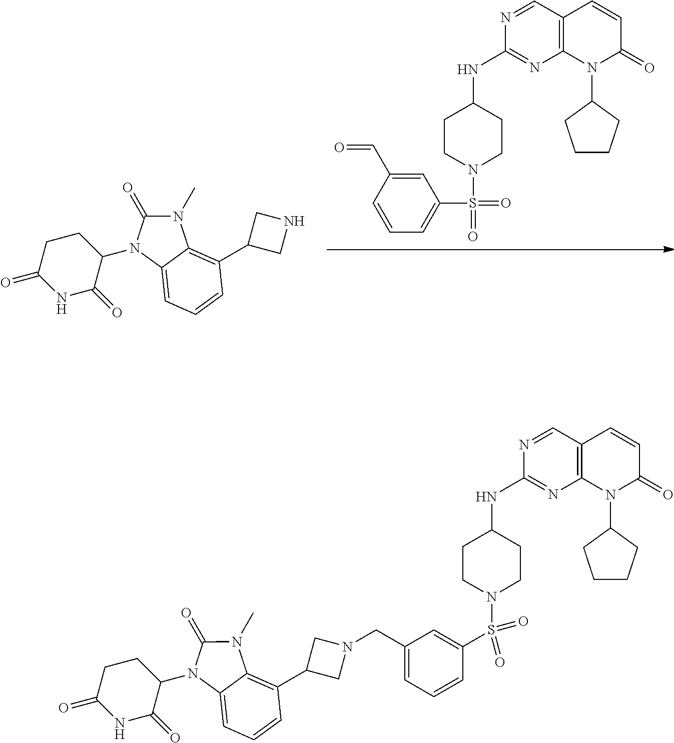 [SMILES: Cn1c(=O)n(C2CCC(=O)NC2=O)c2cccc(C3CNC3)c21 ; O=Cc1cccc(S(=O)(=O)N2CCC(Nc3ncc4ccc(=O)n(C5CCCC5)c4n3)CC2)c1 ; Cn1c(=O)n(C2CCC(=O)NC2=O)c2cccc(C3CN(Cc4cccc(S(=O)(=O)N5CCC(Nc6ncc7ccc(=O)n(C8CCCC8)c7n6)CC5)c4)C3)c21]

To a solution of 3-(4-(azetidin-3-yl)-3-methyl-2-oxo-2,3-dihydro-1H-benzo[d]imidazol-1-yl)piperidine-2,6-dione (20 mg, 0.055 mmol, 1.00 eq.) in THF (1.0 mL) and DMF (1.0 mL) were added TEA (5.6 mg, 0.055 mmol, 1.00 eq.), AcOH (3.3 mg, 0.055 mmol, 1.00 eq.), and 3-((4-((8-cyclopentyl-7-oxo-7,8-dihydropyrido[2,3-d]pyrimidin-2-yl)amino)piperidin-1-yl)sulfonyl)benzaldehyde (31.7 mg, 0.066 mmol, 1.20 eq.). The solution was stirred at 40° C. for 1 h and then cooled to RT. NaBH₃CN (10.4 mg, 0.165 mmol, 3.00 eq.) was added and the mixture was stirred at RT for 16 h. The resulting mixture was diluted with water and extracted with EtOAc. The combined organic layer was washed with brine, dried over anhydrous Na₂SO₄ and concentrated. The residue was purified by TLC, eluted with DCM/MeOH (15:1), to afford the title compound (3.0 mg, 7.1%) as a white solid. MS (ES, m/z): [M+1]⁺=780.4.

### Example 41

### Synthesis of 3-(4-(((2-(3-((4-((8-cyclopentyl-7-oxo-7,8-dihydropyrido[2,3-d]pyrimidin-2-yl)-amino)piperidin-1-yl)sulfonyl)phenoxy)ethyl)amino)methyl)-3-methyl-2-oxo-2,3-dihydro-1H-benzo[d]imidazol-1-yl)piperidine-2,6-dione

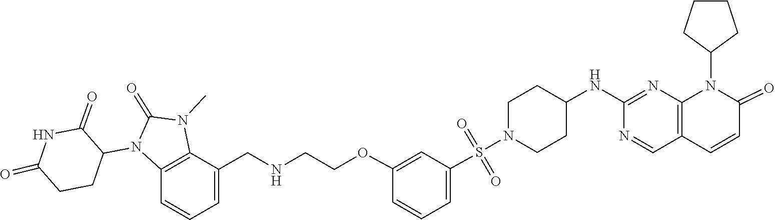 [SMILES: Cn1c(=O)n(C2CCC(=O)NC2=O)c2cccc(CNCCOc3cccc(S(=O)(=O)N4CCC(Nc5ncc6ccc(=O)n(C7CCCC7)c6n5)CC4)c3)c21]

### Step 1: benzyl (2-(3-((4-aminopiperidin-1-yl)sulfonyl)phenoxy)ethyl)carbamate

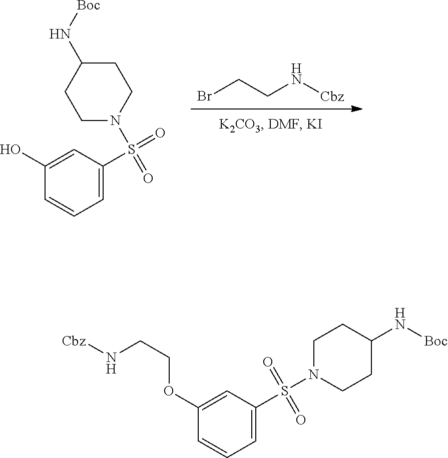 [SMILES: CC(C)(C)OC(=O)NC1CCN(S(=O)(=O)c2cccc(O)c2)CC1 ; O=C(NCCBr)OCc1ccccc1 ; CC(C)(C)OC(=O)NC1CCN(S(=O)(=O)c2cccc(OCCNC(=O)OCc3ccccc3)c2)CC1]

A mixture of tert-butyl (1-((3-hydroxyphenyl)sulfonyl)piperidin-4-yl)carbamate (200 mg, 0.56 mmol, 1.00 eq.), K2C03 (232 mg, 1.68 mmol, 3.00 eq.), NaI (86 mg, 0.58 mmol, 1.04 eq.) and benzyl (2-bromoethyl)carbamate (288 mg, 1.12 mmol, 2.00 eq.) in DMSO (50.00 mL) was stirred at 60° C. for 12 h. The mixture was quenched with H₂O and then extracted with EtOAc. The organic layer was concentrated and purified by silica gel column chromatography eluting with PE/EtOAc (3:1) to give the title compound (3.98 g, 69.5%) as white solid.

### Step 2: benzyl (2-(3-((4-((8-cyclopentyl-7-oxo-7,8-dihydropyrido[2,3-d]pyrimidin-2-yl)amino)-piperidin-1-yl)sulfonyl)phenoxy)ethyl)carbamate

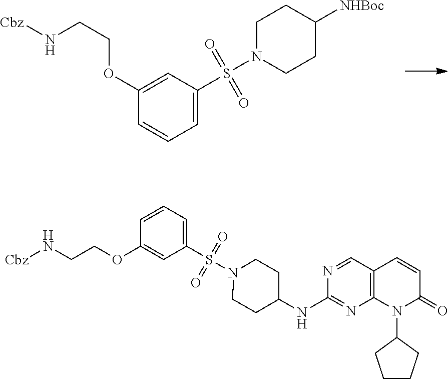 [SMILES: CC(C)(C)OC(=O)NC1CCN(S(=O)(=O)c2cccc(OCCNC(=O)OCc3ccccc3)c2)CC1 ; O=C(NCCOc1cccc(S(=O)(=O)N2CCC(Nc3ncc4ccc(=O)n(C5CCCC5)c4n3)CC2)c1)OCc1ccccc1]

Benzyl (2-(3-((4-aminopiperidin-1-yl)sulfonyl)phenoxy)ethyl)carbamate was converted to the title compound by proceeding analogously as described in Example 7, Steps 5-6. The residue was purified by silica gel column chromatography, eluted with PE/EA (1:2), to afford the title compound (230 mg, 63.7%) as a yellow solid.

### Step 3: 2-((1-((3-(2-aminoethoxy)phenyl)sulfonyl)piperidin-4-yl)amino)-8-cyclopentylpyrido-[2,3-d]pyrimidin-7 (8H)-one

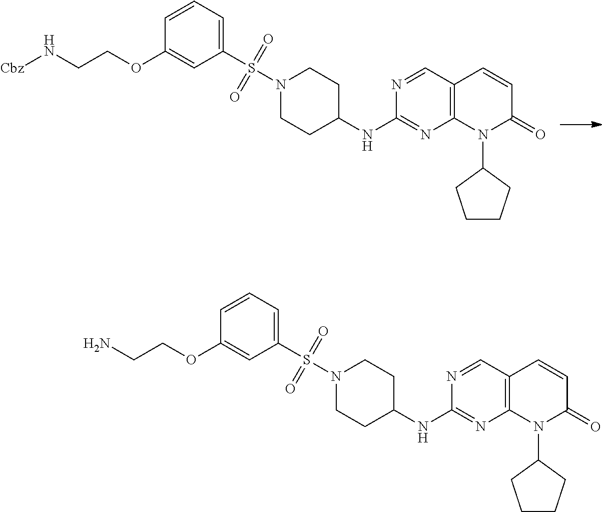 [SMILES: O=C(NCCOc1cccc(S(=O)(=O)N2CCC(Nc3ncc4ccc(=O)n(C5CCCC5)c4n3)CC2)c1)OCc1ccccc1 ; NCCOc1cccc(S(=O)(=O)N2CCC(Nc3ncc4ccc(=O)n(C5CCCC5)c4n3)CC2)c1]

To a stirred solution of benzyl (2-(3-((4-((8-cyclopentyl-7-oxo-7,8-dihydropyrido[2,3-d]pyrimidin-2-yl)amino)piperidin-1-yl)sulfonyl)phenoxy)ethyl)carbamate (230 mg, 0.36 mmol, 1.00 eq.) in EtOH (3.00 mL) and THF (2.00 mL) was added Pd/C (150 mg). The resulting mixture was stirred at RT under H2 for 12 h. The mixture was filtered and concentrated to afford the crude product (182 mg, crude) as a white solid.

### Step 4: 1-(2,6-dioxopiperidin-3-yl)-3-methyl-2-oxo-2,3-dihydro-1H-benzo[d]imidazole-4-carbonitrile

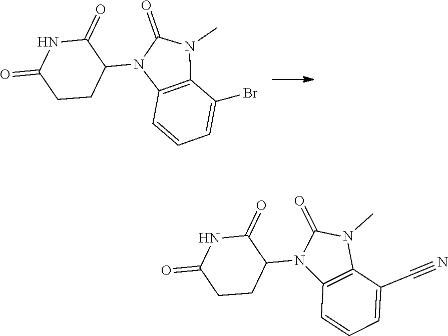 [SMILES: Cn1c(=O)n(C2CCC(=O)NC2=O)c2cccc(Br)c21 ; Cn1c(=O)n(C2CCC(=O)NC2=O)c2cccc(C#N)c21]

A mixture of 3-(4-bromo-3-methyl-2-oxo-2,3-dihydro-1H-benzo[d]imidazol-1-yl)-piperidine-2,6-dione (1.35 g, 4.0 mmol, 1.00 eq.), ZnCN₂ (704 mg, 6.0 mmol, 1.50 eq.) and Pd(PPh₃)₄ (462.0 mg, 0.4 mmol, 0.010 eq.) in DMF (20.0 mL) was stirred at 100° C. under N₂ for 16 h. The resulting mixture was concentrated and the residue was purified by silica gel column chromatography, eluted with DCM/MeOH (20:1), to afford the title compound (1.20 g, 100%) as a yellow solid.

### Step 5: 1-(2,6-dioxopiperidin-3-yl)-3-methyl-2-oxo-2,3-dihydro-1H-benzo[d]imidazole-4-carbaldehyde

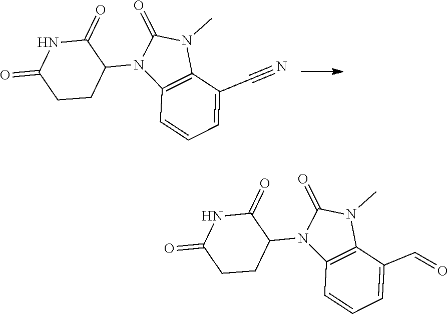 [SMILES: Cn1c(=O)n(C2CCC(=O)NC2=O)c2cccc(C#N)c21 ; Cn1c(=O)n(C2CCC(=O)NC2=O)c2cccc(C=O)c21]

A mixture of 1-(2,6-dioxopiperidin-3-yl)-3-methyl-2-oxo-2,3-dihydro-1H-benzo[d]-imidazole-4-carbonitrile (700 mg, 2.46 mmol, 1.00 eq.), NaH₂PO₂·H₂O (1.80 g, 17.2 mmol, 7.00 eq.) and Raney Ni (1.80 g) in pyridine (20.0 mL), H₂O (10.0 mL) and AcOH (10.0 mL) was stirred for 16 h at 70° C. under nitrogen atmosphere. The resulting mixture was diluted with EtOAc and washed with water, brine, dried over anhydrous Na₂SO₄ and concentrated. The residue was purified by silica gel column chromatography, eluted with DCM/MeOH (80:1), to afford the title compound (160 mg, 22.6%) as a yellow solid.

### Step 6: 3-(4-(((2-(3-((4-((8-cyclopentyl-7-oxo-7,8-dihydropyrido[2,3-d]pyrimidin-2-yl)amino)-piperidin-1-yl)sulfonyl)phenoxy)ethyl)amino)methyl)-3-methyl-2-oxo-2,3-dihydro-1H-benzo[d]imidazol-1-yl)piperidine-2,6-dione

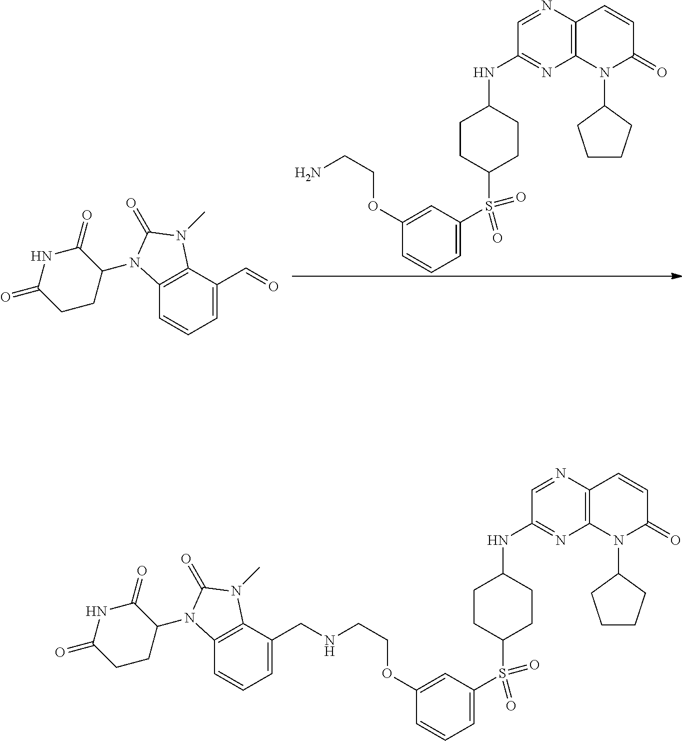 [SMILES: Cn1c(=O)n(C2CCC(=O)NC2=O)c2cccc(C=O)c21 ; NCCOc1cccc(S(=O)(=O)C2CCC(Nc3cnc4ccc(=O)n(C5CCCC5)c4n3)CC2)c1 ; Cn1c(=O)n(C2CCC(=O)NC2=O)c2cccc(CNCCOc3cccc(S(=O)(=O)C4CCC(Nc5cnc6ccc(=O)n(C7CCCC7)c6n5)CC4)c3)c21]

To a solution of 2-((1-((3-(2-aminoethoxy)phenyl)sulfonyl)piperidin-4-yl)amino)-8-cyclopentylpyrido[2,3-d]pyrimidin-7 (8H)-one (100 mg, 0.2 mmol, 1.00 eq.) in THF (2.0 mL) and DMF (2.0 mL) was added 1-(2,6-dioxopiperidin-3-yl)-3-methyl-2-oxo-2,3-dihydro-1H-benzo[d]imidazole-4-carbaldehyde (86 mg, 0.30 mmol, 1.50 eq.) and AcOH (12.0 mg, 0.2 mmol, 1.00 eq.) and the solution was stirred at RT for 1 h. NaBH₃CN (38 mg, 0.60 mmol, 3.00 eq.) was added to this mixture and the mixture was stirred at RT for 3 h. The resulting mixture was concentrated and the residue was purified by silica gel column chromatography, eluted with DCM/MeOH (15:1), to afford the title compound (21 mg, 13.4%) as a white solid. MS (ES, m/z): [M+1]⁺=784.3.

### Example 42

### Synthesis of 3-(5-(4-(3-((4-((8-cyclopentyl-7-oxo-7,8-dihydropyrido[2,3-d]pyrimidin-2-yl)-amino)piperidin-1-yl)sulfonyl)benzyl)piperazin-1-yl)-6-fluoro-1-oxoisoindolin-2-yl)piperidine-2,6-dione

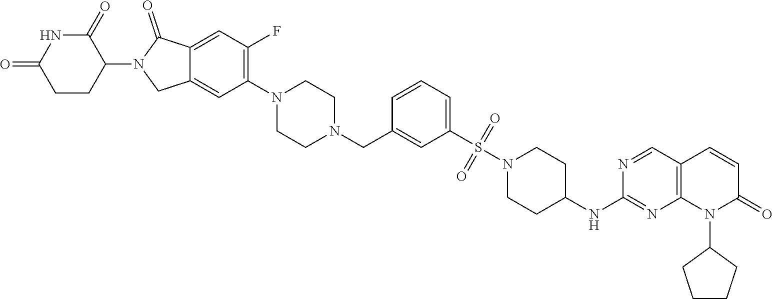 [SMILES: O=C1CCC(N2Cc3cc(N4CCN(Cc5cccc(S(=O)(=O)N6CCC(Nc7ncc8ccc(=O)n(C9CCCC9)c8n7)CC6)c5)CC4)c(F)cc3C2=O)C(=O)N1]

### Step 1: tert-butyl 4-(5-bromo-2-fluoro-4-(methoxycarbonyl)phenyl)piperazine-1-carboxylate

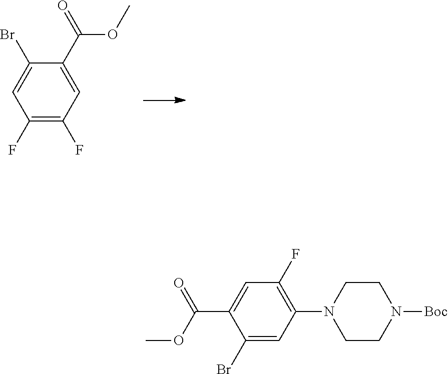 [SMILES: COC(=O)c1cc(F)c(F)cc1Br ; COC(=O)c1cc(F)c(N2CCN(C(=O)OC(C)(C)C)CC2)cc1Br]

To a solution of methyl 2-bromo-4,5-difluorobenzoate (2.00 g, 8.00 mmol, 1.00 eq.) and tert-butyl piperazine-1-carboxylate (2.23 g, 12.00 mmol, 1.50 eq.) in DMA (6.0 mL) was added K₂CO₃ (1.65 g, 12.00 mmol, 1.50 eq.), the mixture was stirred at 80° C. overnight. The mixture was extracted EA and water, the organic layer was washed with brine, dried over Na₂SO₄, concentrated and purified by flash chromatography (PE:EA=3:1) to give the title compound (3.00 g, 91.0%) as a colorless oil.

### Step 2: tert-butyl 4-(5-cyano-2-fluoro-4-(methoxycarbonyl)phenyl)piperazine-1-carboxylate

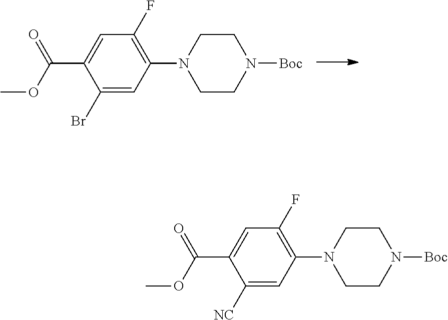 [SMILES: COC(=O)c1cc(F)c(N2CCN(C(=O)OC(C)(C)C)CC2)cc1Br ; COC(=O)c1cc(F)c(N2CCN(C(=O)OC(C)(C)C)CC2)cc1C#N]

A mixture of tert-butyl 4-(5-bromo-2-fluoro-4-(methoxycarbonyl)phenyl)-piperazine-1-carboxylate (1.50 g, 3.60 mmol, 1.00 eq.) and CuCN (484 mg, 5.40 mmol, 1.50 eq.) in DMF (6.0 mL was stirred at 100° C. overnight. The mixture was extracted with EA and NH₃·H₂O. The organic layer was washed with water and brine, dried over Na₂SO₄, concentrated and purified by flash chromatography (PE:EA=3:1) to give the title compound (570 mg, 43.8%) as a white solid.

### Step 3: tert-butyl 4-(2-(2,6-dioxopiperidin-3-yl)-6-fluoro-1-oxoisoindolin-5-yl)piperazine-1-carboxylate

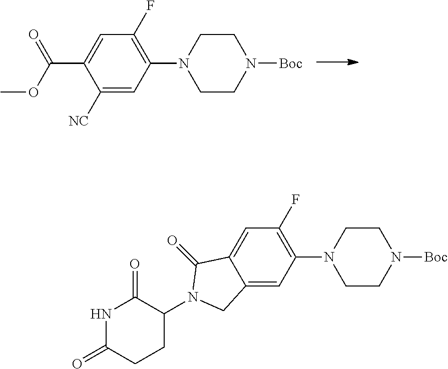 [SMILES: COC(=O)c1cc(F)c(N2CCN(C(=O)OC(C)(C)C)CC2)cc1C#N ; CC(C)(C)OC(=O)N1CCN(c2cc3c(cc2F)C(=O)N(C2CCC(=O)NC2=O)C3)CC1]

tert-butyl 4-(5-Cyano-2-fluoro-4-(methoxycarbonyl)phenyl)piperazine-1-carboxylate was converted to the title compound by proceeding analogously as described in Example 34, Step 6-7.

### Step 4: 3-(6-fluoro-1-oxo-5-(piperazin-1-yl)isoindolin-2-yl)piperidine-2,6-dione

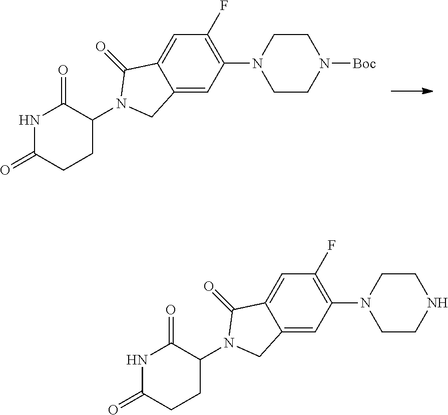 [SMILES: CC(C)(C)OC(=O)N1CCN(c2cc3c(cc2F)C(=O)N(C2CCC(=O)NC2=O)C3)CC1 ; O=C1CCC(N2Cc3cc(N4CCNCC4)c(F)cc3C2=O)C(=O)N1]

To a stirred solution of tert-butyl 4-(2-(2,6-dioxopiperidin-3-yl)-6-fluoro-1-oxoisoindolin-5-yl)piperazine-1-carboxylate (95 mg, 0.21 mmol, 1.00 eq.) in DCM (2.0 mL) was added TFA (0.5 mL) and the mixture was stirred at RT for 2 h. The reaction mixture was concentrated to give the title compound (74 mg, crude) as a yellow oil.

### Step 5: tert-butyl (1-((3-((4-(2-(2,6-dioxopiperidin-3-yl)-6-fluoro-1-oxoisoindolin-5-yl)piperazin-1-yl)methyl)phenyl)sulfonyl)piperidin-4-yl)carbamate

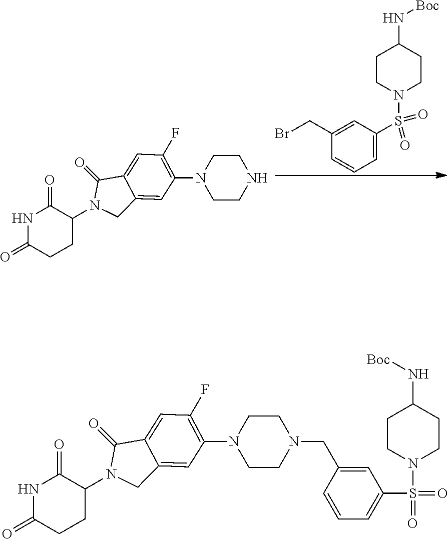 [SMILES: O=C1CCC(N2Cc3cc(N4CCNCC4)c(F)cc3C2=O)C(=O)N1 ; CC(C)(C)OC(=O)NC1CCN(S(=O)(=O)c2cccc(CBr)c2)CC1 ; CC(C)(C)OC(=O)NC1CCN(S(=O)(=O)c2cccc(CN3CCN(c4cc5c(cc4F)C(=O)N(C4CCC(=O)NC4=O)C5)CC3)c2)CC1]

To a solution of 3-(6-fluoro-1-oxo-5-(piperazin-1-yl)isoindolin-2-yl)piperidine-2,6-dione (74 mg, 0.33 mmol, 1.00 eq.) and tert-butyl (1-((3-(bromomethyl)phenyl)sulfonyl)piperidin-4-yl)carbamate (138 mg, 0.32 mmol, 1.50 eq.) in THF (5.0 mL) was added TEA (127 mg, 1.26 mmol, 6.00 eq.) and the mixture was stirred at 55° C. overnight. The mixture was extracted DCM and water. The organic layer was washed with brine, dried over Na₂SO₄, concentrated, and the residue was purified by flash chromatography (DCM:MeOH=20:1) to give the title compound (95 mg, 64.6%) as a yellow solid.

### Step 6: 3-(5-(4-(3-((4-((8-cyclopentyl-7-oxo-7,8-dihydropyrido[2,3-d]pyrimidin-2-yl)amino)-piperidin-1-yl)sulfonyl)benzyl)piperazin-1-yl)-6-fluoro-1-oxoisoindolin-2-yl)piperidine-2,6-dione

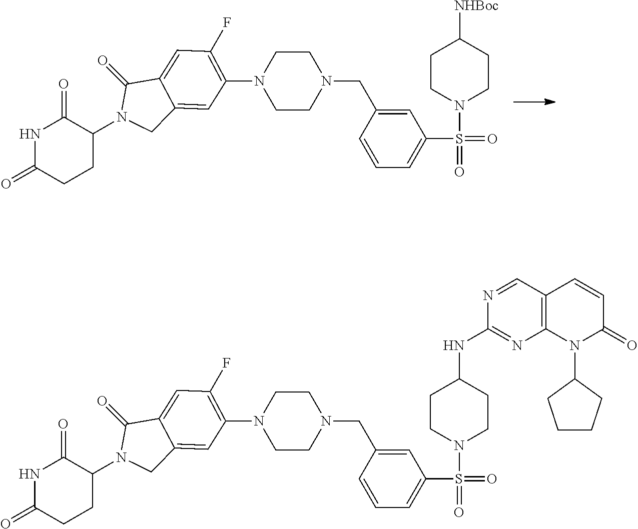 [SMILES: CC(C)(C)OC(=O)NC1CCN(S(=O)(=O)c2cccc(CN3CCN(c4cc5c(cc4F)C(=O)N(C4CCC(=O)NC4=O)C5)CC3)c2)CC1 ; O=C1CCC(N2Cc3cc(N4CCN(Cc5cccc(S(=O)(=O)N6CCC(Nc7ncc8ccc(=O)n(C9CCCC9)c8n7)CC6)c5)CC4)c(F)cc3C2=O)C(=O)N1]

tert-butyl (1-((3-((4-(2-(2,6-Dioxopiperidin-3-yl)-6-fluoro-1-oxoisoindolin-5-yl)piperazin-1-yl)methyl)phenyl)sulfonyl)piperidin-4-yl)carbamate was converted to the title compound by proceeding analogously as described in Example 7, Steps 5-6. MS (ES, m/z): [M+1]⁺=812.4

### Example 43

### Synthesis of 5-(4-(3-((4-((8-cyclopentyl-7-oxo-7,8-dihydropyrido[2,3-d]pyrimidin-2-yl)amino)-piperidin-1-yl)sulfonyl)-5-fluorobenzyl)piperazin-1-yl)-2-(2,6-dioxopiperidin-3-yl)-6-fluoroisoindoline-1,3-dione

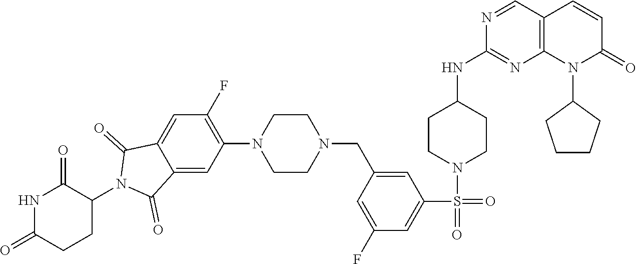 [SMILES: O=C1CCC(N2C(=O)c3cc(F)c(N4CCN(Cc5cc(F)cc(S(=O)(=O)N6CCC(Nc7ncc8ccc(=O)n(C9CCCC9)c8n7)CC6)c5)CC4)cc3C2=O)C(=O)N1]

### Step 1: 3-fluoro-5-methylbenzene-1-sulfonyl chloride

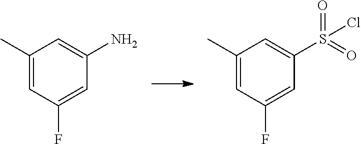

SOCl₂ (1.98 g, 1.2 mL, 6.68 mmol, 4.00 eq.) was added dropwise to water (5.0 mL) at 0° C. and the mixture was stirred at RT overnight. CuCl (23 mg, 0.24 mmol, 0.06 eq.) was added and the mixture was stirred at 0° C. for 15 min to get a solution (Solution A).

To a solution of 3-fluoro-5-methylaniline (500 mg, 4.00 mmol, 1.00 eq.) in HCl (4.0 mL, 40.00 mmol, 10.00 eq. 1OM) was added NaNO₂ (303 mg, 4.40 mmol, 1.10 eq.) in water (1.0 mL) dropwise at 0° C. and the mixture was stirred at 0° C. for 15 min. Solution A was added slowly at 0° C. and the mixture was stirred at 0° C. for 2 h. The mixture was extracted with DCM and water and the organic layer was washed with brine, dried over Na₂SO₄, and concentrated. Purification of the residue by flash chromatography (100% PE) to give the title compound (200 mg, 25%) as a brown oil.

### Step 2: 3-(bromomethyl)-5-fluorobenzene-1-sulfonyl chloride

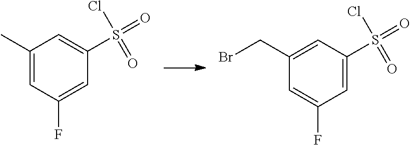

To a solution of 3-fluoro-5-methylbenzene-1-sulfonyl chloride (550 mg, 2.64 mmol, 1.00 eq.) in CCl₄ (10.0 mL) were added NBS (494 mg, 2.78 mmol, 1.05 eq.) and benzoyl peroxide (91 mg, 0.26 mmol, 0.10 eq. 70%) and the mixture was stirred at 80° C. overnight. The mixture was filtered off and the filtrate was concentrated and purified by flash chromatography (PE 100%) to give give the title compound (280 mg, 37.7%) as a yellow oil.

### Step 3: 5-(4-(3-((4-((8-cyclopentyl-7-oxo-7,8-dihydropyrido[2,3-d]pyrimidin-2-yl)amino)-piperidin-1-yl)sulfonyl)-5-fluorobenzyl)piperazin-1-yl)-2-(2,6-dioxopiperidin-3-yl)-6-fluoroisoindoline-1,3-dione

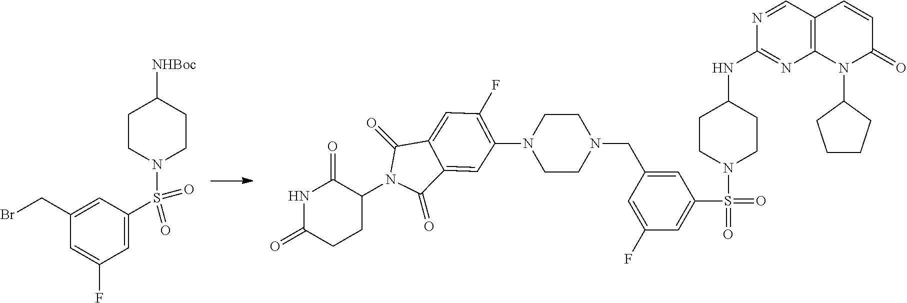 [SMILES: CC(C)(C)OC(=O)NC1CCN(S(=O)(=O)c2cc(F)cc(CBr)c2)CC1 ; O=C1CCC(N2C(=O)c3cc(F)c(N4CCN(Cc5cc(F)cc(S(=O)(=O)N6CCC(Nc7ncc8ccc(=O)n(C9CCCC9)c8n7)CC6)c5)CC4)cc3C2=O)C(=O)N1]

tert-butyl (1-((3-(Bromomethyl)-5-fluorophenyl)sulfonyl)piperidin-4-yl)carbamate was converted to the title compound by proceeding analogously as described in Example 42, Steps 5-6.

MS (ES, m/z): [M+1]⁺=844.5

### Example 44

### Synthesis of 2-(2,6-dioxopiperidin-3-yl)-5-fluoro-6-(4-(3-((4-((8-(2-hydroxy-2-methy-cyclopentyl)-7-oxo-7,8-dihydropyrido[2,3-d]pyrimidin-2-yl)amino)piperidin-1-yl)-sulfonyl)benzyl)piperazin-1-yl)isoindoline-1,3-dione

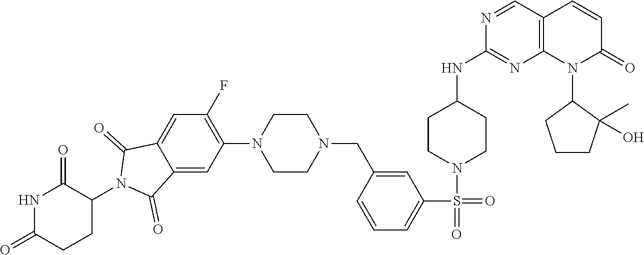 [SMILES: CC1(O)CCCC1n1c(=O)ccc2cnc(NC3CCN(S(=O)(=O)c4cccc(CN5CCN(c6cc7c(cc6F)C(=O)N(C6CCC(=O)NC6=O)C7=O)CC5)c4)CC3)nc21]

### Step 1: 1-methyl-6-oxabicyclo[3.1.0]hexane

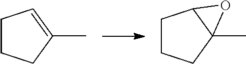

To a solution of 1-methylcyclopent-1-ene (4.92 g, 60.00 mmol, 1.00 eq.) in DCM (50.0 mL) was added m-CPBA (11.40 g, 66.00 mmol, 1.10 eq.) at 0° C. and the mixture was stirred at RT for 16 h. The mixture was quenched with sat. aq. Na₂SO₃ solution and sat. aq. NaHCO₃ solution and extracted with DCM. The combined organic layer was washed with water, dried over anhydrous Na₂SO₄, filtered, and concentrated to afford the title compound (3.74 g, 63.6%) as a yellow oil.

### Step 2: 2-(benzylamino)-1-methylcyclopentanol

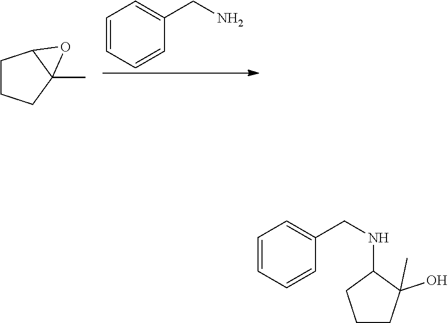

A mixture of 1-methyl-6-oxabicyclo[3.1.0]hexane (3.74 g, 38.10 mmol, 1.00 eq.) and phenylmethanamine (4.08 g, 38.10 mmol, 1.00 eq.) in H₂O (50.0 mL) was stirred at 100° C. for 16 h. The mixture was diluted with water, and then extracted with EtOAc. The combined organic layer was washed with water, dried over anhydrous Na₂SO₄, filtered, and concentrated to afford the title compound (2.50 g, 32.0%) as a yellow solid.

### Step 3: 2-amino-1-methylcyclopentanol

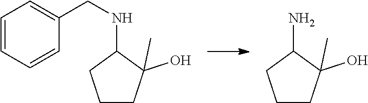

A mixture of 2-(benzylamino)-1-methylcyclopentanol (4.00 g, 7.20 mmol, 1.00 eq.) and Pd(OH)₂ (500 mg) in i-PrOH (40.0 mL) was stirred at 50° C. under H₂ (50 psi) for 16 h. The mixture was filtered and concentrated to afford the title compound (1.40 g, 100%) as a yellow oil.

### Step 4: (4-chloro-2-(methylthio)pyrimidin-5-yl)methanol

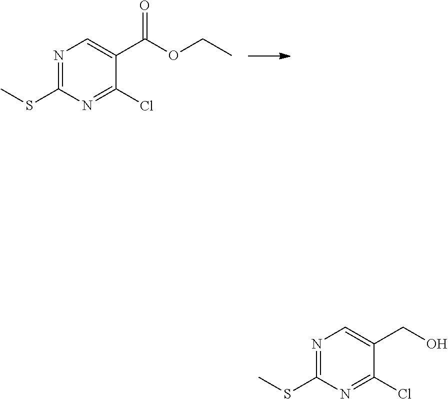

To a solution of ethyl 4-chloro-2-(methylthio)pyrimidine-5-carboxylate (9.30 g, 40.00 mmol, 1.00 eq.) in THF (120.0 mL) was added DIBAL-H (120.0 mL, 120.00 mmol, 3.00 eq.) dropwise slowly at −78° C. under N₂ atmosphere and the mixture was stirred at RT for 16 h. The mixture was quenched with sat. aq. NH₄Cl solution and extracted with EtOAc. The combined organic layer was washed with water, dried over anhydrous Na₂SO₄, filtered, and concentrated.

The residue was purified by silica gel column chromatography, eluted with PE/EtOAc (6:1), to afford the title compound (3.50 g, 46.0%) as a white solid.

### Step 5: 2-((5-(hydroxymethyl)-2-(methylthio)pyrimidin-4-yl)amino)-1-methylcyclopentanol

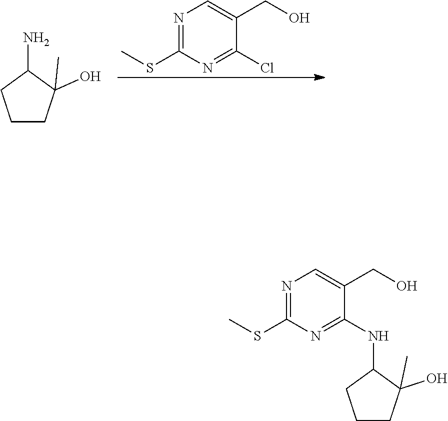

A mixture of 2-amino-1-methylcyclopentanol (920 mg, 8.00 mmol, 1.00 eq.), (4-chloro-2-(methylthio)pyrimidin-5-yl)methanol (1.52 g, 8.00 mmol, 1.00 eq.) and K₂CO₃ (3.31 g, 24.00 mmol, 1.00 eq.) in i-PrOH (20 mL) was stirred at 50° C. for 16 h. The mixture was diluted with water and then extracted with EtOAc. The combined organic layer was washed with water, dried over anhydrous Na₂SO₄, filtered, and concentrated. The residue was purified by silica gel column chromatography, eluted with PE/EtOAc (1:1), to afford the title compound (830 mg, 38.1%) as a yellow solid.

### Step 6: 4-((2-hydroxy-2-methylcyclopentyl)amino)-2-(methylthio)pyrimidine-5-carbaldehyde

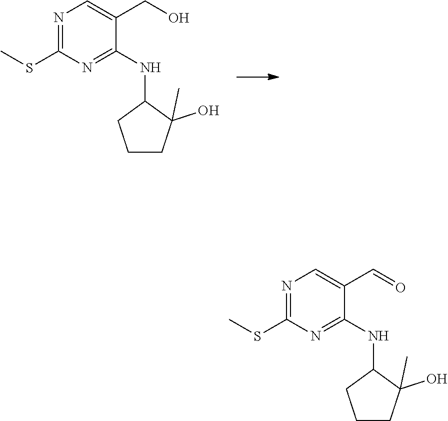 [SMILES: CSc1ncc(CO)c(NC2CCCC2(C)O)n1 ; CSc1ncc(C=O)c(NC2CCCC2(C)O)n1]

A mixture of 2-((5-(hydroxymethyl)-2-(methylthio)pyrimidin-4-yl)amino)-1-methyl-cyclopentanol (820 mg, 3.05 mmol, 1.00 eq.) and MnO₂ (2.65 g, 30.50 mmol, 10.00 eq.) in DCM (40 mL) was stirred at RT for 16 h. The mixture was filtered and concentrated to afford the title compound (700 mg, 86.0%) as a yellow solid.

### Step 7: 8-(2-hydroxy-2-methylcyclopentyl)-2-(methylthio)pyrido[2,3-d pyrimidin-7 (8H)-one

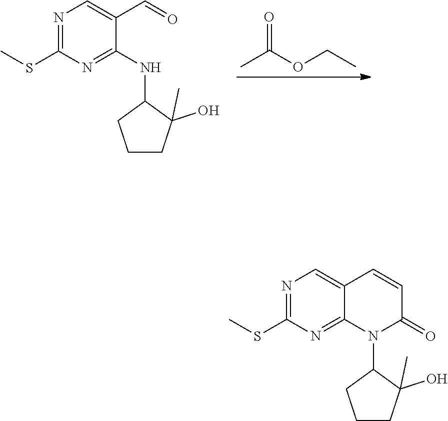

A solution of 4-((2-hydroxy-2-methylcyclopentyl)amino)-2-(methylthio)pyrimidine-5-carbaldehyde (700 mg, 2.62 mmol, 1.00 eq.) and ethyl acetate (692 mg, 7.86 mmol, 3.00 eq.) in THF (20 mL) was cooled to −78° C. under N₂ atmosphere and LiHMDS (13.1 mL, 13.1 mmol, 5.00 eq.) was added by syringe slowly. The mixture was stirred at −78° C. for 6 h, then warm up to RT and stirred for 16 h. The mixture was quenched with sat. aq. NH₄Cl solution and extracted with EtOAc. The combined organic layer was washed with water, dried over anhydrous Na₂SO₄, filtered, and concentrated. The residue was purified by silica gel column chromatography, eluted with PE/EtOAc (4:1), to afford the title compound (400 mg, 52.5%) as a yellow solid.

### Step 8: 8-(2-hydroxy-2-methylcyclopentyl)-2-(methylsulfonyl)pyrido[2,3-d]pyrimidin-7 (8H)-one

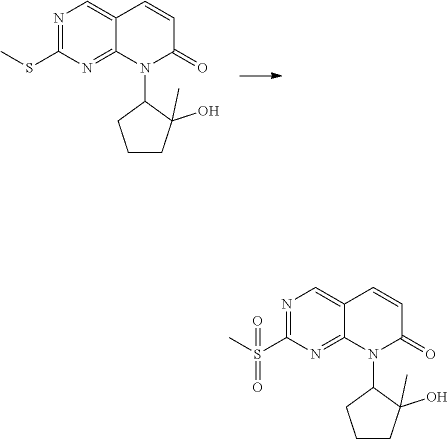 [SMILES: CSc1ncc2ccc(=O)n(C3CCCC3(C)O)c2n1 ; CC1(O)CCCC1n1c(=O)ccc2cnc(S(C)(=O)=O)nc21]

A mixture of 8-(2-hydroxy-2-methylcyclopentyl)-2-(methylthio)pyrido[2,3-d]pyrimidin-7 (8H)-one (100 mg, 0.34 mmol, 1.00 eq.) and Oxone (417.5 mg, 0.68 mmol, 2.00 eq.) in THF (4.0 mL) and H₂O (2.0 mL) was stirred at RT for 16 h. The mixture diluted with water, and extracted with EtOAc. The combined organic layer was washed with water, dried over anhydrous Na₂SO₄, filtered, and concentrated. The residue was purified by silica gel column chromatography, eluted with DCM/MeOH (60:1), to afford the title compound (110 mg, 100%) as a yellow oil.

### Step 9: 2-(2,6-dioxopiperidin-3-yl)-5-fluoro-6-(4-(3-((4-((8-(2-hydroxy-2-methylcyclopentyl)-7-oxo-7,8-dihydropyrido[2,3-d]pyrimidin-2-yl)amino)piperidin-1-yl)sulfonyl)benzyl)piperazin-1-1 isoindoline-1,3-dione

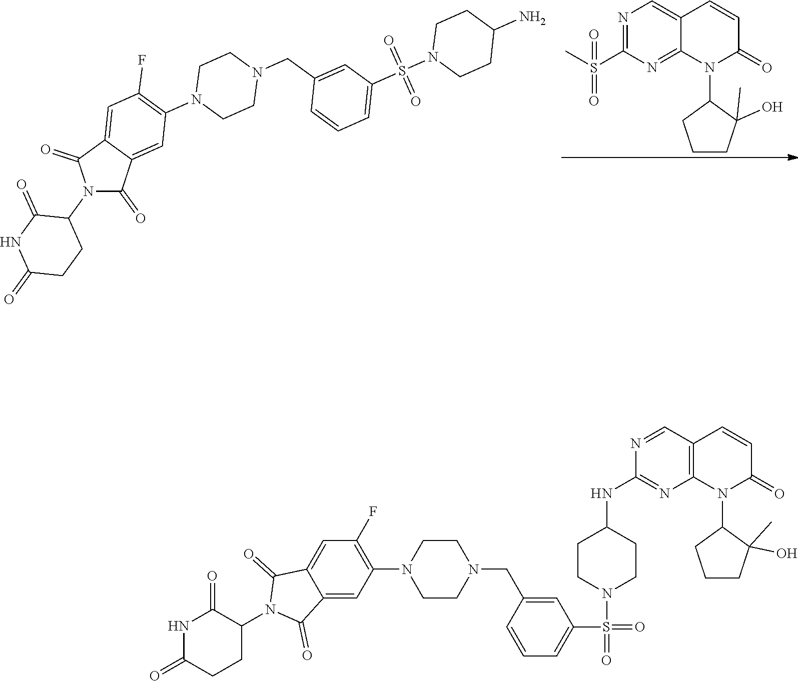 [SMILES: NC1CCN(S(=O)(=O)c2cccc(CN3CCN(c4cc5c(cc4F)C(=O)N(C4CCC(=O)NC4=O)C5=O)CC3)c2)CC1 ; CC1(O)CCCC1n1c(=O)ccc2cnc(S(C)(=O)=O)nc21 ; CC1(O)CCCC1n1c(=O)ccc2cnc(NC3CCN(S(=O)(=O)c4cccc(CN5CCN(c6cc7c(cc6F)C(=O)N(C6CCC(=O)NC6=O)C7=O)CC5)c4)CC3)nc21]

A solution of 5-(4-(3-((4-aminopiperidin-1-yl)sulfonyl)benzyl)piperazin-1-yl)-2-(2,6-dioxopiperidin-3-yl)-6-fluoroisoindoline-1,3-dione (65 mg, 0.10 mmol, 1.00 eq.), 8-(2-hydroxy-2-methylcyclopentyl)-2-(methylsulfonyl)pyrido[2,3-d]pyrimidin-7 (8H)-one (38.8 mg, 0.12 mmol, 1.20 eq.) and DIEA (38.7 mg, 0.30 mmol, 3.00 eq.) in DMSO (2.0 mL) was stirred at 65° C. under N₂ atmosphere for 16 h. The mixture was cooled to RT, diluted with water, and extracted with EtOAc. The combined organic layer was washed with water, dried over anhydrous Na₂SO₄, filtered, and then concentrated. The residue was purified by TLC, eluted with DCM/MeOH (15:1), to afford the title compound (25 mg, 29.2%) as a yellow solid. MS (ES, m/z): [M−18+1]⁺=838.4.

### Example 45

### Synthesis of 5-(4-(3-((4-((8-cyclopentyl-6-(difluoromethyl)-7-oxo-7,8-dihydropyrido[2,3-d]-pyrimidin-2-yl)amino)piperidin-1-yl)sulfonyl)benzyl)piperazin-1-yl)-2-(2,6-dioxopiperidin-3-yl)-6-fluoroisoindoline-1,3-dione

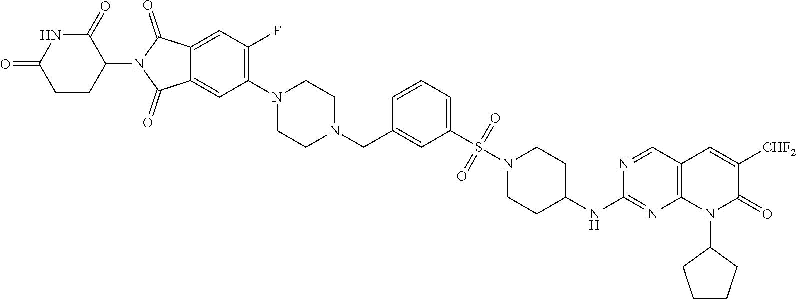 [SMILES: O=C1CCC(N2C(=O)c3cc(F)c(N4CCN(Cc5cccc(S(=O)(=O)N6CCC(Nc7ncc8cc(C(F)F)c(=O)n(C9CCCC9)c8n7)CC6)c5)CC4)cc3C2=O)C(=O)N1]

### Step 1: tert-butyl 4-(2-(2,6-dioxopiperidin-3-yl)-6-fluoro-1,3-dioxoisoindolin-5-yl)piperazine-1-carboxylate

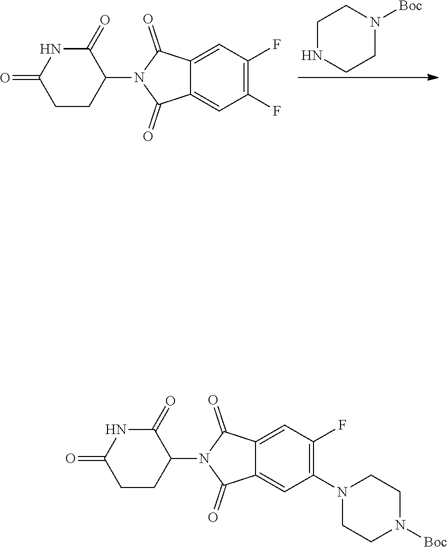

To a solution of tert-butyl piperazine-1-carboxylate (950 mg, 5.10 mmol, 1.00 eq.) and 2-(2,6-dioxopiperidin-3-yl)-5,6-difluoroisoindoline-1,3-dione (1.50 g, 5.10 mmol, 1.00 eq.) in NMP (15.0 mL) was added DIEA (1.97 g, 15.30 mmol, 3.00 eq.) and the mixture was stirred at 110° C. overnight. The mixture was extracted EA and water. The organic layer was washed with brine, dried over Na₂SO₄, concentrated and purified by flash chromatography (PE:EA=1:2) to give the title compound (2.20 g, 94%) as a yellow solid.

### Step 2: 2-(2,6-dioxopiperidin-3-yl)-5-fluoro-6-(piperazin-1-yl)isoindoline-1,3-dione

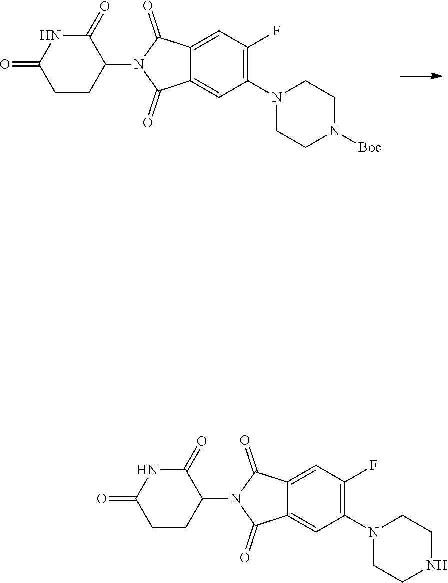 [SMILES: CC(C)(C)OC(=O)N1CCN(c2cc3c(cc2F)C(=O)N(C2CCC(=O)NC2=O)C3=O)CC1 ; O=C1CCC(N2C(=O)c3cc(F)c(N4CCNCC4)cc3C2=O)C(=O)N1]

To a stirred solution of tert-butyl 4-(2-(2,6-dioxopiperidin-3-yl)-6-fluoro-1,3-dioxoisoindolin-5-yl)piperazine-1-carboxylate (800 mg, 1.74 mmol, 1.00 eq.) in DCM (4.0 mL) was added TFA (1.0 mL) and the mixture was stirred at RT for 2 h. The reaction mixture was concentrated to give the title compound (626 mg, crude) as a yellow oil.

### Step 3: 3-(bromomethyl)benzenesulfonyl chloride

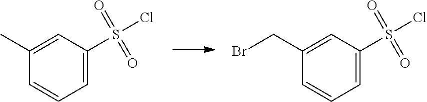

To a stirred solution of 3-methylbenzenesulfonyl chloride (8.00 g, 41.96 mol, 1.00 eq.) in CCl₄ (80.00 mL) was added NBS (8.22 g, 46.16 mol, 1.10 eq.) and benzoyl peroxide (1.46 g, 4.20 mol, 0.01 eq.). The solution was stirred at 80° C. for 12 h. The solution was filtered and the filtrate was concentrated to give crude product (9.01 g, crude) as white oil, which was used to next step without further purification.

### Step 4: tert-butyl (1-((3-(bromomethyl)phenyl)sulfonyl)piperidin-4-yl)carbamate

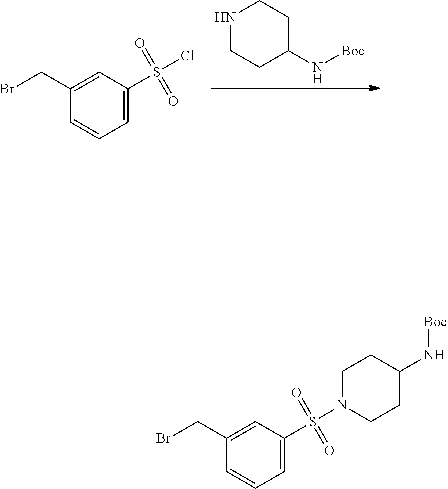

To a stirred solution of 3-(bromomethyl)benzenesulfonyl chloride (3.79 g, 18.95 mol, 0.90 eq.) in THF (40.00 mL) was added TEA (4.25 g, 42.10 mmol, 2.00 eq.). tert-butyl piperidin-4-ylcarbamate (5.64 g, 21.05 mol, 1.00 eq.) in THF (20.00 mL) was added at 0° C. The resulting mixture was stirred at RT for 12 h, quenched with H₂O and then extracted with DCM. The combined organic layer was washed with water, dried over anhydrous Na₂SO₄, filtered, and concentrated. The residue was purified by silica gel column chromatography, eluted with PE/EA (3:1), to afford the title compound (5.53 g, 60.8%) as white solid.

### Step 5: tert-butyl (1-((3-((4-(2-(2,6-dioxopiperidin-3-yl)-6-fluoro-1,3-dioxoisoindolin-5-yl)piperazin-1-yl)methyl)phenyl)sulfonyl)piperidin-4-yl)carbamate

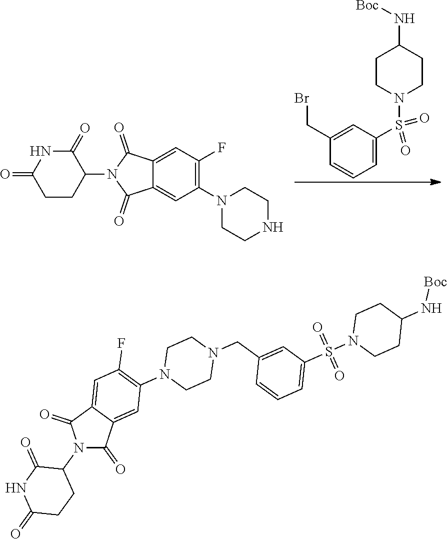 [SMILES: O=C1CCC(N2C(=O)c3cc(F)c(N4CCNCC4)cc3C2=O)C(=O)N1 ; CC(C)(C)OC(=O)NC1CCN(S(=O)(=O)c2cccc(CBr)c2)CC1 ; CC(C)(C)OC(=O)NC1CCN(S(=O)(=O)c2cccc(CN3CCN(c4cc5c(cc4F)C(=O)N(C4CCC(=O)NC4=O)C5=O)CC3)c2)CC1]

To a solution of 2-(2,6-dioxopiperidin-3-yl)-5-fluoro-6-(piperazin-1-yl)isoindoline-1,3-dione (509 mg, 1.41 mmol, 1.00 eq.) and tert-butyl (1-((3-(bromomethyl)phenyl)sulfonyl)-piperidin-4-yl)carbamate (916 mg, 2.12 mmol, 1.50 eq.) in THF (10.0 mL) was added TEA (854 mg, 8.46 mmol, 6.00 eq.) and the mixture was stirred at 55° C. overnight. The mixture was extracted DCM and water. The organic layer was washed with brine, dried over Na₂SO₄, filtered and concentrated. The residue was purified by flash chromatography (DCM:MeOH=20:1) to give the title compound (545 mg, 51.4%) as a yellow solid.

### Step 6: 8-cyclopentyl-6-(difluoromethyl)-2-(methylsulfonyl)pyrido[2,3-d]pyrimidin-7 (8H)-one

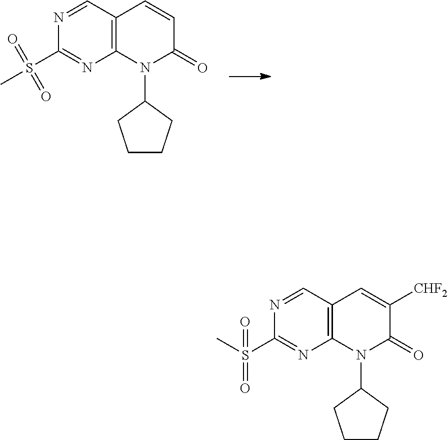 [SMILES: CS(=O)(=O)c1ncc2ccc(=O)n(C3CCCC3)c2n1 ; CS(=O)(=O)c1ncc2cc(C(F)F)c(=O)n(C3CCCC3)c2n1]

To a solution of 8-cyclopentyl-2-(methylsulfonyl)pyrido[2,3-d]pyrimidin-7 (8H)-one (500.0 mg, 1.70 mmol, 1.00 eq.) in DMSO (20.00 mL) was added TFA (194.0 mg, 1.70 mmol, 1.00 eq.), FeCl₂ (107.00 mg, 0.85 mmol, 0.50 eq.), zinc difluoro methanesulfinate (1.50 g, 5.10 mmol, 3.00 eq.) and tert-butyl hydroperoxide (70% in H₂O, 218.60 mg, 1.70 mmol, 1.00 eq.), the mixture was stirred at RT for 16 h. Then another batch of tert-butyl hydroperoxide (70% in H₂O, 218.60 mg, 1.70 mmol, 1.00 eq.) was added to this mixture and stirred at RT for 8 h. Then a third batch of tert-butyl hydroperoxide (70% in H₂O, 218.60 mg, 1.70 mmol, 1.00 eq.) was added to this mixture and stirred at RT for 16 h. The mixture was diluted with water and extracted with DCM. The combined organic layer was washed with water, aqueous Na₂CO₃, water and brine. The organic layer was concentrated and the residue was purified by silica gel column chromatography, eluted with EtOAc/PE (1:10), to afford the title compound (220 mg, 37.7%) as a yellow solid.

### Step 7: 5-(4-(3-((4-((8-cyclopentyl-6-(difluoromethyl)-7-oxo-7,8-dihydropyrido[2,3-d]pyrimidin-2-yl)amino)piperidin-1-yl)sulfonyl)benzyl)piperazin-1-yl)-2-(2,6-dioxopiperidin-3-yl)-6-fluoro-isoindoline-1,3-dione

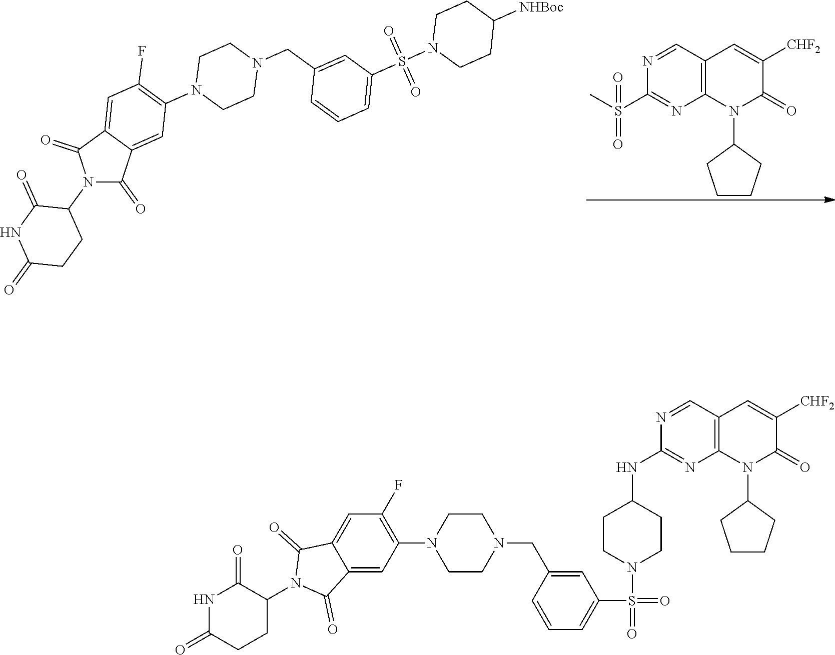 [SMILES: CC(C)(C)OC(=O)NC1CCN(S(=O)(=O)c2cccc(CN3CCN(c4cc5c(cc4F)C(=O)N(C4CCC(=O)NC4=O)C5=O)CC3)c2)CC1 ; CS(=O)(=O)c1ncc2cc(C(F)F)c(=O)n(C3CCCC3)c2n1 ; O=C1CCC(N2C(=O)c3cc(F)c(N4CCN(Cc5cccc(S(=O)(=O)N6CCC(Nc7ncc8cc(C(F)F)c(=O)n(C9CCCC9)c8n7)CC6)c5)CC4)cc3C2=O)C(=O)N1]

tert-butyl (1-((3-((4-(2-(2,6-Dioxopiperidin-3-yl)-6-fluoro-1,3-dioxo-isoindolin-5-yl)piperazin-1-yl)methyl)phenyl)sulfonyl)piperidin-4-yl)carbamate was converted to the title compound by proceeding analogously as described in Example 7, Steps 5-6. MS (ES, m/z): [M+1]⁺=876.4

### Example 46

### Synthesis of 8-cyclopentyl-2-((1-((3-((4-(2-(2,6-dioxopiperidin-3-yl)-6-fluoro-1,3-dioxo-isoindolin-5-yl)piperazin-1-yl)methyl)phenyl)sulfonyl)piperidin-4-yl)amino)-7-oxo-7,8-dihydropyrido[2,3-d]pyrimidine-6-carbonitrile

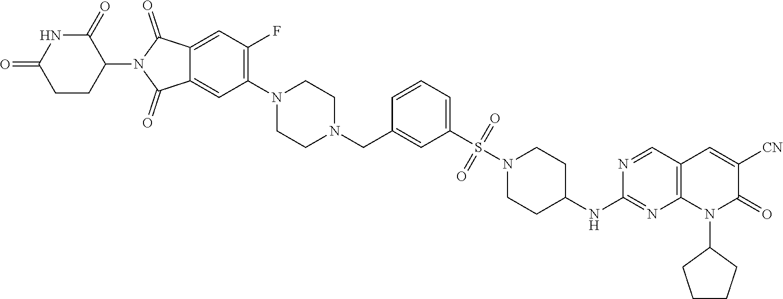 [SMILES: N#Cc1cc2cnc(NC3CCN(S(=O)(=O)c4cccc(CN5CCN(c6cc7c(cc6F)C(=O)N(C6CCC(=O)NC6=O)C7=O)CC5)c4)CC3)nc2n(C2CCCC2)c1=O]

### Step 1: 8-cyclopentyl-2-(methylthio)-7-oxo-7,8-dihydropyrido[2,3-d]pyrimidine-6-carbonitrile

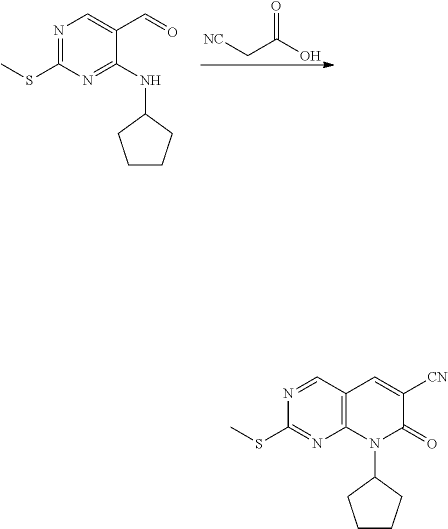

A solution of 4-(cyclopentylamino)-2-(methylthio)pyrimidine-5-carbaldehyde (360 mg, 1.50 mmol, 1.00 eq.), 2-cyanoacetic acid (153 mg, 1.80 mmol, 1.20 eq.) and benzylamine (16 mg, 0.15 mmol, 0.10 eq.) in acetic acid (5.0 mL) was stirred at 100° C. for 6 h. The mixture was cooled to RT, diluted with water, and then extracted with EtOAc. The combined organic layer was washed with water, dried over anhydrous Na₂SO₄, filtered, and concentrated. The residue was purified by silica gel column chromatography, eluted with PE/EtOAc (6:1), to afford the title compound (210 mg, 38.2%) as a yellow solid.

### Step 2: 8-cyclopentyl-2-(methylsulfonyl)-7-oxo-7,8-dihydropyrido[2,3-d]pyrimidine-6-carbonitrile

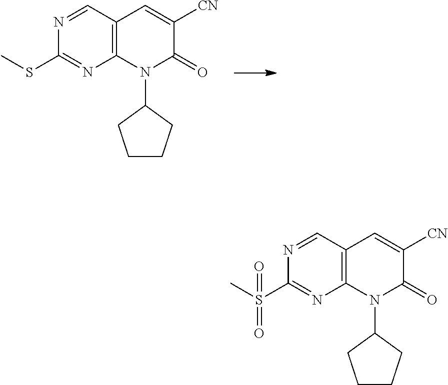 [SMILES: CSc1ncc2cc(C#N)c(=O)n(C3CCCC3)c2n1 ; CS(=O)(=O)c1ncc2cc(C#N)c(=O)n(C3CCCC3)c2n1]

A mixture of 8-cyclopentyl-2-(methylthio)-7-oxo-7,8-dihydropyrido[2,3-d]pyrimidine-6-carbonitrile (200 mg, 0.70 mmol, 1.00 eq.) and Oxone (860 mg, 1.40 mmol, 2.00 eq.) in THF (6.0 mL) and H₂O (3.0 mL) was stirred at RT for 16 h. The mixture diluted with water and extracted with EtOAc. The combined organic layer was washed with water, dried over anhydrous Na₂SO₄, filtered, and concentrated. The residue was purified by silica gel column chromatography, eluted with DCM/MeOH (70:1), to afford the title compound (100 mg, 45.0%) as a yellow solid.

### Step 3: 8-cyclopentyl-2-((1-((3-((4-(2-(2,6-dioxopiperidin-3-yl)-6-fluoro-1,3-dioxoisoindolin-5-yl)piperazin-1-yl)methyl)phenyl)sulfonyl)piperidin-4-yl)amino)-7-oxo-7,8-dihydropyrido[2,3-d]pyrimidine-6-carbonitrile

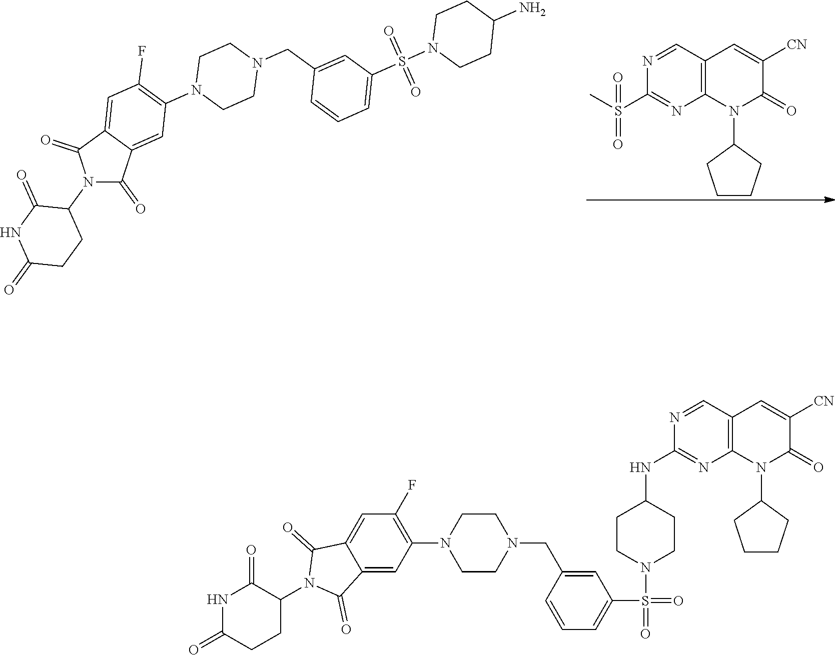 [SMILES: NC1CCN(S(=O)(=O)c2cccc(CN3CCN(c4cc5c(cc4F)C(=O)N(C4CCC(=O)NC4=O)C5=O)CC3)c2)CC1 ; CS(=O)(=O)c1ncc2cc(C#N)c(=O)n(C3CCCC3)c2n1 ; N#Cc1cc2cnc(NC3CCN(S(=O)(=O)c4cccc(CN5CCN(c6cc7c(cc6F)C(=O)N(C6CCC(=O)NC6=O)C7=O)CC5)c4)CC3)nc2n(C2CCCC2)c1=O]

A solution of 5-(4-(3-((4-aminopiperidin-1-yl)sulfonyl)benzyl)piperazin-1-yl)-2-(2,6-dioxopiperidin-3-yl)-6-fluoroisoindoline-1,3-dione (65 mg, 0.10 mmol, 1.00 eq.), 8-cyclopentyl-2-(methylsulfonyl)-7-oxo-7,8-dihydropyrido[2,3-d]pyrimidine-6-carbonitrile (38 mg, 0.12 mmol, 1.20 eq.) and DIEA (38.7 mg, 0.30 mmol, 3.00 eq.) in DMSO (2.0 mL) was stirred at 65° C. under N₂ atmosphere for 16 h. The mixture was cooled to RT, diluted with water, and then extracted with EtOAc. The combined organic layer was washed with water, dried over anhydrous Na₂SO₄, filtered, and then concentrated. The residue was purified by TLC, eluted with DCM/MeOH (15:1), to afford the title compound (14 mg, 16.4%) as a yellow solid. MS (ES, m/z): [M+1]⁺=851.4.

### Example 47

### Synthesis of 5-(3-(3-((4-((8-cyclopentyl-7-oxo-7,8-dihydropyrido[2,3-d]pyrimidin-2-yl)amino)-piperidin-1-yl)sulfonyl)benzyl)-3,8-diazabicyclo[3.2.1]octan-8-yl)-2-(2,6-dioxopiperidin-3-yl)-6-fluoroisoindoline-1,3-dione

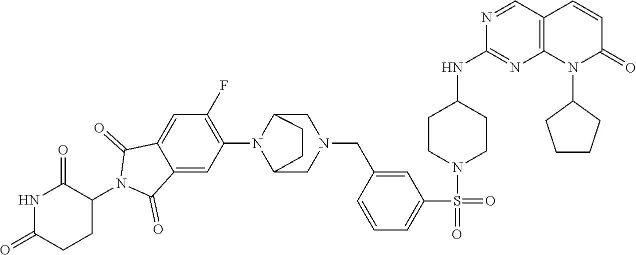 [SMILES: O=C1CCC(N2C(=O)c3cc(F)c(N4C5CCC4CN(Cc4cccc(S(=O)(=O)N6CCC(Nc7ncc8ccc(=O)n(C9CCCC9)c8n7)CC6)c4)C5)cc3C2=O)C(=O)N1]

### Step 1: tert-butyl 8-(2-(2,6-dioxopiperidin-3-yl)-6-fluoro-1,3-dioxoisoindolin-5-yl)-3,8-di-azabicyclo[3.2.1]octane-3-carboxylate

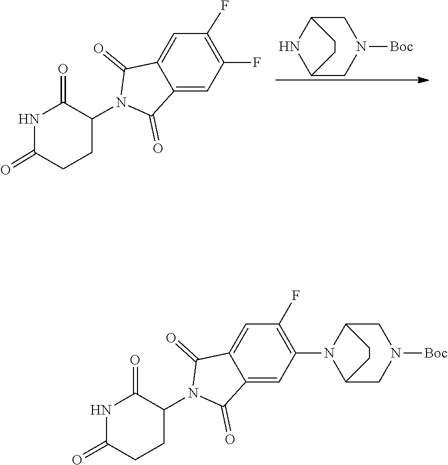

To a solution of 2-(2,6-dioxopiperidin-3-yl)-5,6-difluoroisoindoline-1,3-dione (200 mg, 0.68 mmol, 1.00 eq.) in NMP (3.0 mL) was added tert-butyl 3,8-diazabicyclo[3.2.1]octane-3-carboxylate (144 mg, 0.68 mmol, 1.00 eq.) and DIEA (263 mg, 2.04 mmol, 3.00 eq.). The reaction mixture was stirred at 110° C. for overnight. The reaction mixture was extracted with DCM and purified by silica gel column chromatography eluting with PE/EtOAc (1:1) to give title compound (300 mg, 90.9%) as yellow solid.

### Step 2: tert-butyl (1-((3-((8-(2-(2,6-dioxopiperidin-3-yl)-6-fluoro-1,3-dioxoisoindolin-5-yl)-3,8-diazabicyclo[3.2.1]octan-3-yl)methyl)phenyl)sulfonyl)piperidin-4-yl)carbamate

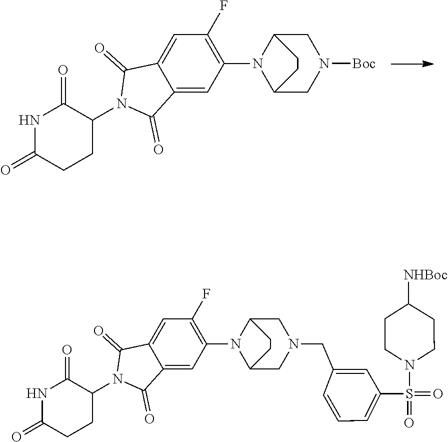 [SMILES: CC(C)(C)OC(=O)N1CC2CCC(C1)N2c1cc2c(cc1F)C(=O)N(C1CCC(=O)NC1=O)C2=O ; CC(C)(C)OC(=O)NC1CCN(S(=O)(=O)c2cccc(CN3CC4CCC(C3)N4c3cc4c(cc3F)C(=O)N(C3CCC(=O)NC3=O)C4=O)c2)CC1]

tert-butyl 8-(2-(2,6-Dioxopiperidin-3-yl)-6-fluoro-1,3-dioxoisoindolin-5-yl)-3,8-diazabicyclo[3.2.1]octane-3-carboxylate was converted to above compound proceeding analogously as described in Example 45, Step 2-5.

### Step 3: 5-(3-(3-((4-((8-cyclopentyl-7-oxo-7,8-dihydropyrido[2,3-d]pyrimidin-2-yl)amino)-piperidin-1-yl)sulfonyl)benzyl)-3,8-diazabicyclo[3.2.1]octan-8-yl)-2-(2,6-dioxopiperidin-3-yl)-6-fluoroisoindoline-1,3-dione

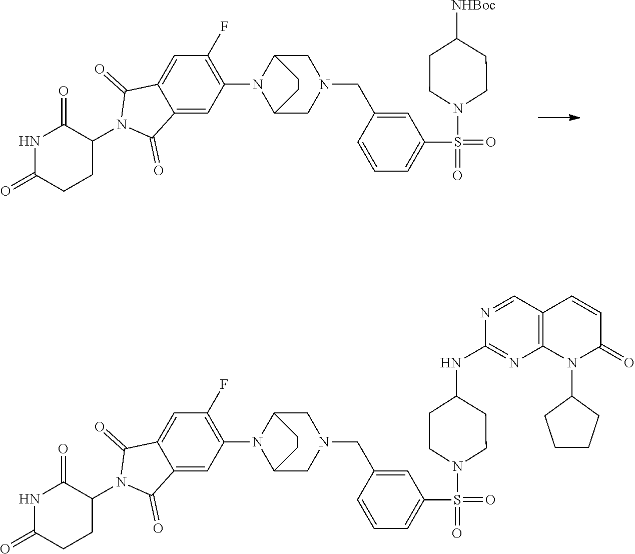 [SMILES: CC(C)(C)OC(=O)NC1CCN(S(=O)(=O)c2cccc(CN3CC4CCC(C3)N4c3cc4c(cc3F)C(=O)N(C3CCC(=O)NC3=O)C4=O)c2)CC1 ; O=C1CCC(N2C(=O)c3cc(F)c(N4C5CCC4CN(Cc4cccc(S(=O)(=O)N6CCC(Nc7ncc8ccc(=O)n(C9CCCC9)c8n7)CC6)c4)C5)cc3C2=O)C(=O)N1]

tert-butyl (1-((3-((8-(2-(2,6-Dioxopiperidin-3-yl)-6-fluoro-1,3-dioxoisoindolin-5-yl)-3,8-diazabicyclo[3.2.1]octan-3-yl)methyl)phenyl)sulfonyl)piperidin-4-yl)carbamate was converted to the title compound by proceeding analogously as described in Example 7, Step 5-6. MS (ES, m/z): [M+1]⁺=852.4.

### Example 48

### Synthesis of 5-(4-(3-((4-((8-cyclopentyl-7-oxo-7,8-dihydropyrido[2,3-d]pyrimidin-2-yl)amino)-piperidin-1-yl)sulfonyl)benzyl)piperazin-1-yl)-2-(2,6-dioxopiperidin-3-yl)-6-fluoroisoindoline-1,3-dione

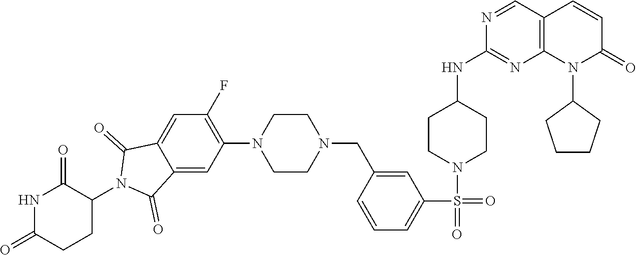 [SMILES: O=C1CCC(N2C(=O)c3cc(F)c(N4CCN(Cc5cccc(S(=O)(=O)N6CCC(Nc7ncc8ccc(=O)n(C9CCCC9)c8n7)CC6)c5)CC4)cc3C2=O)C(=O)N1]

### Step 1: 5-(4-(3-((4-((8-cyclopentyl-7-oxo-7,8-dihydropyrido[2,3-d]pyrimidin-2-yl)amino)-piperidin-1-yl)sulfonyl)benzyl)piperazin-1-yl)-2-(2,6-dioxopiperidin-3-yl)-6-fluoroisoindoline-1,3-dione

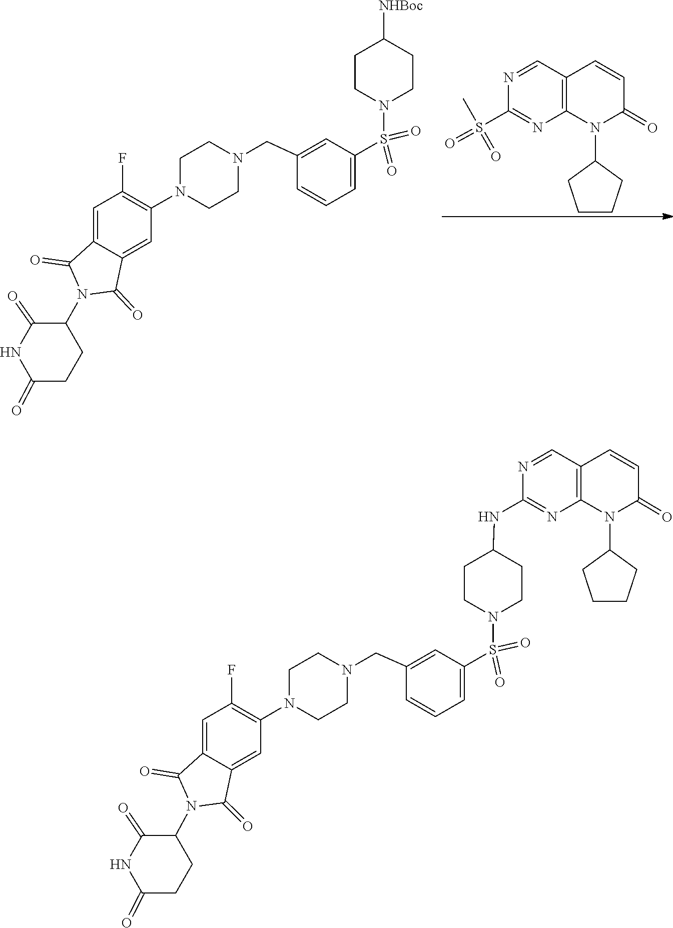

tert-butyl (1-((3-((4-(2-(2,6-Dioxopiperidin-3-yl)-6-fluoro-1,3-dioxoisoindolin-5-yl)piperazin-1-yl)methyl)phenyl)sulfonyl)piperidin-4-yl)carbamate was converted to above compound using similar procedure as described in Example 7, Step 5-6. MS (ES, m/z): [M+1]⁺=826.4.

The compound of Example 49 was prepared by proceeding analogously as described in Example 47.

| | | | |
|---|---|---|---|
| Ex- am- ple 49 | 5-(8-(3-((4- ((8- cyclopentyl- 7-oxo-7,8- dihydro- pyrido [2,3-d]- pyrimidin- 2-yl)- amino) piperidin- 1-yl)- sulfonyl) benzyl)-3,8- diazabicyclo [3.2.1] octan-3- yl)-2-(2,6- dioxo- piperidin- 3-yl)-6- fluoroiso- indoline- 1,3-dione | 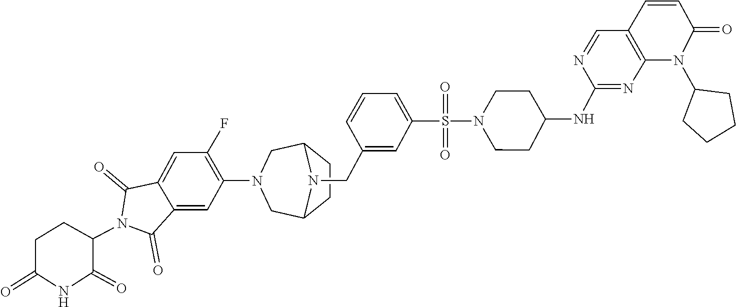 [SMILES: O=C1CCC(N2C(=O)c3cc(F)c(N4CC5CCC(C4)N5Cc4cccc(S(=O)(=O)N5CCC(Nc6ncc7ccc(=O)n(C8CCCC8)c7n6)CC5)c4)cc3C2=O)C(=O)N1] | [M + 1]⁺ = 852.3 |

### Example 50

### Synthesis of 3-(5-(4-(3-((4-((8-cyclopentyl-7-oxo-7,8-dihydropyrido[2,3-d]pyrimidin-2-yl)-amino)piperidin-1-yl)sulfonyl)benzyl)piperazin-1-yl)-1-oxoisoindolin-2-yl)piperidine-2,6-dione

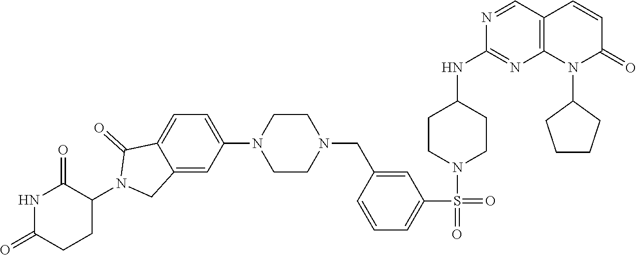 [SMILES: O=C1CCC(N2Cc3cc(N4CCN(Cc5cccc(S(=O)(=O)N6CCC(Nc7ncc8ccc(=O)n(C9CCCC9)c8n7)CC6)c5)CC4)ccc3C2=O)C(=O)N1]

### Step 1: tert-butyl 4-(3-cyano-4-(methoxycarbonyl)phenyl)piperazine-1-carboxylate

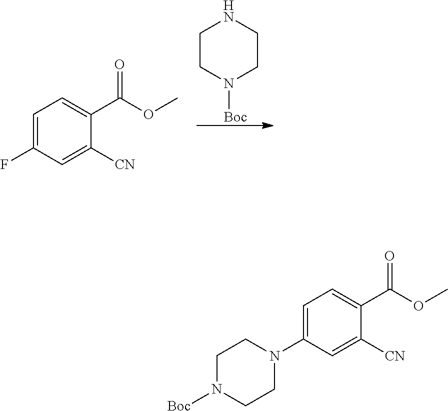

To a stirred solution of methyl 2-cyano-4-fluorobenzoate (10.00 g, 55.80 mmol, 1.00 eq.) in DMSO (150.0 mL) was added tert-butyl piperazine-1-carboxylate (11.40 g, 61.38 mmol, 1.10 eq.) and DIEA (34.70 g, 268.96 mmol, 4.80 eq.). The resulting mixture was stirred at 110° C. for 12 h. The mixture was extracted with EtOAc washed with brine, concentrated and purified by silica gel column chromatography eluting with PE/EtOAc (3:1) to give the title compound (16.60 g, 86%) as yellow solid.

### Step 2: tert-butyl 4-(3-formyl-4-(methoxycarbonyl)phenyl)piperazine-1-carboxylate

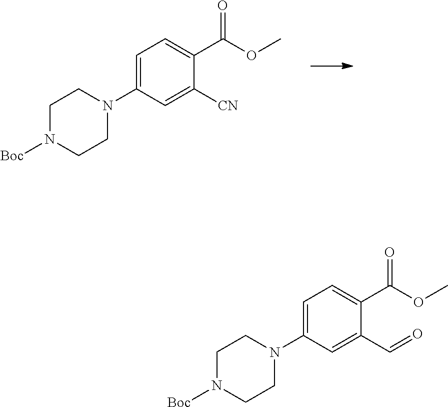 [SMILES: COC(=O)c1ccc(N2CCN(C(=O)OC(C)(C)C)CC2)cc1C#N ; COC(=O)c1ccc(N2CCN(C(=O)OC(C)(C)C)CC2)cc1C=O]

To a stirred solution of tert-butyl 4-(3-cyano-4-(methoxycarbonyl)phenyl)piperazine-1-carboxylate (8.00 g, 23.20 mmol, 1.00 eq.) in pyridine:H₂O:AcOH=2:1:1 (80.0 mL) was added NaH₂PO₂·H₂O (5.20 g, 48.70 mmol, 2.10 eq.) and Raney-Ni (5.10 g). The resulting mixture was stirred at 70° C. for 12 h. The mixture was adjusted pH=7˜8 with aq·NaHCO₃, filtered, and extracted with EtOAc. The organic layer was washed with brine, concentrated and the residue was purified by silica gel column chromatography eluting with PE/EtOAc (3:1) to give the title compound (4.50 g, 55.6%) as yellow solid.

### Step 3: tert-butyl 4-(2-(2,6-dioxopiperidin-3-yl)-1-oxoisoindolin-5-yl)piperazine-1-carboxylate

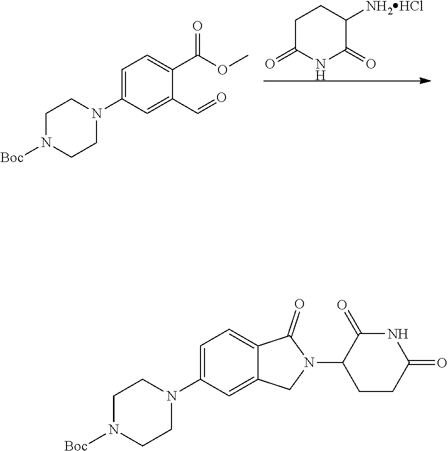

To a stirred solution of 3-aminopiperidine-2,6-dione hydrochloride (2.60 g, 15.50 mmol, 1.20 eq.) in DCM (50.0 mL) were added DIEA (4.03 g, 31.22 mmol, 2.42 eq.), AcOH (10.63 g, 188.76 mmol, 13.78 eq.) and tert-butyl 4-(3-formyl-4-(methoxycarbonyl)phenyl)piperazine-1-carboxylate (4.50 g, 12.90 mmol, 1.00 eq.) at RT. The reaction mixture was stirred at 35° C. for 4 h and then NaBH(OAc)3 (8.20 g, 38.70 mmol, 3.00 eq.) was added at RT. The reaction mixture was stirred at 40° C. for 12 h and was extracted with EtOAc. The organic layer was washed with brine, concentrated, and the residue was purified by silica gel column chromatography eluting with PE/EtOAc (1:2) to give the title compound (2.00 g, 36.4%) as white solid.

### Step 4: 3-(1-oxo-5-(piperazin-1-yl)isoindolin-2-yl)piperidine-2,6-dione

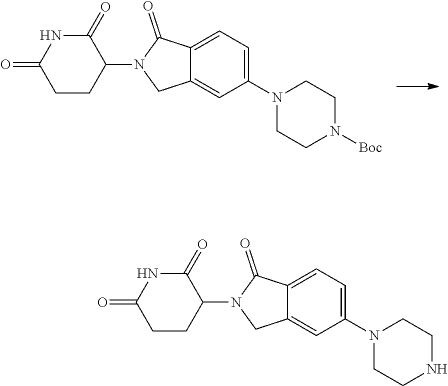 [SMILES: CC(C)(C)OC(=O)N1CCN(c2ccc3c(c2)CN(C2CCC(=O)NC2=O)C3=O)CC1 ; O=C1CCC(N2Cc3cc(N4CCNCC4)ccc3C2=O)C(=O)N1]

To a solution of tert-butyl 4-(2-(2,6-dioxopiperidin-3-yl)-1-oxoisoindolin-5-yl)piperazine-1-carboxylate (72 mg, 0.17 mmol, 1.00 eq.) in DCM (4.0 mL) was added TFA (1.0 mL). The resulting mixture was stirred at RT for 2 h and then concentrated to give the title compound (55 mg, 100%) as yellow oil.

### Step 5: tert-butyl (1-((3-((4-(2-(2,6-dioxopiperidin-3-yl)-1-oxoisoindolin-5-yl)piperazin-1-yl)-methyl)phenyl)sulfonyl)piperidin-4-yl)carbamate

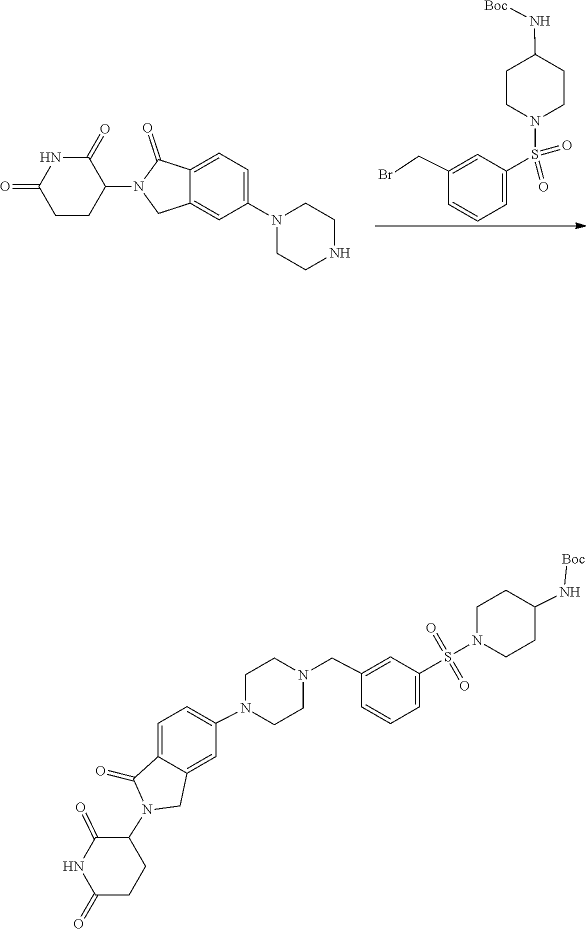 [SMILES: O=C1CCC(N2Cc3cc(N4CCNCC4)ccc3C2=O)C(=O)N1 ; CC(C)(C)OC(=O)NC1CCN(S(=O)(=O)c2cccc(CBr)c2)CC1 ; CC(C)(C)OC(=O)NC1CCN(S(=O)(=O)c2cccc(CN3CCN(c4ccc5c(c4)CN(C4CCC(=O)NC4=O)C5=O)CC3)c2)CC1]

To a stirred solution of 3-(1-oxo-5-(piperazin-1-yl)isoindolin-2-yl)piperidine-2,6-dione (55 mg, 0.17 mmol, 1.00 eq.) in THF (2.0 mL) were added TEA (52 mg, 0.51 mmol, 3.00 eq.) and tert-butyl (1-((3-(bromomethyl)phenyl)sulfonyl)piperidin-4-yl)carbamate (95 mg, 0.22 mmol, 1.30 eq.). The reaction mixture was stirred at 55° C. overnight. The reaction mixture was concentrated and purified by silica gel column chromatography eluting with DCM/MeOH (20:1) to give the title compound (490 mg, crude) as a yellow solid.

### Step 6: 3-(5-(4-(3-((4-((8-cyclopentyl-7-oxo-7,8-dihydropyrido[2,3-d]pyrimidin-2-yl)amino)-piperidin-1-yl)sulfonyl)benzyl)piperazin-1-yl)-1-oxoisoindolin-2-yl)piperidine-2,6-dione

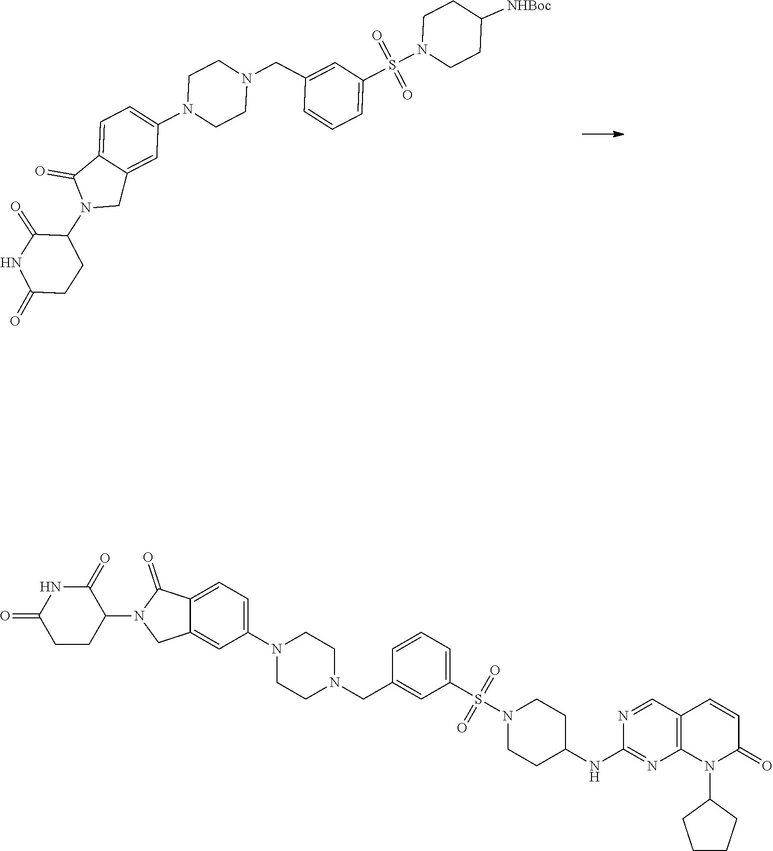 [SMILES: CC(C)(C)OC(=O)NC1CCN(S(=O)(=O)c2cccc(CN3CCN(c4ccc5c(c4)CN(C4CCC(=O)NC4=O)C5=O)CC3)c2)CC1 ; O=C1CCC(N2Cc3cc(N4CCN(Cc5cccc(S(=O)(=O)N6CCC(Nc7ncc8ccc(=O)n(C9CCCC9)c8n7)CC6)c5)CC4)ccc3C2=O)C(=O)N1]

tert-Butyl (1-((3-((4-(2-(2,6-Dioxopiperidin-3-yl)-1-oxoisoindolin-5-yl)piperazin-1-yl)methyl)phenyl)sulfonyl)piperidin-4-yl)carbamate was converted to the title compound using similar procedure as described in Example 7, Step 5-6. MS (ES, m/z): [M+1]⁺=794.5.

Compound of Examples 51-56 were prepared by proceeding analogously as described in Example 50.

| | | | |
|---|---|---|---|
| Example 51 | 3-(5-(5-(3-((4-((8- cyclopentyl-7-oxo-7,8- dihydropyrido[2,3- d]pyrimidin-2- yl)amino)piperidin-1- yl)sulfonyl)-benzyl)-2,5- diazabicyclo[2.2.2]octan- 2-yl)-1-oxoisoindolin- 2-yl)piperidine-2,6- dione | 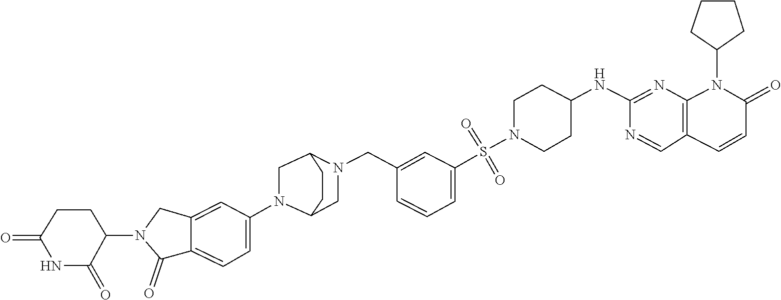 [SMILES: O=C1CCC(N2Cc3cc(N4CC5CCC4CN5Cc4cccc(S(=O)(=O)N5CCC(Nc6ncc7ccc(=O)n(C8CCCC8)c7n6)CC5)c4)ccc3C2=O)C(=O)N1] | [M + 1]⁺ = 820.3 |
| Example 52 | 3-(5-(3-(3-((4-((8- cyclopentyl-7-oxo-7,8- dihydro-pyrido[2,3- d]pyrimidin-2- yl)amino)piperidin-1- yl)sulfonyl)-benzyl)-3,8- diazabicyclo[3.2.1]octan- 8-yl)-1-oxoisoindolin- 2-yl)piperidine-2,6- dione | 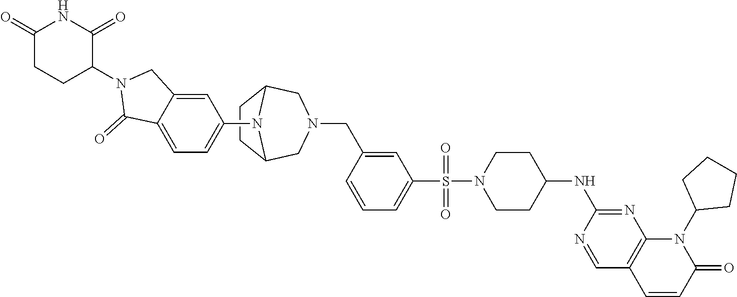 [SMILES: O=C1CCC(N2Cc3cc(N4C5CCC4CN(Cc4cccc(S(=O)(=O)N6CCC(Nc7ncc8ccc(=O)n(C9CCCC9)c8n7)CC6)c4)C5)ccc3C2=O)C(=O)N1] | [M + 1]⁺ = 820.3 |
| Example 53 | 3-(5-(5-(3-((4-((8- cyclopentyl-7-oxo-7,8- dihydro-pyrido[2,3-d]- pyrimidin-2-yl)- amino)piperidin-1- yl)sulfonyl)benzyl)-2,5- diazabicyclo- [2.2.1]heptan-2-yl)-1- oxoiso-indolin-2-yl)- piperidine-2,6-dione | 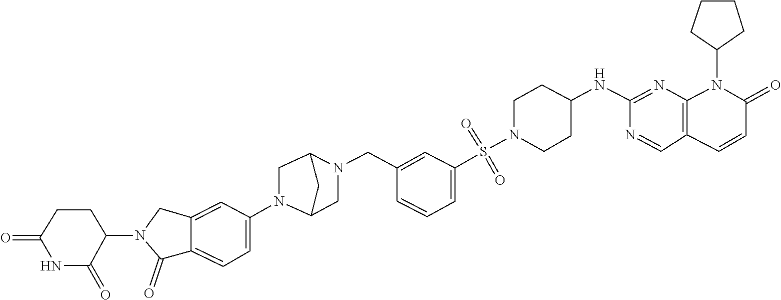 [SMILES: O=C1CCC(N2Cc3cc(N4CC5CC4CN5Cc4cccc(S(=O)(=O)N5CCC(Nc6ncc7ccc(=O)n(C8CCCC8)c7n6)CC5)c4)ccc3C2=O)C(=O)N1] | [M + 1]⁺ = 806.3 |
| Example 54 | 3-(5-(8-(3-((4-((8- cyclopentyl-7-oxo-7,8- dihydro-pyrido[2,3-d]- pyrimidin-2- yl)amino)piperidin-1- yl)sulfonyl)-benzyl)-3,8- diazabicyclo[3.2.1]octan- 3-yl)-1-oxoisoindolin- 2-yl)piperidine-2,6- dione | 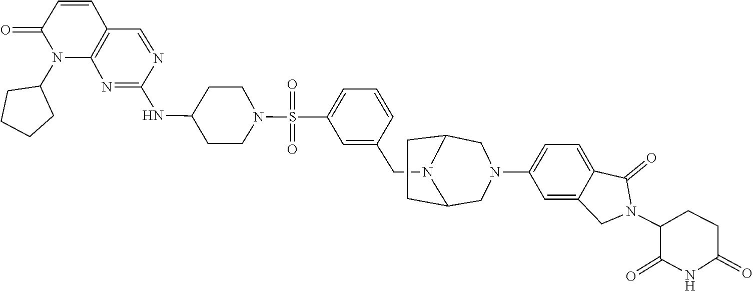 [SMILES: O=C1CCC(N2Cc3cc(N4CC5CCC(C4)N5Cc4cccc(S(=O)(=O)N5CCC(Nc6ncc7ccc(=O)n(C8CCCC8)c7n6)CC5)c4)ccc3C2=O)C(=O)N1] | [M + 1]⁺ = 820.3 |
| Example 55 | 3-(5-(4-(3-((4-((8-(2- hydroxy-2- methylcyclopentyl)-7- oxo-7,8- dihydropyrido[2,3- d]pyrimidin-2- yl)amino)piperidin-1 - yl)sulfonyl)- benzyl)piperazin-1-yl)- 1-oxoiso-indolin-2-yl)- piperidine-2,6-dione | 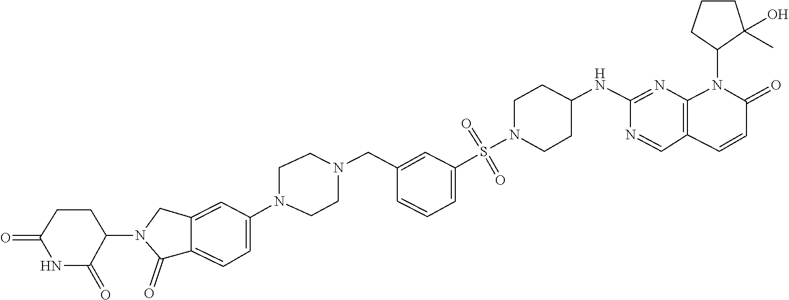 [SMILES: CC1(O)CCCC1n1c(=O)ccc2cnc(NC3CCN(S(=O)(=O)c4cccc(CN5CCN(c6ccc7c(c6)CN(C6CCC(=O)NC6=O)C7=O)CC5)c4)CC3)nc21] | [M + 1]⁺ = 824.3 |
| Example 56 | 3-(5-(4-(3-((4-((8- cyclopentyl-6- (difluoromethyl)-7-oxo- 7,8-dihydropyrido[2,3- d]pyrimidin-2- yl)amino)piperidin-1- yl)sulfonyl)- benzyl)piperazin-1-yl)- 1-oxoiso-indolin-2-yl)- piperidine-2,6-dione | 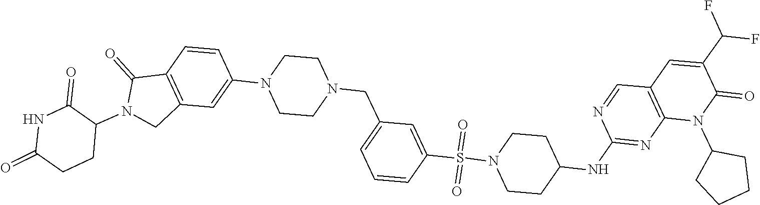 [SMILES: O=C1CCC(N2Cc3cc(N4CCN(Cc5cccc(S(=O)(=O)N6CCC(Nc7ncc8cc(C(F)F)c(=O)n(C9CCCC9)c8n7)CC6)c5)CC4)ccc3C2=O)C(=O)N1] | [M + 1]⁺ = 844.3 |

### Example 57

### Synthesis of 3-(5-(2-(3-((4-((8-cyclopentyl-7-oxo-7,8-dihydropyrido[2,3-d]pyrimidin-2-yl)-amino)piperidin-1-yl)sulfonyl)benzyl)-2,7-diazaspiro[3.5]nonan-7-yl)-1-oxoisoindolin-2-yl)-piperidine-2,6-dione

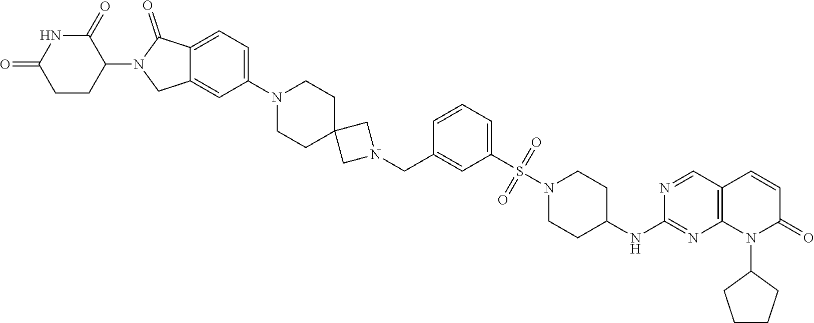 [SMILES: O=C1CCC(N2Cc3cc(N4CCC5(CC4)CN(Cc4cccc(S(=O)(=O)N6CCC(Nc7ncc8ccc(=O)n(C9CCCC9)c8n7)CC6)c4)C5)ccc3C2=O)C(=O)N1]

### Step 1: tert-butyl 7-(3-cyano-4-(methoxycarbonyl)phenyl)-2,7-diazaspiro[3.5]nonane-2-carboxylate

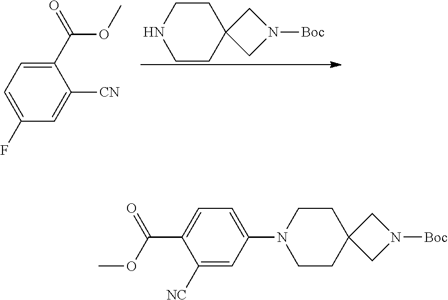

A solution of methyl 2-cyano-4-fluorobenzoate (1.00 g, 5.58 mmol, 1.00 eq.) and tert-butyl 2,7-diazaspiro[3.5]nonane-2-carboxylate (1.39 g, 6.14 mmol, 1.10 eq.) in DMSO (10.0 mL) was added DIEA (719 mg, 16.74 mmol, 3.00 eq.) and the mixture was stirred at 110° C. overnight. The mixture was extracted EA and water and the organic layer was washed with brine, dried over Na₂SO₄, and concentrated The residue was purified by flash chromatography (PE:EA=3:1) to give the title compound (2.00 g, 93.4%) as a white solid.

### Step 2: tert-butyl 7-(2-(2,6-dioxopiperidin-3-yl)-1-oxoisoindolin-5-yl)-2,7-diazaspiro[3.5]nonane-2-carboxylate

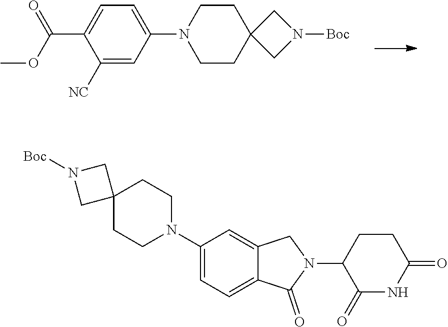 [SMILES: COC(=O)c1ccc(N2CCC3(CC2)CN(C(=O)OC(C)(C)C)C3)cc1C#N ; CC(C)(C)OC(=O)N1CC2(CCN(c3ccc4c(c3)CN(C3CCC(=O)NC3=O)C4=O)CC2)C1]

tert-butyl 7-(3-Cyano-4-(methoxycarbonyl)phenyl)-2,7-diazaspiro[3.5]nonane-2-carboxylate was converted to the title compound by proceeding analogously as described in Example 34, Step 6-7.

### Step 3: 3-(1-oxo-5-(2,7-diazaspiro[3.5]nonan-7-yl)isoindolin-2-yl)piperidine-2,6-dione

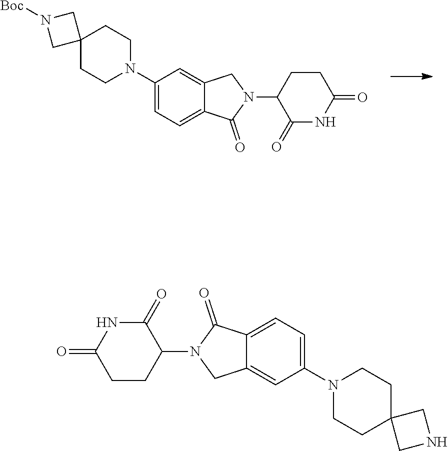 [SMILES: CC(C)(C)OC(=O)N1CC2(CCN(c3ccc4c(c3)CN(C3CCC(=O)NC3=O)C4=O)CC2)C1 ; O=C1CCC(N2Cc3cc(N4CCC5(CC4)CNC5)ccc3C2=O)C(=O)N1]

To a stirred solution of tert-butyl 7-(2-(2,6-dioxopiperidin-3-yl)-1-oxoisoindolin-5-yl)-2,7-diazaspiro[3.5]nonane-2-carboxylate (220 mg, 0.32 mmol, 1.00 eq.) in DCM (2.0 mL) was added TFA (0.5 mL) and the mixture was stirred at RT for 2 h. The reaction mixture was concentrated to give title compound (173 mg, crude) as a yellow oil.

### Step 4: tert-butyl (1-((3-((7-(2-(2,6-dioxopiperidin-3-yl)-1-oxoisoindolin-5-yl)-2,7-diazaspiro-[3.5]nonan-2-yl)methyl)phenyl)sulfonyl)piperidin-4-yl)carbamate

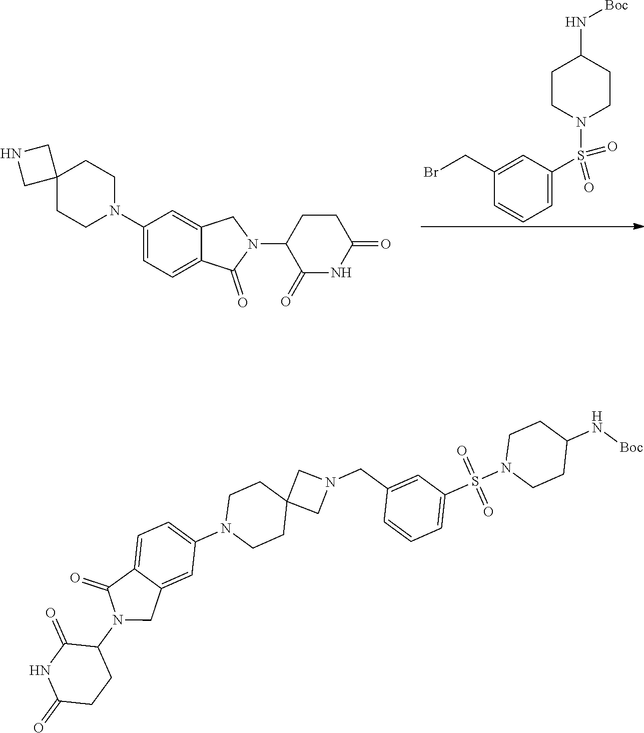 [SMILES: O=C1CCC(N2Cc3cc(N4CCC5(CC4)CNC5)ccc3C2=O)C(=O)N1 ; CC(C)(C)OC(=O)NC1CCN(S(=O)(=O)c2cccc(CBr)c2)CC1 ; CC(C)(C)OC(=O)NC1CCN(S(=O)(=O)c2cccc(CN3CC4(CCN(c5ccc6c(c5)CN(C5CCC(=O)NC5=O)C6=O)CC4)C3)c2)CC1]

To a solution of 3-(1-oxo-5-(2,7-diazaspiro[3.5]nonan-7-yl)isoindolin-2-yl)piperidine-2,6-dione (173 mg, 0.47 mmol, 1.00 eq.) and tert-butyl (1-((3-(bromomethyl)phenyl)sulfonyl)-piperidin-4-yl)carbamate (264 mg, 0.61 mmol, 1.30 eq.) in THF (5.0 mL) was added TEA (285 mg, 2.82 mmol, 6.00 eq.) and the mixture was stirred at 55° C. overnight. The mixture was extracted DCM and water. The organic layer was washed with brine, dried over Na₂SO₄, concentrated, and the residue was purified by flash chromatography (DCM:MeOH=20:1) to give the title compound (40 mg, 11.8%) as a yellow solid.

### Step 5: 3-(5-(2-(3-((4-((8-cyclopentyl-7-oxo-7,8-dihydropyrido[2,3-d]pyrimidin-2-yl)amino)-piperidin-1-yl)sulfonyl)benzyl)-2,7-diazaspiro[3.5]nonan-7-yl)-1-oxoisoindolin-2-yl)piperidine-2,6-dione

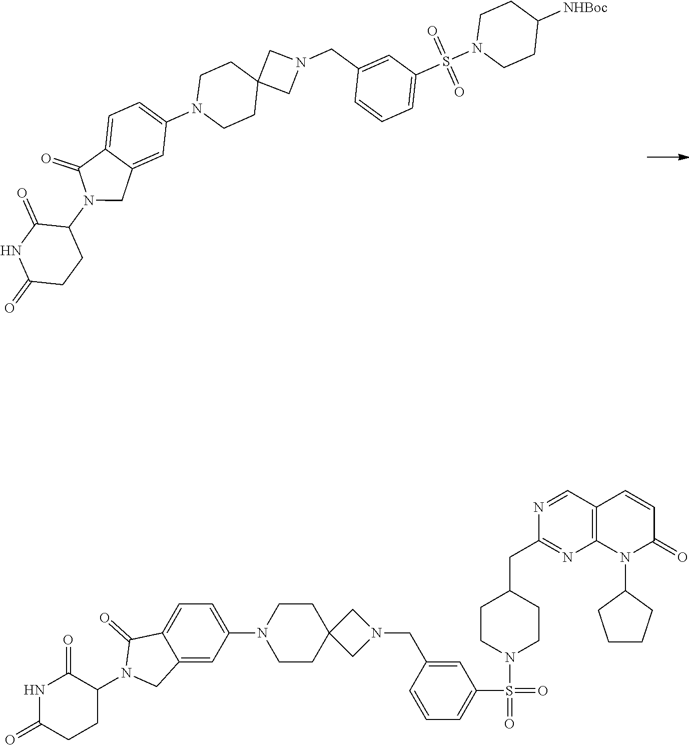 [SMILES: CC(C)(C)OC(=O)NC1CCN(S(=O)(=O)c2cccc(CN3CC4(CCN(c5ccc6c(c5)CN(C5CCC(=O)NC5=O)C6=O)CC4)C3)c2)CC1 ; O=C1CCC(N2Cc3cc(N4CCC5(CC4)CN(Cc4cccc(S(=O)(=O)N6CCC(Cc7ncc8ccc(=O)n(C9CCCC9)c8n7)CC6)c4)C5)ccc3C2=O)C(=O)N1]

tert-butyl (1-3-((7-(2-(2,6-Dioxopiperidin-3-yl)-1-oxoisoindolin-5-yl)-2,7-diazaspiro[3.5]nonan-2-yl)methyl)phenyl)sulfonyl)piperidin-4-yl)carbamate was converged to the title compound by proceeding analogously as described in Example 7, Steps 5-6. MS (ES, m/z): [M+1]⁺=834.5

Compound of Example 58-59 were prepared by proceeding analogously as described in Example 57.

| | | | |
|---|---|---|---|
| Example 58 | 3-(5-(7-(3-((4-((8- cyclopentyl-7-oxo-7,8- dihydropyrido[2,3- d]pyrimidin-2- yl)amino)piperidin-1- yl)sulfonyl)benzyl)-2,7- diazaspiro[3.5]nonan-2- yl)-1-oxoisoindolin-2- yl)piperidine-2,6-dione | 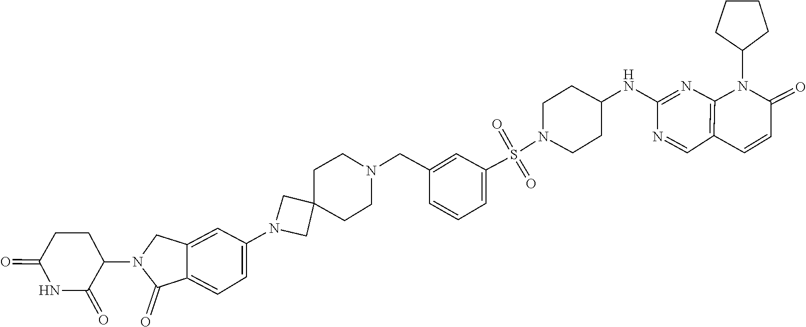 [SMILES: O=C1CCC(N2Cc3cc(N4CC5(CCN(Cc6cccc(S(=O)(=O)N7CCC(Nc8ncc9ccc(=O)n(C%10CCCC%10)c9n8)CC7)c6)CC5)C4)ccc3C2=O)C(=O)N1] | [M + 1]⁺ = 834.4 |
| Example 59 | 3-(5-(9-(3-((4-((8- cyclopentyl-7-oxo-7,8- dihydropyrido[2,3- d]pyrimidin-2- yl)amino)piperidin-1- yl)sulfonyl)benzyl)-3,9- diazaspiro[5.5]undecan- 3-yl)-1-oxoisoindolin-2- yl)piperidine-2,6-dione | 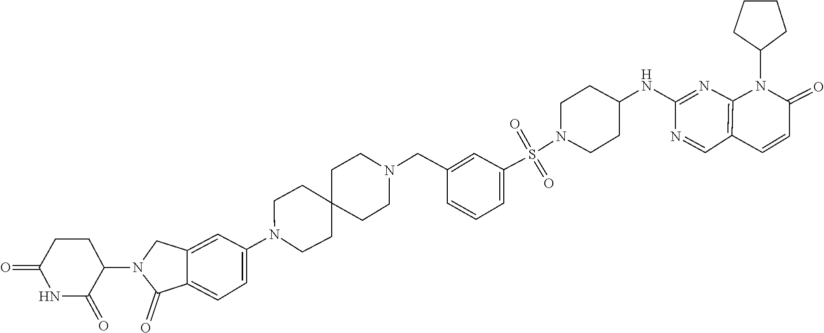 [SMILES: O=C1CCC(N2Cc3cc(N4CCC5(CCN(Cc6cccc(S(=O)(=O)N7CCC(Nc8ncc9ccc(=O)n(C%10CCCC%10)c9n8)CC7)c6)CC5)CC4)ccc3C2=O)C(=O)N1] | [M + 1]⁺ = 862.3 |
| Example 60 | 3-(5-(6-(3-((4-((8- cyclopentyl-7-oxo-7,8- dihydro-pyrido[2,3-d]- pyrimidin-2- yl)amino)piperidin-1- yl)sulfonyl)-benzyl)-2,6- diazaspiro[3.3]heptan-2- yl)-1-oxoisoindolin-2- yl)piperidine-2,6-dione | 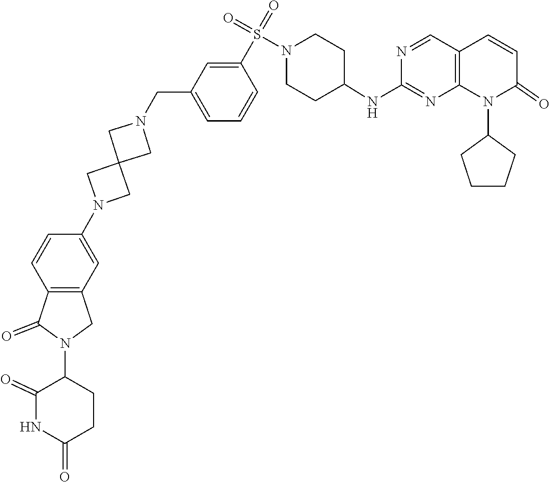 [SMILES: O=C1CCC(N2Cc3cc(N4CC5(CN(Cc6cccc(S(=O)(=O)N7CCC(Nc8ncc9ccc(=O)n(C%10CCCC%10)c9n8)CC7)c6)C5)C4)ccc3C2=O)C(=O)N1] | [M + 1]⁺ = 806.4 |
| Example 61 | 3-(5-(6-(3-((4-((8- (bicyclo[3.1.0]hexan-3- yl)-7-oxo-7,8-dihydro- pyrido[2,3-d]pyrimidin- 2-yl)amino)piperidin-1- yl)sulfonyl)-benzyl)-2,6- diazaspiro[3.3]heptan-2- yl)-1-oxoisoindolin-2- yl)piperidine-2,6-dione | 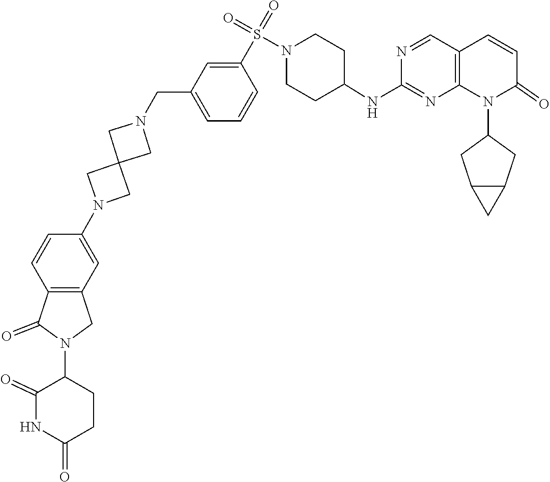 [SMILES: O=C1CCC(N2Cc3cc(N4CC5(CN(Cc6cccc(S(=O)(=O)N7CCC(Nc8ncc9ccc(=O)n(C%10CC%11CC%11C%10)c9n8)CC7)c6)C5)C4)ccc3C2=O)C(=O)N1] | [M + 1]⁺ = 818.4 |
| Example 62 | 3-(5-(6-(3-((4-((8- (bicyclo[1.1.1]pentan-1- yl)-7-oxo-7,8- dihydropyrido-[2,3- d]pyrimidin-2- yl)amino)-piperidin-1- yl)sulfonyl)benzyl)-2,6- diazaspiro-[3.3]heptan- 2-yl)-1-oxoisoindolin-2- yl)piperidine-2,6-dione | 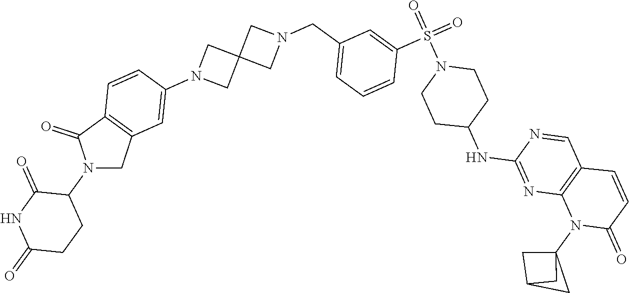 [SMILES: O=C1CCC(N2Cc3cc(N4CC5(CN(Cc6cccc(S(=O)(=O)N7CCC(Nc8ncc9ccc(=O)n(C%10%11CC(C%10)C%11)c9n8)CC7)c6)C5)C4)ccc3C2=O)C(=O)N1] | [M + 1]⁺ = 804.3 |

### Example 63

### Synthesis of 5-(6-(3-(((3R,4S)-4-((8-cyclopentyl-7-oxo-7,8-dihydropyrido[2,3-d]pyrimidin-2-yl)-amino)-3-fluoropiperidin-1-yl)sulfonyl)benzyl)-2,6-diazaspiro[3.3]heptan-2-yl)-2-(2,6-dioxopiperidin-3-yl)-6-fluoroisoindoline-1,3-dione

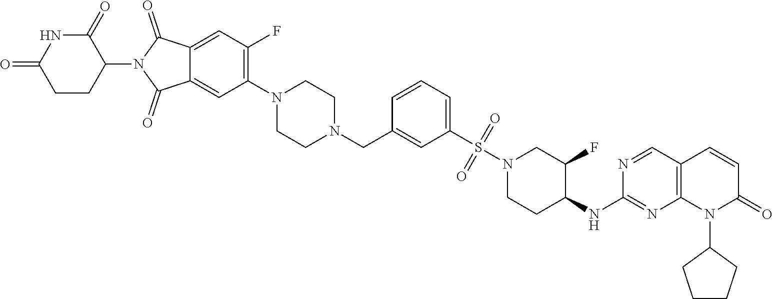 [SMILES: O=C1CCC(N2C(=O)c3cc(F)c(N4CCN(Cc5cccc(S(=O)(=O)N6CC[C@H](Nc7ncc8ccc(=O)n(C9CCCC9)c8n7)[C@H](F)C6)c5)CC4)cc3C2=O)C(=O)N1]

### Step 1: tert-butyl ((3R,4S)-1-((3-(bromomethyl)phenyl)sulfonyl)-3-fluoropiperidin-4-yl)carbamate

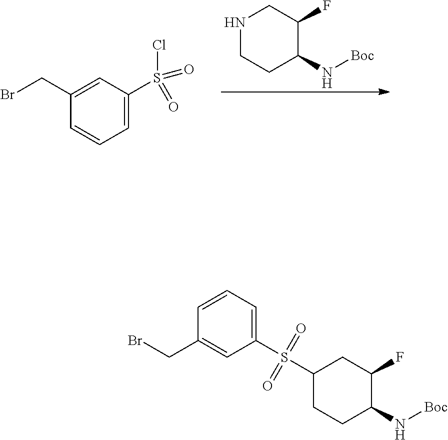 [SMILES: O=S(=O)(Cl)c1cccc(CBr)c1 ; CC(C)(C)OC(=O)N[C@H]1CCNC[C@H]1F ; CC(C)(C)OC(=O)N[C@H]1CCC(S(=O)(=O)c2cccc(CBr)c2)C[C@H]1F]

To a solution of tert-butyl ((3R,4S)-3-fluoropiperidin-4-yl)carbamate (100 mg, 0.46 mmol, 1.00 eq.) and TEA (93 mg, 0.92 mmol, 2.00 eq.) in THF (2.0 mL) was added 3-(bromomethyl)-benzene-1-sulfonyl chloride (122 mg, 0.46 mmol, 1.00 eq.) in THF (1.0 mL) slowly at −10° C. The mixture was stirred at −10° C. for 3 h and then extracted with EA and water. The organic layer was washed with brine, dried over Na₂SO₄, concentrated and the residue was purified by flash chromatography (PE:EA=4:1) to give the title compound (120 mg, 57.9%) as a white solid.

### Step 2: tert-butyl ((3R,4S)-1-((3-((4-(2-(2,6-dioxopiperidin-3-yl)-6-fluoro-1,3-dioxoisoindolin-5-yl)piperazin-1-yl)methyl)phenyl)sulfonyl)-3-fluoropiperidin-4-yl)carbamate

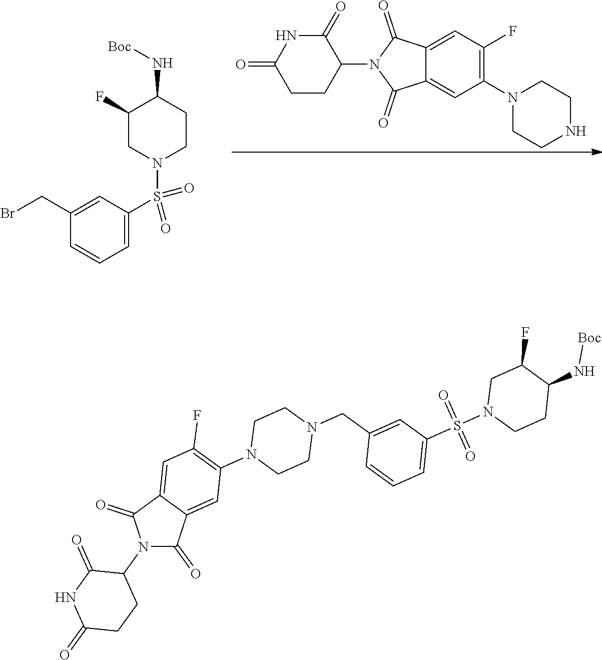 [SMILES: CC(C)(C)OC(=O)N[C@H]1CCN(S(=O)(=O)c2cccc(CBr)c2)C[C@H]1F ; O=C1CCC(N2C(=O)c3cc(F)c(N4CCNCC4)cc3C2=O)C(=O)N1 ; CC(C)(C)OC(=O)N[C@H]1CCN(S(=O)(=O)c2cccc(CN3CCN(c4cc5c(cc4F)C(=O)N(C4CCC(=O)NC4=O)C5=O)CC3)c2)C[C@H]1F]

To a solution of 2-(2,6-dioxopiperidin-3-yl)-5-fluoro-6-(piperazin-1-yl)isoindoline-1,3-dione (94 mg, 0.26 mmol, 1.00 eq.) and tert-butyl ((3R,4S)-1-((3-(bromomethyl)phenyl)-sulfonyl)-3-fluoropiperidin-4-yl)carbamate (141 mg, 0.31 mmol, 1.20 eq.) in THF (4.0 mL) was added TEA (131 mg, 1.30 mmol, 5.00 eq.) and the mixture was stirred at 55° C. overnight. The mixture was extracted DCM and the organic layer was washed with brine, dried over Na₂SO₄, and concentrated. The residue was purified by flash chromatography (DCM:MeOH=20:1) to give the title compound (120 mg, 63.1%) as a yellow solid.

### Step 3: 5-(6-(3-(((3R,4S)-4-((8-cyclopentyl-7-oxo-7,8-dihydropyrido[2,3-d]pyrimidin-2-yl)-amino)-3-fluoropiperidin-1-yl)sulfonyl)benzyl)-2,6-diazaspiro[3.3]heptan-2-yl)-2-(2,6-dioxopiperidin-3-yl)-6-fluoroisoindoline-1,3-dione

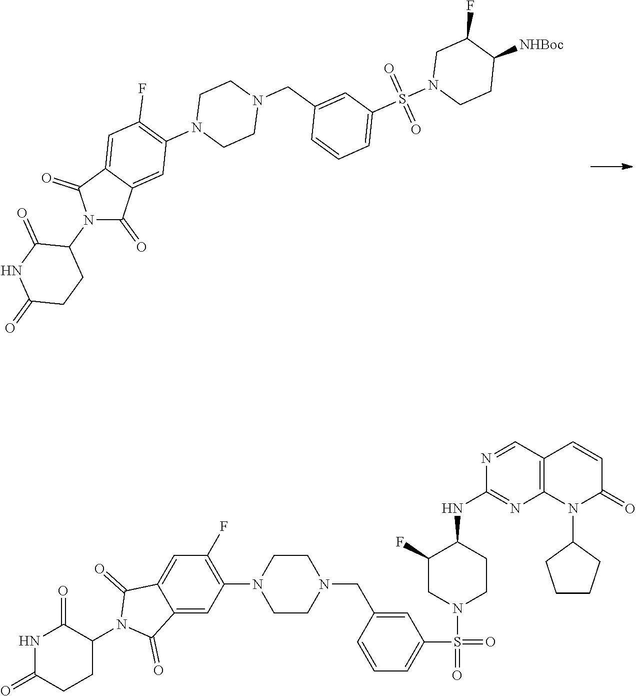 [SMILES: CC(C)(C)OC(=O)N[C@H]1CCN(S(=O)(=O)c2cccc(CN3CCN(c4cc5c(cc4F)C(=O)N(C4CCC(=O)NC4=O)C5=O)CC3)c2)C[C@H]1F ; O=C1CCC(N2C(=O)c3cc(F)c(N4CCN(Cc5cccc(S(=O)(=O)N6CC[C@H](Nc7ncc8ccc(=O)n(C9CCCC9)c8n7)[C@H](F)C6)c5)CC4)cc3C2=O)C(=O)N1]

tert-butyl ((3R,4S)-1-((3-((4-(2-(2,6-Dioxopiperidin-3-yl)-6-fluoro-1,3-dioxoisoindolin-5-yl)piperazin-1-yl)methyl)phenyl)sulfonyl)-3-fluoropiperidin-4-yl)carbamate was converted to the title compound by proceeding analogously as described in Example 7, Steps 5-6. MS (ES, m/z): [M+1]⁺=844.4

Compounds of Example 64-65 were prepared by proceeding analogously as described in Example 63.

| | | | |
|---|---|---|---|
| Example 64 | 5-(4-(3-(((3S,4S)- 4-((8-cyclopentyl- 7-oxo-7,8- dihydropyrido[2,3-d] pyrimidin-2-yl) amino)-3- methoxypiperidin- 1-yl)sulfonyl)- benzyl)piperazin- 1-yl)-2-(2,6- dioxopiperidin-3- yl)-6-fluoro- isoindoline-1,3- dione | 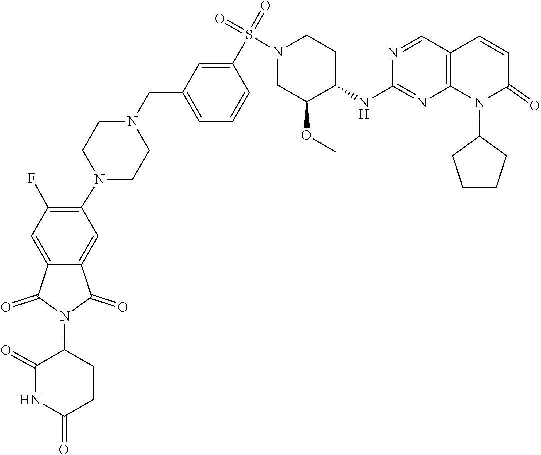 [SMILES: CO[C@H]1CN(S(=O)(=O)c2cccc(CN3CCN(c4cc5c(cc4F)C(=O)N(C4CCC(=O)NC4=O)C5=O)CC3)c2)CC[C@@H]1Nc1ncc2ccc(=O)n(C3CCCC3)c2n1] | [M + 1]⁺ = 856.4 |
| Example 65 | 5-(4-(3-(((3S,4S)- 4-((8-cyclopentyl- 7-oxo-7,8- dihydropyrido[2,3-d] pyrimidin-2- yl)amino)-3- fluoropiperidin-1- yl)sulfonyl)- benzyl)piperazin- 1-yl)-2-(2,6- dioxopiperidin-3- yl)-6-fluoro- isoindoline-1,3- dione | 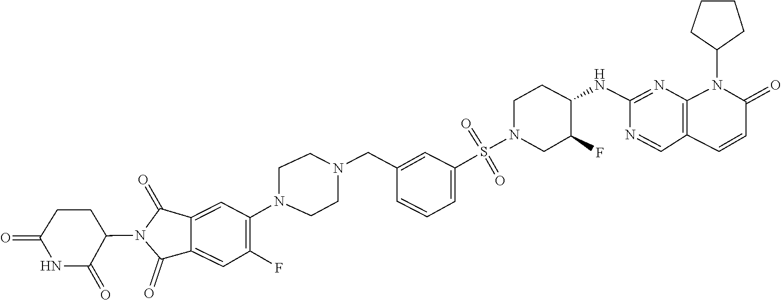 [SMILES: O=C1CCC(N2C(=O)c3cc(F)c(N4CCN(Cc5cccc(S(=O)(=O)N6CC[C@H](Nc7ncc8ccc(=O)n(C9CCCC9)c8n7)[C@@H](F)C6)c5)CC4)cc3C2=O)C(=O)N1] | [M + 1]⁺ = 844.3 |

### Example 66

### Synthesis of 3-(5-((4-(3-((4-((8-cyclopentyl-7-oxo-7,8-dihydropyrido[2,3-d]pyrimidin-2-yl)-amino)piperidin-1-yl)sulfonyl)phenoxy)piperidin-1-yl)methyl)-1-oxoisoindolin-2-yl)piperidine-2,6-dione

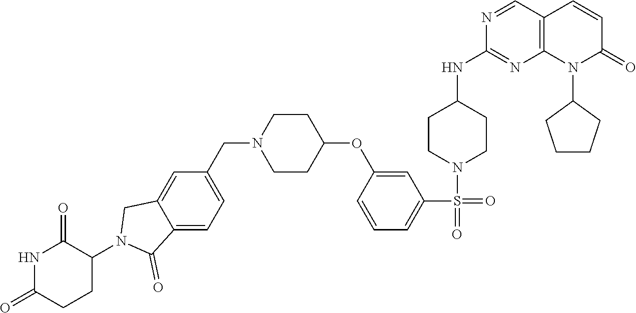 [SMILES: O=C1CCC(N2Cc3cc(CN4CCC(Oc5cccc(S(=O)(=O)N6CCC(Nc7ncc8ccc(=O)n(C9CCCC9)c8n7)CC6)c5)CC4)ccc3C2=O)C(=O)N1]

### Step 1: 2-(2,6-dioxopiperidin-3-yl)-1-oxoisoindoline-5-carbonitrile

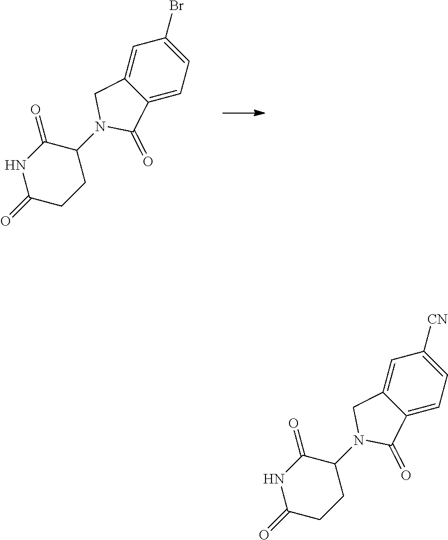 [SMILES: O=C1CCC(N2Cc3cc(Br)ccc3C2=O)C(=O)N1 ; N#Cc1ccc2c(c1)CN(C1CCC(=O)NC1=O)C2=O]

To a solution of 3-(5-bromo-1-oxoisoindolin-2-yl)piperidine-2,6-dione (2.00 g, 6.21 mmol, 1.00 eq.) and Zn(CN)₂ (438 mg, 3.73 mmol, 0.60 eq.) in DMF (30.0 mL) was added Pd(pph₃)₄ (714 mg). The mixture was stirred at 100° C. overnight. The mixture was extracted with DCM and purified by silica gel column chromatography eluting with PE/EtOAc (1:2) to give the title compound (1.20 g, 71.9%) as yellow solid.

### Step 2: 2-(2,6-dioxopiperidin-3-yl)-1-oxoisoindoline-5-carbaldehyde

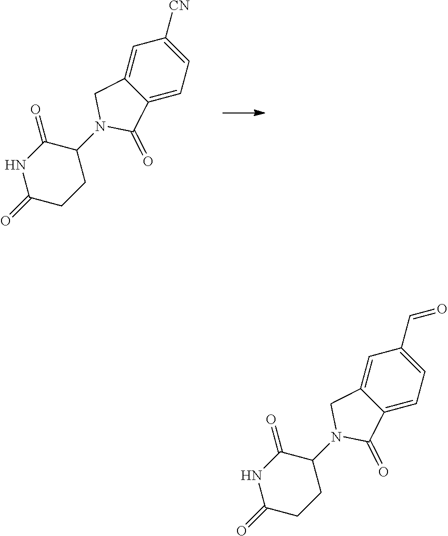 [SMILES: N#Cc1ccc2c(c1)CN(C1CCC(=O)NC1=O)C2=O ; O=Cc1ccc2c(c1)CN(C1CCC(=O)NC1=O)C2=O]

A mixture of 2-(2,6-dioxopiperidin-3-yl)-1-oxoisoindoline-5-carbonitrile (1.20 g, 4.46 mmol, 1.00 eq.), NaH₂PO₂·H₂O (993 mg, 9.37 mmol, 2.10 eq.) and Raney-Ni (500 mg) in pyridine: H₂O:AcOH (40.0 mL, 2:2:1) was stirred at 70° C. overnight. The reaction mixture was filtered and washed with aq. NaHCO₃. The solution was extracted with DCM and the organic layer was concentrated. The residue was purified by silica gel column chromatography eluting PE/EtOAc (1:2) to give the title compound (260 mg, 21.5%) as yellow solid.

### Step 3: benzyl 4-(3-((4-((tert-butoxycarbonyl)amino)piperidin-1-yl)sulfonyl)phenoxy)piperidine-1-carboxylate

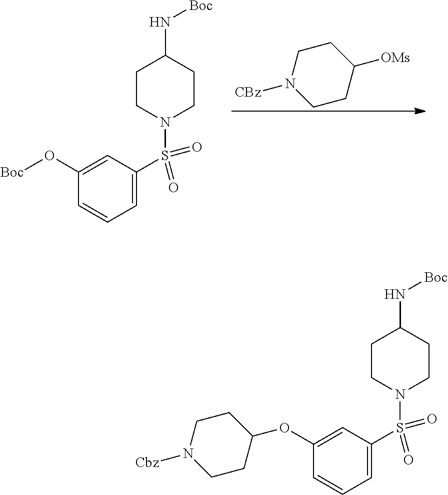

To a solution of tert-butyl (1-((3-((tert-butoxycarbonyl)oxy)phenyl)sulfonyl)piperidin-4-yl)carbamate (7.30 g, 16.0 mmol, 1.00 eq.) in DMSO (70.0 mL) were added benzyl 4-((methylsulfonyl)oxy)piperidine-1-carboxylate (7.52 g, 24 mmol, 1.50 eq.) and Cs₂CO₃ (10.4 g, 32 mmol, 2.00 eq.). The reaction mixture was stirred at 90° C. for 4 h and then extracted with EtOAc. The organic layer was concentrated and. the crude product was purified by silica gel column chromatography eluting PE/EtOAc (3:1) to give the title compound (6.0 g, 65.4%) as a yellow solid.

### Step 4: tert-butyl (1-((3-(piperidin-4-yloxy)phenyl)sulfonyl)piperidin-4-yl)carbamate

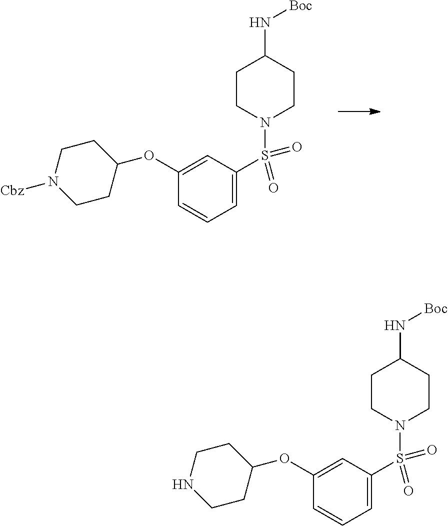 [SMILES: CC(C)(C)OC(=O)NC1CCN(S(=O)(=O)c2cccc(OC3CCN(C(=O)OCc4ccccc4)CC3)c2)CC1 ; CC(C)(C)OC(=O)NC1CCN(S(=O)(=O)c2cccc(OC3CCNCC3)c2)CC1]

A mixture of benzyl 4-(3-((4-((tert-butoxycarbonyl)amino)piperidin-1-yl)sulfonyl)-phenoxy)piperidine-1-carboxylate (6.0 g, 10.47 mmol, 1.00 eq.), HCOONH₄ (3.3 g, 52.35 mmol, 5.00 eq.), and Pd(OH)₂ (1.2 g) in EtOH (60.0 mL) was stirred at 70° C. for 4 h. The mixture was filtered and concentrated to give the title compound (4.6 g, crude) as a white solid.

### Step 5: tert-butyl (1-((3-((1-((2-(2,6-dioxopiperidin-3-yl)-1-oxoisoindolin-5-yl)methyl)piperidin-4-yl)oxy)phenyl)sulfonyl)piperidin-4-yl)carbamate

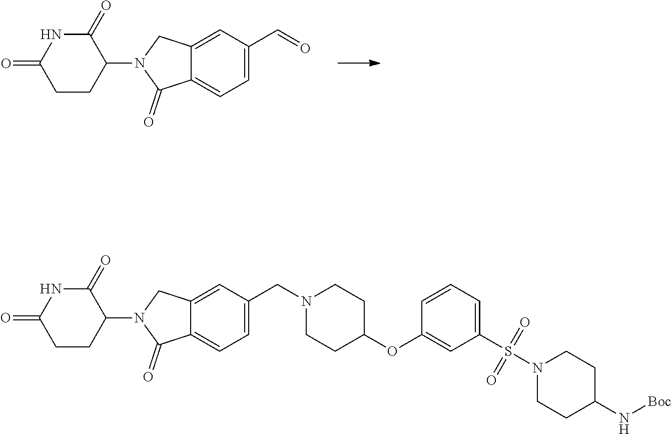 [SMILES: O=Cc1ccc2c(c1)CN(C1CCC(=O)NC1=O)C2=O ; CC(C)(C)OC(=O)NC1CCN(S(=O)(=O)c2cccc(OC3CCN(Cc4ccc5c(c4)CN(C4CCC(=O)NC4=O)C5=O)CC3)c2)CC1]

To a mixture of 2-(2,6-dioxopiperidin-3-yl)-1-oxoisoindoline-5-carbaldehyde (100 mg, 0.37 mmol, 1.00 eq.) in THF (3.0 mL) were added tert-butyl (1-((3-(piperidin-4-yloxy)phenyl)-sulfonyl)piperidin-4-yl)carbamate (169 mg, 0.39 mmol, 1.05 eq.) and 1 drop of AcOH. The mixture was stirred at 40° C. for 0.5 h. NaBH₃CN (47 mg, 0.74 mmol, 2.00 eq.) was added and stirring was continued at RT for 16 h. The reaction mixture was extracted with DCM and the organic layer was separated and concentrated. The residue was purified by silica gel column chromatography eluting with DCM/MeOH (20:1) to give the title compound (120 mg, 46.7%) as a yellow solid.

### Step 6: 3-(5-((4-(3-((4-((8-cyclopentyl-7-oxo-7,8-dihydropyrido[2,3-d]pyrimidin-2-yl)amino)piperidin-1-yl)sulfonyl)phenoxy)piperidin-1-yl)methyl)-1-oxoisoindolin-2-yl)piperidine-2,6-dione

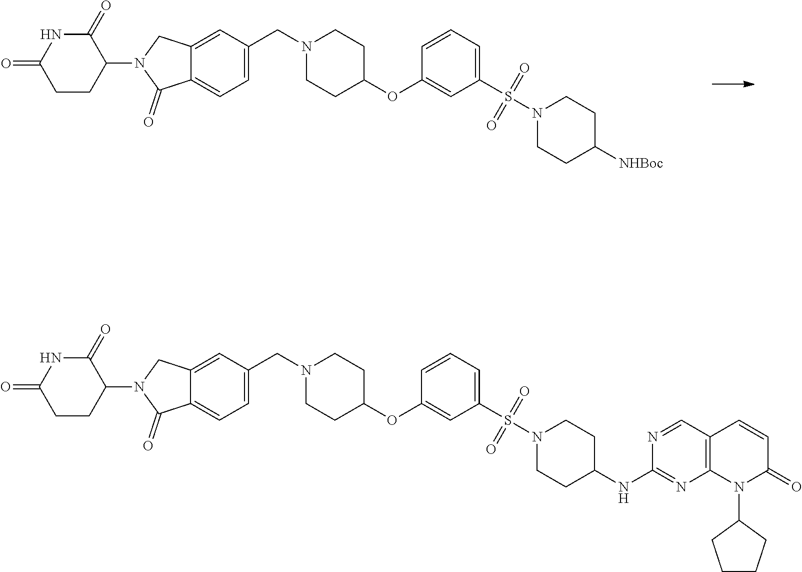 [SMILES: CC(C)(C)OC(=O)NC1CCN(S(=O)(=O)c2cccc(OC3CCN(Cc4ccc5c(c4)CN(C4CCC(=O)NC4=O)C5=O)CC3)c2)CC1 ; O=C1CCC(N2Cc3cc(CN4CCC(Oc5cccc(S(=O)(=O)N6CCC(Nc7ncc8ccc(=O)n(C9CCCC9)c8n7)CC6)c5)CC4)ccc3C2=O)C(=O)N1]

tert-butyl (1-((3-((1-((2-(2,6-Dioxopiperidin-3-yl)-1-oxoisoindolin-5-yl)methyl)piperidin-4-yl)oxy)phenyl)sulfonyl)piperidin-4-yl)carbamate was converted to the title compound by proceeding analogously as described in Example 7, Steps 5-6. MS (ES, m/z): [M+1]⁺=809.5

Compounds of Example 67-68 were prepared by proceeding analogously as described in Example 66.

| | | | |
|---|---|---|---|
| Example 67 | 3-(5-((3-(3-((4-((8- cyclopentyl-7-oxo- 7,8- dihydropyrido[2,3- d]pyrimidin-2- yl)amino)piperidin- 1-yl)-sulfonyl)- phenoxy)azetidin-1- yl)methyl)-1- oxoisoindolin-2- yl)piperidine-2,6- dione | 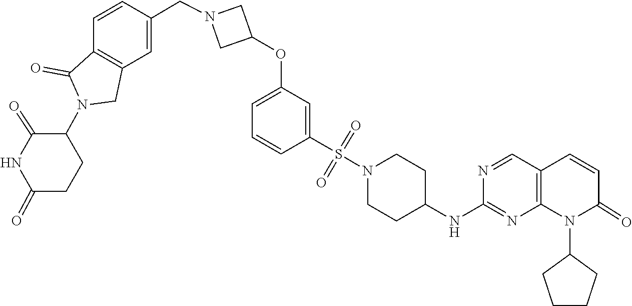 [SMILES: O=C1CCC(N2Cc3cc(CN4CC(Oc5cccc(S(=O)(=O)N6CCC(Nc7ncc8ccc(=O)n(C9CCCC9)c8n7)CC6)c5)C4)ccc3C2=O)C(=O)N1] | [M + 1]⁺ = 781.3 |
| Example 68 | 3-(5-(((2-(3-((4-((8- cyclopentyl-7-oxo- 7,8- dihydropyrido[2,3- d]pyrimidin-2- yl)amino)piperidin- 1-yl)- sulfonyl)phenoxy) ethyl)amino)methyl)- 1-oxoisoindolin-2- yl)piperidine-2,6- dione | 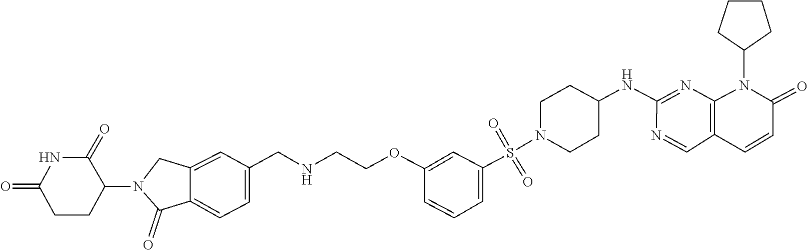 [SMILES: O=C1CCC(N2Cc3cc(CNCCOc4cccc(S(=O)(=O)N5CCC(Nc6ncc7ccc(=O)n(C8CCCC8)c7n6)CC5)c4)ccc3C2=O)C(=O)N1] | [M + 1]⁺ = 769.4 |

### Example 69

### Synthesis of 3-(5-(3-(4-(3-((4-((8-cyclopentyl-7-oxo-7,8-dihydropyrido[2,3-d]pyrimidin-2-yl)-amino)piperidin-1-yl)sulfonyl)phenyl)piperazin-1-yl)azetidin-1-yl)-1-oxoisoindolin-2-yl)-piperidine-2,6-dione

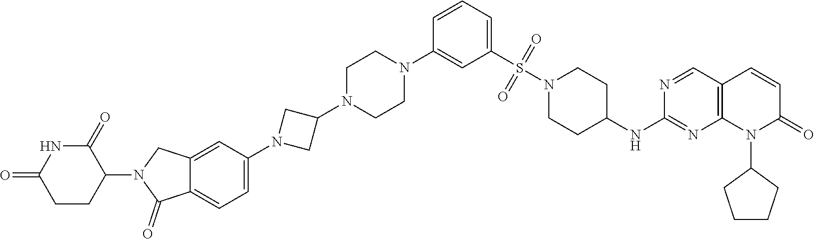 [SMILES: O=C1CCC(N2Cc3cc(N4CC(N5CCN(c6cccc(S(=O)(=O)N7CCC(Nc8ncc9ccc(=O)n(C%10CCCC%10)c9n8)CC7)c6)CC5)C4)ccc3C2=O)C(=O)N1]

### Step 1: benzyl 3-(4-(3-((4-((tert-butoxycarbonyl)amino)piperidin-1-yl)sulfonyl)phenyl)piperazin-1-yl)azetidine-1-carboxylate

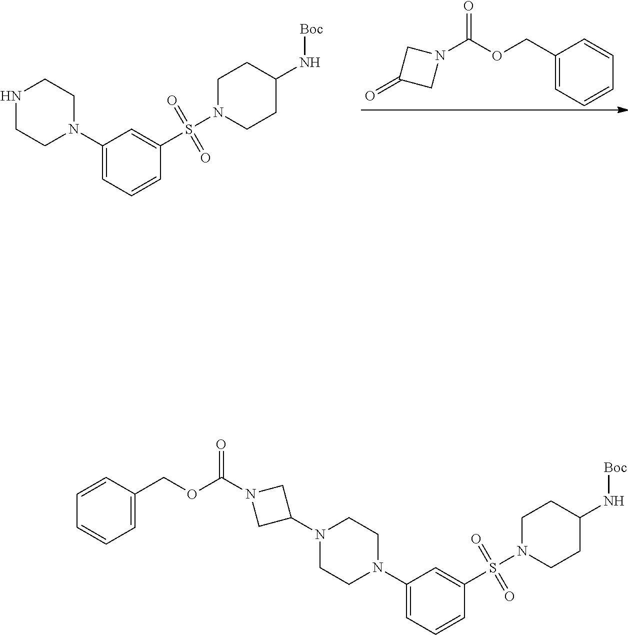 [SMILES: CC(C)(C)OC(=O)NC1CCN(S(=O)(=O)c2cccc(N3CCNCC3)c2)CC1 ; O=C1CN(C(=O)OCc2ccccc2)C1 ; CC(C)(C)OC(=O)NC1CCN(S(=O)(=O)c2cccc(N3CCN(C4CN(C(=O)OCc5ccccc5)C4)CC3)c2)CC1]

To a mixture of tert-butyl (1-((3-(piperazin-1-yl)phenyl)sulfonyl)piperidin-4-yl)carbamate (740 mg, 1.75 mmol, 1.00 eq.) in THF (10.0 mL) were added AcOH (3 drops) and benzyl 3-oxoazetidine-1-carboxylate (718 mg, 3.50 mmol, 2.00 eq.). The solution was stirred at 45° C. for 0.5 h. The solution was cooled to RT and NaBH₃CN (220 mg, 3.50 mmol, 2.00 eq.) was added. The solution was stirred at RT overnight and then extracted with EtOAc. The organic layer was concentrated and the crude product was purified by silica gel column chromatography eluting DCM/MeOH (20:1) to give the title compound (375 mg, 35.0%) as a white oil.

### Step 2: tert-butyl (1-((3-(4-(1-(2-(2,6-dioxopiperidin-3-yl)-1-oxoisoindolin-5-yl)azetidin-3-yl)piperazin-1-yl)phenyl)sulfonyl)piperidin-4-yl)carbamate

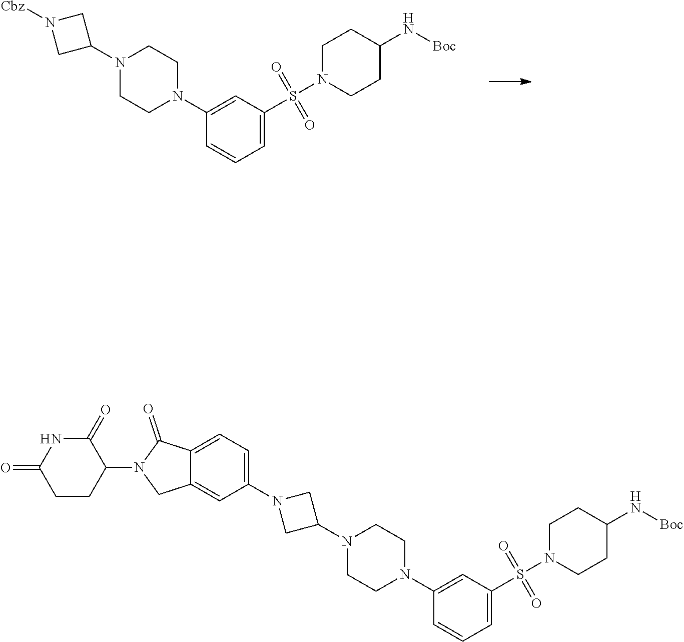 [SMILES: CC(C)(C)OC(=O)NC1CCN(S(=O)(=O)c2cccc(N3CCN(C4CN(C(=O)OCc5ccccc5)C4)CC3)c2)CC1 ; CC(C)(C)OC(=O)NC1CCN(S(=O)(=O)c2cccc(N3CCN(C4CN(c5ccc6c(c5)CN(C5CCC(=O)NC5=O)C6=O)C4)CC3)c2)CC1]

Benzyl 3-(4-(3-((4-((tert-butoxycarbonyl)amino)piperidin-1-yl)sulfonyl)phenyl)-piperazin-1-yl)azetidine-1-carboxylate was converted to the title compound using similar procedure as described in Example 34, Step 4-7.

### Step 3: 3-(5-(3-(4-(3-((4-((8-cyclopentyl-7-oxo-7,8-dihydropyrido[2,3-d]pyrimidin-2-yl)-amino)piperidin-1-yl)sulfonyl)phenyl)piperazin-1-yl)azetidin-1-yl)-1-oxoisoindolin-2-yl)piperidine-2,6-dione

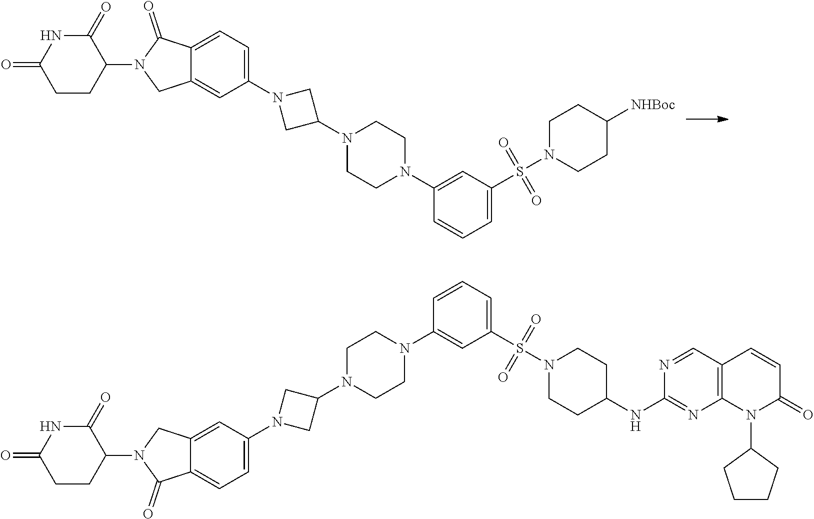

tert-butyl (1-((3-(4-(1-(2-(2,6-Dioxopiperidin-3-yl)-1-oxoisoindolin-5-yl)azetidin-3-yl)piperazin-1-yl)phenyl)sulfonyl)piperidin-4-yl)carbamate was converted to the title compound by proceeding analogously as described in Example 7, Steps 5-6. MS (ES, m/z): [M+1]⁺=835.5

### Example 70

### Synthesis of 3-(5-(4-(1-(3-((4-((8-cyclopentyl-7-oxo-7,8-dihydropyrido[2,3-d]pyrimidin-2-yl)-amino)piperidin-1-yl)sulfonyl)phenyl)azetidin-3-yl)piperazin-1-yl)-1-oxoisoindolin-2-yl)piperidine-2,6-dione

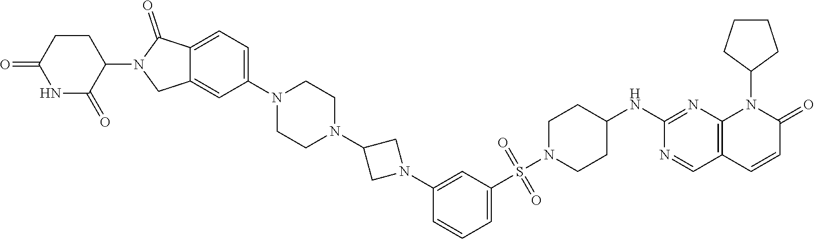 [SMILES: O=C1CCC(N2Cc3cc(N4CCN(C5CN(c6cccc(S(=O)(=O)N7CCC(Nc8ncc9ccc(=O)n(C%10CCCC%10)c9n8)CC7)c6)C5)CC4)ccc3C2=O)C(=O)N1]

### Step 1: 3-hydroxyazetidine

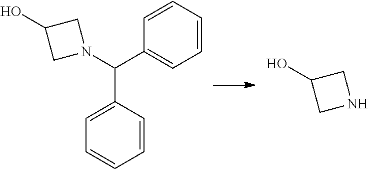 [SMILES: OC1CN(C(c2ccccc2)c2ccccc2)C1 ; OC1CNC1]

To a stirred solution of 1-benzhydrylazetidin-3-ol (5.00 g, 20.92 mmol, 1.00 eq.) and Pd(OH)₂, (3.50 g) in MeOH (130.00 mL) was added AcOH (18.50 mL). The resulting mixture was stirred at 50° C. under H₂ (50 psi) for 12 h. HCl (aq) was added to adjust the pH of the solution to pH 3. The solution was concentrated to give crude product (2.28 g, crude) as white oil.

### Step 2: tert-butyl (1-((3-(3-hydroxyazetidin-1-yl)phenyl)sulfonyl)piperidin-4-yl)carbamate

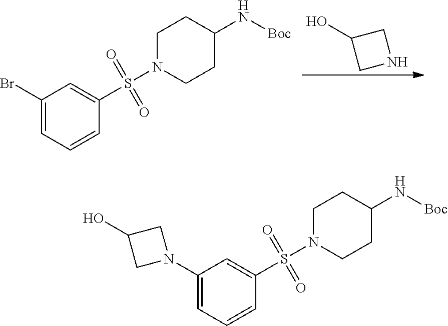 [SMILES: CC(C)(C)OC(=O)NC1CCN(S(=O)(=O)c2cccc(Br)c2)CC1 ; OC1CNC1 ; CC(C)(C)OC(=O)NC1CCN(S(=O)(=O)c2cccc(N3CC(O)C3)c2)CC1]

A mixture of tert-butyl (1-((3-bromophenyl)sulfonyl)piperidin-4-yl)carbamate (5.83 g, 13.95 mmol, 1.00 eq.), K₂CO₃ (6.74 g, 48.83 mmol, 3.50 eq.), CuI (0.53 g, 2.79 mmol, 0.20 eq.), L-PRO (481 mg, 4.19 mmol, 0.30 eq.) and 3-hydroxyazetidine (2.28 g, 20.92 mmol, 1.50 eq.) in DMSO (50.00 mL) was stirred at 90° C. for 12 h. The mixture was quenched with H₂O and extracted with EtOAc. The organic layer was concentrated and purified by silica gel column chromatography eluting with PE/EtOAc (2:1) to give the title compound (3.98 g, 69.5%) as white solid.

### Step 3: tert-butyl (1-((3-(3-oxoazetidin-1-yl)phenyl)sulfonyl)piperidin-4-yl)carbamate

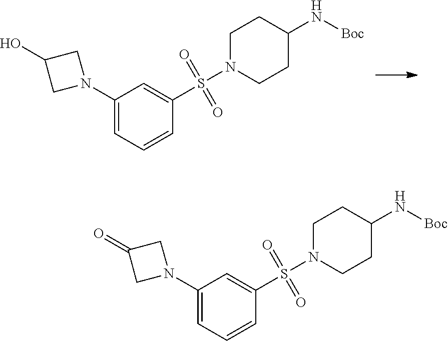 [SMILES: CC(C)(C)OC(=O)NC1CCN(S(=O)(=O)c2cccc(N3CC(O)C3)c2)CC1 ; CC(C)(C)OC(=O)NC1CCN(S(=O)(=O)c2cccc(N3CC(=O)C3)c2)CC1]

To a stirred solution of tert-butyl (1-((3-(3-hydroxyazetidin-1-yl)phenyl)sulfonyl)-piperidin-4-yl)carbamate (0.50 g, 1.22 mmol, 1.00 eq.) in DCM (5.00 mL) was added Dess-Martin (1.03 g, 2.44 mmol, 2.00 eq.) and the mixture was stirred at 0° C. for 3 h. The mixture was diluted with sodium thiosulfate (aq) and extracted with DCM. The organic layer was concentrated and the residue was purified by silica gel column chromatography, eluted with EtOAc/PE (3:1), to give the title compound (50.00 mg, 10.0%) as a white solid.

### Step 4: tert-butyl (1-((3-(3-(4-(2-(2,6-dioxopiperidin-3-yl)-1-oxoisoindolin-5-yl)piperazin-1-yl)-azetidin-1-yl)phenyl)sulfonyl)piperidin-4-yl)carbamate

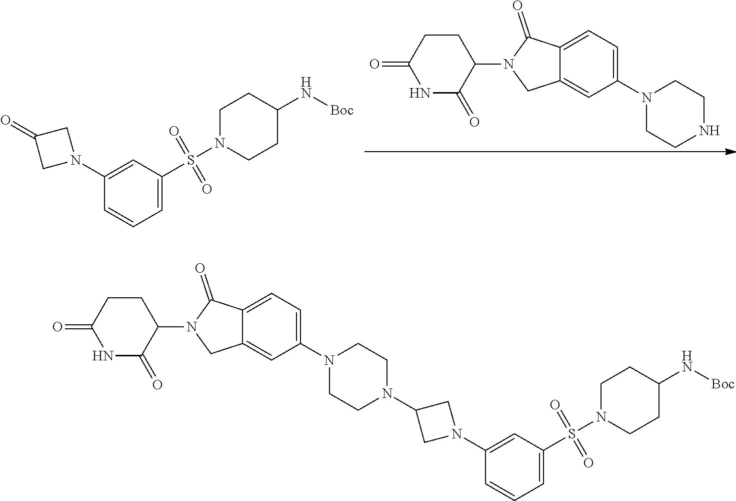 [SMILES: CC(C)(C)OC(=O)NC1CCN(S(=O)(=O)c2cccc(N3CC(=O)C3)c2)CC1 ; O=C1CCC(N2Cc3cc(N4CCNCC4)ccc3C2=O)C(=O)N1 ; CC(C)(C)OC(=O)NC1CCN(S(=O)(=O)c2cccc(N3CC(N4CCN(c5ccc6c(c5)CN(C5CCC(=O)NC5=O)C6=O)CC4)C3)c2)CC1]

To a solution of tert-butyl (1-((3-(3-oxoazetidin-1-yl)phenyl)sulfonyl)piperidin-4-yl)carbamate (40.00 mg, 0.10 mmol, 1.00 eq.) in THF (1.00 mL) and DMF (0.50 mL) were added AcOH (3 drops) and 3-(1-oxo-5-(piperazin-1-yl)isoindolin-2-yl)piperidine-2,6-dione (39.00 mg, 0.12 mmol, 1.20 eq.). The solution was stirred at 45° C. for 45 min. The solution cooled to RT and NaBH₃CN (13.00 mg, 0.20 mmol, 2.00 eq.) was added. The mixture was stirred at RT for 12 h and then diluted with water and extracted with EtOAc. The combined organic layer was washed with brine, dried over anhydrous Na₂SO₄ and concentrated. The residue was purified by TLC, eluted with DCM/MeOH (20:1), to afford the title compound (52.00 mg, 72.2%) as a white solid.

### Step 5: 3-(5-(4-(1-(3-((4-((8-cyclopentyl-7-oxo-7,8-dihydropyrido[2,3-d]pyrimidin-2-yl)-amino)piperidin-1-yl)sulfonyl)phenyl)azetidin-3-yl)piperazin-1-yl)-1-oxoisoindolin-2-yl)piperidine-2,6-dione

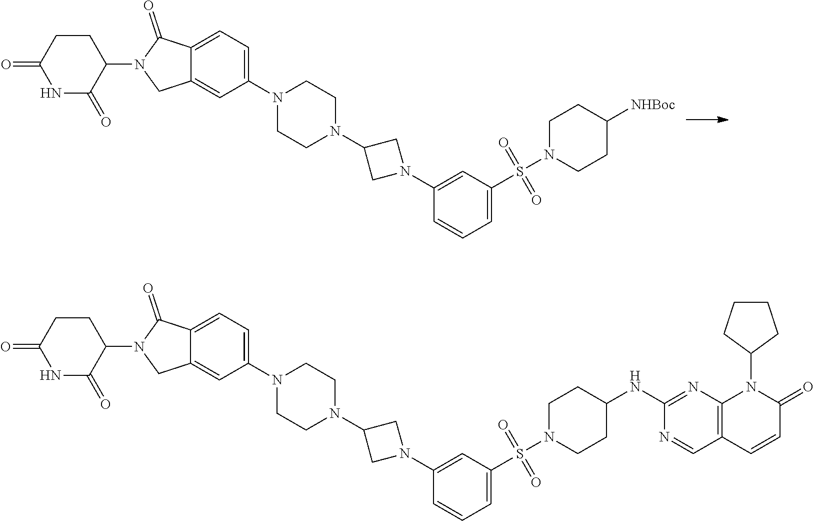 [SMILES: CC(C)(C)OC(=O)NC1CCN(S(=O)(=O)c2cccc(N3CC(N4CCN(c5ccc6c(c5)CN(C5CCC(=O)NC5=O)C6=O)CC4)C3)c2)CC1 ; O=C1CCC(N2Cc3cc(N4CCN(C5CN(c6cccc(S(=O)(=O)N7CCC(Nc8ncc9ccc(=O)n(C%10CCCC%10)c9n8)CC7)c6)C5)CC4)ccc3C2=O)C(=O)N1]

tert-butyl (1-((3-(3-(4-(2-(2,6-Dioxopiperidin-3-yl)-1-oxoisoindolin-5-yl)piperazin-1-yl)azetidin-1-yl)phenyl)sulfonyl)piperidin-4-yl)carbamate was converted to the title compound by proceeding analogously as described in Example 7, Steps 5-6. MS (ES, m/z): [M+1]⁺=835.

### Example 71

### Synthesis of 1-(6-(1-(3-((4-((8-cyclopentyl-7-oxo-7,8-dihydropyrido[2,3-d]pyrimidin-2-yl)-amino)piperidin-1-yl)sulfonyl)benzyl)piperidin-4-yl)-1-methyl-1H-indazol-3-yl)-dihydropyrimidine-2,4 (1H,3H)-dione

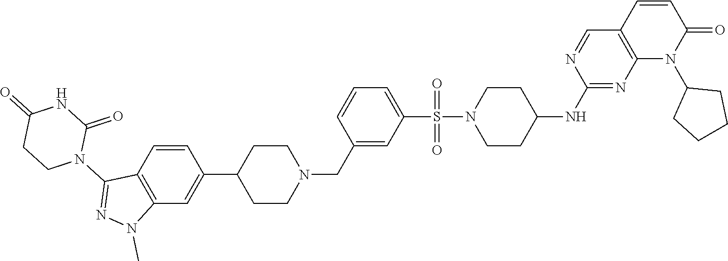 [SMILES: Cn1nc(N2CCC(=O)NC2=O)c2ccc(C3CCN(Cc4cccc(S(=O)(=O)N5CCC(Nc6ncc7ccc(=O)n(C8CCCC8)c7n6)CC5)c4)CC3)cc21]

### Step 1: 6-bromo-1-methyl-1H-indazol-3-amine

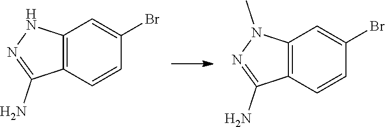

To a stirred solution of 6-bromo-1H-indazol-3-amine (5.60 g, 26.42 mmol, 1.00 eq.) in DMF (20.0 mL) was added NaH (2.10 g, 52.83 mmol, 2.00 eq.) at 0° C. and stirring was continued at 0° C. for 1 h. CH₃I (4.10 g, 29.06 mmol, 1.10 eq.) was added and the resulting mixture was stirred at RT for 3 h under N₂. The mixture was poured into cold water, filtered and washed with water, dried to give the title compound (5.40 g, 90.5%) as yellow solid.

### Step 2: 3-((6-bromo-1-methyl-1H-indazol-3-yl)amino)propanoic acid

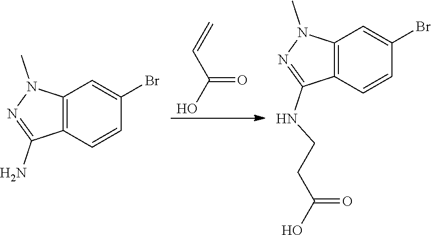

To a stirred solution of 6-bromo-1-methyl-1H-indazol-3-amine (5.00 g, 22.12 mmol, 1.00 eq.) in AcOH (3.17 g, 52.43 mmol, 2.37 eq.) and water (5.0 mL) was added acrylic acid (1.60 g, 22.12 mmol, 1.00 eq.). The resulting mixture was stirred at 105° C. for 20 h under N2. The mixture was poured into cold water, the pH was adjusted to 6-7 with 6N HCl. The product was extracted with EtOAc and the organic layer was washed with water, dried to give the title compound (3.11 g, 47.2%) as yellow solid.

### Step 3: 1-(6-bromo-1-methyl-1H-indazol-3-yl)dihydropyrimidine-2,4 (1H,3H)-dione

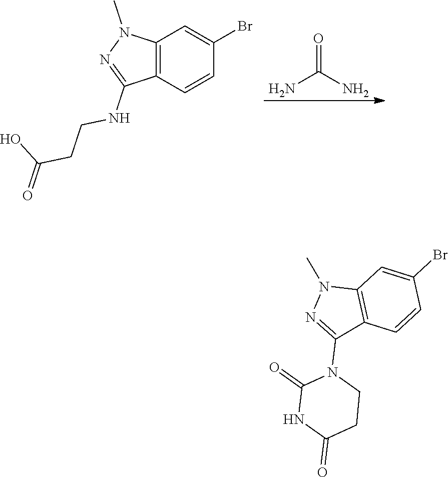

A solution of 3-((6-bromo-1-methyl-1H-indazol-3-yl)amino)propanoic acid (3.11 g, 1.90 mmol, 1.00 eq.) and urea (3.02 g, 50.31 mmol, 5.00 eq.) in AcOH (30.0 mL) was stirred at 120° C. for 20 h under N₂. After cooling the mixture to room temperature, con. HCl (6.0 mL) was added and the reaction was heated again for 30 min. The crude mixture was purified by flash column chromatography (EA:PE=0 to 100%) to give the title compound (0.81 g, 25.0%) as yellow solid.

### Step 4: tert-butyl 4-(3-(2,4-dioxotetrahydropyrimidin-1 (2H)-yl)-1-methyl-1H-indazol-6-yl)-5,6-dihydropyridine-1 (2H)-carboxylate

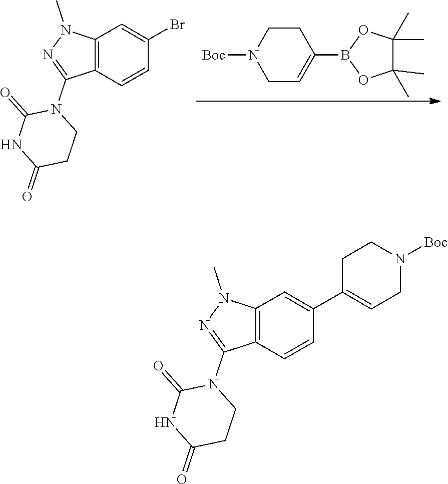 [SMILES: Cn1nc(N2CCC(=O)NC2=O)c2ccc(Br)cc21 ; CC(C)(C)OC(=O)N1CC=C(B2OC(C)(C)C(C)(C)O2)CC1 ; Cn1nc(N2CCC(=O)NC2=O)c2ccc(C3=CCN(C(=O)OC(C)(C)C)CC3)cc21]

To a mixture of 1-(6-bromo-1-methyl-1H-indazol-3-yl)dihydropyrimidine-2,4 (1H,3H)-dione (1.10 g, 3.41 mmol, 1.00 eq.) in 1,4-dioxane/H₂O (10 mL/1 mL) were added tert-butyl 4-(4,4,5,5-tetramethyl-1,3,2-dioxaborolan-2-yl)-5,6-dihydropyridine-1 (2H)-carboxylate (1.60 g, 5.11 mmol, 1.50 eq.), K₃PO₄ (2.20 g, 10.22 mmol, 3.00 eq.) and X-phos-G3 (289 mg, 0.34 mmol, 0.10 eq.). The mixture was stirred at 60° C. under N₂ for 3 h. The mixture was diluted with DCM, washed with water (and brine, dried over Na₂SO₄, and concentrated. The residue was purified by column chromatography on silica gel (DCM:MeOH=20:1) to give the title compound (1.00 g, 69.0%) as yellow solid.

### Step 5: tert-butyl 4-(3-(2,4-dioxotetrahydropyrimidin-1 (2H)-yl)-1-methyl-1H-indazol-6-yl)-piperidine-1-carboxylate

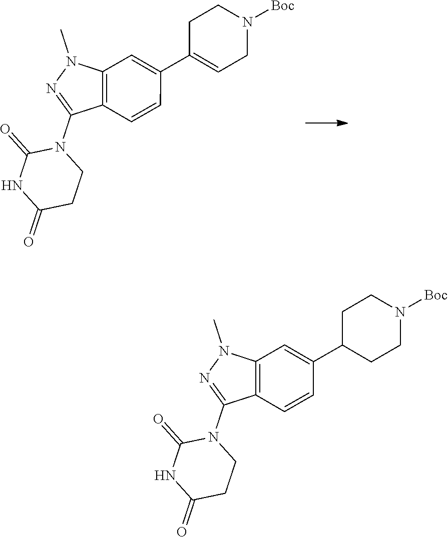 [SMILES: Cn1nc(N2CCC(=O)NC2=O)c2ccc(C3=CCN(C(=O)OC(C)(C)C)CC3)cc21 ; Cn1nc(N2CCC(=O)NC2=O)c2ccc(C3CCN(C(=O)OC(C)(C)C)CC3)cc21]

A mixture of tert-butyl 4-(3-(2,4-dioxotetrahydropyrimidin-1 (2H)-yl)-1-methyl-1H-indazol-6-yl)-5,6-dihydropyridine-1 (2H)-carboxylate (300 mg, 0.71 mmol, 1.00 eq.), Pd/C (150 mg, 50% wt) and Pd(OH)/C (150 mg, 50% wt) in THF (20 mL) was stirred under H₂ at 50° C. and 50 psi overnight. The mixture was filtered and the filtrate was concentrated and purified by column chromatography on silica gel (PE:EA=1:1) to give the title compound (120 mg, 39.9%) as yellow solid.

### Step 6: 1-(1-methyl-6-(piperidin-4-yl)-1H-indazol-3-yl)dihydropyrimidine-2,4 (1H,3H)-dione 2,2,2-trifluoroacetate

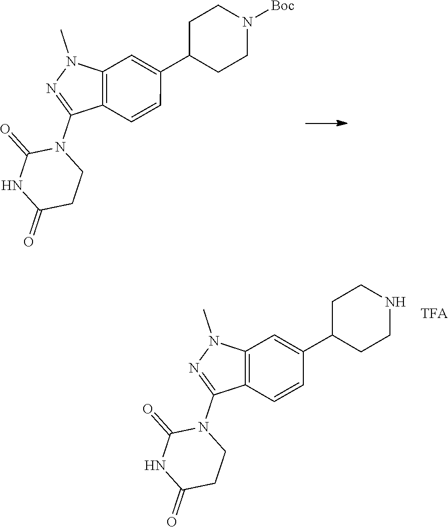 [SMILES: Cn1nc(N2CCC(=O)NC2=O)c2ccc(C3CCN(C(=O)OC(C)(C)C)CC3)cc21 ; Cn1nc(N2CCC(=O)NC2=O)c2ccc(C3CCNCC3)cc21 ; "TFA"]

A mixture of tert-butyl 4-(3-(2,4-dioxotetrahydropyrimidin-1 (2H)-yl)-1-methyl-1H-indazol-6-yl)piperidine-1-carboxylate (20 mg, 0.05 mmol, 1.00 eq.) in TFA/DCM (0.5 mL/2 mL) was stirred at RT for 2 h. The mixture was concentrated to give the title compound (20 mg, 96.6%) as brown oil.

### Step 7: 1-(6-(1-(3-((4-((8-cyclopentyl-7-oxo-7,8-dihydropyrido[2,3-d]pyrimidin-2-yl)amino)piperidin-1-yl)sulfonyl)benzyl)piperidin-4-yl)-1-methyl-1H-indazol-3-yl)dihydropyrimidine-2,4 (1H,3H)-dione

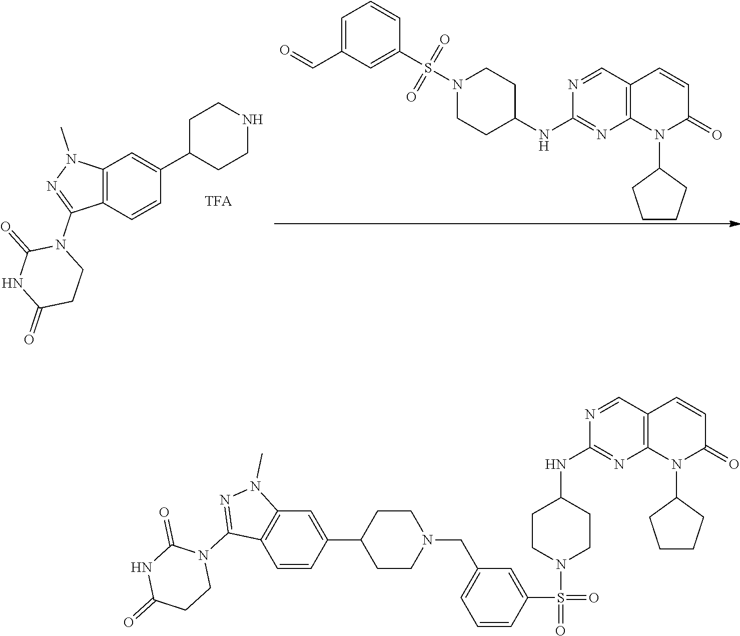 [SMILES: Cn1nc(N2CCC(=O)NC2=O)c2ccc(C3CCNCC3)cc21 ; O=Cc1cccc(S(=O)(=O)N2CCC(Nc3ncc4ccc(=O)n(C5CCCC5)c4n3)CC2)c1 ; Cn1nc(N2CCC(=O)NC2=O)c2ccc(C3CCN(Cc4cccc(S(=O)(=O)N5CCC(Nc6ncc7ccc(=O)n(C8CCCC8)c7n6)CC5)c4)CC3)cc21]

To a solution of 1-(1-methyl-6-(piperidin-4-yl)-1H-indazol-3-yl)dihydropyrimidine-2,4 (1H,3H)-dione 2,2,2-trifluoroacetate (20 mg, 0.04 mmol, 1.00 eq.) and TEA (3 mL) in THF/DMF (3 mL/1 mL) were added 3-((4-((8-cyclopentyl-7-oxo-7,8-dihydropyrido[2,3-d]pyrimidin-2-yl)amino)piperidin-1-yl)sulfonyl)benzaldehyde (20 mg, 0.04 mmol, 1.00 eq.) and AcOH (10 drops). The mixture was stirred at 40° C. for 0.5 h. The mixture was cooled to RT and NaBH₃CN (8 mg, 0.12 mmol, 3.00 eq.) was added. The mixture was stirred at RT under N2 atmosphere overnight. The mixture was diluted with H₂O, extracted with EA, washed with brine, dried over Na₂SO₄, and concentrated, The residue was purified by prep-TLC (DCM:MeOH=20:1) to give the title compound (4.9 mg, 14.7%) as white solid. MS (ES, m/z): [M+1]⁺=793.3.

### Example 72

### Synthesis of 3-(4-(1-(1-(3-((4-((8-cyclopentyl-7-oxo-7,8-dihydropyrido[2,3-d]pyrimidin-2-yl)-amino)piperidin-1-yl)sulfonyl)phenyl)azetidin-3-yl)piperidin-4-yl)-3-methyl-2-oxo-2,3-dihydro-1H-benzo[d]imidazol-1-yl)piperidine-2,6-dione

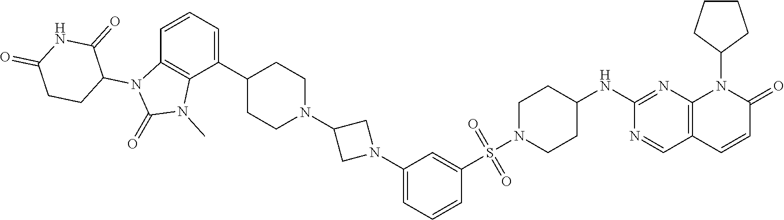 [SMILES: Cn1c(=O)n(C2CCC(=O)NC2=O)c2cccc(C3CCN(C4CN(c5cccc(S(=O)(=O)N6CCC(Nc7ncc8ccc(=O)n(C9CCCC9)c8n7)CC6)c5)C4)CC3)c21]

### Step 1: tert-butyl (1-((3-(3-(4-(1-(2,6-dioxopiperidin-3-yl)-3-methyl-2-oxo-2,3-dihydro-1H-benzo[d]imidazol-4-yl)piperidin-1-yl)azetidin-1-yl)phenyl)sulfonyl)piperidin-4-yl)carbamate

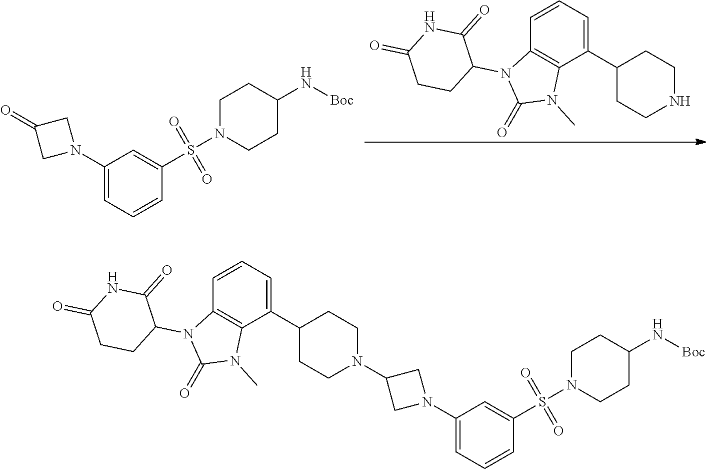 [SMILES: CC(C)(C)OC(=O)NC1CCN(S(=O)(=O)c2cccc(N3CC(=O)C3)c2)CC1 ; Cn1c(=O)n(C2CCC(=O)NC2=O)c2cccc(C3CCNCC3)c21 ; Cn1c(=O)n(C2CCC(=O)NC2=O)c2cccc(C3CCN(C4CN(c5cccc(S(=O)(=O)N6CCC(NC(=O)OC(C)(C)C)CC6)c5)C4)CC3)c21]

To a solution of 3-(3-methyl-2-oxo-4-(piperidin-4-yl)-2,3-dihydro-1H-benzo[d]imidazol-1-yl)piperidine-2,6-dione (41.00 mg, 0.12 mmol, 1.00 eq) in THF (2.00 mL) and DMF (0.50 mL) were added AcOH (3 drops) and tert-butyl (1-((3-(3-oxoazetidin-1-yl)phenyl)sulfonyl)piperidin-4-yl)carbamate (98.00 mg, 0.24 mmol, 2.00 eq.). The solution was stirred at 45° C. for 0.5 h. Then the solution cooled to RT and NaBH₃CN (15.08 mg, 0.24 mmol, 2.00 eq.) was added. The mixture was stirred at RT for 12 h and then diluted with water and extracted with EtOAc. The combined organic layer was washed with brine, dried over anhydrous Na₂SO₄ and concentrated. The residue was purified by TLC, eluted with DCM/MeOH (20:1), to afford the title compound (29.00 mg, 33.0%) as a white solid.

### Step 2: 3-(4-(1-(1-(3-((4-((8-cyclopentyl-7-oxo-7,8-dihydropyrido[2,3-d]pyrimidin-2-yl)-amino)piperidin-1-yl)sulfonyl)phenyl)azetidin-3-yl)piperidin-4-yl)-3-methyl-2-oxo-2,3-dihydro-1H-benzo[d]imidazol-1-yl)piperidine-2,6-dione

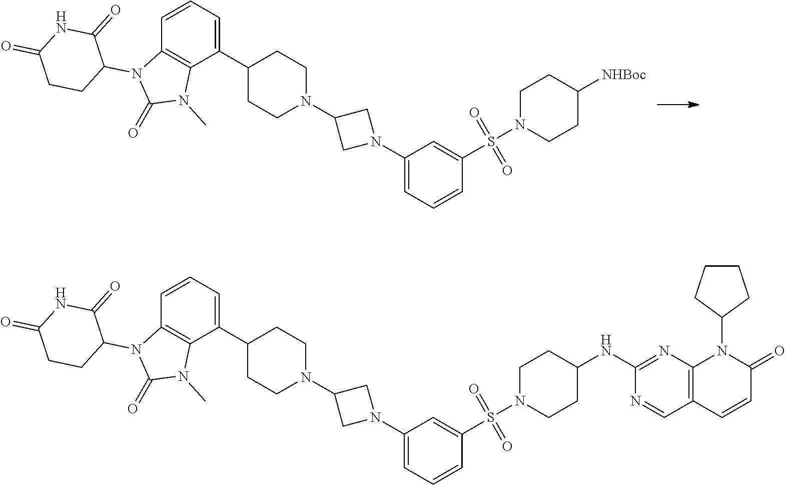 [SMILES: Cn1c(=O)n(C2CCC(=O)NC2=O)c2cccc(C3CCN(C4CN(c5cccc(S(=O)(=O)N6CCC(NC(=O)OC(C)(C)C)CC6)c5)C4)CC3)c21 ; Cn1c(=O)n(C2CCC(=O)NC2=O)c2cccc(C3CCN(C4CN(c5cccc(S(=O)(=O)N6CCC(Nc7ncc8ccc(=O)n(C9CCCC9)c8n7)CC6)c5)C4)CC3)c21]

tert-butyl (1-((3-(3-(4-(1-(2,6-Dioxopiperidin-3-yl)-3-methyl-2-oxo-2,3-dihydro-1H-benzo[d]imidazol-4-yl)piperidin-1-yl)azetidin-1-yl)phenyl)sulfonyl)piperidin-4-yl)carbamate was converted to the title compound by proceeding analogously as described in Example 7, Steps 5-6.

MS (ES, m/z): [M+1]⁺=849.

### Biological Examples

### Example 1

### NanoBRET CDK2 Engagement Assay

OVCAR3 cells with stable transfection of CDK2-Luciferase vector were seeded into 96-well plate at a density of 20,000 cells per well, 50 μL Opti-MEM medium supplemented with 1% FBS. Following morning, cells were treated with compounds of the disclosure, with starting concentration at 3 μM and ½ log dilution down to 0.3 nM for 24 hours at 37° C. DMSO treatment serves as control. Compound engagement was then measured using NanoBRE™ Kinase Kit from Promega (Part Number: CS1810C122, NanoBRET™ TE Kinase Kit #10, 1K) following manufacturer's instruction. For 24 hours treatment, tracer was added only for the last 2 hours. Briefly, 2.5 μL of complete 20× NanoBRET Tracer was added to the cells in suspension. Immediately prior to BRET measurements, prepare 3× Complete NanoBRET® Nano-Glo® Substrate in Opti-MEM without serum and phenol red. Without any delay, 25 μL per well of 3× Complete NanoBRET™ Nano-Glo® Substrate for a 96-well plate was added and mixed well. Signal was then read on a CLARIOstar® plate reader (measure donor emission (e.g., 450 nm) and acceptor emission (e.g., 610 nm or 630 nm) using a NanoBRET™-compatible luminometer. Target engagement was calculated with DMSO treatment as 100% signal, and IC₅₀ was calculated by GraphPad Prism 9.

### Example 2

### Inhibition of CDK2 and CDK1: Phospho-Rb Measurement in Cells

Phosphorylation of RB protein at S780 and S807/811 were measured using HTRF phospho-RB cellular kits (Cat #64RBS780PEG and 64RBS807PEG) from Cisbio.

On Day 1, OVCAR3 or KYSE520 cells were seeded into 96-well tissue-culture treated plates at 20,000 cells/well in 200 μL and incubated overnight at 37° C. in CO₂ atmosphere. On Day 2, the cells were treated with test compounds at concentrations from 0.3 to 10,000 nM using HP D300 digital dispenser. Twenty-four hours after compound treatment, cell culture media was removed by flicking the plate and tapping the plate against clean paper towel. Immediately 30 μL 1× lysis buffer was supplemented from the kit and the plate was incubated at room temperature on shaker for 30 min. After homogenization by pipetting up and down, 8 μL cell lysate from 96-well cell culture plate was transferred to 384-well small volume white detection plate. 2 μL premixed detection solution was added and the plate was covered with sealer. To prepare the detection solution, d2 conjugated-phospho-RB antibody and Eu-cryptate conjugated phosphor-RB antibody were diluted into detection buffer following manufacturer's instruction. Detection plates were incubated for 4 h at room temperature and read on ClarioStar (BMG Labtech) in TR-FRET mode (665 nM and 620 nM). The TR-FRET ratio (665 nM/620 nM) was plotted against the compound concentration and normalized to DMSO controls. Half maximal inhibition concentration (IC₅₀) values are calculated with a four-parameter logistic fit using GraphPad Prism (version 8; La Jolla, CA).

IC₅₀ of CDK2 PROTAC compounds in pRB(S807/811) assay are reported in Table 1 below.

TABLE 1

| Cpd No. | NanoBret | pRb OVCAR3 |
|---|---|---|
| (Cpd Table I) | (nM) | (nM) |
| 1 | 403.1 | 440.4 |
| 2 | 1310 | 1285 |
| 3 | 3042 | 2673 |
| 4 | 556.9 | 1240 |
| 5 | 4112 | 5858 |
| 6 | 1674 | 2247 |
| 7 | 99.9 | 1123 |
| 8 | 256.7 | 438.4 |
| 9 | 675.1 | 1808 |
| 10 | 790.8 | 3789 |
| 11 | 499.1 | 2061 |
| 12 | 4783 | 696.9 |
| 13 | 292.3 | 2120 |
| 14 | 668.3 | 633 |
| 15 | 356.4 | 2017 |
| 16 | 320.3 | 494.8 |
| 17 | 450 | 245.2 |
| 18 | 188.4 | 700.5 |
| 19 | 252.7 | 706.6 |
| 20 | 127.3 | 1174 |
| 21 | 1166 | 421.6 |
| 22 | 903.2 | 797.6 |
| 23 | <1 | 13.58 |
| 24 | 169 | 194.5 |
| 25 | | 1069 |
| 26 | | 6092 |
| 27 | 334.1 | 882.1 |
| 28 | 175.9 | 52.9 |
| 29 | 426.3 | 216.2 |
| 31 | 64 | 294 |
| 32 | 59 | 317 |
| 33 | | 312.9 |
| 34 | 85.97 | 50.27 |
| 35 | 139.3 | 71102 |
| 36 | 211.9 | 25.7 |
| 37 | 190.4 | 25.67 |
| 38 | 42.3 | 11.1 |
| 39 | 94 | 490 |
| 40 | 297.2 | 135.1 |
| 41 | 214.1 | 266.5 |
| 42 | 296.8 | 24.7 |
| 43 | 400.6 | 465 |
| 44 | n/a | 404.9 |
| 45 | 378.5 | 266.5 |
| 46 | 2164 | 2923 |
| 47 | 589.8 | 4031 |
| 48 | 722.4 | 131.2 |
| 49 | 272.3 | 835.6 |
| 50 | 289 | 49 |
| 51 | 198.1 | 154.2 |
| 52 | 499.2 | 1419 |
| 53 | 152 | 259.3 |
| 54 | 214.2 | 72.5 |
| 55 | 736 | 70 |
| 56 | 241 | 67 |
| 57 | 168.7 | 49.6 |
| 58 | 291.2 | 155.1 |
| 59 | 180.2 | 48.2 |
| 60 | 64.5 | 30 |
| 61 | 327.3 | 70.7 |
| 62 | 284 | 21.8 |
| 63 | 264.7 | 179.8 |
| 64 | 3042 | 7937 |
| 65 | 1750 | 6441 |
| 66 | 46 | 107 |
| 67 | 150 | 27 |
| 68 | 197.5 | 29.4 |
| 69 | 148.8 | 31.76 |
| 70 | 40.19 | 25.8 |
| 71 | 167.3 | 10.3 |
| 72 | 425.3 | 354.7 |
| 74 | | 100 |

It was also observed that, in general, the compounds in Table 1 above could inhibit CDK2 more selectively over CDK1, as indicated by more potent inhibition of pRb signaling for CDK2-dependent OVCAR3 cell line than for CDK2-independent but CDK1-dependent KYSE520 cell line. For example, compounds 1, 21, 28, 34, and 41 achieved about 9, 11, 21, 10.5 and 20.5 times more potent pRb inhibition in OVCAR3 than in KYSE520. Compound 5 was not tested in the KYSE520 cell line.

Selectivity over CDK1 is desired. Genetic studies in mice have demonstrated that while viable mice can develop from knockout of CDK2, CDK4 or CDK6, knocking out CDK1 did not yield viable homozygous mice or early stage embryos (see Santanmaria, et al. “Cdk1 is sufficient to drive the mammalian cell cycle.” Nature. 2007; 448:811-815; Satyanarayana and Kaldis, Oncogene 2009, 28, pages 2925-2939) and that CDK1 is required for cell cycle progression and it can functionally compensate for the loss of CDKs 2, 3, 4 and 6 by forming active complexes with cyclins D and E to drive the cell cycle (see Satyanarayana and Kaldis, 2009). Given that CDK1 is essential in cell proliferation, compounds that inhibit CDK1 may display toxicity that limits their clinical utility (see Brandeis, et al., “Cyclin B2-null mice develop normally and are fertile whereas cyclin B1-null mice die in utero.” Proc Natl Acad Sci USA. 1998; 95:4344-4349; Murphy, et al. “Delayed early embryonic lethality following disruption of the murine cyclin A2 gene.” Nat Genet. 1997; 15:83-86).

### Example 3

### High-Throughput Measurement of Cellular Endogenous CDK2

Effects of compounds on cellular CDK2 level can be monitored by a high-throughput HTRF assay or traditional Western Blot assay.

### A. CDK2 HTRF Assay

To determine half maximal degradation concentration (DC₅₀) values of compounds, cellular CDK2 level was measured in 96-well format using HTRF total CDK2 cellular kit (Cat #64CDK2TPEG) from Cisbio.

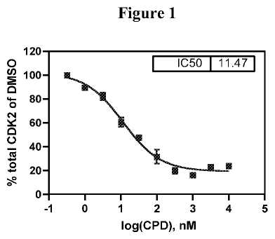

On Day 1, OVCAR3 cells were seeded into 96-well tissue-culture treated plates at 20,000 cells/well in 200 μL and incubated overnight at 37° C. in CO₂ atmosphere. On Day 2 cells were treated with compounds at concentration ranging from 0.3 to 10,000 nM using HP D300 digital dispenser. 24 hours after compound treatment, cell culture media was removed by flicking the plate and tapping the plate against clean paper towel. Immediately 30 μL 1× lysis buffer was supplemented from the kit and the plate was incubated at room temperature on shaker for 30 min. After homogenization by pipetting up and down, 8 μL cell lysate from 96-well cell culture plate was transferred to 384-well small volume white detection plate. 2 μL premixed detection solution was added and the plate was covered with sealer. To prepare the detection solution, d2 conjugated-CDK2 antibody and Eu-cryptate conjugated CDK2 antibody were diluted into detection buffer following manufacturer's instruction. Detection plates were incubated overnight at room temperature and read on ClarioStar (BMG Labtech) in TR-FRET mode (665 nM and 620 nM). The TR-FRET ratio (665 nM/620 nM) was plotted against the compound concentration and normalized to DMSO controls. Half maximal degradation concentration (DC₅₀) values were calculated with a four-parameter logistic fit using GraphPad Prism (version 8; La Jolla, CA). FIG. 1 provides a dose-response curve of Compound 1 in Compound Table I in cellular CDK2 HTRF assay. IC₅₀ in FIG. 1 is the same as DC₅₀. Compounds 1, 13, 16, 22, 23, 28, 34, 35, 36, 37, 38, 39, 42, 48, 50, 51, 53, 54, 55, 56, 57, 58, 59, 60, 61, 62, 63, 65, 66, 68, 69, 70 were tested and max degradation of CDK2 observed was from about 35% to about 90%.

### B. Western Blot Assay:

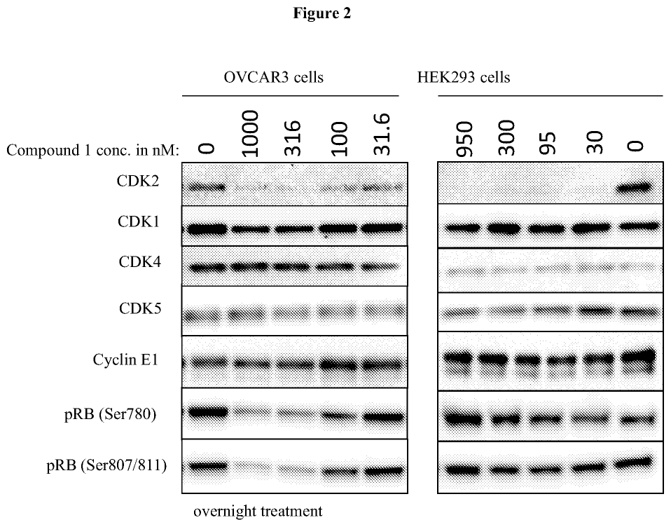

Standard Western Blot experiments were performed to monitor levels of proteins listed in FIG. 2 below, following treatment of OVCAR3 and HEK293 cells with Compound 1 in Compound Table 1.

OVCAR3 and HEK293 cells were seeded into 6-well plates at 0.4 million per well and incubated overnight at 37° C. in CO₂ atmosphere. Cells were treated with Compound 1 in Compound Table I for 20 h before collection. Cell lysates were made and subject to Western Blot analysis.

Results: As shown in FIG. 2, Compound 1 specifically induced reduction of CDK2 but had no effects on levels of CDK1, CDK4, CDK5 or cyclin E1 in both OVCAR3 and HEK293 cells. Loss of CDK2 blocked RB phosphorylation at S780 and S807/7811 was observed in CDK2-dependent OVCAR3 cells, but not in HEK293 cells.

All primary and secondary antibodies used were purchased from Cell Signaling Technologies.

| Antibody | Source | MW (kDa) | Vendor | Cat# |
|---|---|---|---|---|
| CDK1 | Rabbit | 34 | Cell Signaling | 28439 |
| CDK2 (78B2) | Rabbit | 33 | Cell Signaling | 2546 |
| CDK4 (D9G3E) | Rabbit | 30 | Cell Signaling | 12790 |
| CDK5 (D1F7M) | Rabbit | 30 | Cell Signaling | 14145 |
| Cyclin E1 (HE12) | Mouse | 50 | Cell Signaling | 4129 |
| Phospho-Rb | Rabbit | 110 | Cell Signaling | 8180 |
| (Ser780) (D59B7) | | | | |
| Phospho-Rb | Rabbit | 110 | Cell Signaling | 8516 |
| (Ser807/811) | | | | |
| (D20B12) | | | | |

### Formulation Examples

The following are representative pharmaceutical formulations containing a compound of the present disclosure.

### Tablet Formulation

The following ingredients are mixed intimately and pressed into single scored tablets.

| Ingredient | Quantity per tablet (mg) |
|---|---|
| compound Formula (I) | 400 |
| cornstarch | 50 |
| croscarmellose sodium | 25 |
| lactose | 120 |
| magnesium stearate | 5 |

### Capsule Formulation

The following ingredients are mixed intimately and loaded into a hard-shell gelatin capsule.

| Ingredient | Quantity per capsule (mg) |
|---|---|
| compound Formula (I) | 200 |
| lactose spray dried | 148 |
| magnesium stearate | 2 |

### Injectable Formulation

Compound of the disclosure (e.g., compound 1) in 2% HPMC, 1% Tween 80 in DI water, pH 2.2 with MSA, q.s. to at least 20 mg/mL

### Inhalation Composition

To prepare a pharmaceutical composition for inhalation delivery, 20 mg of a compound disclosed herein is mixed with 50 mg of anhydrous citric acid and 100 mL of 0.9% sodium chloride solution. The mixture is incorporated into an inhalation delivery unit, such as a nebulizer, which is suitable for inhalation administration.

### Topical Gel Composition

To prepare a pharmaceutical topical gel composition, 100 mg of a compound disclosed herein is mixed with 1.75 g of hydroxypropyl cellulose, 10 mL of propylene glycol, 10 mL of isopropyl myristate and 100 mL of purified alcohol USP. The resulting gel mixture is then incorporated into containers, such as tubes, which are suitable for topical administration.

### Ophthalmic Solution Composition

To prepare a pharmaceutical ophthalmic solution composition, 100 mg of a compound disclosed herein is mixed with 0.9 g of NaCl in 100 mL of purified water and filtered using a 0.2 micron filter. The resulting isotonic solution is then incorporated into ophthalmic delivery units, such as eye drop containers, which are suitable for ophthalmic administration.

### Nasal Spray Solution

To prepare a pharmaceutical nasal spray solution, 10 g of a compound disclosed herein is mixed with 30 mL of a 0.05M phosphate buffer solution (pH 4.4). The solution is placed in a nasal administrator designed to deliver 100 ul of spray for each application.

What is claimed:

1. A compound of Formula (IA′): 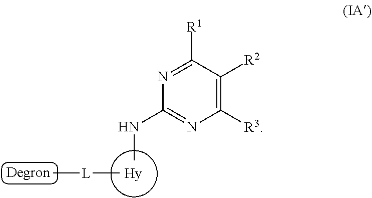
wherein:
Degron is an E3 ligase ligand of formula (i) or (ii); 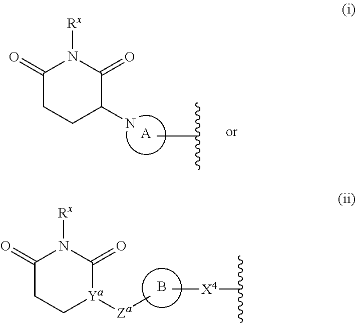
where:
R^{x} is hydrogen, alkyl, cycloalkyl, or alkylcarbonyloxy;
Y^{a} is CH or N;
Z^{a} is a bond, —CH₂—, —NH—, O, or —NHC(O)— where NH of —NHC(O)— is attached to Y^{a};
ring A is a group of formula (a), (b), or (c): 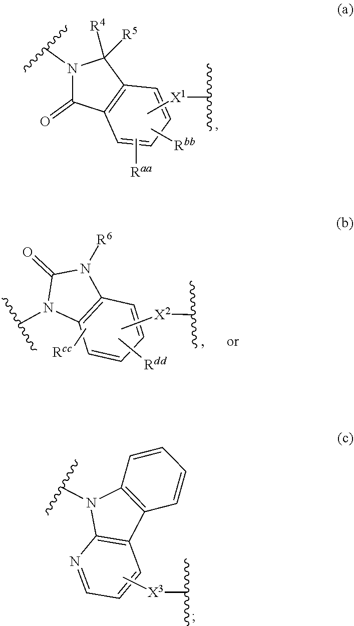
where:
R^{aa}, R^{bb}, R^{cc}, and R^{dd} are independently selected from hydrogen, alkyl, alkoxy, halo, haloalkyl, haloalkoxy, and cyano;
R⁴ and R⁵ are independently hydrogen or alkyl; or R⁴ and R⁵ together with the carbon to which they are attached form >C═O; and
R⁶ is hydrogen or alkyl;
ring B is phenylene, cyclylaminylene, a 5- or 6-membered monocyclic heteroarylene, or a 9- or 10-membered fused bicyclic heteroarylene, wherein each heteroarylene ring contains one to three nitrogen ring atoms and further wherein the phenylene, cyclylaminylene, and heteroarylene rings are independently substituted with R^{ee} and R^{ff} independently selected from hydrogen, alkyl, alkoxy, halo, haloalkyl, haloalkoxy, and cyano; and
X¹, X², X³ and X⁴ are independently a bond, -alkylene-, —O—, —(O-alkylene)-, -(alkylene-O)—, —(NR^{s}-alkylene)-, -(alkylene-NR^{t})—, —C≡C—, —NH—, —N(alkyl)-, —C(═O)—, —NR^{u}C(═O)—, or —C(═O)NR^{v}— where R^{s}, R^{t}, R^{u}, and R^{v} are independently hydrogen, alkyl, or cycloalkyl and each alkylene is optionally substituted with one or two fluoro; and
Hy is cycloalkylene, arylene, heterocyclylene, bicyclic heterocyclylene, spiro heterocyclylene, bridged heterocyclylene, or fused heterocyclylene, where each of the aforementioned rings is optionally substituted with one, two or three substituents independently selected from deuterium, alkyl, halo, haloalkyl, alkoxy, and hydroxy;
R¹ is hydrogen; and
R² and R³ together with the carbon atoms to which they are attached form a ring of formula (d1) 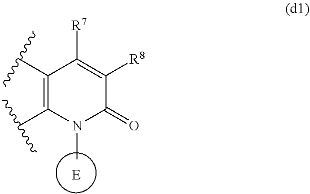
where:
R⁷ is hydrogen, alkyl, or haloalkyl;
R⁸ is hydrogen, cyano, halo, NH₂, alkyl, or haloalkyl where alkyl and haloalkyl are optionally substituted with R^{a} and R^{b} independently selected from hydroxy, cyano, alkoxy, haloalkoxy, C(O)NH₂, and —C(O)OH; and
ring E is bicyclic cycloalkyl, bridged cycloalkyl, or a ring of formula: 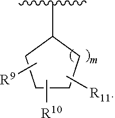
where m is 1, 2, or 3 and R⁹, R¹⁰, and R¹¹ are independently selected from hydrogen, deuterium, alkyl, haloalkyl, alkoxy, haloalkoxy, cyano, halo, and hydroxy; or when R⁹ and R¹⁰ are attached to the same carbon, R⁹ and R¹⁰ together with the carbon atom to which they are attached form cycloalkylene or heterocyclylene; and
L is —Z¹-Z²—Z³—Z⁴-Z⁵—Z⁶— where:
Z¹ is a bond, alkylene, —C(O)NR—, —NR′(CO)—, —S(O)₂NR—, —NR′S(O)₂—, —(O-alkylene)ₐ-, -(alkylene-O)ₐ—, phenylene, monocyclic heteroarylene, or heterocyclylene, where each ring is optionally substituted with one or two substituents independently selected from alkyl, alkoxy, halo, haloalkyl, and haloalkoxy;
Z² is a bond, alkylene, alkynylene, —C(O)—, —C(O)N(R)—, —NR′(CO)—, —(O-alkylene)_{b}-, -(alkylene-O)_{b}—, —O(CH₂)₇—, —O(CH₂)₃—, cycloalkylene, -heterocyclylene, where each ring is optionally substituted with one or two substituents independently selected from alkyl, alkoxy, halo, haloalkyl, and haloalkoxy;
Z³ is a bond, alkylene, alkynylene, —C(O)NR—, —NR′(CO)—, —O—, —NR″—, —(O-alkylene)_{c}-, -(alkylene-O)_{c}—, cycloalkylene, spiro cyclolalkylene, phenylene, monocyclic heteroarylene, heterocyclylene, bicyclic heterocyclylene, bridged heterocyclylene, fused heterocyclylene, spiro heterocyclylene, or 11 to 13 membered spiro heterocyclylene, where each ring is optionally substituted with one or two substituents independently selected from alkyl, alkoxy, halo, haloalkyl, and haloalkoxy;
Z⁴ is a bond, alkylene, alkynylene, -(alkylene-NR″)—, —O—, —C(O)—, —NR″—, —(O-alkylene)ₐ-, -(alkylene-O)_{d}—, cycloalkylene, spiro cyclolalkylene, phenylene, heteroarylene, heterocyclylene, fused heterocyclylene, bridged heterocyclylene, or spiro heterocyclylene, where each ring is optionally substituted with one or two substituents independently selected from alkyl, alkoxy, halo, haloalkyl, and haloalkoxy;
Z⁵ is a bond, -alkylene, —NR″—, —O—, —C(O)—, —S(O)₂—, —NR′(CO)—, —C(O)NR—, phenylene, monocyclic heteroarylene, or heterocycylene, where each ring is optionally substituted with one or two substituents independently selected from alkyl, alkoxy, halo, haloalkyl, and haloalkoxy, and
Z⁶ is a bond, alkylene, —NR″—, —O—, -(alkylene-O)—, —C(O)—, —S(O)₂—, —NR′(CO)—, or —C(O)NR—;
where each R, R′ and R″ is independently hydrogen or alkyl, each a, b, c, and d is independently an integer selected from 1 to 6 inclusive, and each alkylene is optionally substituted with one to four substituents where one, two, or three substituents are independently selected from fluoro and deuterium, and the fourth substituent is carboxy; provided that at least one of —Z¹-Z²—Z³—Z⁴-Z⁵—Z⁶— is not a bond; or
a pharmaceutically acceptable salt thereof.

2. The compound of claim 1 according to Formula (IA), or a pharmaceutically acceptable salt thereof: 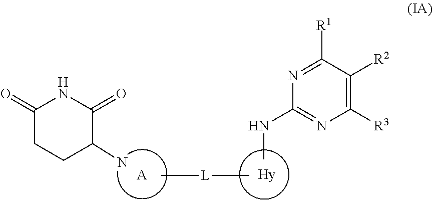
wherein:
ring A is a group of formula (a), (b), or (c): 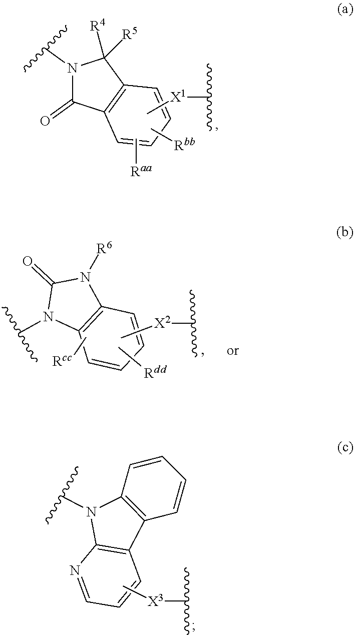
where:
R⁴ and R⁵ are independently hydrogen or alkyl; or R⁴ and R⁵ together with the carbon to which they are attached form >C═O;
R⁶ is hydrogen or alkyl; and
X¹, X², and X³ are independently a bond, -alkylene-, —O—, —(O-alkylene)-, -(alkylene-O)—, —(NR^{s}-alkylene)-, -(alkylene-NR^{t})—, —C≡C—, —NH—, —N(alkyl)-, —C(═O)—, —NR^{u}C(═O)—, or —C(═O)NR^{v}—where R^{s}, R^{t}, R^{u}, and R^{v} are independently hydrogen, alkyl, or cycloalkyl and each alkylene is optionally substituted with one or two fluoro;
Hy is cycloalkylene, arylene, heterocyclylene, bicyclic heterocyclylene, spiro heterocyclylene, bridged heterocyclylene, or fused heterocyclylene, where each of the aforementioned ring is optionally substituted with one or two substituents independently selected from hydrogen, alkyl, halo, haloalkyl, alkoxy, and hydroxy;
R¹ is hydrogen; and
R² and R³ together with the carbon atoms to which they are attached form a ring of formula (d): 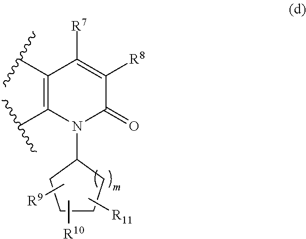
where:
m is 1, 2, or 3;
R⁷ is hydrogen, alkyl, or haloalkyl;
R⁸ is hydrogen, cyano, halo, NH₂, difluoromethyl, alkyl, or haloalkyl where alkyl and haloalkyl are substituted with R^{a} and R^{b} independently selected from hydroxy, cyano, alkoxy, haloalkoxy, C(O)NH₂, and —C(O)OH; and
R⁹, R¹⁰, and R¹¹ are independently selected from hydrogen, alkyl, haloalkyl, alkoxy, haloalkoxy, cyano, halo, and hydroxy; or
when R⁹ and R¹⁰ are attached to the same carbon, R⁹ and R¹⁰ together with the carbon atom to which they are attached can form cycloalkylene or heterocyclylene; and
L is —Z¹-Z²—Z³—Z⁴-Z⁵—Z⁶— where:
Z¹ is a bond, alkylene, —C(O)NR—, —NR′(CO)—, —S(O)₂NR—, —NR′S(O)₂—, —(O-alkylene)ₐ-, -(alkylene-O)ₐ—, phenylene, monocyclic heteroarylene, or heterocyclylene, where each ring is optionally substituted with one or two alkyl;
Z² is a bond, alkylene, alkynylene, —C(O)—, —C(O)N(R)—, —NR′(CO)—, —(O-alkylene)_{b}-, -(alkylene-O)_{b}—, —O(CH₂)₇—, —O(CH₂)₃—, cycloalkylene, or heterocyclylene, where each ring is optionally substituted with one or two alkyl;
Z³ is a bond, alkylene, alkynylene, —C(O)NR—, —NR′(CO)—, —O—, —NR″—, —(O-alkylene)ₑ-, -(alkylene-O)_{c}—, cycloalkylene, spiro cyclolalkylene, phenylene, monocyclic heteroarylene, heterocyclylene, bicyclic heterocyclylene, bridged heterocyclylene, fused heterocyclylene, spiro heterocyclylene, or 11 to 13 membered spiro heterocyclylene, where each ring is optionally substituted with one or two alkyl;
Z⁴ is a bond, alkylene, alkynylene, -(alkylene-NR″)—, —O—, —C(O)—, —NR″—, —(O-alkylene)ₐ-, -(alkylene-O)_{d}—, cycloalkylene, spiro cyclolalkylene, phenylene, heteroarylene, heterocyclylene, fused heterocyclylene, or spiro heterocyclylene, where each ring is optionally substituted with one or two alkyl;
Z⁵ is a bond, -alkylene, —NR″—, —O—, —C(O)—, —S(O)₂—, —NR′(CO)—, —C(O)NR—, phenylene, monocyclic heteroarylene, or heterocycylene, where each ring is optionally substituted with alkyl, and
Z⁶ is a bond, alkylene, —NR″—, —O—, -(alkylene-O)—, —C(O)—, —S(O)₂—, —NR′(CO)—, or —C(O)NR—;
where each R, R′ and R″ is independently hydrogen or alkyl, each a, b, c, and d is independently an integer selected from 1 to 6 inclusive, and each alkylene is optionally substituted with one, two, or three fluoro or a carboxy; provided that at least one of —Z¹-Z²—Z³—Z⁴-Z⁵—Z⁶— is not a bond.

3. The compound of claim 1, or a pharmaceutically acceptable salt thereof, wherein:
X¹, X², X³, X⁴, Z¹, and Z² are each a bond;
Z³ is cycloalkylene, phenylene, monocyclic heteroarylene, heterocyclylene, bicyclic heterocyclylene, bridged heterocyclylene, fused heterocyclylene, or spiro heterocyclylene, where each ring is optionally substituted with one or two substituents independently selected from alkyl, alkoxy, halo, haloalkyl, and haloalkoxy;
Z⁴ is a bond, alkylene, —O—, cycloalkylene, phenylene, monocyclic heteroarylene, heterocyclylene, fused heterocyclylene, or spiro heterocyclylene, where each ring is optionally substituted with one or two substituents independently selected from alkyl, alkoxy, halo, haloalkyl, and haloalkoxy;
Z⁵ is phenylene, monocyclic heteroarylene, or heterocycylene, where each ring is optionally substituted with one or two substituents independently selected from alkyl, alkoxy, halo, haloalkyl, and haloalkoxy;
Z⁶ is —S(O)₂—; and
wherein each alkylene is optionally substituted with one, two, or three deuterium.

4. The compound of claim 3, or a pharmaceutically acceptable salt thereof, wherein:
Z³ is heterocyclylene, bicyclic heterocyclylene, bridged heterocyclylene, fused heterocyclylene, or spiro heterocyclylene, where each ring is optionally substituted with one or two substituents independently selected from alkyl, alkoxy, halo, haloalkyl, and haloalkoxy; and
Z⁴ is alkylene, —O—, monocyclic heteroarylene, heterocyclylene, fused heterocyclylene, or spiro heterocyclylene, where each ring is optionally substituted with one or two substituents independently selected from alkyl, alkoxy, halo, haloalkyl, and haloalkoxy.

5. The compound of claim 3 or 4, or a pharmaceutically acceptable salt thereof,
wherein:
Z³ is heterocyclylene, bridged heterocyclylene, or spiro heterocyclylene, where each ring is optionally substituted with one or two substituents independently selected from alkyl, alkoxy, halo, haloalkyl, and haloalkoxy;
Z⁴ is alkylene, —O—, or heterocyclylene, where each ring is optionally substituted with one or two substituents independently selected from alkyl, alkoxy, halo, haloalkyl, and haloalkoxy;
Z⁵ is phenylene, monocyclic heteroarylene, or heterocycylene, where each ring is optionally substituted with one or two substituents independently selected from alkyl, alkoxy, halo, haloalkyl, and haloalkoxy.

6. The compound of any one of claims 3 to 5, or a pharmaceutically acceptable salt
thereof, wherein:
Z³ is heterocyclylene, bridged heterocyclylene, or spiro heterocyclylene, where each ring is optionally substituted with one or two substituents independently selected from alkyl, alkoxy, halo, haloalkyl, and haloalkoxy;
Z⁴ is alkylene, —O—, or heterocyclylene, where each ring is optionally substituted with one or two substituents independently selected from alkyl, alkoxy, halo, haloalkyl, and haloalkoxy; and
Z⁵ is phenylene optionally substituted with one or two substituents independently selected from alkyl, alkoxy, halo, haloalkyl, and haloalkoxy.

7. The compound of claim 1, or a pharmaceutically acceptable salt thereof, wherein:
X¹, X², X³, X⁴, and Z¹ are each a bond;
Z² is cycloalkylene or heterocyclylene, where each ring is optionally substituted with one or two substituents independently selected from alkyl, alkoxy, halo, haloalkyl, and haloalkoxy;
Z³ is cycloalkylene, phenylene, monocyclic heteroarylene, heterocyclylene, bicyclic heterocyclylene, bridged heterocyclylene, fused heterocyclylene, or spiro heterocyclylene, where each ring is optionally substituted with one or two substituents independently selected from alkyl, alkoxy, halo, haloalkyl, and haloalkoxy;
Z⁴ is a bond, alkylene, or —O—;
Z⁵ is phenylene, monocyclic heteroarylene (e.g., pyridindiyl), or heterocycylene, where each ring is optionally substituted with one or two substituents independently selected from alkyl, alkoxy, halo, haloalkyl, and haloalkoxy;
Z⁶ is —S(O)₂—; and
wherein each alkylene is optionally substituted with one, two, or three deuterium.

8. The compound of claim 7, or a pharmaceutically acceptable salt thereof, wherein:
Z² is heterocyclylene, where each ring is optionally substituted with one or two substituents independently selected from alkyl, alkoxy, halo, haloalkyl, and haloalkoxy;
Z³ is heterocyclylene optionally substituted with one or two substituents independently selected from alkyl, alkoxy, halo, haloalkyl, and haloalkoxy;
Z⁴ is a bond, alkylene, or —O—; and
Z⁵ is phenylene optionally substituted with one or two substituents independently selected from alkyl, alkoxy, halo, haloalkyl, and haloalkoxy.

9. The compound of claim 1, or a pharmaceutically acceptable salt thereof, wherein:
X¹, X², X³, and X⁴, and Z¹ are each a bond;
Z² is heterocyclylene, where each ring is optionally substituted with one or two substituents independently selected from alkyl, alkoxy, halo, haloalkyl, and haloalkoxy;
Z³ is a bond, alkylene, or —O—;
Z⁴ is heterocyclylene, bridged heterocyclylene, or spiro heterocyclylene, where each ring is optionally substituted with one or two substituents independently selected from alkyl, alkoxy, halo, haloalkyl, and haloalkoxy;
Z⁵ is phenylene optionally substituted with one or two substituents independently selected from alkyl, alkoxy, halo, haloalkyl, and haloalkoxy;
Z⁶ is —S(O)₂—; and
wherein each alkylene is optionally substituted with one, two, or three deuterium.

10. The compound of any one of claims 1 to 9, or a pharmaceutically acceptable salt thereof, wherein —Z⁵— is 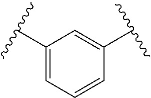 [SMILES: *c1cccc(*)c1]
optionally substituted with one or two substituents independently selected from alkyl, alkoxy, halo, haloalkyl, and haloalkoxy.

11. The compound of claim 1, or a pharmaceutically acceptable salt thereof, wherein —X¹-L-, —X²-L-, —X³-L-, and —X⁴-L- are independently selected from:

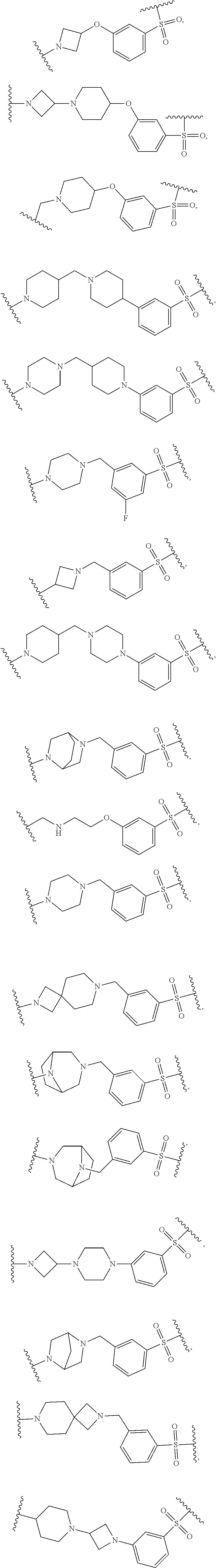

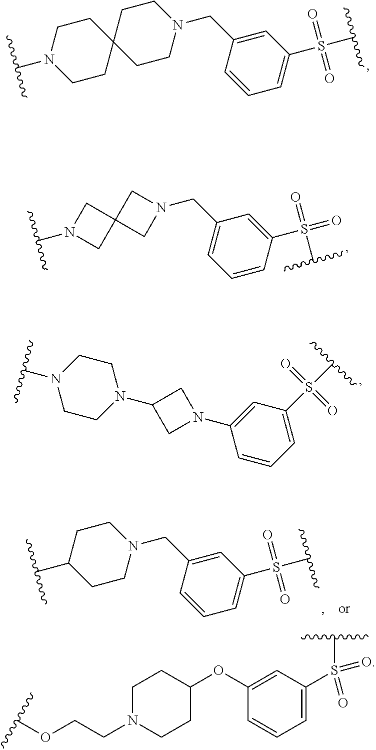 [SMILES: *N1CCC2(CC1)CCN(Cc1cccc(S(*)(=O)=O)c1)CC2 ; *N1CC2(C1)CN(Cc1cccc(S(*)(=O)=O)c1)C2 ; *N1CCN(C2CN(c3cccc(S(*)(=O)=O)c3)C2)CC1 ; *C1CCN(Cc2cccc(S(*)(=O)=O)c2)CC1 ; *OCCN1CCC(Oc2cccc(S(*)(=O)=O)c2)CC1]

12. The compound of any one of claims 1 to 11, or a pharmaceutically acceptable salt thereof, where ring E is a ring of formula

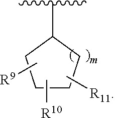

13. The compound of any one of claims 1 to 12, or a pharmaceutically acceptable salt thereof, wherein R⁹, R¹⁰, and R¹¹ are each independently hydrogen or deuterium.

14. The compound of any one of claims 1 to 12, or a pharmaceutically acceptable salt thereof, wherein R⁹, R¹⁰, and R¹¹ are independently selected from hydrogen, deuterium, alkyl, haloalkyl, alkoxy, haloalkoxy, cyano, halo, and hydroxy.

15. The compound of claim any one of claims 1 to 12, or a pharmaceutically acceptable salt thereof, wherein R⁹ is hydrogen or deuterium, R¹⁰ is hydrogen or alkyl, and R″ hydrogen or hydroxy.

16. The compound of any one of claims 1 to 15, or a pharmaceutically acceptable salt thereof, is wherein m is 1.

17. The compound of claim 1 or 12, or a pharmaceutically acceptable salt thereof, wherein ring of formula 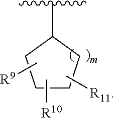
is a group of structure:

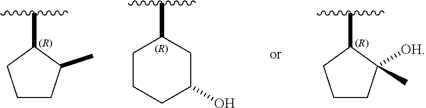

18. The compound of any one of claims 1 and 3 to 11, or a pharmaceutically acceptable salt thereof, wherein ring E is bridged cycloalkyl or bicyclic cycloalkyl.

19. The compound of any one of claims 1 to 18, or a pharmaceutically acceptable salt thereof, wherein R⁸ is hydrogen, halo, haloalkyl, or alkyl (optionally substituted with hydroxy).

20. The compound of any one of claims 1 to 18, or a pharmaceutically acceptable salt thereof, wherein R⁸ is hydrogen.

21. The compound of any one of claims 1 to 18, or a pharmaceutically acceptable salt thereof, wherein R⁸ is haloalkyl.

22. The compound of claim 21, or a pharmaceutically acceptable salt thereof, wherein R⁸ is difluoromethyl.

23. The compound of claim 1 to 18, or a pharmaceutically acceptable salt thereof, wherein R⁸ is 2-hydroxymethyl.

24. The compound of any one of claims 1 to 23, or a pharmaceutically acceptable salt thereof, wherein R⁷ is hydrogen.

25. The compound of any one of claims 1 and 3 to 24, or a pharmaceutically acceptable salt thereof, wherein Hy is heterocyclylene optionally substituted with one or two substituents independently selected from deuterium, alkyl, halo, haloalkyl, alkoxy, and hydroxy.

26. The compound of any one of claims 1 to 24, or a pharmaceutically acceptable salt thereof, wherein Hy is piperidin-1,4-diyl and L is attached to the nitrogen atom of the piperidin-1,4-diyl ring of Hy.

27. The compound of any one of claims 1 to 24, or a pharmaceutically acceptable salt thereof, wherein Hy is 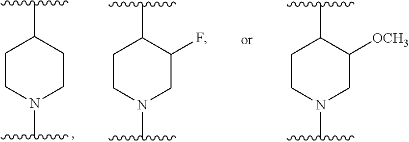 [SMILES: *C1CCN(*)CC1 ; *C1CCN(*)CC1F ; *C1CCN(*)CC1OC]
where the N atom of the piperidine ring is attached to L.

28. The compound of any one of claims 1 and 3 to 24, or a pharmaceutically acceptable salt thereof, wherein Hy is phenylene optionally substituted with one, or two substituents independently selected from alkyl, halo, haloalkyl, alkoxy, and hydroxy.

29. The compound of any one of claims 1 and 3 to 24, or a pharmaceutically acceptable salt thereof, wherein Hy is spiro heterocyclylene optionally substituted with one or two substituents independently selected from alkyl, halo, haloalkyl, alkoxy, and hydroxy.

30. The compound of any one of claims 1 to 24, or a pharmaceutically acceptable salt thereof, wherein Hy is bridged heterocyclylene optionally substituted with one or two substituents independently selected from alkyl, halo, haloalkyl, alkoxy, and hydroxy.

31. The compound of any one of claims 1 and 3 to 30, or a pharmaceutically acceptable salt thereof, wherein the Degron is an E3 ligase ligand of formula (i):

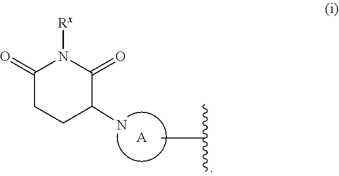

32. The compound of any one of claims 1 and 3 to 31, or a pharmaceutically acceptable salt thereof, wherein the ring A of the E3 ligase ligand of formula (i) is:

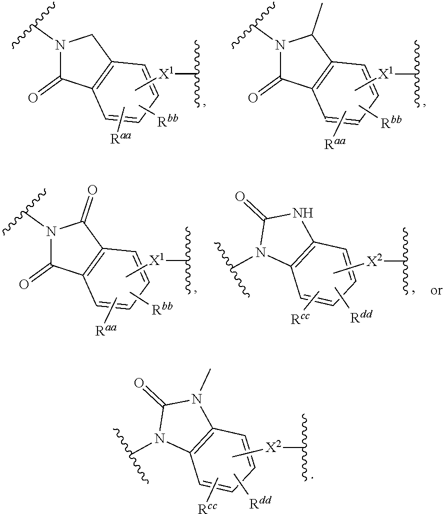

33. The compound of any one of claims 1 and 3 to 31, or a pharmaceutically acceptable salt thereof, wherein the ring A of the E3 ligase ligand of formula (i) is:

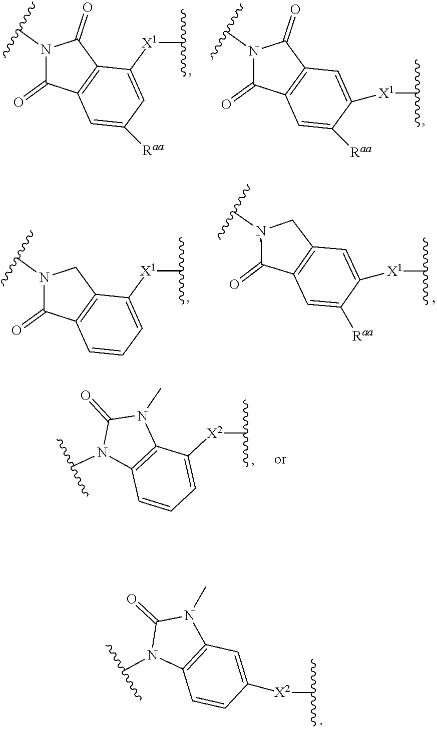

34. The compound of any one of claims 1 and 3 to 33, or a pharmaceutically acceptable salt thereof, wherein R^{aa}, R^{bb}, R^{cc} and R^{dd} are independently selected from hydrogen, methyl, methoxy, ethoxy, fluoro, trifluoromethyl, difluoromethyl, and trifluoromethoxy.

35. The compound of any one of claims 1 and 3 to 30, or a pharmaceutically acceptable salt thereof, wherein the Degron is an E3 ligase ligand of formula (ii):

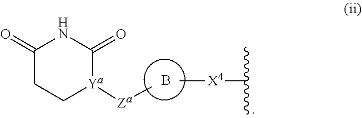

36. The compound of any one of claims 1, 3 to 30, and 35, or a pharmaceutically acceptable salt thereof, wherein the E3 ligase ligand of formula (ii) is: 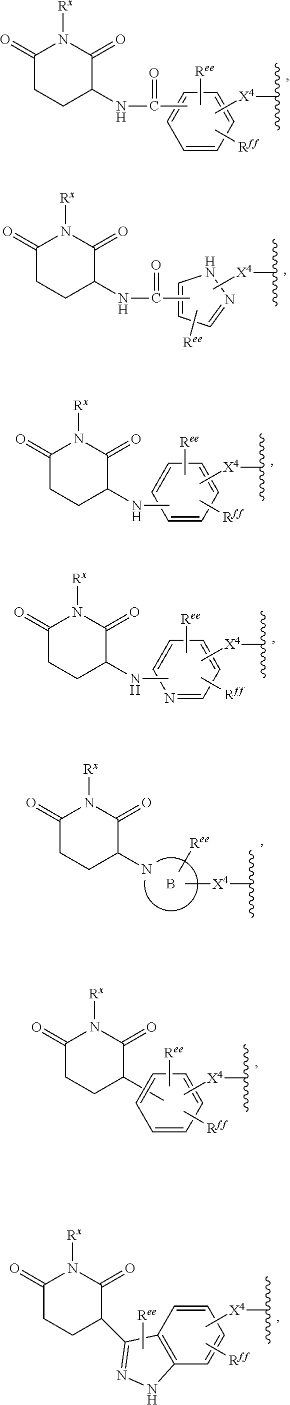 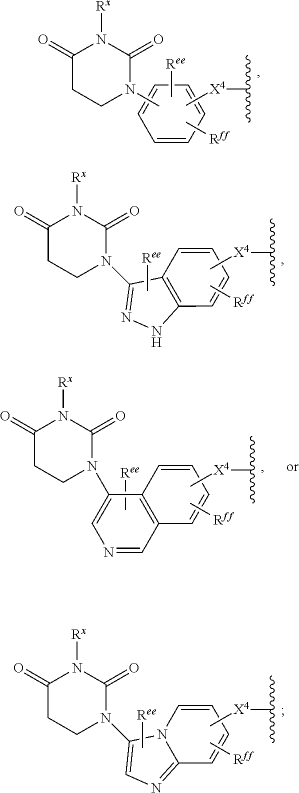
where ring B is cyclylaminylene.

37. The compound ofany one of claims 1, 3 to 30, 35 and 36, or a pharmaceutically acceptable salt thereof, wherein the E3 ligase ligand of formula (ii) is:

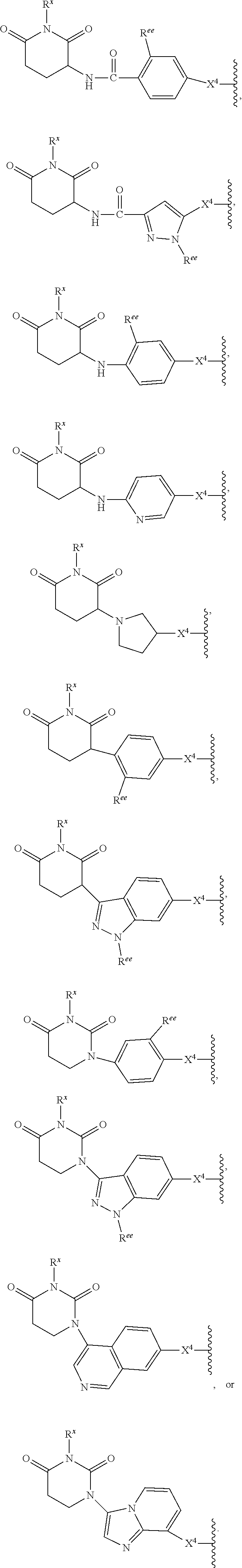

38. The compound of any one of claims 1, 3 to 30, 35 and 36, or a pharmaceutically acceptable salt thereof, wherein the E3 ligase ligand of formula (ii) is:

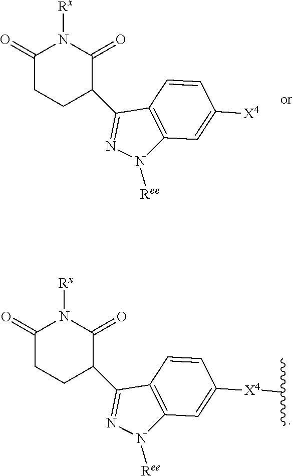

39. The compound of any one of claims 1, 3 to 30 and 35 to 38, or a pharmaceutically acceptable salt thereof, wherein R^{ee} and R^{ff} are independently selected from hydrogen, methyl, methoxy, ethoxy, fluoro, chloro, trifluoromethyl, difluoromethyl, and trifluoromethoxy.

40. The compound of any one of claims 1-39, or a pharmaceutically acceptable salt thereof, wherein R^{x} is hydrogen.

41. A compound selected from:
4-((8-cyclopentyl-7-oxo-7,8-dihydropyrido[2,3-d]pyrimidin-2-yl)amino)-N-(14-((2-(2,6-
dioxopiperidin-3-yl)-1,3-dioxoisoindolin-4-yl)amino)-3,6,9,12-
tetraoxatetradecyl)piperidine-1-sulfonamide
N-(2-(2-(2-(4-((4-((8-cyclopentyl-7-oxo-7,8-dihydropyrido[2,3-d]pyrimidin-2-
yl)amino)piperidin-1-yl)sulfonyl)piperazin-1-yl)ethoxy)ethoxy)ethyl)-2-((2-(2,6-
dioxopiperidin-3-yl)-1,3-dioxoisoindolin-4-yl)oxy)acetamide
N-(2-(2-(4-((4-((8-cyclopentyl-7-oxo-7,8-dihydropyrido[2,3-d]pyrimidin-2-
yl)amino)piperidin-1-yl)sulfonyl)piperazin-1-yl)ethoxy)ethyl)-2-((2-(2,6-dioxopiperidin-3-
yl)-1,3-dioxoisoindolin-4-yl)oxy)acetamide
N-(2-(2-(2-(2-(4-((4-((8-cyclopentyl-7-oxo-7,8-dihydropyrido[2,3-d]pyrimidin-2-
yl)amino)piperidin-1-yl)sulfonyl)piperazin-1-yl)ethoxy)ethoxy)ethoxy)ethyl)-2-((2-(2,6-
dioxopiperidin-3-yl)-1,3-dioxoisoindolin-4-yl)oxy)acetamide
N-(2-(2-(4-((4-((8-cyclopentyl-7-oxo-7,8-dihydropyrido[2,3-d]pyrimidin-2-
yl)amino)piperidin-1-yl)sulfonyl)piperazin-1-yl)ethoxy)ethyl)-2-((2-(1-methyl-2,6-
dioxopiperidin-3-yl)-1,3-dioxoisoindolin-4-yl)oxy)acetamide
N-(14-(4-((4-((8-cyclopentyl-7-oxo-7,8-dihydropyrido[2,3-d]pyrimidin-2-
yl)amino)piperidin-1-yl)sulfonyl)piperazin-1-yl)-3,6,9,12-tetraoxatetradecyl)-2-((2-(2,6-
dioxopiperidin-3-yl)-1,3-dioxoisoindolin-4-yl)oxy)acetamide
5-(3-(4-((4-((8-cyclopentyl-7-oxo-7,8-dihydropyrido[2,3-d]pyrimidin-2-
yl)amino)piperidin-1-yl)sulfonyl)phenoxy)azetidin-1-yl)-2-(2,6-dioxopiperidin-3-
yl)isoindoline-1,3-dione
5-((3-(4-((4-((8-cyclopentyl-7-oxo-7,8-dihydropyrido[2,3-d]pyrimidin-2-
yl)amino)piperidin-1-yl)sulfonyl)phenoxy)azetidin-1-yl)methyl)-2-(2,6-dioxopiperidin-3-
yl)isoindoline-1,3-dione
4-((8-cyclopentyl-7-oxo-7,8-dihydropyrido[2,3-d]pyrimidin-2-yl)amino)-N-(2-(2-(2-((2-
(2,6-dioxopiperidin-3-yl)-1,3-dioxoisoindolin-4-yl)amino)ethoxy)ethoxy)ethyl)piperidine-
1-sulfonamide
4-((8-cyclopentyl-7-oxo-7,8-dihydropyrido[2,3-d]pyrimidin-2-yl)amino)-N-(3-(3-(2-(2,6-
dioxopiperidin-3-yl)-1,3-dioxoisoindolin-4-yl)propoxy)propyl)-N-methylpiperidine-1-
sulfonamide
4-((8-cyclopentyl-7-oxo-7,8-dihydropyrido[2,3-d]pyrimidin-2-yl)amino)-N-(2-(2-(3-(2-
(2,6-dioxopiperidin-3-yl)-1,3-dioxoisoindolin-4-yl)propoxy)ethoxy)ethyl)piperidine-1-
sulfonamide
5-(3-(3-((4-((8-cyclopentyl-7-oxo-7,8-dihydropyrido[2,3-d]pyrimidin-2-
yl)amino)piperidin-1-yl)sulfonyl)phenoxy)azetidin-1-yl)-2-(2,6-dioxopiperidin-3-
yl)isoindoline-1,3-dione
3-(4-(3-((1-((1-((4-((8-cyclopentyl-7-oxo-7,8-dihydropyrido[2,3-d]pyrimidin-2-
yl)amino)piperidin-1-yl)sulfonyl)piperidin-4-yl)methyl)piperidin-4-yl)oxy)prop-1-yn-1-
yl)-3-methyl-2-oxo-2,3-dihydro-1H-benzo[d]imidazol-1-yl)piperidine-2,6-dione
3-(4-(3-((1-((4-((8-cyclopentyl-7-oxo-7,8-dihydropyrido[2,3-d]pyrimidin-2-
yl)amino)piperidin-1-yl)sulfonyl)piperidin-4-yl)oxy)prop-1-yn-1-yl)-3-methyl-2-oxo-2,3-
dihydro-1H-benzo[d]imidazol-1-yl)piperidine-2,6-dione
4-((8-cyclopentyl-7-oxo-7,8-dihydropyrido[2,3-d]pyrimidin-2-yl)amino)-N-(4-((2-(2,6-
dioxopiperidin-3-yl)-1,3-dioxoisoindolin-4-yl)amino)cyclohexyl)-N-methylpiperidine-1-
sulfonamide
3-(4-(3-((1-((4-((8-cyclopentyl-7-oxo-7,8-dihydropyrido[2,3-d]pyrimidin-2-
yl)amino)piperidin-1-yl)sulfonyl)piperidin-4-yl)oxy)propyl)-3-methyl-2-oxo-2,3-dihydro-
1H-benzo[d]imidazol-1-yl)piperidine-2,6-dione
5-((3-(3-((4-((8-cyclopentyl-7-oxo-7,8-dihydropyrido[2,3-d]pyrimidin-2-
yl)amino)piperidin-1-yl)sulfonyl)phenoxy)azetidin-1-yl)methyl)-2-(2,6-dioxopiperidin-3-
yl)isoindoline-1,3-dione
4-((8-cyclopentyl-7-oxo-7,8-dihydropyrido[2,3-d]pyrimidin-2-yl)amino)-N-(2-(3-(1-(2,6-
dioxopiperidin-3-yl)-3-methyl-2-oxo-2,3-dihydro-1H-benzo[d]imidazol-4-
yl)propoxy)ethyl)piperidine-1-sulfonamide
4-((8-cyclopentyl-7-oxo-7,8-dihydropyrido[2,3-d]pyrimidin-2-yl)amino)-N-(3-(3-(1-(2,6-
dioxopiperidin-3-yl)-3-methyl-2-oxo-2,3-dihydro-1H-benzo[d]imidazol-4-
yl)propoxy)propyl)-N-methylpiperidine-1-sulfonamide
4-((8-cyclopentyl-7-oxo-7,8-dihydropyrido[2,3-d]pyrimidin-2-yl)amino)-N-(1-((1-(2,6-
dioxopiperidin-3-yl)-3-methyl-2-oxo-2,3-dihydro-1H-benzo[d]imidazol-4-
yl)methyl)piperidin-4-yl)-N-methylpiperidine-1-sulfonamide
4-((8-cyclopentyl-7-oxo-7,8-dihydropyrido[2,3-d]pyrimidin-2-yl)amino)-N-(2-(2-(2-(2-
((2-(2,6-dioxopiperidin-3-yl)-1,3-dioxoisoindolin-4-
yl)amino)ethoxy)ethoxy)ethoxy)ethyl)piperidine-1-sulfonamide
4-((8-cyclopentyl-7-oxo-7,8-dihydropyrido[2,3-d]pyrimidin-2-yl)amino)-N-(2-((4-((2-(2,6-
dioxopiperidin-3-yl)-1,3-dioxoisoindolin-4-
yl)(methyl)amino)benzyl)(methyl)amino)ethyl)-N-methylpiperidine-1-sulfonamide
4-((8-cyclopentyl-7-oxo-7,8-dihydropyrido[2,3-d]pyrimidin-2-yl)amino)-N-(3-(4-(1-(2,6-
dioxopiperidin-3-yl)-3-methyl-2-oxo-2,3-dihydro-1H-benzo[d]imidazol-4-yl)piperidin-1-
yl)propyl)-N-methylpiperidine-1-sulfonamide
4-((8-cyclopentyl-7-oxo-7,8-dihydropyrido[2,3-d]pyrimidin-2-yl)amino)-N-(((2R)-4-(3-(1-
(2,6-dioxopiperidin-3-yl)-3-methyl-2-oxo-2,3-dihydro-1H-benzo[d]imidazol-4-
yl)propyl)morpholin-2-yl)methyl)-N-methylpiperidine-1-sulfonamide
4-((15-((4-((8-cyclopentyl-7-oxo-7,8-dihydropyrido[2,3-d]pyrimidin-2-yl)amino)piperidin-
1-yl)sulfonyl)-3,6,9-trioxa-12-azapentadecyl)amino)-2-(2,6-dioxopiperidin-3-
yl)isoindoline-1,3-dione
14-((4-((8-cyclopentyl-7-oxo-7,8-dihydropyrido[2,3-d]pyrimidin-2-yl)amino)piperidine)-
1-sulfonamido)-N-(2-(2,6-dioxopiperidin-3-yl)-1,3-dioxoisoindolin-4-yl)-3,6,9,12-
tetraoxatetradecanamide
3-(4-(2-(3-(3-((4-((8-cyclopentyl-7-oxo-7,8-dihydropyrido[2,3-d]pyrimidin-2-
yl)amino)piperidin-1-yl)sulfonyl)phenoxy)azetidin-1-yl)ethoxy)-1-oxoisoindolin-2-
yl)piperidine-2,6-dione
3-(5-(3-(4-(3-((4-((8-cyclopentyl-7-oxo-7,8-dihydropyrido[2,3-d]pyrimidin-2-
yl)amino)piperidin-1-yl)sulfonyl)phenoxy)piperidin-1-yl)azetidin-1-yl)-1-oxoisoindolin-2-
yl)piperidine-2,6-dione
3-(4-(2-(4-(3-((4-((8-cyclopentyl-7-oxo-7,8-dihydropyrido[2,3-d]pyrimidin-2-
yl)amino)piperidin-1-yl)sulfonyl)phenoxy)piperidin-1-yl)ethoxy)-1-oxoisoindolin-2-
yl)piperidine-2,6-dione
3-(4-(1-(3-((4-((8-cyclopentyl-7-oxo-7,8-dihydropyrido[2,3-d]pyrimidin-2-
yl)amino)piperidin-1-yl)sulfonyl)benzyl)piperidin-4-yl)-3-methyl-2-oxo-2,3-dihydro-1H-
benzo[d]imidazol-1-yl)piperidine-2,6-dione
3-(4-(1′-((4-((8-cyclopentyl-7-oxo-7,8-dihydropyrido[2,3-d]pyrimidin-2-
yl)amino)piperidin-1-yl)sulfonyl)-[1,4′-bipiperidin]-4-yl)-3-methyl-2-oxo-2,3-dihydro-1H-
benzo[d]imidazol-1-yl)piperidine-2,6-dione
3-(4-(1-(2-((4-((8-cyclopentyl-7-oxo-7,8-dihydropyrido[2,3-d]pyrimidin-2-
yl)amino)piperidin-1-yl)sulfonyl)-2-azaspiro[3.3]heptan-6-yl)piperidin-4-yl)-3-methyl-2-
oxo-2,3-dihydro-1H-benzo[d]imidazol-1-yl)piperidine-2,6-dione
3-(5-(4-(3-((4-((8-cyclopentyl-7-oxo-7,8-dihydropyrido[2,3-d]pyrimidin-2-
yl)amino)piperidin-1-yl)sulfonyl)phenoxy)piperidin-1-yl)-1-oxoisoindolin-2-yl)piperidine-
2,6-dione
3-(5-(4-((4-(3-((4-((8-cyclopentyl-7-oxo-7,8-dihydropyrido[2,3-d] pyrimidin-2-
yl)amino)piperidin-1-yl)sulfonyl)phenyl)piperidin-1-yl)methyl)piperidin-1-yl)-1-
oxoisoindolin-2-yl)piperidine-2,6-dione
3-(5-(1-((1-(3-((4-((8-cyclopentyl-7-oxo-7,8-dihydropyrido[2,3-d]pyrimidin-2-
yl)amino)piperidin-1-yl)sulfonyl)phenyl)piperidin-4-yl)methyl)piperidin-4-yl)-1-
oxoisoindolin-2-yl)piperidine-2,6-dione
3-(5-(4-((1-(3-((4-((8-cyclopentyl-7-oxo-7,8-dihydropyrido[2,3-d]pyrimidin-2-
yl)amino)piperidin-1-yl)sulfonyl)phenyl)piperidin-4-yl)methyl)piperazin-1-yl)-1-
oxoisoindolin-2-yl)piperidine-2,6-dione
3-(5-(1-(3-((4-((8-cyclopentyl-7-oxo-7,8-dihydropyrido[2,3-d]pyrimidin-2-
yl)amino)piperidin-1-yl)sulfonyl)benzyl)azetidin-3-yl)-1-oxoisoindolin-2-yl)piperidine-
2,6-dione
3-(5-(4-((4-(3-((4-((8-cyclopentyl-7-oxo-7,8-dihydropyrido[2,3-d]pyrimidin-2-
yl)amino)piperidin-1-yl)sulfonyl)phenyl)piperazin-1-yl)methyl)piperidin-1-yl)-1-
oxoisoindolin-2-yl)piperidine-2,6-dione
3-(4-(1′-(3-((4-((8-cyclopentyl-7-oxo-7,8-dihydropyrido[2,3-d]pyrimidin-2-
yl)amino)piperidin-1-yl)sulfonyl)phenyl)-[1,4′-bipiperidin]-4-yl)-3-methyl-2-oxo-2,3-
dihydro-1H-benzo[d]imidazol-1-yl)piperidine-2,6-dione
3-(4-(1-(3-((4-((8-cyclopentyl-7-oxo-7,8-dihydropyrido[2,3-d]pyrimidin-2-
yl)amino)piperidin-1-yl)sulfonyl)benzyl)azetidin-3-yl)-3-methyl-2-oxo-2,3-dihydro-1H-
benzo[d]imidazol-1-yl)piperidine-2,6-dione
3-(4-(((2-(3-((4-((8-cyclopentyl-7-oxo-7,8-dihydropyrido[2,3-d]pyrimidin-2-
yl)amino)piperidin-1-yl)sulfonyl)phenoxy)ethyl)amino)methyl)-3-methyl-2-oxo-2,3-
dihydro-1H-benzo[d]imidazol-1-yl)piperidine-2,6-dione
3-(5-(4-(3-((4-((8-cyclopentyl-7-oxo-7,8-dihydropyrido[2,3-d]pyrimidin-2-
yl)amino)piperidin-1-yl)sulfonyl)benzyl)piperazin-1-yl)-6-fluoro-1-oxoisoindolin-2-
yl)piperidine-2,6-dione
5-(4-(3-((4-((8-cyclopentyl-7-oxo-7,8-dihydropyrido[2,3-d]pyrimidin-2-
yl)amino)piperidin-1-yl)sulfonyl)-5-fluorobenzyl)piperazin-1-yl)-2-(2,6-dioxopiperidin-3-
yl)-6-fluoroisoindoline-1,3-dione
2-(2,6-dioxopiperidin-3-yl)-5-fluoro-6-(4-(3-((4-((8-(2-hydroxy-2-methylcyclopentyl)-7-
oxo-7,8-dihydropyrido[2,3-d]pyrimidin-2-yl)amino)piperidin-1-
yl)sulfonyl)benzyl)piperazin-1-yl)isoindoline-1,3-dione
5-(4-(3-((4-((8-cyclopentyl-6-(difluoromethyl)-7-oxo-7,8-dihydropyrido[2,3-d]pyrimidin-
2-yl)amino)piperidin-1-yl)sulfonyl)benzyl)piperazin-1-yl)-2-(2,6-dioxopiperidin-3-yl)-6-
fluoroisoindoline-1,3-dione
8-cyclopentyl-2-((1-((3-((4-(2-(2,6-dioxopiperidin-3-yl)-6-fluoro-1,3-dioxoisoindolin-5-
yl)piperazin-1-yl)methyl)phenyl)sulfonyl)piperidin-4-yl)amino)-7-oxo-7,8-
dihydropyrido[2,3-d]pyrimidine-6-carbonitrile
5-(3-(3-((4-((8-cyclopentyl-7-oxo-7,8-dihydropyrido[2,3-d]pyrimidin-2-
yl)amino)piperidin-1-yl)sulfonyl)benzyl)-3,8-diazabicyclo[3.2.1]octan-8-yl)-2-(2,6-
dioxopiperidin-3-yl)-6-fluoroisoindoline-1,3-dione
5-(4-(3-((4-((8-cyclopentyl-7-oxo-7,8-dihydropyrido[2,3-d]pyrimidin-2-
yl)amino)piperidin-1-yl)sulfonyl)benzyl)piperazin-1-yl)-2-(2,6-dioxopiperidin-3-yl)-6-
fluoroisoindoline-1,3-dione
5-(8-(3-((4-((8-cyclopentyl-7-oxo-7,8-dihydropyrido[2,3-d]pyrimidin-2-
yl)amino)piperidin-1-yl)sulfonyl)benzyl)-3,8-diazabicyclo[3.2.1]octan-3-yl)-2-(2,6-
dioxopiperidin-3-yl)-6-fluoroisoindoline-1,3-dione
3-(5-(4-(3-((4-((8-cyclopentyl-7-oxo-7,8-dihydropyrido[2,3-d]pyrimidin-2-
yl)amino)piperidin-1-yl)sulfonyl)benzyl)piperazin-1-yl)-1-oxoisoindolin-2-yl)piperidine-
2,6-dione
3-(5-(5-(3-((4-((8-cyclopentyl-7-oxo-7,8-dihydropyrido[2,3-d]pyrimidin-2-
yl)amino)piperidin-1-yl)sulfonyl)benzyl)-2,5-diazabicyclo[2.2.2]octan-2-
yl)-1-oxoisoindolin-2-yl)piperidine-2,6-dione
3-(5-(3-(3-((4-((8-cyclopentyl-7-oxo-7,8-dihydropyrido[2,3-d]pyrimidin-2-
yl)amino)piperidin-1-yl)sulfonyl)benzyl)-3,8-diazabicyclo[3.2.1]octan-8-yl)-1-
oxoisoindolin-2-yl)piperidine-2,6-dione
3-(5-(5-(3-((4-((8-cyclopentyl-7-oxo-7,8-dihydropyrido[2,3-d]pyrimidin-2-
yl)amino)piperidin-1-yl)sulfonyl)benzyl)-2,5-diazabicyclo[2.2.1]heptan-2-yl)-1-
oxoisoindolin-2-yl)piperidine-2,6-dione
3-(5-(8-(3-((4-((8-cyclopentyl-7-oxo-7,8-dihydropyrido[2,3-d]pyrimidin-2-
yl)amino)piperidin-1-yl)sulfonyl)benzyl)-3,8-diazabicyclo[3.2.1]octan-3-yl)-1-
oxoisoindolin-2-yl)piperidine-2,6-dione
3-(5-(4-(3-((4-((8-(2-hydroxy-2-methylcyclopentyl)-7-oxo-7,8-dihydropyrido[2,3-
d]pyrimidin-2-yl)amino)piperidin-1-yl)sulfonyl)benzyl)piperazin-1-yl)-1-oxoisoindolin-2-
yl)piperidine-2,6-dione
3-(5-(4-(3-((4-((8-cyclopentyl-6-(difluoromethyl)-7-oxo-7,8-dihydropyrido[2,3-
d]pyrimidin-2-yl)amino)piperidin-1-yl)sulfonyl)benzyl)piperazin-1-yl)-1-oxoisoindolin-2-
yl)piperidine-2,6-dione
3-(5-(2-(3-((4-((8-cyclopentyl-7-oxo-7,8-dihydropyrido[2,3-d]pyrimidin-2-
yl)amino)piperidin-1-yl)sulfonyl)benzyl)-2,7-diazaspiro[3.5]nonan-7-yl)-1-oxoisoindolin-
2-yl)piperidine-2,6-dione
3-(5-(7-(3-((4-((8-cyclopentyl-7-oxo-7,8-dihydropyrido[2,3-d]pyrimidin-2-
yl)amino)piperidin-1-yl)sulfonyl)benzyl)-2,7-diazaspiro[3.5]nonan-2-yl)-1-oxoisoindolin-
2-yl)piperidine-2,6-dione
3-(5-(9-(3-((4-((8-cyclopentyl-7-oxo-7,8-dihydropyrido[2,3-d]pyrimidin-2-
yl)amino)piperidin-1-yl)sulfonyl)benzyl)-3,9-diazaspiro[5.5]undecan-3-yl)-1-
oxoisoindolin-2-yl)piperidine-2,6-dione
3-(5-(6-(3-((4-((8-cyclopentyl-7-oxo-7,8-dihydropyrido[2,3-d]pyrimidin-2-
yl)amino)piperidin-1-yl)sulfonyl)benzyl)-2,6-diazaspiro[3.3]heptan-2-yl)-1-oxoisoindolin-
2-yl)piperidine-2,6-dione
3-(5-(6-(3-((4-((8-(bicyclo[3.1.0]hexan-3-yl)-7-oxo-7,8-dihydropyrido[2,3-d]pyrimidin-2-
yl)amino)piperidin-1-yl)sulfonyl)benzyl)-2,6-diazaspiro[3.3]heptan-2-yl)-1-oxoisoindolin-
2-yl)piperidine-2,6-dione
3-(5-(6-(3-((4-((8-(bicyclo[1.1.1]pentan-1-yl)-7-oxo-7,8-dihydropyrido[2,3-d]pyrimidin-2-
yl)amino)piperidin-1-yl)sulfonyl)benzyl)-2,6-diazaspiro[3.3]heptan-2-yl)-1-oxoisoindolin-
2-yl)piperidine-2,6-dione
5-(4-(3-(((3R,4S)-4-((8-cyclopentyl-7-oxo-7,8-dihydropyrido[2,3-d]pyrimidin-2-
yl)amino)-3-fluoropiperidin-1-yl)sulfonyl)benzyl)piperazin-1-yl)-2-(2,6-dioxopiperidin-3-
yl)-6-fluoroisoindoline-1,3-dione
5-(4-(3-(((3S,4S)-4-((8-cyclopentyl-7-oxo-7,8-dihydropyrido[2,3-d]pyrimidin-2-
yl)amino)-3-methoxypiperidin-1-yl)sulfonyl)benzyl)piperazin-1-yl)-2-(2,6-dioxopiperidin-
3-yl)-6-fluoroisoindoline-1,3-dione
5-(4-(3-(((3S,4S)-4-((8-cyclopentyl-7-oxo-7,8-dihydropyrido[2,3-d]pyrimidin-2-
yl)amino)-3-fluoropiperidin-1-yl)sulfonyl)benzyl)piperazin-1-yl)-2-(2,6-dioxopiperidin-3-
yl)-6-fluoroisoindoline-1,3-dione
3-(5-((4-(3-((4-((8-cyclopentyl-7-oxo-7,8-dihydropyrido[2,3-d]pyrimidin-2-
yl)amino)piperidin-1-yl)sulfonyl)phenoxy)piperidin-1-yl)methyl)-1-oxoisoindolin-2-
yl)piperidine-2,6-dione
3-(5-((3-(3-((4-((8-cyclopentyl-7-oxo-7,8-dihydropyrido[2,3-d]pyrimidin-2-
yl)amino)piperidin-1-yl)sulfonyl)phenoxy)azetidin-1-yl)methyl)-1-oxoisoindolin-2-
yl)piperidine-2,6-dione
3-(5-(((2-(3-((4-((8-cyclopentyl-7-oxo-7,8-dihydropyrido[2,3-d]pyrimidin-2-
yl)amino)piperidin-1-yl)sulfonyl)phenoxy)ethyl)amino)methyl)-1-oxoisoindolin-2-
yl)piperidine-2,6-dione
3-(5-(3-(4-(3-((4-((8-cyclopentyl-7-oxo-7,8-dihydropyrido[2,3-d]pyrimidin-2-
yl)amino)piperidin-1-yl)sulfonyl)phenyl)piperazin-1-yl)azetidin-1-yl)-1-oxoisoindolin-2-
yl)piperidine-2,6-dione
3-(5-(4-(1-(3-((4-((8-cyclopentyl-7-oxo-7,8-dihydropyrido[2,3-d]pyrimidin-2-
yl)amino)piperidin-1-yl)sulfonyl)phenyl)azetidin-3-yl)piperazin-1-yl)-1-oxoisoindolin-2-
yl)piperidine-2,6-dione
1-(6-(1-(3-((4-((8-cyclopentyl-7-oxo-7,8-dihydropyrido[2,3-d]pyrimidin-2-
yl)amino)piperidin-1-yl)sulfonyl)benzyl)piperidin-4-yl)-1-methyl-1H-indazol-3-
yl)dihydropyrimidine-2,4(1H,3H)-dione
3-(4-(1-(1-(3-((4-((8-cyclopentyl-7-oxo-7,8-dihydropyrido[2,3-d]pyrimidin-2-
yl)amino)piperidin-1-yl)sulfonyl)phenyl)azetidin-3-yl)piperidin-4-yl)-3-methyl-2-oxo-2,3-
dihydro-1H-benzo[d]imidazol-1-yl)piperidine-2,6-dione
2-(2-(2-(2-((4-((8-cyclopentyl-7-oxo-7,8-dihydropyrido[2,3-d]pyrimidin-2-
yl)amino)piperidine)-1-sulfonamido)ethoxy)ethoxy)ethoxy)-N-(2-(2,6-dioxopiperidin-3-
yl)-1,3-dioxoisoindolin-4-yl)acetamide
3-(5-(3-(3-((4-((8-cyclopentyl-7-oxo-7,8-dihydropyrido[2,3-d]pyrimidin-2-
yl)amino)piperidin-1-yl)sulfonyl)phenoxy)azetidin-1-yl)-1-oxoisoindolin-2-yl)piperidine-
2,6-dione
3-(4-(1-((1-((4-((8-cyclopentyl-7-oxo-7,8-dihydropyrido[2,3-d]pyrimidin-2-
yl)amino)piperidin-1-yl)sulfonyl)piperidin-4-yl)methyl)piperidin-4-yl)-3-methyl-2-oxo-
2,3-dihydro-1H-benzo[d]imidazol-1-yl)piperidine-2,6-dione and
3-(5-(4-(3-(3-((4-((8-cyclopentyl-7-oxo-7,8-dihydropyrido[2,3-d]pyrimidin-2-
yl)amino)piperidin-1-yl)sulfonyl)phenoxy)azetidin-1-yl)piperidin-1-yl)-1-oxoisoindolin-2-
yl)piperidine-2,6-dione
or a pharmaceutically acceptable salt thereof.

42. A pharmaceutical composition comprising a compound of any one of claims 1 to 41, or a pharmaceutically acceptable salt thereof, and a pharmaceutically acceptable excipient.

43. A method of degrading CDK2 in a cell which method comprises contacting the cell with a compound of any one of claims 1 to 41, or a pharmaceutically acceptable salt thereof, or with a pharmaceutical composition of claim 42.

44. A method of treating cancer in a patient which method comprises administering to the patient in recognized need thereof, a therapeutically effective amount a compound of any one of claims 1 to 41, or a pharmaceutically acceptable salt thereof, or with a pharmaceutical composition of claim 42.

45. The method of claim 44, wherein a) the compound, or a pharmaceutically acceptable salt thereof, or b) the pharmaceutical composition is administered in combination with at least one other anticancer agent.

46. The method of claim 44 or 45, wherein the cancer is lung cancer, skin cancer, bladder cancer, breast cancer, cervical cancer, colorectal cancer, cancer of the small intestine, colon cancer, rectal cancer, cancer of the anus, endometrial cancer, gastric cancer, head and neck cancer, liver cancer, ovarian cancer, prostate cancer, testicular cancer, uterine cancer, esophageal cancer, gall bladder cancer, pancreatic cancer, stomach cancer, thyroid cancer, or parathyroid cancer.
